(19) United States
(12) Patent Application Publication
(10) Pub. No.: US 2022/0339169 A1
(43) Pub. Date: Oct. 27, 2022

(54) METHODS OF TREATING OR SELECTING A TREATMENT FOR A SUBJECT RESISTANT TO TNF INHIBITOR USING A NLRP3 ANTAGONIST

(71) Applicant: Novartis AG, Basel (CH)

(72) Inventors: Luigi FRANCHI, Ann Arbor, MI (US); Shomir GHOSH, Brookline, MA (US); Gary GLICK, Ann Arbor, MI (US); Jason KATZ, Newton, MA (US); Anthony William OPIPARI, Jr., Dexter, MI (US); William R. ROUSH, Boston, MA (US); Hans Martin SEIDEL, Concord, MA (US); Dong-Ming SHEN, Edison, NJ (US); Shankar VENKATRAMAN, Lansdale, PA (US); David Guenther WINKLER, Arlington, MA (US)

(21) Appl. No.: 17/252,630

(22) PCT Filed: Jul. 2, 2019
(86) PCT No.: PCT/US2019/040357

§ 371 Date: Dec. 15, 2020

Related U.S. Application Data
(60) Provisional application No. 62/693,886, filed on Jul. 3, 2018.
(60) Provisional application No. 62/693,879, filed on Jul. 3, 2018.
(60) Provisional application No. 62/702,602, filed on Jul. 24, 2018.
(60) Provisional application No. 62/702,621, filed on Jul. 24, 2018.

(51) Int. Cl. A61K 31/64; C07K 16/24; A61K 45/06; C12Q 1/6827; C12N 15/113

(52) U.S. Cl. CPC A61K 31/64; C07K 16/241; A61K 45/06; C12N 2310/12; C12N 15/113; C12N 2310/14; C12N 2310/11; C12Q 1/6827

(57) ABSTRACT

Provided herein are methods of treating a subject that include administering a therapeutically effective amount of an NLPR3 antagonist or a pharmaceutically acceptable salt, solvate, or co-crystal thereof to a subject identified as having a cell that has an elevated level of NLRP3 inflammasome activity and/or expression as compared to a reference level. Provided herein are methods of treating a subject, methods of selecting a treatment for a subject, methods of selecting a subject for treatment, and methods of selecting a subject for participation in a clinical study that include the administration of a therapeutically effective amount of an NLRP3 antagonist. Also provided are methods of treating a subject having resistance to an anti-TNFα agent and methods of determining the efficacy of treatment with an anti-TNFα agent. Also provided are methods of treating a subject with a combination of an NLRP3 antagonist and an anti-TNFα agent.

### TECHNICAL FIELD

The present disclosure relates to, in part, methods of treating a subject that include administration of an NLRP3 antagonist. The present disclosure also relates, in part, to methods, combinations and compositions for treating TFNα related diseases and anti-TNFα resistance in a subject that include administration of an NLRP3 antagonist, an NLRP3 antagonist and an anti-TNFα agent, or a composition comprising an NLRP3 antagonist and an anti-TNFα agent.

### BACKGROUND

The NLRP3 inflammasome is a component of the inflammatory process and its aberrant activation is pathogenic in inherited disorders such as the cryopyrin associated periodic syndromes (CAPS). The inherited CAPS Muckle-Wells syndrome (MWS), familial cold autoinflammatory syndrome (FCAS), and neonatal onset multi-system inflammatory disease (NOMID) are examples of indications that have been reported to be associated with gain-of-function mutations in NLRP3.

Alterations in NLRP3 have been associated with the pathogenesis of a number of complex diseases, including but not limited to intestinal diseases (e.g., Crohn's disease (CD), ulcerative colitis (UC), inflammatory bowel disease (IBD)), kidney disease (e.g., acute and chronic kidney injury), lung diseases (e.g., asthma, chronic obstructive pulmonary disease (COPD), pulmonary idiopathic fibrosis), liver diseases (e.g., nonalcoholic steatohepatitis (NASH), viral hepatitis, cirrhosis), metabolic disorders (e.g., type 2 diabetes, atherosclerosis, obesity, gout), musculoskeletal diseases (e.g., scleroderma), pancreatic diseases (e.g., acute and chronic pancreatitis), skin diseases (e.g., psoriasis), autoimmune diseases (e.g., rheumatoid arthritis, systemic lupus erythematosus (SLE), autoimmune thyroiditis, Addison's disease), diseases of the central nervous system, (e.g., Alzheimer's disease, multiple sclerosis, Amyotrophic Lateral Sclerosis and Parkinson disease), vessel disorders (e.g., giant cell arteritis), disorders of the bones (e.g., osteoarthritis, osteoporosis), eye diseases (e.g., glaucoma and macular degeneration), diseased caused by viral infection such as HIV and AIDS, pernicious anemia, cancer and aging.

Several patients having inflammatory or autoimmune diseases are treated with anti-TNFα agents. A subpopulation of such patients develop resistance to treatment with the anti-TNFα agents. It is desirable to develop methods for reducing a patient's resistance to anti-TNFα agents.

In light of the above, it would also be desirable to provide alternative therapies for treating inflammatory or autoimmune diseases (for example NLRP3 inflammasome inhibitors) to avoid or minimise the use of anti-TNFα agents.

Intestinal bowel disease (IBD), encompassing Ulcerative Colitis (UC) and Crohn's disease (CD), are chronic diseases characterized by barrier dysfunction and uncontrolled inflammation and mucosal immune reactions in the gut. A number of inflammatory pathways have been implicated in the progression of IBD, and anti-inflammatory therapy such as tumor necrosis factor-alpha (TNF-α) blockade has shown efficacy in the clinic (Rutgeerts P et al N Engl J Med 2005; 353:2462-76). Anti-TNFα therapies, however, do not show complete efficacy, however, other cytokines such as IL-1β, IL-6, IL-12, IL-18, IL-21, and IL-23 have been shown to drive inflammatory disease pathology in IBD (Neurath M F Nat Rev Immunol 2014; 14; 329-42). IL-1β and IL-18 are produced by the NLRP3 inflammasome in response to pathogenic danger signals, and have been shown to play a role in IBD. Anti-IL-1β therapy is efficacious in patients with IBD driven by genetic mutations in CARD8 or IL-10R (Mao L et al, J Clin Invest 2018; 238:1793-1806, Shouval D S et al, Gastroenterology 2016; 151:1100-1104), IL-18 genetic polymorphisms have been linked to UC (Kanai T et al, Curr Drug Targets 2013; 14:1392-9), and NLRP3 inflammasome inhibitors have been shown to be efficacious in murine models of IBD (Perera A P et al, Sci Rep 2018; 8:8618). Resident gut immune cells isolated from the lamina propria of IBD patients can produce IL-1β, either spontaneously or when stimulated by LPS, and this IL-1β production can be blocked by the ex vivo addition of a NLRP3 antagonist. Based on strong clinical and preclinical evidence showing that inflammasome-driven IL-1β and IL-18 play a role in IBD pathology, it is clear that NLRP3 inflammasome inhibitors could be an efficacious treatment option for UC, Crohn's disease, or subsets of IBD patients. These subsets of patients could be defined by their peripheral or gut levels of inflammasome related cytokines including IL-1β, IL-6, and IL-18, by genetic factors that pre-dispose IBD patients to having NLRP3 inflammasome activation such as mutations in genes including ATG16L1, CARDS, IL-10R, or PTPN2 (Saitoh T et al, Nature 2008; 456:264, Spalinger M R, Cell Rep 2018; 22:1835), or by other clinical rationale such as non-response to TNF therapy.

Though anti-TNF therapy is an effective treatment option for Crohn's disease, 40% of patients fail to respond. One-third of non-responsive CD patients fail to respond to anti-TNF therapy at the onset of treatment, while another third lose response to treatment over time (secondary non-response). Secondary non-response can be due to the generation of anti-drug antibodies, or a change in the immune compartment that desensitizes the patient to anti-TNF (Ben-Horin S et al, Autoimmun Rev 2014; 13:24-30, Steenholdt C et al Gut 2014; 63:919-27). Anti-TNF reduces inflammation in IBD by causing pathogenic T cell apoptosis in the intestine, therefore eliminating the T cell mediated inflammatory response (Van den Brande et al Gut 2007:56:509-17). There is increased NLRP3 expression and increased production of IL-10 in the gut of TNF-non-responsive CD patients (Leal R F et al Gut 2015; 64:233-42) compared to TNF-responsive patients, suggesting NLRP3 inflammasome pathway activation. Furthermore, there is increased expression of TNF-receptor 2 (TNF-R2), which allows for TNF-mediated proliferation of T cells (Schmitt H et al Gut 2018; 0:1-15). IL-10 signaling in the gut promotes T cell differentiation toward Th1/17 cells which can escape anti-TNF-α mediated apoptosis. It is therefore likely that NLRP3 inflammasome activation can cause non-responsiveness in CD patients to anti-TNF-α therapy by sensitizing pathogenic T cells in the gut to anti-TNF-α mediated apoptosis. Experimental data from immune cells isolated from the gut of TNF-resistant Crohn's patients show that these cells spontaneously release IL-1β, which can be inhibited by the addition of an NLRP3 antagonist. NLRP3 inflammasome antagonists—in part by blocking IL-1β secretion—would be expected to inhibit the mechanism leading to anti-TNF non-responsiveness, re-sensitizing the patient to anti-TNF therapy. In IBD patients who are naive to anti-TNF therapy, treatment with an NLRP3 antagonist would be expected to prevent primary- and secondary-non responsiveness by blocking the mechanism leading to non-response.

NLRP3 antagonists that are efficacious locally in the gut can be efficacious drugs to treat IBD; in particular in the treatment of TNF-resistant CD alone or in combination with anti-TNF therapy. Systemic inhibition of both IL-1β and TNF-α has been shown to increase the risk of opportunistic infections (Genovese M C et al, Arthritis Rheum 2004; 50:1412), therefore, only blocking the NLRP3 inflammasome at the site of inflammation would reduce the infection risk inherent in neutralizing both IL-1□ and TNF-α. NLRP3 antagonists that are potent in NLRP3-inflammasome driven cytokine secretion assays in cells, but have low permeability in vitro in a permeability assay such as an MDCK assay, have poor systemic bioavailability in a rat or mouse pharmacokinetic experiment, but high levels of compound in the colon and/or small intestine could be a useful therapeutic option for gut restricted purposes.

The present invention provides alternative therapies for the treatment of inflammatory or autoimmune diseases, including IBD, that solves the above problems associated with anti-TNFα agents.

### SUMMARY

The present invention is based on the discovery of mutations and mRNA/protein expression profiles that correlate with a subject's sensitivity to treatment with an NLRP3 antagonist.

The present invention is also relates to the Applicant's discovery that inhibition of NLRP3 inflammasomes can increase a subject's sensitivity to an anti-TNFα agent or can overcome resistance to an anti-TNFα agent in a subject, or indeed provide an alternative therapy to anti-TNFα agents.

Provided herein are methods of treating a subject that include: (a) identifying a subject having a cell that has an elevated level of NLRP3 inflammasome activity and/or expression as compared to a reference level; and (b) administering to the identified subject a therapeutically effective amount of an NLRP3 antagonist or a pharmaceutically acceptable salt, solvate, or co-crystal thereof.

Provided herein are methods for the treatment of inflammatory or autoimmune disease including IBD, such as UC and CD in a subject in need thereof, comprising administering to said subject a therapeutically effective amount of an NLRP3 agonist or a pharmaceutically acceptable salt, solvate, or co-crystal thereof, wherein the NLRP3 antagonist is a gut-targeted NLRP3 antagonist.

Provided herein are methods of treating a subject that include: administering a therapeutically effective amount of an NLPR3 antagonist or a pharmaceutically acceptable salt, solvate, or co-crystal thereof to a subject identified as having a cell that has an elevated level of NLRP3 inflammasome activity and/or expression as compared to a reference level.

Provided herein are methods of selecting a treatment for a subject that include: (a) identifying a subject having a cell that has an elevated level of NLRP3 inflammasome activity and/or expression as compared to a reference level; and (b) selecting for the identified subject a treatment comprising a therapeutically effective amount of an NLRP3 antagonist or a pharmaceutically acceptable salt, solvate, or co-crystal thereof.

Provided herein are methods of selecting a treatment for a subject, that include: selecting a treatment comprising a therapeutically effective amount of an NLRP3 antagonist or a pharmaceutically acceptable salt, solvate, or co-crystal thereof for a subject identified as having a cell that has an elevated level of NLRP3 inflammasome activity and/or expression as compared to a reference level.

Provided herein are methods of selecting a subject for treatment, that include: (a) identifying a subject having a cell that has an elevated level of NLRP3 inflammasome activity and/or expression as compared to a reference level; and (b) selecting an identified subject for treatment with a therapeutically effective amount of an NLRP3 antagonist or a pharmaceutically acceptable salt, solvate, or co-crystal thereof.

Provided herein are methods of selecting a subject for treatment, that include: selecting a subject identified as having a cell that has an elevated level of NLRP3 inflammasome activity and/or expression as compared to a reference level, for treatment with a therapeutically effective amount of an NLRP3 antagonist or a pharmaceutically acceptable salt, solvate, or co-crystal thereof.

Provided herein are methods of selecting a subject for participation in a clinical trial, that include: identifying a subject having a cancer cell that has an elevated level of NLRP3 inflammasome activity and/or expression as compared to a reference level; and selecting the identified subject for participation in a clinical trial that comprises administration of a therapeutically effective amount of a NLRP3 antagonist or a pharmaceutically acceptable salt, solvate, or co-crystal thereof.

Also provided herein are methods of selecting a subject for participation in a clinical trial, that include: selecting a subject identified as having a cell that has an elevated level of NLRP3 inflammasome activity and/or expression as compared to a reference level for participation in a clinical trial that comprises administration of a therapeutically effective amount of an NLRP3 antagonist or a pharmaceutically acceptable salt, solvate, or co-crystal thereof.

In some embodiments of any of the methods described herein, the subject identified as having a cell that has an elevated level of NLRP3 inflammasome activity has been determined to have a cell having a gain-of-function mutation in an NLRP3 gene.

In some embodiments of any of the methods described herein, the identifying a subject having a cell that has an elevated level of NLRP3 inflammasome activity includes detecting a loss-of-function mutation in a CARD8 gene in a cell from the subject.

In some embodiments of any of the methods described herein, the subject identified as having a cell that has an elevated level of NLRP3 inflammasome activity has been determined to have a cell having a loss-of-function mutation in a CARD8 gene.

In some embodiments of any of the methods described herein, the identifying a subject having a cell that has an elevated level of NLRP3 inflammasome activity includes detecting a gain-of-function mutation in an NLRP3 gene and a loss-of-function mutation in a CARD8 gene in a cell from the subject.

In some embodiments of any of the methods described herein, the subject identified as having a cell that has an elevated level of NLRP3 inflammasome activity has been determined to have a cell having a gain-of-function mutation in an NLRP3 gene and a loss-of-function mutation in a CARD8 gene.

In some embodiments of any of the methods described herein, the gain-of-function mutation in an NLRP3 gene results in the expression of a NLRP3 protein having a Q705K amino acid substitution.

In some embodiments of any of the methods described herein, the loss-of-function mutation in a CARD8 gene is a C allele at rs2043211.

In some embodiments of any of the methods described herein, the identifying a subject having a cell that has an elevated level of NLRP3 inflammasome expression includes detecting the level of one or more of NLRP3 protein, ASC protein, procaspase-1 protein, and caspase-1 protein.

In some embodiments of any of the methods described herein, identifying a subject having a cell that has an elevated level of NLRP3 inflammasome expression comprises detecting the level of one or more of NLRP3 mRNA, ASC mRNA, and procaspase-1 mRNA.

In some embodiments of any of the methods described herein, the subject identified as having a cell that has an elevated level of NLRP3 inflammasome expression has been determined to have a cell having an elevated level of one or more of NLRP3 protein, ASC protein, procaspase-1 protein, and caspase-1 protein.

In some embodiments of any of the methods described herein, the subject identified as having a cell that has an elevated level of NLRP3 inflammasome expression has been determined to have a cell having an elevated level of one or more of NLRP3 mRNA, ASC mRNA, and procaspase-1 mRNA.

In some embodiments of any of the methods described herein, the subject has or is suspected of having Crohn's disease, inflammatory bowel disease (IBD), or other gastrointestinal, autoimmune, or autoinflammatory disorders.

In some embodiments of any of the methods described herein, the identifying a subject having a cell that has an elevated level of NLRP3 inflammasome activity includes detecting a mutation in an NLRP3 gene that results in the expression of a NLRP3 protein having one or both of a T350M and a R262M amino acid substitution in a cell from the subject.

In some embodiments of any of the methods described herein, the subject identified as having a cell that has an elevated level of NLRP3 inflammasome activity has been determined to have a cell having an NLRP3 gene that results in the expression of a NLRP3 protein having one or both of a T350M and a R262M amino acid substitution.

In some embodiments of any of the methods described herein, the subject has or is suspected of having hereditary periodic fever.

In some embodiments of any of the methods described herein, the identifying a subject having a cell that has an elevated level of NLRP3 inflammasome activity includes detecting a mutation in an NLRP3 gene that results in the expression of an NLRP3 protein having one or more of a A441V, a V200M, a E629G, and a L355P amino acid substitution in a cell from the subject.

In some embodiments of any of the methods described herein, the subject identified as having a cell that has an elevated level of NLRP3 inflammasome activity has been determined to have a cell having an NLRP3 gene that results in the expression of an NLRP3 protein having one or more of a A441V, a V200M, a E629G, and a L355P amino acid substitution.

In some embodiments of any of the methods described herein, the subject has or is suspected of having familial cold autoinflammatory syndrome (FCAS).

In some embodiments of any of the methods described herein, the identifying a subject having a cell that has an elevated level of NLRP3 inflammasome activity comprises detecting a mutation in an NLRP3 gene that results in the expression of an NLRP3 protein having one or more of a R260W, a G571R, and a A354V amino acid substitution in a cell from the subject.

In some embodiments of any of the methods described herein, the subject identified as having a cell that has an elevated level of NLRP3 inflammasome activity has been determined to have a cell having an NLRP3 gene that results in the expression of an NLRP3 protein having one or more of a R260W, a G571R, and a A354V amino acid substitution.

In some embodiments of any of the methods described herein, the subject has or is suspected of having Muckle-Wells syndrome (MWS).

In some embodiments of any of the methods described herein, the identifying a subject having a cell that has an elevated level of NLRP3 inflammasome activity includes detecting a mutation in an NLRP3 gene that results in the expression of an NLRP3 protein having one or both of a D305N and a F311S amino acid substitution in a cell from the subject.

In some embodiments of any of the methods described herein, the subject identified as having a cell that has an elevated level of NLRP3 inflammasome activity has been determined to have a cell having an NLRP3 gene that results in the expression of an NLRP3 protein having one or both of a D305N and a F311S amino acid substitution.

In some embodiments of any of the methods described herein, the subject has or is suspected of having Cinca syndrome.

In some embodiments of any of the methods described herein, the identifying a subject having a cell that has an elevated level of NLRP3 inflammasome activity includes detecting a mutation in an NLRP3 gene that results in the expression of an NLRP3 protein having one or both of a R920Q amino acid substitution in a cell from the subject.

In some embodiments of any of the methods described herein, the subject identified as having a cell that has an elevated level of NLRP3 inflammasome activity has been determined to have a cell having a mutation in an NLRP3 gene that results in the expression of an NLRP3 protein having a R920Q amino acid substitution.

In some embodiments of any of the methods described herein, the subject has or is suspected of having deafness with or without inflammation.

In some embodiments of any of the methods described herein, the identifying a subject having a cell that has an elevated level of NLRP3 inflammasome activity includes detecting a mutation in an NLRP3 gene that results in the expression of an NLRP3 protein having a D21H amino acid substitution in a cell from the subject.

In some embodiments of any of the methods described herein, the subject identified as having a cell that has an elevated level of NLRP3 inflammasome activity has been determined to have a cell having an NLRP3 gene that results in the expression of an NLRP3 protein having a D21H amino acid substitution.

In some embodiments of any of the methods described herein, the subject has or is suspected of having keratoendotheliitis fugax hereditaria.

In some embodiments of any of the methods described herein, the subject has or is suspected of having an inappropriate host response to infectious diseases where active infection exists at any body site. In some embodiments, the inappropriate host response to infectious disease where active infection exists at any body site is selected from the group consisting of: septic shock, disseminated intravascular coagulation, and adult respiratory distress syndrome.

In some embodiments of any of the methods described herein, the subject has or is suspected of having acute or chronic inflammation due to antigen, antibody, and/or complement deposition.

In some embodiments of any of the methods described herein, the subject has or is suspected of having an inflammatory disease or condition. In some embodiments, the inflammatory disease or condition is selected from the group consisting of: arthritis, cholangitis, colitis, encephalitis, endocarditis, glomerulonephritis, hepatitis, myocarditis, pancreatitis, pericarditis, reperfusion injury, vasculitis, osteoarthritis, COPD, periodontal disease, uveitis, cutaneous T-cell lymphoma, and mucositis such as oral mucositis, esophageal mucositis, and intestinal mucositis.

In some embodiments of any of the methods described herein, the subject has or is suspected of having acute and delayed hypersensitivity, graft rejection, or graft-versus-host disease (GVHD).

In some embodiments of any of the methods described herein, the subject has or is suspected of having an autoimmune disease selected from the group consisting of: Type 1 diabetes mellitus, rheumatoid arthritis, systemic lupus erythematosus, autoimmune thyroiditis, Addison's disease, pernicious anemia, multiple sclerosis, an inflammatory bowel disease (IBD), scleroderma, and psoriasis. In some embodiments, the IBD is selected from the group consisting of: Crohn's disease, ulcerative colitis, autoimmune colitis, iatrogenic autoimmune colitis, ulcerative colitis, colitis induced by one or more chemotherapeutic agents, colitis induced by treatment with adoptive cell therapy, colitis associated with one or more alloimmune diseases such as GVHD, radiation enteritis, collagenous colitis, lymphocytic colitis, microscopic colitis, and radiation enteritis, celiac disease, and inflammatory bowel syndrome.

In some embodiments of any of the methods described herein, the subject has or is suspected of having a metabolic disorder. In some embodiments, the metabolic disorder selected from the group consisting of type 2 diabetes, atherosclerosis, obesity, gout, and pseudogout.

In some embodiments of any of the methods described herein, the subject has or is suspected of having a disease of the central nervous system.

In some embodiments, the disease of the central nervous system is selected from the group consisting of: Alzheimer's disease, multiple sclerosis, amyotrophic lateral sclerosis, and Parkinson's disease.

In some embodiments of any of the methods described herein, the subject has or is suspected of having a lung disease. In some embodiments, the lung disease is asthma, COPD, pulmonary idiopathic fibrosis, or cystic fibrosis.

In some embodiments of any of the methods described herein, the subject has or is suspected of having a liver disease.

In some embodiments, the liver disease is selected from the group consisting of: NASH syndrome, viral hepatitis, and cirrhosis.

In some embodiments of any of the methods described herein, the subject has or is suspected of having a pancreatic disease, such as acute or chronic pancreatitis.

In some embodiments of any of the methods described herein, the subject has or is suspected of having kidney disease, such as acute or chronic kidney injury.

In some embodiments of any of the methods described herein, the subject has or is suspected of having an intestinal disease, such as Crohn's disease or ulcerative colitis.

In some embodiments of any of the methods described herein, the subject has or is suspected of having a skin disease, such as psoriasis.

In some embodiments of any of the methods described herein, the subject has or is suspected of having a musculoskeletal disease, such as scleroderma.

In some embodiments of any of the methods described herein, the subject has or is suspected of having a vessel disorder, such as giant cell arteritis.

In some embodiments of any of the methods described herein, the subject has a bone disorder, such as osteoarthritis, osteoporosis, and osteopetrosis disorders.

In some embodiments of any of the methods described herein, the subject has or is suspected of having an eye disease, such as glaucoma or macular degeneration, such as age-related macular degeneration.

In some embodiments of any of the methods described herein, the subject has or is suspected of having a disease caused by viral infection, such as HIV or AIDS.

In some embodiments of any of the methods described herein, the subject has or is suspected of having a cancer, such as non-small cell lung cancer, acute lymphoblastic leukemia (ALL) (ALL in patients resistant to glucocorticoid treatment), multiple myeloma, promyelocytic leukemia, gastric cancer, and lung cancer metastasis.

In some embodiments of any of the methods described herein, the subject has or is suspected of having a cardiovascular disease.

In some embodiments, the cardiovascular disease is myocardial infarction, stroke, or heart failure.

In some embodiments of any of the methods described herein, the subject has or is suspected of having: hereditary periodic fever, familial cold autoinflammatory syndrome (FCAS), Muckle-Wells syndrome, myelodysplastic syndrome (MDS), Langerhan's cell histiocytosis (LCH), neonatal onset multisystem inflammatory disease, Cinca syndrome, deafness with inflammation, deafness without inflammation, keratoendotheliitis fugax hereditaria, silicosis, asbestosis, or chronic neurologic cutaneous and articular syndrome.

In some embodiments of any of the methods described herein, the subject has been exposed to, or is suspected of having been exposed to, a toxic agent selected from the group consisting of: exogenous microbial stimuli, lipopolysaccharide (LPS), lipooligosaccharide, muramyl dipeptide (MDP), nigericin, maitotoxin, asbestos, and silica.

Provided herein are methods of treating a subject in need thereof, that include: (a) identifying a subject having resistance to an anti-TNFα agent; and (b) administering a treatment comprising a therapeutically effective amount of an NLRP3 antagonist or a pharmaceutically acceptable salt, solvate, or co-crystal thereof to the identified subject.

Provided herein are methods of treating a subject in need thereof, that include: administering a treatment comprising a therapeutically effective amount of an NLRP3 antagonist or a pharmaceutically acceptable salt, solvate, or co-crystal thereof to a subject identified as having resistance to an anti-TNFα agent.

Provided herein are methods of selecting a treatment for a subject in need thereof, that include: (a) identifying a subject having resistance to an anti-TNFα agent; and (b) selecting for the identified subject a treatment comprising a therapeutically effective amount of an NLRP3 antagonist or a pharmaceutically acceptable salt, solvate, or co-crystal thereof.

Provided herein are methods of selecting a treatment for a subject in need thereof, that include selecting a treatment comprising a therapeutically effective amount of an NLRP3 antagonist or a pharmaceutically acceptable salt, solvate, or co-crystal thereof for a subject identified as having resistance to an anti-TNFα agent.

Provided herein are methods of selecting a subject for treatment, that include: (a) identifying a subject having resistance to an anti-TNFα agent; and (b) selecting the identified subject for treatment with a therapeutically effective amount of an NLRP3 antagonist or a pharmaceutically acceptable salt, solvate, or co-crystal thereof.

Provided herein are methods of selecting a subject for treatment, that include selecting a subject identified as having resistance to an anti-TNFα agent, for treatment with a therapeutically effective level of an NLRP3 antagonist or a pharmaceutically acceptable salt, solvate, or co-crystal thereof.

Provided herein are methods of selecting a subject for participation in a clinical trial, that include: (a) identifying a subject having resistance to an anti-TNFα agent; and (b) selecting the identified subject for participation in a clinical trial that includes administration of a treatment comprising a therapeutically effective amount of an NLRP3 antagonist or a pharmaceutically acceptable salt, solvate, or co-crystal thereof.

Provided herein are methods of selecting a subject for participation in a clinical trial, that include selecting a subject identified as having resistance to an anti-TNFα agent, for participation in a clinical trial that includes administration of a treatment comprising a therapeutically effective amount of an NLRP3 antagonist or a pharmaceutically acceptable salt, solvate, or co-crystal thereof.

In some embodiments of any of the methods described herein, step (b) can further include identifying the subject as also having an elevated level of NLRP3 inflammasome activity and/or expression in a cell obtained from the subject, as compared to a reference level.

In some embodiments of any of the methods described herein, the identified subject also has an elevated level of NLRP3 inflammasome activity and/or expression in a cell obtained from the subject, as compared to a reference level.

In some embodiments, the identifying the subject as also having a cell that has an elevated level of NLRP3 inflammasome expression includes detecting the level of one or more of NLRP3 protein, ASC protein, procaspase-1 protein, and caspase-1 protein.

In some embodiments, the identifying the subject as also having a cell that has an elevated level of NLRP3 inflammasome expression includes detecting the level of one or more of NLRP3 mRNA, ASC mRNA, and procaspase-1 mRNA.

In some embodiments, the identified subject also having a cell that has an elevated level of NLRP3 inflammasome expression has been determined to have a cell having an elevated level of one or more of NLRP3 protein, ASC protein, procaspase-1 protein, and capsase-1 protein.

In some embodiments, the identified subject also having a cell that has an elevated level of NLRP3 inflammasome expression has been determined to have a cell having an elevated level of one or more of NLRP3 mRNA, ASC mRNA, and procaspase-1 mRNA.

In some embodiments of any of the methods described herein, the treatment further includes a therapeutically effective amount of an anti-TNFα agent, in addition to the NLRP3 antagonist.

Provided herein are methods of treating a subject in need thereof, that include: (a) administering one or more doses of an anti-TNFα agent to the subject; (b) detecting an elevated level of NLRP3 inflammasome activity and/or expression in a cell obtained from the subject after step (a) as compared to a reference level; and (c) administering a treatment comprising a therapeutically effective amount of an NLRP3 antagonist or a pharmaceutically acceptable salt, solvate, or co-crystal thereof to a subject determined to have an elevated level of NLRP3 inflammasome activity and/or expression as compared to the reference level in step (b).

Provided herein are methods of treating a subject in need thereof, that include: (a) detecting an elevated level of NLRP3 inflammasome activity and/or expression as compared to a reference level in a cell obtained from a subject previously administered one or more doses of an anti-TNFα agent; and (b) administering a treatment comprising a therapeutically effective amount of an NLRP3 antagonist or a pharmaceutically acceptable salt, solvate, or co-crystal thereof to a subject determined to have an elevated level of NLRP3 inflammasome activity and/or expression as compared to the reference level in step (a).

Provided herein are methods of treating a subject in need thereof, that include administering a treatment comprising a therapeutically effective amount of an NLRP3 antagonist or a pharmaceutically acceptable salt, solvate, or co-crystal thereof to a subject determined to have an elevated level of NLRP3 inflammasome activity and/or expression as compared to a reference level in a cell obtained from the subject after previous administration with one or more doses of an anti-TNFα agent.

In some embodiments of any of the methods described herein, the detecting an elevated level of NLRP3 inflammasome expression as compared to the reference level expression includes detecting the level of one or more of NLRP3 protein, ASC protein, procaspase-1 protein, and caspase-1 protein.

In some embodiments of any of the methods described herein, the identifying the subject as also having a cell that has an elevated level of NLRP3 inflammasome expression includes detecting the level of one or more of NLRP3 mRNA, ASC mRNA, and procaspase-1 mRNA.

In some embodiments of any of the methods described herein, the subject determined to have an elevated level of NLRP3 inflammasome expression as compared to a reference level has been determined to have a cell having an elevated level of one or more of NLRP3 protein, ASC protein, procaspase-1 protein, and caspase-1 protein.

In some embodiments, the subject determined to have an elevated level of NLRP3 inflammasome expression as compared to a reference level has been determined to have a cell having an elevated level of one or more of NLRP3 mRNA, ASC mRNA, and procaspase-1 mRNA.

In some embodiments of any of the methods described herein, the treatment further includes a therapeutically effective amount of an anti-TNFα agent, in addition to the NLRP3 antagonist.

Provided herein are methods of treating a subject in need thereof, that include: (a) administering one or more doses of an anti-TNFα agent to the subject; (b) after step (a), detecting resistance to the anti-TNFα agent in the subject; and (c) administering a treatment comprising a therapeutically effective amount of an NLRP3 antagonist or a pharmaceutically acceptable salt, solvate, or co-crystal thereof to a subject determined to have resistance to the anti-TNFα agent in step (b).

Provided herein are methods of treating a subject in need thereof, that include: (a) detecting resistance to an anti-TNFα agent in a subject previously administered one or more doses of the anti-TNFα agent; and (b) administering a treatment comprising a therapeutically effective amount of an NLRP3 antagonist or a pharmaceutically acceptable salt, solvate, or co-crystal thereof to a subject determined to have resistance to the anti-TNFα agent in step (a).

Provided herein are methods of treating a subject in need thereof, that include: administering a treatment comprising a therapeutically effective amount of an NLRP3 antagonist or a pharmaceutically acceptable salt, solvate, or co-crystal thereof to a subject previously administered one or more doses of an anti-TNFα agent and determined to have resistance to the anti-TNFα agent.

In some embodiments of any of the methods described herein, the treatment further includes a therapeutically effective amount of an anti-TNFα agent, in addition to the NLRP3 antagonist.

Provided herein are methods of reducing the risk of developing resistance to an anti-TNFα agent in a subject in need thereof, that include: administering to a subject in need thereof a therapeutically effective amount of an anti-TNFα agent and a therapeutically effective amount of an NLRP3 antagonist or a pharmaceutically acceptable salt, solvate, or co-crystal thereof.

In some embodiments, the anti-TNFα agent and the NLRP3 antagonist or a pharmaceutically acceptable salt, solvate, or co-crystal thereof are administered at substantially the same time.

In some embodiments, the anti-TNFα agent and the NLRP3 antagonist or a pharmaceutically acceptable salt, solvate, or co-crystal thereof are formulated into a single dosage form.

In some embodiments, the NLRP3 antagonist or a pharmaceutically acceptable salt, solvate, or co-crystal thereof is administered to the subject prior to administration of the anti-TNFα agent.

In some embodiments, the anti-TNFα agent is administered to the subject prior to administration of the NLRP3 antagonist or a pharmaceutically acceptable salt, solvate, or co-crystal thereof.

Provided herein are methods of predicting a subject's responsiveness to an anti-TNFα agent, that include: (a) determining that a subject has an elevated level of NLRP3 inflammasome activity and/or expression in a cell obtained from the subject, as compared to a reference level; and (b) identifying that the subject determined to have an elevated level of NLRP3 inflammasome activity and/or expression in step (a) has an increased likelihood of being resistant to treatment with an anti-TNFα agent.

Provided herein are methods of predicting a subject's responsiveness to an anti-TNFα agent, that include identifying a subject determined to have an elevated level of NLRP3 inflammasome activity and/or expression in a cell obtained from the subject, as having an increased likelihood of being resistant to treatment with an anti-TNFα agent.

In some embodiments, the determining that a subject has an elevated level of NLRP3 inflammasome activity and/or expression includes detecting the level of one or more of NLRP3 protein, ASC protein, procaspase-1 protein, and caspase-1 protein.

In some embodiments, the determining that a subject has an elevated level of NLRP3 inflammasome expression includes detecting the level of one or more of NLRP3 mRNA, ASC mRNA, and procaspase-1 mRNA.

In some embodiments, the subject determined to have an elevated level of NLRP3 inflammasome expression has been determined to have a cell having an elevated level of one or more of NLRP3 protein, ASC protein, procaspase-1 protein, and capsase-1 protein.

In some embodiments, the subject determined to have an elevated level of NLRP3 inflammasome expression has been determined to have a cell having an elevated level of one or more of NLRP3 mRNA, ASC mRNA, and procaspase-1 mRNA.

In some embodiments of any of the methods described herein, the subject has not previously been administered a dose of an anti-TNFα antagonist.

In some embodiments of any of the methods described herein, the method further includes administering to the subject identified as having an increased likelihood of being resistant to treatment with an anti-TNFα agent, a treatment comprising (i) a therapeutically effective amount of an anti-TNFα agent and (ii) a therapeutically effective amount of an NLRP3 antagonist.

Provided herein are methods of treating a subject in need thereof, that include: (a) identifying a subject having an elevated level of NLRP3 inflammasome activity and/or expression in a cell obtained from the subject, as compared to a reference level; and (b) administering a treatment comprising (i) a therapeutically effective amount of an NLRP3 antagonist or a pharmaceutically acceptable salt, solvate, or co-crystal thereof, and (ii) a therapeutically effective amount of an anti-TNFα agent to the identified subject.

Provided herein are methods of treating a subject in need thereof, that include: administering a treatment comprising (i) a therapeutically effective amount of an NLRP3 antagonist or a pharmaceutically acceptable salt, solvate, or co-crystal thereof, and (ii) a therapeutically effective amount of an anti-TNFα agent, to a subject identified as having an elevated level of NLRP3 inflammasome activity and/or expression in a cell obtained from the subject, as compared to a reference level.

Provided herein are methods of selecting a treatment for a subject in need thereof, that include: (a) identifying a subject having an elevated level of NLRP3 inflammasome activity and/or expression in a cell obtained from the subject, as compared to a reference level; and (b) selecting for the identified subject a treatment comprising (i) a therapeutically effective amount of an NLRP3 antagonist or a pharmaceutically acceptable salt, solvate, or co-crystal thereof, and (ii) a therapeutically effective amount of an anti-TNFα agent.

Provided herein are methods of selecting a treatment for a subject in need thereof, that include: selecting a treatment comprising (i) a therapeutically effective amount of an NLRP3 antagonist or a pharmaceutically acceptable salt, solvate, or co-crystal thereof, and (ii) a therapeutically effective amount of an anti-TNFα agent, for a subject identified as having an elevated level of NLRP3 inflammasome activity and/or expression in a cell obtained from the subject, as compared to a reference level.

Provided herein are methods of selecting a subject for treatment, that include: (a) identifying a subject having an elevated level of NLRP3 inflammasome activity and/or expression in a cell obtained from the subject, as compared to a reference level; and (b) selecting the identified subject for treatment with (i) a therapeutically effective amount of an NLRP3 antagonist or a pharmaceutically acceptable salt, solvate, or co-crystal thereof, and (ii) a therapeutically effective amount of an anti-TNFα agent.

Provided herein are methods of selecting a subject for treatment, that include: selecting a subject identified as having an elevated level of NLRP3 inflammasome activity and/or expression in a cell obtained from the subject, as compared to a reference level, for treatment with (i) a therapeutically effective level of an NLRP3 antagonist or a pharmaceutically acceptable salt, solvate, or co-crystal thereof, and (ii) a therapeutically effective amount of an anti-TNFα agent.

Also provided herein are methods of selecting a subject for participation in a clinical trial, that include: (a) identifying a subject having an elevated level of NLRP3 inflammasome activity and/or expression in a cell obtained from the subject, as compared to a reference level; and (b) selecting the identified subject for participation in a clinical trial that includes administration of a treatment comprising (i) a therapeutically effective amount of an NLRP3 antagonist or a pharmaceutically acceptable salt, solvate, or co-crystal thereof, and (ii) a therapeutically effective amount of an anti-TNFα agent.

Also provided herein are methods of selecting a subject for participation in a clinical trial, that include: selecting a subject identified as having an elevated level of NLRP3 inflammasome activity and/or expression in a cell obtained from the subject, as compared to a reference level, for participation in a clinical trial that includes administration of a treatment comprising (i) a therapeutically effective amount of an NLRP3 antagonist or a pharmaceutically acceptable salt, solvate, or co-crystal thereof, and (ii) a therapeutically effective amount of an anti-TNFα agent.

In some embodiments of any of the methods described herein, the NLRP3 inflammasome activity is secretion of IL-18.

In some embodiments of any of the methods described herein, the NLRP3 inflammasome activity is secretion of IL-1β.

In some embodiments of any of the methods described herein, the NLRP3 inflammasome activity is caspase-1 activity.

In some embodiments of any of the methods described herein, the NLRP3 inflammasome activity is the level of lipocalin-2.

In some embodiments of any of the methods described herein, the NLRP3 inflammasome activity is the level of S100A8.

In some embodiments of any of the methods described herein, the NLRP3 inflammasome activity is the level of S100A9.

In some embodiments of any of the methods described herein, the identifying the subject as having a cell that has an elevated level of NLRP3 inflammasome expression includes detecting the level of one or more of NLRP3 protein, ASC protein, procaspase-1 protein, and caspase-1 protein.

In some embodiments of any of the methods described herein, the identifying the subject as having a cell that has an elevated level of NLRP3 inflammasome expression includes detecting the level of one or more of NLRP3 mRNA, ASC mRNA, and procaspase-1 mRNA.

In some embodiments of any of the methods described herein, the identified subject having a cell that has an elevated level of NLRP3 inflammasome expression has been determined to have a cell having an elevated level of one or more of NLRP3 protein, ASC protein, procaspase-1 protein, and capsase-1 protein.

In some embodiments of any of the methods described herein, the identified subject having a cell that has an elevated level of NLRP3 inflammasome expression has been determined to have a cell having an elevated level of one or more of NLRP3 mRNA, ASC mRNA, and procaspase-1 mRNA.

In some embodiments of any of the methods described herein, the subject has not previously been administered an anti-TNFα agent.

In some embodiments of any of the methods described herein, the subject has been diagnosed or identified as having an inflammatory or an autoimmune disorder.

In some embodiments, the inflammatory or autoimmune disorder is selected from the group consisting of: sickle cell disease, rheumatoid arthritis, juvenile arthritis, psoriatic arthritis, plaque psoriasis, ankylosing spondylitis, ulcerative colitis, Crohn's disease, inflammatory bowel disease, Behcet's disease, Takayasu's arteritis, atherosclerosis, gout, psoriasis, an infectious disease, asthma, peptic ulcer, periodontitis, dermatitis, diverticulitis, fibromyalgia, hepatitis, systemic lupus erythematosus, nephritis, appendicitis, bursitis, cystitis, encephalitis, gingivitis, meningitis, myelitis, neuritis, pharyngitis, phlebitis, prostatitis, rhinitis, sinusitis, tendonitis, testiculitis, tonsillitis, urethritis, vasculitis, vaginitis, Celiac disease, diverticulitis, glomerulonephritis, hidradenitis suppurativa, hypersensitivities, interstitial cystitis, Lichen planus, mast cell activation syndrome, mastocystosis, otitis, pelvic inflammatory disease, reperfusion injury, rheumatic fever, rhinitis, sarcoidosis, graft versus host disease, vasculitis, allergy, cancer, HIV, AIDS, scleroderma, Sjøgren's syndrome, anti-phospholipid antibody syndrome, myocarditis, postmyocardial infarction syndrome, postpericardiotomy syndrome, subacute bacterial endocarditis, anti-glomerular basement membrane nephritis, interstitial cystitis, lupus nephritis, autoimmune nephritis, autoimmune hepatitis, autoimmune hepatitis, primary biliary cholangitis, primary sclerosing cholangitis, primary schlerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, Lichen sclerosus, linear IgA disease, morphea, pemphigus vulgaris, pityriasis lichenoides et varioliforms acuta, Mucha-Habermann disease, psoriasis, systemic scleroderma, vitiligo, Addison's disease, autoimmune polyendocrine syndrome type 1, 2, or 3, autoimmune pancreatitis, diabetes mellitus type 1, autoimmune thyroiditis, Ord's thyroiditis, Graves' disease, autoimmune oophoritis, endometriosis, autoimmune orchitis, Sjogren's syndrome, autoimmune enteropathy, microscopic colitis, antiphospholipid syndrome, aplastic anemia, autoimmune hemolytic anemia, autoimmune lymphoproliferative syndrome, autoimmune neutropenia, autoimmune thrombocytopenic purpura, cold agglutinin disease, essential mixed cyroglobulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, Lyme disease, mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, sarcoidosis, Schnitzler syndrome, undifferentiated connective tissue disease, dermatomyositis, fibromyalgia, inclusion body myositis, myasthenia gravis, neuromyotonia, paraneoplastic cerebellar degeneration, polymyositis, acute disseminated encephalomyelitis, acute motor axonal neuropathy, anti-N-methyl-D-aspartate receptor encephalitis, Balo concentric sclerosis, Bickerstaff's encephalitis, chronic inflamatory demyelinating polyneuropathy, Guillain-Barre syndrome, Hashimoto's encephalopathy, idiopathic inflammatory demyelinating disease, Lambert-Eaton myasthenic syndrome, multiple sclerosis, Oshtoran syndrome, pediatric autoimmune neuropsychiatric disorder associated with Streptococcus, progressive inflammatory neuropathy, restless leg syndrome, Stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, Opsoclonus myoclonus syndrome, optic neuritis, scleritis, Susac's syndrome, sympathetic ophthalmia, Tolosa-Hunt syndrome, autoimmune inner ear disease, Meniere's disease, Behcet's disease, eosinophilic graunulomatosis with polyangiitis, giant cell arteritis, graunulomatosis with polyangiitis, IgA vasculitis, Kawasaki's disease, leukocytoclastic vasculitis, lupus vasculitis, microscopic polyangiitis, polyarteritis nodosa, polymyalgia rheumatica, urticarial vasculitis, vasculitis, primary immune deficiency, chronic fatigue syndrome, complex regional pain syndrome, eosinophilic esophagitis, gastritis, interstitial lung disease, POEMS syndrome, Raynaud's phenomenon, primary immunodeficiency, and pyoderma gangrenosum.

In some embodiments, the inflammatory or autoimmune disorder is Crohn's disease or ulcerative colitis.

In some embodiments, the inflammatory or autoimmune disorder is inflammatory bowel syndrome.

In some embodiments of any of the methods described herein, the anti-TNFα agent is an antibody or an antigen-binding antibody fragment, or a soluble TNFα receptor.

In some embodiments, the antibody is selected from the group consisting of: adalimumab, certolizumab, etanercept, golimumab, infliximab, CDP571, and certolizumab pegol.

In some embodiments of any of the methods described herein, the anti-TNFα agent is a small molecule inhibitor of a signaling component downstream of a TNFα receptor.

In some embodiments of any of the methods described herein, the NLRP3 antagonist is an inhibitory nucleic acid. In some embodiments, the inhibitory nucleic acid is a short interfering RNA, an antisense nucleic acid, or a ribozyme.

In some embodiments of any of the methods described herein, the NLRP3 antagonist is a compound of any one of Formulas I-XII, or a pharmaceutically acceptable salt, solvate, or co-crystal thereof.

In some embodiments of any of the methods described herein, the NLRP3 antagonist is a compound shown in any one of Tables 1-18, or a pharmaceutically acceptable salt, solvate, or co-crystal thereof.

In some embodiments of any of the methods described herein, the NLRP3 antagonist is a compound referred to or shown herein, or a pharmaceutically acceptable salt, solvate, or co-crystal thereof. As used herein, the term “antagonist of NLRP3” is an agent, a genetic mutation, or altered signaling pathways in a mammalian cell that results in a decrease in one or both of (i) the activity of an NLRP3 inflammasome (e.g., any of the exemplary activities of an NLRP3 inflammasome described herein) (e.g., as compared to the level of NLRP3 inflammasome activity in the absence of the agent) and (ii) the expression level of NLRP3 inflammasomes in a mammalian cell (e.g., using any of the exemplary methods of detection described herein) (e.g., as compared to the expression level of NLRP3 inflammasomes in a mammalian cell not contacted with the agent). Non-limiting examples of NLRP3 antagonists are described herein.

As used herein, the term “NLRP3” is meant to include, without limitation, nucleic acids, polynucleotides, oligonucleotides, sense and antisense polynucleotide strands, complementary sequences, peptides, polypeptides, proteins, homologous and/or orthologous NLRP molecules, isoforms, precursors, mutants, variants, derivatives, splice variants, alleles, different species, and active fragments thereof.

The term “NLRP3 inflammasome expression” means the level of one or more of NLRP3 protein, ASC protein, procaspase-1 protein, caspase-1 protein, NLRP3 mRNA, ASC mRNA, and procaspase-1 mRNA in a mammalian cell (e.g., a mammalian cell obtained from a subject).

The term “acceptable” with respect to a formulation, composition, or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of an NLRP3 antagonist being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising an NLRP3 antagonist disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “pharmaceutically acceptable salt” may refer to pharmaceutically acceptable addition salts prepared from pharmaceutically acceptable non-toxic acids including inorganic and organic acids. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. The term “pharmaceutically acceptable salt” may also refer to pharmaceutically acceptable addition salts prepared by reacting a compound having an acidic group with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salts not specifically limited as far as it can be used in medicaments. Examples of a salt that the compounds described herein from with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of an NLRP3 antagonistor other compound described herein with other chemical components (referred to collectively herein as “excipients”), such as carriers, stabilizers, diluents, dispersing agents, suspending agents, and/or thickening agents. The pharmaceutical composition facilitates administration of the NLRP3 antagonist or other compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human. In some embodiments of any of the methods described herein, the subject is 1 year old or older, 2 years old or older, 4 years old or older, 5 years old or older, 10 years old or older, 12 years old or older, 13 years old or older, 15 years old or older, 16 years old or older, 18 years old or older, 20 years old or older, 25 years old or older, 30 years old or older, 35 years old or older, 40 years old or older, 45 years old or older, 50 years old or older, 55 years old or older, 60 years old or older, 65 years old or older, 70 years old or older, 75 years old or older, 80 years old or older, 85 years old or older, 90 years old or older, 95 years old or older, 100 years old or older, or 105 years old or older,

In some embodiments of any of the methods described herein, the subject has been previously diagnosed or identified as having a disease associated with NLRP3 inflammasome activity (e.g., any of the types of NLRP3 inflammasome activity associated-diseases described herein or known in the art, e.g., an inflammatory disease or an autoimmune disease). In some embodiments of any of the methods described herein, the subject is suspected of having a NLRP3 inflammasome activity-associated disease (e.g., any of the types of NLRP3 inflammasome activity-associated diseases described herein or known in the art, e.g., an inflammatory disease or an autoimmune disease). In some embodiments of any of the methods described herein, the subject is presenting with one or more (e.g., two, three, four, or five) symptoms of a NLRP3 inflammasome activity-associated disease (e.g., any of the NLRP3 inflammasome activity-associated disease described herein or known in the art).

In some embodiments of any of the methods described herein, the subject has been previously diagnosed or identified as having a disease associated with an elevated level of TNFα activity and/or expression (e.g., any of the types of TNFα associated-diseases described herein or known in the art). In some embodiments of any of the methods described herein, the subject has been previously diagnosed or identified as having a disease associated with resistance to an anti-TNFα agent (e.g., any of the anti-TNFα agent described herein or known in the art).

In some embodiments of any of the methods described herein, the subject is a participant in a clinical trial. In some embodiments of any of the methods described herein, the subject has been previously administered a pharmaceutical composition and the different pharmaceutical composition was determined not to be therapeutically effective. In some embodiments of any of the methods described herein, the subject has been previously administered an anti-TNFα agent and the anti-TNFα agent was determined not to be therapeutically effective

The term “administration” or “administering” refers to a method of providing a dosage of a pharmaceutical composition or a compound to an invertebrate or a vertebrate, including a fish, a bird and a mammal (e.g., a human). In some aspects, administration is performed, e.g., orally, intravenously, subcutaneously, intranasally, transdermally, intraperitoneally, intramuscularly, intrapulmonarilly, intralymphatic, topically, intraocularly, vaginally, rectally, intrathecally, or intracystically. The method of administration can depend on various factors, e.g., the site of the disease, the severity of the disease, and the components of the pharmaceutical composition.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The phrase “an elevated level” or “an increased level” as used herein can be an increase of 1.1× to 100×, or higher (e.g., 1.1×, 1.2×, 1.4×, 1.6×, 1.8×, 2×, 2.2×, 2.4×, 2.5×, 2.6×, 2.8×, 3×, 3.2×, 3.4×, 3.5×, 3.6×, 3.8×, 4×, 4.2×, 4.4×, 4.5×, 4.6×, 4.8×, 5×, 5.5×, 6×, 6.5×, 7×, 7.5×, 8×, 8.5×, 9×, 9.5×, 10×, 10.5×, 11×, 11.5×, 12×, 12.5×, 13×, 13.5×, 14×, 14.5×, 15×, 15.5×, 16×, 16.5×, 17×, 17.5×, 18×, 18.5×, 19×, 19.5×, 20×, 21×, 22×, 23×, 24×, 25×, 26×, 27×, 28×, 29×, 30×, 32×, 34×, 36×, 38×, 40×, 42×, 44×, 45×, 46×, 48×, 50×, 52×, 54×, 55×, 56×, 58×, 60×, 62×, 64×, 65×, 66×, 68×, 70×, 72×, 74×, 75×, 76×, 78×, 80×, 82×, 84×, 85×, 86×, 88×, 90×, 92×, 94×, 95×, 96×, 98×, 100×, 102×, 104×, 105×, 106×, 108×, 110×, 112×, 114×, 115×, 116×, 118×, 120×, 122×, 124×, 126×, 128×, 130×, 132×, 134×, 135×, 136×, 138×, 140×, 142×, 144×, 145×, 146×, 148×, 150×, 160×, 170×, 180×, 190×, 195×, or 200×) e.g., as compared to a reference level (e.g., any of the exemplary reference levels described herein). In some aspects, “an elevated level” or “an increased level” can be an increase of at least 1% (e.g., at least 2%, at least 4, at least 6%, at least 8%, at least 10%, at least 12%, at least 14%, at least 16%, at least 18%, at least 20%, at least 22%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 110%, at least 120%, at least 130%, at least 140%, at least 150%, at least 160%, at least 170%, at least 180%, at least 190%, at least 200%, at least 220%, at least 250%, at least 280%, at least 300%, at least 320%, at least 350%, at least 380%, at least 400%, at least 420%, at least 450%, at least 480%, at least 500%, at least 600%, at least 700%, at least 800%, at least 900%, at least 1000%, between 1% and 1000%, between 1% and 900%, between 1% and 800%, between 1% and 700%, between 1% and 600%, between 1% and 500%, between 1% and 450%, between 1% and 400%, between 1% and 350%, between 1% and 300%, between 1% and 250%, between 1% and 200%, between 1% and 180%, between 1% and 160%, between 1% and 140%, between 1% and 120%, between 1% and 100%, between 1% and 95%, between 1% and 90%, between 1% and 85%, between 1% and 80%, between 1% and 75%, between 1% and 70%, between 1% and 65%, between 1% and 60%, between 1% and 55%, between 1% and 50%, between 1% and 45%, between 1% and 40%, between 1% and 35%, between 1% and 30%, between 1% and 25%, between 1% and 20%, between 1% and 15%, between 1% and 10%, between 1% and 5%, between 5% and 1000%, between 5% and 900%, between 5% and 800%, between 5% and 700%, between 5% and 600%, between 5% and 500%, between 5% and 450%, between 5% and 400%, between 5% and 350%, between 5% and 300%, between 5% and 250%, between 5% and 200%, between 5% and 180%, between 5% and 160%, between 5% and 140%, between 5% and 120%, between 5% and 100%, between 5% and 95%, between 5% and 90%, between 5% and 85%, between 5% and 80%, between 5% and 75%, between 5% and 70%, between 5% and 65%, between 5% and 60%, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%, between 10% and 1000%, between 10% and 900%, between 10% and 800%, between 10% and 700%, between 10% and 600%, between 10% and 500%, between 10% and 450%, between 10% and 400%, between 10% and 350%, between 10% and 300%, between 10% and 250%, between 10% and 200%, between 10% and 180%, between 10% and 160%, between 10% and 140%, between 10% and 120%, between 10% and 100%, between 10% and 95%, between 10% and 90%, between 10% and 85%, between 10% and 80%, between 10% and 75%, between 10% and 70%, between 10% and 65%, between 10% and 60%, between 10% and 55%, between 10% and 50%, between 10% and 45%, between 10% and 40%, between 10% and 35%, between 10% and 30%, between 10% and 25%, between 10% and 20%, between 10% and 15%, between 20% and 1000%, between 20% and 900%, between 20% and 800%, between 20% and 700%, between 20% and 600%, between 20% and 500%, between 20% and 450%, between 20% and 400%, between 20% and 350%, between 20% and 300%, between 20% and 250%, between 20% and 200%, between 20% and 180%, between 20% and 160%, between 20% and 140%, between 20% and 120%, between 20% and 100%, between 20% and 95%, between 20% and 90%, between 20% and 85%, between 20% and 80%, between 20% and 75%, between 20% and 70%, between 20% and 65%, between 20% and 60%, between 20% and 55%, between 20% and 50%, between 20% and 45%, between 20% and 40%, between 20% and 35%, between 20% and 30%, between 20% and 25%, between 30% and 1000%, between 30% and 900%, between 30% and 800%, between 30% and 700%, between 30% and 600%, between 30% and 500%, between 30% and 450%, between 30% and 400%, between 30% and 350%, between 30% and 300%, between 30% and 250%, between 30% and 200%, between 30% and 180%, between 30% and 160%, between 30% and 140%, between 30% and 120%, between 30% and 100%, between 30% and 95%, between 30% and 90%, between 30% and 85%, between 30% and 80%, between 30% and 75%, between 30% and 70%, between 30% and 65%, between 30% and 60%, between 30% and 55%, between 30% and 50%, between 30% and 45%, between 30% and 40%, between 30% and 35%, between 40% and 1000%, between 40% and 900%, between 40% and 800%, between 40% and 700%, between 40% and 600%, between 40% and 500%, between 40% and 450%, between 40% and 400%, between 40% and 350%, between 40% and 300%, between 40% and 250%, between 40% and 200%, between 40% and 180%, between 40% and 160%, between 40% and 140%, between 40% and 120%, between 40% and 100%, between 40% and 95%, between 40% and 90%, between 40% and 85%, between 40% and 80%, between 40% and 75%, between 40% and 70%, between 40% and 65%, between 40% and 60%, between 40% and 55%, between 40% and 50%, between 40% and 45%, between 50% and 1000%, between 50% and 900%, between 50% and 800%, between 50% and 700%, between 50% and 600%, between 50% and 500%, between 50% and 450%, between 50% and 400%, between 50% and 350%, between 50% and 300%, between 50% and 250%, between 50% and 200%, between 50% and 180%, between 50% and 160%, between 50% and 140%, between 50% and 120%, between 50% and 100%, between 50% and 95%, between 50% and 90%, between 50% and 85%, between 50% and 80%, between 50% and 75%, between 50% and 70%, between 50% and 65%, between 50% and 60%, between 50% and 55%, between 60% and 1000%, between 60% and 900%, between 60% and 800%, between 60% and 700%, between 60% and 600%, between 60% and 500%, between 60% and 450%, between 60% and 400%, between 60% and 350%, between 60% and 300%, between 60% and 250%, between 60% and 200%, between 60% and 180%, between 60% and 160%, between 60% and 140%, between 60% and 120%, between 60% and 100%, between 60% and 95%, between 60% and 90%, between 60% and 85%, between 60% and 80%, between 60% and 75%, between 60% and 70%, between 60% and 65%, between 70% and 1000%, between 70% and 900%, between 70% and 800%, between 70% and 700%, between 70% and 600%, between 70% and 500%, between 70% and 450%, between 70% and 400%, between 70% and 350%, between 70% and 300%, between 70% and 250%, between 70% and 200%, between 70% and 180%, between 70% and 160%, between 70% and 140%, between 70% and 120%, between 70% and 100%, between 70% and 95%, between 70% and 90%, between 70% and 85%, between 70% and 80%, between 70% and 75%, between 80% and 1000%, between 80% and 900%, between 80% and 800%, between 80% and 700%, between 80% and 600%, between 80% and 500%, between 80% and 450%, between 80% and 400%, between 80% and 350%, between 80% and 300%, between 80% and 250%, between 80% and 200%, between 80% and 180%, between 80% and 160%, between 80% and 140%, between 80% and 120%, between 80% and 100%, between 80% and 95%, between 80% and 90%, between 80% and 85%, between 90% and 1000%, between 90% and 900%, between 90% and 800%, between 90% and 700%, between 90% and 600%, between 90% and 500%, between 90% and 450%, between 90% and 400%, between 90% and 350%, between 90% and 300%, between 90% and 250%, between 90% and 200%, between 90% and 180%, between 90% and 160%, between 90% and 140%, between 90% and 120%, between 90% and 100%, between 90% and 95%, between 100% and 1000%, between 100% and 900%, between 100% and 800%, between 100% and 700%, between 100% and 600%, between 100% and 500%, between 100% and 450%, between 100% and 400%, between 100% and 350%, between 100% and 300%, between 100% and 250%, between 100% and 200%, between 100% and 180%, between 100% and 160%, between 100% and 140%, between 100% and 120%, between 120% and 1000%, between 120% and 900%, between 120% and 800%, between 120% and 700%, between 120% and 600%, between 120% and 500%, between 120% and 450%, between 120% and 400%, between 120% and 350%, between 120% and 300%, between 120% and 250%, between 120% and 200%, between 120% and 180%, between 120% and 160%, between 120% and 140%, between 140% and 1000%, between 140% and 900%, between 140% and 800%, between 140% and 700%, between 140% and 600%, between 140% and 500%, between 140% and 450%, between 140% and 400%, between 140% and 350%, between 140% and 300%, between 140% and 250%, between 140% and 200%, between 140% and 180%, between 140% and 160%, between 160% and 1000%, between 160% and 900%, between 160% and 800%, between 160% and 700%, between 160% and 600%, between 160% and 500%, between 160% and 450%, between 160% and 400%, between 160% and 350%, between 160% and 300%, between 160% and 250%, between 160% and 200%, between 160% and 180%, between 180% and 1000%, between 180% and 900%, between 180% and 800%, between 180% and 700%, between 180% and 600%, between 180% and 500%, between 180% and 450%, between 180% and 400%, between 180% and 350%, between 180% and 300%, between 180% and 250%, between 180% and 200%, between 200% and 1000%, between 200% and 900%, between 200% and 800%, between 200% and 700%, between 200% and 600%, between 200% and 500%, between 200% and 450%, between 200% and 400%, between 200% and 350%, between 200% and 300%, between 200% and 250%, between 250% and 1000%, between 250% and 900%, between 250% and 800%, between 250% and 700%, between 250% and 600%, between 250% and 500%, between 250% and 450%, between 250% and 400%, between 250% and 350%, between 250% and 300%, between 300% and 1000%, between 300% and 900%, between 300% and 800%, between 300% and 700%, between 300% and 600%, between 300% and 500%, between 300% and 450%, between 300% and 400%, between 300% and 350%, between 350% and 1000%, between 350% and 900%, between 350% and 800%, between 350% and 700%, between 350% and 600%, between 350% and 500%, between 350% and 450%, between 350% and 400%, between 400% and 1000%, between 400% and 900%, between 400% and 800%, between 400% and 700%, between 400% and 600%, between 400% and 500%, between 400% and 450%, about 450% to about 500%, between 500% and 1000%, between 500% and 900%, between 500% and 800%, between 500% and 700%, between 500% and 600%, between 600% and 1000%, between 600% and 900%, between 600% and 800%, between 600% and 700%, between 700% and 1000%, between 700% and 900%, between 700% and 800%, between 800% and 1000%, between 800% and 900%, or between 900% and 1000%), e.g., as compared to a reference level (e.g., any of the exemplary reference levels described herein).

The term “NLRP3 inflammasome activity” means direct activity of an NLRP3 inflammasome in a mammalian cell (e.g., caspase-1 cleavage activity, secretion of IL-18, and secretion of IL-14); an upstream activity or mutation (e.g., any of the exemplary mutations or single nucleotide polymorphisms described herein) in a mammalian cell that results in increased NLRP3 inflammasome activity in the mammalian cell (e.g., increased expression of one or more of lipocalin-2 protein, lipocalin-2 mRNA, S100A8 protein, S100A8 mRNA, S100A9 protein, and S100A9 mRNA, e.g., as compared to any of the exemplary reference levels described herein; detection of any of the exemplary types of gain-of-function or loss-of-function mutations, or single nucleotide polymorphisms described herein); and/or an increased downstream activity of an NLRP3 inflammasome activity in a mammalian cell (e.g., increased expression of one or more of increased expression of one or more of CRP protein, CRP mRNA, SAA protein, SAA mRNA, HP protein, HP mRNA, ceruloplasmin protein, ceruloplasmin mRNA, IL-6 protein, IP-6 mRNA, calprotectin (S100A8) protein, calprotectin (S100A8) mRNA, IL-8 protein, IL-8 mRNA, leukotriene B4 protein, leukotriene B4 mRNA, myeloperoxidase protein, and myeloperoxidase mRNA, e.g., as compared to any of the exemplary reference levels described herein). Non-limiting examples of human protein and human cDNA sequences for CRP protein, CRP mRNA, SAA protein, SAA mRNA, HP protein, HP mRNA, ceruloplasmin protein, ceruloplasmin mRNA, IL-6 protein, IP-6 mRNA, calprotectin (S100A8) protein, calprotectin (S100A8) mRNA, IL-8 protein, IL-8 mRNA, leukotriene B4 protein, leukotriene B4 mRNA, myeloperoxidase protein, and myeloperoxidase mRNA are shown below.

The sequences characterized by the Sequences ID NO: 1-34 are listed below and are being submitted in a separate and machine readable file.
Human C-Reactive Protein (CRP) Transcript Variant 1 cDNA (SEQ ID NO: 1),
Human C-Reactive Protein (CRP) Transcript Variant 1 protein (SEQ ID NO: 2)
Human C-Reactive Protein (CRP) Transcript Variant 2 cDNA (SEQ ID NO: 3)
Human C-Reactive Protein (CRP) Transcript Variant 2 protein (SEQ ID NO: 4)
Human C-Reactive Protein (CRP) Transcript Variant 3 cDNA (SEQ ID NO: 5)
Human C-Reactive Protein (CRP) Transcript Variant 3 protein (SEQ ID NO: 6)
Human Serum amyloid A1 (SAA) Transcript Variant 1 cDNA (SEQ ID NO: 7)
Human Serum amyloid A1 (SAA) Transcript Variant 1 protein (SEQ ID NO: 8)
Human Serum amyloid A1 (SAA) Transcript Variant 2 cDNA (SEQ ID NO: 9)
Human Serum amyloid A1 (SAA) Transcript Variant 2 protein (SEQ ID NO: 10)
Human Serum amyloid A1 (SAA) Transcript Variant 3 cDNA (SEQ ID NO: 11)
Human Serum amyloid A1 (SAA) Transcript Variant 3 protein (SEQ ID NO: 12)
Human Serum amyloid A1 (SAA) Transcript Variant 1 cDNA (SEQ ID NO: 13)
Human Serum amyloid A1 (SAA) Transcript Variant 1 protein (SEQ ID NO: 14)
Human Serum amyloid A1 (SAA) Transcript Variant 2 cDNA (SEQ ID NO: 15)
Human Serum amyloid A1 (SAA) Transcript Variant 2 protein (SEQ ID NO: 16)
Human Serum amyloid A1 (SAA) Transcript Variant 3 cDNA (SEQ ID NO: 17)
Human Serum amyloid A1 (SAA) Transcript Variant 3 protein (SEQ ID NO: 18)
Human Ceruloplasmin (CP) Transcript Variant 1 cDNA (SEQ ID NO: 19)
Human Ceruloplasmin (CP) Transcript Variant 1 protein (SEQ ID NO: 20)
Human Interleukin 6 (IL-6) Transcript Variant 1 cDNA (SEQ ID NO: 21),
Human Interleukin 6 (IL-6) Transcript Variant 1 protein (SEQ ID NO: 22),
Human Interleukin 6 (IL-6) Transcript Variant 2 cDNA (SEQ ID NO: 23),
Human Interleukin 6 (IL-6) Transcript Variant 2 protein (SEQ ID NO: 24),
Human Interleukin 8 (IL-8) Transcript Variant 1 cDNA (SEQ ID NO: 25),
Human Interleukin 8 (IL-8) Transcript Variant 1 protein (SEQ ID NO: 26),
Human Interleukin 8 (IL-8) Transcript Variant 2 cDNA (SEQ ID NO: 27),
Human Interleukin 8 (IL-8) Transcript Variant 2 protein (SEQ ID NO: 28),
Human Leukotriene B4 receptor (LTB4R) Transcript Variant 1 cDNA (SEQ ID NO: 29),
Human Leukotriene B4 receptor (LTB4R) Transcript Variant 1 protein (SEQ ID NO: 30),
Human Leukotriene B4 receptor (LTB4R) Transcript Variant 2 cDNA (SEQ ID NO: 31),
Human Leukotriene B4 receptor (LTB4R) Transcript Variant 2 protein (SEQ ID NO: 32),
Human Myeloperoxidase (MPO) Transcript Variant 1 cDNA (SEQ ID NO: 33),
Human Myeloperoxidase (MPO) Transcript Variant 1 protein (SEQ ID NO: 34)

NLRP3 inflammasome activity can be detected, e.g., by determining the level of expression of one or more of NLRP3, ASC, CASP1, LCN2, IL-18, IL-10, S100A8, and S100A9 in a mammalian cell; detection of a gain-of-function mutation in a NLRP3 gene (e.g., a NLRP3 protein having a Q705K amino acid substitution, a T350M amino acid substitution, a R262M amino acid substitution, a A441V amino acid substitution, a V200M amino acid substitution, an E629G amino acid substitution, a L355P amino acid substitution, a R260W amino acid substitution, a G571R amino acid substitution, a A354V amino acid substitution, a D305N amino acid substitution, a F311S amino acid substitution, a R920Q amino acid substitution, or a D21H amino acid substitution), each numbered according to the mature NLRP3 protein sequence of SEQ ID NO: 35; detection of one or more of a loss-of-function mutation in one or more of a CARD8 gene (e.g., a C allele at r52043211); detection of a T allele at rs3024505 flanking IL10 gene; detection of a R620W amino acid substitution in PTPN22; detection of a C allele at rs478582 in the PTPN2 gene; detection of a G allele at rs713875 in the MTMR3 gene; detection of a C allele at rs1042058 in the TPL2 gene; and detection of a ATG16L1 gene that encodes a ATG16L1 protein having a T300A amino acid substitution. Methods of detecting a level of each of these exemplary types of NLRP3 inflammasome activity are described herein. Additional examples of NLRP3 inflammasome activities are known in the art, as well as methods for detecting a level of the same.

As used herein, “gain-of-function mutation” refers to one or more nucleotide substitutions, deletions, and/or insertions in a gene that results in: an increase in the level of expression of the encoded protein as compared to the level of the expression by the corresponding wildtype gene, and/or the expression of a protein encoded by the gene that has one or more increased activities in a mammalian cell as compared to the version of the protein encoded by the corresponding wildtype gene.

As used herein, “loss-of-function mutation” refers to one or more nucleotide substitutions, deletions, and/or insertions in a gene that results in: a decrease in the level of expression of the encoded protein as compared to the level of the expression by the corresponding wildtype gene, and/or the expression of a protein encoded by the gene that has one or more decreased activities in a mammalian cell as compared to the version of the protein encoded by the corresponding wildtype gene.

As used herein, the phrase “resistance to an anti-TNFα agent” refers to a reduced or decreased level of sensitivity to treatment with an anti-TNFα agent in a subject (e.g., as compared to a similar subject or as compared to the level of sensitivity to the anti-TNFα agent at an earlier time point). For example, resistance to an anti-TNFα in a subject can be observed by a physician, e.g., by observing the requirement of a increasing dosage amounts of an anti-TNFα agent over time in order to achieve the same therapeutic effect in a subject, observing the requirement for an increased number of doses and/or an increased frequency of doses of an anti-TNFα agent over time in order to achieve the same therapeutic effect in a subject, a decrease in the observed therapeutic response to treatment with the same dosage of an anti-TNFα agent over time, or an observed progression of disease or disease relapse in a subject administered an anti-TNFα agent.

As used herein, the phrase “beneficial response” refers to a therapeutic benefit and/or an improved clinical outcome to a subject suffering from a TNFα-associated disease from or as a result of the treatment with a NLRP3 antagonist. In some embodiments, a beneficial response is a cellular response.

The terms “hydrogen” and “H” are used interchangeably herein.

The term “halo” refers to fluoro (F), chloro (Cl), bromo (Br), or iodo (I).

The term “alkyl” refers to a hydrocarbon chain that may be a straight chain or branched chain, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl.

The term “haloalkyl” refers to an alkyl, in which one or more hydrogen atoms is/are replaced with an independently selected halo.

The term “alkoxy” refers to an —O-alkyl radical (e.g., —OCH3).

The term “carbocyclic ring” as used herein includes an aromatic or nonaromatic cyclic hydrocarbon group having 3 to 10 carbons, such as 3 to 8 carbons, such as 3 to 7 carbons, which may be optionally substituted. Examples of carbocyclic rings include five-membered, six membered, and seven-membered carbocyclic rings.

The term “heterocyclic ring” refers to an aromatic or nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclic rings include five-membered, six membered, and seven-membered heterocyclic rings.

The term “cycloalkyl” as used herein includes an aromatic or nonaromatic cyclic hydrocarbon radical having 3 to 10 carbons, such as 3 to 8 carbons, such as 3 to 7 carbons, wherein the cycloalkyl group which may be optionally substituted. Examples of cycloalkyls include five membered, six-membered, and seven-membered rings. Examples include cyclopropyl, cyclobutyl, cyclopentyl, cyclopentenyl, cyclohexyl, cyclohexenyl, cycloheptyl, and cyclooctyl.

The term “heterocycloalkyl” refers to an aromatic or nonaromatic 5-8 membered monocyclic, 8-12 membered bicyclic, or 11-14 membered tricyclic ring system radical having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocycloalkyls include five-membered, six membered, and seven-membered heterocyclic rings. Examples include piperazinyl, pyrrolidinyl, dioxanyl, morpholinyl, tetrahydrofuranyl, and the like.

The term “hydroxy” refers to an OH group.

The term “amino” refers to an NH2 group.

The term “oxo” refers to O. By way of example, substitution of a CH2 a group with oxo gives a C═O group.

A gut-targeted NLRP3 antagonist is any NLRP3 antagonist, which has the following characteristics:
potent NLRP3 inhibitors, e.g, <1 micromolar in cellular assay systems, as assessed for example using a nigericin-stimulated IL-1β secretion assay in THP-1 cells
with low permeability as assessed in an MDCK assay (Pefflux <2×10\hat{ }6 cm/sec),
poor systemic bioavailability, as assessed in a rat or mouse PK experiment,
high levels of compound in the colon, for example 500 nM or more of the NLRP3 antagonist in the colon, as assessed using tissue distribution models.

As used herein, the terms “patient” or “subject” refer to a mammalian organism, preferably a human being, who is diseased with the condition (i.e. disease or disorder) of interest and who would benefit from the treatment.

As used herein, the term “prevent”, “preventing” or “prevention” in connection to a disease or disorder refers to the prophylactic treatment of a subject who is at risk of developing a condition (e.g., specific disease or disorder or clinical symptom thereof) resulting in a decrease in the probability that the subject will develop the condition.

As used herein, the term “treat”, “treating” or “treatment” of any disease or disorder refers in one embodiment to ameliorating the disease or disorder (i.e. slowing or arresting or reducing the development of the disease or at least one of the clinical symptoms or pathological features thereof). In another embodiment “treat”, “treating” or “treatment” refers to alleviating or ameliorating at least one physical parameter or pathological features of the disease, e.g. including those, which may not be discernible by the subject. In yet another embodiment, “treat”, “treating” or “treatment” refers to modulating the disease or disorder, either physically, (e.g. stabilization of at least one discernible or non-discernible symptom), physiologically (e.g. stabilization of a physical parameter) or both. In yet another embodiment, “treat”, “treating” or “treatment” refers to preventing or delaying the onset or development or progression of the disease or disorder, or of at least one symptoms or pathological features associated thereof. In yet another embodiment, “treat”, “treating” or “treatment” refers to preventing or delaying progression of the disease to a more advanced stage or a more serious condition.

As used herein, the term “therapeutically effective amount” refers to an amount of the compound of the invention, e.g. a NLRP3 antagonist as herein defined, e.g. in free form or as a stereoisomer, an enantiomer, a pharmaceutically acceptable salt, solvate, prodrug, ester thereof and/or an amino acid conjugate thereof), or cenicriviroc (in free form or as a pharmaceutically acceptable salt, solvate, prodrug, and/or ester thereof, e.g. in free form or as a pharmaceutically acceptable salt thereof), which is sufficient to achieve the stated effect. Accordingly, a therapeutically effective amount used for the treatment or prevention of a liver disease or disorder as hereinabove defined is an amount sufficient for the treatment or prevention of such a disease or disorder.

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

Other features and advantages of the invention will be apparent from the following detailed description and figures, and from the claims.

### DESCRIPTION OF THE DRAWINGS

FIG. 1: Expression levels of RNA encoding NLRP3 in Crohn's Disease patients who are responsive and non-responsive to infliximab.

FIG. 2: Expression levels of RNA encoding IL-1β in Crohn's Disease patients who are responsive and non-responsive to infliximab.

FIG. 3: Expression levels of RNA encoding NLRP3 in Ulcerative Colitis (UC) patients who are responsive and non-responsive to infliximab.

FIG. 4: Expression levels of RNA encoding IL-1β in Ulcerative Colitis (UC) patients who are responsive and non-responsive to infliximab.

### DETAILED DESCRIPTION

The present inventions are based on the discovery that specific genetic mutations and/or protein/mRNA expression profiles correlate with increased NLRP3 inflammasome activity and expression, and can be used to identify subjects who are more likely to have a therapeutic response to treatment with an NLRP3 antagonist. In view of these discoveries, provided herein are methods of treating a subject that include: (a) identifying a subject having a cell that has an elevated level of NLRP3 inflammasome activity and/or expression as compared to a reference level; and (b) administering to the identified subject a therapeutically effective amount of an NLRP3 antagonist or a pharmaceutically acceptable salt, solvate, or co-crystal thereof. Also provided are methods of treating a subject that include administering a therapeutically effective amount of an NLPR3 antagonist or a pharmaceutically acceptable salt, solvate, or co-crystal thereof to a subject identified as having a cell that has an elevated level of NLRP3 inflammasome activity and/or expression as compared to a reference level.

Also provided are methods of selecting a treatment for a subject that include: (a) identifying a subject having a cell that has an elevated level of NLRP3 inflammasome activity and/or expression as compared to a reference level; and (b) selecting for the identified subject a treatment comprising a therapeutically effective amount of an NLRP3 antagonist or a pharmaceutically acceptable salt, solvate, or co-crystal thereof. Also provided are methods of selecting a treatment for a subject that include selecting a treatment comprising a therapeutically effective amount of an NLRP3 antagonist or a pharmaceutically acceptable salt, solvate, or co-crystal thereof for a subject identified as having a cell that has an elevated level of NLRP3 inflammasome activity and/or expression as compared to a reference level.

Also provided herein are methods of selecting a subject for treatment that include: (a) identifying a subject having a cell that has an elevated level of NLRP3 inflammasome activity and/or expression as compared to a reference level; and (b) selecting an identified subject for treatment with a therapeutically effective amount of an NLRP3 antagonist or a pharmaceutically acceptable salt, solvate, or co-crystal thereof. Also provided are methods selecting a subject for treatment that include selecting a subject identified as having a cell that has an elevated level of NLRP3 inflammasome activity and/or expression as compared to a reference level, for treatment with a therapeutically effective amount of an NLRP3 antagonist or a pharmaceutically acceptable salt, solvate, or co-crystal thereof.

Also provided herein are methods of selecting a subject for participation in a clinical trial that include: identifying a subject having a cancer cell that has an elevated level of NLRP3 inflammasome activity and/or expression as compared to a reference level; and selecting the identified subject for participation in a clinical trial that comprises administration of a therapeutically effective amount of a NLRP3 antagonist or a pharmaceutically acceptable salt, solvate, or co-crystal thereof. Also provided herein are methods of selecting a subject for participation in a clinical trial that include selecting a subject identified as having a cell that has an elevated level of NLRP3 inflammasome activity and/or expression as compared to a reference level for participation in a clinical trial that comprises administration of a therapeutically effective amount of an NLRP3 antagonist or a pharmaceutically acceptable salt, solvate, or co-crystal thereof.

The present invention is also based on the discovery that a NLRP3 antagonist (e.g., any of the NLRP3 antagonists described herein) can reduce resistance to an anti-TNFα agent (e.g., any of the exemplary resistances to an anti-TNFα agent described herein or known in the art) in a subject. In view of these discoveries, provided herein are methods of treating a subject in need thereof that include (a) identifying a subject having resistance to an anti-TNFα agent (e.g., any of the exemplary resistances to an anti-TNFα agent described herein or known in the art) as compared to a reference level (e.g., any of the exemplary reference levels of NLRP3 inflammasome activity described herein or known in the art); and (b) administering a treatment comprising a therapeutically effective amount of an NLRP3 antagonist or a pharmaceutically acceptable salt, solvate, or co-crystal thereof to the identified subject. Also provided herein are methods of treating a subject identified as having resistance to an anti-TNFα agent (e.g., any of the exemplary resistances to an anti-TNFα agent described herein or known in the art) with an NLRP3 antagonist (e.g., any of the exemplary NLRP3 antagonists described herein) or a pharmaceutically acceptable salt, solvate, or co-crystal thereof

Non-liming aspects of these methods are described below, and can be used in any combination without limitation. Additional aspects of these methods are known in the art.

In one embodiment, the present invention relates to an NLRP3 antagonist for use in the treatment or the prevention of a condition mediated by TNF-α, in particular a gut disease or disorder, in a patient in need thereof, wherein the NLRP3 antagonist is administered to said patient at a therapeutically effective amount.

In one embodiment, the present invention relates to an NLRP3 antagonist for use in the treatment or the prevention of a condition, in particular a gut disease or disorder, in a patient in need thereof wherein the NLRP3 antagonist is administered to said patient at a therapeutically effective amount.

In one embodiment, the present invention relates to an NLRP3 antagonist for use in the treatment, stabilization or lessening the severity or progression of gut disease or disorder, in a patient in need thereof wherein the NLRP3 antagonist is administered to said patient at a therapeutically effective amount.

In one embodiment, the present invention relates to an NLRP3 antagonist for use in the slowing, arresting, or reducing the development of a gut disease or disorder, in a patient in need thereof wherein the NLRP3 antagonist is administered to said patient at a therapeutically effective amount.

In one embodiment, the present invention relates to an NLRP3 antagonist for use according to above listed embodiments wherein the NLRP3 antagonist is a gut-targeted NLRP3 antagonist.

In one embodiment, the present invention relates to an NLRP3 antagonist for use according to any of the above embodiments, wherein the gut disease is IBD.

In one embodiment, the present invetion relates to an NLRP3 antagonist for use according to any of the above embodiments, wherein the gut disease is UC or CD.

In one embodiment, the present invetion relates to a method for the treatment or the prevention of a condition mediated by TNF-α, in particular a gut disease or disorder, in a patient in need thereof, comprising administering to said patient a therapeutically effective amount of a gut-targeted NLRP3 antagonist.

In one embodiment, the present invention relates to a method for the treatment or the prevention of a condition, in particular a gut disease or disorder, in a patient in need thereof, comprising administering to said patient a therapeutically effective amount of a gut-targeted NLRP3 antagonist.

In one embodiment, the present invention relates to a method for the treatment, stabilization or lessening the severity or progression of gut disease or disorder, in a patient in need thereof comprising administering to said patient a therapeutically effective amount of a gut-targeted NLRP3 antagonist.

In one embodiment, the present invention relates to a method for slowing, arresting, or reducing the development of a gut disease or disorder, in a patient in need thereof comprising administering to said patient a therapeutically effective amount of a gut-targeted NLRP3 antagonist.

In one embodiment, the present invention relates to a method according to any of the above embodiments, wherein the gut disease is IBD.

In one embodiment, the present invention relates to a method as described herein wherein the gut disease is UC or CD.

An exemplary sequence of human NLRP3 protein is being submitted in a separate and machine readable file. Mature Human NLRP3 Protein (SEQ ID NO: 35).

### Methods of Treating

Provided herein are methods of treating a subject (e.g., any of the exemplary subjects described herein) that include: (a) identifying a subject having a cell that has an elevated level (e.g., an increase of 1.1× to 100× or higher, or any of the subranges of this range described herein) of NLRP3 inflammasome activity and/or expression as compared to a reference level; and (b) administering to the identified subject a therapeutically effective amount of an NLRP3 antagonist (e.g., any of the exemplary NLRP3 antagonists described herein) or a pharmaceutically acceptable salt, solvate, or co-crystal thereof.

Also provided herein are methods of treating a subject (e.g., any of the exemplary subjects described herein) that include: administering a therapeutically effective amount of an NLPR3 antagonist (e.g., any of the exemplary NLRP3 antagonists described herein) or a pharmaceutically acceptable salt, solvate, or co-crystal thereof to a subject identified as having a cell that has an elevated level (e.g., an increase of 1.1× to 100× or higher, or any of the subranges of this range described herein) of NLRP3 inflammasome activity and/or expression as compared to a reference level.

In some embodiments of any of the methods described herein, the NLRP3 inflammasome activity is secretion of IL-18. In some embodiments of any of the methods described herein, the NLRP3 inflammasome activity is secretion of IL-10. In some embodiments of any of the methods described herein, the NLRP3 inflammasome activity is caspase-1 activity. In some embodiments of any of the methods described herein, the NLRP3 inflammasome activity is the level of lipocalin-2 (LCN2). In some embodiments of any of the methods described herein, the NLRP3 inflammasome activity is the level of S100A8. In some embodiments of any of the methods described herein, the NLRP3 inflammasome activity is the level of S100A9.

In some embodiments of any of the methods described herein, the identifying a subject having a cell that has an elevated level of NLRP3 inflammasome activity comprises detecting a gain-of-function mutation in an NLRP3 gene (e.g., a NLRP3 protein having a Q705K amino acid substitution, a T350M amino acid substitution, a R262M amino acid substitution, a A441V amino acid substitution, a V200M amino acid substitution, an E629G amino acid substitution, a L355P amino acid substitution, a R260W amino acid substitution, a G571R amino acid substitution, a A354V amino acid substitution, a D305N amino acid substitution, a F311S amino acid substitution, a R920Q amino acid substitution, or a D21H amino acid substitution, each numbered according to the mature NLRP3 protein sequence of SEQ ID NO: 35) in a cell from the subject.

In some embodiments of any of the methods described herein, the subject identified as having a cell that has an elevated level of NLRP3 inflammasome activity has been determined to have a cell having a gain-of-function mutation in an NLRP3 gene (e.g., a NLRP3 protein having a Q705K amino acid substitution, a T350M amino acid substitution, a R262M amino acid substitution, a A441V amino acid substitution, a V200M amino acid substitution, an E629G amino acid substitution, a L355P amino acid substitution, a R260W amino acid substitution, a G571R amino acid substitution, a A354V amino acid substitution, a D305N amino acid substitution, a F311S amino acid substitution, a R920Q amino acid substitution, or a D21H amino acid substitution, each numbered according to the mature NLRP3 protein sequence of SEQ ID NO: 35).

In some embodiments of any of the methods described herein, the identifying a subject having a cell that has an elevated level of NLRP3 inflammasome activity comprises detecting a loss-of-function mutation in a CARD8 gene (e.g., a C allele at rs2043211) in a cell from the subject.

In some embodiments of any of the methods described herein, the subject identified as having a cell that has an elevated level of NLRP3 inflammasome activity has been determined to have a cell having a loss-of-function mutation in a CARD8 gene (e.g., a C allele at rs2043211).

In some embodiments of any of the methods described herein, the identifying a subject having a cell that has an elevated level of NLRP3 inflammasome activity comprises detecting a gain-of-function mutation in an NLRP3 gene (e.g., a NLRP3 protein having a Q705K amino acid substitution, a T350M amino acid substitution, a R262M amino acid substitution, a A441V amino acid substitution, a V200M amino acid substitution, an E629G amino acid substitution, a L355P amino acid substitution, a R260W amino acid substitution, a G571R amino acid substitution, a A354V amino acid substitution, a D305N amino acid substitution, a F311S amino acid substitution, a R920Q amino acid substitution, or a D21H amino acid substitution, each numbered according to the mature NLRP3 protein sequence of SEQ ID NO: 35) and a loss-of-function mutation in a CARD8 gene (e.g., a C allele at rs2043211) in a cell from the subject.

In some embodiments of any of the methods described herein, the subject identified as having a cell that has an elevated level of NLRP3 inflammasome activity has been determined to have a cell having a gain-of-function mutation in an NLRP3 gene (e.g., a NLRP3 protein having a Q705K amino acid substitution, a T350M amino acid substitution, a R262M amino acid substitution, a A441V amino acid substitution, a V200M amino acid substitution, an E629G amino acid substitution, a L355P amino acid substitution, a R260W amino acid substitution, a G571R amino acid substitution, a A354V amino acid substitution, a D305N amino acid substitution, a F311S amino acid substitution, a R920Q amino acid substitution, or a D21H amino acid substitution, each numbered according to the mature NLRP3 protein sequence of SEQ ID NO: 35) and a loss-of-function mutation in a CARD8 gene (e.g., a C allele at rs2043211).

In some embodiments of any of the methods described herein, the identifying a subject having a cell that has an elevated level of NLRP3 inflammasome activity comprises detecting a T allele at rs3024505 flanking an IL10 gene in a cell from the subject.

In some embodiments of any of the methods described herein, the subject identified as having a cell that has an elevated level of NLRP3 inflammasome activity has been determined to have a cell having a T allele at rs3024505 flanking IL10 gene in a cell.

In some embodiments of any of the methods described herein, the identifying a subject having a cell that has an elevated level of NLRP3 inflammasome activity comprises detecting a PTPN22 gene that encodes a PTPN22 protein having a R620W amino acid substitution in a cell from the subject.

In some embodiments of any of the methods described herein, the subject identified as having a cell that has an elevated level of NLRP3 inflammasome activity has been determined to have a cell having a PTPN22 gene that encodes a PTPN22 protein having a R620W amino acid substitution.

In some embodiments of any of the methods described herein, the identifying a subject having a cell that has an elevated level of NLRP3 inflammasome activity comprises detecting a C allele at rs478582 in the PTPN2 gene in a cell from the subject.

In some embodiments of any of the methods described herein, the subject identified as having a cell that has an elevated level of NLRP3 inflammasome activity has been determined to have a cell having a C allele at rs478582 in the PTPN2 gene.

In some embodiments of any of the methods described herein, the identifying a subject having a cell that has an elevated level of NLRP3 inflammasome activity comprises detecting a G allele at rs713875 in the MTMR3 gene in a cell from the subject.

In some embodiments of any of the methods described herein, the subject identified as having a cell that has an elevated level of NLRP3 inflammasome activity has been determined to have a cell having a G allele at rs713875 in the MTMR3 gene.

In some embodiments of any of the methods described herein, the identifying a subject having a cell that has an elevated level of NLRP3 inflammasome activity comprises detecting a C allele at rs1042058 in the TPL2 gene in a cell from the subject.

In some embodiments of any of the methods described herein, the subject identified as having a cell that has an elevated level of NLRP3 inflammasome activity has been determined to have a cell having a C allele at rs1042058 in the TPL2 gene.

In some embodiments of any of the methods described herein, the identifying a subject having a cell that has an elevated level of NLRP3 inflammasome activity comprises detecting a ATG16L1 gene that encodes a ATG16L1 protein having a T300A amino acid substitution in a cell from the subject.

In some embodiments of any of the methods described herein, the subject identified as having a cell that has an elevated level of NLRP3 inflammasome activity has been determined to have a cell having a ATG16L1 gene that encodes a ATG16L1 protein having a T300A amino acid substitution.

In some embodiments of any of the methods described herein, the gain-of-function mutation in an NLRP3 gene results in the expression of a NLRP3 protein having a Q705K amino acid substitution. In some embodiments of any of the methods described herein, the loss-of-function mutation in a CARD8 gene is a C allele at rs2043211.

In some embodiments of any of the methods described herein, the identifying a subject having a cell that has an elevated level of NLRP3 inflammasome expression comprises detecting the level of one or more of NLRP3 protein, ASC protein, procaspase-1 protein, caspase-1 protein, IL-18 protein (e.g., mature or pro-IL-18 protein), IL-1β protein (e.g., mature or pro-IL-14 protein), LCN2 protein, S100A8 protein, and S100A9 protein.

In some embodiments of any of the methods described herein, the identifying a subject having a cell that has an elevated level of NLRP3 inflammasome expression comprises detecting the level of one or more of NLRP3 mRNA, ASC mRNA, and procaspase-1 mRNA, pro-IL-18 mRNA, pro-IL-13 mRNA, LCN2 mRNA, S100A8 mRNA, and S100A9 mRNA.

In some embodiments of any of the methods described herein the subject identified as having a cell that has an elevated level of NLRP3 inflammasome expression has been determined to have a cell having an elevated level of one or more of NLRP3 protein, ASC protein, procaspase-1 protein, caspase-1 protein, IL-18 protein (e.g., mature or pro-IL-18 protein), IL-1β protein (e.g., mature or pro-IL-14 protein), LCN2 protein, S100A8 protein, and S100A9 protein.

In some embodiments of any of the methods described herein the subject identified as having a cell that has an elevated level of NLRP3 inflammasome expression has been determined to have a cell having an elevated level of one or more of NLRP3 mRNA, ASC mRNA, and procaspase-1 mRNA.

In some embodiments of any of the methods described herein, the identifying a subject having a cell that has an elevated level of NLRP3 inflammasome expression comprises detecting the level of one or more of CRP protein, SAA protein, HP protein, ceruloplasmin protein, IL-6 protein (e.g., mature or pro-IL-6 protein), calprotectin (S100A8) protein, IL-8 protein (e.g., mature or pro-IL-8 protein), leukotriene B4 protein, and myeloperoxidase protein.

In some embodiments of any of the methods described herein, the identifying a subject having a cell that has an elevated level of NLRP3 inflammasome expression comprises detecting the level of one or more of CRP mRNA, SAA mRNA, HP mRNA, ceruloplasmin mRNA, pro-IL-6 mRNA, calprotectin (S100A8) mRNA, pro-IL-8 mRNA, leukotriene B4 mRNA, and myeloperoxidase mRNA.

In some embodiments of any of the methods described herein the subject identified as having a cell that has an elevated level of NLRP3 inflammasome expression has been determined to have a cell having an elevated level of one or more of CRP protein, SAA protein, HP protein, ceruloplasmin protein, IL-6 protein (e.g., mature or pro-IL-6 protein), calprotectin (S100A8) protein, IL-8 protein (e.g., mature or pro-IL-8 protein), leukotriene B4 protein, and myeloperoxidase protein.

In some embodiments of any of the methods described herein the subject identified as having a cell that has an elevated level of NLRP3 inflammasome expression has been determined to have a cell having an elevated level of one or more of CRP mRNA, SAA mRNA, HP mRNA, ceruloplasmin mRNA, pro-IL-6 mRNA, calprotectin (S100A8) mRNA, pro-IL-8 mRNA, leukotriene B4 mRNA, and myeloperoxidase mRNA.

In some embodiments of any of the methods described herein the subject has or is suspected of having Crohn's disease, inflammatory bowel disease (IBD), or other gastrointestinal, autoimmune, or autoinflammatory disorders.

In some embodiments of any of the methods described herein, the identifying a subject having a cell that has an elevated level of NLRP3 inflammasome activity comprises detecting a mutation in an NLRP3 gene that results in the expression of a NLRP3 protein having one or both of a T350M and a R262M amino acid substitution (each numbered according to the mature NLRP3 protein sequence of SEQ ID NO: 35) in a cell from the subject. In some embodiments of any of the methods described herein, the subject identified as having a cell that has an elevated level of NLRP3 inflammasome activity has been determined to have a cell having an NLRP3 gene that results in the expression of a NLRP3 protein having one or both of a T350M and a R262M amino acid substitution (each numbered according to the mature NLRP3 protein sequence of SEQ ID NO: 35). In some embodiments of these methods, the subject has or is suspected of having hereditary periodic fever.

In some embodiments of any of the methods described herein, the identifying a subject having a cell that has an elevated level of NLRP3 inflammasome activity comprises detecting a mutation in an NLRP3 gene that results in the expression of an NLRP3 protein having one or more of a A441V, a V200M, a E629G, and a L355P amino acid substitution (each numbered according to the mature NLRP3 protein sequence of SEQ ID NO: 35) in a cell from the subject. In some embodiments of any of the methods described herein, the subject identified as having a cell that has an elevated level of NLRP3 inflammasome activity has been determined to have a cell having an NLRP3 gene that results in the expression of an NLRP3 protein having one or more of a A441V, a V200M, a E629G, and a L355P amino acid substitution (each numbered according to the mature NLRP3 protein sequence of SEQ ID NO: 35). In some embodiments of these methods, the subject has or is suspected of having familial cold autoinflammatory syndrome (FCAS).

In some embodiments of any of the methods described herein, the identifying a subject having a cell that has an elevated level of NLRP3 inflammasome activity comprises detecting a mutation in an NLRP3 gene that results in the expression of an NLRP3 protein having one or more of a R260W, a G571R, and a A354V amino acid substitution (each numbered according to the mature NLRP3 protein sequence of SEQ ID NO: 35) in a cell from the subject. In some embodiments of any of the methods described herein, the subject identified as having a cell that has an elevated level of NLRP3 inflammasome activity has been determined to have a cell having an NLRP3 gene that results in the expression of an NLRP3 protein having one or more of a R260W, a G571R, and a A354V amino acid substitution (each numbered according to the mature NLRP3 protein sequence of SEQ ID NO: 35). In some of embodiments of these methods, the subject has or is suspected of having Muckle-Wells syndrome (MWS).

In some embodiments of any of the methods described herein, the identifying a subject having a cell that has an elevated level of NLRP3 inflammasome activity comprises detecting a mutation in an NLRP3 gene that results in the expression of an NLRP3 protein having one or both of a D305N and a F311S amino acid substitution (each numbered according to the mature NLRP3 protein sequence of SEQ ID NO: 35) in a cell from the subject. In some embodiments of any of the methods described herein, the subject identified as having a cell that has an elevated level of NLRP3 inflammasome activity has been determined to have a cell having an NLRP3 gene that results in the expression of an NLRP3 protein having one or both of a D305N and a F311S amino acid substitution (each numbered according to the mature NLRP3 protein sequence of SEQ ID NO: 35). In some embodiments of these methods, the subject has or is suspected of having Cinca syndrome.

In some embodiments of any of the methods described herein, the identifying a subject having a cell that has an elevated level of NLRP3 inflammasome activity comprises detecting a mutation in an NLRP3 gene that results in the expression of an NLRP3 protein having one or both of a R920Q amino acid substitution (each numbered according to the mature NLRP3 protein sequence of SEQ ID NO: 35) in a cell from the subject. In some embodiments of any of the methods described herein, the subject identified as having a cell that has an elevated level of NLRP3 inflammasome activity has been determined to have a cell having a mutation in an NLRP3 gene that results in the expression of an NLRP3 protein having a R920Q amino acid substitution (each numbered according to the mature NLRP3 protein sequence of SEQ ID NO: 35). In some embodiments of these methods, the subject has or is suspected of having deafness with or without inflammation.

In some embodiments of any of the methods described herein, the identifying a subject having a cell that has an elevated level of NLRP3 inflammasome activity comprises detecting a mutation in an NLRP3 gene that results in the expression of an NLRP3 protein having a D21H amino acid substitution (numbered according to the mature NLRP3 protein sequence of SEQ ID NO: 35) in a cell from the subject. In some embodiments of any of the methods described herein, the subject identified as having a cell that has an elevated level of NLRP3 inflammasome activity has been determined to have a cell having an NLRP3 gene that results in the expression of an NLRP3 protein having a D21H amino acid substitution (numbered according to the mature NLRP3 protein sequence of SEQ ID NO: 35). In some embodiments of these methods, the subject has or is suspected of having keratoendotheliitis fugax hereditaria.

In some embodiments of any of the methods described herein, the subject has or is suspected of having an inappropriate host response to infectious diseases where active infection exists at any body site. In some embodiments of these methods, the inappropriate host response to infectious disease where active infection exists at any body site is selected from the group of: septic shock, disseminated intravascular coagulation, and adult respiratory distress syndrome.

In some embodiments of any of the methods described herein, the subject has or is suspected of having acute or chronic inflammation due to antigen, antibody, and/or complement deposition. In some embodiments of any of the methods described herein, the subject has or is suspected of having an inflammatory disease or condition (e.g., an inflammatory disease or conditions selected from the group of arthritis, cholangitis, colitis, encephalitis, endocarditis, glomerulonephritis, hepatitis, myocarditis, pancreatitis, pericarditis, reperfusion injury, vasculitis, osteoarthritis, COPD, periodontal disease, uveitis, cutaneous T-cell lymphoma, and mucositis such as oral mucositis, esophageal mucositis, and intestinal mucositis).

In some embodiments of any of the methods described herein, the subject has or is suspected of having acute and delayed hypersensitivity, graft rejection, or graft-versus-host disease (GVHD). In some embodiments of any of the methods described herein, the subject has or is suspected of having an autoimmune disease selected from the group consisting of: Type 1 diabetes mellitus, rheumatoid arthritis, systemic lupus erythematosus, autoimmune thyroiditis, Addison's disease, pernicious anemia, multiple sclerosis, an inflammatory bowel disease (IBD) (e.g., an IBD selected from the group of Crohn's disease, ulcerative colitis, autoimmune colitis, iatrogenic autoimmune colitis, ulcerative colitis, colitis induced by one or more chemotherapeutic agents, colitis induced by treatment with adoptive cell therapy, colitis associated with one or more alloimmune diseases such as GVHD, radiation enteritis, collagenous colitis, lymphocytic colitis, microscopic colitis, and radiation enteritis, celiac disease, and inflammatory bowel syndrome), scleroderma, and psoriasis.

In some embodiments of any of the methods described herein, the subject has or is suspected of having a metabolic disorder (e.g., a metabolic disorder selected from the group of type 2 diabetes, atherosclerosis, obesity, gout, and pseudogout). In some embodiments of any of the methods described herein, the subject has or is suspected of having a disease of the central nervous system (e.g., a disease of the central nervous system selected from the group of Alzheimer's disease, multiple sclerosis, amyotrophic lateral sclerosis, and Parkinson's disease).

In some embodiments of any of the methods described herein, the subject has or is suspected of having a lung disease (e.g., asthma, COPD, pulmonary idiopathic fibrosis, or cystic fibrosis). In some embodiments of any of the methods described herein, the subject has or is suspected of having a liver disease (e.g., NASH syndrome, viral hepatitis, or cirrhosis). In some embodiments of any of the methods described herein, wherein the subject has or is suspected of having a pancreatic disease (e.g., acute or chronic pancreatitis). In some embodiments of any of the methods described herein, wherein the subject has or is suspected of having kidney disease (e.g., as acute or chronic kidney injury). In some embodiments of any of the methods described herein, the subject has or is suspected of having an intestinal disease (e.g., Crohn's disease or ulcerative colitis).

In some embodiments of any of the methods described herein, the subject has or is suspected of having a skin disease (e.g., psoriasis). In some embodiments of any of the methods described herein, the subject has or is suspected of having a musculoskeletal disease (e.g., scleroderma). In some embodiments of any of the methods described herein, the subject has or is suspected of having a vessel disorder (e.g., giant cell arteritis). In some embodiments of any of the methods described herein, the subject has a bone disorder (e.g., osteoarthritis, osteoporosis, or osteopetrosis disorders).

In some embodiments of any of the methods described herein, the subject has or is suspected of having an eye disease (e.g., glaucoma or macular degeneration, e.g., age-related macular degeneration). In some embodiments of any of the methods described herein, the subject has or is suspected of having a disease caused by viral infection (e.g., HIV or AIDS). In some embodiments of any of the methods described herein, the subject has or is suspected of having a cancer (e.g., non-small cell lung cancer, acute lymphoblastic leukemia (ALL) (e.g., ALL in patients resistant to glucocorticoid treatment), multiple myeloma, promyelocytic leukemia, gastric cancer, or lung cancer metastasis). In some embodiments of any of the methods described herein, the subject has or is suspected of having a cardiovascular disease (e.g., myocardial infarction, stroke, or heart failure).

In some embodiments of any of the methods described herein, the subject has or is suspected of having: hereditary periodic fever, familial cold autoinflammatory syndrome (FCAS), Muckle-Wells syndrome, myelodysplastic syndrome (MDS), Langerhan's cell histiocytosis (LCH), neonatal onset multisystem inflammatory disease, Cinca syndrome, deafness with inflammation, deafness without inflammation, keratoendotheliitis fugax hereditaria, silicosis, asbestosis, or chronic neurologic cutaneous and articular syndrome. In some embodiments of any of the methods described herein, the subject has been exposed to, or is suspected of having been exposed to, a toxic agent selected from the group of: exogenous microbial stimuli, lipopolysaccharide (LPS), lipooligosaccharide, muramyl dipeptide (MDP), nigericin, maitotoxin, asbestos, and silica.

Provided herein are methods of treating a subject (e.g., any of the exemplary subjects described herein) in need thereof that include: (a) identifying a subject having resistance to an anti-TNFα agent (e.g., any of the exemplary types of resistance to an anti-TNFα agent described herein or known in the art, or any of the types of anti-TNFα agents described herein or known in the art); and (b) administering a treatment comprising a therapeutically effective amount of an NLRP3 antagonist (e.g., any of the exemplary NLRP3 antagonists described herein) or a pharmaceutically acceptable salt, solvate, or co-crystal thereof to the identified subject. In some examples of these methods, step (b) can further include identifying the subject as also having an elevated level of NLRP3 inflammasome activity and/or expression in a cell obtained from the subject, as compared to a reference level (e.g., any of the exemplary reference levels described herein). In some embodiments of these methods, the treatment can further include a therapeutically effective amount of an anti-TNFα agent (e.g., any of the exemplary anti-TNFα agents described herein), in addition to the NLRP3 antagonist.

Also provided herein are methods of treating a subject (e.g., any of the exemplary subjects described herein) in need thereof, that include: administering a treatment comprising a therapeutically effective amount of an NLRP3 antagonist (e.g., any of the exemplary NLRP3 antagonists described herein) or a pharmaceutically acceptable salt, solvate, or co-crystal thereof to a subject identified as having resistance to an anti-TNFα agent (e.g., any of the exemplary types of resistance to an anti-TNFα agent described herein or known in the art, or any of the anti-TNFα agents described herein or known in the art). In some embodiments of these methods, the identified subject also has an elevated level of level of NLRP3 inflammasome activity and/or expression in a cell obtained from the subject, as compared to a reference level.expression in a cell obtained from the subject, as compared to a reference level (e.g., any of the exemplary reference levels described herein or known in the art). In some embodiments of these methods, the treatment further includes a therapeutically effective amount of an anti-TNFα agent (e.g., any of the exemplary anti-TNFα agents described herein or known in the art), in addition to the NLRP3 antagonist.

Also provided herein are methods of treating a subject (e.g., any of the exemplary subjects described herein) in need thereof, that include: (a) administering one or more doses of an anti-TNFα agent (e.g., any of the exemplary anti-TNFα agents described herein) to the subject; (b) detecting an elevated level (e.g., an increase of 1% to 1000%, or any of the subranges of this range described herein) of NLRP3 inflammasome activity and/or expression in a cell obtained from the subject after step (a) as compared to a reference level (e.g., any of the exemplary reference levels of NLRP3 inflammasome activity and/or expression described herein or known in the art); and (c) administering a treatment comprising a therapeutically effective amount of an NLRP3 antagonist (e.g., any of the exemplary NLRP3 antagonists described herein or known in the art) or a pharmaceutically acceptable salt, solvate, or co-crystal thereof to a subject determined to have an elevated level (e.g., an increase of 1% to 1000%, or any of the subranges of this range described herein) of NLRP3 inflammasome activity and/or expression as compared to the reference level in step (b). In some embodiments of any of these methods, the treatment can further include a therapeutically effective amount of an anti-TNFα agent (e.g., any of the exemplary anti-TNFα agents described herein or known in the art), in addition to the NLRP3 antagonist. In some embodiments of these methods, the treatment can further include a therapeutically effective amount of the previously administered anti-TNFα agent, in addition to the NLRP3 antagonist.

Also provided herein are methods of treating a subject (e.g., any of the exemplary subjects described herein) in need thereof, that include: (a) detecting an elevated level (e.g., an increase of 1% to 1000%, or any of the subranges of this range described herein) of NLRP3 inflammasome activity and/or expression as compared to a reference level (e.g., any of the exemplary reference levels of NLRP3 inflammasome activity described herein or known in the art) in a cell obtained from a subject previously administered one or more doses of an anti-TNFα agent; and (b) administering a treatment comprising a therapeutically effective amount of an NLRP3 antagonist (e.g., any of the exemplary NLRP3 antagonsits described herein or known in the art) or a pharmaceutically acceptable salt, solvate, or co-crystal thereof to a subject determined to have an elevated level (e.g., an increase of 1% to 1000%, or any of the subranges of this range described herein) of NLRP3 inflammasome activity and/or expression as compared to the reference level in step (a). In some embodiments of any of these methods, the treatment can further include a therapeutically effective amount of an anti-TNFα agent (e.g., any of the exemplary anti-TNFα agents described herein or known in the art), in addition to the NLRP3 antagonist. In some embodiments of these methods, the treatment can further include a therapeutically effective amount of the previously administered anti-TNFα agent, in addition to the NLRP3 antagonist.

Also provided herein are methods of treating a subject (e.g., any of the exemplary subjects described herein) in need thereof, that include: administering a treatment comprising a therapeutically effective amount of an NLRP3 antagonist (e.g., any of the exemplary NLRP3 antagonists described herein or known in the art) or a pharmaceutically acceptable salt, solvate, or co-crystal thereof to a subject determined to have an elevated level (e.g., an increase of 1% to 1000%, or any of the subranges of this range described herein) of NLRP3 inflammasome activity and/or expression as compared to a reference level (e.g., any of the exemplary reference levels of NLRP3 inflammasome activity described herein or known in the art) in a cell obtained from the subject after previous administration with one or more doses of an anti-TNFα agent (e.g., any of the exemplary anti-TNFα agents described herein or known in the art). In some embodiments of these methods, the treatment can further include a therapeutically effective amount of an anti-TNFα agent (e.g., any of the exemplary anti-TNFα agents described herein or known in the art), in addition to the NLRP3 antagonist. In some embodiments of these methods, the treatment can further include a therapeutically effective amount of the previously administered anti-TNFα agent, in addition to the NLRP3 antagonist.

Provided herein are methods of treating a subject (e.g., any of the exemplary subjects described herein) in need thereof, that include: (a) administering one or more doses of an anti-TNFα agent (e.g., any of the exemplary anti-TNFα agents described herein or known in the art) to the subject; (b) after step (a), detecting resistance to the anti-TNFα agent in the subject (e.g., any of the exemplary types of resistance to an anti-TNFα agent described herein or known in the art); and (c) administering a treatment comprising a therapeutically effective amount of an NLRP3 antagonist (e.g., any of the exemplary types of NLRP3 antagonists described herein or known in the art) or a pharmaceutically acceptable salt, solvate, or co-crystal thereof to a subject determined to have resistance to the anti-TNFα agent in step (b). In some embodiments of these methods, the treatment further includes a therapeutically effective amount of an anti-TNFα agent (e.g., any of the exemplary anti-TNFα agents described herein or known in the art), in addition to the NLRP3 antagonist. In some embodiments of these methods, the treatment can further include a therapeutically effective amount of the anti-TNFα agent administered in step (a), in addition to the NLRP3 antagonist.

Also provided herein are methods of treating a subject (e.g., any of the exemplary subjects described herein) in need thereof, that include: (a) detecting resistance to an anti-TNFα agent (e.g., any of the exemplary types of resistance to an anti-TNFα agent described herein or known in the art) in a subject previously administered one or more doses of the anti-TNFα agent (e.g., any of the exemplary types of an anti-TNFα agent described herein or known in the art); and (b) administering a treatment comprising a therapeutically effective amount of an NLRP3 antagonist (e.g., any of the exemplary NLRP3 antagonists described herein or known in the art) or a pharmaceutically acceptable salt, solvate, or co-crystal thereof to a subject determined to have resistance to the anti-TNFα agent in step (a). In some embodiments of these methods, the treatment further includes a therapeutically effective amount of an anti-TNFα agent (e.g., any of the exemplary anti-TNFα agents described herein or known in the art), in addition to the NLRP3 antagonist. In some embodiments of these methods, the treatment can further include a therapeutically effective amount of the previously administered anti-TNFα agent, in addition to the NLRP3 antagonist.

Also provided herein are methods of treating a subject (e.g., any of the exemplary subjects described herein) in need thereof, that include: comprising administering a treatment comprising a therapeutically effective amount of an NLRP3 antagonist (e.g., any of the exemplary NLRP3 antagonists described herein or known in the art) or a pharmaceutically acceptable salt, solvate, or co-crystal thereof to a subject previously administered one or more doses of an anti-TNFα agent (e.g., any of the exemplary anti-TNFα agents described herein or known in the art) and determined to have resistance to the anti-TNFα agent (e.g., any of the exemplary types of resistance to an anti-TNFα agent described herein or known in the art). In some embodiments of these methods, the treatment further includes a therapeutically effective amount of an anti-TNFα agent (e.g., any of the exemplary anti-TNFα agents described herein or known in the art), in addition to the NLRP3 antagonist. In some embodiments of these methods, the treatment can further include a therapeutically effective amount of the previously administered anti-TNFα agent, in addition to the NLRP3 antagonist.

Also provided herein are methods of treating a subject (e.g., any of the exemplary subjects described herein) in need thereof, that include: (a) identifying a subject having an elevated level (e.g., an increase of 1% to 1000%, or any of the subranges of this range described herein) of NLRP3 inflammasome activity and/or expression in a cell obtained from the subject, as compared to a reference level (e.g., any of the exemplary reference levels of NLRP3 inflammasome activity described herein or known in the art); and (b) administering a treatment comprising (i) a therapeutically effective amount of an NLRP3 antagonist (e.g., any of the exemplary NLRP3 antagonists described herein or known in the art) or a pharmaceutically acceptable salt, solvate, or co-crystal thereof, and (ii) a therapeutically effective amount of an anti-TNFα agent (e.g., any of the exemplary anti-TNFα agents described herein or known in the art) to the identified subject. In some embodiments of these methods, the subject has not previously been administered an anti-TNFα agent.

Also provided herein are methods of treating a subject (e.g., any of the exemplary subjects described herein) in need thereof, that include: administering a treatment comprising (i) a therapeutically effective amount of an NLRP3 antagonist (e.g., any of the exemplary NLRP3 antagonists described herein or known in the art) or a pharmaceutically acceptable salt, solvate, or co-crystal thereof, and (ii) a therapeutically effective amount of an anti-TNFα agent (e.g., any of the exemplary anti-TNFα agents described herein or known in the art), to a subject identified as having an elevated level (e.g., an increase of 1% to 1000%, or any of the subranges of this range described herein) of NLRP3 inflammasome activity and/or expression in a cell obtained from the subject, as compared to a reference level (e.g., any of the exemplary reference levels of NLRP3 inflammasome activity described herein or known in the art). In some embodiments of these methods, the subject has not previously been administered an anti-TNFα agent.

In some embodiments of any of the methods described herein, the NLRP3 antagonist is a compound of any one of Formulas I-X, or a pharmaceutically acceptable salt, solvate, or co-crystal thereof, and is administered to a subject in need thereof, in combination with an anti-TNFα agent; preferably in combination with Infliximab, Etanercept, Certolizumab pegol, Golimumab or Adalimumab; more preferably in combination with Adalimumab; more preferably in combination with Adalimumab.

In some embodiments of any of the methods described herein, the NLRP3 inflammasome activity is secretion of IL-18 (processed IL-18). In some embodiments of any of the methods described herein, the NLRP3 inflammasome activity is secretion of IL-1β (processed IL-1∂). In some embodiments of any of the methods described herein, the NLRP3 inflammasome activity is caspase-1 activity. In some embodiments of any of the methods described herein, the NLRP3 inflammasome activity is the level of lipocalin-2. In some embodiments of any of the methods described herein, the NLRP3 inflammasome activity is the level of S100A8. In some embodiments of any of the methods described herein, the NLRP3 inflammasome activity is the level of S100A9.

In some embodiments of any of the methods described herein, the identifying of the subject as having a cell that has an elevated level of NLRP3 inflammasome expression includes detecting the level of one or more of NLRP3 protein, ASC protein, procaspase-1 protein, and caspase-1 protein. In some embodiments of any of the methods described herein, the identifying the subject as also having a cell that has an elevated level of NLRP3 inflammasome expression includes detecting the level of one or more of NLRP3 mRNA, ASC mRNA, and procaspase-1 mRNA.

In some embodiments of any of the methods described herein, the identifying of the subject as having a cell that has an elevated level of NLRP3 inflammasome activity includes detecting the level of one or more of CRP protein, SAA protein, HP protein, ceruloplasmin protein, IL-6 protein (e.g., mature or pro-IL-6 protein), calprotectin (S100A8) protein, IL-8 protein (e.g., mature or pro-IL-8 protein), leukotriene B4 protein, and myeloperoxidase protein. In some embodiments of any of the methods described herein, the identifying of the subject as having a cell that has an elevated level of one or more of CRP mRNA, SAA mRNA, HP mRNA, ceruloplasmin mRNA, pro-IL-6 mRNA, calprotectin (S100A8) mRNA, pro-IL-8 mRNA, leukotriene B4 mRNA, and myeloperoxidase mRNA.

In some embodiments of any of the methods described herein, where the subject is administered a therapeutically effective amount of an NLRP3 antagonist and a therapeutically effective amount of an anti-TNFα agent, the NLRP3 antagonist and the anti-TNFα agent can be administered at substantially the same time (e.g., in different dosage forms or formulated together into a single dosage form). In some embodiments of any of the methods described herein, wherein the subject is administered a therapeutically effective amount of an NLRP3 antagonist and a therapeutically effective amount of an anti-TNFα agent, the subject can be first administered the NLRP3 antagonist and then be administered the anti-TNFα agent. In some embodiments of any of the methods described herein, wherein the subject is administered a therapeutically effective amount of an NLRP3 antagonist and a therapeutically effective amount of an anti-TNFα agent, the subject can be first administered the anti-TNFα agent and then be administered the NLRP3 antagonist.

In some embodiments of any of the methods described herein, the subject has been diagnosed or identified as having an inflammatory or an autoimmune disorder (e.g., any of the exemplary inflammatory or autoimmune disorders described herein or known in the art). For example, in some embodiments of any of the methods described herein, the subject can be diagnosed or identified as having Crohn's disease, ulcerative colitis, or inflammatory bowel disease.

In some embodiments of any of the methods described herein, the anti-TNFα agent is an antibody or an antigen-binding antibody fragment (e.g., adalimumab, certolizumab, etanercept, golimumab, infliximab, CDP571, or certolizumab pegol), or a soluble TNFα receptor. In some embodiments of any of the methods described herein, the anti-TNFα agent is a small molecule inhibitor of a signaling component downstream of a TNFα receptor. In some embodiments of any of the methods described herein, the anti-TNFα agent is an inhibitory nucleic acid. In some embodiments of any of the methods described herein, the anti-TNFα agent is a fusion protein. In some embodiments of any of the methods described herein, the NLRP3 antagonist is an inhibitory nucleic acid (e.g., a short interfering RNA, an antisense nucleic acid, or a ribozyme). In some embodiments of any of the methods described herein, the NLRP3 antagonist is a compound of any one of Formulas I-X, or a pharmaceutically acceptable salt, solvate, or co-crystal thereof. In some embodiments of any of the methods described herein, the NLRP3 antagonist is a compound shown in any one of Tables 1-18, or a pharmaceutically acceptable salt, solvate, or co-crystal thereof.

In some embodiments of any of the methods of treatment described herein, the method can result in a decreased risk (e.g., a 1% to a 99% decrease, e.g., a 1% to 95% decrease, a 1% to 90% decrease, a 1% to 85% decrease, a 1% to 80% decrease, a 1% to 75% decrease, a 1% to 70% decrease, a 1% to 65% decrease, a 1% to 60% decrease, a 1% to 55% decrease, a 1% to 50% decrease, a 1% to 45% decrease, a 1% to 40% decrease, a 1% to 35% decrease, a 1% to 30% decrease, a 1% to 25% decrease, a 1% to 20% decrease, a 1% to 15% decrease, a 1% to 10% decrease, a 1% to 5% decrease, a 5% to 99% decrease, a 5% to 95% decrease, a 5% to 90% decrease, a 5% to 85% decrease, a 5% to 80% decrease, a 5% to 75% decrease, a 5% to 70% decrease, a 5% to 65% decrease, a 5% to 60% decrease, a 5% to 55% decrease, a 5% to 50% decrease, a 5% to 45% decrease, a 5% to 40% decrease, a 5% to 35% decrease, a 5% to 30% decrease, a 5% to 25% decrease, a 5% decrease to 20% decrease, a 5% to 15% decrease, a 5% to 10% decrease, a 10% to 99% decrease, a 10% to 95% decrease, a 10% to 90% decrease, a 10% to 85% decrease, a 10% to 80% decrease, a 10% to 75% decrease, a 10% to 70% decrease, a 10% to 65% decrease, a 10% to 60% decrease, a 10% to 55% decrease, a 10% to 50% decrease, a 10% to 45% decrease, a 10% to 40% decrease, a 10% to 35% decrease, a 10% to 30% decrease, a 10% to 25% decrease, a 10% decrease to 20% decrease, a 10% to 15% decrease, a 15% to 99% decrease, a 15% to 95% decrease, a 15% to 90% decrease, a 15% to 85% decrease, a 15% to 80% decrease, a 15% to 75% decrease, a 15% to 70% decrease, a 15% to 65% decrease, a 15% to 60% decrease, a 15% to 55% decrease, a 15% to 50% decrease, a 15% to 45% decrease, a 15% to 40% decrease, a 15% to 35% decrease, a 15% to 30% decrease, a 15% to 25% decrease, a 15% decrease to 20% decrease, a 20% to 99% decrease, a 20% to 95% decrease, a 20% to 90% decrease, a 20% to 85% decrease, a 20% to 80% decrease, a 20% to 75% decrease, a 20% to 70% decrease, a 20% to 65% decrease, a 20% to 60% decrease, a 20% to 55% decrease, a 20% to 50% decrease, a 20% to 45% decrease, a 20% to 40% decrease, a 20% to 35% decrease, a 20% to 30% decrease, a 20% to 25% decrease, a 25% to 99% decrease, a 25% to 95% decrease, a 25% to 90% decrease, a 25% to 85% decrease, a 25% to 80% decrease, a 25% to 75% decrease, a 25% to 70% decrease, a 25% to 65% decrease, a 25% to 60% decrease, a 25% to 55% decrease, a 25% to 50% decrease, a 25% to 45% decrease, a 25% to 40% decrease, a 25% to 35% decrease, a 25% to 30% decrease, a 30% to 99% decrease, a 30% to 95% decrease, a 30% to 90% decrease, a 30% to 85% decrease, a 30% to 80% decrease, a 30% to 75% decrease, a 30% to 70% decrease, a 30% to 65% decrease, a 30% to 60% decrease, a 30% to 55% decrease, a 30% to 50% decrease, a 30% to 45% decrease, a 30% to 40% decrease, a 30% to 35% decrease, a 35% to 99% decrease, a 35% to 95% decrease, a 35% to 90% decrease, a 35% to 85% decrease, a 35% to 80% decrease, a 35% to 75% decrease, a 35% to 70% decrease, a 35% to 65% decrease, a 35% to 60% decrease, a 35% to 55% decrease, a 35% to 50% decrease, a 35% to 45% decrease, a 35% to 40% decrease, a 40% to 99% decrease, a 40% to 95% decrease, a 40% to 90% decrease, a 40% to 85% decrease, a 40% to 80% decrease, a 40% to 75% decrease, a 40% to 70% decrease, a 40% to 65% decrease, a 40% to 60% decrease, a 40% to 55% decrease, a 40% to 50% decrease, a 40% to 45% decrease, a 45% to 99% decrease, a 45% to 95% decrease, a 45% to 90% decrease, a 45% to 85% decrease, a 45% to 80% decrease, a 45% to 75% decrease, a 45% to 70% decrease, a 45% to 65% decrease, a 45% to 60% decrease, a 45% to 55% decrease, a 45% to 50% decrease, a 50% to 99% decrease, a 50% to 95% decrease, a 50% to 90% decrease, a 50% to 85% decrease, a 50% to 80% decrease, a 50% to 75% decrease, a 50% to 70% decrease, a 50% to 65% decrease, a 50% to 60% decrease, a 50% to 55% decrease, a 55% to 99% decrease, a 55% to 95% decrease, a 55% to 90% decrease, a 55% to 85% decrease, a 55% to 80% decrease, a 55% to 75% decrease, a 55% to 70% decrease, a 55% to 65% decrease, a 55% to 60% decrease, a 60% to 99% decrease, a 60% to 95% decrease, a 60% to 90% decrease, a 60% to 85% decrease, a 60% to 80% decrease, a 60% to 75% decrease, a 60% to 70% decrease, a 60% to 65% decrease, a 65% to 99% decrease, a 65% to 95% decrease, a 65% to 90% decrease, a 65% to 85% decrease, a 65% to 80% decrease, a 65% to 75% decrease, a 65% to 70% decrease, a 70% to 99% decrease, a 70% to 95% decrease, a 70% to 90% decrease, a 70% to 85% decrease, a 70% to 80% decrease, a 70% to 75% decrease, a 75% to 99% decrease, a 75% to 95% decrease, a 75% to 90% decrease, a 75% to 85% decrease, a 75% to 80% decrease, a 80% to 99% decrease, a 80% to 95% decrease, a 80% to 90% decrease, a 80% to 85% decrease, a 85% to 99% decrease, a 85% to 95% decrease, a 85% to 90% decrease, a 90% to 99% decrease, a 90% to 95% decrease, or a 95% to 99% decrease) of developing a comorbidity in the subject (e.g., as compared to the risk of developing a comorbidity in a subject having a similar elevated level of NLRP3 inflammasome activity and/or expression in a cell and/or a similar level of anti-TNFα resistance, but administered a different treatment or a placebo).

In some embodiments of any of the methods described herein, where the subject has inflammatory bowel disease, such as ulcerative colitis or Crohn's, the methods can result in a decrease (e.g., a 1% to 99% decrease, or any of the subranges of this range described herein) in the disease activity index (DAI) for Crohn's disease or ulcerative colitis in the subject (e.g., as compared to the DAI in the same subject prior to treatment).

In some embodiments of any of the methods described herein, wherein the subject has inflammatory bowel syndrome, such as Crohn's disease or ulcerative colitis, the methods can result in an improvement in stool consistency in the subject (e.g., as compared to the stool consistency in the subject prior to treatment).

In some embodiments of any of the methods described herein, the methods can result in a decrease (e.g., a 1% to 99% decrease, or any of the subranges of this range) in the severity, duration, or number of symptoms of an inflammatory disease or autoimmune disease (e.g., any of the inflammatory diseases or autoimmune diseases described herein or known in the art, e.g., inflammatory bowel disease, such as ulcerative colitis, Crohn's disease) in the subject. Non-limiting examples of symptoms of ulcerative colitis and Crohn's disease in a subject include: abdominal pain, diarrhea, blood stool, fever, unintended weight loss, fatigue, abdominal cramping, and mouth sores.

Additional exemplary aspects that can be used or incorporated in these methods are described herein.

Methods of Predicting a Subject's Responsiveness to an Anti-TNFα Agent

Also provided herein are methods of predicting a subject's responsiveness (e.g., any of the exemplary subjects described herein) to an anti-TNFα agent (e.g., any of the exemplary anti-TNFα agents described herein or known in the art), that include: (a) determining that a subject has an elevated level (e.g., an increase of 1% to 1000%, or any of the subranges of this range described herein) of NLRP3 inflammasome activity and/or expression in a cell obtained from the subject, as compared to a reference level (e.g., any of the exemplary reference levels of NLRP3 inflammasome activity described herein or known in the art); and (b) identifying that the subject determined to have an elevated level of NLRP3 inflammasome activity and/or expression in step (a) has an increased likelihood of being resistant to treatment with an anti-TNFα agent.

Also provided herein are methods of predicting a subject's responsiveness (e.g., any of the exemplary subjects described herein) to an anti-TNFα agent (e.g., any of the exemplary anti-TNFα agents described herein or known in the art), that include: identifying a subject determined to have an elevated level (e.g., an increase of 1% to 1000%, or any of the subranges of this range described herein) of NLRP3 inflammasome activity and/or expression in a cell obtained from the subject, as having an increased likelihood of being resistant to treatment with an anti-TNFα agent.

In some embodiments of any of the methods described herein, the NLRP3 inflammasome activity is secretion of IL-18 (processed IL-18). In some embodiments of any of the methods described herein, the NLRP3 inflammasome activity is secretion of IL-1β (processed IL-1∂). In some embodiments of any of the methods described herein, the NLRP3 inflammasome activity is caspase-1 activity. In some embodiments of any of the methods described herein, the NLRP3 inflammasome activity is the level of lipocalin-2. In some embodiments of any of the methods described herein, the NLRP3 inflammasome activity is the level of S100A8. In some embodiments of any of the methods described herein, the NLRP3 inflammasome activity is the level of S100A9.

In some embodiments of any of the methods described herein, the determining that a subject has an elevated level of NLRP3 inflammasome activity and/or expression includes detecting the level of one or more (e.g., 1, 2, 3, or 4) of NLRP3 protein, ASC protein, procaspase-1 protein, and caspase-1 protein. In some embodiments of any of the methods described herein, the determining that a subject has an elevated level of NLRP3 inflammasome expression includes detecting the level of one or more (e.g., 1, 2, 3, or 4) of NLRP3 mRNA, ASC mRNA, and procaspase-1 mRNA.

In some embodiments of any of the methods described herein, the identifying of the subject as having a cell that has an elevated level of NLRP3 inflammasome activity includes detecting the level of one or more of CRP protein, SAA protein, HP protein, ceruloplasmin protein, IL-6 protein (e.g., mature or pro-IL-6 protein), calprotectin (S100A8) protein, IL-8 protein (e.g., mature or pro-IL-8 protein), leukotriene B4 protein, and myeloperoxidase protein. In some embodiments of any of the methods described herein, the identifying of the subject as having a cell that has an elevated level of one or more of CRP mRNA, SAA mRNA, HP mRNA, ceruloplasmin mRNA, pro-IL-6 mRNA, calprotectin (S100A8) mRNA, pro-IL-8 mRNA, leukotriene B4 mRNA, and myeloperoxidase mRNA.

In some embodiments of any of the methods described herein, the subject determined to have an elevated level of NLRP3 inflammasome expression has been determined to have a cell having an elevated level of one or more of NLRP3 protein, ASC protein, procaspase-1 protein, and capsase-1 protein. In some embodiments of any of the methods described herein, the subject determined to have an elevated level of NLRP3 inflammasome expression has been determined to have a cell having an elevated level of one or more of NLRP3 mRNA, ASC mRNA, and procaspase-1 mRNA.

In some embodiments of any of the methods described herein, the subject has not previously been administered a dose of an anti-TNFα antagonist.

Some embodiments of any of the methods described herein can further include: administering to the subject identified as having an increased likelihood of being resistant to treatment with an anti-TNFα agent, a treatment including (i) a therapeutically effective amount of an anti-TNFα agent (e.g., any of the exemplary anti-TNFα agent described herein) and (ii) a therapeutically effective amount of an NLRP3 antagonist (e.g., any of the exemplary NLRP3 antagonsits described herein or known in the art).

Some embodiments of any of the methods described herein can further include: recording the identification of the subject as having an increased likelihood of being resistant to treatment with an anti-TNFα agent in the subject's clinical record (e.g., a computer readable medium). Some embodiments of any of the methods described herein can further include recording in the clinical record for the subject identified as having an increased likelihood of being resistant to treatment with an anti-TNFα agent, that the subject should be administered an NLRP3 antagonist (e.g., alone or in combination with an anti-TNFα agent). Additional exemplary aspects that can be used or incorporated in these methods are described herein.

In some embodiments of any of the methods described herein, the NLRP3 antagonist is an inhibitory nucleic acid (e.g., a short interfering RNA, an antisense nucleic acid, or a ribozyme). In some embodiments of any of the methods described herein, the NLRP3 antagonist is a compound of any one of Formulas I-X, or a pharmaceutically acceptable salt, solvate, or co-crystal thereof. In some embodiments of any of the methods described herein, the NLRP3 antagonist is a compound shown in any one of Tables 1-18, or a pharmaceutically acceptable salt, solvate, or co-crystal thereof.

In some embodiments of any of the methods described herein, the NLRP3 antagonist is a compound of any one of Formulas I-X, or a pharmaceutically acceptable salt, solvate, or co-crystal thereof, and is administered to a subject in need thereof, in combination with an anti-TNFα agent; preferably in combination with Infliximab, Etanercept, Certolizumab pegol, Golimumab or Adalimumab; more preferably in combination with Adalimumab; more preferably in combination with Adalimumab.

Some embodiments of any of the methods described herein can further include recording the selected treatment in the subject's clinical record (e.g., a computer readable medium). Some embodiments of any of the methods described herein can further include administering one or more doses (e.g., at least two, at least four, at least six, at least eight, at least ten doses) of the selected treatment to the identified subject.

Additional exemplary aspects that can be used or incorporated in these methods are described herein.

Methods of Selecting a Subject for Treatment

Also provided herein are methods of selecting a treatment for a subject (e.g., any of the exemplary subjects described herein) in need thereof, that include: (a) identifying a subject having resistance to an anti-TNFα agent (e.g., any of the exemplary resistances to an anti-TNFα agent described herein or known in the art); and (b) selecting for the identified subject a treatment comprising a therapeutically effective amount of an NLRP3 antagonist (e.g., any of the exemplary NLRP3 antagonists described herein or known in the art) or a pharmaceutically acceptable salt, solvate, or co-crystal thereof. In some embodiments of these methods, step (b) can further include identifying the subject as also having an elevated level of NLRP3 inflammasome activity and/or expression in a cell obtained from the subject, as compared to a reference level. In some embodiments of these methods, the treatment further includes a therapeutically effective amount of an anti-TNFα agent (e.g., a previously administered anti-TNFα agent), in addition to the NLRP3 antagonist.

Also provided herein are methods of selecting a treatment for a subject (e.g., any of the exemplary subjects described herein) in need thereof, that include: selecting a treatment comprising a therapeutically effective amount of an NLRP3 antagonist (e.g., any of the exemplary NLRP3 antagonists described herein or known in the art) or a pharmaceutically acceptable salt, solvate, or co-crystal thereof for a subject identified as having resistance to an anti-TNFα agent (e.g., any of the exemplary resistances to an anti-TNFα agent described herein or known in the art). In some embodiments of these methods, the identified subject also has an elevated level of level of NLRP3 inflammasome activity and/or expression in a cell obtained from the subject, as compared to a reference level. In some embodiments of these methods, the treatment further includes a therapeutically effective amount of an anti-TNFα agent (e.g., a previously administered anti-TNFα agent), in addition to the NLRP3 antagonist.

Provided herein are methods of selecting a treatment for a subject (e.g., any of the exemplary subjects described herein) in need thereof, that include: (a) identifying a subject having an elevated level (e.g., an increase of 1% to 1000%, or any of the subranges of this range described herein) of NLRP3 inflammasome activity and/or expression in a cell obtained from the subject, as compared to a reference level (e.g., any of the exemplary reference levels of NLRP3 inflammasome activity described herein or known in the art); and (b) selecting for the identified subject a treatment comprising (i) a therapeutically effective amount of an NLRP3 antagonist (e.g., any of the exemplary NLRP3 antagonists described herein or known in the art) or a pharmaceutically acceptable salt, solvate, or co-crystal thereof, and (ii) a therapeutically effective amount of an anti-TNFα agent (e.g., any of the exemplary anti-TNFα agents described herein or known in the art). In some embodiments of these methods, the subject has not previously been administered an anti-TNFα agent.

Also provided herein are methods of selecting a treatment for a subject (e.g., any of the exemplary subjects described herein) in need thereof, that include: selecting a treatment comprising (i) a therapeutically effective amount of an NLRP3 antagonist (e.g., any of the exemplary NLRP3 antagonists described herein or known in the art) or a pharmaceutically acceptable salt, solvate, or co-crystal thereof, and (ii) a therapeutically effective amount of an anti-TNFα agent (e.g., any of the exemplary anti-TNFα agents described herein or known in the art), for a subject identified as having an elevated level (e.g., an increase of 1% to 1000%, or any of the subranges of this range described herein) of NLRP3 inflammasome activity and/or expression in a cell obtained from the subject, as compared to a reference level (e.g., any of the exemplary reference levels of NLRP3 inflammasome activity described herein or known in the art). In some embodiments of these methods, the subject has not previously been administered an anti-TNFα agent.

In some embodiments of any of the methods described herein, the NLRP3 inflammasome activity is secretion of IL-18 (processed IL-18). In some embodiments of any of the methods described herein, the NLRP3 inflammasome activity is secretion of IL-1β (processed IL-1∂). In some embodiments of any of the methods described herein, the NLRP3 inflammasome activity is caspase-1 activity. In some embodiments of any of the methods described herein, the NLRP3 inflammasome activity is the level of lipocalin-2. In some embodiments of any of the methods described herein, the NLRP3 inflammasome activity is the level of S100A8. In some embodiments of any of the methods described herein, the NLRP3 inflammasome activity is the level of S100A9.

In some embodiments of any of the methods described herein, the identifying of the subject as having a cell that has an elevated level of NLRP3 inflammasome activity includes detecting the level of one or more of CRP protein, SAA protein, HP protein, ceruloplasmin protein, IL-6 protein (e.g., mature or pro-IL-6 protein), calprotectin (S100A8) protein, IL-8 protein (e.g., mature or pro-IL-8 protein), leukotriene B4 protein, and myeloperoxidase protein. In some embodiments of any of the methods described herein, the identifying of the subject as having a cell that has an elevated level of one or more of CRP mRNA, SAA mRNA, HP mRNA, ceruloplasmin mRNA, pro-IL-6 mRNA, calprotectin (S100A8) mRNA, pro-IL-8 mRNA, leukotriene B4 mRNA, and myeloperoxidase mRNA.

In some embodiments of any of the methods described herein, the identifying of the subject as having a cell that has an elevated level of NLRP3 inflammasome expression includes detecting the level of one or more of NLRP3 protein, ASC protein, procaspase-1 protein, and caspase-1 protein. In some embodiments of any of the methods described herein, the identifying the subject as also having a cell that has an elevated level of NLRP3 inflammasome expression includes detecting the level of one or more of NLRP3 mRNA, ASC mRNA, and procaspase-1 mRNA.

In some embodiments of any of the methods described herein, where the selected treatment includes a therapeutically effective amount of an NLRP3 antagonist and a therapeutically effective amount of an anti-TNFα agent, the NLRP3 antagonist and the anti-TNFα agent can be administered to the subject at substantially the same time (e.g., in different dosage forms or formulated together into a single dosage form). In some embodiments of any of the methods described herein, wherein the selected treatment includes a therapeutically effective amount of an NLRP3 antagonist and a therapeutically effective amount of an anti-TNFα agent, the subject can be first administered the NLRP3 antagonist and then be administered the anti-TNFα agent. In some embodiments of any of the methods described herein, wherein the selected treatment includes a therapeutically effective amount of an NLRP3 antagonist and a therapeutically effective amount of an anti-TNFα agent, the subject can be first administered the anti-TNFα agent and then be administered the NLRP3 antagonist.

In some embodiments of any of the methods described herein, the NLRP3 antagonist is a compound of any one of Formulas I-X, or a pharmaceutically acceptable salt, solvate, or co-crystal thereof, and is administered to a subject in need thereof, in combination with an anti-TNFα agent; preferably in combination with Infliximab, Etanercept, Certolizumab pegol, Golimumab or Adalimumab; more preferably in combination with Adalimumab; more preferably in combination with Adalimumab.

In some embodiments of any of the methods described herein, the subject has been diagnosed or identified as having an inflammatory or an autoimmune disorder (e.g., any of the exemplary inflammatory or autoimmune disorders described herein or known in the art). For example, in some embodiments of any of the methods described herein, the subject can be diagnosed or identified as having Crohn's disease, ulcerative colitis, or inflammatory bowel disease.

In some embodiments of any of the methods described herein, the anti-TNFα agent is an antibody or an antigen-binding antibody fragment (e.g., adalimumab, certolizumab, etanercept, golimumab, infliximab, CDP571, or certolizumab pegol), or a soluble TNFα receptor. In some embodiments of any of the methods described herein, the anti-TNFα agent is a small molecule inhibitor of a signaling component downstream of a TNFα receptor. In some embodiments of any of the methods described herein, the anti-TNFα agent is an inhibitory nucleic acid. In some embodiments of any of the methods described herein, the anti-TNFα agent is a fusion protein.

In some embodiments of any of the methods described herein, the NLRP3 antagonist is an inhibitory nucleic acid (e.g., a short interfering RNA, an antisense nucleic acid, or a ribozyme). In some embodiments of any of the methods described herein, the NLRP3 antagonist is a compound of any one of Formulas I-XII described herein, or a pharmaceutically acceptable salt, solvate, or co-crystal thereof. In some embodiments of any of the methods described herein, the NLRP3 antagonist is a compound shown in any one of Tables 1-18, or a pharmaceutically acceptable salt, solvate, or co-crystal thereof.

Some embodiments of any of the methods described herein can further include recording the selected treatment in the subject's clinical record (e.g., a computer readable medium). Some embodiments of any of the methods described herein can further include administering one or more doses (e.g., at least two, at least four, at least six, at least eight, at least ten doses) of the selected treatment to the identified subject.

Also provided herein are methods of selecting a subject (e.g., any of the exemplary subjects described herein) for treatment, that include: (a) identifying a subject having resistance to an anti-TNFα agent (e.g., any of the exemplary resistances to an anti-TNFα agent described herein or known in the art); and (b) selecting the identified subject for treatment with a therapeutically effective amount of an NLRP3 antagonist (e.g., any of the exemplary NLRP3 antagonists described herein or known in the art) or a pharmaceutically acceptable salt, solvate, or co-crystal thereof. In some embodiments of these methods, step (b) further includes identifying the subject as also having an elevated level of NLRP3 inflammasome activity and/or expression in a cell obtained from the subject, as compared to a reference level. In some embodiments of any of these methods, the treatment can further include a therapeutically effective amount of an anti-TNFα agent (e.g., any of the exemplary anti-TNFα agents described herein or known in the art), in addition to the NLRP3 antagonist.

Also provided herein are methods of selecting a subject (e.g., any of the exemplary subjects described herein) for treatment that include: selecting a subject identified as having resistance to an anti-TNFα agent (e.g., any of the exemplary resistances to an anti-TNFα agent described herein or known in the art), for treatment with a therapeutically effective level of an NLRP3 antagonist (e.g., any of the exemplary NLRP3 antagonists described herein or known in the art) or a pharmaceutically acceptable salt, solvate, or co-crystal thereof. In some embodiments of these methods, the identified subject also has an elevated level of NLRP3 inflammasome activity and/or expression in a cell obtained from the subject, as compared to a reference level. In some embodiments of these methods, the treatment further includes a therapeutically effective amount of an anti-TNFα agent (e.g., any of the exemplary anti-TNFα agents described herein or known in the art), in addition to the NLRP3 antagonist.

Also provided herein are methods of selecting a subject (e.g., any of the exemplary subjects described herein) for treatment, that include: (a) identifying a subject having an elevated level (e.g., an increase of 1% to 1000%, or any of the subranges of this range described herein) of NLRP3 inflammasome activity and/or expression in a cell obtained from the subject, as compared to a reference level (e.g., any of the exemplary reference levels of NLRP3 inflammasome activity described herein or known in the art); and (b) selecting the identified subject for treatment with (i) a therapeutically effective amount of an NLRP3 antagonist (e.g., any of the exemplary NLRP3 antagonists described herein or known in the art) or a pharmaceutically acceptable salt, solvate, or co-crystal thereof, and (ii) a therapeutically effective amount of an anti-TNFα agent (e.g., any of the exemplary anti-TNFα agents described herein or known in the art). In some embodiments of these methods, the subject has not previously been administered an anti-TNFα agent.

Also provided herein are methods of selecting a subject (e.g., any of the exemplary subjects described herein) for treatment, that include: selecting a subject identified as having an elevated level (e.g., an increase of 1% to 1000%, or any of the subranges of this range described herein) of NLRP3 inflammasome activity and/or expression in a cell obtained from the subject, as compared to a reference level (e.g., any of the exemplary reference levels of NLRP3 inflammasome activity described herein or known in the art), for treatment with (i) a therapeutically effective level of an NLRP3 antagonist (e.g., any of the exemplary NLRP3 antagonists described herein or known in the art) or a pharmaceutically acceptable salt, solvate, or co-crystal thereof, and (ii) a therapeutically effective amount of an anti-TNFα agent (e.g., any of the anti-TNFα agents described herein or known in the art). In some embodiments of these methods, the subject has not previously been administered an anti-TNFα agent.

In some embodiments of any of the methods described herein, the NLRP3 inflammasome activity is secretion of IL-18 (processed IL-18). In some embodiments of any of the methods described herein, the NLRP3 inflammasome activity is secretion of IL-1β (processed IL-1∂). In some embodiments of any of the methods described herein, the NLRP3 inflammasome activity is caspase-1 activity. In some embodiments of any of the methods described herein, the NLRP3 inflammasome activity is the level of lipocalin-2. In some embodiments of any of the methods described herein, the NLRP3 inflammasome activity is the level of S100A8. In some embodiments of any of the methods described herein, the NLRP3 inflammasome activity is the level of S100A9.

In some embodiments of any of the methods described herein, the identifying of the subject as having a cell that has an elevated level of NLRP3 inflammasome expression includes detecting the level of one or more of NLRP3 protein, ASC protein, procaspase-1 protein, and caspase-1 protein. In some embodiments of any of the methods described herein, the identifying the subject as also having a cell that has an elevated level of NLRP3 inflammasome expression includes detecting the level of one or more of NLRP3 mRNA, ASC mRNA, and procaspase-1 mRNA.

In some embodiments of any of the methods described herein, the identifying of the subject as having a cell that has an elevated level of NLRP3 inflammasome activity includes detecting the level of one or more of CRP protein, SAA protein, HP protein, ceruloplasmin protein, IL-6 protein (e.g., mature or pro-IL-6 protein), calprotectin (S100A8) protein, IL-8 protein (e.g., mature or pro-IL-8 protein), leukotriene B4 protein, and myeloperoxidase protein. In some embodiments of any of the methods described herein, the identifying of the subject as having a cell that has an elevated level of one or more of CRP mRNA, SAA mRNA, HP mRNA, ceruloplasmin mRNA, pro-IL-6 mRNA, calprotectin (S100A8) mRNA, pro-IL-8 mRNA, leukotriene B4 mRNA, and myeloperoxidase mRNA.

In some embodiments of any of the methods described herein, where the subject is selected for a treatment including (i) a therapeutically effective amount of an NLRP3 antagonist and (ii) a therapeutically effective amount of an anti-TNFα agent, the NLRP3 antagonist and the anti-TNFα agent can be administered at substantially the same time (e.g., in different dosage forms or formulated together into a single dosage form). In some embodiments of any of the methods described herein, where the subject is selected for a treatment including a therapeutically effective amount of an NLRP3 antagonist and a therapeutically effective amount of an anti-TNFα agent, the subject can be first administered the NLRP3 antagonist and then be administered the anti-TNFα agent. In some embodiments of any of the methods described herein, where the subject is selected for a treatment including a therapeutically effective amount of an NLRP3 antagonist and a therapeutically effective amount of an anti-TNFα agent, the subject can be first administered the anti-TNFα agent and then be administered the NLRP3 antagonist.

In some embodiments of any of the methods described herein, the subject has been diagnosed or identified as having an inflammatory or an autoimmune disorder (e.g., any of the exemplary inflammatory or autoimmune disorders described herein or known in the art). For example, in some embodiments of any of the methods described herein, the subject can be diagnosed or identified as having Crohn's disease, ulcerative colitis, or inflammatory bowel disease.

In some embodiments of any of the methods described herein, the anti-TNFα agent is an antibody or an antigen-binding antibody fragment (e.g., adalimumab, certolizumab, etanercept, golimumab, infliximab, CDP571, or certolizumab pegol), or a soluble TNFα receptor. In some embodiments of any of the methods described herein, the anti-TNFα agent is a small molecule inhibitor of a signaling component downstream of a TNFα receptor. In some embodiments of any of the methods described herein, the anti-TNFα agent is an inhibitory nucleic acid. In some embodiments of any of the methods described herein, the anti-TNFα agent is a fusion protein.

Additional exemplary aspects that can be used or incorporated in these methods are described herein.

In some embodiments of any of the methods described herein, the NLRP3 antagonist is an inhibitory nucleic acid (e.g., a short interfering RNA, an antisense nucleic acid, or a ribozyme). In some embodiments of any of the methods described herein, the NLRP3 antagonist is a compound of any one of Formulas I-X, or a pharmaceutically acceptable salt, solvate, or co-crystal thereof. In some embodiments of any of the methods described herein, the NLRP3 antagonist is a compound shown in any one of Tables 1-18, or a pharmaceutically acceptable salt, solvate, or co-crystal thereof.

Additional exemplary aspects that can be used or incorporated in these methods are described herein.

Methods of Reducing the Risk of Developing Resistance to an Anti-TNFα Agent

Provided herein are methods of reducing (e.g., a 1% to 99% decrease, or any of the subranges of this range described herein) the risk of developing resistance to an anti-TNFα agent in a subject (e.g., any of the exemplary subjects described herein) in need thereof, that include: administering to a subject in need thereof a therapeutically effective amount of an anti-TNFα agent (e.g., any of the exemplary anti-TNFα agents described herein or known in the art) and a therapeutically effective amount of an NLRP3 antagonist (e.g., any of the exemplary NLRP3 antagonists described herein or known in the art) or a pharmaceutically acceptable salt, solvate, or co-crystal thereof.

In some embodiments of any of the methods described herein, the anti-TNFα agent and the NLRP3 antagonist or a pharmaceutically acceptable salt, solvate, or co-crystal thereof are administered at substantially the same time. In some embodiments of these methods, the anti-TNFα agent and the NLRP3 antagonist or a pharmaceutically acceptable salt, solvate, or co-crystal thereof are formulated into a single dosage form.

In some embodiments of any of the methods described herein, the NLRP3 antagonist or a pharmaceutically acceptable salt, solvate, or co-crystal thereof is administered to the subject prior to administration of the anti-TNFα agent. In some embodiments of any of the methods described herein, the anti-TNFα agent is administered to the subject prior to administration of the NLRP3 antagonist or a pharmaceutically acceptable salt, solvate, or co-crystal thereof.

In some embodiments of any of the methods described herein, the subject has been diagnosed or identified as having an inflammatory or an autoimmune disorder (e.g., any of the exemplary inflammatory or autoimmune disorders described herein or known in the art). For example, in some embodiments of any of the methods described herein, the subject can be diagnosed or identified as having Crohn's disease, ulcerative colitis, or inflammatory bowel disease.

In some embodiments of any of the methods described herein, the anti-TNFα agent is an antibody or an antigen-binding antibody fragment (e.g., adalimumab, certolizumab, etanercept, golimumab, infliximab, CDP571, or certolizumab pegol), or a soluble TNFα receptor. In some embodiments of any of the methods described herein, the anti-TNFα agent is a small molecule inhibitor of a signaling component downstream of a TNFα receptor. In some embodiments of any of the methods described herein, the anti-TNFα agent is an inhibitory nucleic acid. In some embodiments of any of the methods described herein, the anti-TNFα agent is a fusion protein.

In some embodiments of any of the methods described herein, the NLRP3 antagonist is an inhibitory nucleic acid (e.g., a short interfering RNA, an antisense nucleic acid, or a ribozyme). In some embodiments of any of the methods described herein, the NLRP3 antagonist is a compound of any one of Formulas I-XII described herein, or a pharmaceutically acceptable salt, solvate, or co-crystal thereof. In some embodiments of any of the methods described herein, the NLRP3 antagonist is a compound shown in any one of Tables 1-18, or a pharmaceutically acceptable salt, solvate, or co-crystal thereof.

In some embodiments of any of the methods described herein, the NLRP3 antagonist is a compound of any one of Formulas I-X, or a pharmaceutically acceptable salt, solvate, or co-crystal thereof, and is administered to a subject in need thereof, in combination with an anti-TNFα agent; preferably in combination with Infliximab, Etanercept, Certolizumab pegol, Golimumab or Adalimumab; more preferably in combination with Adalimumab; more preferably in combination with Adalimumab.

Additional exemplary aspects that can be used or incorporated in these methods are described herein.

Methods of Detecting the Level of NLRP3 Inflammasome Activity and/or Expression

In some embodiments of any of the methods described herein, the NLRP3 inflammmasome activity is the secretion of IL-18 (processed IL-18). In some embodiments of any of the methods described herein, the NLPR3 inflammasome activity is the secretion of IL-1β (processed IL-1∂). In some embodiments of any of the methods described herein, the NLRP3 inflammasome activity is caspase-1 activity in a mammalian cell (e.g., a mammalian cell obtained from the subject). Non-limiting examples of methods that can be used to detect the secretion of IL-18 (processed IL-18) and IL-1β (processed IL-1∂) include immunohistochemistry, immunoassays, e.g., enzyme-linked immunosorbent assay (ELISA), sandwich ELISA, immunoprecipitation, and immunofluorescent assay. Non-limiting examples of commercially available assays for determining caspase-1 activity include Caspase 1 Assay Kit (Fluormetric) (ab39412) (Abcam), FAM-FLICA® Caspase-1 Assay Kit (ImmunoChemistry), Caspase-1 Colorimetric Assay Kit (K111) (Biovision, Inc.), and Caspase-1/ICE Colorimetric Assay Kit (R&D Systems).

In some embodiments of any of the methods described herein, the NLRP3 inflammasome activity can be the level of expression of an upstream activator of NLRP3 inflammasomes (e.g., the level of one or more (e.g., two, three, four, five, or six) of lipocalin-2 protein, lipocalin-2 mRNA, S100A8 protein, S1008A8 mRNA, S100A9 protein, or S100A9 mRNA) in a mammalian cell (e.g., a mammalian cell obtained from a subject). Non-limiting assays that can be used to determine NLRP3 activity include: Southern blot analysis, Norther blot analysis, polymerase chain reaction (PCR)-based methods, e.g., next generation sequencing, reverse transcription polymerase chain reaction (RT-PCR), TaqMan™, microarray analysis, immunohistochemistry, immunoassays, e.g., enzyme-linked immunosorbent assay (ELISA), sandwich ELISA, immunoprecipitation, immunofluorescent assay, and flow cytometry.

In some embodiments of any of the methods described herein, the NLRP3 inflammasome expression can be determined by detecting the level of one or more (e.g., two, three, four, five, six, or seven) of: NLRP3 protein, ASC protein, procaspase-1 protein, caspase-1 protein, NLRP3 mRNA, ASC mRNA, and procaspase-1 mRNA, in a mammalian cell (e.g., in a mammalian cell obtained from the subject). Non-limiting examples of assays that can be used to determine the level of NLRP3 protein, ASC protein, procaspase-1 protein, and caspase-1 protein include immunohistochemistry, immunoassays, e.g., enzyme-linked immunosorbent assay (ELISA), sandwich ELISA, immunoprecipitation, immunofluorescent assay, and flow cytometry. Non-limiting examples of assays that can be used to determine the level of NLRP3 mRNA, ASC mRNA, and procaspase-1 mRNA include Southern blot analysis, Norther blot analysis, polymerase chain reaction (PCR)-based methods, e.g., next generation sequencing, reverse transcription polymerase chain reaction (RT-PCR), TaqMan™, and microarray analysis.

In some embodiments of any of the methods described herein, the NLRP3 inflammasome expression can be determined by detecting the level of one or more of CRP protein, SAA protein, HP protein, ceruloplasmin protein, IL-6 protein (e.g., mature or pro-IL-6 protein), calprotectin (S100A8) protein, IL-8 protein (e.g., mature or pro-IL-8 protein), leukotriene B4 protein, myeloperoxidase protein, CRP mRNA, SAA mRNA, HP mRNA, ceruloplasmin mRNA, pro-IL-6 mRNA, calprotectin (S100A8) mRNA, pro-IL-8 mRNA, leukotriene B4 mRNA, and myeloperoxidase mRNA. Non-limiting examples of assays that can be used to determine the level of CRP protein, SAA protein, HP protein, ceruloplasmin protein, IL-6 protein (e.g., mature or pro-IL-6 protein), calprotectin (S100A8) protein, IL-8 protein (e.g., mature or pro-IL-8 protein), leukotriene B4 protein, myeloperoxidase protein include immunohistochemistry, immunoassays, e.g., enzyme-linked immunosorbent assay (ELISA), sandwich ELISA, immunoprecipitation, immunofluorescent assay, and flow cytometry. Non-limiting examples of assays that can be used to determine the level of CRP mRNA, SAA mRNA, HP mRNA, ceruloplasmin mRNA, pro-IL-6 mRNA, calprotectin (S100A8) mRNA, pro-IL-8 mRNA, leukotriene B4 mRNA, and myeloperoxidase mRNA include Southern blot analysis, Norther blot analysis, polymerase chain reaction (PCR)-based methods, e.g., next generation sequencing, reverse transcription polymerase chain reaction (RT-PCR), TaqMan™, and microarray analysis.

In some embodiments of any of the methods described herein, the level of the protein or mRNA can be detected in a biological sample including blood, serum, exosomes, plasma, tissue, urine, feces, sputum, and cerebrospinal fluid.

In some embodiments of any of the methods described herein, the level of at least one (e.g., 2, 3, 4, 5, 6, 7 or 8) NLRP3 inflammasome activity and/or expression can be determined, e.g., in any combination.

In one aspect, the cell can be a cell isolated from a subject who has been screened for the presence of an inflammatory disease or indication that is associated with a mutation in a NLRP3 activity.

Methods of Determining Resistance of an Anti-TNFα Agent

Resistance to an anti-TNFα agent (e.g., primary resistance) is a reduced or decreased level of sensitivity to treatment with an anti-TNFα agent in a subject (e.g., as compared to a similar subject or as compared to the level of sensitivity to the anti-TNFα agent at an earlier time point). For example, resistance to an anti-TNFα in a subject can be observed by a physician or trained medical professional, e.g., by observing the requirement of increasing dosage amounts of an anti-TNFα agent (e.g., any of the exemplary anti-TNFα agents described herein or known in the art) over time in order to achieve the same therapeutic effect in a subject (e.g., any of the exemplary subjects described herein), observing the requirement for an increased number of doses and/or an increased frequency of doses of an anti-TNFα agent (e.g., any of the exemplary anti-TNFα agents described herein or known in the art) over time in order to achieve the same therapeutic effect in a subject (e.g., any of the exemplary subjects described herein), a decrease in the observed therapeutic response to treatment with the same dosage of an anti-TNFα agent (e.g., any of the exemplary anti-TNFα agents described herein or known in the art) in a subject (e.g., any of the exemplary subjects described herein) over time, or an observed progression of disease or disease relapse (e.g., any of the inflammatory diseases or autoimmune diseases described herein) in a subject (e.g., any of the exemplary subjects described herein) administered an anti-TNFα agent (e.g., any of the exemplary anti-TNFα agents described herein or known in the art). Additional metrics and assessments of resistance to an anti-TNFα agent are known in the art.

Anti-TNFα Agents

The term “anti-TNFα agent” refers to an agent which directly or indirectly blocks, down-regulates, impairs, inhibits, impairs, or reduces TNFα activity and/or expression. In some embodiments, an anti-TNFα agent is an antibody or an antigen-binding fragment thereof, a fusion protein, a soluble TNFα receptor (a soluble tumor necrosis factor receptor superfamily member 1A (TNFR1) or a soluble tumor necrosis factor receptor superfamily 1B (TNFR2)), an inhibitory nucleic acid, or a small molecule TNFα antagonist. In some embodiments, the inhibitory nucleic acid is a ribozyme, small hairpin RNA, a small interfering RNA, an antisense nucleic acid, or an aptamer.

Exemplary anti-TNFα agents that directly block, down-regulate, impair, inhibit, or reduce TNFα activity and/or expression can, e.g., inhibit or decrease the expression level of TNFα or a receptor of TNFα (TNFR1 or TNFR2) in a cell (e.g., a cell obtained from a subject, a mammalian cell), or inhibit or reduce binding of TNFα to its receptor (TNFR1 and/or TNFR2). Non-limiting examples of anti-TNFα agents that directly block, down-regulate, impair, inhibit, or reduce TNFα activity and/or expression include an antibody or fragment thereof, a fusion protein, a soluble TNFα receptor (e.g., a soluble TNFR1 or soluble TNFR2), inhibitory nucleic acids (e.g., any of the examples of inhibitory nucleic acids described herein), and a small molecule TNFα antagonist.

Exemplary anti-TNFα agents that can indirectly block, down-regulate, impair, inhibit reduce TNFα activity and/or expression can, e.g., inhibit or decrease the level of downstream signaling of a TNFα receptor (e.g., TNFR1 or TNFR2) in a mammalian cell (e.g., decrease the level and/or activity of one or more of the following signaling proteins: AP-1, mitogen-activated protein kinase kinase kinase 5 (ASK1), inhibitor of nuclear factor kappa B (IKK), mitogen-activated protein kinase 8 (JNK), mitogen-activated protein kinase (MAPK), MEKK 1/4, MEKK 4/7, MEKK 3/6, nuclear factor kappa B (NF-κB), mitogen-activated protein kinase kinase kinase 14 (NIK), receptor interacting serine/threonine kinase 1 (RIP), TNFRSF1A associated via death domain (TRADD), and TNF receptor associated factor 2 (TRAF2), in a cell), and/or decrease the level of TNFα-induced gene expression in a mammalian cell (e.g., decrease the transcription of genes regulated by, e.g., one or more transcription factors selected from the group of activating transcription factor 2 (ATF2), c-Jun, and NF-κB). A description of downstream signaling of a TNFα receptor is provided in Wajant et al., Cell Death Differentiation 10:45-65, 2003 (incorporated herein by reference). For example, such indirect anti-TNFα agents can be an inhibitory nucleic acid that targets (decreases the expression) a signaling component downstream of a TNFα-induced gene (e.g., any TNFα-induced gene known in the art), a TNFα receptor (e.g., any one or more of the signaling components downstream of a TNFα receptor described herein or known in the art), or a transcription factor selected from the group of NF-κB, c-Jun, and ATF2.

In other examples, such indirect anti-TNFα agents can be a small molecule inhibitor of a protein encoded by a TNFα-induced gene (e.g., any protein encoded by a TNFα-induced gene known in the art), a small molecule inhibitor of a signaling component downstream of a TNFα receptor (e.g., any of the signaling components downstream of a TNFα receptor described herein or known in the art), and a small molecule inhibitor of a transcription factor selected from the group of ATF2, c-Jun, and NF-κB.

In other embodiments, anti-TNFα agents that can indirectly block, down-regulate, impair, or reduce one or more components in a cell (e.g., a cell obtained from a subject, a mammalian cell) that are involved in the signaling pathway that results in TNFα mRNA transcription, TNFα mRNA stabilization, and TNFα mRNA translation (e.g., one or more components selected from the group of CD14, c-Jun, ERK1/2, IKK, IκB, interleukin 1 receptor associated kinase 1 (IRAK), JNK, lipopolysaccharide binding protein (LBP), MEK1/2, MEK3/6, MEK4/7, MK2, MyD88, NF-κB, NIK, PKR, p38, AKT serine/threonine kinase 1 (rac), raf kinase (raf), ras, TRAF6, TTP). For example, such indirect anti-TNFα agents can be an inhibitory nucleic acid that targets (decreases the expression) of a component in a mammalian cell that is involved in the signaling pathway that results in TNFα mRNA transcription, TNFα mRNA stabilization, and TNFα mRNA translation (e.g., a component selected from the group of CD14, c-Jun, ERK1/2, IKK, IκB, IRAK, JNK, LBP, MEK1/2, MEK3/6, MEK4/7, MK2, MyD88, NF-κB, NIK, IRAK, lipopolysaccharide binding protein (LBP), PKR, p38, rac, raf, ras, TRAF6, TTP). In other examples, an indirect anti-TNFα agents is a small molecule inhibitor of a component in a mammalian cell that is involved in the signaling pathway that results in TNFα mRNA transcription, TNFα mRNA stabilization, and TNFα mRNA translation (e.g., a component selected from the group of CD14, c-Jun, ERK1/2, IKK, IκB, IRAK, JNK, lipopolysaccharide binding protein (LBP), MEK1/2, MEK3/6, MEK4/7, MK2, MyD88, NF-κB, NIK, IRAK, lipopolysaccharide binding protein (LBP), PKR, p38, rac, raf, ras, TRAF6, TTP).

### Antibodies

In some embodiments, the anti-TNFα agent is an antibody or an antigen-binding fragment thereof (e.g., a Fab or a scFv). In some embodiments, an antibody or antigen-binding fragment of an antibody described herein can bind specifically to TNFα. In some embodiments, an antibody or antigen-binding fragment described herein binds specifically to any one of TNFα, TNFR1, or TNFR2. In some embodiments, an antibody or antigen-binding fragment of an antibody described herein can bind specifically to a TNFα receptor (TNFR1 or TNFR2).

In some embodiments, the antibody can be a humanized antibody, a chimeric antibody, a multivalent antibody, or a fragment thereof. In some embodiments, an antibody can be a scFv-Fc, a VHH domain, a VNAR domain, a (scFv)2, a minibody, or a BiTE.

In some embodiments, an antibody can be a crossmab, a diabody, a scDiabody, a scDiabody-CH3, a Diabody-CH3, a DutaMab, a DT-IgG, a diabody-Fc, a scDiabody-HAS, a charge pair antibody, a Fab-arm exchange antibody, a SEEDbody, a Triomab, a LUZ-Y, a Fcab, a kλ-body, an orthogonal Fab, a DVD-IgG, an IgG(H)-scFv, a scFv-(H)IgG, an IgG(L)-scFv, a scFv-(L)-IgG, an IgG (L,H)-Fc, an IgG(H)-V, a V(H)-IgG, an IgG(L)-V, a V(L)-IgG, an KIH IgG-scFab, a 2scFv-IgG, an IgG-2scFv, a scFv4-Ig, a Zybody, a DVI-IgG, a nanobody, a nanobody-HSA, a DVD-Ig, a dual-affinity re-targeting antibody (DART), a triomab, a kih IgG with a common LC, an ortho-Fab IgG, a 2-in-1-IgG, IgG-ScFv, scFv2-Fc, a bi-nanobody, tanden antibody, a DART-Fc, a scFv-HAS-scFv, a DAF (two-in-one or four-in-one), a DNL-Fab3, knobs-in-holes common LC, knobs-in-holes assembly, a TandAb, a Triple Body, a miniantibody, a minibody, a TriBi minibody, a scFv-CH3 KIH, a Fab-scFv, a scFv-CH-CL-scFv, a F(ab′)2-scFV2, a scFv-KIH, a Fab-scFv-Fc, a tetravalent HCAb, a scDiabody-Fc, a tandem scFv-Fc, an intrabody, a dock and lock bispecific antibody, an ImmTAC, a HSAbody, a tandem scFv, an IgG-IgG, a Cov-X-Body, and a scFv1-PEG-scFv2.

Non-limiting examples of an antigen-binding fragment of an antibody include an Fv fragment, a Fab fragment, a F(ab′)2 fragment, and a Fab′ fragment. Additional examples of an antigen-binding fragment of an antibody is an antigen-binding fragment of an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); or an antigen-binding fragment of an IgM (e.g., an antigen-binding fragment of a human or humanized IgM).

Non-limiting examples of anti-TNFα agents that are antibodies that specifically bind to TNFα are described in Ben-Horin et al., Autoimmunity Rev. 13(1):24-30, 2014; Bongartz et al., JAMA 295(19):2275-2285, 2006; Butler et al., Eur. Cytokine Network 6(4):225-230, 1994; Cohen et al., Canadian J Gastroenterol. Hepatol. 15(6):376-384, 2001; Elliott et al., Lancet 1994; 344: 1125-1127, 1994; Feldmann et al., Ann. Rev. Immunol. 19(1):163-196, 2001; Rankin et al., Br. J Rheumatol. 2:334-342, 1995; Knight et al., Molecular Immunol. 30(16):1443-1453, 1993; Lorenz et al., J. Immunol. 156(4):1646-1653, 1996; Hinshaw et al., Circulatory Shock 30(3):279-292, 1990; Ordas et al., Clin. Pharmacol. Therapeutics 91(4):635-646, 2012; Feldman, Nature Reviews Immunol. 2(5):364-371, 2002; Taylor et al., Nature Reviews Rheumatol. 5(10):578-582, 2009; Garces et al., Annals Rheumatic Dis. 72(12):1947-1955, 2013; Palladino et al., Nature Rev. Drug Discovery 2(9):736-746, 2003; Sandborn et al., Inflammatory Bowel Diseases 5(2):119-133, 1999; Atzeni et al., Autoimmunity Reviews 12(7):703-708, 2013; Maini et al., Immunol. Rev. 144(1):195-223, 1995; Wanner et al., Shock 11(6):391-395, 1999; and U.S. Pat. Nos. 6,090,382; 6,258,562; and 6,509,015).

In certain embodiments, the anti-TNFα agent can include or is golimumab (Golimumab™), adalimumab (Humira™), infliximab (Remicade™), CDP571, CDP 870, or certolizumab pegol (Cimzia™). In certain embodiments, the anti-TNFα agent can be a TNFα inhibitor biosimilar. Examples of approved and late-phase TNFα inhibitor biosimilars include, but are not limited to, infliximab biosimilars such as Flixabi™ (SB2) from Samsung Bioepis, Inflectra® (CT-P13) from Celltrion/Pfizer, GS071 from Aprogen, Remsima™, PF-06438179 from Pfizer/Sandoz, NI-071 from Nichi-Iko Pharmaceutical Co., and ABP 710 from Amgen; adalimumab biosimilars such as Amgevita® (ABP 501) from Amgen and Exemptia™ from Zydus Cadila, BMO-2 or MYL-1401-A from Biocon/Mylan, CHS-1420 from Coherus, FKB327 from Kyowa Kirin, and BI 695501 from Boehringer Ingelheim; Solymbic®, SB5 from Samsung Bioepis, GP-2017 from Sandoz, ONS-3010 from Oncobiologics, M923 from Momenta, PF-06410293 from Pfizer, and etanercept biosimilars such as Erelzi™ from Sandoz/Novartis, Brenzys™ (SB4) from Samsung Bioepis, GP2015 from Sandoz, TuNEX® from Mycenax, LBEC0101 from LG Life, and CHS-0214 from Coherus.

In some embodiments of any of the methods described herein, the anti-TNFα agent is selected from the group consisting of: adalimumab, certolizumab, etanercept, golimumab, infliximabm, CDP571, and CDP 870.

In some embodiments, any of the antibodies or antigen-binding fragments described herein has a dissociation constant (K_{D}) of less than 1×10⁻⁵ M (e.g., less than 0.5×10⁻⁵ M, less than 1×10⁻⁶ M, less than 0.5×10⁻⁶ M, less than 1×10⁻⁶ M, less than 0.5×10⁻⁷M, less than 1×10⁻⁸M, less than 0.5×10⁻⁸M, less than 1×10⁻⁶ M, less than 0.5×10⁻⁹ M, less than 1×10⁻¹⁰ M, less than 0.5×10⁻¹⁰ M, less than 1×10⁻¹¹M, less than 0.5×10⁻⁶ M, or less than 1×10¹²M), e.g., as measured in phosphate buffered saline using surface plasmon resonance (SPR).

In some embodiments, any of the antibodies or antigen-binding fragments described herein has a K_{D} of about 1×10¹² M to about 1×10⁻⁵ M, about 0.5×10⁻⁵ M, about 1×10⁻⁶ M, about 0.5×10⁻⁶ M, about 1×10⁻⁶ M, about 0.5×10⁻⁶ M, about 1×10⁻⁸ M, about 0.5×10⁻⁸M, about 1×10⁻⁹ M, about 0.5×10⁻⁹ M, about 1×10⁻¹⁰ M, about 0.5×10⁻¹⁰ M, about 1×10⁻¹¹M, or about 0.5×10⁻¹¹ M (inclusive); about 0.5×10⁻¹¹ M to about 1×10⁻⁵ M, about 0.5×10⁻⁵ M, about 1×10⁻⁶ M, about 0.5×10⁻⁶ M, about 1×10⁻⁶ M, about 0.5×10⁻⁶ M, about 1×10⁻⁸M, about 0.5×10⁻⁸M, about 1×10⁻⁹ M, about 0.5×10⁻⁹ M, about 1×10⁻¹⁰ M, about 0.5×10⁻¹⁰ M, or about 1×10⁻¹¹M (inclusive); about 1×10⁻¹¹M to about 1×10⁻⁵ M, about 0.5×10⁻⁵ M, about 1×10⁻⁶ M, about 0.5×10⁻⁶ M, about 1×10⁻⁶ M, about 0.5×10⁻⁷M, about 1×10⁻⁸M, about 0.5×10⁻⁸M, about 1×10⁻⁹ M, about 0.5×10⁻⁹ M, about 1×10⁻¹⁰ M, or about 0.5×10⁻¹⁰ M (inclusive); about 0.5×10⁻¹⁰ M to about 1×10⁻⁵ M, about 0.5×10⁻⁵ M, about 1×10⁻⁶ M, about 0.5×10⁻⁶ M, about 1×10⁻⁶ M, about 0.5×10⁻⁶ M, about 1×10⁻⁸M, about 0.5×10⁻⁸M, about 1×10⁻⁹ M, about 0.5×10⁻⁹ M, or about 1×10⁻¹⁰ M (inclusive); about 1×10⁻¹⁰ M to about 1×10⁻⁵ M, about 0.5×10⁻⁵ M, about 1×10⁻⁶ M, about 0.5×10⁻⁶ M, about 1×10⁻⁶ M, about 0.5×10⁻⁷M, about 1×10⁻⁸M, about 0.5×10⁻⁸M, about 1×10⁻⁹ M, or about 0.5×10⁻⁹ M (inclusive); about 0.5×10⁻⁹ M to about 1×10⁻⁵ M, about 0.5×10⁻⁵ M, about 1×10⁻⁶ M, about 0.5×10⁻⁶ M, about 1×10⁻⁷M, about 0.5×10⁻⁷M, about 1×10⁻⁸M, about 0.5×10⁻⁸M, or about 1×10⁻⁹ M (inclusive); about 1×10⁻⁹ M to about 1×10⁻⁵ M, about 0.5×10⁻⁵ M, about 1×10⁻⁶ M, about 0.5×10⁻⁶ M, about 1×10⁻⁶ M, about 0.5×10⁻⁶ M, about 1×10⁻⁸M, or about 0.5×10⁻⁸M (inclusive); about 0.5×10⁻⁸M to about 1×10⁻⁵ M, about 0.5×10⁻⁵ M, about 1×10⁻⁶ M, about 0.5×10⁻⁶ M, about 1×10⁻⁶ M, about 0.5×10⁻⁷M, or about 1×10⁻⁸M (inclusive); about 1×10⁻⁸M to about 1×10⁻⁵ M, about 0.5×10⁻⁵ M, about 1×10⁻⁶ M, about 0.5×10⁻⁶ M, about 1×10⁻⁶ M, or about 0.5×10⁻⁶ M (inclusive); about 0.5×10⁻⁶ M to about 1×10⁻⁵ M, about 0.5×10⁻⁵ M, about 1×10⁻⁶ M, about 0.5×10⁻⁶ M, or about 1×10⁻⁷M (inclusive); about 1×10⁻⁶ M to about 1×10⁻⁵ M, about 0.5×10⁻⁵ M, about 1×10⁻⁶ M, or about 0.5×10⁻⁶ M (inclusive); about 0.5×10⁻⁶ M to about 1×10⁻⁵ M, about 0.5×10⁻⁵ M, or about 1×10⁻⁶ M (inclusive); about 1×10⁻⁶ M to about 1×10⁻⁵ M or about 0.5×10⁻⁵ M (inclusive); or about 0.5×10⁻⁵ M to about 1×10⁻⁵ M (inclusive), e.g., as measured in phosphate buffered saline using surface plasmon resonance (SPR).

In some embodiments, any of the antibodies or antigen-binding fragments described herein has a K_{off} of about 1×10's⁻¹ to about 1×10⁻³ s⁻¹, about 0.5×10⁻³ s⁻¹, about 1×10's⁻¹, about 0.5×10's⁻¹, about 1×10⁻⁵ s⁻¹, or about 0.5×10⁻⁵ s⁻¹ (inclusive); about 0.5×10⁻⁵ s⁻¹ to about 1×10⁻³ s⁻¹, about 0.5×10⁻³ s⁻¹, about 1×10's⁻¹, about 0.5×10's⁻¹, or about 1×10⁻⁵ s⁻¹ (inclusive); about 1×10⁻⁵ s⁻¹ to about 1×10⁻³ s⁻¹, about 0.5×10⁻³ s⁻¹, about 1×10's⁻¹, or about 0.5×10's⁻¹ (inclusive); about 0.5×10⁻⁴ s⁻¹ to about 1×10⁻³ s⁻¹, about 0.5×10⁻³ s⁻¹, or about 1×10's⁻¹ (inclusive); about 1×10's⁻¹ to about 1×10⁻³ s⁻¹, or about 0.5×10⁻³ s⁻¹ (inclusive); or about 0.5×10⁻⁵ s⁻¹ to about 1×10⁻³ s⁻¹ (inclusive), e.g., as measured in phosphate buffered saline using surface plasmon resonance (SPR).

In some embodiments, any of the antibodies or antigen-binding fragments described herein has a Kₒₙ of about 1×10²M⁻¹ s⁻¹ to about 1×10⁶M⁻¹ s⁻¹, about 0.5×10⁶ M⁻¹ s⁻¹, about 1×10⁵M⁻¹ s⁻¹, about 0.5×10⁵M⁻¹ s⁻¹, about 1×10⁴M⁻¹ s⁻¹, about 0.5×10⁴ M⁻¹ s⁻¹, about 1×10³M⁻¹ s⁻¹, or about 0.5×10³ M⁻¹ s⁻¹ (inclusive); about 0.5×10³ M⁻¹ s⁻¹ to about 1×10⁶M⁻¹ s⁻¹, about 0.5×10⁶M⁻¹ s⁻¹, about 1×10⁵M⁻¹ s⁻¹, about 0.5×10⁵M⁻¹ s⁻¹, about 1×10⁴M⁻¹ s⁻¹, about 0.5×10⁴M⁻¹ s⁻¹, or about 1×10³M⁻¹ s⁻¹ (inclusive); about 1×10³ M⁻¹ s⁻¹ to about 1×10⁶M⁻¹ s⁻¹, about 0.5×10⁶M⁻¹ s⁻¹, about 1×10⁵M⁻¹ s⁻¹, about 0.5×10⁵ M⁻¹ s⁻¹, about 1×10⁴ M⁻¹ s⁻¹, or about 0.5×10⁴ M⁻¹ s⁻¹ (inclusive); about 0.5×10⁴ M⁻¹ s⁻¹ to about 1×10⁶M⁻¹ s⁻¹, about 0.5×10⁶ M⁻¹ s⁻¹, about 1×10⁵M⁻¹ s⁻¹, about 0.5×10⁵ M⁻¹ s⁻¹, or about 1×10⁴ M⁻¹ s⁻¹ (inclusive); about 1×10⁴ M⁻¹ s⁻¹ to about 1×10⁶M⁻¹ s⁻¹, about 0.5×10⁶ M⁻¹ s⁻¹, about 1×10⁵M⁻¹ s⁻¹, or about 0.5×10⁵ M⁻¹ s⁻¹ (inclusive); about 0.5×10⁵ M⁻¹ s⁻¹ to about 1×10⁶M⁻¹ s⁻¹, about 0.5×10⁶ M⁻¹ s⁻¹, or about 1×10⁵M⁻¹ s⁻¹ (inclusive); about 1×10⁵M⁻¹ s⁻¹ to about 1×10⁶M⁻¹ s⁻¹, or about 0.5×10⁶ M⁻¹ s⁻¹ (inclusive); or about 0.5×10⁶ M⁻¹ s⁻¹ to about 1×10⁶M⁻¹ s⁻¹ (inclusive), e.g., as measured in phosphate buffered saline using surface plasmon resonance (SPR).

### Fusion Proteins

In some embodiments, the anti-TNFα agent is a fusion protein (e.g., an extracellular domain of a TNFR fused to a partner peptide, e.g., an Fc region of an immunoglobulin, e.g., human IgG) (see, e.g., Deeg et al., Leukemia 16(2):162, 2002; Peppel et al., J. Exp. Med. 174(6):1483-1489, 1991) or a soluble TNFR (e.g., TNFR1 or TNFR2) that binds specifically to TNFα. In some embodiments, the anti-TNFα agent includes or is a soluble TNFα receptor (e.g., Bjornberg et al., Lymphokine Cytokine Res. 13(3):203-211, 1994; Kozak et al., Am. J. Physiol. Reg. Integrative Comparative Physiol. 269(1):R23-R29, 1995; Tsao et al., Eur Respir J. 14(3):490-495, 1999; Watt et al., J Leukoc Biol. 66(6):1005-1013, 1999; Mohler et al., J Immunol. 151(3):1548-1561, 1993; Nophar et al., EMBO J. 9(10):3269, 1990; Piguet et al., Eur. Respiratory J. 7(3):515-518, 1994; and Gray et al., Proc. Natl. Acad. Sci. U.S.A. 87(19):7380-7384, 1990). In some embodiments, the anti-TNFα agent includes or is etanercept (Enbrel™) (see, e.g., WO 91/03553 and WO 09/406,476, incorporated by reference herein). In some embodiments, the anti-TNFα agent inhibitor includes or is r-TBP-I (e.g., Gradstein et al., J. Acquir. Immune Defic. Syndr. 26(2): 111-117, 2001).

### Inhibitory Nucleic Acids

Inhibitory nucleic acids that can decrease the expression of AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA expression in a mammalian cell include antisense nucleic acid molecules, i.e., nucleic acid molecules whose nucleotide sequence is complementary to all or part of a AP-1, ASK1, CD14, c-jun, ERK1/2, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA (e.g., complementary to all or a part of any one of SEQ ID NOs: 36-72).

The sequences characterized by the Sequences ID NO: 36-72 are listed below and are being submitted in a separate and machine readable file.
Human TNFα CDS (SEQ ID NO: 36), Human TNFR1 CDS (SEQ ID NO: 37), Human TNFR2 CDS (SEQ ID NO: 38), Human TRADD CDS (SEQ ID NO: 39), Human TRAF2 CDS (SEQ ID NO: 40), Human AP-1 CDS (SEQ ID NO: 41), Human ASK1 CDS (SEQ ID NO: 42), Human CD14 CDS (SEQ ID NO: 43), Human ERK1 CDS (SEQ ID NO: 44), Human ERK2 CDS (SEQ ID NO: 45), Human IKK CDS (SEQ ID NO: 46), Human KB CDS (SEQ ID NO: 47), Human IRAK CDS (SEQ ID NO: 48), Human JNK CDS (SEQ ID NO: 49), Human LBP CDS (SEQ ID NO: 50), Human MEK1 CDS (SEQ ID NO: 51), Human MEK2 CDS (SEQ ID NO: 52), Human MEK3 CDS (SEQ ID NO: 53), Human MEK6 CDS (SEQ ID NO: 54), Human MEKK1 CDS (SEQ ID NO: 55), Human MEKK 3 CDS (SEQ ID NO: 56), Human MEKK4 CDS (SEQ ID NO: 57), Human MEKK 6 CDS (SEQ ID NO: 58), Human MEKK7 CDS (SEQ ID NO: 59), Human MK2 CDS (SEQ ID NO: 60), Human MyD88 CDS (SEQ ID NO: 61), Human NF-κB CDS (SEQ ID NO: 62), Human NIK CDS (SEQ ID NO: 63), Human p38 CDS (SEQ ID NO: 64), Human PKR CDS (SEQ ID NO: 65), Human Rac CDS (SEQ ID NO: 66), Human Raf CDS (SEQ ID NO: 67), Human K-Ras CDS (SEQ ID NO: 68), Human N-Ras CDS (SEQ ID NO: 69), Human RIP CDS (SEQ ID NO: 70), Human TRAF6 CDS (SEQ ID NO: 71), and Human TTP CDS (SEQ ID NO: 72). An antisense nucleic acid molecule can be complementary to all or part of a non-coding region of the coding strand of a nucleotide sequence encoding an AP-1, ASK1, CD14, c-jun, ERK1/2, IxB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTPMEKK1 protein. Non-coding regions (5′ and 3′ untranslated regions) are the 5′ and 3′ sequences that flank the coding region in a gene and are not translated into amino acids.

Based upon the sequences disclosed herein, one of skill in the art can easily choose and synthesize any of a number of appropriate antisense nucleic acids to target a nucleic acid encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein described herein. Antisense nucleic acids targeting a nucleic acid encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTPMEKK1 protein can be designed using the software available at the Integrated DNA Technologies website.

An antisense nucleic acid can be, for example, about 5, 10, 15, 18, 20, 22, 24, 25, 26, 28, 30, 32, 35, 36, 38, 40, 42, 44, 45, 46, 48, or 50 nucleotides or more in length. An antisense oligonucleotide can be constructed using enzymatic ligation reactions and chemical synthesis using procedures known in the art. For example, an antisense nucleic acid can be chemically synthesized using variously modified nucleotides or naturally occurring nucleotides designed to increase the physical stability of the duplex formed between the antisense and sense nucleic acids, e.g., phosphorothioate derivatives and acridine substituted nucleotides or to increase the biological stability of the molecules.

Examples of modified nucleotides which can be used to generate an antisense nucleic acid include 1-methylguanine, 1-methylinosine, 2,2-dimethylguanine, 2-methyladenine, 2-methylguanine, 3-methylcytosine, 2-methylthio-N6-isopentenyladenine, uracil-5-oxyacetic acid (v), wybutoxosine, pseudouracil, queosine, 2-thiocytosine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules described herein can be prepared in vitro and administered to a subject, e.g., a human subject. Alternatively, they can be generated in situ such that they hybridize with or bind to cellular mRNA and/or genomic DNA encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein to thereby inhibit expression, e.g., by inhibiting transcription and/or translation. The hybridization can be by conventional nucleotide complementarities to form a stable duplex, or, for example, in the case of an antisense nucleic acid molecule that binds to DNA duplexes, through specific interactions in the major groove of the double helix. The antisense nucleic acid molecules can be delivered to a mammalian cell using a vector (e.g., an adenovirus vector, a lentivirus, or a retrovirus).

An antisense nucleic acid can be an α-anomeric nucleic acid molecule. An α-anomeric nucleic acid molecule forms specific double-stranded hybrids with complementary RNA in which, contrary to the usual, β-units, the strands run parallel to each other (Gaultier et al., Nucleic Acids Res. 15:6625-6641, 1987). The antisense nucleic acid can also comprise a chimeric RNA-DNA analog (Inoue et al., FEBS Lett. 215:327-330, 1987) or a 2′-O-methylribonucleotide (Inoue et al., Nucleic Acids Res. 15:6131-6148, 1987).

Another example of an inhibitory nucleic acid is a ribozyme that has specificity for a nucleic acid encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA, e.g., specificity for any one of SEQ ID NOs: 36-72). Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature 334:585-591, 1988)) can be used to catalytically cleave mRNA transcripts to thereby inhibit translation of the protein encoded by the mRNA. An AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA can be used to select a catalytic RNA having a specific ribonuclease activity from a pool of RNA molecules. See, e.g., Bartel et al., Science 261:1411-1418, 1993.

Alternatively, a ribozyme having specificity for an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA can be designed based upon the nucleotide sequence of any of the AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA sequences disclosed herein. For example, a derivative of a Tetrahymena L-19 IVS RNA can be constructed in which the nucleotide sequence of the active site is complementary to the nucleotide sequence to be cleaved in an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA (see, e.g., U.S. Pat. Nos. 4,987,071 and 5,116,742).

An inhibitory nucleic acid can also be a nucleic acid molecule that forms triple helical structures. For example, expression of an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP polypeptide can be inhibited by targeting nucleotide sequences complementary to the regulatory region of the gene encoding the AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP polypeptide (e.g., the promoter and/or enhancer, e.g., a sequence that is at least 1 kb, 2 kb, 3 kb, 4 kb, or 5 kb upstream of the transcription initiation start state) to form triple helical structures that prevent transcription of the gene in target cells. See generally Maher, Bioassays 14(12):807-15, 1992; Helene, Anticancer Drug Des. 6(6):569-84, 1991; and Helene, Ann. N.Y. Acad. Sci. 660:27-36, 1992.

In various embodiments, inhibitory nucleic acids can be modified at the sugar moiety, the base moiety, or phosphate backbone to improve, e.g., the solubility, stability, or hybridization, of the molecule. For example, the deoxyribose phosphate backbone of the nucleic acids can be modified to generate peptide nucleic acids (see, e.g., Hyrup et al., Bioorganic Medicinal Chem. 4(1):5-23, 1996). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to RNA and DNA under conditions of low ionic strength. PNA oligomers can be synthesized using standard solid phase peptide synthesis protocols (see, e.g., Perry-O'Keefe et al., Proc. Natl. Acad. Sci. U.S.A. 93:14670-675, 1996). PNAs can be used as antisense or antigene agents for sequence-specific modulation of gene expression by, e.g., inducing transcription or translation arrest or inhibiting replication.

### Small Molecules

In some embodiments, the anti-TNFα agent is a small molecule. In some embodiments, the small molecule is a tumor necrosis factor-converting enzyme (TACE) inhibitor (e.g., Moss et al., Nature Clinical Practice Rheumatology 4: 300-309, 2008). In some embodiments, the anti-TNFα agent is C87 (Ma et al., J. Biol. Chem. 289(18):12457-66, 2014). In some embodiments, the small molecule is LMP-420 (e.g., Haraguchi et al., AIDS Res. Ther. 3:8, 2006). In some embodiments, the TACE inhibitor is TMI-005 and BMS-561392. Additional examples of small molecule inhibitors are described in, e.g., He et al., Science 310(5750):1022-1025, 2005.

In some examples, the anti-TNFα agent is a small molecule that inhibits the activity of one of AP-1, ASK1, IKK, JNK, MAPK, MEKK 1/4, MEKK4/7, MEKK 3/6, NIK, TRADD, RIP, NF-κB, and TRADD in a cell (e.g., in a cell obtained from a subject, a mammalian cell).

In some examples, the anti-TNFα agent is a small molecule that inhibits the activity of one of CD14, MyD88 (see, e.g., Olson et al., Scientific Reports 5:14246, 2015), ras (e.g., Baker et al., Nature 497:577-578, 2013), raf (e.g., vemurafenib (PLX4032, RG7204), sorafenib tosylate, PLX-4720, dabrafenib (GSK2118436), GDC-0879, RAF265 (CHIR-265), AZ 628, NVP-BHG712, SB590885, ZM 336372, sorafenib, GW5074, TAK-632, CEP-32496, encorafenib (LGX818), CCT196969, LY3009120, R05126766 (CH5126766), PLX7904, and MLN2480).

In some examples, the anti-TNFα agent TNFα inhibitor is a small molecule that inhibits the activity of one of MK2 (PF 3644022 and PHA 767491), JNK (e.g., AEG 3482, BI 78D3, CEP 1347, c-JUN peptide, IQ 15, JIP-1 (153-163), SP600125, SU 3327, and TCS JNK6o), c-jun (e.g., AEG 3482, BI 78D3, CEP 1347, c-JUN peptide, IQ 15, JIP-1 (153-163), SP600125, SU 3327, and TCS JNK6o), MEK3/6 (e.g., Akinleye et al., J. Hematol. Oncol. 6:27, 2013), p38 (e.g., AL 8697, AMG 548, BIRB 796, CMPD-1, DBM 1285 dihydrochloride, EO 1428, JX 401, ML 3403, Org 48762-0, PH 797804, RWJ 67657, SB 202190, SB 203580, SB 239063, SB 706504, SCIO 469, SKF 86002, SX 011, TA 01, TA 02, TAK 715, VX 702, and VX 745), PKR (e.g., 2-aminopurine or CAS 608512-97-6), TTP (e.g., CAS 329907-28-0), MEK1/2 (e.g., Facciorusso et al., Expert Review Gastroentrol. Hepatol. 9:993-1003, 2015), ERK1/2 (e.g., Mandal et al., Oncogene 35:2547-2561, 2016), NIK (e.g., Mortier et al., Bioorg. Med. Chem. Lett. 20:4515-4520, 2010), IKK (e.g., Reilly et al., Nature Med. 19:313-321, 2013), IκB (e.g., Suzuki et al., Expert. Opin. Invest. Drugs 20:395-405, 2011), NF-κB (e.g., Gupta et al., Biochim. Biophys. Acta 1799(10-12):775-787, 2010), rac (e.g., U.S. Pat. No. 9,278,956), MEK4/7, IRAK (Chaudhary et al., J. Med. Chem. 58(1):96-110, 2015), LBP (see, e.g., U.S. Pat. No. 5,705,398), and TRAF6 (e.g., 3-[(2,5-Dimethylphenyl)amino]-1-phenyl-2-propen-1-one).

In some embodiments of any of the methods described herein, the inhibitory nucleic acid can be about 10 nucleotides to about 50 nucleotides (e.g., about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 28 nucleotides, about 10 nucleotides to about 26 nucleotides, about 10 nucleotides to about 25 nucleotides, about 10 nucleotides to about 24 nucleotides, about 10 nucleotides to about 22 nucleotides, about 10 nucleotides to about 20 nucleotides, about 10 nucleotides to about 18 nucleotides, about 10 nucleotides to about 16 nucleotides, about 10 nucleotides to about 14 nucleotides, about 10 nucleotides to about 12 nucleotides, about 12 nucleotides to about 50 nucleotides, about 12 nucleotides to about 45 nucleotides, about 12 nucleotides to about 40 nucleotides, about 12 nucleotides to about 35 nucleotides, about 12 nucleotides to about 30 nucleotides, about 12 nucleotides to about 28 nucleotides, about 12 nucleotides to about 26 nucleotides, about 12 nucleotides to about 25 nucleotides, about 12 nucleotides to about 24 nucleotides, about 12 nucleotides to about 22 nucleotides, about 12 nucleotides to about 20 nucleotides, about 12 nucleotides to about 18 nucleotides, about 12 nucleotides to about 16 nucleotides, about 12 nucleotides to about 14 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 28 nucleotides, about 15 nucleotides to about 26 nucleotides, about 15 nucleotides to about 25 nucleotides, about 15 nucleotides to about 24 nucleotides, about 15 nucleotides to about 22 nucleotides, about 15 nucleotides to about 20 nucleotides, about 15 nucleotides to about 18 nucleotides, about 15 nucleotides to about 16 nucleotides, about 16 nucleotides to about 50 nucleotides, about 16 nucleotides to about 45 nucleotides, about 16 nucleotides to about 40 nucleotides, about 16 nucleotides to about 35 nucleotides, about 16 nucleotides to about 30 nucleotides, about 16 nucleotides to about 28 nucleotides, about 16 nucleotides to about 26 nucleotides, about 16 nucleotides to about 25 nucleotides, about 16 nucleotides to about 24 nucleotides, about 16 nucleotides to about 22 nucleotides, about 16 nucleotides to about 20 nucleotides, about 16 nucleotides to about 18 nucleotides, about 18 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 28 nucleotides, about 20 nucleotides to about 26 nucleotides, about 20 nucleotides to about 25 nucleotides, about 20 nucleotides to about 24 nucleotides, about 20 nucleotides to about 22 nucleotides, about 24 nucleotides to about 50 nucleotides, about 24 nucleotides to about 45 nucleotides, about 24 nucleotides to about 40 nucleotides, about 24 nucleotides to about 35 nucleotides, about 24 nucleotides to about 30 nucleotides, about 24 nucleotides to about 28 nucleotides, about 24 nucleotides to about 26 nucleotides, about 24 nucleotides to about 25 nucleotides, about 26 nucleotides to about 50 nucleotides, about 26 nucleotides to about 45 nucleotides, about 26 nucleotides to about 40 nucleotides, about 26 nucleotides to about 35 nucleotides, about 26 nucleotides to about 30 nucleotides, about 26 nucleotides to about 28 nucleotides, about 28 nucleotides to about 50 nucleotides, about 28 nucleotides to about 45 nucleotides, about 28 nucleotides to about 40 nucleotides, about 28 nucleotides to about 35 nucleotides, about 28 nucleotides to about 30 nucleotides, about 30 nucleotides to about 50 nucleotides, about 30 nucleotides to about 45 nucleotides, about 30 nucleotides to about 40 nucleotides, about 30 nucleotides to about 38 nucleotides, about 30 nucleotides to about 36 nucleotides, about 30 nucleotides to about 34 nucleotides, about 30 nucleotides to about 32 nucleotides, about 32 nucleotides to about 50 nucleotides, about 32 nucleotides to about 45 nucleotides, about 32 nucleotides to about 40 nucleotides, about 32 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, about 42 nucleotides to about 50 nucleotides, about 42 nucleotides to about 45 nucleotides, or about 45 nucleotides to about 50 nucleotides) in length. One skilled in the art will appreciate that inhibitory nucleic acids may comprises at least one modified nucleic acid at either the 5′ or 3′ end of DNA or RNA.

In some embodiments, the inhibitory nucleic acid can be formulated in a liposome, a micelle (e.g., a mixed micelle), a nanoemulsion, or a microemulsion, a solid nanoparticle, or a nanoparticle (e.g., a nanoparticle including one or more synthetic polymers). Additional exemplary structural features of inhibitory nucleic acids and formulations of inhibitory nucleic acids are described in US 2016/0090598.

In some embodiments, the inhibitory nucleic acid (e.g., any of the inhibitory nucleic acid described herein) can include a sterile saline solution (e.g., phosphate-buffered saline (PBS)). In some embodiments, the inhibitory nucleic acid (e.g., any of the inhibitory nucleic acid described herein) can include a tissue-specific delivery molecule (e.g., a tissue-specific antibody).

### Indications

In some embodiments, methods for treating a subject having condition, disease or disorder in which a decrease or increase in NLRP3 activity (e.g., an increase, e.g., NLRP3 signaling) contributes to the pathology and/or symptoms and/or progression of the condition, disease or disorder are provided, comprising administering to a subject an effective amount of a chemical entity described herein (e.g., a compound described generically or specifically herein or a pharmaceutically acceptable salt thereof or compositions containing the same). In some embodiments of any of the methods described herein, the subject can have, or be diagnosed or identified as having, an inflammatory disease or an autoimmune disease. In some embodiments, the inflammatory disease or autoimmune disease. In some embodiments of any of the methods described herein, the subject can have, or be identified or diagnosed as having, any of the conditions, diseases, or disorders in which a decrease or increase in NLRP3 activity contributes to the pathology and/or symptoms and/or progression of the condition, disease, or disorder. In some embodiments of any of the methods described herein, the subject can be suspected of having or present with one or more symptoms of any of the conditions, diseases, or disorders described herein.

In some embodiments of any of the methods described herein, the subject can have, or be diagnosed or identified as having, an inflammatory disease or an autoimmune disease. In some embodiments, the condition, disease or disorder is selected from: inappropriate host responses to infectious diseases where active infection exists at any body site, such as septic shock, disseminated intravascular coagulation, and/or adult respiratory distress syndrome; acute or chronic inflammation due to antigen, antibody and/or complement deposition; inflammatory conditions including arthritis, cholangitis, colitis, encephalitis, endocarditis, glomerulonephritis, hepatitis, myocarditis, pancreatitis, pericarditis, reperfusion injury and vasculitis, immune-based diseases such as acute and delayed hypersensitivity, graft rejection, and graft-versus-host disease; autoimmune diseases including Type 1 diabetes mellitus and multiple sclerosis. For example, the condition, disease or disorder may be an inflammatory disorder such as rheumatoid arthritis, osteoarthritis, septic shock, COPD and periodontal disease.

In some embodiments, the condition, disease or disorder is an autoimmune diseases. Non-limiting examples include rheumatoid arthritis, systemic lupus erythematosus, multiple sclerosis, inflammatory bowel diseases (IBDs) comprising Crohn disease (CD) and ulcerative colitis (UC), which are chronic inflammatory conditions with polygenic susceptibility. In certain embodiments, the condition is an inflammatory bowel disease. In certain embodiments, the condition is Crohn's disease, autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease), radiation enteritis, collagenous colitis, lymphocytic colitis, microscopic colitis, and radiation enteritis. In certain of these embodiments, the condition is alloimmune disease (such as graft-vs-host disease, e.g., acute graft vs. host disease and chronic graft vs. host disease), celiac disease, inflammatory bowel syndrome, rheumatoid arthritis, lupus, scleroderma, psoriasis, cutaneous T-cell lymphoma, uveitis, and mucositis (e.g., oral mucositis, esophageal mucositis or intestinal mucositis).

In some embodiments, the condition, disease or disorder is selected from major adverse cardiovascular events such as cardiovascular death, non-fatal myocardial infarction and non-fatal stroke in patients with a prior heart attack and inflammatory atherosclerosis (see for example, NCT01327846).

In some embodiments, the condition, disease or disorder is selected from metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as Osteoarthritis, osteoporosis and osteopetrosis disorders eye disease, such as glaucoma and macular degeneration, diseased caused by viral infection such as HIV and AIDS, autoimmune disease such as Rheumatoid Arthritis, Systemic Lupus Erythematosus, Autoimmune Thyroiditis, Addison's disease, pernicious anemia, cancer and aging.

In some embodiments, the condition, disease or disorder is a cardiovascular indication. In some embodiments, the condition, disease or disorder is myocardial infraction. In some embodiments, the condition, disease or disorder is stroke.

In some embodiments, the condition, disease or disorder is obesity.

In some embodiments, the condition, disease or disorder is Type 2 Diabetes.

In some embodiments, the condition, disease or disorder is NASH.

In some embodiments, the condition, disease or disorder is Alzheimer's disease.

In some embodiments, the condition, disease or disorder is gout.

In some embodiments, the condition, disease or disorder is SLE.

In some embodiments, the condition, disease or disorder is rheumatoid arthritis.

In some embodiments, the condition, disease or disorder is IBD.

In some embodiments, the condition, disease or disorder is multiple sclerosis.

In some embodiments, the condition, disease or disorder is COPD.

In some embodiments, the condition, disease or disorder is asthma.

In some embodiments, the condition, disease or disorder is scleroderma.

In some embodiments, the condition, disease or disorder is pulmonary fibrosis.

In some embodiments, the condition, disease or disorder is age related macular degeneration (AMD).

In some embodiments, the condition, disease or disorder is cystic fibrosis.

In some embodiments, the condition, disease or disorder is Muckle Wells syndrome.

In some embodiments, the condition, disease or disorder is familial cold autoinflammatory syndrome (FCAS).

In some embodiments, the condition, disease or disorder is chronic neurologic cutaneous and articular syndrome.

In some embodiments, the condition, disease or disorder is selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; acute myeloid leukemia (AML) chronic myeloid leukemia (CIVIL); gastric cancer; and lung cancer metastasis.

In some embodiments, the condition, disease or disorder is selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; gastric cancer; and lung cancer metastasis.

In some embodiments, the condition, disease or disorder is MDS.

In some embodiments, the condition, disease or disorder is non-small lung cancer in patients carrying mutation or overexpression of NLRP3.

In some embodiments, the condition, disease or disorder is ALL in patients resistant to glucocorticoids treatment.

In some embodiments, the condition, disease or disorder is LCH.

In some embodiments, the condition, disease or disorder is multiple myeloma.

In some embodiments, the condition, disease or disorder is promyelocytic leukemia.

In some embodiments, the condition, disease or disorder is gastric cancer.

In some embodiments, the condition, disease or disorder is lung cancer metastasis.

In some embodiments of any of the methods described herein, the subject has or is suspected of having Crohn's disease, inflammatory bowel disease (IBD), or other gastrointestinal, autoimmune, or autoinflammatory disorders.

In some embodiments of any of the methods described herein, the subject has or is suspected of having hereditary periodic fever.

In some embodiments of any of the methods described herein, the subject has or is suspected of having Muckle-Wells syndrome (MWS).

In some embodiments of any of the methods described herein, the subject has or is suspected of having Cinca syndrome.

In some embodiments of any of the methods described herein, the subject has or is suspected of having deafness with or without inflammation.

In some embodiments of any of the methods described herein, the subject has or is suspected of having keratoendotheliitis fugax hereditaria.

In some embodiments of any of the methods described herein, the subject has or is suspected of having an inappropriate host response to infectious diseases where active infection exists at any body site. In some embodiments, the inappropriate host response to infectious disease where active infection exists at any body site is selected from the group consisting of: septic shock, disseminated intravascular coagulation, and adult respiratory distress syndrome.

In some embodiments of any of the methods described herein, the subject has or is suspected of having acute or chronic inflammation due to antigen, antibody, and/or complement deposition.

In some embodiments of any of the methods described herein, the subject has or is suspected of having an inflammatory disease or condition. In some embodiments, the inflammatory disease or condition is selected from the group consisting of: arthritis, cholangitis, colitis, encephalitis, endocarditis, glomerulonephritis, hepatitis, myocarditis, pancreatitis, pericarditis, reperfusion injury, vasculitis, osteoarthritis, COPD, periodontal disease, uveitis, cutaneous T-cell lymphoma, and mucositis such as oral mucositis, esophageal mucositis, and intestinal mucositis.

In some embodiments of any of the methods described herein, the inflammatory or autoimmune disorder is selected from the group consisting of: sickle cell disease, rheumatoid arthritis, juvenile arthritis, psoriatic arthritis, plaque psoriasis, ankylosing spondylitis, ulcerative colitis, Crohn's disease, inflammatory bowel disease, Behcet's disease, Takayasu's arteritis, atherosclerosis, gout, psoriasis, an infectious disease, asthma, peptic ulcer, periodontitis, dermatitis, diverticulitis, fibromyalgia, hepatitis, systemic lupus erythematosus, nephritis, appendicitis, bursitis, cystitis, encephalitis, gingivitis, meningitis, myelitis, neuritis, pharyngitis, phlebitis, prostatitis, rhinitis, sinusitis, tendonitis, testiculitis, tonsillitis, urethritis, vasculitis, vaginitis, Celiac disease, diverticulitis, glomerulonephritis, hidradenitis suppurativa, hypersensitivities, interstitial cystitis, Lichen planus, mast cell activation syndrome, mastocystosis, otitis, pelvic inflammatory disease, reperfusion injury, rheumatic fever, rhinitis, sarcoidosis, graft versus host disease, vasculitis, allergy, cancer, HIV, AIDS, scleroderma, Sjøgren's syndrome, anti-phospholipid antibody syndrome, myocarditis, postmyocardial infarction syndrome, postpericardiotomy syndrome, subacute bacterial endocarditis, anti-glomerular basement membrane nephritis, interstitial cystitis, lupus nephritis, autoimmune nephritis, autoimmune hepatitis, autoimmune hepatitis, primary biliary cholangitis, primary sclerosing cholangitis, primary schlerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, Lichen sclerosus, linear IgA disease, morphea, pemphigus vulgaris, pityriasis lichenoides et varioliforms acuta, Mucha-Habermann disease, psoriasis, systemic scleroderma, vitiligo, Addison's disease, autoimmune polyendocrine syndrome type 1, 2, or 3, autoimmune pancreatitis, diabetes mellitus type 1, autoimmune thyroiditis, Ord's thyroiditis, Graves' disease, autoimmune oophoritis, endometriosis, autoimmune orchitis, Sjogren's syndrome, autoimmune enteropathy, microscopic colitis, antiphospholipid syndrome, aplastic anemia, autoimmune hemolytic anemia, autoimmune lymphoproliferative syndrome, autoimmune neutropenia, autoimmune thrombocytopenic purpura, cold agglutinin disease, essential mixed cyroglobulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, Lyme disease, mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, sarcoidosis, Schnitzler syndrome, undifferentiated connective tissue disease, dermatomyositis, fibromyalgia, inclusion body myositis, myasthenia gravis, neuromyotonia, paraneoplastic cerebellar degeneration, polymyositis, acute disseminated encephalomyelitis, acute motor axonal neuropathy, anti-N-methyl-D-aspartate receptor encephalitis, Balo concentric sclerosis, Bickerstaff's encephalitis, chronic inflamatory demyelinating polyneuropathy, Guillain-Barre syndrome, Hashimoto's encephalopathy, idiopathic inflammatory demyelinating disease, Lambert-Eaton myasthenic syndrome, multiple sclerosis, Oshtoran syndrome, pediatric autoimmune neuropsychiatric disorder associated with Streptococcus, progressive inflammatory neuropathy, restless leg syndrome, Stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, Opsoclonus myoclonus syndrome, optic neuritis, scleritis, Susac's syndrome, sympathetic ophthalmia, Tolosa-Hunt syndrome, autoimmune inner ear disease, Meniere's disease, Behcet's disease, eosinophilic graunulomatosis with polyangiitis, giant cell arteritis, graunulomatosis with polyangiitis, IgA vasculitis, Kawasaki's disease, leukocytoclastic vasculitis, lupus vasculitis, microscopic polyangiitis, polyarteritis nodosa, polymyalgia rheumatica, urticarial vasculitis, vasculitis, primary immune deficiency, chronic fatigue syndrome, complex regional pain syndrome, eosinophilic esophagitis, gastritis, interstitial lung disease, POEMS syndrome, Raynaud's phenomenon, primary immunodeficiency, and pyoderma gangrenosum.

In some embodiments of any of the methods described herein, the subject has or is suspected of having acute and delayed hypersensitivity, graft rejection, or graft-versus-host disease (GVHD).

In some embodiments of any of the methods described herein, the subject has or is suspected of having an autoimmune disease selected from the group consisting of: Type 1 diabetes mellitus, rheumatoid arthritis, systemic lupus erythematosus, autoimmune thyroiditis, Addison's disease, pernicious anemia, multiple sclerosis, an inflammatory bowel disease (IBD), scleroderma, and psoriasis. In some embodiments, the IBD is selected from the group consisting of: Crohn's disease, ulcerative colitis, autoimmune colitis, iatrogenic autoimmune colitis, ulcerative colitis, colitis induced by one or more chemotherapeutic agents, colitis induced by treatment with adoptive cell therapy, colitis associated with one or more alloimmune diseases such as GVHD, radiation enteritis, collagenous colitis, lymphocytic colitis, microscopic colitis, and radiation enteritis, celiac disease, and inflammatory bowel syndrome.

In some embodiments of any of the methods described herein, the subject has or is suspected of having a metabolic disorder. In some embodiments, the metabolic disorder selected from the group consisting of type 2 diabetes, atherosclerosis, obesity, gout, and pseudogout.

In some embodiments of any of the methods described herein, the subject has or is suspected of having a disease of the central nervous system. In some embodiments, the disease of the central nervous system is selected from the group consisting of: Alzheimer's disease, multiple sclerosis, amyotrophic lateral sclerosis, and Parkinson's disease.

In some embodiments of any of the methods described herein, the subject has or is suspected of having a lung disease. In some embodiments, the lung disease is asthma, COPD, pulmonary idiopathic fibrosis, or cystic fibrosis.

In some embodiments of any of the methods described herein, the subject has or is suspected of having a liver disease. In some embodiments, the liver disease is selected from the group consisting of: NASH syndrome, viral hepatitis, and cirrhosis.

In some embodiments of any of the methods described herein, the subject has or is suspected of having a pancreatic disease, such as acute or chronic pancreatitis.

In some embodiments of any of the methods described herein, the subject has or is suspected of having kidney disease, such as acute or chronic kidney injury.

In some embodiments of any of the methods described herein, the subject has or is suspected of having an intestinal disease, such as Crohn's disease or ulcerative colitis.

In some embodiments of any of the methods described herein, the subject has or is suspected of having a skin disease, such as psoriasis.

In some embodiments of any of the methods described herein, the subject has or is suspected of having a musculoskeletal disease, such as scleroderma.

In some embodiments of any of the methods described herein, the subject has or is suspected of having a vessel disorder, such as giant cell arteritis.

In some embodiments of any of the methods described herein, the subject has a bone disorder, such as osteoarthritis, osteoporosis, and osteopetrosis disorders.

In some embodiments of any of the methods described herein, the subject has or is suspected of having an eye disease, such as glaucoma or macular degeneration, such as age-related macular degeneration.

In some embodiments of any of the methods described herein, the subject has or is suspected of having a disease caused by viral infection, such as HIV or AIDS.

In some embodiments of any of the methods described herein, the subject has or is suspected of having a cancer, such as non-small cell lung cancer, acute lymphoblastic leukemia (ALL) (ALL in patients resistant to glucocorticoid treatment), multiple myeloma, promyelocytic leukemia, gastric cancer, and lung cancer metastasis.

In some embodiments of any of the methods described herein, the subject has or is suspected of having a cardiovascular disease. In some embodiments, the cardiovascular disease is myocardial infarction, stroke, or heart failure.

In some embodiments of any of the methods described herein, the subject has or is suspected of having Crohn's disease, inflammatory bowel disease (IBD), or other gastrointestinal, autoimmune, or autoinflammatory disorders.

In some embodiments of any of the methods described herein, the subject has or is suspected of having hereditary periodic fever.

In some embodiments of any of the methods described herein, the subject has or is suspected of having Muckle-Wells syndrome (MWS).

In some embodiments of any of the methods described herein, the subject has or is suspected of having Cinca syndrome.

In some embodiments of any of the methods described herein, the subject has or is suspected of having deafness with or without inflammation.

In some embodiments of any of the methods described herein, the subject has or is suspected of having keratoendotheliitis fugax hereditaria.

In some embodiments of any of the methods described herein, the subject has or is suspected of having an inappropriate host response to infectious diseases where active infection exists at any body site. In some embodiments, the inappropriate host response to infectious disease where active infection exists at any body site is selected from the group consisting of: septic shock, disseminated intravascular coagulation, and adult respiratory distress syndrome.

In some embodiments of any of the methods described herein, the subject has or is suspected of having acute or chronic inflammation due to antigen, antibody, and/or complement deposition.

In some embodiments of any of the methods described herein, the subject has or is suspected of having an inflammatory disease or condition. In some embodiments, the inflammatory disease or condition is selected from the group consisting of: arthritis, cholangitis, colitis, encephalitis, endocarditis, glomerulonephritis, hepatitis, myocarditis, pancreatitis, pericarditis, reperfusion injury, vasculitis, osteoarthritis, COPD, periodontal disease, uveitis, cutaneous T-cell lymphoma, and mucositis such as oral mucositis, esophageal mucositis, and intestinal mucositis.

In some embodiments of any of the methods described herein, the subject has or is suspected of having acute and delayed hypersensitivity, graft rejection, or graft-versus-host disease (GVHD).

In some embodiments of any of the methods described herein, the subject has or is suspected of having an autoimmune disease selected from the group consisting of: Type 1 diabetes mellitus, rheumatoid arthritis, systemic lupus erythematosus, autoimmune thyroiditis, Addison's disease, pernicious anemia, multiple sclerosis, an inflammatory bowel disease (IBD), scleroderma, and psoriasis. In some embodiments, the IBD is selected from the group consisting of: Crohn's disease, ulcerative colitis, autoimmune colitis, iatrogenic autoimmune colitis, ulcerative colitis, colitis induced by one or more chemotherapeutic agents, colitis induced by treatment with adoptive cell therapy, colitis associated with one or more alloimmune diseases such as GVHD, radiation enteritis, collagenous colitis, lymphocytic colitis, microscopic colitis, and radiation enteritis, celiac disease, and inflammatory bowel syndrome.

In some embodiments of any of the methods described herein, the subject has or is suspected of having a metabolic disorder. In some embodiments, the metabolic disorder selected from the group consisting of type 2 diabetes, atherosclerosis, obesity, gout, and pseudogout.

In some embodiments of any of the methods described herein, the subject has or is suspected of having a disease of the central nervous system. In some embodiments, the disease of the central nervous system is selected from the group consisting of: Alzheimer's disease, multiple sclerosis, amyotrophic lateral sclerosis, and Parkinson's disease.

In some embodiments of any of the methods described herein, the subject has or is suspected of having a lung disease. In some embodiments, the lung disease is asthma, COPD, pulmonary idiopathic fibrosis, or cystic fibrosis.

In some embodiments of any of the methods described herein, the subject has or is suspected of having a liver disease. In some embodiments, the liver disease is selected from the group consisting of: NASH syndrome, viral hepatitis, and cirrhosis.

In some embodiments of any of the methods described herein, the subject has or is suspected of having a pancreatic disease, such as acute or chronic pancreatitis.

In some embodiments of any of the methods described herein, the subject has or is suspected of having kidney disease, such as acute or chronic kidney injury.

In some embodiments of any of the methods described herein, the subject has or is suspected of having an intestinal disease, such as Crohn's disease or ulcerative colitis.

In some embodiments of any of the methods described herein, the subject has or is suspected of having a skin disease, such as psoriasis.

In some embodiments of any of the methods described herein, the subject has or is suspected of having a musculoskeletal disease, such as scleroderma.

In some embodiments of any of the methods described herein, the subject has or is suspected of having a vessel disorder, such as giant cell arteritis.

In some embodiments of any of the methods described herein, the subject has a bone disorder, such as osteoarthritis, osteoporosis, and osteopetrosis disorders.

In some embodiments of any of the methods described herein, the subject has or is suspected of having an eye disease, such as glaucoma or macular degeneration, such as age-related macular degeneration.

In some embodiments of any of the methods described herein, the subject has or is suspected of having a disease caused by viral infection, such as HIV or AIDS.

In some embodiments of any of the methods described herein, the subject has or is suspected of having a cancer, such as non-small cell lung cancer, acute lymphoblastic leukemia (ALL) (ALL in patients resistant to glucocorticoid treatment), multiple myeloma, promyelocytic leukemia, gastric cancer, and lung cancer metastasis.

In some embodiments of any of the methods described herein, the subject has or is suspected of having a cardiovascular disease. In some embodiments, the cardiovascular disease is myocardial infarction, stroke, or heart failure.

In some embodiments of any of the methods described herein, the subject has or is suspected of having: hereditary periodic fever, familial cold autoinflammatory syndrome (FCAS), Muckle-Wells syndrome, myelodysplastic syndrome (MDS), Langerhan's cell histiocytosis (LCH), neonatal onset multisystem inflammatory disease, Cinca syndrome, deafness with inflammation, deafness without inflammation, keratoendotheliitis fugax hereditaria, silicosis, asbestosis, or chronic neurologic cutaneous and articular syndrome.

In some embodiments of any of the methods described herein, the subject has been exposed to, or is suspected of having been exposed to, a toxic agent selected from the group consisting of: exogenous microbial stimuli, lipopolysaccharide (LPS), lipooligosaccharide, muramyl dipeptide (MDP), nigericin, maitotoxin, asbestos, and silica.

### Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the NLRP3 antagonist (e.g., any of the NLRP3 antagonists described herein or known in the art).

In certain embodiments, the second therapeutic agent or regimen is administered to the subject prior to contacting with or administering the NLRP3 antagonist (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the NLRP3 antagonist. By way of example, the second therapeutic agent or regimen and the NLRP3 antagonist are provided to the subject simultaneously in the same dosage form. As another example, the second therapeutic agent or regimen and the NLRP3 antagonist are provided to the subject concurrently in separate dosage forms.

In still other embodiments, the second therapeutic agent or regimen is administered to the subject after contacting with or administering the NLRP3 antagonist (e.g., about one hour after, or about 6 hours after, or about 12 hours after, or about 24 hours after, or about 48 hours after, or about 1 week after, or about 1 month after).

### Patient Selection

In some embodiments, the methods described herein include the step of identifying a subject (e.g., a patient) in need of treatment for an indication related to NLRP3 activity, such as an indication related to NLRP3 polymorphism.

In some embodiments, the methods described herein further include the step of identifying a subject (e.g., a patient) in need of treatment for an indication related to NLRP3 activity, such as an indication related to NLRP3 where a polymorphism in a NLRP3 gene is a gain-of-function mutation (e.g., a NLRP3 protein having a Q705K amino acid substitution, a T350M amino acid substitution, a R262M amino acid substitution, a A441V amino acid substitution, a V200M amino acid substitution, an E629G amino acid substitution, a L355P amino acid substitution, a R260W amino acid substitution, a G571R amino acid substitution, a A354V amino acid substitution, a D305N amino acid substitution, a F311S amino acid substitution, a R920Q amino acid substitution, or a D21H amino acid substitution, each numbered according to the mature NLRP3 protein sequence of SEQ ID NO: 1).

In some embodiments, the methods described herein include the step of identifying a subject (e.g., a patient) in need of treatment for an indication related to NLRP3 activity, such as an indication related to NLRP3 polymorphism found in CAPS syndromes.

In some embodiments, the methods described herein include the step of identifying a subject (e.g., a patient) in need of treatment for an indication related to NLRP3 activity, such as an indication related NLRP3 polymorphism where the polymorphism is VAR 014104 (R262W, numbered according to the mature NLRP3 protein sequence of SEQ ID NO: 1).

In some embodiments, the methods described herein include the step of identifying a subject (e.g., a patient) in need of treatment for an indication related to NLRP3 activity, such as an indication related NLRP3 polymorphism where the polymorphism is a natural variant reported in www.uniprot.org/uniprot/Q96P20.

In some embodiments, the methods described herein further include the step of identifying a subject (e.g., a patient) in need of treatment for an indication related to NLRP3 activity, such as an indication related to a point mutation in a gene involved in NLRP3 signaling.

Methods of Detecting the Level of NLRP3 Inflammasome Activity and/or Expression

In some embodiments of any of the methods described herein, the NLRP3 inflammmasome activity is the secretion of IL-18. In some embodiments of any of the methods described herein, the NLPR3 inflammasome activity is the secretion of IL-1β. In some embodiments of any of the methods described herein, the NLRP3 inflammasome activity is caspase-1 activity in a mammalian cell (e.g., a mammalian cell obtained from the subject). Non-limiting examples of methods that can be used to detect the secretion of IL-18 and IL-1β include immunohistochemistry, immunoassays, e.g., enzyme-linked immunosorbent assay (ELISA), sandwich ELISA, immunoprecipitation, and immunofluorescent assay. Non-limiting examples of commercially available assays for determining caspase-1 activity include Caspase 1 Assay Kit (Fluormetric) (ab39412) (Abcam), FAM-FLICA® Caspase-1 Assay Kit (ImmunoChemistry), Caspase-1 Colorimetric Assay Kit (K111) (Biovision, Inc.), and Caspase-1/ICE Colorimetric Assay Kit (R&D Systems).

In some embodiments of any of the methods described herein, the NLRP3 inflammasome activity can be the level of expression of an upstream activator of NLRP3 inflammasomes (e.g., the level of one or more (e.g., two, three, four, five, or six) of lipocalin-2 protein, lipocalin-2 mRNA, S100A8 protein, S1008A8 mRNA, S100A9 protein, or S100A9 mRNA) in a mammalian cell (e.g., a mammalian cell obtained from a subject). Non-limiting assays that can be used to determine NLRP3 activity include: Southern blot analysis, Norther blot analysis, polymerase chain reaction (PCR)-based methods, e.g., next generation sequencing, reverse transcription polymerase chain reaction (RT-PCR), TaqMan™, microarray analysis, immunohistochemistry, immunoassays, e.g., enzyme-linked immunosorbent assay (ELISA), sandwich ELISA, immunoprecipitation, immunofluorescent assay, mass spectrometry, immunoblot (Western blot), RIA, and flow cytometry. In some embodiments of any of the methods described herein, a mammalian cell have an increased level of NLRP3 activity can be identified by detecting the presence of one of more of the following the mammalian cell: a gain-of-function mutation in a NLRP3 gene (e.g., a NLRP3 protein having a Q705K amino acid substitution, a T350M amino acid substitution, a R262M amino acid substitution, a A441V amino acid substitution, a V200M amino acid substitution, an E629G amino acid substitution, a L355P amino acid substitution, a R260W amino acid substitution, a G571R amino acid substitution, a A354V amino acid substitution, a D305N amino acid substitution, a F311S amino acid substitution, a R920Q amino acid substitution, or a D21H amino acid substitution, each numbered according to the mature NLRP3 protein sequence of SEQ ID NO: 1); a loss-of-function mutations in one or more of a CARD8 gene (e.g., a C allele at rs2043211); a T allele at rs3024505 flanking IL10 gene; detection of a R620W amino acid substitution in PTPN22; detection of a C allele at rs478582 in the PTPN2 gene; detection of a G allele at rs713875 in the MTMR3 gene; detection of an C allele at rs1042058 in the TPL2 gene; and detection of a ATG16L1 gene that encodes a ATG16L1 protein having a T300A amino acid substitution. Non-limiting examples of assays that can be used to determine the level of the presence of any of these mutations (e.g., any of the mutations described herein) include Southern blot analysis, Norther blot analysis, polymerase chain reaction (PCR)-based methods, e.g., next generation sequencing, reverse transcription polymerase chain reaction (RT-PCR), TaqMan™, and microarray analysis.

In some embodiments of any of the methods described herein, the NLRP3 inflammasome expression can be determined by detecting the level of one or more (e.g., two, three, four, five, six, or seven) of: NLRP3 protein, ASC protein, procaspase-1 protein, caspase-1 protein, NLRP3 mRNA, ASC mRNA, and procaspase-1 mRNA, in a mammalian cell (e.g., in a mammalian cell obtained from the subject). Non-limiting examples of assays that can be used to determine the level of NLRP3 protein, ASC protein, procaspase-1 protein, and caspase-1 protein include immunohistochemistry, immunoassays, e.g., enzyme-linked immunosorbent assay (ELISA), sandwich ELISA, immunoprecipitation, immunofluorescent assay, and flow cytometry. Non-limiting examples of assays that can be used to determine the level of NLRP3 mRNA, ASC mRNA, and procaspase-1 mRNA include Southern blot analysis, Norther blot analysis, polymerase chain reaction (PCR)-based methods, e.g., next generation sequencing, reverse transcription polymerase chain reaction (RT-PCR), TaqMan™, and microarray analysis.

In some embodiments of any of the methods described herein, the NLRP3 inflammasome expression can be determined by detecting the level of one or more of CRP protein, SAA protein, HP protein, ceruloplasmin protein, IL-6 protein (e.g., mature or pro-IL-6 protein), calprotectin (S100A8) protein, IL-8 protein (e.g., mature or pro-IL-8 protein), leukotriene B4 protein, myeloperoxidase protein, CRP mRNA, SAA mRNA, HP mRNA, ceruloplasmin mRNA, pro-IL-6 mRNA, calprotectin (S100A8) mRNA, pro-IL-8 mRNA, leukotriene B4 mRNA, and myeloperoxidase mRNA. Non-limiting examples of assays that can be used to determine the level of CRP protein, SAA protein, HP protein, ceruloplasmin protein, IL-6 protein (e.g., mature or pro-IL-6 protein), calprotectin (S100A8) protein, IL-8 protein (e.g., mature or pro-IL-8 protein), leukotriene B4 protein, myeloperoxidase protein include immunohistochemistry, immunoassays, e.g., enzyme-linked immunosorbent assay (ELISA), sandwich ELISA, immunoprecipitation, immunofluorescent assay, and flow cytometry. Non-limiting examples of assays that can be used to determine the level of CRP mRNA, SAA mRNA, HP mRNA, ceruloplasmin mRNA, pro-IL-6 mRNA, calprotectin (S100A8) mRNA, pro-IL-8 mRNA, leukotriene B4 mRNA, and myeloperoxidase mRNA include Southern blot analysis, Norther blot analysis, polymerase chain reaction (PCR)-based methods, e.g., next generation sequencing, reverse transcription polymerase chain reaction (RT-PCR), TaqMan™, and microarray analysis.

In some embodiments of any of the methods described herein, the level of the protein or mRNA can be detected in a biological sample including blood, serum, exosomes, plasma, tissue, urine, feces, sputum, and cerebrospinal fluid.

In some embodiments of any of the methods described herein, the level of at least one (e.g., 2, 3, 4, 5, 6, 7 or 8) NLRP3 inflammasome activity and/or expression can be determined, e.g., in any combination.

In one aspect, the cell can be a cell isolated from a subject who has been screened for the presence of an inflammatory disease or indication that is associated with a mutation in a NLRP3 activity.

### Reference Levels

In some embodiments of any of the methods described herein, the reference can be a corresponding level detected in a similar cell or sample obtained from a healthy subject (e.g., a subject that has not been diagnosed or identified as having an inflammatory disease or autoimmune disorder, or any disorder associated with aberrant NLRP3 inflammasome activity and/or expression) (e.g., a subject who is not suspected or is not at increased risk of developing an inflammatory disease or autoimmune disease, or any disorder associated with aberrant NLRP3 inflammasome activity and/or expression) (e.g., a subject that does not present with any symptom of an inflammatory disease or autoimmune disease, or any disorder associated with aberrant NLRP3 inflammasome activity and/or expression).

In some embodiments, a reference level can be a percentile value (e.g., mean value, 99% percentile, 95% percentile, 90% percentile, 85% percentile, 80% percentile, 75% percentile, 70% percentile, 65% percentile, 60% percentile, 55% percentile, or 50% percentile) of the corresponding levels detected in similar samples in a population of healthy subjects (e.g., a population of subjects that have not been diagnosed or identified as having an inflammatory disease or autoimmune disorder, or any disorder associated with aberrant NLRP3 inflammasome activity and/or expression) (e.g., a population of subjects who are not suspected or are not at increased risk of developing an inflammatory disease or autoimmune disease, or any disorder associated with aberrant NLRP3 inflammasome activity and/or expression) (e.g., a population of subjects that do not present with any symptom of an inflammatory disease or autoimmune disease, or any disorder associated with aberrant NLRP3 inflammasome activity and/or expression).

In some embodiments, a reference can be a corresponding level detected in a similar sample obtained from the subject at an earlier time point.

### NLR Family Pyrin Domain Containing 3 (NLRP3) Antagonists

In any of the methods described herein, the NLRP3 antagonist can be any of the NLRP3 antagonists described herein (e.g., any of the compounds described in this section). In any of the methods described herein, the NLRP3 antagonist has an IC50 of between about 1 nM and about 10 μM (e.g., between about 1 nM and about 9 μM, between about 1 nM and about 8 μM, between about 1 nM and about 7 μM, between about 1 nM and about 6 μM, between about 1 nM and about 5 μM, between about 1 nM and about 4 μM, between about 1 nM and about 3 μM, between about 1 nM and about 2 μM, between about 1 nM and about 1 μM, between about 1 nM and about 950 nM, between about 1 nM and about 900 nM, between about 1 nM and about 850 nM, between about 1 nM and about 800 nM, between about 1 nM and about 750 nM, between about 1 nM and about 700 nM, between about 1 nM and about 650 nM, between about 1 nM and about 600 nM, between about 1 nM and about 550 nM, between about 1 nM and about 500 nM, between about 1 nM and about 450 nM, between about 1 nM and about 400 nM, between about 1 nM and about 350 nM, between about 1 nM and about 300 nM, between about 1 nM and about 250 nM, between about 1 nM and about 200 nM, between about 1 nM and about 150 nM, between about 1 nM and about 100 nM, between about 1 nM and about 95 nM, between about 1 nM and about 90 nM, between about 1 nM and about 85 nM, between about 1 nM and about 80 nM, between about 1 nM and about 75 nM, between about 1 nM and about 70 nM, between about 1 nM and about 65 nM, between about 1 nM and about 60 nM, between about 1 nM and about 55 nM, between about 1 nM and about 50 nM, between about 1 nM and about 45 nM, between about 1 nM and about 40 nM, between about 1 nM and about 35 nM, between about 1 nM and about 30 nM, between about 1 nM and about 25 nM, between about 1 nM and about 20 nM, between about 1 nM and about 15 nM, between about 1 nM and about 10 nM, between about 1 nM and about 5 nM, between about 1 nM and about 4 nM, between about 1 nM and about 3 nM, between about 1 nM and about 2 nM, between about 2 nM and about 10 μM, between about 2 nM and about 9 μM, between about 2 nM and about 8 μM, between about 2 nM and about 7 μM, between about 2 nM and about 6 μM, between about 2 nM and about 5 μM, between about 2 nM and about 4 μM, between about 2 nM and about 3 μM, between about 2 nM and about 2 μM, between about 2 nM and about 1 μM, between about 2 nM and about 950 nM, between about 2 nM and about 900 nM, between about 2 nM and about 850 nM, between about 2 nM and about 800 nM, between about 2 nM and about 750 nM, between about 2 nM and about 700 nM, between about 2 nM and about 650 nM, between about 2 nM and about 600 nM, between about 2 nM and about 550 nM, between about 2 nM and about 500 nM, between about 2 nM and about 450 nM, between about 2 nM and about 400 nM, between about 2 nM and about 350 nM, between about 2 nM and about 300 nM, between about 2 nM and about 250 nM, between about 2 nM and about 200 nM, between about 2 nM and about 150 nM, between about 2 nM and about 100 nM, between about 2 nM and about 95 nM, between about 2 nM and about 90 nM, between about 2 nM and about 85 nM, between about 2 nM and about 80 nM, between about 2 nM and about 75 nM, between about 2 nM and about 70 nM, between about 2 nM and about 65 nM, between about 2 nM and about 60 nM, between about 2 nM and about 55 nM, between about 2 nM and about 50 nM, between about 2 nM and about 45 nM, between about 2 nM and about 40 nM, between about 2 nM and about 35 nM, between about 2 nM and about 30 nM, between about 2 nM and about 25 nM, between about 2 nM and about 20 nM, between about 2 nM and about 15 nM, between about 2 nM and about 10 nM, between about 2 nM and about 5 nM, between about 2 nM and about 4 nM, between about 2 nM and about 3 nM, between about 5 nM and about 10 μM, between about 5 nM and about 9 μM, between about 5 nM and about 8 μM, between about 5 nM and about 7 μM, between about 5 nM and about 6 μM, between about 5 nM and about 5 μM, between about 5 nM and about 4 μM, between about 5 nM and about 3 μM, between about 5 nM and about 2 μM, between about 5 nM and about 1 μM, between about 5 nM and about 950 nM, between about 5 nM and about 900 nM, between about 5 nM and about 850 nM, between about 5 nM and about 800 nM, between about 5 nM and about 750 nM, between about 5 nM and about 700 nM, between about 5 nM and about 650 nM, between about 5 nM and about 600 nM, between about 5 nM and about 550 nM, between about 5 nM and about 500 nM, between about 5 nM and about 450 nM, between about 5 nM and about 400 nM, between about 5 nM and about 350 nM, between about 5 nM and about 300 nM, between about 5 nM and about 250 nM, between about 5 nM and about 200 nM, between about 5 nM and about 150 nM, between about 5 nM and about 100 nM, between about 5 nM and about 95 nM, between about 5 nM and about 90 nM, between about 5 nM and about 85 nM, between about 5 nM and about 80 nM, between about 5 nM and about 75 nM, between about 5 nM and about 70 nM, between about 5 nM and about 65 nM, between about 5 nM and about 60 nM, between about 5 nM and about 55 nM, between about 5 nM and about 50 nM, between about 5 nM and about 45 nM, between about 5 nM and about 40 nM, between about 5 nM and about 35 nM, between about 5 nM and about 30 nM, between about 5 nM and about 25 nM, between about 5 nM and about 20 nM, between about 5 nM and about 15 nM, between about 5 nM and about 10 nM, between about 10 nM and about 10 μM, between about 10 nM and about 9 μM, between about 10 nM and about 8 μM, between about 10 nM and about 7 μM, between about 10 nM and about 6 μM, between about 10 nM and about 5 μM, between about 10 nM and about 4 μM, between about 10 nM and about 3 μM, between about 10 nM and about 2 μM, between about 10 nM and about 1 μM, between about 10 nM and about 950 nM, between about 10 nM and about 900 nM, between about 10 nM and about 850 nM, between about 10 nM and about 800 nM, between about 10 nM and about 750 nM, between about 10 nM and about 700 nM, between about 10 nM and about 650 nM, between about 10 nM and about 600 nM, between about 10 nM and about 550 nM, between about 10 nM and about 500 nM, between about 10 nM and about 450 nM, between about 10 nM and about 400 nM, between about 10 nM and about 350 nM, between about 10 nM and about 300 nM, between about 10 nM and about 250 nM, between about 10 nM and about 200 nM, between about 10 nM and about 150 nM, between about 10 nM and about 100 nM, between about 10 nM and about 95 nM, between about 10 nM and about 90 nM, between about 10 nM and about 85 nM, between about 10 nM and about 80 nM, between about 10 nM and about 75 nM, between about 10 nM and about 70 nM, between about 10 nM and about 65 nM, between about 10 nM and about 60 nM, between about 10 nM and about 55 nM, between about 10 nM and about 50 nM, between about 10 nM and about 45 nM, between about 10 nM and about 40 nM, between about 10 nM and about 35 nM, between about 10 nM and about 30 nM, between about 10 nM and about 25 nM, between about 10 nM and about 20 nM, between about 10 nM and about 15 nM, between about 50 nM and about 10 μM, between about 50 nM and about 9 μM, between about 50 nM and about 8 μM, between about 50 nM and about 7 μM, between about 50 nM and about 6 μM, between about 50 nM and about 5 μM, between about 50 nM and about 4 μM, between about 50 nM and about 3 μM, between about 50 nM and about 2 μM, between about 50 nM and about 1 μM, between about 50 nM and about 950 nM, between about 50 nM and about 900 nM, between about 50 nM and about 850 nM, between about 50 nM and about 800 nM, between about 50 nM and about 750 nM, between about 50 nM and about 700 nM, between about 50 nM and about 650 nM, between about 50 nM and about 600 nM, between about 50 nM and about 550 nM, between about 50 nM and about 500 nM, between about 50 nM and about 450 nM, between about 50 nM and about 400 nM, between about 50 nM and about 350 nM, between about 50 nM and about 300 nM, between about 50 nM and about 250 nM, between about 50 nM and about 200 nM, between about 50 nM and about 150 nM, between about 50 nM and about 100 nM, between about 50 nM and about 95 nM, between about 50 nM and about 90 nM, between about 50 nM and about 85 nM, between about 50 nM and about 80 nM, between about 50 nM and about 75 nM, between about 50 nM and about 70 nM, between about 50 nM and about 65 nM, between about 50 nM and about 60 nM, between about 50 nM and about 55 nM, between about 100 nM and about 10 μM, between about 100 nM and about 9 μM, between about 100 nM and about 8 μM, between about 100 nM and about 7 μM, between about 100 nM and about 6 μM, between about 100 nM and about 5 μM, between about 100 nM and about 4 μM, between about 100 nM and about 3 μM, between about 100 nM and about 2 μM, between about 100 nM and about 1 μM, between about 100 nM and about 950 nM, between about 100 nM and about 900 nM, between about 100 nM and about 850 nM, between about 100 nM and about 800 nM, between about 100 nM and about 750 nM, between about 100 nM and about 700 nM, between about 100 nM and about 650 nM, between about 100 nM and about 600 nM, between about 100 nM and about 550 nM, between about 100 nM and about 500 nM, between about 100 nM and about 450 nM, between about 100 nM and about 400 nM, between about 100 nM and about 350 nM, between about 100 nM and about 300 nM, between about 100 nM and about 250 nM, between about 100 nM and about 200 nM, between about 100 nM and about 150 nM, between about 200 nM and about 10 μM, between about 200 nM and about 9 μM, between about 200 nM and about 8 μM, between about 200 nM and about 7 μM, between about 200 nM and about 6 μM, between about 200 nM and about 5 μM, between about 200 nM and about 4 μM, between about 200 nM and about 3 μM, between about 200 nM and about 2 μM, between about 200 nM and about 1 μM, between about 200 nM and about 950 nM, between about 200 nM and about 900 nM, between about 200 nM and about 850 nM, between about 200 nM and about 800 nM, between about 200 nM and about 750 nM, between about 200 nM and about 700 nM, between about 200 nM and about 650 nM, between about 200 nM and about 600 nM, between about 200 nM and about 550 nM, between about 200 nM and about 500 nM, between about 200 nM and about 450 nM, between about 200 nM and about 400 nM, between about 200 nM and about 350 nM, between about 200 nM and about 300 nM, between about 200 nM and about 250 nM, between about 250 nM and about 10 μM, between about 250 nM and about 9 μM, between about 250 nM and about 8 μM, between about 250 nM and about 7 μM, between about 250 nM and about 6 μM, between about 250 nM and about 5 μM, between about 250 nM and about 4 μM, between about 250 nM and about 3 μM, between about 250 nM and about 2 μM, between about 250 nM and about 1 μM, between about 250 nM and about 950 nM, between about 250 nM and about 900 nM, between about 250 nM and about 850 nM, between about 250 nM and about 800 nM, between about 250 nM and about 750 nM, between about 250 nM and about 700 nM, between about 250 nM and about 650 nM, between about 250 nM and about 600 nM, between about 250 nM and about 550 nM, between about 250 nM and about 500 nM, between about 250 nM and about 450 nM, between about 250 nM and about 400 nM, between about 250 nM and about 350 nM, between about 250 nM and about 300 nM, between about 500 nM and about 10 μM, between about 500 nM and about 9 μM, between about 500 nM and about 8 μM, between about 500 nM and about 7 μM, between about 500 nM and about 6 μM, between about 500 nM and about 5 μM, between about 500 nM and about 4 μM, between about 500 nM and about 3 μM, between about 500 nM and about 2 μM, between about 500 nM and about 1 μM, between about 500 nM and about 950 nM, between about 500 nM and about 900 nM, between about 500 nM and about 850 nM, between about 500 nM and about 800 nM, between about 500 nM and about 750 nM, between about 500 nM and about 700 nM, between about 500 nM and about 650 nM, between about 500 nM and about 600 nM, between about 500 nM and about 550 nM, between about 750 nM and about 10 μM, between about 750 nM and about 9 μM, between about 750 nM and about 8 μM, between about 750 nM and about 7 μM, between about 750 nM and about 6 μM, between about 750 nM and about 5 μM, between about 750 nM and about 4 μM, between about 750 nM and about 3 μM, between about 750 nM and about 2 μM, between about 750 nM and about 1 μM, between about 750 nM and about 950 nM, between about 750 nM and about 900 nM, between about 750 nM and about 850 nM, between about 750 nM and about 800 nM, between about 950 nM and about 10 μM, between about 950 nM and about 9 μM, between about 950 nM and about 8 μM, between about 950 nM and about 7 μM, between about 950 nM and about 6 μM, between about 950 nM and about 5 μM, between about 950 nM and about 4 μM, between about 950 nM and about 3 μM, between about 950 nM and about 2 μM, between about 950 nM and about 1 μM, between about 1 μM and about 10 μM, between about 1 μM and about 9 μM, between about 1 μM and about 8 μM, between about 1 μM and about 7 μM, between about 1 μM and about 6 μM, between about 1 μM and about 5 μM, between about 1 μM and about 4 μM, between about 1 μM and about 3 μM, between about 1 μM and about 2 μM, between about 2 μM and about 10 μM, between about 2 μM and about 9 μM, between bout 2 μM and about 8 μM, between about 2 μM and about 7 μM, between about 2 μM and about 6 μM, between about 2 μM and about 5 μM, between about 2 μM and about 4 μM, between about 2 μM and about 3 μM, between about 4 μM and about 10 μM, between about 4 μM and about 9 μM, between about 4 μM and about 8 μM, between about 4 μM and about 7 μM, between about 4 μM and about 6 μM, between about 4 μM and about 5 μM, between about 5 μM and about 10 μM, between about 5 μM and about 9 μM, between about 5 μM and about 8 μM, between about 5 μM and about 7 μM, between about 5 μM and about 6 μM, between about 6 μM and about 10 μM, between about 6 μM and about 9 μM, between about 6 μM and about 8 μM, between about 6 μM and about 7 μM; between about 7 μM and about 10 μM, between about 7 μM and about 9 μM, between about 7 μM and about 8 μM, between about 8 μM and about 10 μM, between about 8 μM and about 9 μM, or between about 9 μM and about 10 μM) for NLRP3.

In one aspect, an NLRP3 antagonist is a compound of Formula I,

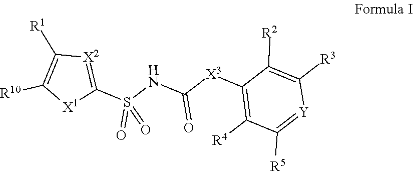

or a pharmaceutically acceptable salt thereof, wherein:

X¹ is O, S, or NH;

X² is N or CR⁹;

X³ is NH or O;

or when X³ is NH, X³ and R² taken together with the atoms connecting them form a four-to-seven-membered heterocyclic ring optionally substituted with one or more R¹⁶; or when X³ is NH, X³ and R⁴ taken together with the atoms connecting them form a four-to-seven-membered heterocyclic ring optionally substituted with one or more R¹⁶; Y is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R⁹ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R³ is hydrogen, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

provided that at least one of R², R³, R⁴ and R⁵ is not hydrogen, and that R² and R⁴ are not both hydroxymethyl;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B, wherein ring A is

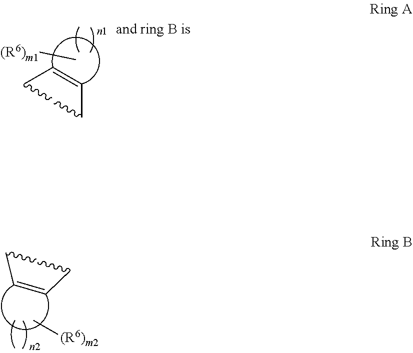

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from H, C₁-C₆ alkyl, C₃-C₆ cycloalkyl and C₃-C₆ heterocycloalkyl; wherein R¹ is optionally substituted with one or more substituents each independently, selected from hydroxy, oxo, C₁-C₆ alkoxy, NR¹¹R¹², ═NR¹³, COOC₁-C₆ alkyl, and CONR¹¹R¹²;
R¹⁰ is selected from H, C₁-C₆ alkyl, C₃-C₆ cycloalkyl and C₃-C₆ heterocycloalkyl; wherein R¹⁰ is optionally substituted with one or more substituents each independently, selected from hydroxy, oxo, C₁-C₆ alkoxy, NR¹¹R¹², ═NR¹³, COOC₁-C₆ alkyl, and CONR¹¹R¹²;
or R¹ and R¹⁰ taken together with the atoms connecting them form a 3-to-8-membered carbocyclic or heterocyclic ring containing 1 or 2 heteroatoms independently selected from O, N, and S, wherein the ring is optionally substituted with one or more substituents each independently selected from hydroxy, oxo, C₁-C₆ alkoxy, NR¹¹R¹², ═NR¹³, COOC₁-C₆ alkyl, and CONR¹¹R¹²;

R¹³ is C₁-C₆ alkyl;
each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;
each of R¹⁷ and R¹⁸ at each occurrence is independently selected from hydrogen and C₁-C₆ alkyl;
each 10⁶ is the same or different and is selected from H, C₁-C₆ alkyl, C₁-C₆ alkoxy, NR¹¹R¹², oxo, and ═NR¹³.

In some embodiments, NLRP3 antagonist is a compound of Formula I, provided that if the compound of formula I comprises ring A and ring B; X³ is NH; X² is CH; and R¹⁰ is H,

then (i) if X¹ is O and R¹ is C₁-C₆ alkyl optionally substituted with hydroxy, C₁-C₆ alkoxy, NR¹¹R¹², ═NR¹³, COOC₁-C₆ alkyl, or CONR¹¹R¹², then Y is not N, CF, CCl or CH, (ii) if X¹ is O and R¹ is C₁-C₆ alkyl substituted with oxo, then Y is not CH, and (iii) if X¹ is S, then Y is not CH;

and provided that if R² and R⁴ are each isopropyl; X³ is NH; X² is CH; R¹⁰ is H; and R¹ is C₁-C₆ alkyl optionally substituted with hydroxy, C₁-C₆ alkoxy, NR¹¹R¹², —NR₁₃, COOC₁-C₆ alkyl, or CONR¹¹R¹², then Y is not CH or CCl.

In another aspect, an NLRP3 antagonist is a compound of Formula I,

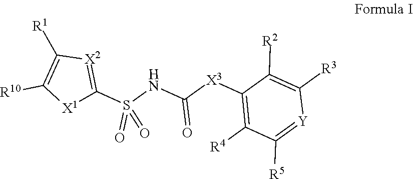

or a pharmaceutically acceptable salt thereof, wherein:

X¹ is O, S, or NH;

X² is N or CR⁹;

X³ is NH or O;

or when X³ is NH, X³ and R² taken together with the atoms connecting them form a four-to-seven-membered heterocyclic ring optionally substituted with one or more R¹⁶; or when X³ is NH, X³ and R⁴ taken together with the atoms connecting them form a four-to-seven-membered heterocyclic ring optionally substituted with one or more R¹⁶; Y is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R⁹ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R³ is hydrogen, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

provided that at least one of R², R³, R⁴ and R⁵ is not hydrogen, and that R² and R⁴ are not both hydroxymethyl;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

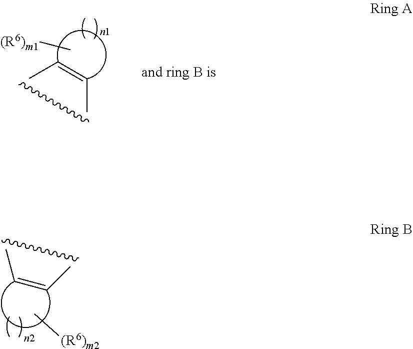

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from H, C₁-C₆ alkyl, C₃-C₆ cycloalkyl and C₃-C₆ heterocycloalkyl; wherein R¹ is optionally substituted with one or more substituents each independently, selected from hydroxy, oxo, C₁-C₆ alkoxy, NR¹¹R¹², ═NR¹³, COOC₁-C₆ alkyl, and CONR¹¹R¹²;

R¹⁰ is selected from H, C₁-C₆ alkyl, C₃-C₆ cycloalkyl and C₃-C₆ heterocycloalkyl; wherein R¹⁰ is optionally substituted with one or more substituents each, independently selected from hydroxy, oxo, C₁-C₆ alkoxy, NR¹¹R¹², ═NR¹³, COOC₁-C₆ alkyl, and CONR¹¹R¹²;

or R¹ and R¹⁰ taken together with the atoms connecting them form a 3-to-8-membered carbocyclic or heterocyclic ring containing 1 or 2 heteroatoms independently selected from O, N, and S, wherein the ring is optionally substituted with one or more substituents each independently selected from hydroxy, oxo, C₁-C₆ alkoxy, NR¹¹R¹², ═NR¹³, COOC₁-C₆ alkyl, and CONR¹¹R¹²;

R¹³ is C₁-C₆ alkyl;

each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl;

each R¹⁶ is the same or different and is selected from H, C₁-C₆ alkyl, C₁-C₆ alkoxy, NR¹¹R¹², oxo, and ═NR¹³;

provided that if the compound of formula I comprises ring A and ring B; X³ is NH; X² is CH; and R¹⁰ is H,

then (i) if X¹ is O and R¹ is C₁-C₆ alkyl substituted with hydroxy, oxo, C₁-C₆ alkoxy, NR¹¹R¹², ═NR¹³, COOC₁-C₆ alkyl, and CONR¹¹R¹², then Y is not N, CF, CCl or CH, and (ii) if X¹ is S, then Y is not CH;

and provided that if R² and R⁴ are each isopropyl; X³ is NH; X² is CH; R¹⁰ is H; and R¹ is C₁-C₆ alkyl optionally substituted with hydroxy, C₁-C₆ alkoxy, NR¹¹R¹², —NR¹³, COOC₁-C₆ alkyl, or CONR¹¹R¹²,

then Y is not CH or CCl.

In some embodiments of the compound of Formula I, X¹ is O.

In some embodiments of the compound of Formula I, X¹ is S.

In some embodiments of the compound of Formula I, X¹ is NH.

In some embodiments of the compound of Formula I, X² is CR⁹.

In some embodiments of the compound of Formula I, X² is CH.

In some embodiments of the compound of Formula I, X² is N.

In some embodiments of the compound of Formula I, X³ is NH.

In some embodiments of the compound of Formula I, X³ is O.

In some embodiments of the compound of Formula I, X² is C(C₁-C₆ alkyl).

In some embodiments of the compound of Formula I, X³ and R² taken together with the atoms connecting them form a four-to-seven-membered heterocyclic ring optionally substituted with one or more R¹⁶.

In certain of the foregoing embodiments of Formula I, X³ and R² taken together with the atoms connecting them form a four-to-seven-membered heterocyclic ring optionally substituted with one or more H.

In some embodiments of the compound of Formula I, X³ and R⁴ taken together with the atoms connecting them form a four-to-seven-membered heterocyclic ring optionally substituted with one or more R¹⁶.

In certain of the foregoing embodiments of Formula I, X³ and R⁴ taken together with the atoms connecting them form a four-to-seven-membered heterocyclic ring optionally substituted with one or more C₁-C₆ alkyl.

In some embodiments of the compound of Formula I, Y is CR⁸.

In some embodiments of the compound of Formula I, Y is N.

In some embodiments of the compound of Formula I, R² is hydrogen.

In some embodiments of the compound of Formula I, R² is C₁-C₆ alkyl optionally substituted with hydroxy. For example, R² can be isopropyl or methyl.

In some embodiments of the compound of Formula I, R³ is hydrogen.

In some embodiments of the compound of Formula I, R³ is C₁-C₆ alkyl optionally substituted with hydroxy. For example, R³ can be isopropyl; or R³ can be methyl.

In some embodiments of the compound of Formula I, R⁴ is hydrogen.

In some embodiments of the compound of Formula I, R⁴ is C₁-C₆ alkyl optionally substituted with hydroxy. For example, R⁴ can be isopropyl; or R⁴ can be methyl.

In some embodiments of the compound of Formula I, R⁵ is hydrogen.

In some embodiments of the compound of Formula I, R⁵ is C₁-C₆ alkyl optionally substituted with hydroxy. For example, R⁵ can be isopropyl; or R⁵ can be methyl.

In some embodiments of the compound of Formula I, R² and R³ taken together with the carbons connecting them form ring A.

In certain of the foregoing embodiments of Formula I, ring A is

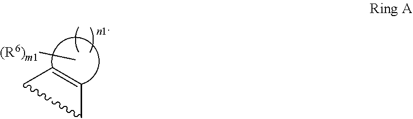

In some embodiments of the compound of Formula I, R² and R³ taken together with the carbons connecting them form ring B.

In certain of the foregoing embodiments of Formula I, ring B is

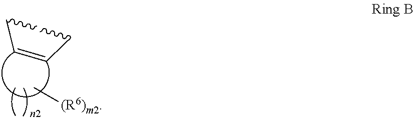

In certain embodiments of the compound of Formula I (when one pair R² and R³ taken together with the carbons connecting them form ring A; and another pair of R² and R³ taken together with the carbons connecting them form ring B), ring A is the same as ring B.

In some embodiments of the compound of Formula I, n1 is 3.

In some embodiments of the compound of Formula I, n1 is 4.

In some embodiments of the compound of Formula I, n2 is 3.

In some embodiments of the compound of Formula I, n2 is 4.

In some embodiments of the compound of Formula I, R⁶ is H.

In some embodiments of the compound of Formula I, R⁸ is H; R⁸ is CN; R⁸ is C₁; R⁸ is F; R⁸ is C₁-C₆ alkyl; or R⁸ is C₁-C₆ haloalkyl (e.g., CF₃).

In some embodiments of the compound of Formula I, R⁹ is H; R⁹ is CN; R⁹ is C₁; or R⁹ is F.

In some embodiments of the compound of Formula I, R¹ is H.

In some embodiments of the compound of Formula I, R¹ is C₁-C₆ alkyl optionally substituted with one or more substituents each independently selected from hydroxy, amino and oxo.

In certain embodiments of the compound of Formula I, R¹ is C₁-C₆ alkyl substituted with hydroxy. For example, R¹ can be 2-hydroxy-2-propyl.

In some embodiments of the compound of Formula I, R¹ is C₃-C₆ cycloalkyl optionally substituted with one or more substituents each independently selected from hydroxy, amino and oxo.

In certain embodiments of the compound of Formula I, R¹ is C₃-C₆ cycloalkyl substituted with hydroxy. For example, R¹ can be 1-hydroxy-1-cyclopropyl; R¹ can be 1-hydroxy-1-cyclobutyl; or R¹ can be 1-hydroxy-1-cyclopentyl.

In some embodiments of the compound of Formula I, R¹⁰ is H.

In some embodiments of the compound of Formula I, R¹⁰ is C₁-C₆ alkyl optionally substituted with one or more substituents each independently selected from hydroxy, amino and oxo.

In certain embodiments of the compound of Formula I, R¹⁰ is C₁-C₆ alkyl substituted with hydroxy. For example, R¹⁰ can be 2-hydroxy-2-propyl.

In some embodiments of the compound of Formula I, R¹⁰ is C₃-C₆ cycloalkyl optionally substituted with one or more substituents each independently selected from hydroxy, amino and oxo.

In certain embodiments of the compound of Formula I, R¹⁰ is C₃-C₆ cycloalkyl substituted with hydroxy. For example, R¹⁰ can be 1-hydroxy-1-cyclopropyl; R¹⁰ can be 1-hydroxy-1-cyclobutyl; or R¹⁰ can be 1-hydroxy-1-cyclopentyl.

In some embodiments of the compound of Formula I, R¹ and R¹⁰ taken together with the atoms connecting them form a five-membered carbocyclic ring.

In some embodiments of the compound of Formula I, R¹ and R¹⁰ taken together with the atoms connecting them form a six-membered carbocyclic ring.

In some embodiments of the compound of Formula I, R¹ and R¹⁰ taken together with the atoms connecting them form a five-membered heterocyclic ring containing 1 or 2 heteroatoms independently selected from O, N, and S.

In some embodiments of the compound of Formula I, R¹ and R¹⁰ taken together with the atoms connecting them form a six-membered heterocyclic ring containing 1 or 2 heteroatoms independently selected from O, N, and S.

In some embodiments of the compound of Formula I, the ring formed by R¹ and R¹⁰ is substituted with hydroxy.

In some embodiments of the compound of Formula I, the ring formed by R¹ and R¹⁰ is substituted with oxo.

In some embodiments of the compound of Formula I, the ring formed by R¹ and R¹⁰ is substituted with C₁-C₆ alkoxy.

In some embodiments of the compound of Formula I, each R¹¹ is hydrogen.

In some embodiments of the compound of Formula I, each R¹¹ is C₁-C₆ alkyl.

In some embodiments of the compound of Formula I, each R¹² is hydrogen.

In some embodiments of the compound of Formula I, each R¹² is C₁-C₆ alkyl.

In some embodiments of the compound of Formula I, each R¹⁶ is hydrogen.

In some embodiments of the compound of Formula I, if the compound of Formula I comprises ring A and ring B; X³ is NH; X² is CH; and R¹⁰ is H,

then (i) if X¹ is O and R¹ is C₁-C₆ alkyl substituted with hydroxy, C₁-C₆ alkoxy, NR¹¹R¹², —NR¹³, COOC₁-C₆ alkyl, and CONR¹¹R¹², then Y is not N, CF, CCl or CH, (ii) if X¹ is O and R¹ is C₁-C₆ alkyl substituted with oxo, then Y is not CH, and (iii) if X¹ is S, then Y is not CH;

and if R² and R⁴ are each isopropyl; X³ is NH; X² is CH; R¹⁰ is H; and R¹ is optionally substituted C₁-C₆ alkyl, then Y is not CH or CCl.

In some embodiments of the compound of Formula I, if the compound of formula I comprises ring A and ring B; X³ is NH; X² is CH; and R¹⁰ is H,

then (i) if X¹ is O and R¹ is C₁-C₆ alkyl substituted with hydroxy, oxo, C₁-C₆ alkoxy, NR¹¹R¹², ═NR¹³, COOC₁-C₆ alkyl, and CONR¹¹R¹², then Y is not N, CF, CCl or CH, and (ii) if X¹ is S, then Y is not CH;

and if R² and R⁴ are each isopropyl; X³ is NH; X² is CH; R¹⁰ is H; and R¹ is optionally substituted C₁-C₆ alkyl,

then Y is not CH or CCl.

In some embodiments of the compound of Formula I, the compound of Formula I is a compound of Formula Ia

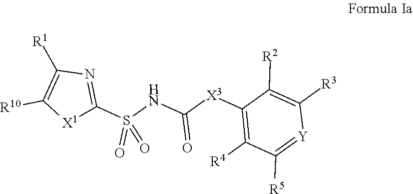

or a pharmaceutically acceptable salt thereof, wherein:

X¹ is O, S, or NH;

X³ is NH or O;

or when X³ is NH, X³ and R² taken together with the atoms connecting them form a four-to-seven-membered heterocyclic ring optionally substituted with one or more R¹⁶;

or when X³ is NH, X³ and R⁴ taken together with the atoms connecting them form a four-to-seven-membered heterocyclic ring optionally substituted with one or more R¹⁶;

Y is N or CR⁸;

R⁸ is selected from H, CN, Cl, F, CO₂C₁-C₆ alkyl and CONH₂;

R² is hydrogen, C₁-C₆ alkoxy, halo, C₁-C₆ haloalkyl, or C₁-C₆ alkyl optionally substituted with hydroxy;

R³ is hydrogen, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

provided that at least one of R², R³, R⁴ and R⁵ is not hydrogen, and that R² and R⁴ are not both hydroxymethyl;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

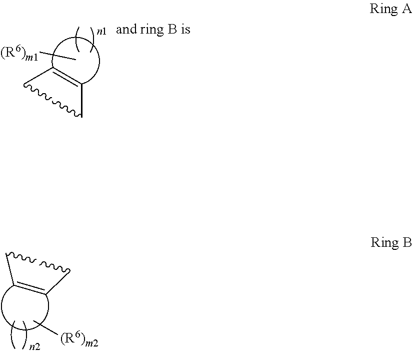

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from H, C₁-C₆ alkyl and C₃-C₆ cycloalkyl, wherein R¹ is optionally substituted with hydroxy, amino or oxo;

R¹⁰ is selected from H, C₁-C₆ alkyl and C₃-C₆ cycloalkyl, wherein R¹⁰ is optionally substituted with hydroxy, amino or oxo;

wherein R¹⁰ is optionally substituted with one or more substituents each, independently selected from hydroxy, oxo, C₁-C₆ alkoxy, NR¹¹R¹², ═NR¹³, COOC₁-C₆ alkyl, and CONR¹¹R¹²;

or R¹ and R¹⁰ taken together with the atoms connecting them form a 3-to-8-membered carbocyclic or heterocyclic ring containing 1 or 2 heteroatoms independently selected from O, N, and S, wherein the ring is optionally substituted with one or more substituents each independently selected from hydroxy, oxo, C₁-C₆ alkoxy, NR¹¹R¹² ═NR¹³, COOC₁-C₆ alkyl, and CONR¹¹R¹²;

═R¹³ is C₁-C₆ alkyl;

each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl;

each R¹⁶ is the same or different and is selected from H, C₁-C₆ alkyl, C₁-C₆ alkoxy, NR¹¹R¹², oxo, and ═NR¹³.

In some embodiments of the compound of Formula I, the compound of Formula I is a compound of Formula Ia

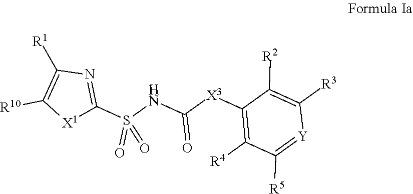

or a pharmaceutically acceptable salt thereof,

wherein

X¹ is O, S, or NH;

X³ is NH or O;

or when X³ is NH, X³ and R² taken together with the atoms connecting them form a four-to-seven-membered heterocyclic ring optionally substituted with one or more R¹⁶;

or when X³ is NH, X³ and R⁴ taken together with the atoms connecting them form a four-to-seven-membered heterocyclic ring optionally substituted with one or more R¹⁶;

Y is N or CR⁸;

R⁸ is selected from H, CN, Cl, F, CO₂C₁-C₆ alkyl and CONH₂;

R² is hydrogen or C₁-C₆ alkyl optionally substituted with hydroxy;

R³ is hydrogen or C₁-C₆ alkyl optionally substituted with hydroxy;

R⁴ is hydrogen or C₁-C₆ alkyl optionally substituted with hydroxy and is the same as R²;

R⁵ is hydrogen or C₁-C₆ alkyl optionally substituted with hydroxy and is the same as R³;

provided that at least one of R², R³, R⁴ and R⁵ is not hydrogen, and that R² and R⁴ are not both hydroxymethyl;

or R² and R³ taken together with the carbons connecting them form a five-membered ring A and R⁴ and R⁵ taken together with the carbons connecting them form a five-membered ring B,

wherein ring A is

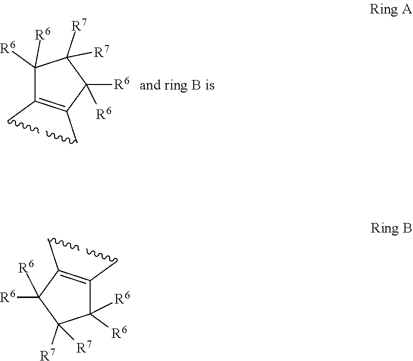

wherein each R⁶ in each ring is the same and is H or C₁-C₆ alkyl, and each R⁷ in each ring is the same and is H or C₁-C₆ alkyl;

R¹ is selected from H, C₁-C₆ alkyl and C₃-C₆ cycloalkyl, wherein R¹ is optionally substituted with one or more substituents each independently selected from hydroxy, amino and oxo;

R¹⁰ is selected from H, C₁-C₆ alkyl and C₃-C₆ cycloalkyl, wherein R¹⁰ is optionally substituted with one or more substituents each independently selected from hydroxy, amino and oxo;

or R¹ and R¹⁰ taken together with the atoms connecting them form a five-membered, a six-membered, or a seven-membered carbocyclic or heterocyclic ring.

In some embodiments of the compound of Formula I, the compound is a compound of Formula Ia-i:

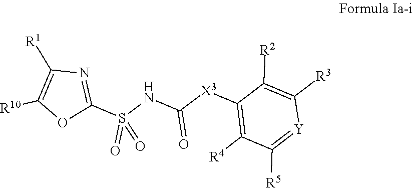

or a pharmaceutically acceptable salt thereof,

wherein:

X³ is NH or O;

Y is N or CR⁸;

R⁸ is selected from H, CN, Cl, F, CO₂C₁-C₆ alkyl and CONH₂;

R² is C₁-C₆ alkoxy, halo, C₁-C₆ haloalkyl, or C₁-C₆ alkyl optionally substituted with hydroxy;

R³ is hydrogen;

R⁴ is C₁-C₆ alkoxy, halo, C₁-C₆ haloalkyl, or C₁-C₆ alkyl optionally substituted with hydroxy;

R⁵ is hydrogen;

R¹ is selected from H, C₁-C₆ alkyl and C₃-C₆ cycloalkyl, wherein R¹ is optionally substituted with hydroxy, amino or oxo;

R¹⁰ is selected from H, C₁-C₆ alkyl and C₃-C₆ cycloalkyl, wherein R¹⁰ is optionally substituted with hydroxy, amino or oxo;

or R¹ and R¹⁰ taken together with the atoms connecting them form a five-membered, a six-membered, or a seven-membered carbocyclic or heterocyclic ring.

In some embodiments of the compound of Formula I, the compound is a compound of Formula Ia-i:

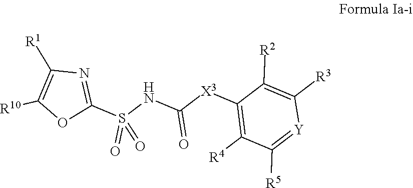

or a pharmaceutically acceptable salt thereof,

wherein:

X³ is NH or O;

Y is N or CR⁸;

R⁸ is selected from H, CN, Cl, F, CO₂C₁-C₆ alkyl and CONH₂;

R³ is C₁-C₆ alkoxy, halo, C₁-C₆ haloalkyl, or C₁-C₆ alkyl optionally substituted with hydroxy;

R² is hydrogen;

R⁵ is C₁-C₆ alkoxy, halo, C₁-C₆ haloalkyl, or C₁-C₆ alkyl optionally substituted with hydroxy;

R⁴ is hydrogen;

R¹ is selected from H, C₁-C₆ alkyl and C₃-C₆ cycloalkyl, wherein R¹ is optionally substituted with hydroxy, amino or oxo;

R¹⁰ is selected from H, C₁-C₆ alkyl and C₃-C₆ cycloalkyl, wherein R¹⁰ is optionally substituted with hydroxy, amino or oxo;

or R¹ and R¹⁰ taken together with the atoms connecting them form a five-membered, a six-membered, or a seven-membered carbocyclic or heterocyclic ring.

In some embodiments of the compound of Formula I, the compound is a compound of Formula Ia-i:

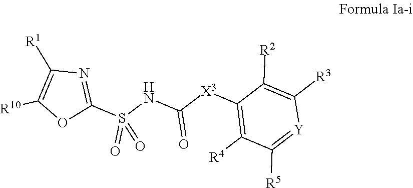

or a pharmaceutically acceptable salt thereof,

wherein:

X³ is NH or O;

Y is N or CR⁸;

R⁸ is selected from H, CN, Cl, F, CO₂C₁-C₆ alkyl and CONH₂;

R² and R³ taken together with the carbons connecting them form a four-membered to seven-membered ring A and R⁴ and R⁵ taken together with the carbons connecting them form a four-membered to seven-membered ring B,

wherein ring A is

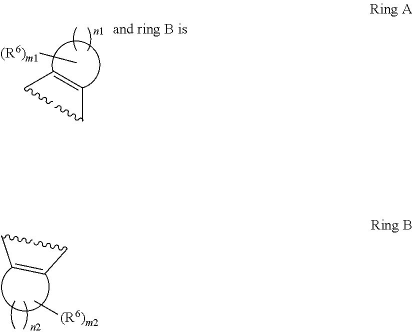

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from H, C₁-C₆ alkyl and C₃-C₆ cycloalkyl, wherein R¹ is optionally substituted with hydroxy, amino or oxo;

R¹⁰ is selected from H, C₁-C₆ alkyl and C₃-C₆ cycloalkyl, wherein R¹⁰ is optionally substituted with hydroxy, amino or oxo;

or R¹ and R¹⁰ taken together with the atoms connecting them form a five-membered, a six-membered, or a seven-membered carbocyclic or heterocyclic ring.

In some embodiments of the compound of Formula I, the compound is a compound of Formula Ia-i(A):

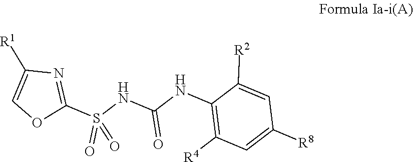

or a pharmaceutically acceptable salt thereof, wherein R⁸ is H, CN, F, CO₂C₁-C₆ alkyl, or CONH₂.

In some embodiments of the compound of Formula I, the compound is a compound of Formula Ia-i(B):

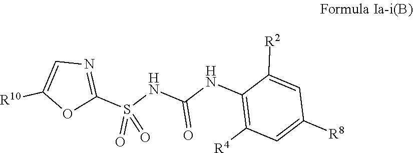

or a pharmaceutically acceptable salt thereof, wherein R⁸ is H, CN, F, CO₂C₁-C₆ alkyl, or CONH₂.

In some embodiments of the compound of Formula I, the compound of Formula Ia is a compound of Formula Ia-i(C):

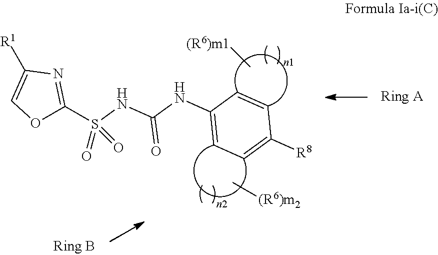

or a pharmaceutically acceptable salt thereof, wherein R⁸ is H, CN, C₁ or F.

In some embodiments of the compound of Formula I, the compound of Formula Ia is a compound of Formula Ia-i(D):

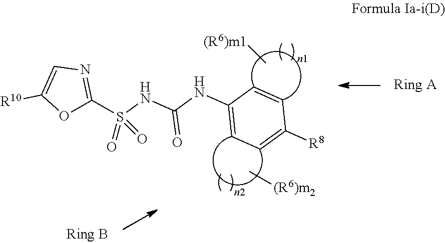

or a pharmaceutically acceptable salt thereof, wherein R⁸ is H, CN, C₁ or F.

In some embodiments of the compound of Formula I, the compound of Formula Ia is a compound of Formula Ia-i(E):

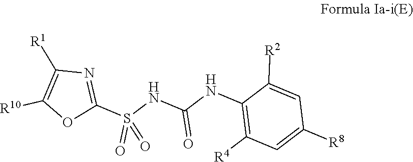

or a pharmaceutically acceptable salt thereof, wherein R⁸ is H, CN, F, CO₂C₁-C₆ alkyl, or CONH₂.

In some embodiments of the compound of Formula I, the compound of Formula Ia is a compound of Formula Ia-i(F):

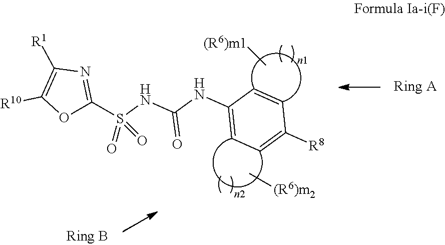

or a pharmaceutically acceptable salt thereof, wherein R⁸ is H, CN, C₁ or F.

In some embodiments of the compound of Formula I, the compound of Formula Ia is a compound of Formula Ia-i(G):

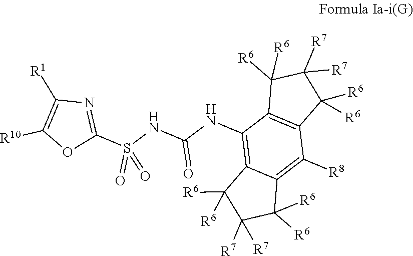

or a pharmaceutically acceptable salt thereof, wherein R⁸ is H, CN, C₁ or F.

In another aspect, an NLRP3 antagonist is a compound of Formula II,

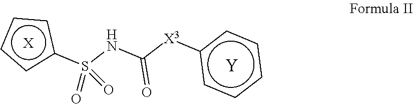

or a pharmaceutically acceptable salt thereof, wherein Formula II is selected from

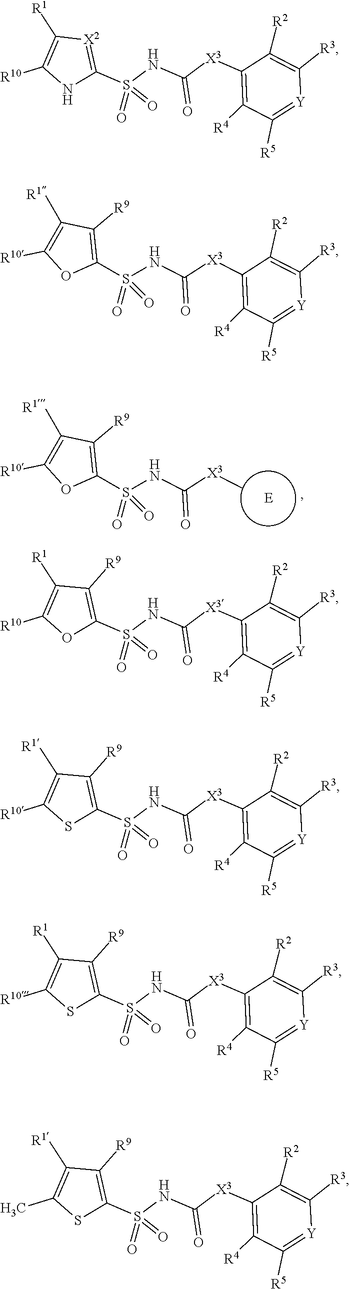

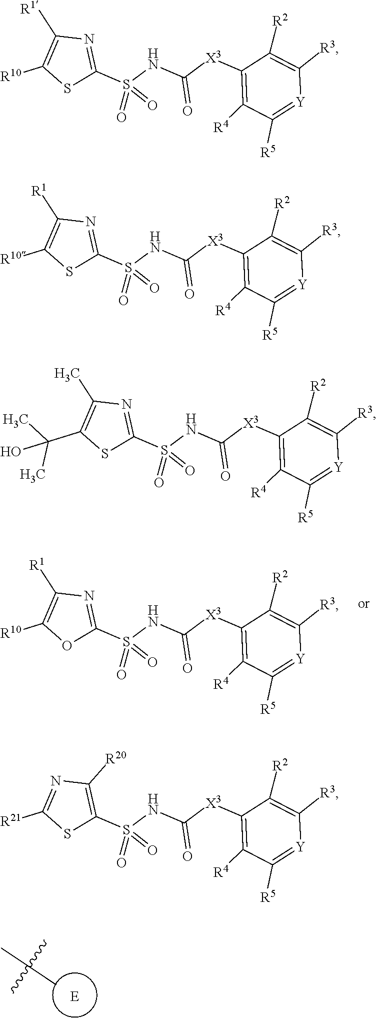

is selected from

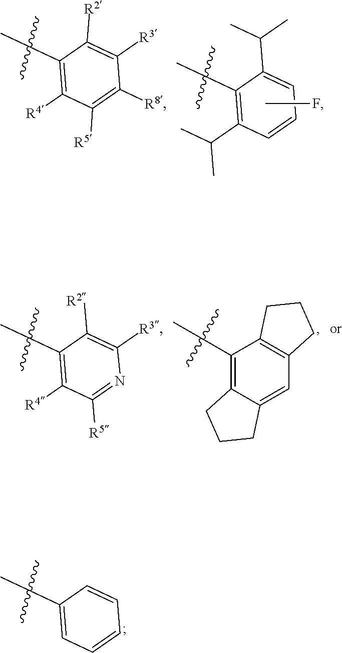

X² is N or CR⁹;

X³ is NH or O;

X^{3′} is O;

or when X³ is NH, X³ and R² taken together with the atoms connecting them form a four-to-seven-membered heterocyclic ring optionally substituted with one or more R¹⁶; or when X³ is NH, X³ and R⁴ taken together with the atoms connecting them form a four-to-seven-membered heterocyclic ring optionally substituted with one or more R¹⁶;

Y is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, C₁-C₆ haloalkoxy, and C₁-C₆ haloalkyl;

R^{8′} is selected from CN, CO₂C₁-C₆ alkyl, CO₂C₃-C₈ cycloalkyl, CONR¹¹R¹², and C₁-C₆ haloalkyl;

R⁹ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R^{2′} is hydrogen, halo, or C₁-C₆ alkyl optionally substituted with hydroxy;

R^{2″} is hydrogen or C₁-C₆ alkyl;

R³ is hydrogen, CN, C₁-C₆ alkoxy, halo, C₁-C₆ haloalkyl, or C₁-C₆ alkyl optionally substituted with hydroxy;

R^{3′} is hydrogen, halo, or C₁-C₆ alkyl optionally substituted with hydroxy;

R^{3″} is hydrogen, CN, or C₁-C₆ alkyl;

R⁴ is hydrogen, C₁-C₆ alkoxy, halo, C₁-C₆ haloalkyl, or C₁-C₆ alkyl optionally substituted with hydroxy;

R^{4′} is hydrogen, halo, or C₁-C₆ alkyl optionally substituted with hydroxy;

R^{4″} is hydrogen or C₁-C₆ alkyl;

R⁵ is hydrogen, C₁-C₆ alkoxy, halo, C₁-C₆ haloalkyl, or C₁-C₆ alkyl optionally substituted with hydroxy;

R^{5′} is hydrogen, halo, or C₁-C₆ alkyl optionally substituted with hydroxy;

R^{5″} is hydrogen, CN, or C₁-C₆ alkyl;

or R² and R³ taken together with the carbons connecting them form a four-membered to seven-membered ring A,

or R^{2′} and R³ taken together with the carbons connecting them form a four-membered to seven-membered ring A,

or R⁴ and R⁵ taken together with the carbons connecting them form a four-membered to seven-membered ring B,

or R^{4′} and R^{5′} taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

or R^{2′} and R^{3′} taken together with the carbons connecting them form a four-membered to seven-membered ring A and R^{4′} and R^{5′} taken together with the carbons connecting them form a four-membered to seven-membered ring B,

wherein ring A is

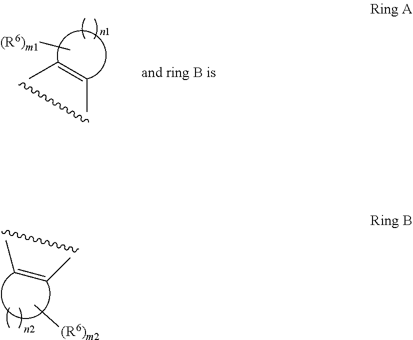

wherein

ring A is a saturated carbocyclic ring;

n1 is from 2 to 5;

m1 is from 1 to 10;

wherein ring B is a saturated carbocyclic ring;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;
or two R⁶ taken together with the atom or atoms connecting them form a 3-to-8-membered carbocyclic or saturated heterocyclic ring containing 1 or 2 heteroatoms independently selected from O, N, and S;

R¹ is selected from H, unsubstituted C₁-C₆ alkyl, C(R¹⁹)₂OH, C(O)C₂-C₆ alkyl, and C₃-C₆ cycloalkyl;

wherein each C(O)C₂-C₆ alkyl and C₃-C₆ cycloalkyl above is optionally substituted with one or more substituents each independently selected from hydroxy, C₁-C₆ alkoxy, ═NR¹³, COOC₁-C₆ alkyl, and CONR¹¹R¹²,

R^{1′} is selected from unsubstituted C₁-C₆ alkyl, C(R¹⁹)₂OH, C(O)C₂-C₆ alkyl, and C₃-C₆ cycloalkyl;

wherein each C(O)C₂-C₆ alkyl and C₃-C₆ cycloalkyl above is optionally substituted with one or more substituents each independently selected from hydroxy, C₁-C₆ alkoxy, NR¹¹R¹², ═NR¹³, COOC₁-C₆ alkyl, and CONR¹¹R¹²,

R¹ is selected from unsubstituted C₁-C₆ alkyl, C(O)C₂-C₆ alkyl, and C₃-C₆ cycloalkyl;
wherein each C(O)C₂-C₆ alkyl and C₃-C₆ cycloalkyl above is optionally substituted with one or more substituents each independently selected from hydroxy, oxo, C₁-C₆ alkoxy, NR¹¹R¹², ═NR¹³, COOC₁-C₆ alkyl, and CONR¹¹R¹²;

R^{1″″} is selected from C(R¹⁹)₂OH;

R¹⁰ is selected from H, Cl, C₁-C₆ alkyl, C₃-C₆ cycloalkyl and C₃-C₆ heterocycloalkyl;
wherein R¹⁰ is optionally substituted with one or more substituents each independently selected from hydroxy, C₁-C₆ alkoxy, NR¹¹R¹²; ═NR¹³, COOC₁-C₆ alkyl, and CONR¹¹R¹²;
R^{10′} is selected from H, Cl, C₃-C₆ cycloalkyl and C₃-C₆ heterocycloalkyl;

wherein R^{ill} is optionally substituted with one or more substituents each, independently selected from hydroxy, oxo, C₁-C₆ alkoxy, R¹¹R¹², ═NR¹³, COOC₁-C₆ alkyl, and CONR¹¹R¹²;
R^{10″} is selected from C₁, C₁-C₆ alkyl, C₃-C₆ cycloalkyl, and C₃-C₆ heterocycloalkyl;

wherein R^{10″} is optionally substituted with one or more substituents each independently, selected from hydroxy, C₁-C₆ alkoxy, R¹¹R¹², ═NR¹³, COOC₁-C₆ alkyl, and CONR¹¹R¹²;

R^{10″′} is selected from C₁, C₁-C₆ alkyl substituted with hydroxy, C₃-C₆ cycloalkyl, and C₃-C₆ heterocycloalkyl;

wherein the C₃-C₆ cycloalkyl, and C₃-C₆ heterocycloalkyl above are each optionally substituted with one or more substituents each independently selected from hydroxy, oxo, C₁-C₆ alkoxy, NR¹¹R¹²; ═NR¹³, COOC₁-C₆ alkyl, and CONR¹¹R¹²;

or R¹ and R¹⁰ taken together with the atoms connecting them form a 3-to-8-membered carbocyclic or heterocyclic ring containing 1 or 2 heteroatoms independently selected from O, N, and S, wherein the ring is optionally substituted with one or more substituents each independently selected from hydroxy, oxo, C₁-C₆ alkoxy, NR¹¹R¹²; ═NR¹³, COOC₁-C₆ alkyl, and CONR¹¹R¹²;

R¹³ is C₁-C₆ alkyl;

each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl;

each R¹⁶ is the same or different and is selected from H, C₁-C₆ alkyl, C₁-C₆ alkoxy, NR¹¹R¹², oxo, and ═NR¹³;

each R¹⁹ is the same and is C₁-C₆ alkyl;

R²⁰ is selected from H, halo, or C₁-C₆ alkyl optionally substituted with hydroxy;

R²¹ is selected from H, halo, or C₁-C₆ alkyl substituted with hydroxy;

provided that if (1) Formula II is

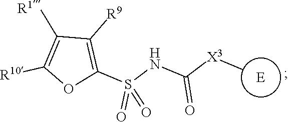

and if (2) R^{10′} is H or C₃-C₆ heterocycloalkyl; then

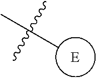

is not

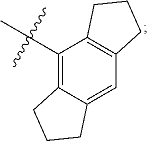 [SMILES: *c1c2c(cc3c1CCC3)CCC2]

and provided that if: (1) Formula II is

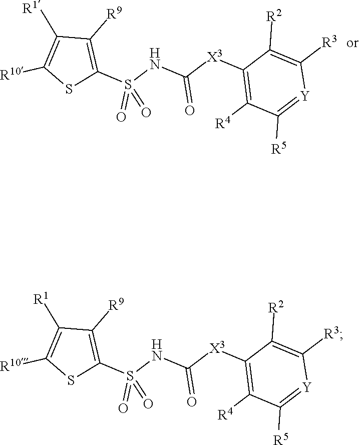

and (2) either of R¹ or R^{1′}, when present, is C(R¹⁹)₂OH; and (3) either of R^{10′} or R^{10′″}, when present, is not C₁; then

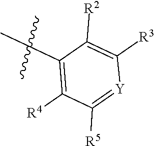

is not

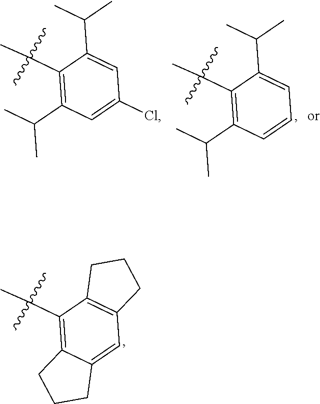

and provided that if: (1) Formula II is

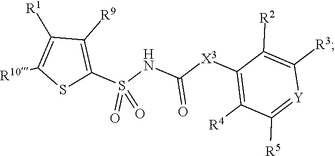

and (2) R^{10′″} is C₁-C₆ alkyl substituted with hydroxy; then

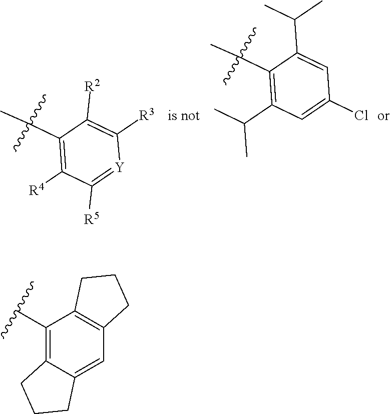

In another aspect, an NLRP3 antagonist is a compound of Formula II,

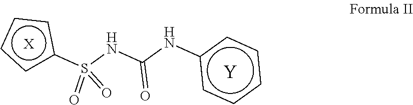

or a pharmaceutically acceptable salt thereof, wherein Formula II is selected from

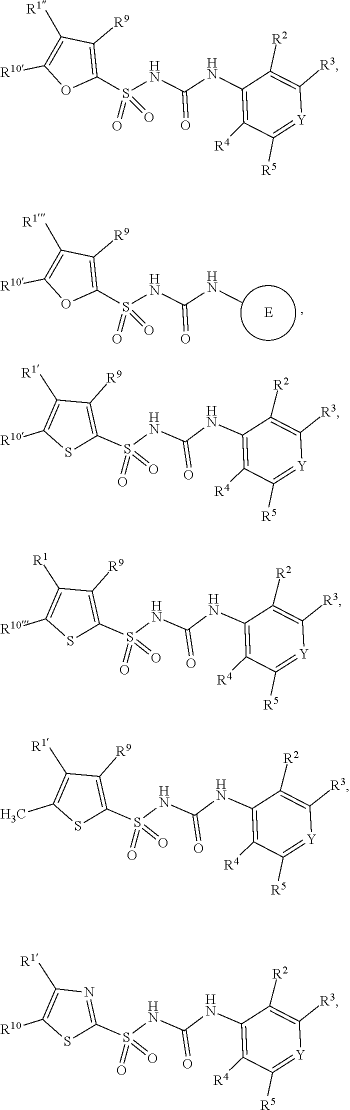

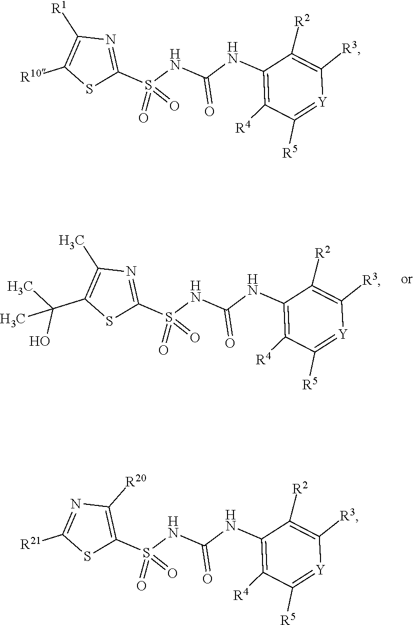

is selected from

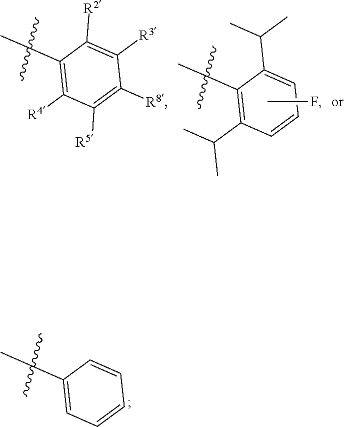

Y is N or CR⁸;

R⁸ is selected from H, CN, Cl, and F;

R^{8′} is selected from CN and CONR¹¹R¹²;

R⁹ is selected from H;

R² is hydrogen or C₁-C₆ alkyl;

R^{2′} is C₁-C₆ alkyl;

R³ is hydrogen, CN, C₁-C₆ alkoxy, or halo;

R^{3′} is hydrogen or halo;

R⁴ is hydrogen or C₁-C₆ alkyl;

R^{4′} is C₁-C₆ alkyl;

R⁵ is hydrogen;

R^{5′} is hydrogen;

or R² and R³ taken together with the carbons connecting them form a four-membered to seven-membered ring A,

or R^{2′} and taken together with the carbons connecting them form a four-membered to seven-membered ring A,

or R⁴ and R⁵ taken together with the carbons connecting them form a four-membered to seven-membered ring B,

or R^{4′} and R^{5′} taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

or R^{2′} and taken together with the carbons connecting them form a four-membered to seven-membered ring A and R^{4′} and R^{5′} taken together with the carbons connecting them form a four-membered to seven-membered ring B,

wherein ring A is

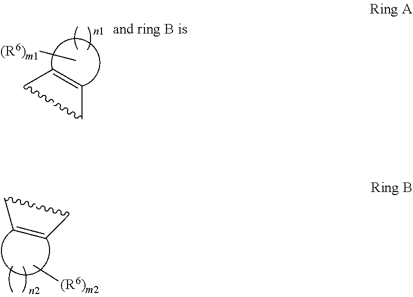

wherein

ring A is a saturated carbocyclic ring;

n1 is 3;

m1 is 0;

wherein ring B is a saturated carbocyclic ring;

n2 is 3;

m2 is 0;

R¹ is selected from H;

R^{1′} is selected from C(R¹⁹)₂OH and C₃-C₆ cycloalkyl;

wherein the C₃-C₆ cycloalkyl above is optionally substituted with one or more hydroxy;

R¹ is selected from C₃-C₆ cycloalkyl;

wherein the C₃-C₆ cycloalkyl above is optionally substituted with one or more hydroxy;

R^{1′″} is selected from C(R¹⁹)₂OH;

R¹⁰ is selected from H, Cl, C₁-C₆ alkyl, and C₃-C₆ cycloalkyl;

wherein R^{10′} is optionally substituted with one or more substituents each independently selected from hydroxy;

R^{10′} is selected from H or C₁;

R^{10″} is selected from C₁-C₆ alkyl, and C₃-C₆ cycloalkyl;

wherein R^{10″} is optionally substituted with one or more hydroxy;

R^{10′″} is selected from C₁-C₆ alkyl and C₃-C₆ cycloalkyl;

wherein R^{10′″} is optionally substituted with one or more hydroxy;

each of R¹¹ and R¹² at each occurrence is independently selected from hydrogen;

each R¹⁹ is the same and is selected from C₁-C₆ alkyl;

R²⁰ is selected from H or C₁-C₆ alkyl optionally substituted with hydroxy;

R²¹ is selected from H or C₁-C₆ alkyl substituted with hydroxy;

provided that if: (1) Formula II is

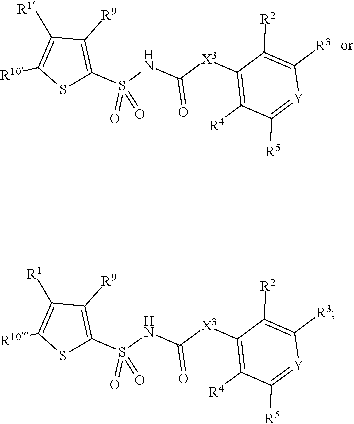

and (2) either of R¹ or R^{1′}, when present, is C(R¹⁹)₂OH; and (3) either of R^{10′} or R^{10′″}, when present, is not C₁; then

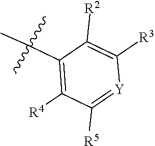

is not

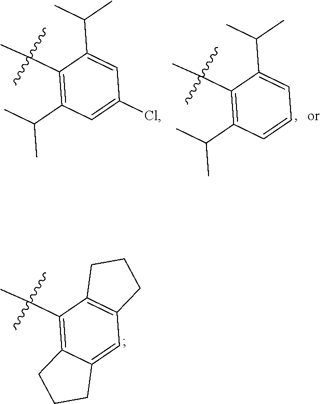

and provided that if: (1) Formula II is

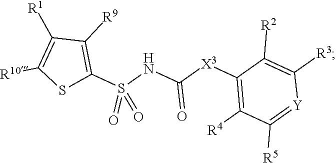

and if (2) R^{10′″} is C₁-C₆ alkyl substituted with hydroxy; then

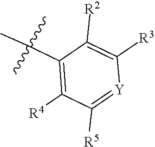

is not

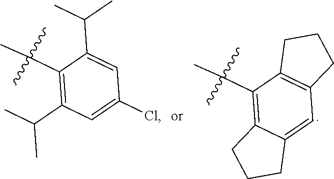 [SMILES: *c1c(C(C)C)cc(Cl)cc1C(C)C ; *c1c2c(cc3c1CCC3)CCC2]

In some embodiments of the compound of Formula II, the compound of Formula II is

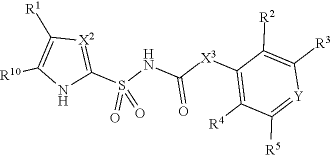

or a pharmaceutically acceptable salt thereof, and wherein:

X² is N or CR⁹;

X³ is NH or O;

or when X³ is NH, X³ and R² taken together with the atoms connecting them form a four-to-seven-membered heterocyclic ring optionally substituted with one or more R¹⁶;

or when X³ is NH, X³ and R⁴ taken together with the atoms connecting them form a four-to-seven-membered heterocyclic ring optionally substituted with one or more R¹⁶;

Y is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, C₁-C₆ haloalkoxy, and C₁-C₆ haloalkyl;

R⁹ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R³ is hydrogen, CN, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

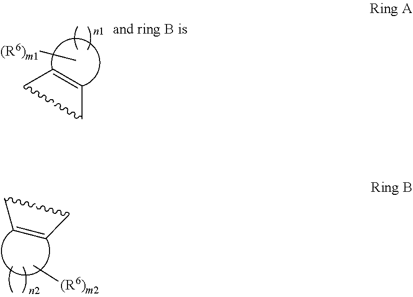

wherein

ring A is a saturated carbocyclic ring;

n1 is from 2 to 5;

m1 is from 1 to 10;

wherein ring B is a saturated carbocyclic ring;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from H, unsubstituted C₁-C₆ alkyl, C(R¹⁹)₂OH, C(O)C₂-C₆ alkyl, and C₃-C₆ cycloalkyl;

wherein each C(O)C₂-C₆ alkyl and C₃-C₆ cycloalkyl above is optionally substituted with one or more substituents each independently selected from hydroxy, C₁-C₆ alkoxy, NR¹¹R¹², ═NR¹³, COOC₁-C₆ alkyl, and CONR¹¹R¹²;

R¹⁰ is selected from H, Cl, C₁-C₆ alkyl, C₃-C₆ cycloalkyl and C₃-C₆ heterocycloalkyl;

wherein R¹⁰ is optionally substituted with one or more substituents each independently selected from hydroxy, C₁-C₆ alkoxy, NR¹¹R¹²; ═NR¹³; COOC₁-C₆ alkyl, and CONR¹¹R¹²;

or R¹ and R¹⁰ taken together with the atoms connecting them form a 3-to-8-membered carbocyclic or heterocyclic ring containing 1 or 2 heteroatoms independently selected from O, N, and S, wherein the ring is optionally substituted with one or more substituents each independently selected from hydroxy, oxo, C₁-C₆ alkoxy, NR¹¹R¹²; ═NR¹³, COOC₁-C₆ alkyl, and CONR¹¹R¹²;

R¹³ is C₁-C₆ alkyl;

each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl;

each R¹⁶ is the same or different and is selected from H, C₁-C₆ alkyl, C₁-C₆ alkoxy, NR¹¹R¹², oxo, and ═NR¹³;

each R¹⁹ is the same and is selected from C₁-C₆ alkyl.

In some embodiments of the compound of Formula II, X² is CR⁹.

In some embodiments of the compound of Formula II, the compound of Formula II is

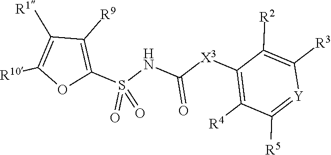

or a pharmaceutically acceptable salt thereof, and wherein:

X³ is NH or O;

or when X³ is NH, X³ and R² taken together with the atoms connecting them form a four-to-seven-membered heterocyclic ring optionally substituted with one or more R¹⁶; or when X³ is NH, X³ and R⁴ taken together with the atoms connecting them form a four-to-seven-membered heterocyclic ring optionally substituted with one or more R¹⁶;

Y is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, C₁-C₆ haloalkoxy, and C₁-C₆ haloalkyl;

R⁹ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R³ is hydrogen, CN, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

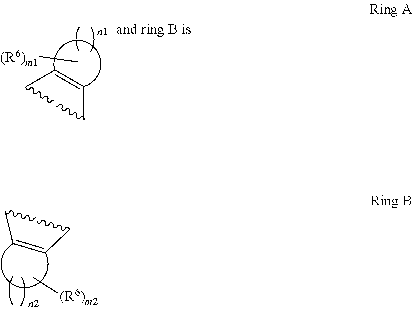

wherein

ring A is a saturated carbocyclic ring;

n1 is from 2 to 5;

m1 is from 1 to 10;

wherein ring B is a saturated carbocyclic ring;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from unsubstituted C₁-C₆ alkyl, C(O)C₂-C₆ alkyl, and C₃-C₆ cycloalkyl;
wherein each C(O)C₂-C₆ alkyl and C₃-C₆ cycloalkyl above is optionally substituted with one or more substituents each independently selected from hydroxy, oxo, C₁-C₆ alkoxy, NR¹¹R¹², ═NR¹³, COOC₁-C₆ alkyl, and CONR¹¹R¹²;
R^{10′} is selected from H, Cl, C₃-C₆ cycloalkyl and C₃-C₆ heterocycloalkyl;

wherein R^{ill} is optionally substituted with one or more substituents each independently selected from hydroxy, oxo, C₁-C₆ alkoxy, NR¹¹R¹², ═NR¹³ cooc₁-C₆ alkyl, and CONR¹¹R¹²;
R¹³ is C₁-C₆ alkyl;

each of R¹¹ and R¹² at each occurrence is independently selected from hydrogen, C₁-C₆ alkyl, CO₂R¹⁵ and CONR¹⁷R¹⁸; R¹⁵ is C₁-C₆ alkyl;

each of R¹⁷ and R¹⁸ at each occurrence is independently selected from hydrogen and C₁-C₆ alkyl;
each R¹⁶ is the same or different and is selected from H, C₁-C₆ alkyl, C₁-C₆ alkoxy, NR¹¹R¹², oxo, and ═NR¹³.

In some embodiments of the compound of Formula II, R¹ is 1-hydroxycyclopropyl and R^{10′} is H.

In some embodiments of the compound of Formula II, the compound of Formula II is

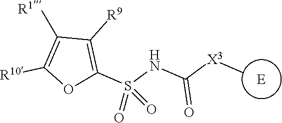

or a pharmaceutically acceptable salt thereof, and wherein:

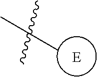

is selected from

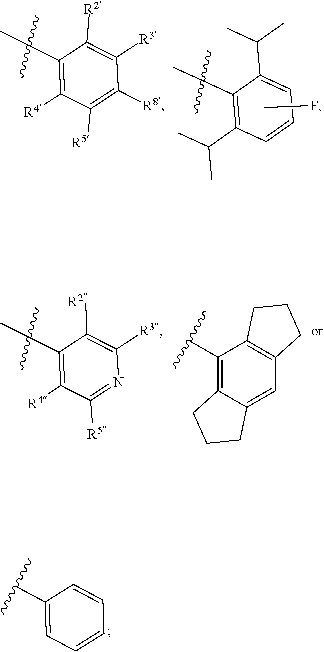

X³ is NH or O;

R^{8′} is selected from CN, CO₂C₁-C₆ alkyl, CO₂C₃-C₈ cycloalkyl, CONR¹¹R¹², C₁-C₆ haloalkoxy, and C₁-C₆ haloalkyl;

R⁹ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R^{2′} is hydrogen, halo, or C₁-C₆ alkyl optionally substituted with hydroxy;

R²″ is hydrogen or C₁-C₆ alkyl;

R^{3′} is hydrogen, halo, or C₁-C₆ alkyl optionally substituted with hydroxy;

R³″ is hydrogen, CN, or C₁-C₆ alkyl;

R^{4′} is hydrogen, halo, or C₁-C₆ alkyl optionally substituted with hydroxy;

R⁴″ is hydrogen or C₁-C₆ alkyl;

R^{5′} is hydrogen, halo, or C₁-C₆ alkyl optionally substituted with hydroxy;

R^{5″} is hydrogen, CN, or C₁-C₆ alkyl;

or R^{2′} and R³ taken together with the carbons connecting them form a four-membered to seven-membered ring A,

or R^{4′} and R^{5′} taken together with the carbons connecting them form a four-membered to seven-membered ring B,

or R^{2′} and R³ taken together with the carbons connecting them form a four-membered to seven-membered ring A and R^{4′} and R^{5′} taken together with the carbons connecting them form a four-membered to seven-membered ring B,

wherein ring A is

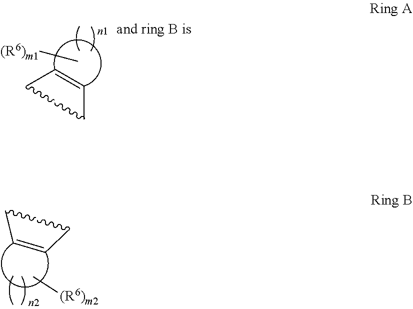

wherein

ring A is a saturated carbocyclic ring;

n1 is from 2 to 5;

m1 is from 1 to 10;

wherein ring B is a saturated carbocyclic ring;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R^{1′″} is selected from C(R¹⁹)₂OH;

R^{10′} is selected from H, Cl, C₃-C₆ cycloalkyl and C₃-C₆ heterocycloalkyl;

wherein R^{10′} is optionally substituted with one or more substituents each independently selected from hydroxy, oxo, C₁-C₆ alkoxy, NR¹¹R¹², ═NR¹³, COOC₁-C₆ alkyl, and CONR¹¹R¹²;

R¹³ is C₁-C₆ alkyl;

each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl;

each R¹⁶ is the same or different and is selected from H, C₁-C₆ alkyl, C₁-C₆ alkoxy, NR¹¹R¹², oxo, and ═NR¹³;

each R¹⁹ is the same and is selected from C₁-C₆ alkyl;

provided that if R^{10′} is H or C₃-C₆ heterocycloalkyl; then

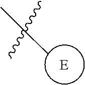

is not

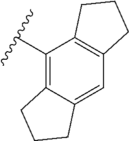 [SMILES: *c1c2c(cc3c1CCC3)CCC2]

In some embodiments of the compound of Formula II, R^{10′} is H.

In some embodiments of the compound of Formula II,

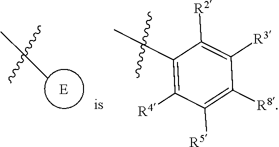

In some embodiments of the compound of Formula II, R^{2′} and R^{3′} taken together with the carbons connecting them form a four-membered to seven-membered ring A, and R^{4′} and R^{5′} taken together with the carbons connecting them form a four-membered to seven-membered ring B.

In some embodiments of the compound of Formula II, R^{8′} is CN.

In some embodiments of the compound of Formula II,

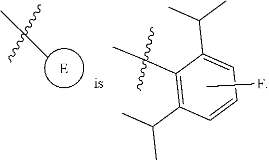

In some embodiments of the compound of Formula II,

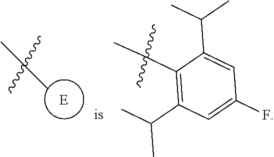

In some embodiments of the compound of Formula II,

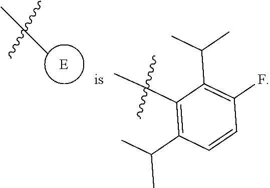

In some embodiments of the compound of Formula II, the compound of Formula II is

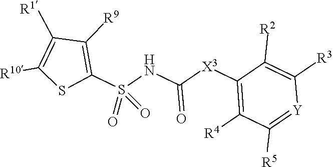

or a pharmaceutically acceptable salt thereof, and wherein:

X³ is NH or O;

or when X³ is NH, X³ and R² taken together with the atoms connecting them form a four-to-seven-membered heterocyclic ring optionally substituted with one or more R¹⁶;

or when X³ is NH, X³ and R⁴ taken together with the atoms connecting them form a four-to-seven-membered heterocyclic ring optionally substituted with one or more 10⁶;

Y is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, C₁-C₆ haloalkoxy, and C₁-C₆ haloalkyl;

R⁹ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R³ is hydrogen, CN, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

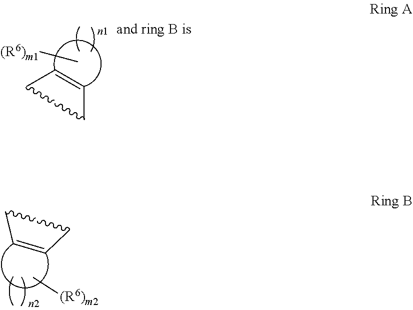

wherein

ring A is a saturated carbocyclic ring;

n1 is from 2 to 5;

m1 is from 1 to 10;

wherein ring B is a saturated carbocyclic ring;

n2 is from 2 to 5;

m2 is from 1 to 10;

wherein each R⁶ in each ring is the same or different and is selected from H, C₁-C₆ alkyl, C₁-C₆ alkoxy NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R^{1′} is selected from unsubstituted C₁-C₆ alkyl, C(R¹⁹)₂OH, C(O)C₂-C₆ alkyl, and C₃-C₆ cycloalkyl;

wherein each C(O)C₂-C₆ alkyl and C₃-C₆ cycloalkyl above is optionally substituted with one or more substituents each independently selected from hydroxy, C₁-C₆ alkoxy, NR¹¹R¹², ═NR¹³, COOC₁-C₆ alkyl, and CONR¹¹R¹²;

R^{10′} is selected from H, Cl, C₃-C₆ cycloalkyl and C₃-C₆ heterocycloalkyl;

wherein R^{10′} is optionally substituted with one or more substituents each independently selected from hydroxy, oxo, C₁-C₆ alkoxy, NR¹¹R¹², ═NR¹³, COOC₁-C₆ alkyl, and CONR¹¹R¹²;

R¹³ is C₁-C₆ alkyl;

each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl;

each R¹⁶ is the same or different and is selected from H, C₁-C₆ alkyl, C₁-C₆ alkoxy, N¹¹R¹², oxo, and ═NR¹³;

each R¹⁹ is the same and is selected from C₁-C₆ alkyl;

provided that if (1) R^{1′} is C(R¹⁹)₂OH; and (2) R^{10′} is not C₁; then

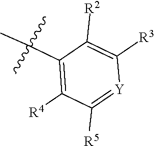

is not

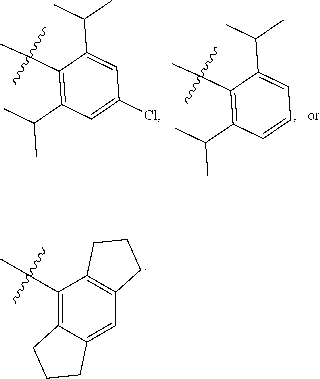

In some embodiments of the compound of Formula II, R^{1′} is C(R¹⁹)₂OH.

In some embodiments of the compound of Formula II, R^{10′} is H.

In some embodiments of the compound of Formula II, the compound of Formula II is

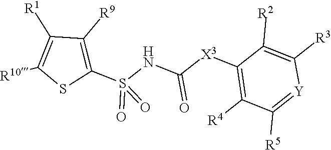

or a pharmaceutically acceptable salt thereof, and wherein:

X³ is NH or O;

or when X³ is NH, X³ and R² taken together with the atoms connecting them form a four-to-seven-membered heterocyclic ring optionally substituted with one or more R¹⁶;

or when X³ is NH, X³ and R⁴ taken together with the atoms connecting them form a four-to-seven-membered heterocyclic ring optionally substituted with one or more R¹⁶;

Y is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, C₁-C₆ haloalkoxy, and C₁-C₆ haloalkyl;

R⁹ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R³ is hydrogen, CN, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

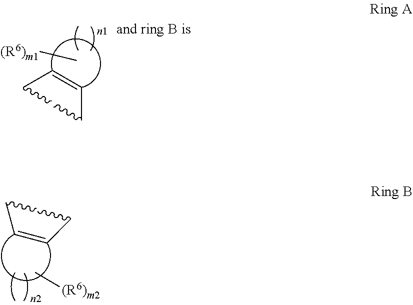

wherein

ring A is a saturated carbocyclic ring;

n1 is from 2 to 5;

m1 is from 1 to 10;

wherein ring B is a saturated carbocyclic ring;

n2 is from 2 to 5;

m2 is from 1 to 10;

wherein each R⁶ in each ring is the same or different and is selected from H, C₁-C₆ alkyl, C₁-C₆ alkoxy NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from H, unsubstituted C₁-C₆ alkyl, C(R¹⁹)₂OH, C(O)C₂-C₆ alkyl, and C₃-C₆ cycloalkyl;

wherein each C(O)C₂-C₆ alkyl and C₃-C₆ cycloalkyl above is optionally substituted with one or more substituents each independently selected from hydroxy, C₁-C₆ alkoxy, R¹¹R¹², ═NR¹³, COOC₁-C₆ alkyl, and CONR¹¹R¹²;

R^{10′″} is selected from C₁, C₁-C₆ alkyl substituted with hydroxy, C₃-C₆ cycloalkyl, and C₃-C₆ heterocycloalkyl;

wherein the C₃-C₆ cycloalkyl, and C₃-C₆ heterocycloalkyl above are each optionally substituted with one or more substituents each independently selected from hydroxy, oxo, C₁-C₆ alkoxy, NR¹¹R¹², ═NR¹³, COOC₁-C₆ alkyl, and CONR¹¹R¹²;

R¹³ is C₁-C₆ alkyl;

each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl;

each R¹⁶ is the same or different and is selected from H, C₁-C₆ alkyl, C₁-C₆ alkoxy, NR¹¹R¹², oxo, and ═NR¹³;

each R¹⁹ is the same and is selected from C₁-C₆ alkyl;

provided that if (1) R¹ is C(R¹⁹)₂OH; and (2) R^{10′″} is not C₁; then

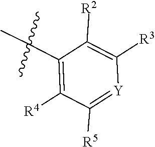

is not

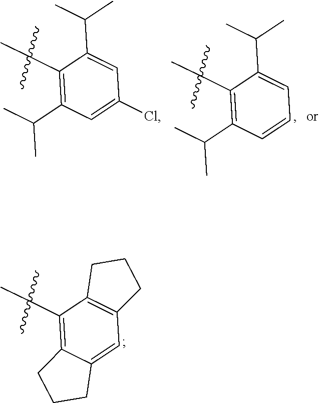

and provided that if R^{10′″} is C₁-C₆ alkyl substituted with hydroxy; then

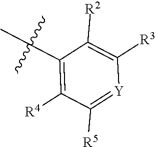

is not

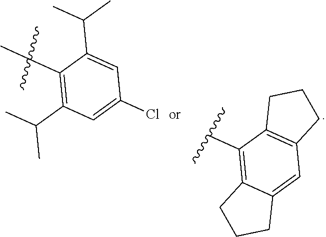 [SMILES: *c1c(C(C)C)cc(Cl)cc1C(C)C ; *c1c2c(cc3c1CCC3)CCC2]

In some embodiments of the compound of Formula II, R¹ is C(R¹⁹)₂OH.

In some embodiments of the compound of Formula II, R¹ is H.

In some embodiments of the compound of Formula II, R^{10′″} is C₁-C₆ alkyl substituted with hydroxyl.

In some embodiments of the compound of Formula II, R^{10′″} is 1-hydroxy-1-cyclopropyl.

In some embodiments of the compound of Formula II, R^{10′″} is C₁.

In some embodiments of the compound of Formula II, the compound of Formula II is

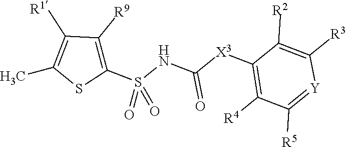

or a pharmaceutically acceptable salt thereof, and wherein:

X³ is NH or O;

or when X³ is NH, X³ and R² taken together with the atoms connecting them form a four-to-seven-membered heterocyclic ring optionally substituted with one or more R¹⁶;

or when X³ is NH, X³ and R⁴ taken together with the atoms connecting them form a four-to-seven-membered heterocyclic ring optionally substituted with one or more R¹⁶;

Y is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, C₁-C₆ haloalkoxy, and C₁-C₆ haloalkyl;

R⁹ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R³ is hydrogen, CN, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

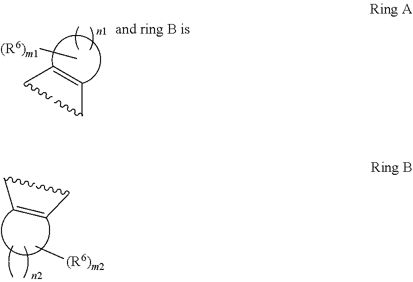

wherein

ring A is a saturated carbocyclic ring;

n1 is from 2 to 5;

m1 is from 1 to 10;

wherein ring B is a saturated carbocyclic ring;

n2 is from 2 to 5;

m2 is from 1 to 10;

wherein each R⁶ in each ring is the same or different and is selected from H, C₁-C₆ alkyl, C₁-C₆ alkoxy NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R^{1′} is selected from unsubstituted C₁-C₆ alkyl, C(R¹⁹)₂OH, C(O)C₂-C₆ alkyl, and C₃-C₆ cycloalkyl;

wherein each C(O)C₂-C₆ alkyl and C₃-C₆ cycloalkyl above is optionally substituted with one or more substituents each independently selected from hydroxy, C₁-C₆ alkoxy, NR¹¹R¹², ═NR¹³, COOC₁-C₆ alkyl, and CONR¹¹R¹²;

R¹³ is C₁-C₆ alkyl;

each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl;

each R¹⁶ is the same or different and is selected from H, C₁-C₆ alkyl, C₁-C₆ alkoxy, NR¹¹R¹², oxo, and ═NR¹³;

each R¹⁹ is the same and is selected from C₁-C₆ alkyl.

In some embodiments of the compound of Formula II, R^{1′} is C(R¹⁹)₂OH.

In some embodiments of the compound of Formula II, the compound of Formula II is

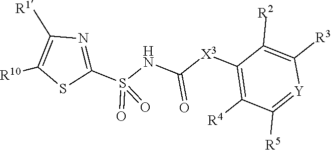

or a pharmaceutically acceptable salt thereof, and wherein:

X³ is NH or O;

or when X³ is NH, X³ and R² taken together with the atoms connecting them form a four-to-seven-membered heterocyclic ring optionally substituted with one or more R¹⁶;

or when X³ is NH, X³ and R⁴ taken together with the atoms connecting them form a four-to-seven-membered heterocyclic ring optionally substituted with one or more R¹⁶;

Y is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, C₁-C₆ haloalkoxy, and C₁-C₆ haloalkyl;

R⁹ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R³ is hydrogen, CN, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

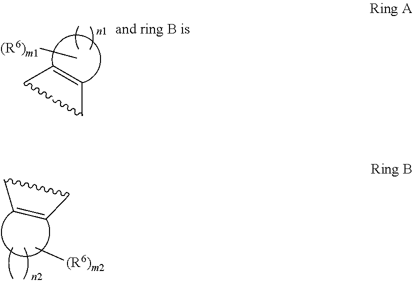

wherein

ring A is a saturated carbocyclic ring;

n1 is from 2 to 5;

m1 is from 1 to 10;

wherein ring B is a saturated carbocyclic ring;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R^{1′} is selected from unsubstituted C₁-C₆ alkyl, C(R¹⁹)₂OH, C(O)C₂-C₆ alkyl, and C₃-C₆ cycloalkyl;

wherein each C(O)C₂-C₆ alkyl and C₃-C₆ cycloalkyl above is optionally substituted with one or more substituents each independently selected from hydroxy, C₁-C₆ alkoxy, NR¹¹R¹², ═NR¹³, COOC₁-C₆ alkyl, and CONR¹¹R¹²;

R¹⁰ is selected from H, Cl, C₁-C₆ alkyl, C₃-C₆ cycloalkyl and C₃-C₆ heterocycloalkyl;

wherein R¹⁰ is optionally substituted with one or more substituents each independently selected from hydroxy, C₁-C₆ alkoxy, ═NR¹³, COOC₁-C₆ alkyl, and CONR¹¹R¹²;

R¹³ is C₁-C₆ alkyl;

each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl;

each R¹⁶ is the same or different and is selected from H, C₁-C₆ alkyl, C₁-C₆ alkoxy, NR¹¹R¹², oxo, and ═NR¹³;

each R¹⁹ is the same and is selected from C₁-C₆ alkyl.

In some embodiments of the compound of Formula II, R^{1′} is C(R¹⁹)₂OH and R¹⁰ is H.

In some embodiments of the compound of Formula II, the compound of Formula II is

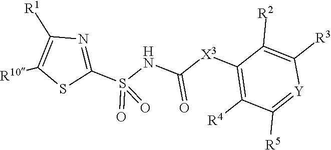

or a pharmaceutically acceptable salt thereof, and wherein:

X³ is NH or O;

or when X³ is NH, X³ and R² taken together with the atoms connecting them form a four-to-seven-membered heterocyclic ring optionally substituted with one or more R¹⁶;

or when X³ is NH, X³ and R⁴ taken together with the atoms connecting them form a four-to-seven-membered heterocyclic ring optionally substituted with one or more R¹⁶;

Y is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, C₁-C₆ haloalkoxy, and C₁-C₆ haloalkyl;

R⁹ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R³ is hydrogen, CN, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

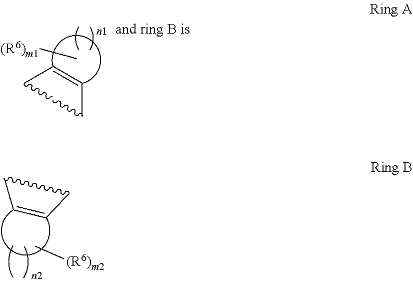

wherein

ring A is a saturated carbocyclic ring;

n1 is from 2 to 5;

m1 is from 1 to 10;

wherein ring B is a saturated carbocyclic ring;

n2 is from 2 to 5;

m2 is from 1 to 10;

wherein each R⁶ in each ring is the same or different and is selected from H, C₁-C₆ alkyl, C₁-C₆ alkoxy NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from H, unsubstituted C₁-C₆ alkyl, C(R¹⁹)₂OH, C(O)C₂-C₆ alkyl, and C₃-C₆ cycloalkyl;

wherein each C(O)C₂-C₆ alkyl and C₃-C₆ cycloalkyl above is optionally substituted with one or more substituents each independently selected from hydroxy, C₁-C₆ alkoxy,

NR¹¹R¹², ═NR¹³, COOC₁-C₆ alkyl, and CONR¹¹R¹²;

R^{10″} is selected from C₁, C₁-C₆ alkyl, C₃-C₆ cycloalkyl, and C₃-C₆ heterocycloalkyl;

wherein R^{10″} is optionally substituted with one or more substituents each independently selected from hydroxy, C₁-C₆ alkoxy, NR¹¹R¹², ═NR¹³, COOC₁-C₆ alkyl, and CONR¹¹R¹²;

R¹³ is C₁-C₆ alkyl;

each of R¹¹ and R¹² at each occurrence is independently selected from hydrogen,

C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl;

each R¹⁶ is the same or different and is selected from H, C₁-C₆ alkyl, C₁-C₆ alkoxy, NR¹¹R¹², oxo, and ═NR¹³;

each R¹⁹ is the same and is selected from C₁-C₆ alkyl.

In some embodiments of the compound of Formula II, R¹ is H and R^{10″} is C₃-C₆ cycloalkyl substituted with hydroxy.

In some embodiments of the compound of Formula II, the compound of Formula II is

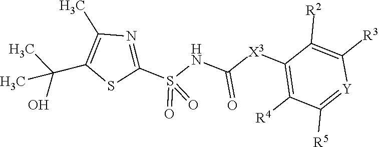

or a pharmaceutically acceptable salt thereof, and wherein:

X³ is NH or O;

or when X³ is NH, X³ and R² taken together with the atoms connecting them form a four-to-seven-membered heterocyclic ring optionally substituted with one or more R¹⁶;

or when X³ is NH, X³ and R⁴ taken together with the atoms connecting them form a four-to-seven-membered heterocyclic ring optionally substituted with one or more R¹⁶;

Y is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, C₁-C₆ haloalkoxy, and C₁-C₆ haloalkyl;

R⁹ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R³ is hydrogen, CN, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

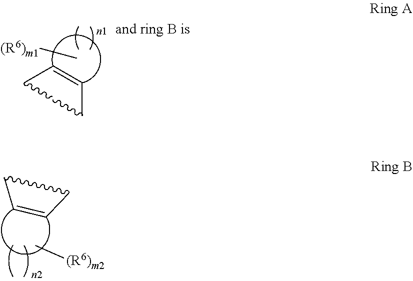

wherein

ring A is a saturated carbocyclic ring;

n1 is from 2 to 5;

m1 is from 1 to 10;

wherein ring B is a saturated carbocyclic ring;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹³ is C₁-C₆ alkyl;

each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl;

each R¹⁶ is the same or different and is selected from H, C₁-C₆ alkyl, C₁-C₆ alkoxy, NR¹¹R¹², oxo, and ═NR¹³.

In some embodiments of the compound of Formula II, the compound of Formula II is

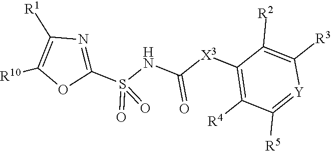

or a pharmaceutically acceptable salt thereof, and wherein:

X³ is NH or O;

or when X³ is NH, X³ and R² taken together with the atoms connecting them form a four-to-seven-membered heterocyclic ring optionally substituted with one or more R¹⁶;

or when X³ is NH, X³ and R⁴ taken together with the atoms connecting them form a four-to-seven-membered heterocyclic ring optionally substituted with one or more R¹⁶;

Y is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, C₁-C₆ haloalkoxy, and C₁-C₆ haloalkyl;

R⁹ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R³ is hydrogen, CN, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

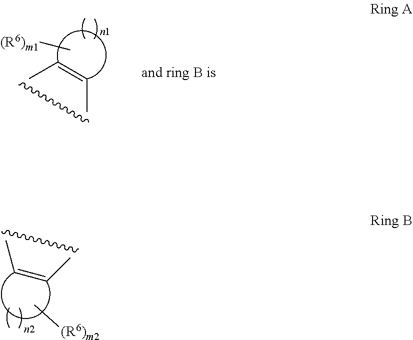

wherein

ring A is a saturated carbocyclic ring;

n1 is from 2 to 5;

m1 is from 1 to 10;

wherein ring B is a saturated carbocyclic ring;

n2 is from 2 to 5;

m2 is from 1 to 10;

wherein each R⁶ in each ring is the same or different and is selected from H, C₁-C₆ alkyl, C₁-C₆ alkoxy NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from H, unsubstituted C₁-C₆ alkyl, C(R¹⁹)₂OH, C(O)C₂-C₆ alkyl, and C₃-C₆ cycloalkyl;

wherein each C(O)C₂-C₆ alkyl and C₃-C₆ cycloalkyl above is optionally substituted with one or more substituents each independently selected from hydroxy, C₁-C₆ alkoxy, NR¹¹R¹², —NR¹³, COOC₁-C₆ alkyl, and CONR¹¹R¹²;

R¹⁰ is selected from H, Cl, C₁-C₆ alkyl, C₃-C₆ cycloalkyl and C₃-C₆ heterocycloalkyl;

wherein R¹⁰ is optionally substituted with one or more substituents each independently selected from hydroxy, C₁-C₆ alkoxy, NR¹¹R¹², —NR¹³, COOC₁-C₆ alkyl, and CONR¹¹R¹²;

or R¹ and R¹⁰ taken together with the atoms connecting them form a 3-to-8-membered carbocyclic or heterocyclic ring containing 1 or 2 heteroatoms independently selected from O, N, and S, wherein the ring is optionally substituted with one or more substituents each independently selected from hydroxy, oxo, C₁-C₆ alkoxy, NR¹¹R¹², ═NR¹³, COOC₁-C₆ alkyl, and CONR¹¹R¹²;

R¹³ is C₁-C₆ alkyl;

each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl;

each R¹⁶ is the same or different and is selected from H, C₁-C₆ alkyl, C₁-C₆ alkoxy, NR¹¹R¹², oxo, and ═NR¹³;

each R¹⁹ is the same and is selected from C₁-C₆ alkyl.

In some embodiments of the compound of Formula II, R¹ is H and R¹⁰ is C₁-C₆ alkyl substituted with hydroxy.

In some embodiments of the compound of Formula II, R¹ is C(R¹⁹)₂OH and R¹⁰ is H.

In some embodiments of the compound of Formula II, the compound of Formula II is

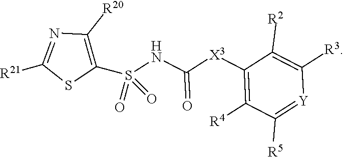

In some embodiments of the compound of Formula II, R²⁰ is H and R²¹ is C₁-C₆ alkyl substituted with hydroxyl.

In some embodiments of the compound of Formula II, R²¹ is H and R²⁰ is C₁-C₆ alkyl substituted with hydroxyl.

In some embodiments of the compound of Formula II, R⁹ is H.

In some embodiments of the compound of Formula II, X³ is NH.

In some embodiments of the compound of Formula II, each R¹⁹ is methyl.

In some embodiments of the compound of Formula II, R² and R³ taken together with the carbons connecting them form a five membered carbocyclic ring and R⁴ and R⁵ taken together with the carbons connecting them form a five membered carbocyclic ring.

In some embodiments of the compound of Formula II, R² and R⁴ are each isopropyl.

In some embodiments of the compound of Formula II, R³ and R⁵ are each H.

In some embodiments of the compound of Formula II, R³ is halo and R⁵ is H.

In some embodiments of the compound of Formula II, R³ is CN and R⁵ is H.

In some embodiments of the compound of Formula II, R⁸ is H.

In some embodiments of the compound of Formula II, R⁸ is F.

In some embodiments of the compound of Formula II, R⁸ is C₁.

In some embodiments of the compound of Formula II, R⁸ is CN.

In another aspect, an NLRP3 antagonist is a compound selected from the group consisting of compounds in the Table 1 below and pharmaceutically acceptable salts thereof.

TABLE 1

| Com- | |
|---|---|
| pound | Structure |
| 101 | 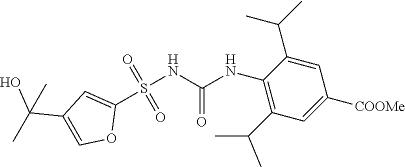 [SMILES: COC(=O)c1cc(C(C)C)c(NC(=O)NS(=O)(=O)c2cc(C(C)(C)O)co2)c(C(C)C)c1] |
| 102 | 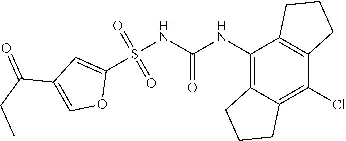 [SMILES: CCC(=O)c1coc(S(=O)(=O)NC(=O)Nc2c3c(c(Cl)c4c2CCC4)CCC3)c1] |
| 103 | 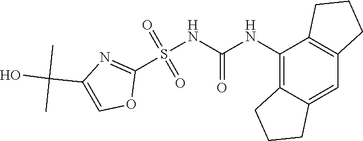 [SMILES: CC(C)(O)c1coc(S(=O)(=O)NC(=O)Nc2c3c(cc4c2CCC4)CCC3)n1] |
| 104 | 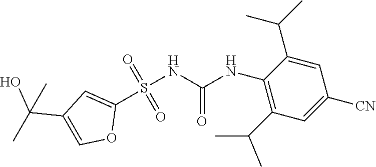 [SMILES: CC(C)c1cc(C#N)cc(C(C)C)c1NC(=O)NS(=O)(=O)c1cc(C(C)(C)O)co1] |
| 105 | 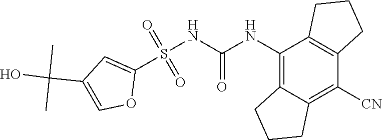 [SMILES: CC(C)(O)c1coc(S(=O)(=O)NC(=O)Nc2c3c(c(C#N)c4c2CCC4)CCC3)c1] |
| 106 | 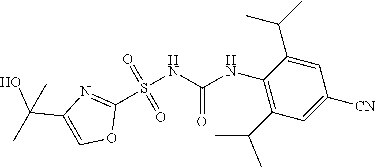 [SMILES: CC(C)c1cc(C#N)cc(C(C)C)c1NC(=O)NS(=O)(=O)c1nc(C(C)(C)O)co1] |
| 108 | 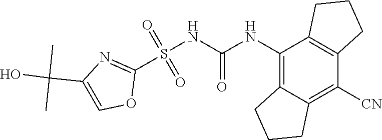 [SMILES: CC(C)(O)c1coc(S(=O)(=O)NC(=O)Nc2c3c(c(C#N)c4c2CCC4)CCC3)n1] |
| 109 | 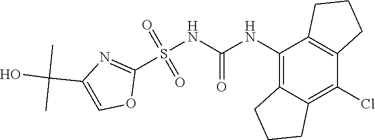 [SMILES: CC(C)(O)c1coc(S(=O)(=O)NC(=O)Nc2c3c(c(Cl)c4c2CCC4)CCC3)n1] |
| 110 | 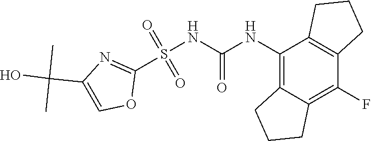 [SMILES: CC(C)(O)c1coc(S(=O)(=O)NC(=O)Nc2c3c(c(F)c4c2CCC4)CCC3)n1] |
| 111 | 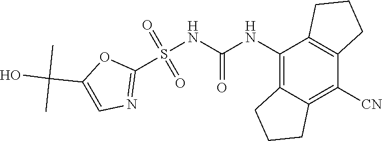 [SMILES: CC(C)(O)c1cnc(S(=O)(=O)NC(=O)Nc2c3c(c(C#N)c4c2CCC4)CCC3)o1] |
| 112 | 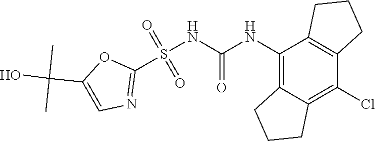 [SMILES: CC(C)(O)c1cnc(S(=O)(=O)NC(=O)Nc2c3c(c(Cl)c4c2CCC4)CCC3)o1] |
| 113 | 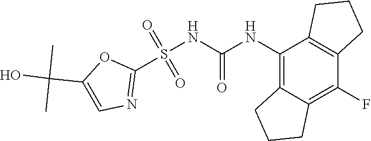 [SMILES: CC(C)(O)c1cnc(S(=O)(=O)NC(=O)Nc2c3c(c(F)c4c2CCC4)CCC3)o1] |
| 114 | 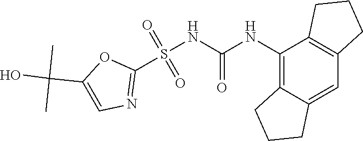 [SMILES: CC(C)(O)c1cnc(S(=O)(=O)NC(=O)Nc2c3c(cc4c2CCC4)CCC3)o1] |
| 115 | 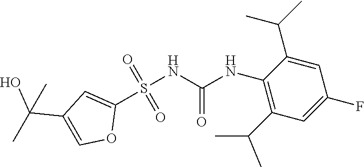 [SMILES: CC(C)c1cc(F)cc(C(C)C)c1NC(=O)NS(=O)(=O)c1cc(C(C)(C)O)co1] |
| 116 | 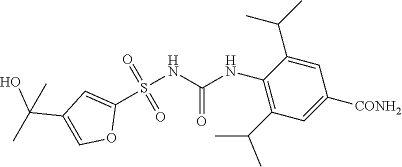 [SMILES: CC(C)c1cc(C(N)=O)cc(C(C)C)c1NC(=O)NS(=O)(=O)c1cc(C(C)(C)O)co1] |
| 117 | 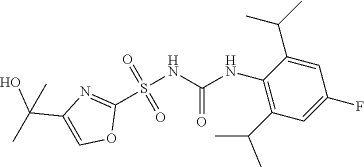 [SMILES: CC(C)c1cc(F)cc(C(C)C)c1NC(=O)NS(=O)(=O)c1nc(C(C)(C)O)co1] |
| 118 | 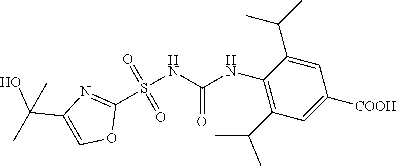 [SMILES: CC(C)c1cc(C(=O)O)cc(C(C)C)c1NC(=O)NS(=O)(=O)c1nc(C(C)(C)O)co1] |
| 119 | 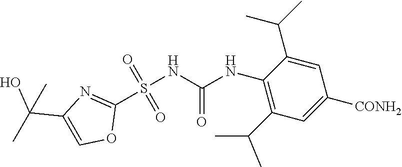 [SMILES: CC(C)c1cc(C(N)=O)cc(C(C)C)c1NC(=O)NS(=O)(=O)c1nc(C(C)(C)O)co1] |
| 120 | 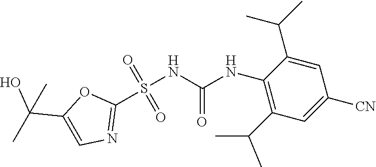 [SMILES: CC(C)c1cc(C#N)cc(C(C)C)c1NC(=O)NS(=O)(=O)c1ncc(C(C)(C)O)o1] |
| 121 | 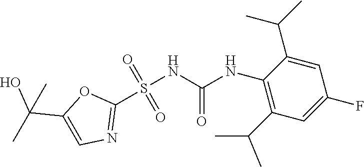 [SMILES: CC(C)c1cc(F)cc(C(C)C)c1NC(=O)NS(=O)(=O)c1ncc(C(C)(C)O)o1] |
| 122 | 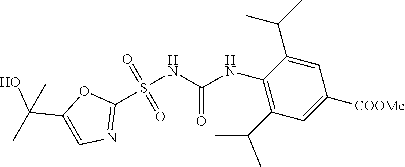 [SMILES: COC(=O)c1cc(C(C)C)c(NC(=O)NS(=O)(=O)c2ncc(C(C)(C)O)o2)c(C(C)C)c1] |
| 123 | 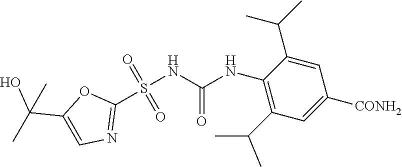 [SMILES: CC(C)c1cc(C(N)=O)cc(C(C)C)c1NC(=O)NS(=O)(=O)c1ncc(C(C)(C)O)o1] |
| 124 | 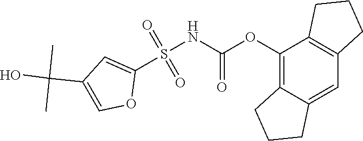 [SMILES: CC(C)(O)c1coc(S(=O)(=O)NC(=O)Oc2c3c(cc4c2CCC4)CCC3)c1] |
| 126 | 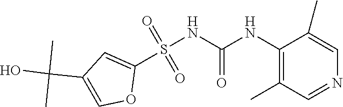 [SMILES: Cc1cncc(C)c1NC(=O)NS(=O)(=O)c1cc(C(C)(C)O)co1] |
| 127 | 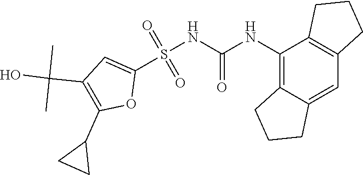 [SMILES: CC(C)(O)c1cc(S(=O)(=O)NC(=O)Nc2c3c(cc4c2CCC4)CCC3)oc1C1CC1] |
| 128 | 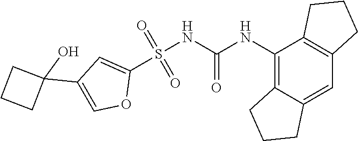 [SMILES: O=C(Nc1c2c(cc3c1CCC3)CCC2)NS(=O)(=O)c1cc(C2(O)CCC2)co1] |
| 129 | 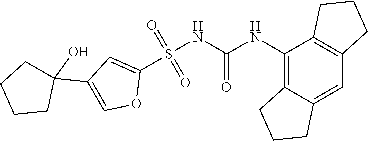 [SMILES: O=C(Nc1c2c(cc3c1CCC3)CCC2)NS(=O)(=O)c1cc(C2(O)CCCC2)co1] |
| 130 | 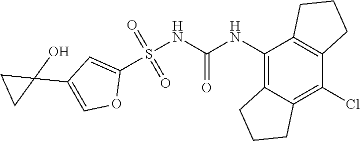 [SMILES: O=C(Nc1c2c(c(Cl)c3c1CCC3)CCC2)NS(=O)(=O)c1cc(C2(O)CC2)co1] |
| 132 | 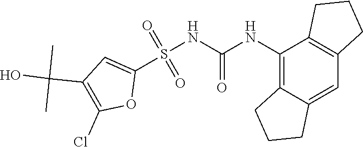 [SMILES: CC(C)(O)c1cc(S(=O)(=O)NC(=O)Nc2c3c(cc4c2CCC4)CCC3)oc1Cl] |
| 135 | 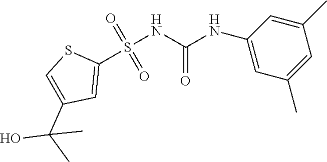 [SMILES: Cc1cc(C)cc(NC(=O)NS(=O)(=O)c2cc(C(C)(C)O)cs2)c1] |
| 136 | 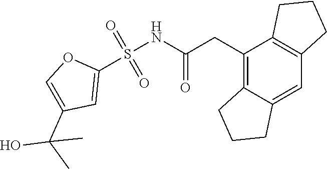 [SMILES: CC(C)(O)c1coc(S(=O)(=O)NC(=O)Cc2c3c(cc4c2CCC4)CCC3)c1] |
| 137 | 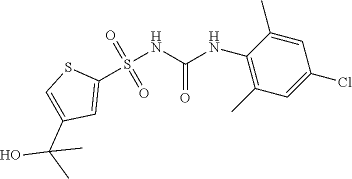 [SMILES: Cc1cc(Cl)cc(C)c1NC(=O)NS(=O)(=O)c1cc(C(C)(C)O)cs1] |
| 138a | 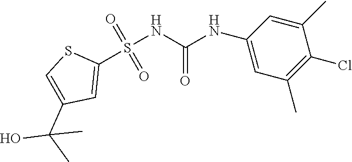 [SMILES: Cc1cc(NC(=O)NS(=O)(=O)c2cc(C(C)(C)O)cs2)cc(C)c1Cl] |

In another aspect, an NLRP3 antagonist is a compound selected from the group consisting of compounds in the Table 2 below and pharmaceutically acceptable salts thereof.

TABLE 2

| Compound | Structure |
|---|---|
| 138 | 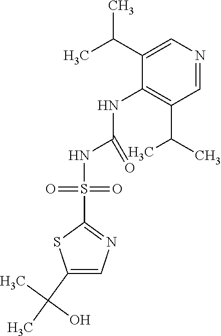 [SMILES: CC(C)c1cncc(C(C)C)c1NC(=O)NS(=O)(=O)c1ncc(C(C)(C)O)s1] |
| 139 | 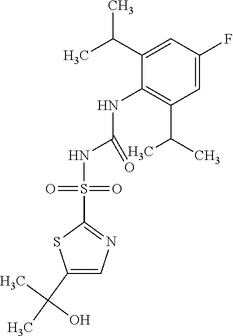 [SMILES: CC(C)c1cc(F)cc(C(C)C)c1NC(=O)NS(=O)(=O)c1ncc(C(C)(C)O)s1] |
| 142 | 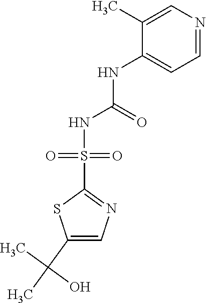 [SMILES: Cc1cnccc1NC(=O)NS(=O)(=O)c1ncc(C(C)(C)O)s1] |
| 143 | 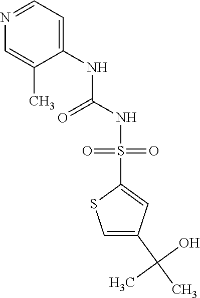 [SMILES: Cc1cnccc1NC(=O)NS(=O)(=O)c1cc(C(C)(C)O)cs1] |
| 144 | 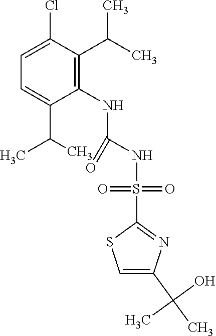 [SMILES: CC(C)c1ccc(Cl)c(C(C)C)c1NC(=O)NS(=O)(=O)c1nc(C(C)(C)O)cs1] |
| 145 | 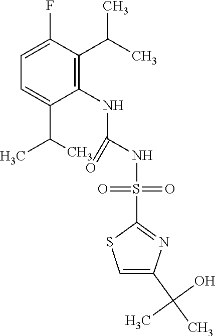 [SMILES: CC(C)c1ccc(F)c(C(C)C)c1NC(=O)NS(=O)(=O)c1nc(C(C)(C)O)cs1] |
| 146 | 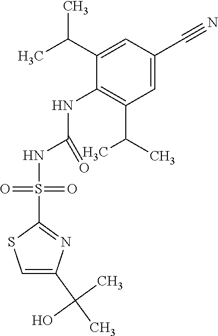 [SMILES: CC(C)c1cc(C#N)cc(C(C)C)c1NC(=O)NS(=O)(=O)c1nc(C(C)(C)O)cs1] |
| 147 | 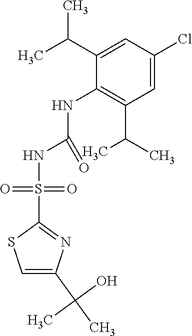 [SMILES: CC(C)c1cc(Cl)cc(C(C)C)c1NC(=O)NS(=O)(=O)c1nc(C(C)(C)O)cs1] |
| 148 | 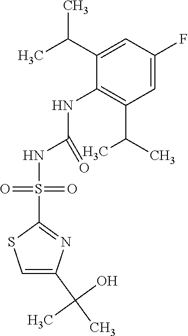 [SMILES: CC(C)c1cc(F)cc(C(C)C)c1NC(=O)NS(=O)(=O)c1nc(C(C)(C)O)cs1] |
| 149 | 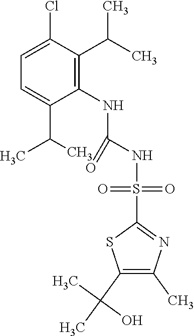 [SMILES: Cc1nc(S(=O)(=O)NC(=O)Nc2c(C(C)C)ccc(Cl)c2C(C)C)sc1C(C)(C)O] |
| 150 | 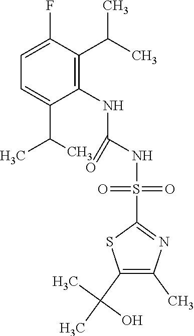 [SMILES: Cc1nc(S(=O)(=O)NC(=O)Nc2c(C(C)C)ccc(F)c2C(C)C)sc1C(C)(C)O] |
| 151 | 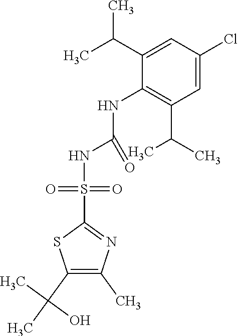 [SMILES: Cc1nc(S(=O)(=O)NC(=O)Nc2c(C(C)C)cc(Cl)cc2C(C)C)sc1C(C)(C)O] |
| 152 | 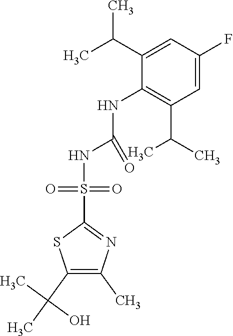 [SMILES: Cc1nc(S(=O)(=O)NC(=O)Nc2c(C(C)C)cc(F)cc2C(C)C)sc1C(C)(C)O] |
| 153 | 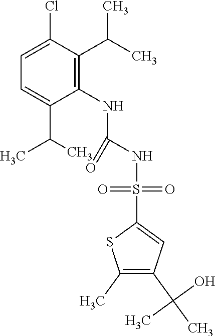 [SMILES: Cc1sc(S(=O)(=O)NC(=O)Nc2c(C(C)C)ccc(Cl)c2C(C)C)cc1C(C)(C)O] |
| 154 | 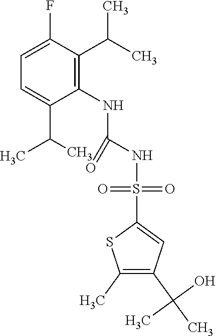 [SMILES: Cc1sc(S(=O)(=O)NC(=O)Nc2c(C(C)C)ccc(F)c2C(C)C)cc1C(C)(C)O] |
| 155 | 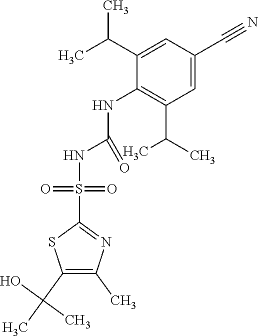 [SMILES: Cc1nc(S(=O)(=O)NC(=O)Nc2c(C(C)C)cc(C#N)cc2C(C)C)sc1C(C)(C)O] |
| 156 | 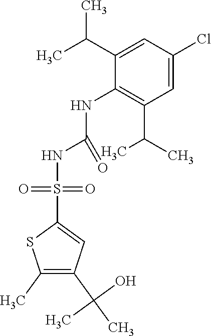 [SMILES: Cc1sc(S(=O)(=O)NC(=O)Nc2c(C(C)C)cc(Cl)cc2C(C)C)cc1C(C)(C)O] |
| 157 | 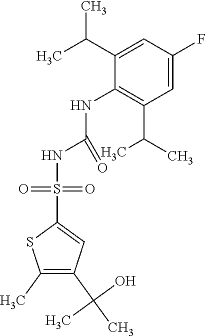 [SMILES: Cc1sc(S(=O)(=O)NC(=O)Nc2c(C(C)C)cc(F)cc2C(C)C)cc1C(C)(C)O] |
| 158 | 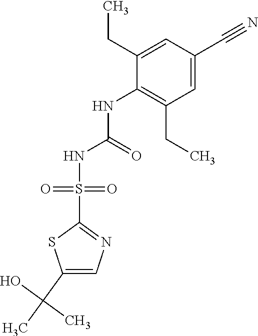 [SMILES: CCc1cc(C#N)cc(CC)c1NC(=O)NS(=O)(=O)c1ncc(C(C)(C)O)s1] |
| 159 | 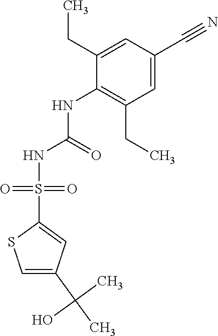 [SMILES: CCc1cc(C#N)cc(CC)c1NC(=O)NS(=O)(=O)c1cc(C(C)(C)O)cs1] |
| 160 | 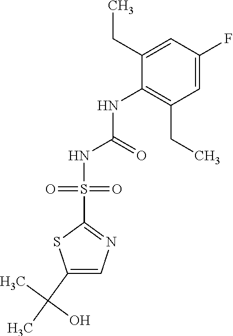 [SMILES: CCc1cc(F)cc(CC)c1NC(=O)NS(=O)(=O)c1ncc(C(C)(C)O)s1] |
| 161 | 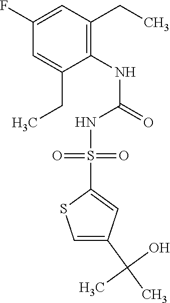 [SMILES: CCc1cc(F)cc(CC)c1NC(=O)NS(=O)(=O)c1cc(C(C)(C)O)cs1] |
| 162 | 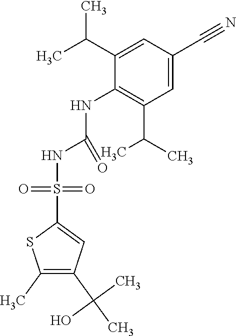 [SMILES: Cc1sc(S(=O)(=O)NC(=O)Nc2c(C(C)C)cc(C#N)cc2C(C)C)cc1C(C)(C)O] |
| 163 | 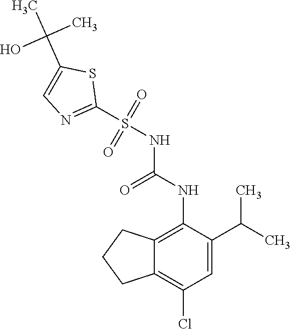 [SMILES: CC(C)c1cc(Cl)c2c(c1NC(=O)NS(=O)(=O)c1ncc(C(C)(C)O)s1)CCC2] |
| 164 | 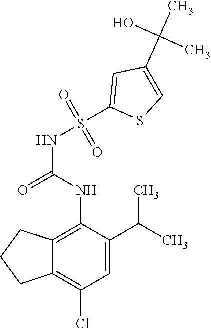 [SMILES: CC(C)c1cc(Cl)c2c(c1NC(=O)NS(=O)(=O)c1cc(C(C)(C)O)cs1)CCC2] |
| 165 | 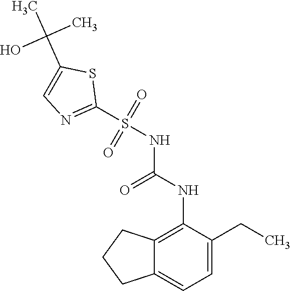 [SMILES: CCc1ccc2c(c1NC(=O)NS(=O)(=O)c1ncc(C(C)(C)O)s1)CCC2] |
| 166 | 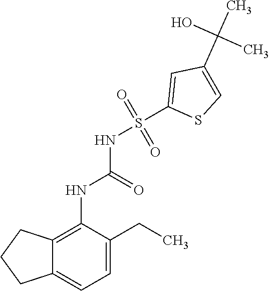 [SMILES: CCc1ccc2c(c1NC(=O)NS(=O)(=O)c1cc(C(C)(C)O)cs1)CCC2] |
| 167 | 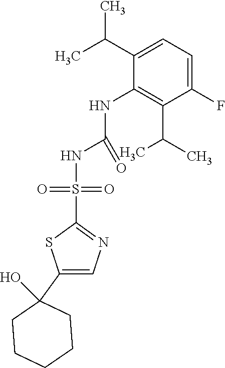 [SMILES: CC(C)c1ccc(F)c(C(C)C)c1NC(=O)NS(=O)(=O)c1ncc(C2(O)CCCCC2)s1] |
| 168 | 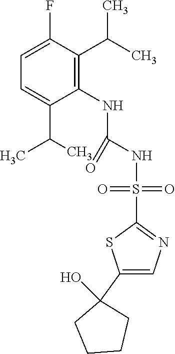 [SMILES: CC(C)c1ccc(F)c(C(C)C)c1NC(=O)NS(=O)(=O)c1ncc(C2(O)CCCC2)s1] |
| 169 | 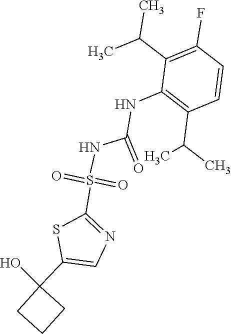 [SMILES: CC(C)c1ccc(F)c(C(C)C)c1NC(=O)NS(=O)(=O)c1ncc(C2(O)CCC2)s1] |
| 170 | 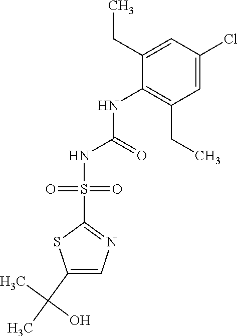 [SMILES: CCc1cc(Cl)cc(CC)c1NC(=O)NS(=O)(=O)c1ncc(C(C)(C)O)s1] |
| 171 | 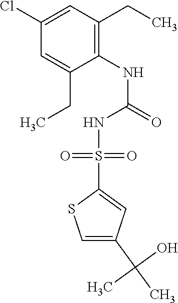 [SMILES: CCc1cc(Cl)cc(CC)c1NC(=O)NS(=O)(=O)c1cc(C(C)(C)O)cs1] |
| 172 | 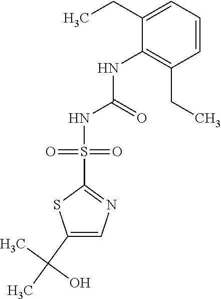 [SMILES: CCc1cccc(CC)c1NC(=O)NS(=O)(=O)c1ncc(C(C)(C)O)s1] |
| 173 | 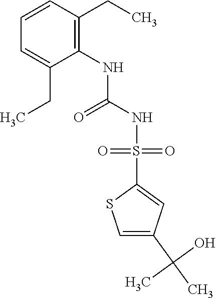 [SMILES: CCc1cccc(CC)c1NC(=O)NS(=O)(=O)c1cc(C(C)(C)O)cs1] |
| 175 | 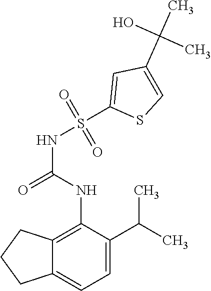 [SMILES: CC(C)c1ccc2c(c1NC(=O)NS(=O)(=O)c1cc(C(C)(C)O)cs1)CCC2] |
| 176 | 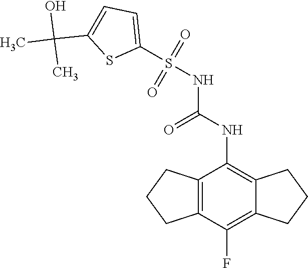 [SMILES: CC(C)(O)c1ccc(S(=O)(=O)NC(=O)Nc2c3c(c(F)c4c2CCC4)CCC3)s1] |
| 178 | 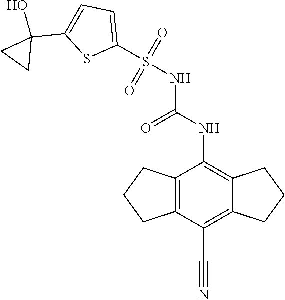 [SMILES: N#Cc1c2c(c(NC(=O)NS(=O)(=O)c3ccc(C4(O)CC4)s3)c3c1CCC3)CCC2] |
| 179 | 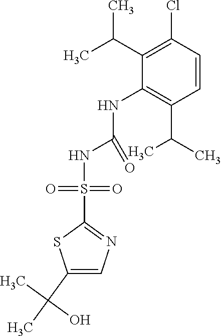 [SMILES: CC(C)c1ccc(Cl)c(C(C)C)c1NC(=O)NS(=O)(=O)c1ncc(C(C)(C)O)s1] |
| 180 | 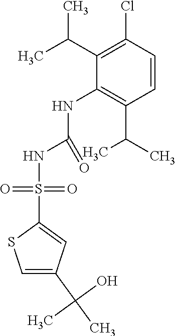 [SMILES: CC(C)c1ccc(Cl)c(C(C)C)c1NC(=O)NS(=O)(=O)c1cc(C(C)(C)O)cs1] |
| 181 | 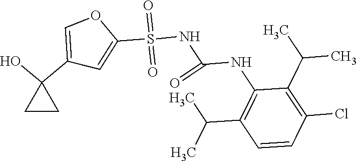 [SMILES: CC(C)c1ccc(Cl)c(C(C)C)c1NC(=O)NS(=O)(=O)c1cc(C2(O)CC2)co1] |
| 182 | 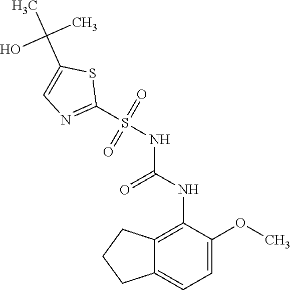 [SMILES: COc1ccc2c(c1NC(=O)NS(=O)(=O)c1ncc(C(C)(C)O)s1)CCC2] |
| 183 | 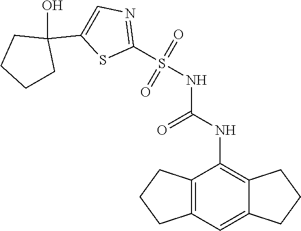 [SMILES: O=C(Nc1c2c(cc3c1CCC3)CCC2)NS(=O)(=O)c1ncc(C2(O)CCCC2)s1] |
| 184 | 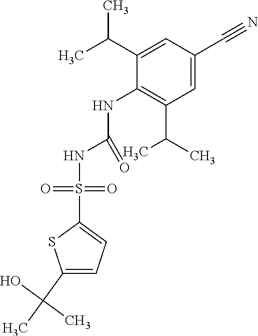 [SMILES: CC(C)c1cc(C#N)cc(C(C)C)c1NC(=O)NS(=O)(=O)c1ccc(C(C)(C)O)s1] |
| 185 | 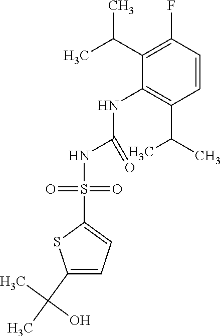 [SMILES: CC(C)c1ccc(F)c(C(C)C)c1NC(=O)NS(=O)(=O)c1ccc(C(C)(C)O)s1] |
| 187 | 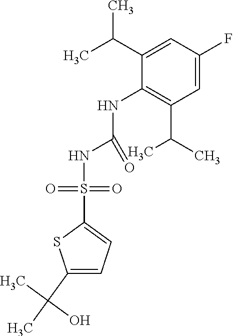 [SMILES: CC(C)c1cc(F)cc(C(C)C)c1NC(=O)NS(=O)(=O)c1ccc(C(C)(C)O)s1] |
| 188 | 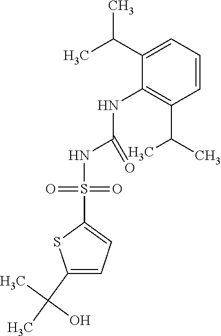 [SMILES: CC(C)c1cccc(C(C)C)c1NC(=O)NS(=O)(=O)c1ccc(C(C)(C)O)s1] |
| 189 | 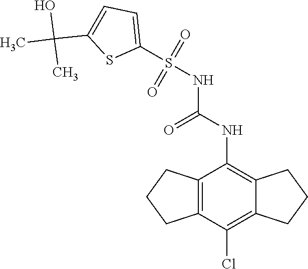 [SMILES: CC(C)(O)c1ccc(S(=O)(=O)NC(=O)Nc2c3c(c(Cl)c4c2CCC4)CCC3)s1] |
| 190 | 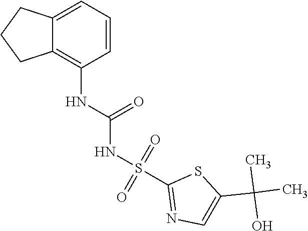 [SMILES: CC(C)(O)c1cnc(S(=O)(=O)NC(=O)Nc2cccc3c2CCC3)s1] |
| 191 | 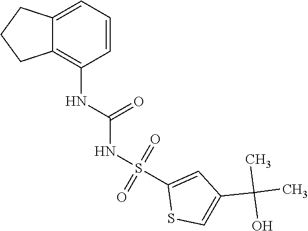 [SMILES: CC(C)(O)c1csc(S(=O)(=O)NC(=O)Nc2cccc3c2CCC3)c1] |
| 192 | 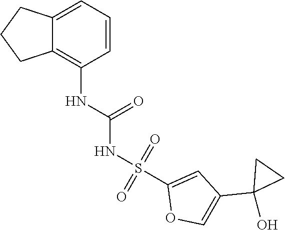 [SMILES: O=C(Nc1cccc2c1CCC2)NS(=O)(=O)c1cc(C2(O)CC2)co1] |
| 193 | 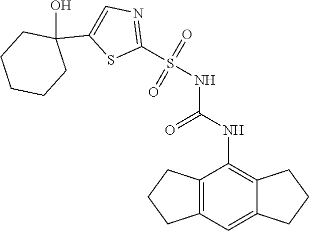 [SMILES: O=C(Nc1c2c(cc3c1CCC3)CCC2)NS(=O)(=O)c1ncc(C2(O)CCCCC2)s1] |
| 194 | 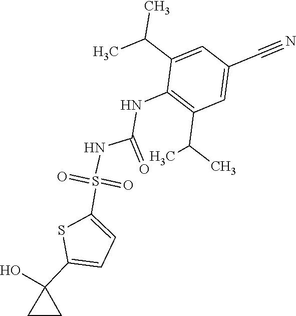 [SMILES: CC(C)c1cc(C#N)cc(C(C)C)c1NC(=O)NS(=O)(=O)c1ccc(C2(O)CC2)s1] |
| 195 | 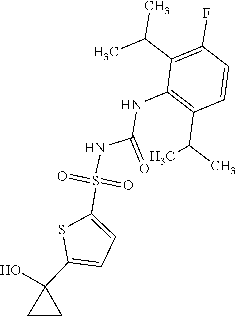 [SMILES: CC(C)c1ccc(F)c(C(C)C)c1NC(=O)NS(=O)(=O)c1ccc(C2(O)CC2)s1] |
| 196 | 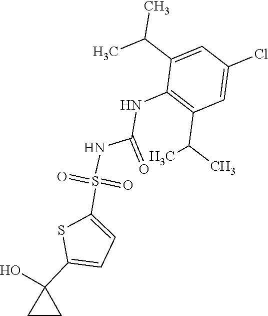 [SMILES: CC(C)c1cc(Cl)cc(C(C)C)c1NC(=O)NS(=O)(=O)c1ccc(C2(O)CC2)s1] |
| 197 | 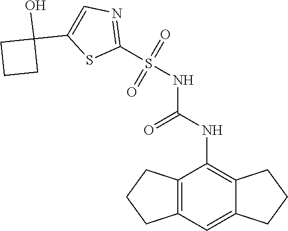 [SMILES: O=C(Nc1c2c(cc3c1CCC3)CCC2)NS(=O)(=O)c1ncc(C2(O)CCC2)s1] |
| 198 | 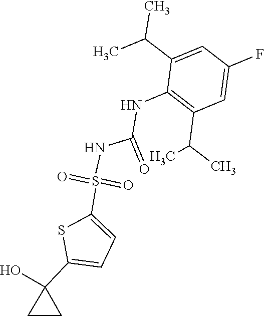 [SMILES: CC(C)c1cc(F)cc(C(C)C)c1NC(=O)NS(=O)(=O)c1ccc(C2(O)CC2)s1] |
| 199 | 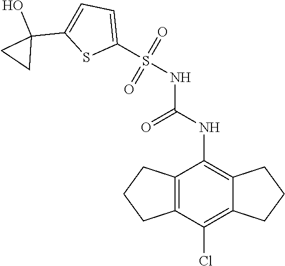 [SMILES: O=C(Nc1c2c(c(Cl)c3c1CCC3)CCC2)NS(=O)(=O)c1ccc(C2(O)CC2)s1] |
| 200 | 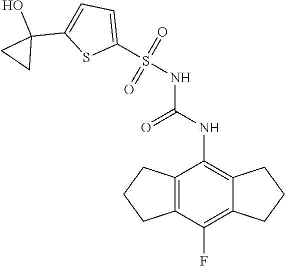 [SMILES: O=C(Nc1c2c(c(F)c3c1CCC3)CCC2)NS(=O)(=O)c1ccc(C2(O)CC2)s1] |
| 201 | 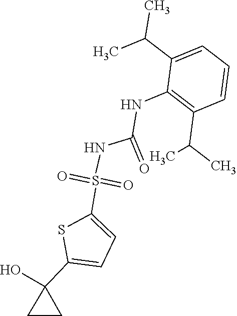 [SMILES: CC(C)c1cccc(C(C)C)c1NC(=O)NS(=O)(=O)c1ccc(C2(O)CC2)s1] |
| 202 | 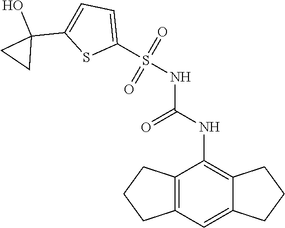 [SMILES: O=C(Nc1c2c(cc3c1CCC3)CCC2)NS(=O)(=O)c1ccc(C2(O)CC2)s1] |
| 204 | 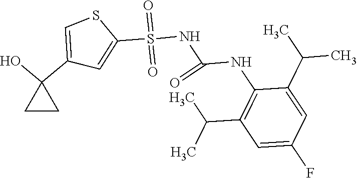 [SMILES: CC(C)c1cc(F)cc(C(C)C)c1NC(=O)NS(=O)(=O)c1cc(C2(O)CC2)cs1] |
| 205 | 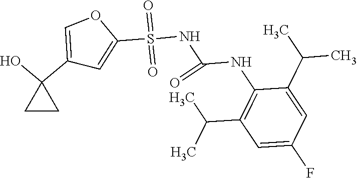 [SMILES: CC(C)c1cc(F)cc(C(C)C)c1NC(=O)NS(=O)(=O)c1cc(C2(O)CC2)co1] |
| 206 | 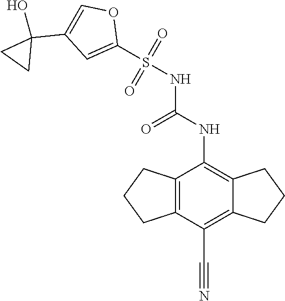 [SMILES: N#Cc1c2c(c(NC(=O)NS(=O)(=O)c3cc(C4(O)CC4)co3)c3c1CCC3)CCC2] |
| 207 | 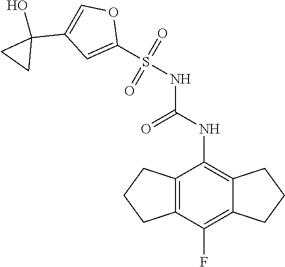 [SMILES: O=C(Nc1c2c(c(F)c3c1CCC3)CCC2)NS(=O)(=O)c1cc(C2(O)CC2)co1] |
| 209 | 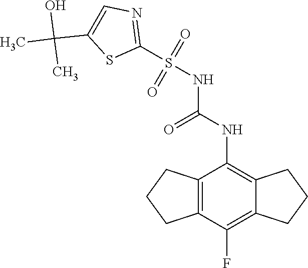 [SMILES: CC(C)(O)c1cnc(S(=O)(=O)NC(=O)Nc2c3c(c(F)c4c2CCC4)CCC3)s1] |
| 210 | 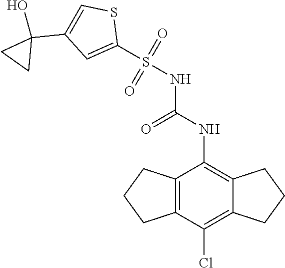 [SMILES: O=C(Nc1c2c(c(Cl)c3c1CCC3)CCC2)NS(=O)(=O)c1cc(C2(O)CC2)cs1] |
| 211 | 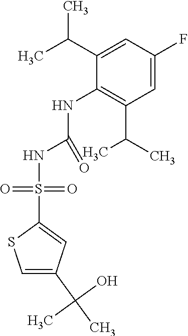 [SMILES: CC(C)c1cc(F)cc(C(C)C)c1NC(=O)NS(=O)(=O)c1cc(C(C)(C)O)cs1] |
| 212 | 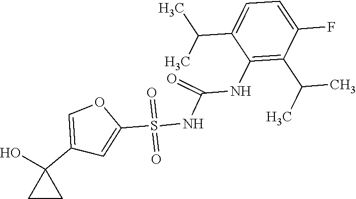 [SMILES: CC(C)c1ccc(F)c(C(C)C)c1NC(=O)NS(=O)(=O)c1cc(C2(O)CC2)co1] |
| 213 | 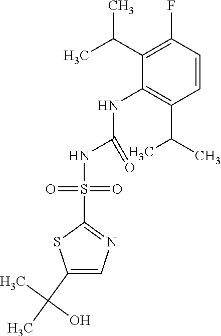 [SMILES: CC(C)c1ccc(F)c(C(C)C)c1NC(=O)NS(=O)(=O)c1ncc(C(C)(C)O)s1] |
| 214 | 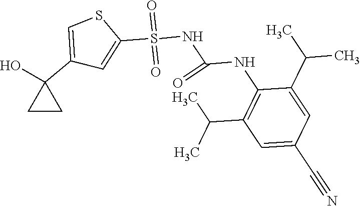 [SMILES: CC(C)c1cc(C#N)cc(C(C)C)c1NC(=O)NS(=O)(=O)c1cc(C2(O)CC2)cs1] |
| 215 | 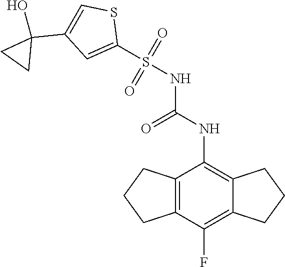 [SMILES: O=C(Nc1c2c(c(F)c3c1CCC3)CCC2)NS(=O)(=O)c1cc(C2(O)CC2)cs1] |
| 216 | 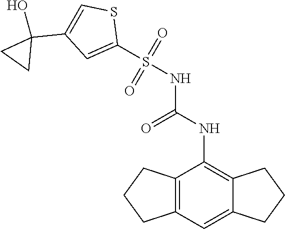 [SMILES: O=C(Nc1c2c(cc3c1CCC3)CCC2)NS(=O)(=O)c1cc(C2(O)CC2)cs1] |
| 217 | 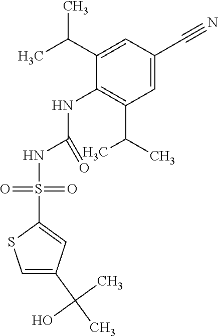 [SMILES: CC(C)c1cc(C#N)cc(C(C)C)c1NC(=O)NS(=O)(=O)c1cc(C(C)(C)O)cs1] |
| 218 | 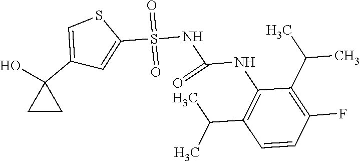 [SMILES: CC(C)c1ccc(F)c(C(C)C)c1NC(=O)NS(=O)(=O)c1cc(C2(O)CC2)cs1] |
| 219 | 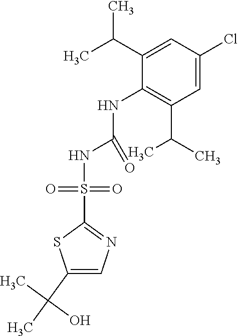 [SMILES: CC(C)c1cc(Cl)cc(C(C)C)c1NC(=O)NS(=O)(=O)c1ncc(C(C)(C)O)s1] |
| 220 | 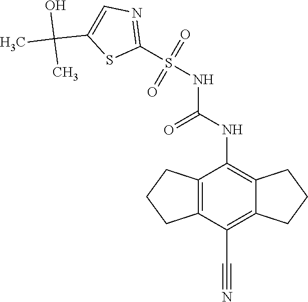 [SMILES: CC(C)(O)c1cnc(S(=O)(=O)NC(=O)Nc2c3c(c(C#N)c4c2CCC4)CCC3)s1] |
| 221 | 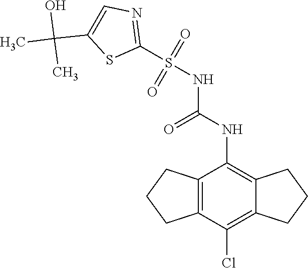 [SMILES: CC(C)(O)c1cnc(S(=O)(=O)NC(=O)Nc2c3c(c(Cl)c4c2CCC4)CCC3)s1] |
| 222 | 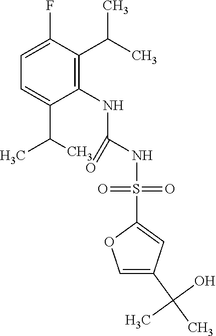 [SMILES: CC(C)c1ccc(F)c(C(C)C)c1NC(=O)NS(=O)(=O)c1cc(C(C)(C)O)co1] |
| 223 | 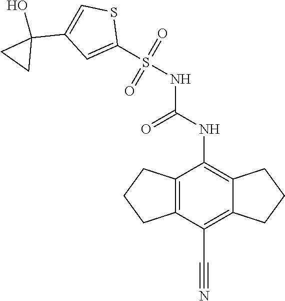 [SMILES: N#Cc1c2c(c(NC(=O)NS(=O)(=O)c3cc(C4(O)CC4)cs3)c3c1CCC3)CCC2] |
| 224 | 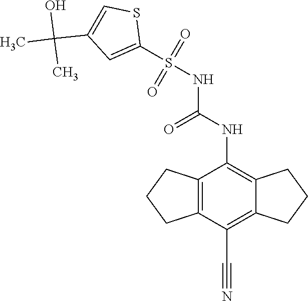 [SMILES: CC(C)(O)c1csc(S(=O)(=O)NC(=O)Nc2c3c(c(C#N)c4c2CCC4)CCC3)c1] |
| 225 | 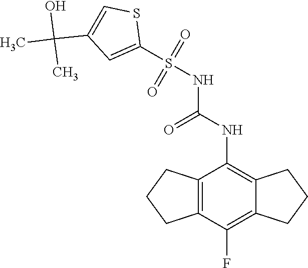 [SMILES: CC(C)(O)c1csc(S(=O)(=O)NC(=O)Nc2c3c(c(F)c4c2CCC4)CCC3)c1] |
| 226 | 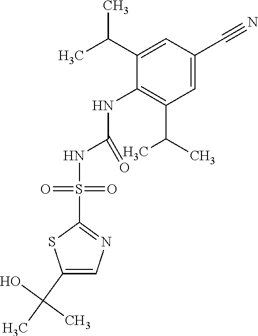 [SMILES: CC(C)c1cc(C#N)cc(C(C)C)c1NC(=O)NS(=O)(=O)c1ncc(C(C)(C)O)s1] |
| 227 | 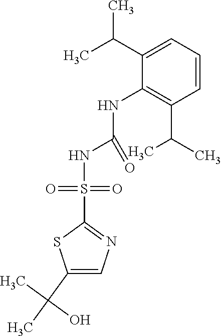 [SMILES: CC(C)c1cccc(C(C)C)c1NC(=O)NS(=O)(=O)c1ncc(C(C)(C)O)s1] |
| 228 | 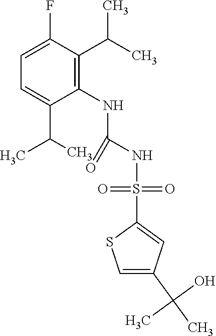 [SMILES: CC(C)c1ccc(F)c(C(C)C)c1NC(=O)NS(=O)(=O)c1cc(C(C)(C)O)cs1] |
| 229 | 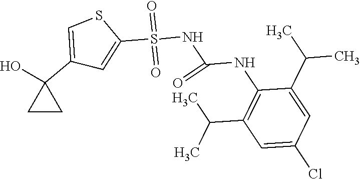 [SMILES: CC(C)c1cc(Cl)cc(C(C)C)c1NC(=O)NS(=O)(=O)c1cc(C2(O)CC2)cs1] |
| 230 | 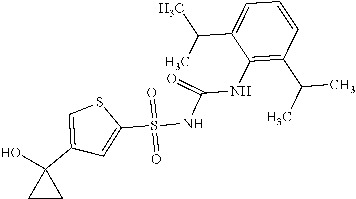 [SMILES: CC(C)c1cccc(C(C)C)c1NC(=O)NS(=O)(=O)c1cc(C2(O)CC2)cs1] |
| 232 | 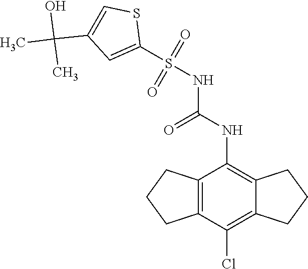 [SMILES: CC(C)(O)c1csc(S(=O)(=O)NC(=O)Nc2c3c(c(Cl)c4c2CCC4)CCC3)c1] |
| 233 | 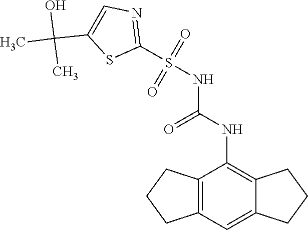 [SMILES: CC(C)(O)c1cnc(S(=O)(=O)NC(=O)Nc2c3c(cc4c2CCC4)CCC3)s1] |
| 235 | 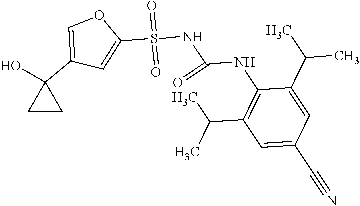 [SMILES: CC(C)c1cc(C#N)cc(C(C)C)c1NC(=O)NS(=O)(=O)c1cc(C2(O)CC2)co1] |
| 236 | 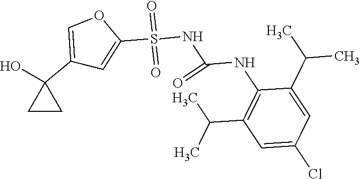 [SMILES: CC(C)c1cc(Cl)cc(C(C)C)c1NC(=O)NS(=O)(=O)c1cc(C2(O)CC2)co1] |
| 239 | 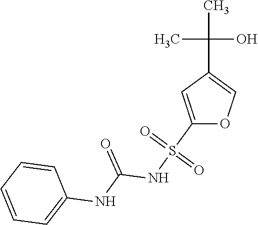 [SMILES: CC(C)(O)c1coc(S(=O)(=O)NC(=O)Nc2ccccc2)c1] |
| 240 | 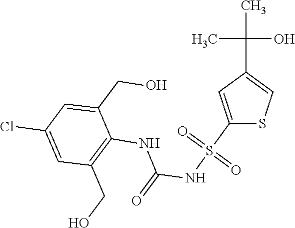 [SMILES: CC(C)(O)c1csc(S(=O)(=O)NC(=O)Nc2c(CO)cc(Cl)cc2CO)c1] |
| 241 | 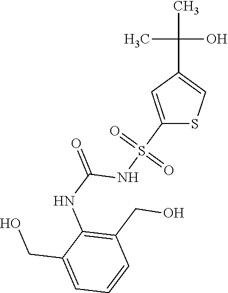 [SMILES: CC(C)(O)c1csc(S(=O)(=O)NC(=O)Nc2c(CO)cccc2CO)c1] |
| 242 | 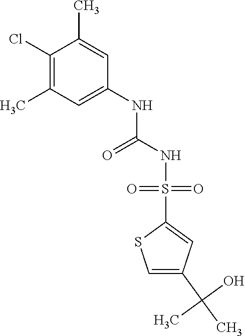 [SMILES: Cc1cc(NC(=O)NS(=O)(=O)c2cc(C(C)(C)O)cs2)cc(C)c1Cl] |
| 243 | 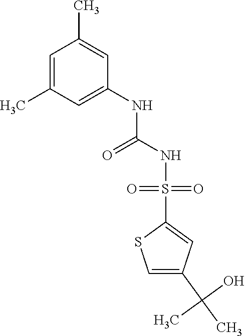 [SMILES: Cc1cc(C)cc(NC(=O)NS(=O)(=O)c2cc(C(C)(C)O)cs2)c1] |
| 244 | 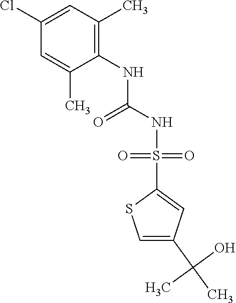 [SMILES: Cc1cc(Cl)cc(C)c1NC(=O)NS(=O)(=O)c1cc(C(C)(C)O)cs1] |
| 247 | 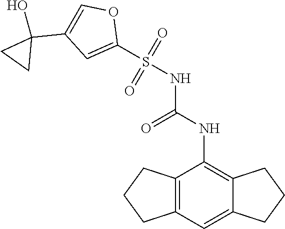 [SMILES: O=C(Nc1c2c(cc3c1CCC3)CCC2)NS(=O)(=O)c1cc(C2(O)CC2)co1] |
| 250 | 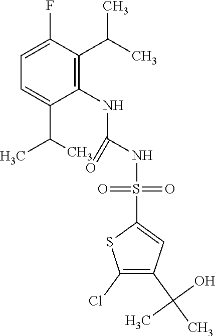 [SMILES: CC(C)c1ccc(F)c(C(C)C)c1NC(=O)NS(=O)(=O)c1cc(C(C)(C)O)c(Cl)s1] |
| 251 | 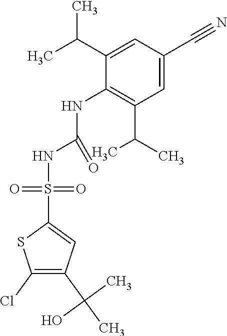 [SMILES: CC(C)c1cc(C#N)cc(C(C)C)c1NC(=O)NS(=O)(=O)c1cc(C(C)(C)O)c(Cl)s1] |
| 252 | 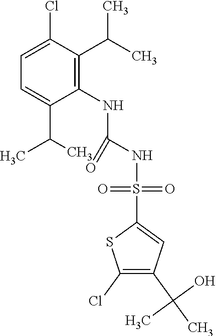 [SMILES: CC(C)c1ccc(Cl)c(C(C)C)c1NC(=O)NS(=O)(=O)c1cc(C(C)(C)O)c(Cl)s1] |
| 253 | 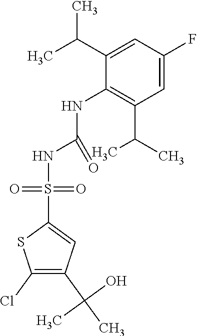 [SMILES: CC(C)c1cc(F)cc(C(C)C)c1NC(=O)NS(=O)(=O)c1cc(C(C)(C)O)c(Cl)s1] |
| 254 | 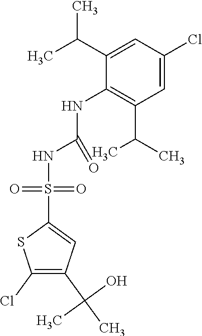 [SMILES: CC(C)c1cc(Cl)cc(C(C)C)c1NC(=O)NS(=O)(=O)c1cc(C(C)(C)O)c(Cl)s1] |
| 255 | 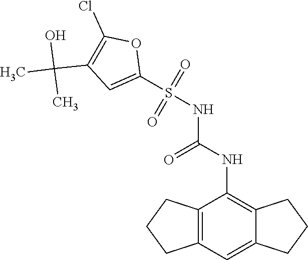 [SMILES: CC(C)(O)c1cc(S(=O)(=O)NC(=O)Nc2c3c(cc4c2CCC4)CCC3)oc1Cl] |
| 257 | 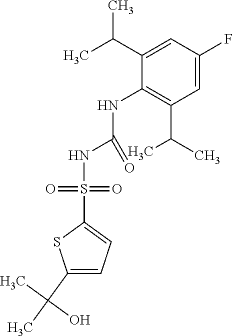 [SMILES: CC(C)c1cc(F)cc(C(C)C)c1NC(=O)NS(=O)(=O)c1ccc(C(C)(C)O)s1] |
| 258 | 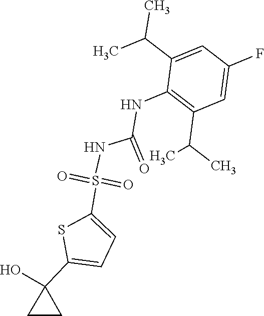 [SMILES: CC(C)c1cc(F)cc(C(C)C)c1NC(=O)NS(=O)(=O)c1ccc(C2(O)CC2)s1] |
| 259 | 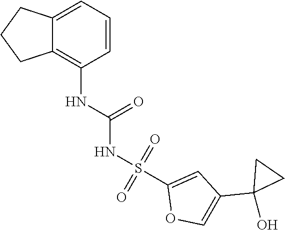 [SMILES: O=C(Nc1cccc2c1CCC2)NS(=O)(=O)c1cc(C2(O)CC2)co1] |
| 260 | 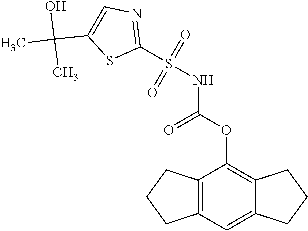 [SMILES: CC(C)(O)c1cnc(S(=O)(=O)NC(=O)Oc2c3c(cc4c2CCC4)CCC3)s1] |
| 261 | 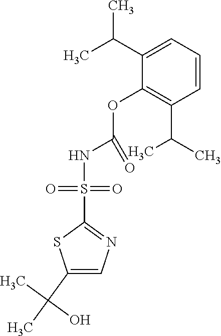 [SMILES: CC(C)c1cccc(C(C)C)c1OC(=O)NS(=O)(=O)c1ncc(C(C)(C)O)s1] |
| 262 | 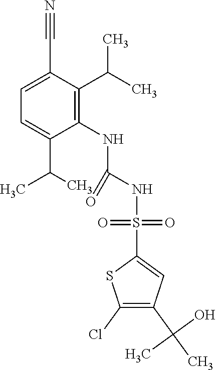 [SMILES: CC(C)c1ccc(C#N)c(C(C)C)c1NC(=O)NS(=O)(=O)c1cc(C(C)(C)O)c(Cl)s1] |
| 263 | 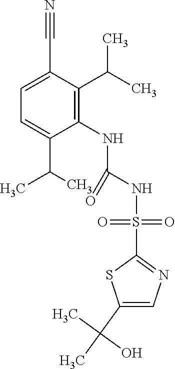 [SMILES: CC(C)c1ccc(C#N)c(C(C)C)c1NC(=O)NS(=O)(=O)c1ncc(C(C)(C)O)s1] |
| 264 | 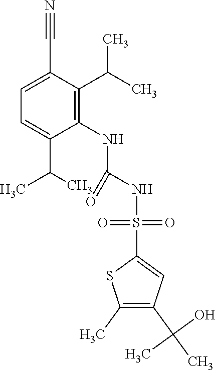 [SMILES: Cc1sc(S(=O)(=O)NC(=O)Nc2c(C(C)C)ccc(C#N)c2C(C)C)cc1C(C)(C)O] |
| 265 | 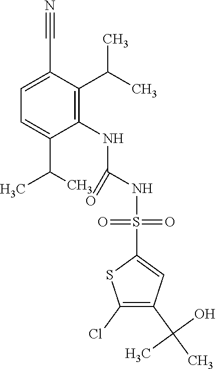 [SMILES: CC(C)c1ccc(C#N)c(C(C)C)c1NC(=O)NS(=O)(=O)c1cc(C(C)(C)O)c(Cl)s1] |
| 266 | 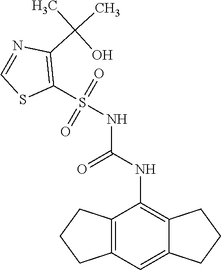 [SMILES: CC(C)(O)c1ncsc1S(=O)(=O)NC(=O)Nc1c2c(cc3c1CCC3)CCC2] |
| 267 | 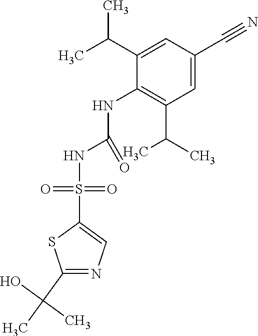 [SMILES: CC(C)c1cc(C#N)cc(C(C)C)c1NC(=O)NS(=O)(=O)c1cnc(C(C)(C)O)s1] |
| 268 | 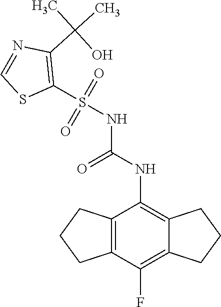 [SMILES: CC(C)(O)c1ncsc1S(=O)(=O)NC(=O)Nc1c2c(c(F)c3c1CCC3)CCC2] |
| 269 | 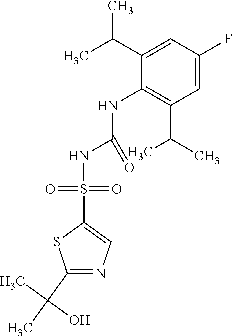 [SMILES: CC(C)c1cc(F)cc(C(C)C)c1NC(=O)NS(=O)(=O)c1cnc(C(C)(C)O)s1] |
| 270 | 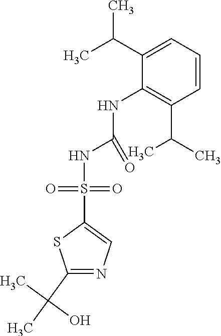 [SMILES: CC(C)c1cccc(C(C)C)c1NC(=O)NS(=O)(=O)c1cnc(C(C)(C)O)s1] |
| 271 | 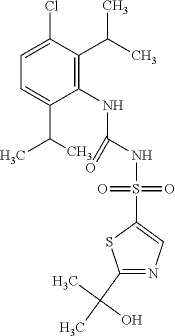 [SMILES: CC(C)c1ccc(Cl)c(C(C)C)c1NC(=O)NS(=O)(=O)c1cnc(C(C)(C)O)s1] |
| 272 | 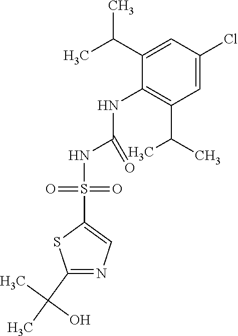 [SMILES: CC(C)c1cc(Cl)cc(C(C)C)c1NC(=O)NS(=O)(=O)c1cnc(C(C)(C)O)s1] |
| 273 | 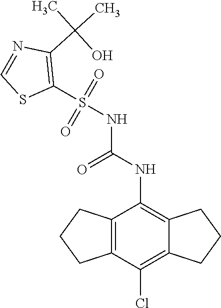 [SMILES: CC(C)(O)c1ncsc1S(=O)(=O)NC(=O)Nc1c2c(c(Cl)c3c1CCC3)CCC2] |
| 274 | 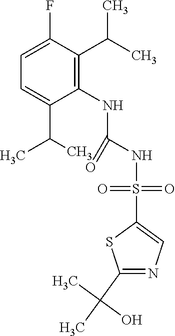 [SMILES: CC(C)c1ccc(F)c(C(C)C)c1NC(=O)NS(=O)(=O)c1cnc(C(C)(C)O)s1] |

Compounds having Formula I and II, as well as methods of making and using the same, are further described in WO2017184624A1 (PCT/US2017/028167), filed on Apr. 18, 2017; U.S. Provisional 62/324,081, filed on Apr. 18, 2016; and U.S. Provisional 62/324,071, filed on Apr. 18, 2016, each of which is incorporated herein by reference in its entirety.

In one aspect, an NLRP3 antagonist is a compound of Formula III

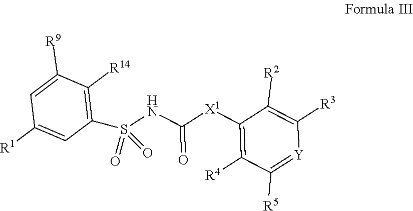

or a pharmaceutically acceptable salt thereof, wherein:

X¹ is NH or O;

or when X¹ is NH, X¹ and R² taken together with the atoms connecting them form a four-to-seven-membered heterocyclic ring optionally substituted with one or more R¹⁶;

or when X¹ is NH, X¹ and R⁴ taken together with the atoms connecting them form a four-to-seven-membered heterocyclic ring optionally substituted with one or more R¹⁶;

Y is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R³ is hydrogen, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

provided that at least one of R², R³, R⁴ and R⁵ is not hydrogen, and that R² and R⁴ are not both hydroxymethyl;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

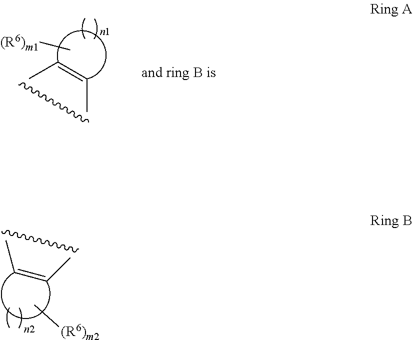

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from H, CN, Cl, or F;

R¹⁴ is selected from H, CN, Cl, or F;

R⁹ is selected from C₁-C₆ alkyl, C(R¹⁰)₂OH, C(R¹⁰)₂NR¹¹R¹², C₃-C₆ cycloalkyl and C₃-C₆ heterocycloalkyl;

wherein, when R⁹ is C₁-C₆ alkyl, C₃-C₆ cycloalkyl or C₃-C₆ heterocycloalkyl, R⁹ is optionally substituted with one or more substituents each independently selected from ═NR¹³, COOC₁-C₆ alkyl, and CONR¹¹R¹²;

each R¹⁰ is the same and is H or C₁-C₆ alkyl;

or two R¹⁰ taken together with the carbon connecting them form a three-to-eight-membered heterocyclic ring containing 1 or 2 heteroatoms independently selected from O, N, and S, or a three-membered, six-membered, seven-membered, or eight-membered carbocyclic ring, wherein the heterocyclic ring or carbocyclic ring is optionally substituted with one or more substituents each independently selected from H, C₁-C₆ alkyl, C₁-C₆ alkoxy, NR¹¹R¹², oxo, and ═NR¹³;

R¹³ is C₁-C₆ alkyl;

each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;
each of R¹⁷ and R¹⁸ at each occurrence is independently selected from hydrogen and C₁-C₆ alkyl;
each R¹⁶ is the same or different and is selected from H, C₁-C₆ alkyl, C₁-C₆ alkoxy, NR¹¹R¹², oxo, and ═NR¹³;

provided that if R² and R⁴ are each isopropyl; X¹ is NH; each R¹⁰ is C₁-C₆ alkyl; R¹⁴ is H; and R¹ is H, then R¹ is not F or Cl.

In another aspect, an NLRP3 antagonist is a compound of Formula III

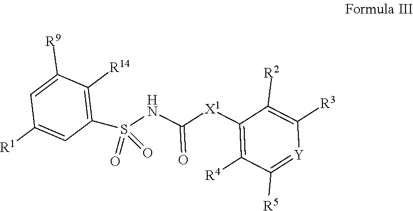

or a pharmaceutically acceptable salt thereof, wherein:

X¹ is NH or O;

or when X¹ is NH, X¹ and R² taken together with the atoms connecting them form a four-to-seven-membered heterocyclic ring optionally substituted with one or more R¹⁶; or when X¹ is NH, X¹ and R⁴ taken together with the atoms connecting them form a four-to-seven-membered heterocyclic ring optionally substituted with one or more R¹⁶;

Y is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R³ is hydrogen, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

provided that at least one of R², R³, R⁴ and R⁵ is not hydrogen, and that R² and R⁴ are not both hydroxymethyl;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

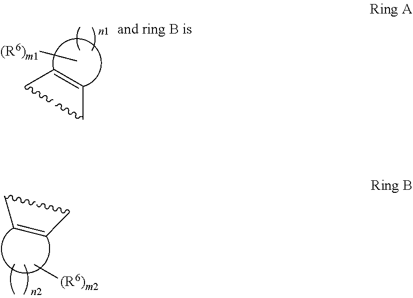

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from H, CN, Cl, or F;

R¹⁴ is selected from H, CN, Cl, or F;

R⁹ is selected from C₁-C₆ alkyl, C(R¹⁰)₂OH, C(R¹⁰)₂NR¹¹R¹², C₃-C₆ cycloalkyl and C₃-C₆ heterocycloalkyl;

wherein, when R⁹ is C₁-C₆ alkyl, C₃-C₆ cycloalkyl or C₃-C₆ heterocycloalkyl, R⁹ is optionally substituted with one or more substituents each independently selected from ═NR¹³, COOC₁-C₆ alkyl, and CONR¹¹R¹²;

each R¹⁰ is the same and is H or C₁-C₆ alkyl;

R¹³ is C₁-C₆ alkyl;

each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl;

each R¹⁶ is the same or different and is selected from H, C₁-C₆ alkyl, C₁-C₆ alkoxy, NR¹¹R¹², oxo, and ═NR¹³;

provided that if R² and R⁴ are each isopropyl; X¹ is NH; each R¹⁰ is C₁-C₆ alkyl; R¹⁴ is H; and R¹ is H, then R¹ is not F or Cl.

In another aspect, an NLRP3 antagonist is a compound of Formula III

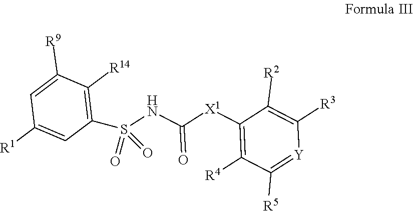

or a pharmaceutically acceptable salt thereof, wherein:

X¹ is NH or O;

or when X¹ is NH, X¹ and R² taken together with the atoms connecting them form a four-to-seven-membered heterocyclic ring optionally substituted with one or more R¹⁶; or when X¹ is NH, X¹ and R⁴ taken together with the atoms connecting them form a four-to-seven-membered heterocyclic ring optionally substituted with one or more R¹⁶;

Y is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R³ is hydrogen, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

provided that at least one of R², R³, R⁴ and R⁵ is not hydrogen, and that R² and R⁴ are not both hydroxymethyl;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

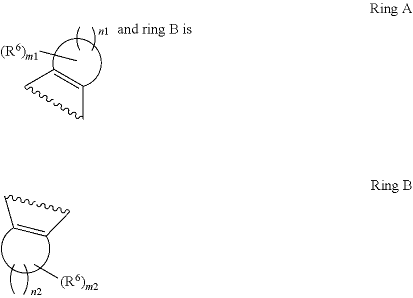

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from H, CN, Cl, or F;

R¹⁴ is selected from H, CN, Cl, or F;

R⁹ is selected from C₁-C₆ alkyl)C(R¹⁰)₂OH, C(R¹⁰)₂NR¹¹R¹², C₃-C₆ cycloalkyl and C₃-C₆ heterocycloalkyl;

wherein, when R⁹ is C₁-C₆ alkyl, C₃-C₆ cycloalkyl or C₃-C₆ heterocycloalkyl, R⁹ is optionally substituted with one or more substituents each independently selected from ═NR¹³, COOC₁-C₆ alkyl, and CONR¹¹R¹²;

each R¹⁰ is the same and is H or C₁-C₆ alkyl;

R¹³ is C₁-C₆ alkyl;

each of R¹¹ and R¹² at each occurrence is independently selected from hydrogen,

C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl;

each R¹⁶ is the same or different and is selected from H, C₁-C₆ alkyl, C₁-C₆ alkoxy, NR¹¹R¹², oxo, and ═NR¹³;
provided that if R² and R⁴ are each isopropyl; X¹ is NH; each R¹⁰ is C₁-C₆ alkyl; and R¹ is H, then R⁸ is not F or Cl.

In another aspect, an NLRP3 antagonist is a compound of Formula III

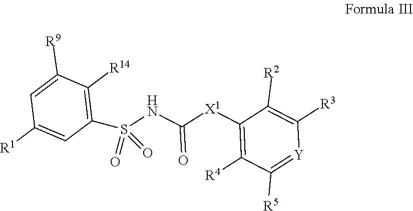

or a pharmaceutically acceptable salt thereof, wherein:

X¹ is NH or O;

or when X¹ is NH, X¹ and R² taken together with the atoms connecting them form a four-to-seven-membered heterocyclic ring optionally substituted with one or more R¹⁶;

Y is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R³ is hydrogen, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

provided that at least one of R², R³, R⁴ and R⁵ is not hydrogen, and that R² and R⁴ are not both hydroxymethyl;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

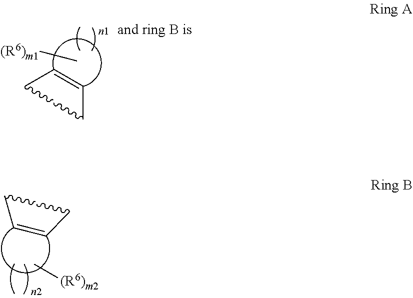

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from H, CN, Cl, or F;

R¹⁴ is selected from H, CN, Cl, or F;

R⁹ is selected from C₁-C₆ alkyl, C(R¹⁰)₂OH, C(R¹⁰)₂NR¹¹R¹², C₃-C₆ cycloalkyl and C₃-C₆ heterocycloalkyl;

wherein, when R⁹ is C₁-C₆ alkyl, C₃-C₆ cycloalkyl or C₃-C₆ heterocycloalkyl, R⁹ is optionally substituted with one or more substituents each independently selected from ═NR¹³, COOC₁-C₆ alkyl, and CONR¹¹R¹²;

each R¹⁰ is the same and is H or C₁-C₆ alkyl;

R¹³ is C₁-C₆ alkyl;
each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl;

each R¹⁶ is the same or different and is selected from H, C₁-C₆ alkyl, C₁-C₆ alkoxy, NR¹¹R¹², oxo, and ═NR¹³;

provided that if R² and R⁴ are each isopropyl; X′ is NH; each R¹⁰ is C₁-C₆ alkyl; and R¹ is H, then R⁸ is not F or Cl.

In another aspect, an NLRP3 antagonist is a compound of Formula III

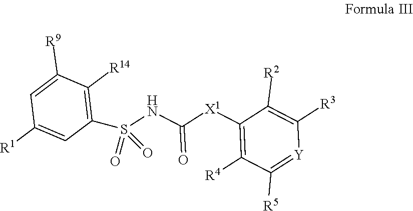

or a pharmaceutically acceptable salt thereof, wherein:

X¹ is NH or O;

Y is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R³ is hydrogen, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

provided that at least one of R², R³, R⁴ and R⁵ is not hydrogen, and that R² and R⁴ are not both hydroxymethyl;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

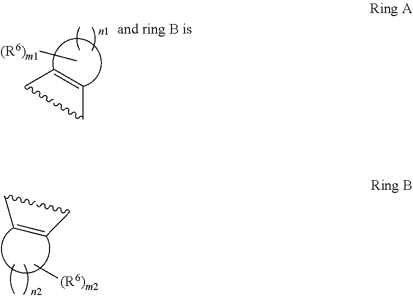

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from H, CN, Cl, or F;

R¹⁴ is selected from H, CN, Cl, or F;

R⁹ is selected from C₁-C₆ alkyl, C(R¹⁰)₂OH, C(R¹⁰)₂NR¹¹R¹², C₃-C₆ cycloalkyl and C₃-C₆ heterocycloalkyl;

wherein, when R⁹ is C₁-C₆ alkyl, C₃-C₆ cycloalkyl or C₃-C₆ heterocycloalkyl, R⁹ is optionally substituted with one or more substituents each independently selected from ═NR¹³, COOC₁-C₆ alkyl, and CONR¹¹R¹²;

each R¹⁰ is the same and is H or C₁-C₆ alkyl;

each of R¹¹, R¹² and R¹³ at each occurrence is independently selected from hydrogen and C₁-C₆ alkyl;

provided that if R² and R⁴ are each isopropyl; X¹ is NH; each R¹⁰ is C₁-C₆ alkyl; R¹⁴ is H; and R¹ is H, then R¹ is not F or Cl.

In another aspect, an NLRP3 antagonist is a compound of Formula III

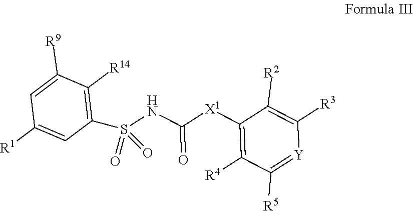

or a pharmaceutically acceptable salt thereof, wherein:

X¹ is NH or O;

Y is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R³ is hydrogen, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

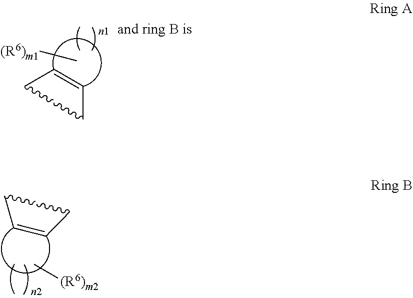

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from H, CN, Cl, or F;

R¹⁴ is selected from H, CN, Cl, or F;

R⁹ is selected from C₁-C₆ alkyl, C(R¹⁰)₂OH, C(R¹⁰)₂NR¹¹R¹², C₃-C₆ cycloalkyl and C₃-C₆ heterocycloalkyl;

wherein, when R⁹ is C₁-C₆ alkyl, C₃-C₆ cycloalkyl or C₃-C₆ heterocycloalkyl, R⁹ is optionally substituted with one or more substituents each independently selected from ═NR¹³, COOC₁-C₆ alkyl, and CONR¹¹R¹²;

each R¹⁰ is the same and is H or C₁-C₆ alkyl;

each of R¹¹, R¹² and R¹³ at each occurrence is independently selected from hydrogen and C₁-C₆ alkyl;

provided that if R² and R⁴ are each isopropyl; X¹ is NH; each R¹⁰ is C₁-C₆ alkyl; and R¹ is H, then R⁸ is not F or Cl.

In some embodiments of the compound of Formula III, X¹ is NH.

In some embodiments of the compound of Formula III, X¹ is O.

In some embodiments of the compound of Formula III, X¹ and R² taken together with the atoms connecting them form a four-to-seven-membered heterocyclic ring optionally substituted with one or more R¹⁶.

In certain of the foregoing embodiments of Formula III, X¹ and R² taken together with the atoms connecting them form a four-to-seven-membered heterocyclic ring optionally substituted with one or more C₁-C₆ alkyl.

In some embodiments of the compound of Formula III, X¹ and R⁴ taken together with the atoms connecting them form a four-to-seven-membered heterocyclic ring optionally substituted with one or more oxo.

In certain embodiments of the compound of Formula III, X¹ and R⁴ taken together with the atoms connecting them form a four-to-seven-membered heterocyclic ring optionally substituted with one or more methyl.

In certain embodiments of the compound of Formula III, X¹ and R⁴ taken together with the atoms connecting them form a four-to-seven-membered heterocyclic ring optionally substituted with one or more C₁-C₆ alkoxy.

In some embodiments of the compound of Formula III, Y is CR⁸.

In some embodiments of the compound of Formula III, Y is N.

In some embodiments of the compound of Formula III, R² is hydrogen.

In some embodiments of the compound of Formula III, R² is C₁-C₆ alkyl optionally substituted with hydroxy. For example, R² can be isopropyl; or R² can be methyl.

In some embodiments of the compound of Formula III, R³ is hydrogen.

In some embodiments of the compound of Formula III, R³ is C₁-C₆ alkyl optionally substituted with hydroxy. For example, R³ can be isopropyl; or R³ can be methyl.

In some embodiments of the compound of Formula III, R⁴ is hydrogen.

In some embodiments of the compound of Formula III, R⁴ is C₁-C₆ alkyl optionally substituted with hydroxy. For example, R⁴ can be isopropyl; or R⁴ can be methyl.

In some embodiments of the compound of Formula III, R⁵ is hydrogen.

In some embodiments of the compound of Formula III, R⁵ is C₁-C₆ alkyl optionally substituted with hydroxy. For example, R⁵ can be isopropyl; or R⁵ can be methyl.

In some embodiments of the compound of Formula III, R² and R³ taken together with the carbons connecting them form ring A.

In certain of the foregoing embodiments of Formula III, ring A is

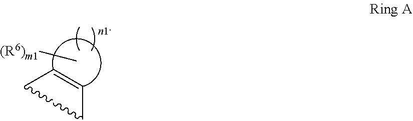

In some embodiments of the compound of Formula III, R² and R³ taken together with the carbons connecting them form ring B.

In certain of the foregoing embodiments of Formula III, ring B is

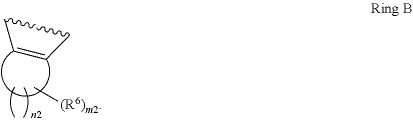

In some embodiments of the compound of Formula III (when one pair of R² and R³ taken together with the carbons connecting them form ring A; and another pair R² and R³ taken together with the carbons connecting them form ring B), ring A is the same as ring B.

In some embodiments of the compound of Formula III, n1 is 3.

In some embodiments of the compound of Formula III, n1 is 4.

In some embodiments of the compound of Formula III, n2 is 3.

In some embodiments of the compound of Formula III, n2 is 4.

In some embodiments of the compound of Formula III, R⁶ is H.

In some embodiments of the compound of Formula III, R⁸ is H; R⁸ is CN; R⁸ is C₁; R⁸ is F; R⁸ is C₁-C₆ alkyl; or R⁸ is C₁-C₆ haloalkyl (e.g., R⁸ can be CF₃).

In some embodiments of the compound of Formula III, R¹ is H; R¹ is CN; R¹ is C₁; or R¹ is F.

In some embodiments of the compound of Formula III, 104^{is} H; R¹⁴ is CN; R¹⁴ is Cl; or R¹⁴ is F.

In some embodiments of the compound of Formula III, R⁹ is C(R¹⁰)₂OH.

In some embodiments of the compound of Formula III, R⁹ is C(R¹⁰)₂NR¹¹R¹².

In some embodiments of the compound of Formula III, R⁹ is C₁-C₆ alkyl.

In some embodiments of the compound of Formula III, R⁹ is C₃-C₆ cycloalkyl.

In some embodiments of the compound of Formula III, each R¹⁰ is H.

In some embodiments of the compound of Formula III, each R¹⁰ is C₁-C₆ alkyl. For example, each R¹⁰ can be methyl.

In some embodiments of the compound of Formula III, two R¹⁰ taken together with the carbon connecting them form a three-to-eight-membered heterocyclic ring containing 1 or 2 heteroatoms independently selected from O, N, and S.

In some embodiments of the compound of Formula III, two R¹⁰ taken together with the carbon connecting them form a three-membered, six-membered, seven-membered, or eight-membered carbocyclic ring.

In some embodiments of the compound of Formula III, the heterocyclic or carbocyclic ring formed by the two R¹⁰ is optionally substituted with one or more substituents each independently selected from H, C₁-C₆ alkyl, C₁-C₆ alkoxy, NR¹¹R¹², and ═NR¹³.

In some embodiments of the compound of Formula III, each R¹¹ is hydrogen.

In some embodiments of the compound of Formula III, each R¹¹ is C₁-C₆ alkyl.

In some embodiments of the compound of Formula III, each R¹² is hydrogen.

In some embodiments of the compound of Formula III, each R¹² is C₁-C₆ alkyl.

In some embodiments of the compound of Formula III, each R¹⁶ is hydrogen.

In some embodiments of the compound of Formula III, if R² and R⁴ are each isopropyl; X¹ is NH; each R¹⁰ is C₁-C₆ alkyl; R¹⁴ is H; and R¹ is H, then R⁸ is not F or Cl.

In some embodiments of the compound of Formula III, if R² and R⁴ are each isopropyl; X¹ is NH; each R¹⁰ is C₁-C₆ alkyl; and R¹ is H, then R⁸ is not F or Cl.

In some embodiments of the compound of Formula III, the compound is a compound of Formula III(A):

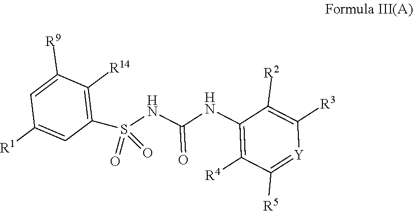

or a pharmaceutically acceptable salt thereof, wherein

Y is N or CR⁸;

R⁸ is selected from H, CN, Cl, F, CO₂C₁-C₆ alkyl and CONH₂;

R² is hydrogen, C₁-C₆ alkoxy, halo, C₁-C₆ haloalkyl, or C₁-C₆ alkyl optionally substituted with hydroxy;

R³ is hydrogen, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

provided that at least one of R², R³, R⁴ and R⁵ is not hydrogen, and that R² and R⁴ are not both hydroxymethyl;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

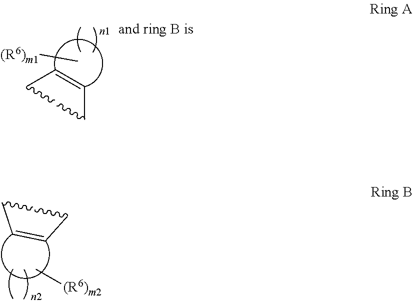

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, C₁-C₆ alkyl, C₁-C₆ alkoxy, oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from H, CN, Cl, or F;

R¹⁴ is selected from H, CN, Cl, or F;

R⁹ is selected from C₁-C₆ alkyl, C(R¹)₂OH, C(R¹⁰)₂NR¹¹R¹², and C₃-C₆ cycloalkyl;

each R¹⁰ is the same and is H or C₁-C₆ alkyl;

each of R¹¹, R¹² and R¹³ at each occurrence is independently selected from hydrogen and C₁-C₆ alkyl;

provided that if R² and R⁴ are each isopropyl; X¹ is NH; each R¹⁰ is C₁-C₆ alkyl; R¹⁴ is H; and R¹ is H, then R¹ is not F or Cl.

In some embodiments of the compound of Formula III, the compound is a compound of Formula III(A):

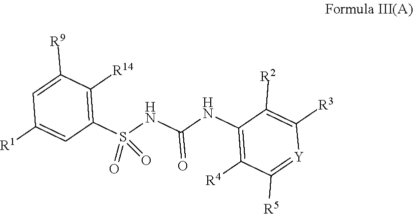

or a pharmaceutically acceptable salt thereof, wherein

Y is N or CR⁸;

R⁸ is selected from H, CN, Cl, F, CO₂C₁-C₆ alkyl and CONH₂;

R² is hydrogen, C₁-C₆ alkoxy, halo, C₁-C₆ haloalkyl, or C₁-C₆ alkyl optionally substituted with hydroxy;

R³ is hydrogen, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, C₁-C₆ alkoxy, halo, C₁-C₆ haloalkyl, or C₁-C₆ alkyl optionally substituted with hydroxy;

provided that at least one of R², R³, R⁴ and R⁵ is not hydrogen, and that R² and R⁴ are not both hydroxymethyl;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

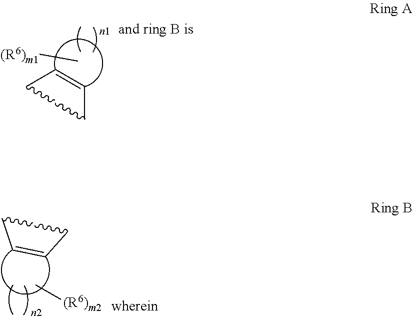

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from H, CN, Cl, or F;

R¹⁴ is selected from H, CN, Cl, or F;

R⁹ is selected from C₁-C₆ alkyl, C(R¹⁰)₂OH, C(R¹⁰)₂NR¹¹R¹², and C₃-C₆ cycloalkyl; each R¹⁰ is the same and is H or C₁-C₆ alkyl;

each of R¹¹, R¹² and R¹³ at each occurrence is independently selected from hydrogen and C₁-C₆ alkyl;

provided that if R² and R⁴ are each isopropyl; X¹ is NH; each R¹⁰ is C₁-C₆ alkyl; and R¹ is H, then R⁸ is not F or Cl.

In some embodiments of the compound of Formula III, the compound is a compound of Formula III(A)-i:

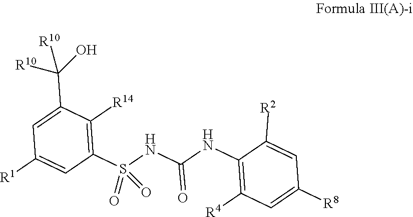

or a pharmaceutically acceptable salt thereof.

In some embodiments of the compound of Formula III, the compound is a compound of Formula III(A)-ii:

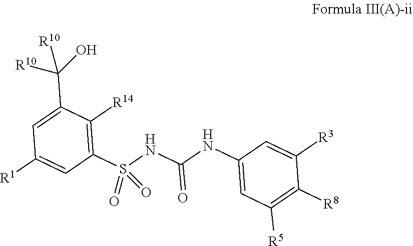

or a pharmaceutically acceptable salt thereof.

In some embodiments of the compound of Formula III, the compound is a compound of Formula III(B):

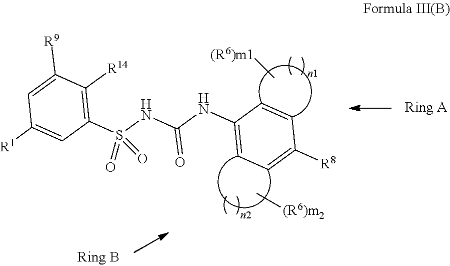

wherein

R⁸ is selected from H, CN, Cl, F, CO₂C₁-C₆ alkyl and CONH₂;

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, C₁-C₆ alkyl, C₁-C₆ alkoxy, oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from H, CN, Cl, or F;

R¹⁴ is selected from H, CN, Cl, or F;

R⁹ is selected from C₁-C₆ alkyl, C(R¹⁰)₂OH)C(R¹⁰)₂NR¹¹R¹², and cycloalkyl;

each R¹⁰ is the same and is H or C₁-C₆ alkyl;

and each of R¹¹, R¹² and R¹³ at each occurrence is independently selected from hydrogen and C₁-C₆ alkyl.

In some embodiments of the compound of Formula III, the compound is a compound of Formula III(B)-i:

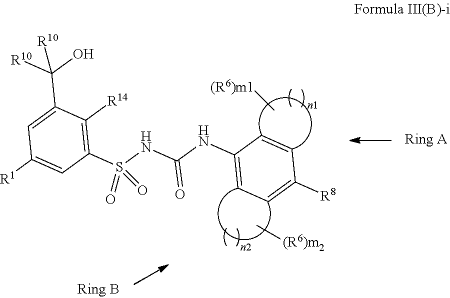

or a pharmaceutically acceptable salt thereof.

In some embodiments of the compound of Formula III, the compound is a compound of Formula III(B)-ii:

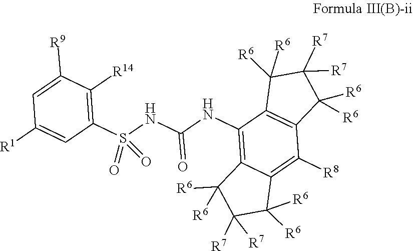

or a pharmaceutically acceptable salt thereof.

In some embodiments of the compound of Formula III, the compound is a compound of Formula III(B)-iii:

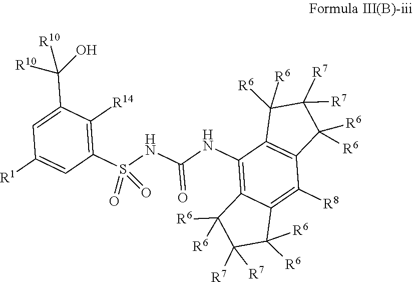

or a pharmaceutically acceptable salt thereof.

In another aspect, an NLRP3 antagonist is a compound of Formula IV

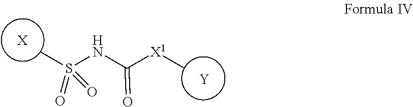

wherein the compound of Formula II is selected from

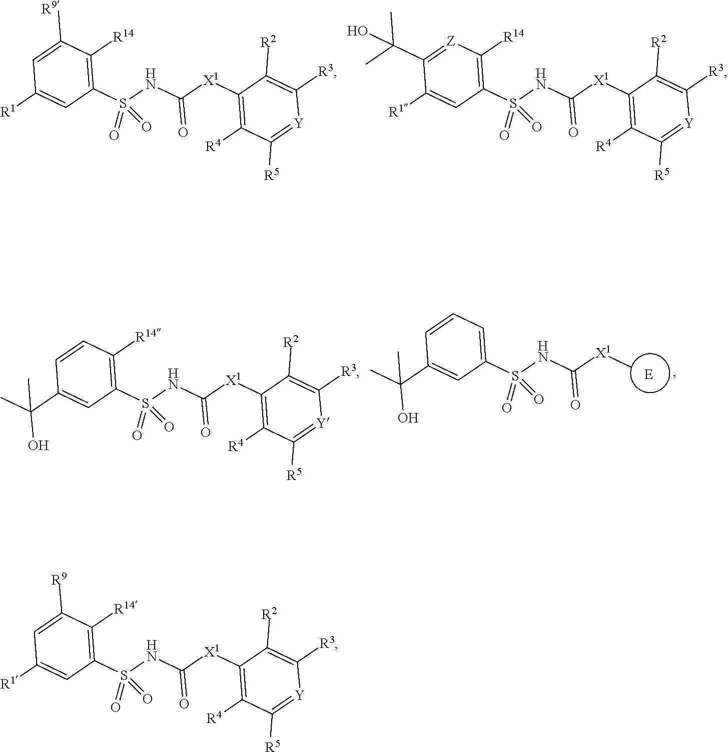

or a pharmaceutically acceptable salt thereof, wherein:

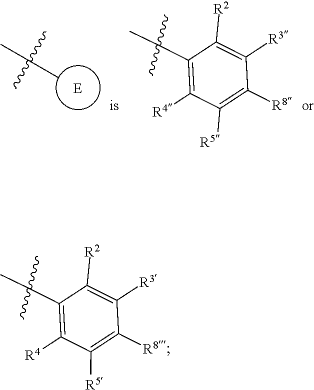

X¹ is NH or O;

or when X¹ is NH, X¹ and R² taken together with the atoms connecting them form a four-to-seven-membered heterocyclic ring optionally substituted with one or more R¹⁶;

or when X¹ is NH, X¹ and R⁴ taken together with the atoms connecting them form a four-to-seven-membered heterocyclic ring optionally substituted with one or more R¹⁶;

Y is N or CR⁸;

Y′ is N or CR^{8′};

Z is N or CH;

R⁸ is selected from H, CN, Cl, F, CO₂C₁-C₆ alkyl, CO₂C₃-C₈ cycloalkyl, CONR¹¹R¹², C₁-C₆ alkyl, C₁-C₆ haloalkoxy, and C₁-C₆ haloalkyl;

R^{8′} is selected from H, CN, F, CO₂C₁-C₆ alkyl, CO₂C₃-C₈ cycloalkyl, CONR¹¹R¹², C₁-C₆ alkyl, and C₁-C₆ haloalkyl;

R^{8″} is selected from CN, Cl, F, CO₂C₁-C₆ alkyl, CO₂C₃-C₈ cycloalkyl, CONR¹¹R¹², C₁-C₆ alkyl, and C₁-C₆ haloalkyl;

R^{8′″} is selected from H, CN, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, or C₁-C₆ alkyl optionally substituted with hydroxy;

R³ is hydrogen, CN, C₁-C₆ alkoxy, halo, C₁-C₆ haloalkyl, or C₁-C₆ alkyl optionally substituted with hydroxy;

R^{3′} is hydrogen, CN, C₁-C₆ alkoxy, halo, C₁-C₆ haloalkyl, or C₁-C₆ alkyl optionally substituted with hydroxy;

R⁴ is hydrogen, C₁-C₆ alkoxy, halo, C₁-C₆ haloalkyl, or C₁-C₆ alkyl optionally substituted with hydroxy;

R⁵ is hydrogen, CN, C₁-C₆ alkoxy, halo, C₁-C₆ haloalkyl, or C₁-C₆ alkyl optionally substituted with hydroxy;

R^{5′} is hydrogen, CN, C₁-C₆ alkoxy, halo, C₁-C₆ haloalkyl, or C₁-C₆ alkyl optionally substituted with hydroxy;

provided that at least one of R², R³, R⁴ and R⁵ is not hydrogen, and that R² and R⁴ are not both hydroxymethyl;

or R² and R³, if both present, taken together with the carbons connecting them form a four-membered to seven-membered ring A,

or R⁴ and R⁵, if both present, taken together with the carbons connecting them form a four-membered to seven-membered ring B,

or R² and R³, if both present, taken together with the carbons connecting them form a four-membered to seven-membered ring A and R⁴ and R⁵, if both present, taken together with the carbons connecting them form a four-membered to seven-membered ring B,

R^{2″} and R^{3″}, if both present, taken together with the carbons connecting them form a four-membered to seven-membered ring A and R^{4″} and R^{5″}, if both present, taken together with the carbons connecting them form a four-membered to seven-membered ring B,

wherein ring A is

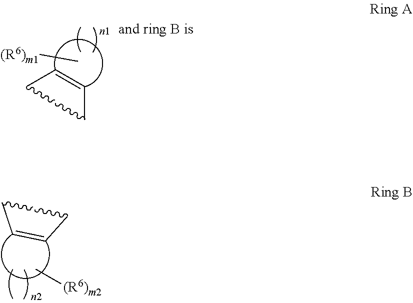

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

R¹ is selected from CN, Cl, C(R¹⁰)₂OH, or F;

R^{1′} is selected from C(O)C₁-C₆ alkyl or CO₂C₁-C₆ alkyl;

R^{1″} is selected from H or C(R¹⁰)₂OH;

R¹⁴ is selected from H, CN, Cl, or F;

R^{14′} is selected from CN or F;

R^{14″} is selected from CN, Cl, or F;

R⁹ is selected from H, C₁-C₆ alkyl, C(R¹⁰)₂OH, C(R¹⁰)₂NR¹¹R¹², C₃-C₆ cycloalkyl, pyridyl, and morpholinyl;

wherein, when R⁹ is C₁-C₆ alkyl, C₃-C₆ cycloalkyl or C₃-C₆ heterocycloalkyl, R⁹ is optionally substituted with one or more substituents each independently selected from ═NR¹³, COOC₁-C₆ alkyl, and CONR¹¹R¹²;

R^{9′} is selected from C₁-C₆ alkyl, C(R¹⁰)₂OH, C(R¹⁰)₂NR¹¹R¹², C₃-C₆ cycloalkyl, phenyl, pyridyl, and morpholinyl; each R¹⁰ is the same and is H or C₁-C₆ alkyl;

or two R¹⁰ taken together with the carbon connecting them form a three-to-eight-membered heterocyclic ring containing 1 or 2 heteroatoms independently selected from O, N, and S, or a three-membered, six-membered, seven-membered, or eight-membered carbocyclic ring, wherein the heterocyclic ring or carbocyclic ring is optionally substituted with one or more substituents each independently selected from H, C₁-C₆ alkyl, C₁-C₆ alkoxy, NR¹¹R¹², oxo, and ═NR¹³;

R¹³ is C₁-C₆ alkyl;

each of R¹¹ and R¹² at each occurrence is independently selected from hydrogen,

C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl;

each R¹⁶ is the same or different and is selected from H, C₁-C₆ alkyl, C₁-C₆ alkoxy, NR¹¹R¹² oxo, and ═NR¹³.

In another aspect, an NLRP3 antagonist is a compound of Formula IV

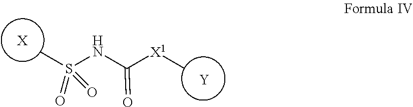

wherein the compound of Formula II is selected from

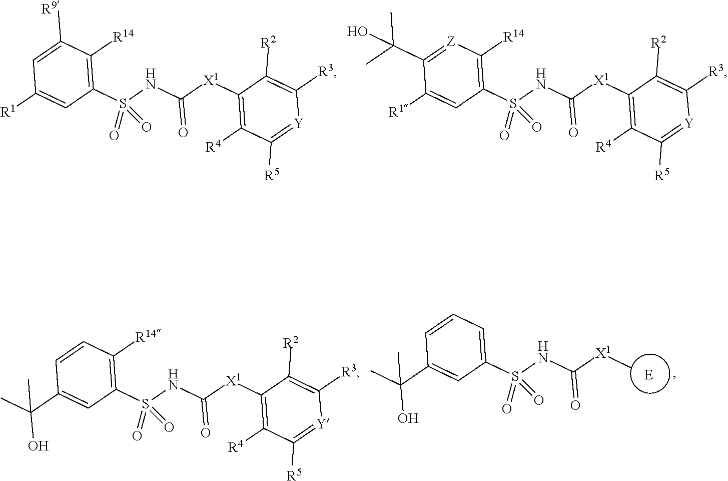

or a pharmaceutically acceptable salt thereof, wherein:

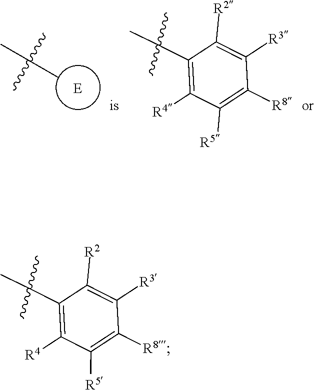

X¹ is NH;

Y is CR⁸;

Y′ is CR^{8′};

Z is N or CH;

R⁸ is selected from H, CN, Cl, F, and CONR¹¹R¹²;

R^{8′} is H;

R^{8″} is selected from CN or F;

R^{8′″} is selected from H, CN, CO₂C₁-C₆ alkyl, or CONR¹¹R¹²;

R² is C₁-C₆ alkyl;

R³ is hydrogen or halo;

R^{3′} is hydrogen or halo;

R⁴ is C₁-C₆ alkyl;

R⁵ is hydrogen;

R^{5′} is hydrogen;

provided that at least one of R², R³, R⁴ and R⁵ is not hydrogen, and that R² and R⁴ are not both hydroxymethyl;

or R² and R³, if both present, taken together with the carbons connecting them form a four-membered to seven-membered ring A,

or R⁴ and R⁵, if both present, taken together with the carbons connecting them form a four-membered to seven-membered ring B,

or R² and R³, if both present, taken together with the carbons connecting them form a four-membered to seven-membered ring A and R⁴ and R⁵, if both present, taken together with the carbons connecting them form a four-membered to seven-membered ring B,

R^{2″} and R^{3″}, if both present, taken together with the carbons connecting them form a four-membered to seven-membered ring A and R^{4″} and R^{5″}, if both present, taken together with the carbons connecting them form a four-membered to seven-membered ring B,

wherein ring A is

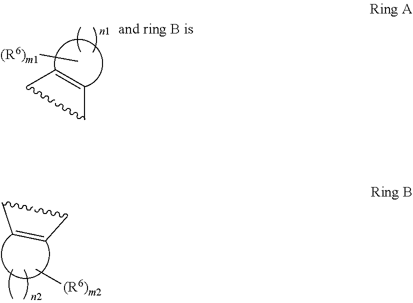

wherein

ring A is a carbocyclic ring;

n1 is 3;

m1 is 1;

wherein ring B is a carbocyclic ring;

n2 is 3;

m2 is 1;

wherein each R⁶ in each ring is the same or different and is selected from H;

R¹ is selected from CN, Cl, C(R¹⁰)₂OH, or F;

R^{1″} is selected from H or C(R¹)₂OH;

R¹⁴ is selected from H or F;

R^{14″} is F;

R^{9′} is selected from C(R¹)₂OH, C₃-C₆ cycloalkyl, phenyl, pyridyl, or morpholinyl each R¹⁰ is the same and is C₁-C₆ alkyl;

each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl.

In some embodiments of the compound of Formula IV, Formula IV is

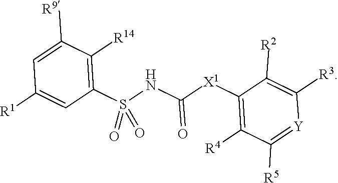

In some embodiments of the compound of Formula IV, R¹ is C(R¹⁰)₂OH.

In some embodiments of the compound of Formula IV, R¹ is CN.

In some embodiments of the compound of Formula IV, R¹ is C₁.

In some embodiments of the compound of Formula IV, R¹ is F.

In some embodiments of the compound of Formula IV, R^{9′} is pyridyl.

In some embodiments of the compound of Formula IV, R^{9′} is morpholinyl.

In some embodiments of the compound of Formula IV, R^{9′} is C₃-C₆ cycloalkyl.

In some embodiments of the compound of Formula IV, Formula IV is

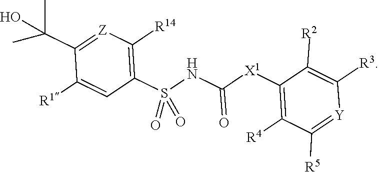

In some embodiments of the compound of Formula IV, Z is N.

In some embodiments of the compound of Formula IV, Z is CH.

In some embodiments of the compound of Formula IV, is H.

In some embodiments of the compound of Formula IV, Formula IV is

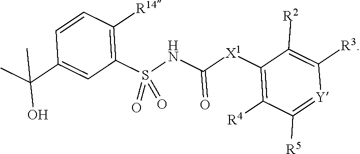

In some embodiments of the compound of Formula IV, R^{14″} is F.

In some embodiments of the compound of Formula IV, Formula IV is

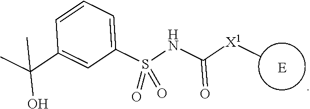

In some embodiments of the compound of Formula IV.

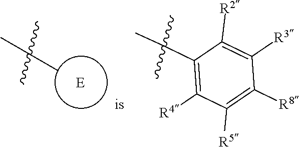

In some embodiments of the compound of Formula IV:
R^{2″} and R^{3″}, taken together with the carbons connecting them form a five membered ring A and R^{4″} and R^{5″}, taken together with the carbons connecting them form a five-membered ring B;
ring A is a saturated carbocyclic ring;
ring B is a saturated carbocyclic ring;
n1 is 3;
n2 is 3;
R⁶ is H.

In some embodiments of the compound of Formula IV, R^{8″} is F.

In some embodiments of the compound of Formula IV,

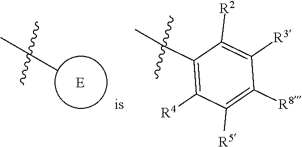

In some embodiments of the compound of Formula IV, R^{3′} is H or F, and R^{5′} is H.

In some embodiments of the compound of Formula IV, R^{8′″} is H.

In some embodiments of the compound of Formula IV, R^{8′″} is CN.

In some embodiments of the compound of Formula IV, R^{8′″} is CONH₂.

In some embodiments of the compound of Formula IV, R^{8′″} is COCH³.

In some embodiments of the compound of Formula IV, Formula IV is

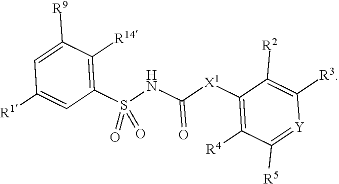

In some embodiments of the compound of Formula IV, R^{1′} is acetyl.

In some embodiments of the compound of Formula IV, R^{14′} is F.

In some embodiments of the compound of Formula IV, R⁹ is H.

In some embodiments of the compound of Formula IV, X¹ is NH.

In some embodiments of the compound of Formula IV, R² and R⁴ are each isopropyl.

In some embodiments of the compound of Formula IV, R³ and R⁵ are each H.

In some embodiments of the compound of Formula IV:
R² and R³, taken together with the carbons connecting them form a five membered ring A and R⁴ and R⁵, taken together with the carbons connecting them form a five-membered ring B;
ring A is a saturated carbocyclic ring;
ring B is a saturated carbocyclic ring;
n1 is 3;
n2 is 3;
R⁶ is H.

In some embodiments of the compound of Formula IV, Y is CR⁸.

In some embodiments of the compound of Formula IV, R⁸ is H.

In some embodiments of the compound of Formula IV, R⁸ is CN.

In some embodiments of the compound of Formula IV, R⁸ is C₁.

In some embodiments of the compound of Formula IV, R⁸ is F.

In some embodiments of the compound of Formula IV, R⁸ is CO₂NH₂.

In some embodiments of the compound of Formula IV, R⁸ is CO₂CH₃.

In another aspect, an NLRP3 antagonist is a compound selected from the group consisting of compounds in the Table 3 below and pharmaceutically acceptable salts thereof.

TABLE 3

| Com- | Structure | | |
|---|---|---|---|
| pound | | | |
| 101 | 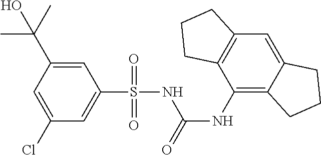 [SMILES: CC(C)(O)c1cc(Cl)cc(S(=O)(=O)NC(=O)Nc2c3c(cc4c2CCC4)CCC3)c1] | 102 | 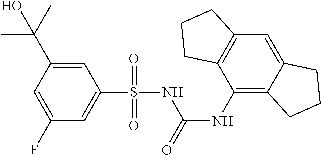 [SMILES: CC(C)(O)c1cc(F)cc(S(=O)(=O)NC(=O)Nc2c3c(cc4c2CCC4)CCC3)c1] |
| 103 | 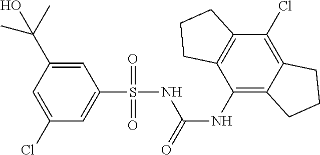 [SMILES: CC(C)(O)c1cc(Cl)cc(S(=O)(=O)NC(=O)Nc2c3c(c(Cl)c4c2CCC4)CCC3)c1] | 104 | 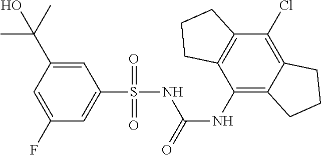 [SMILES: CC(C)(O)c1cc(F)cc(S(=O)(=O)NC(=O)Nc2c3c(c(Cl)c4c2CCC4)CCC3)c1] |
| 105 | 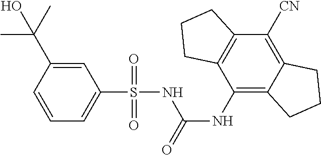 [SMILES: CC(C)(O)c1cccc(S(=O)(=O)NC(=O)Nc2c3c(c(C#N)c4c2CCC4)CCC3)c1] | 106 | 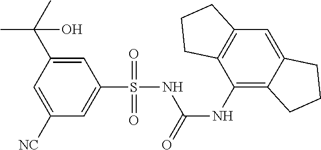 [SMILES: CC(C)(O)c1cc(C#N)cc(S(=O)(=O)NC(=O)Nc2c3c(cc4c2CCC4)CCC3)c1] |
| 107 | 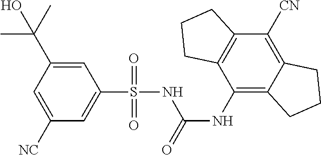 [SMILES: CC(C)(O)c1cc(C#N)cc(S(=O)(=O)NC(=O)Nc2c3c(c(C#N)c4c2CCC4)CCC3)c1] | 108 | 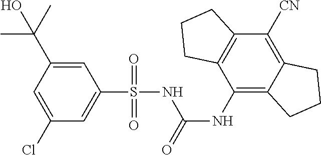 [SMILES: CC(C)(O)c1cc(Cl)cc(S(=O)(=O)NC(=O)Nc2c3c(c(C#N)c4c2CCC4)CCC3)c1] |
| 109 | 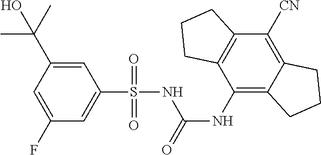 [SMILES: CC(C)(O)c1cc(F)cc(S(=O)(=O)NC(=O)Nc2c3c(c(C#N)c4c2CCC4)CCC3)c1] | 110 | 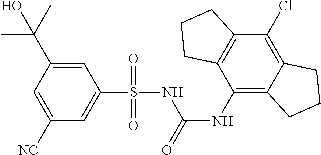 [SMILES: CC(C)(O)c1cc(C#N)cc(S(=O)(=O)NC(=O)Nc2c3c(c(Cl)c4c2CCC4)CCC3)c1] |
| 111 | 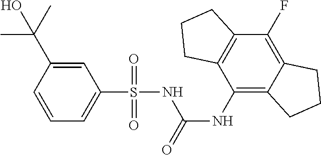 [SMILES: CC(C)(O)c1cccc(S(=O)(=O)NC(=O)Nc2c3c(c(F)c4c2CCC4)CCC3)c1] | 112 | 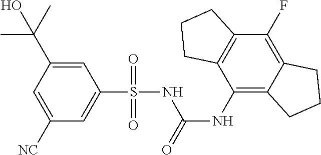 [SMILES: CC(C)(O)c1cc(C#N)cc(S(=O)(=O)NC(=O)Nc2c3c(c(F)c4c2CCC4)CCC3)c1] |
| 113 | 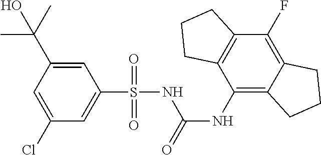 [SMILES: CC(C)(O)c1cc(Cl)cc(S(=O)(=O)NC(=O)Nc2c3c(c(F)c4c2CCC4)CCC3)c1] | 114 | 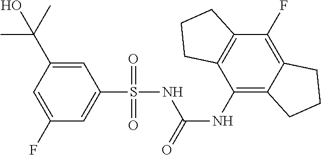 [SMILES: CC(C)(O)c1cc(F)cc(S(=O)(=O)NC(=O)Nc2c3c(c(F)c4c2CCC4)CCC3)c1] |
| 115 | 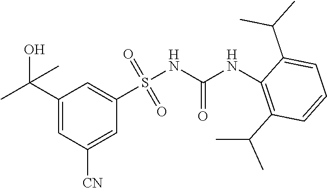 [SMILES: CC(C)c1cccc(C(C)C)c1NC(=O)NS(=O)(=O)c1cc(C#N)cc(C(C)(C)O)c1] | 116 | 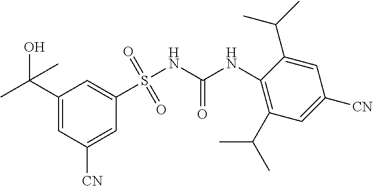 [SMILES: CC(C)c1cc(C#N)cc(C(C)C)c1NC(=O)NS(=O)(=O)c1cc(C#N)cc(C(C)(C)O)c1] |
| 117 | 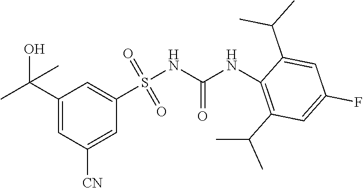 [SMILES: CC(C)c1cc(F)cc(C(C)C)c1NC(=O)NS(=O)(=O)c1cc(C#N)cc(C(C)(C)O)c1] | 118 | 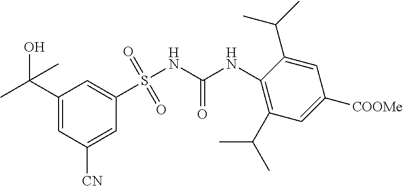 [SMILES: COC(=O)c1cc(C(C)C)c(NC(=O)NS(=O)(=O)c2cc(C#N)cc(C(C)(C)O)c2)c(C(C)C)c1] |
| 119 | 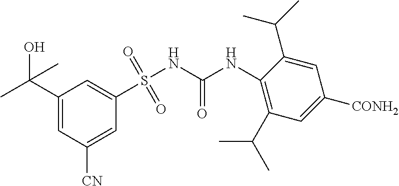 [SMILES: CC(C)c1cc(C(N)=O)cc(C(C)C)c1NC(=O)NS(=O)(=O)c1cc(C#N)cc(C(C)(C)O)c1] | 120 | 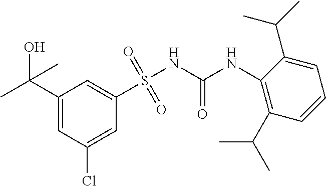 [SMILES: CC(C)c1cccc(C(C)C)c1NC(=O)NS(=O)(=O)c1cc(Cl)cc(C(C)(C)O)c1] |
| 121 | 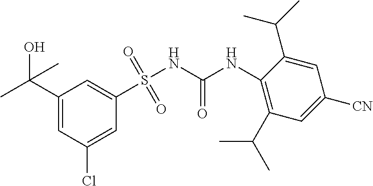 [SMILES: CC(C)c1cc(C#N)cc(C(C)C)c1NC(=O)NS(=O)(=O)c1cc(Cl)cc(C(C)(C)O)c1] | 122 | 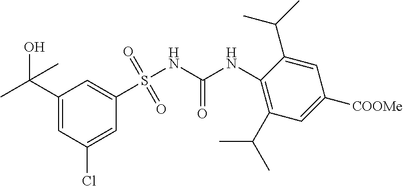 [SMILES: COC(=O)c1cc(C(C)C)c(NC(=O)NS(=O)(=O)c2cc(Cl)cc(C(C)(C)O)c2)c(C(C)C)c1] |
| 123 | 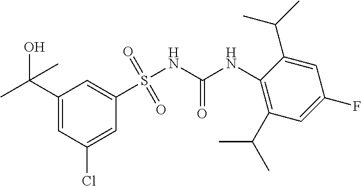 [SMILES: CC(C)c1cc(F)cc(C(C)C)c1NC(=O)NS(=O)(=O)c1cc(Cl)cc(C(C)(C)O)c1] | 124 | 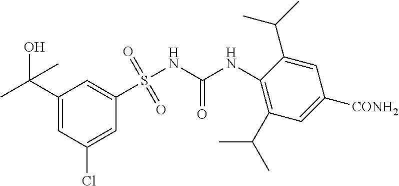 [SMILES: CC(C)c1cc(C(N)=O)cc(C(C)C)c1NC(=O)NS(=O)(=O)c1cc(Cl)cc(C(C)(C)O)c1] |
| 125 | 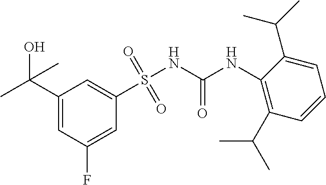 [SMILES: CC(C)c1cccc(C(C)C)c1NC(=O)NS(=O)(=O)c1cc(F)cc(C(C)(C)O)c1] | 126 | 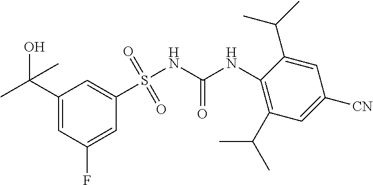 [SMILES: CC(C)c1cc(C#N)cc(C(C)C)c1NC(=O)NS(=O)(=O)c1cc(F)cc(C(C)(C)O)c1] |
| 127 | 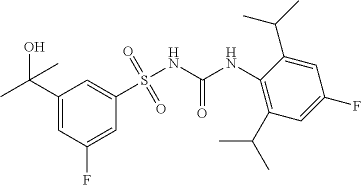 [SMILES: CC(C)c1cc(F)cc(C(C)C)c1NC(=O)NS(=O)(=O)c1cc(F)cc(C(C)(C)O)c1] | 128 | 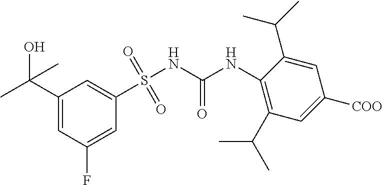 |
| 131 | 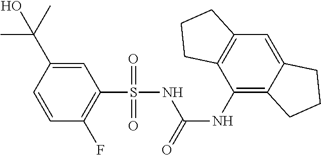 [SMILES: CC(C)(O)c1ccc(F)c(S(=O)(=O)NC(=O)Nc2c3c(cc4c2CCC4)CCC3)c1] | 132 | 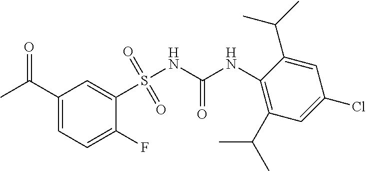 [SMILES: CC(=O)c1ccc(F)c(S(=O)(=O)NC(=O)Nc2c(C(C)C)cc(Cl)cc2C(C)C)c1] |
| 135 | 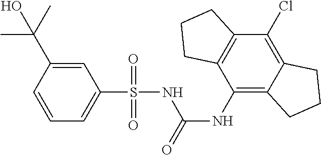 [SMILES: CC(C)(O)c1cccc(S(=O)(=O)NC(=O)Nc2c3c(c(Cl)c4c2CCC4)CCC3)c1] | 136 | 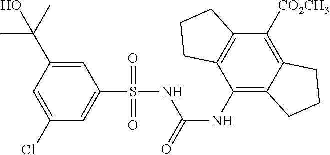 [SMILES: COC(=O)c1c2c(c(NC(=O)NS(=O)(=O)c3cc(Cl)cc(C(C)(C)O)c3)c3c1CCC3)CCC2] |

In another aspect, an NLRP3 antagonist is a compound selected from the group consisting of compounds in Table 4 below and pharmaceutically acceptable salts thereof.

TABLE 4

| Com- | Structure | | |
|---|---|---|---|
| pound | | | |
| 137 | 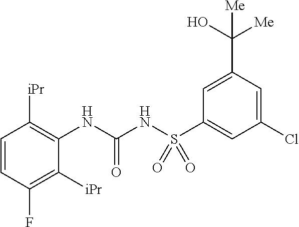 [SMILES: CC(C)c1ccc(F)c(C(C)C)c1NC(=O)NS(=O)(=O)c1cc(Cl)cc(C(C)(C)O)c1] | 138 | 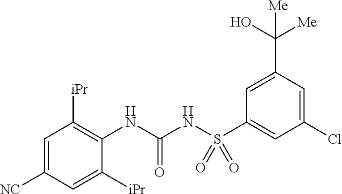 [SMILES: CC(C)c1cc(C#N)cc(C(C)C)c1NC(=O)NS(=O)(=O)c1cc(Cl)cc(C(C)(C)O)c1] |
| 141 | 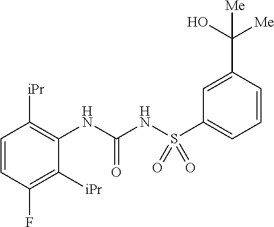 [SMILES: CC(C)c1ccc(F)c(C(C)C)c1NC(=O)NS(=O)(=O)c1cccc(C(C)(C)O)c1] | 142 | 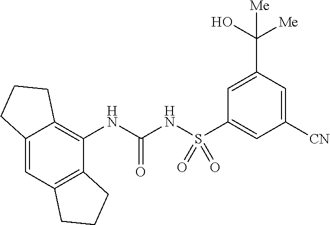 [SMILES: CC(C)(O)c1cc(C#N)cc(S(=O)(=O)NC(=O)Nc2c3c(cc4c2CCC4)CCC3)c1] |
| 143 | 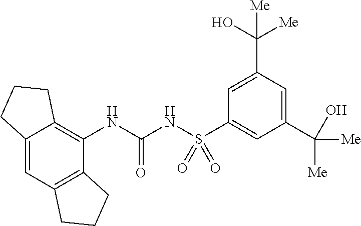 [SMILES: CC(C)(O)c1cc(C(C)(C)O)cc(S(=O)(=O)NC(=O)Nc2c3c(cc4c2CCC4)CCC3)c1] | 144 | 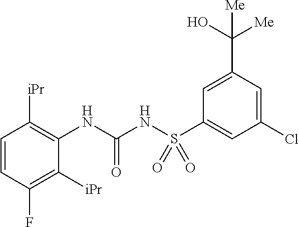 [SMILES: CC(C)c1ccc(F)c(C(C)C)c1NC(=O)NS(=O)(=O)c1cc(Cl)cc(C(C)(C)O)c1] |
| 145 | 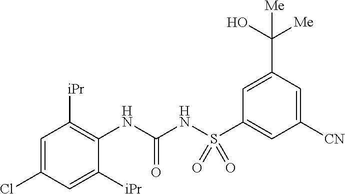 [SMILES: CC(C)c1cc(Cl)cc(C(C)C)c1NC(=O)NS(=O)(=O)c1cc(C#N)cc(C(C)(C)O)c1] | 146 | 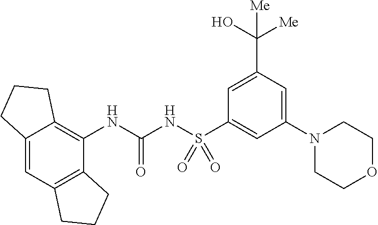 [SMILES: CC(C)(O)c1cc(N2CCOCC2)cc(S(=O)(=O)NC(=O)Nc2c3c(cc4c2CCC4)CCC3)c1] |
| 147 | 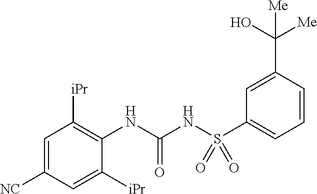 [SMILES: CC(C)c1cc(C#N)cc(C(C)C)c1NC(=O)NS(=O)(=O)c1cccc(C(C)(C)O)c1] | 148 | 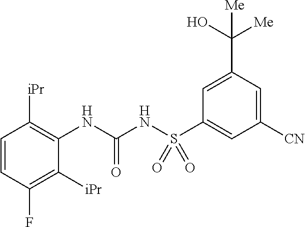 [SMILES: CC(C)c1ccc(F)c(C(C)C)c1NC(=O)NS(=O)(=O)c1cc(C#N)cc(C(C)(C)O)c1] |
| 149 | 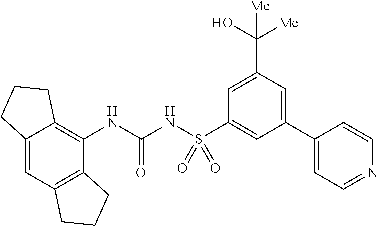 [SMILES: CC(C)(O)c1cc(-c2ccncc2)cc(S(=O)(=O)NC(=O)Nc2c3c(cc4c2CCC4)CCC3)c1] | 150 | 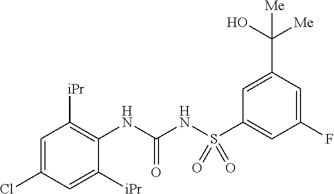 [SMILES: CC(C)c1cc(Cl)cc(C(C)C)c1NC(=O)NS(=O)(=O)c1cc(F)cc(C(C)(C)O)c1] |
| 151 | 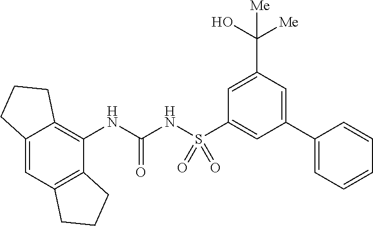 [SMILES: CC(C)(O)c1cc(-c2ccccc2)cc(S(=O)(=O)NC(=O)Nc2c3c(cc4c2CCC4)CCC3)c1] | 153 | 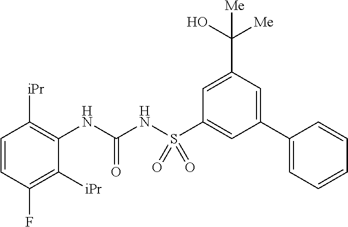 [SMILES: CC(C)c1ccc(F)c(C(C)C)c1NC(=O)NS(=O)(=O)c1cc(-c2ccccc2)cc(C(C)(C)O)c1] |
| 154 | 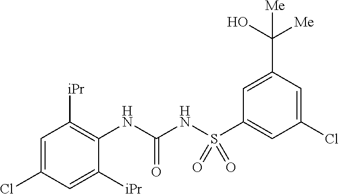 [SMILES: CC(C)c1cc(Cl)cc(C(C)C)c1NC(=O)NS(=O)(=O)c1cc(Cl)cc(C(C)(C)O)c1] | 155 | 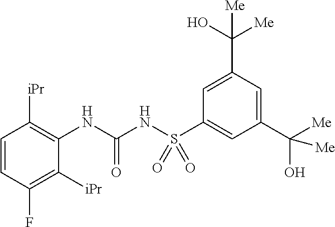 [SMILES: CC(C)c1ccc(F)c(C(C)C)c1NC(=O)NS(=O)(=O)c1cc(C(C)(C)O)cc(C(C)(C)O)c1] |
| 157 | 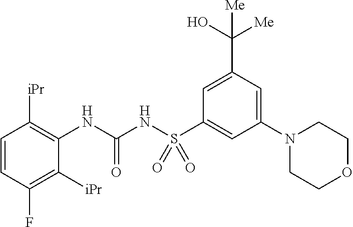 [SMILES: CC(C)c1ccc(F)c(C(C)C)c1NC(=O)NS(=O)(=O)c1cc(N2CCOCC2)cc(C(C)(C)O)c1] | 158 | 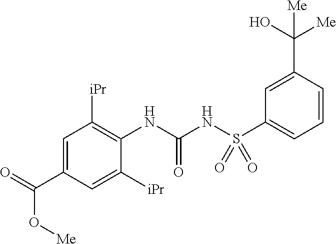 [SMILES: COC(=O)c1cc(C(C)C)c(NC(=O)NS(=O)(=O)c2cccc(C(C)(C)O)c2)c(C(C)C)c1] |
| 159 | 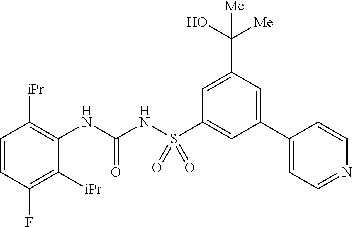 [SMILES: CC(C)c1ccc(F)c(C(C)C)c1NC(=O)NS(=O)(=O)c1cc(-c2ccncc2)cc(C(C)(C)O)c1] | 160 | 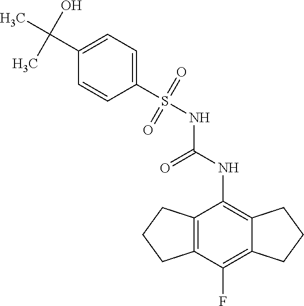 [SMILES: CC(C)(O)c1ccc(S(=O)(=O)NC(=O)Nc2c3c(c(F)c4c2CCC4)CCC3)cc1] |
| 161 | 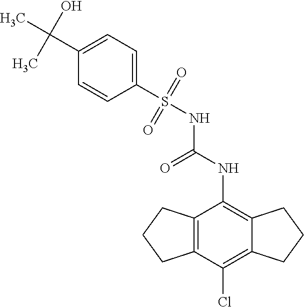 [SMILES: CC(C)(O)c1ccc(S(=O)(=O)NC(=O)Nc2c3c(c(Cl)c4c2CCC4)CCC3)cc1] | 162 | 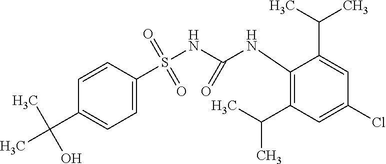 [SMILES: CC(C)c1cc(Cl)cc(C(C)C)c1NC(=O)NS(=O)(=O)c1ccc(C(C)(C)O)cc1] |
| 163 | 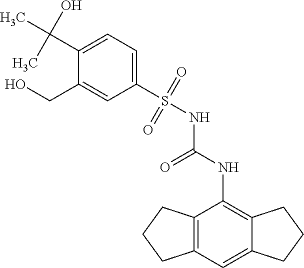 [SMILES: CC(C)(O)c1ccc(S(=O)(=O)NC(=O)Nc2c3c(cc4c2CCC4)CCC3)cc1CO] | 164 | 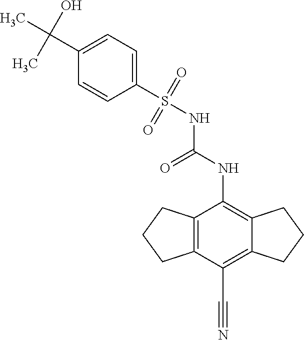 [SMILES: CC(C)(O)c1ccc(S(=O)(=O)NC(=O)Nc2c3c(c(C#N)c4c2CCC4)CCC3)cc1] |
| 165 | 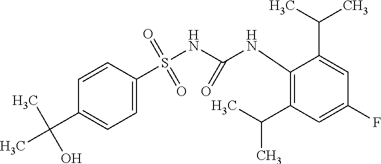 [SMILES: CC(C)c1cc(F)cc(C(C)C)c1NC(=O)NS(=O)(=O)c1ccc(C(C)(C)O)cc1] | 166 | 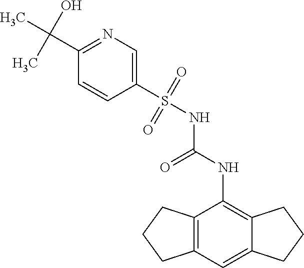 [SMILES: CC(C)(O)c1ccc(S(=O)(=O)NC(=O)Nc2c3c(cc4c2CCC4)CCC3)cn1] |
| 167 | 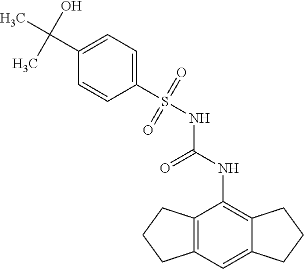 [SMILES: CC(C)(O)c1ccc(S(=O)(=O)NC(=O)Nc2c3c(cc4c2CCC4)CCC3)cc1] | 168 | 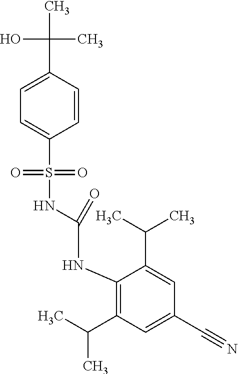 [SMILES: CC(C)c1cc(C#N)cc(C(C)C)c1NC(=O)NS(=O)(=O)c1ccc(C(C)(C)O)cc1] |
| 169 | 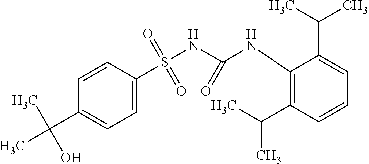 [SMILES: CC(C)c1cccc(C(C)C)c1NC(=O)NS(=O)(=O)c1ccc(C(C)(C)O)cc1] | 170 | 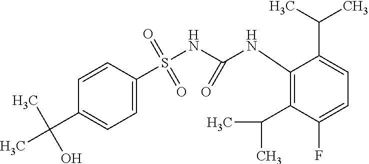 [SMILES: CC(C)c1ccc(F)c(C(C)C)c1NC(=O)NS(=O)(=O)c1ccc(C(C)(C)O)cc1] |
| 171 | 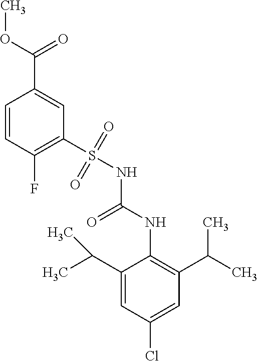 [SMILES: COC(=O)c1ccc(F)c(S(=O)(=O)NC(=O)Nc2c(C(C)C)cc(Cl)cc2C(C)C)c1] | | |

Compounds having Formula III and IV, as well as methods of making and using the same, are further described in WO2017184623A1 (PCT/US2017/028166), filed on Apr. 18, 2017; U.S. Provisional 62/324,071, filed on Apr. 18, 2016; and U.S. Provisional 62/324,081, filed on Apr. 18, 2016, each of which is incorporated herein by reference in its entirety.

In one aspect, an NLRP3 antagonist is a compound of Formula V

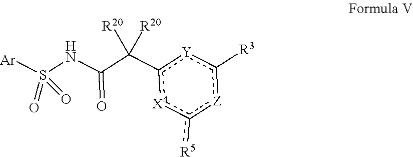

or a pharmaceutically acceptable salt thereof, wherein:

Ar is a heteroaryl group

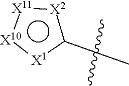

or an aryl or heteroaryl group

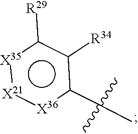

X¹ is O, S, N, CR⁴¹ or NR⁴¹;

X¹⁰ is O, S, N, CR¹⁰ or NR¹⁰;

X¹¹ is O, S, N, CR¹ or NR¹;

X² is O, S, N, CR⁴² or NR⁴²;

X³⁵ is N or CR³⁵;

X²¹ is N or CR²¹;

X³⁶ is N or CR³⁶;

X⁴ is CR⁴, N or NR²⁴;

each R²⁰ is the same or different and is independently selected from hydrogen and C₁-C₆ alkyl;

Y is N or CR²;

Z is N or CR⁸;

R⁸ is selected from H, CN, halo CO₂C₁-C₆ alkyl, CO₂C₃-C₈ cycloalkyl, CONR¹¹R¹², C₁-C₆ alkyl, C₁-C₆ alkoxy, C₁-C₆ haloalkoxy, and C₁-C₆ haloalkyl;

R² is hydrogen, C₁-C₆ alkoxy, halo, C₁-C₆ haloalkyl, C₃-C₇ cycloalkyl or C₁-C₆ alkyl optionally substituted with hydroxy;

R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;

R⁴ is hydrogen, C₁-C₆ alkoxy, halo, C₁-C₆ haloalkyl, C₃-C₇ cycloalkyl or C₁-C₆ alkyl optionally substituted with hydroxy;

R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;

or R²⁴ is C₁-C₆ alkyl or C₃-C₈ cycloalkyl and R⁵ is ═O;

provided that at least one of R², R³, R⁴ and R⁵ is not hydrogen;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

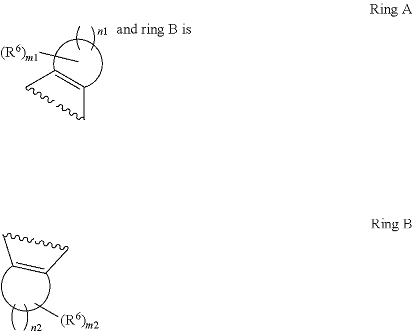

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, F, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S; each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;

and each of R1R¹⁰, R⁴¹ and, R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ akyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;
or R¹ and R¹⁰ taken together with the atoms connecting them form a 3-to-8-membered carbocyclic or heterocyclic ring containing 1 or 2 heteroatoms independently selected from O, N, and S, wherein the ring is optionally substituted with one or more substituents each independently selected from hydroxy, oxo, C₁-C₆ alkoxy, NR¹¹R¹²═NR¹³, COOC₁-C₆ alkyl, and CONR¹¹R¹²;
each of R³⁴, R²⁹, R³⁵, R²¹ and R³⁶ is independently selected from H, C₁-C₆ alkyl,

C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl, SF₅ and S(O₂)C₁-C₆ akyl;

wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, C₁-C₆ alkyl, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO (5- to 10-membered heteroaryl), NHCO (3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl,

or two groups selected from R³⁴, R²⁹, R³⁵, R²¹ and R³⁶ that are on adjacent ring carbon atoms taken together with the adjacent ring carbons form a 6-membered aromatic ring, a five-to-eight-membered carbocyclic non-aromatic ring, a five- or six-membered heteroaromatic ring or a five-to-eight-membered heterocyclic non-aromatic ring, wherein the ring formed by the two groups together with the adjacent ring carbons is optionally substituted with one or more OC₁-C₆ alkyl, NH₂, NHC₁-C₆ alkyl, N(C₁-C₆ alkyl)₂;

R¹³ is C₁-C₆ alkyl;

each of R¹¹ and R¹² at each occurrence is independently selected from hydrogen, C₁-C₆ alkyl, CO₂R¹⁵ and CONR¹⁷R¹⁸; or R¹¹ and R¹² taken together with the nitrogen they are attached to form a 3- to 7-membered ring optionally containing one or more heteroatoms in addition to the nitrogen they are attached to;

R¹⁵ is C₁-C₆ alkyl;

each of R¹⁷ and R¹⁸ at each occurrence is independently selected from hydrogen and C₁-C₆ alkyl.

In another aspect, an NLRP3 antagonist is a compound of Formula V-I

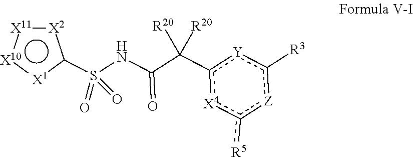

or a pharmaceutically acceptable salt thereof, wherein:

X¹ is O, S, N, CR⁴¹ or NR⁴¹;

X¹⁰ is O, S, N, CR¹⁰ or NR¹⁰;

X¹¹ is O, S, N, CR¹ or NR¹;

X² is O, S, N, CR⁴² or NR⁴²;

X⁴ is CR⁴, N or NR²⁴;
each R²⁰ is the same or different and is independently selected from hydrogen and C₁-C₆ alkyl;

Y is N or CR²;

Z is N or C⁸;

R⁸ is selected from H, CN, halo, CO₂C₁-C₆ alkyl, CO₂C₃-C₈ cycloalkyl, CONR¹¹R¹², C₁-C₆ alkyl, C₁-C₆ alkoxy, C₁-C₆ haloalkoxy, and C₁-C₆ haloalkyl;

R² is hydrogen, C₁-C₆ alkoxy, halo, C₁-C₆ haloalkyl, C₃-C₇ cycloalkyl or C₁-C₆ alkyl optionally substituted with hydroxy;

R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;

R⁴ is hydrogen, C₁-C₆ alkoxy, halo, C₁-C₆ haloalkyl, C₃-C₇ cycloalkyl or C₁-C₆ alkyl optionally substituted with hydroxy;

R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;

or R²⁴ is C₁-C₆ alkyl or C₃-C₈ cycloalkyl and R⁵ is ═O;

provided that at least one of R², R³, R⁴ and R⁵ is not hydrogen;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

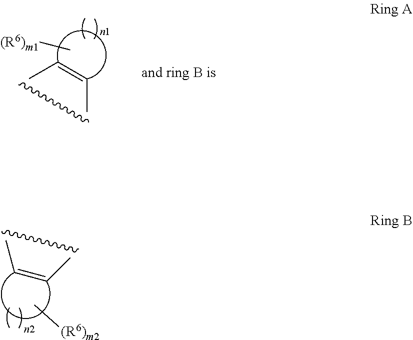

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, F, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ akyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkyl, C₁-C₆ alkoxy, NR¹¹R¹², ═NR¹³, COOC₁-C₆ alkyl, C₆-C₁₀ aryl, and CONR¹¹R¹²;

and each of R¹, R¹⁰, R⁴¹ and R⁴², when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl, S(O₂)C₁-C₆ akyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, C₁-C₆ alkyl, oxo, C₁-C₆ alkoxy, NR¹¹R¹²; —NR¹³; COOC₁-C₆ alkyl, C₆-C₁₀ aryl, and CONR¹¹R¹²;

or R¹ and R¹⁰ taken together with the atoms connecting them form a 3-to-8-membered carbocyclic or heterocyclic ring containing 1 or 2 heteroatoms independently selected from O, N, and S, wherein the ring is optionally substituted with one or more substituents each independently selected from hydroxy, oxo, C₁-C₆ alkoxy, NR¹¹R¹² ═NR¹³, COOC₁-C₆ alkyl, and CONR¹¹R¹²;

R¹³ is C₁-C₆ alkyl;

each of R¹¹ and R¹² at each occurrence is independently selected from hydrogen,

C₁-C₆ alkyl, CO₂R¹⁵ and CONR¹⁷R¹⁸; or R¹¹ and R¹² taken together with the nitrogen they are attached to form a 3- to 7-membered ring optionally containing one or more heteroatoms in addition to the nitrogen they are attached to;

R¹⁵ is C₁-C₆ alkyl;

each of R¹⁷ and R¹⁸ at each occurrence is independently selected from hydrogen and C₁-C₆ alkyl.

In another aspect, an NLRP3 antagonist is a compound of Formula V-II

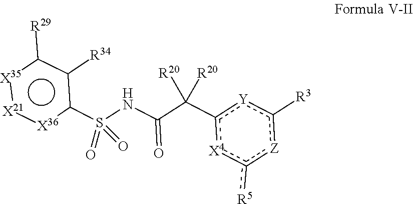

or a pharmaceutically acceptable salt thereof, wherein:

X³⁵ is N or CR³⁵;

X²¹ is N or CR²¹;

X³⁶ is N or CR³⁶;

X⁴ is CR⁴, N or NR²⁴;

each R²⁰ is the same or different and is independently selected from hydrogen and C₁-C₆ alkyl;

Y is N or CR²;

Z is N or CR⁸;

R⁸ is selected from H, CN, halo, CO₂C₁-C₆ alkyl, CO₂C₃-C₈ cycloalkyl, CONR¹¹R¹², C₁-C₆ alkyl, C₁-C₆ alkoxy, C₁-C₆ haloalkoxy, and C₁-C₆ haloalkyl;

R² is hydrogen, C₁-C₆ alkoxy, halo, C₁-C₆ haloalkyl, C₃-C₇ cycloalkyl or C₁-C₆ alkyl optionally substituted with hydroxy;

R³ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;

R⁴ is hydrogen, C₁-C₆ alkoxy, halo, C₁-C₆ haloalkyl, C₃-C₇ cycloalkyl or C₁-C₆ alkyl optionally substituted with hydroxy;

R²⁴ is absent and R⁵ is hydrogen, C₁-C₆ alkoxy, halo, C₁-C₆ haloalkyl, CN, C₁-C₆ haloalkoxy, C₃-C₇ cycloalkyl or C₁-C₆ alkyl optionally substituted with hydroxy;

or R²⁴ is C₁-C₆ alkyl or C₃-C₈ cycloalkyl and R⁵ is ═O;

provided that at least one of R², R³, R⁴ and R⁵ is not hydrogen;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

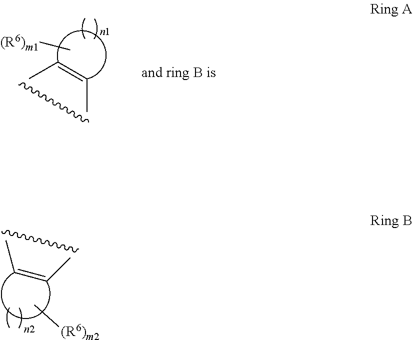

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, F, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl, SF₅ and S(O₂)C₁-C₆ akyl;

wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, C₁-C₆ alkyl, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO (5- to 10-membered heteroaryl), NHCO (3- to 7-membered heterocycloalkyl), and NHCOC₂-C₆ alkynyl,

wherein the C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl,

or two groups selected from R³⁴, R²⁹, R³⁵, R²¹ and R³⁶ that are on adjacent ring carbon atoms taken together with the adjacent ring carbons form a 6-membered aromatic ring, a five-to-eight-membered carbocyclic non-aromatic ring, a five- or six-membered heteroaromatic ring or a five-to-eight-membered heterocyclic non-aromatic ring, wherein the ring formed by the two groups together with the adjacent ring carbons is optionally substituted with one or more OC₁-C₆ alkyl, NH₂, NHC₁-C₆ alkyl, N(C₁-C₆ alkyl)₂;

R¹³ is C₁-C₆ alkyl;

each of R¹¹ and R¹² at each occurrence is independently selected from hydrogen, C₁-C₆ alkyl, CO₂R¹⁵ and CONR¹⁷R¹⁸; or R¹¹ and R¹² taken together with the nitrogen they are attached to form a 3- to 7-membered ring optionally containing one or more heteroatoms in addition to the nitrogen they are attached to;

R¹⁵ is C₁-C₆ alkyl;

each of R¹⁷ and R¹⁸ at each occurrence is independently selected from hydrogen and C₁-C₆ alkyl.

In another aspect, an NLRP3 antagonist is a compound of Formula V

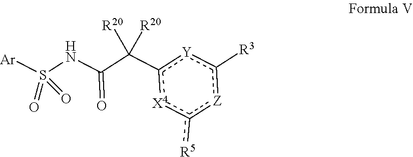

or a pharmaceutically acceptable salt thereof, wherein:

Ar is a heteroaryl group

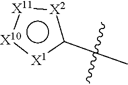

or an aryl or heteroaryl group

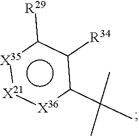

X¹ is O, S, N, CR⁴¹ or NR⁴¹;

X¹⁰ is O, S, N, CR¹⁰ or NR¹⁰;

X¹¹ is O, S, N, CR¹ or NR¹;

X² is O, S, N, CR⁴² or NR⁴²;

X³⁵ is N or CR³⁵;

X²¹ is N or CR²¹;

X³⁶ is N or CR³⁶;

X⁴ is CR⁴, N or NR²⁴;

each R²⁰ is the same or different and is independently selected from hydrogen and C₁-C₆ alkyl;

Y is N or CR²;

Z is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, C₃-C₇ cycloalkyl or C₁-C₆ alkyl optionally substituted with hydroxy;

R³ is hydrogen, C₁-C₆ alkoxy, halo, C₁-C₆ haloalkyl, C₃-C₇ cycloalkyl or C₁-C₆ alkyl optionally substituted with hydroxy;

R⁴ is hydrogen, C₁-C₆ alkoxy, halo, C₁-C₆ haloalkyl, C₃-C₇ cycloalkyl or C₁-C₆ alkyl optionally substituted with hydroxy;

R²⁴ is absent and R⁵ is hydrogen, C₁-C₆ alkoxy, halo, C₁-C₆ haloalkyl, C₃-C₇ cycloalkyl or C₁-C₆ alkyl optionally substituted with hydroxy;

or R²⁴ is C₁-C₆ alkyl or C₃-C₈ cycloalkyl and R⁵ is ═O;

provided that at least one of R², R³, R⁴ and R⁵ is not hydrogen, and that R² and R⁴ are not both hydroxymethyl;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

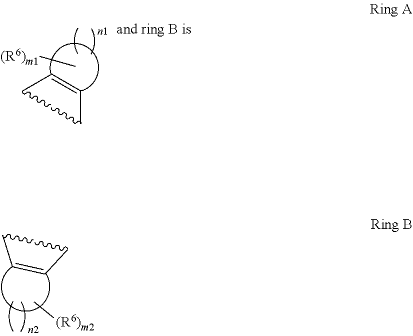

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, C₆-C₁₀ aryl, and CONR¹¹R¹²;

and each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, C₆-C₁₀ aryl, and CONR¹¹R¹²;

or R¹ and R¹⁰ taken together with the atoms connecting them form a 3-to-8-membered carbocyclic or heterocyclic ring containing 1 or 2 heteroatoms independently selected from O, N, and S, wherein the ring is optionally substituted with one or more substituents each independently selected from hydroxy, oxo, C₁-C₆ alkoxy, NR¹¹R¹², ═NR¹³, COOC₁-C₆ alkyl, and CONR¹¹R¹²;

each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl,

wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO (5- to 10-membered heteroaryl), NHCO (3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl,

or two groups selected from R³⁴, R²⁹, R³⁵, R²¹ and R³⁶ that are on adjacent ring carbon atoms taken together with the adjacent ring carbons form a 6-membered aromatic ring, a five-to-eight-membered carbocyclic non-aromatic ring, a five- or six-membered heteroaromatic ring or a five-to-eight-membered heterocyclic non-aromatic ring, wherein the ring formed by the two groups together with the adjacent ring carbons is optionally substituted with one or more OC₁-C₆ alkyl, NH₂, NHC₁-C₆ alkyl, N(C₁-C₆ alkyl)₂;

R³ is C₁-C₆ alkyl;

each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl.

In another aspect, an NLRP3 antagonist is a compound of Formula V-I

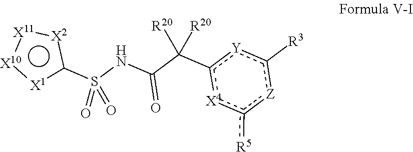

or a pharmaceutically acceptable salt thereof, wherein:

X¹ is O, S, N, CR′ or NR⁴¹;

X¹⁰ is O, S, N, CR¹⁰ or NR¹⁰;

X¹¹ is O, S, N, CR¹ or NR¹;

X² is O, S, N, CR⁴² or NR⁴²;

X⁴ is CR⁴, N or NR²⁴;

each R²⁰ is the same or different and is independently selected from hydrogen and C₁-C₆ alkyl;

Y is N or CR²;

Z is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, C₃-C₇ cycloalkyl or C₁-C₆ alkyl optionally substituted with hydroxy;

R³ is hydrogen, C₁-C₆ alkoxy, halo, C₁-C₆ haloalkyl, C₃-C₇ cycloalkyl or C₁-C₆ alkyl optionally substituted with hydroxy;

R⁴ is hydrogen, C₁-C₆ alkoxy, halo, C₁-C₆ haloalkyl, C₃-C₇ cycloalkyl or C₁-C₆ alkyl optionally substituted with hydroxy;

R²⁴ is absent and R⁵ is hydrogen, C₁-C₆ alkoxy, halo, C₁-C₆ haloalkyl, C₃-C₇ cycloalkyl or C₁-C₆ alkyl optionally substituted with hydroxy;

or R²⁴ is C₁-C₆ alkyl or C₃-C₈ cycloalkyl and R⁵ is ═O;

provided that at least one of R², R³, R⁴ and R⁵ is not hydrogen, and that R² and R⁴ are not both hydroxymethyl;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

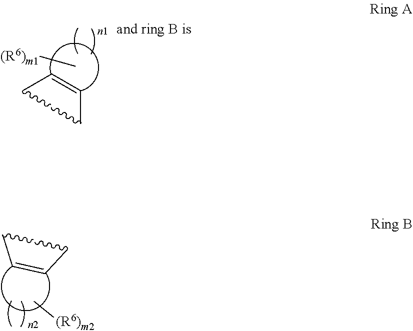

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

each of R¹, R¹⁰, R⁴¹ and R⁴² when bonded to carbon is independently selected from H, C₁-C₆ alkyl, C₁-C₆ haloalkyl, CN, halo, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, C₆-C₁₀ aryl, and CONR¹¹R¹²;

and each of R¹, R¹⁰, R⁴¹ and R⁴² when bonded to nitrogen is independently selected from H, C₁-C₆ alkyl, C₁-C₆ haloalkyl, CO₂C₁-C₆ alkyl, CO₂C₃-C₈ cycloalkyl, C₆-C₁₀ aryl, CONR¹¹R¹², C₃-C₇ cycloalkyl and 3- to 7-membered heterocycloalkyl, wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, C₆-C₁₀ aryl, and CONR¹¹R¹²;

or R¹ and R¹⁰ taken together with the atoms connecting them form a 3-to-8-membered carbocyclic or heterocyclic ring containing 1 or 2 heteroatoms independently selected from O, N, and S, wherein the ring is optionally substituted with one or more substituents each independently selected from hydroxy, oxo, C₁-C₆ alkoxy, NR¹¹R¹², ═NR¹³, COOC₁-C₆ alkyl, and CONR¹¹R¹²;

R¹³ is C₁-C₆ alkyl;

each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl.

In another aspect, an NLRP3 antagonist is a compound of Formula V-II

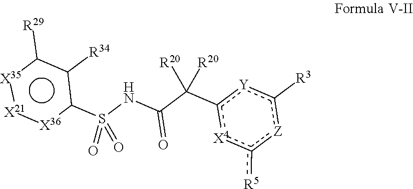

or a pharmaceutically acceptable salt thereof, wherein:

X³⁵ is N or CR³⁵;

X²¹ is N or CR²¹;

X³⁶ is N or CR³⁶;

X⁴ is CR⁴, N or NR²⁴;
each R²⁰ is the same or different and is independently selected from hydrogen and C₁-C₆ alkyl;

Y is N or CR²;

Z is N or CR⁸;

R⁸ is selected from H, CN, Cl, F, CO₂C₁-C₆ alkyl, CO₂C₃-C₈ cycloalkyl, CONR¹¹R¹², C₁-C₆ alkyl, and C₁-C₆ haloalkyl;

R² is hydrogen, C₁-C₆ alkoxy, halo, C₁-C₆ haloalkyl, C₃-C₇ cycloalkyl or C₁-C₆ alkyl optionally substituted with hydroxy;

R³ is hydrogen, C₁-C₆ alkoxy, halo, C₁-C₆ haloalkyl, C₃-C₇ cycloalkyl or C₁-C₆ alkyl optionally substituted with hydroxy;

R⁴ is hydrogen, C₁-C₆ alkoxy, halo, C₁-C₆ haloalkyl, C₃-C₇ cycloalkyl or C₁-C₆ alkyl optionally substituted with hydroxy;

R²⁴ is absent and R⁵ is hydrogen, C₁-C₆ alkoxy, halo, C₁-C₆ haloalkyl, C₃-C₇ cycloalkyl or C₁-C₆ alkyl optionally substituted with hydroxy;

or R²⁴ is C₁-C₆ alkyl or C₃-C₈ cycloalkyl and R⁵ is ═O;

provided that at least one of R², R³, R⁴ and R⁵ is not hydrogen, and that R² and R⁴ are not both hydroxymethyl;

or R² and R³ taken together with the carbons connecting them form a four-membered to seven-membered ring A,

or R⁴ and R⁵ taken together with the carbons connecting them form a four-membered to seven-membered ring B,

or R² and R³ taken together with the carbons connecting them form a four-membered to seven-membered ring A and R⁴ and R⁵ taken together with the carbons connecting them form a four-membered to seven-membered ring B,

wherein ring A is

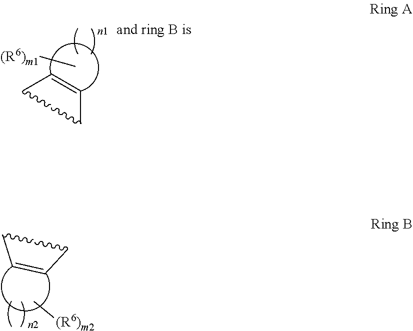

wherein

ring A is a carbocyclic ring or a heterocyclic ring containing 1 or 2 heteroatoms independently selected from O, N, and S;

n1 is from 2 to 5;

m1 is from 1 to 10;

wherein ring B is a carbocyclic ring or a heterocyclic ring containing 1 or 2 heteroatoms independently selected from O, N, and S;

n2 is from 2 to 5;

m2 is from 1 to 10;

wherein each R⁶ in each ring is the same or different and is selected from H, C₁-C₆ alkyl, C₁-C₆ alkoxy, NR¹¹R¹², oxo, and ═NR¹³;

or two R⁶ taken together with the atom or atoms connecting them form a 3-to-8-membered carbocyclic or saturated heterocyclic ring containing 1 or 2 heteroatoms independently selected from O, N, and S;

each of R³⁴, R²⁹, R³⁵, R²¹ and R³⁶ is independently selected from H, C₁-C₆ alkyl, C₁-C₆ haloalkyl, CN, halo, CO₂C₁-C₆ alkyl, CO₂C₃-C₈ cycloalkyl, CONR¹¹R¹², C₃-C₇ cycloalkyl, 3- to 7-membered heterocycloalkyl, C₆-C₁₀ aryl, OC₁-C₆ alkyl, NH₂, NHC₁-C₆ alkyl, N(C₁-C₆ alkyl)₂, NO₂, COC₁-C₆ alkyl,

wherein the C₁-C₆ alkyl, C₃-C₇ cycloalkyl and 3- to 7-membered heterocycloalkyl is optionally substituted with one or more substituents each independently selected from hydroxy, oxo, C₁-C₆ alkoxy, NR¹¹R¹², ═NR¹³, COOC₁-C₆ alkyl, CONR¹¹R¹², C₃-C₇ cycloalkyl, 3- to 7-membered heterocycloalkyl, C₆-C₁₀ aryl, 5- to 10-membered heteroaryl, NHCOC₆-C₁₀ aryl, NHCO (5- to 10-membered heteroaryl), NHCO (3- to 7-membered heterocycloalkyl), and NHCOC₂-C₆ alkynyl,
wherein the C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl,

or two groups selected from R³⁴, R²⁹, R³⁵, R²¹ and R³⁶ that are on adjacent ring carbon atoms taken together with the adjacent ring carbons form a 6-membered aromatic ring, a five-to-eight-membered carbocyclic non-aromatic ring, a five- or six-membered heteroaromatic ring or a five-to-eight-membered heterocyclic non-aromatic ring, wherein the ring formed by the two groups together with the adjacent ring carbons is optionally substituted with one or more OC₁-C₆ alkyl, NH₂, NHC₁-C₆ alkyl, N(C₁-C₆ alkyl)₂;

R¹³ is C₁-C₆ alkyl;

each of R¹¹ and R¹² at each occurrence is independently selected from hydrogen, C₁-C₆ alkyl, CO₂R¹⁵ and CONR¹⁷R¹⁸;

R¹⁵ is C₁-C₆ alkyl;

each of R¹⁷ and R¹⁸ at each occurrence is independently selected from hydrogen and C₁-C₆ alkyl.

In some embodiments of the compound of Formula V, the moiety

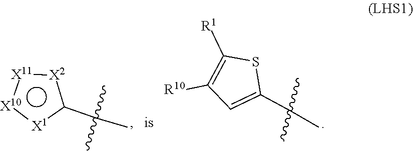

In some embodiments of the compound of Formula V, the moiety

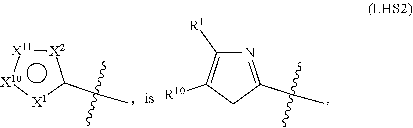

In some embodiments of the compound of Formula V, the moiety

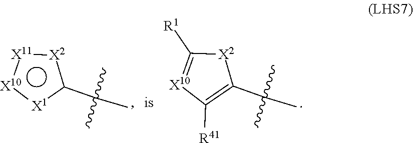

In certain of the foregoing embodiments of Formula V, X¹⁰ is N; and X² is S.

In certain of the foregoing embodiments of Formula V, LHS7 is

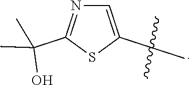 [SMILES: *c1cnc(C(C)(C)O)s1]

In some embodiments of the compound of Formula V, the moiety

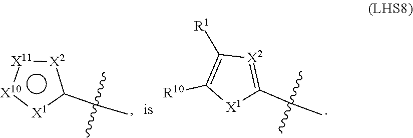

In some embodiments of the compound of Formula V, X¹ is S; and X² is CH.

In some embodiments of the compound of Formula V, the moiety

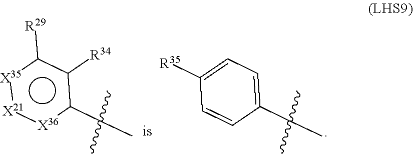

In some embodiments of the compound of Formula V, the moiety

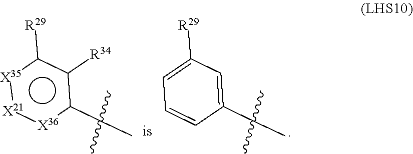

In some embodiments of the compound of Formula V, the moiety

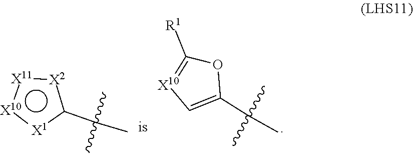

In some embodiments of the compound of Formula V, the moiety

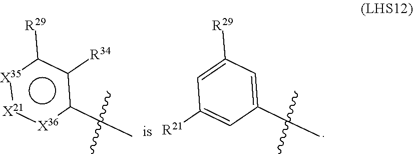

In some embodiments of the compound of Formula V, the moiety

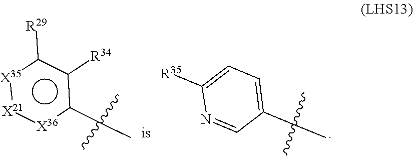

In some embodiments of the compound of Formula V, the moiety

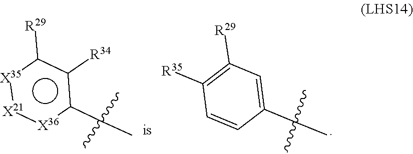

In some embodiments of the compound of Formula V, the moiety

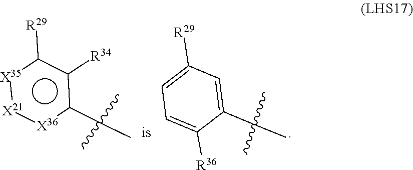

In some embodiments of the compound of Formula V, LHS17 is

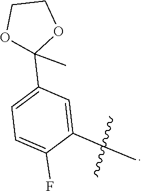 [SMILES: *c1cc(C2(C)OCCO2)ccc1F]

In some embodiments of the compound of Formula V, the moiety

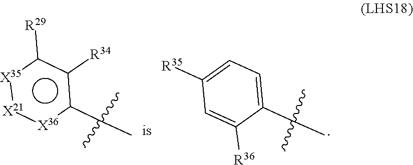

In some embodiments of the compound of Formula V, the moiety

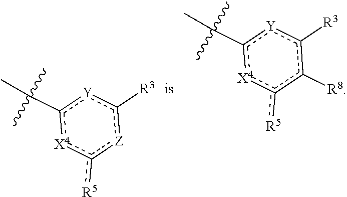

In certain of the foregoing embodiments of Formula V (when the moiety

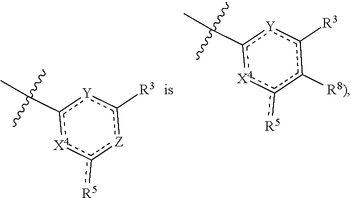

the moiety

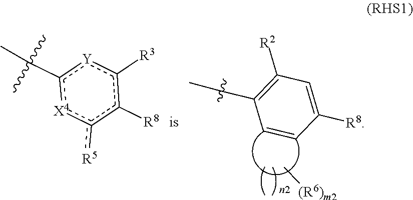

In certain embodiments of Formula V (when the moiety

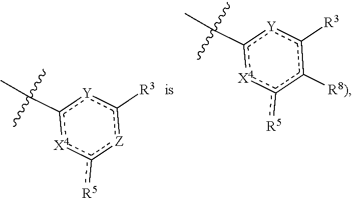

the moiety

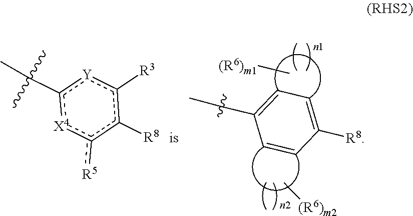

In certain embodiments of Formula V (when the moiety

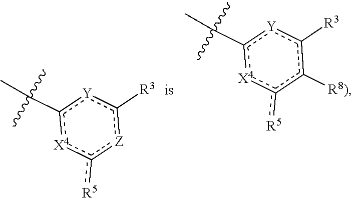

the moiety

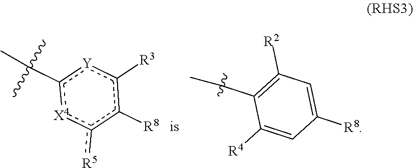

In certain embodiments of Formula V (when the moiety

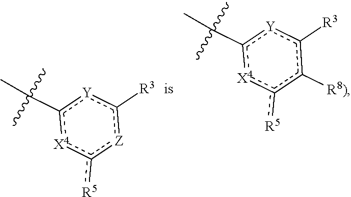

the moiety

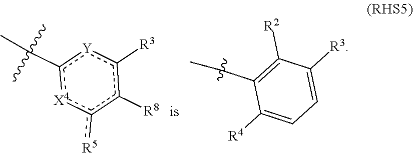

As a non-limiting example of above, RHS5 can be

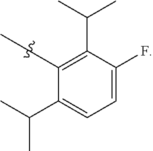 [SMILES: *c1c(C(C)C)ccc(F)c1C(C)C]

In certain embodiments of Formula V (when the moiety

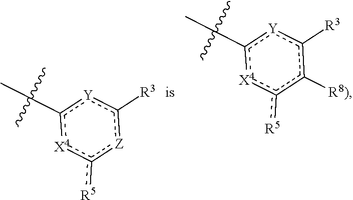

the moiety

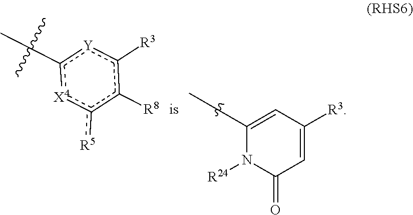

In certain embodiments of Formula V (when the moiety

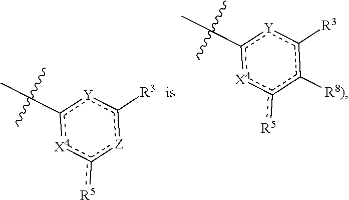

the moiety

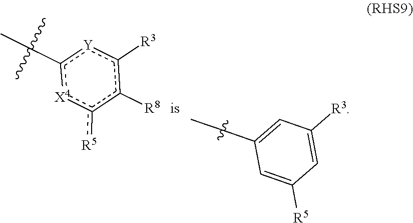

In certain embodiments of Formula V (when the moiety

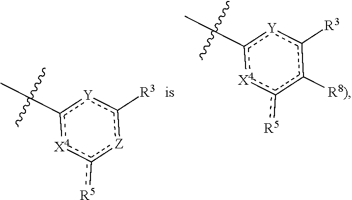

the moiety

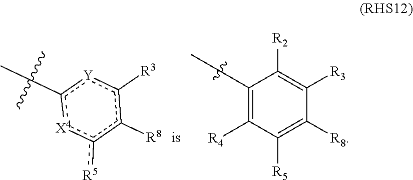

In some embodiments of the compound of Formula V, X¹⁰ is CR¹⁰.

In certain embodiments of the compound of Formula V, R¹⁰ is 2-hydroxy-2-propyl; R¹⁰ is 1-hydroxy-1-cyclopropyl; R¹⁰ is dimethylaminomethyl; or R¹⁰ is S(O₂)CH₃.

In some embodiments of the compound of Formula V, X¹¹ is CR¹.

In certain embodiments of the compound of Formula V, R¹ is 2-hydroxy-2-propyl; R¹ is 1-hydroxy-1-cyclopropyl; R¹ is dimethylaminomethyl; or R¹ is S(O₂)CH₃.

In some embodiments of the compound of Formula V, X¹⁰ is NR¹⁰.

In certain embodiments of the compound of Formula V, R¹⁰ is isopropyl; R¹⁰ is methyl; R¹⁰ is benzyl; or R¹⁰ is phenyl.

In some embodiments of the compound of Formula V, X³⁵ is CR³⁵.

In certain embodiments of the compound of Formula V, R³⁵ is 2-hydroxy-2-propyl; R³⁵ is 1-hydroxy-1-cyclopropyl; R³⁵ is dimethylaminomethyl; or R³⁵ is S(O₂)CH₃.

In some embodiments of the compound of Formula V, X²¹ is CR²¹.
In certain embodiments of the compound of Formula V, R²¹ is 2-hydroxy-2-propyl; R²¹ is 1-hydroxy-1-cyclopropyl; R²¹ is dimethylaminomethyl; R²¹ can be S(O₂)CH₃; R²¹ is halo; or R²¹ is CH₃.

In some embodiments of the compound of Formula V, R²⁹ is 2-hydroxy-2-propyl; R²⁹ is 1-hydroxy-1-cyclopropyl; R²⁹ is dimethylaminomethyl; R²⁹ is S(O₂)CH₃; R²⁹ is halo; or R²⁹ is CH₃.

In some embodiments of the compound of Formula V, X³⁶ is CR³⁶.

In certain embodiments of the compound of Formula V, R³⁶ is halo; or R³⁶ is CH₃.

In some embodiments of the compound of Formula V, R³⁴ is halo; or R³⁴ is CH₃.

In some embodiments of the compound of Formula V, each R²⁰ is hydrogen.

In some embodiments of the compound of Formula V

Ar is a heteroaryl group wherein

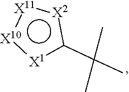

X¹ is O, S, N or CH;

X¹⁰ is CR¹⁰ or NR¹⁰;

X¹¹ is CR¹ or NR¹,

X² is O, S, N or CH;

each of R¹ and R¹⁰ when bonded to carbon is independently selected from H, C₁-C₆ alkyl, C₆-C₁₀ aryl, S(O₂)C₁-C₆ akyl and C₃-C₇ cycloalkyl, wherein the C₁-C₆ alkyl and C₃-C₇ cycloalkyl is

optionally substituted with one or more substituents each independently selected from hydroxy, oxo, C₁-C₆ alkoxy, and NR¹¹R¹²;

and each of R¹, R¹⁰ when bonded to nitrogen is independently selected from H, C₁-C₆ alkyl, C₆-C₁₀ aryl, and C₃-C₇ cycloalkyl, wherein the C₁-C₆ alkyl and C₃-C₇ cycloalkyl is optionally substituted with one or more substituents each independently selected from hydroxy and C₁-C₆ alkoxy;

R⁸ is selected from H, CN, Cl, F, C₁-C₆ alkyl, C₁-C₆ alkoxy, C₁-C₆ haloalkoxy, and C₁-C₆ haloalkyl;

R² is hydrogen, C₁-C₆ haloalkyl, C₃-C₇ cycloalkyl or C₁-C₆ alkyl;

R³ is hydrogen or halo;

R⁴ is hydrogen, C₁-C₆ haloalkyl, C₃-C₇ cycloalkyl or C₁-C₆ alkyl;

R⁵ is hydrogen or halo.

In some embodiments of the compound of Formula V, the compound of Formula V is a compound of Formula V-Ia

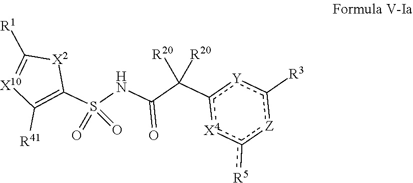

wherein

X¹⁰ is N or CR¹⁰;

and

X² is a, S, or NR⁴².

In some embodiments of the compound of Formula V-Ia, X¹⁰ is N; and X² is O.

In some embodiments of the compound of Formula V-Ia, X¹⁰ is N; and X² is S.

In some embodiments of the compound of Formula V-Ia, X¹⁰ is CR¹⁰; and X² is O.

In some embodiments of the compound of Formula V-Ia, X¹⁰ is CR¹⁰; and X² is S.

In some embodiments of the compound of Formula V-Ia, the compound of Formula

V is a compound of Formula V-Ib:

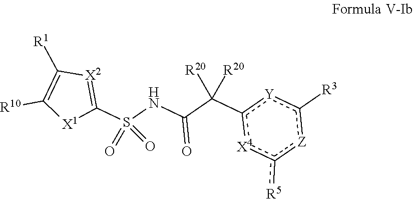

wherein

X¹ is O, S, or NR⁴¹;

and

X² is N or CR⁴².

In some embodiments of the compound of Formula V-Ib, X¹ is O; and X² is N.

In some embodiments of the compound of Formula V-Ib, X¹ is S; and X² is N.

In some embodiments of the compound of Formula V-Ib, X¹ is O; and X² is CR⁴².

In some embodiments of the compound of Formula V-Ib, X¹ is S; and X² is CR⁴².

In certain embodiments of the compound of Formula V-Ia or V-Ib, R¹ is 2-hydroxy-2-propyl; R¹ is 1-hydroxy-1-cyclopropyl; R¹ is dimethylaminomethyl; or R¹ is S(O₂)CH₃

In certain embodiments of the compound of Formula V-Ia or V-Ib, R¹⁰ is 2-hydroxy-2-propyl; R¹⁰ is 1-hydroxy-1-cyclopropyl; R¹⁰ is dimethylaminomethyl; or R¹⁰ is S(O₂)CH₃.

In certain embodiments of the compound of Formula V-Ia or V-Ib (when X¹ is NR⁴¹). R⁴¹ is 2-hydroxy-2-propyl; R⁴¹ is 1-hydroxy-1-cyclopropyl; R⁴¹ is dimethylaminomethyl; or R⁴¹ is S(O₂)CH₃.

In certain embodiments of the compound of Formula V-Ia or V-Ib, R⁴² is 2-hydroxy-2-propyl; R⁴² is 1-hydroxy-1-cyclopropyl; R⁴² is dimethylaminomethyl; or R⁴² is S(O₂)CH₃.

In some embodiments of the compound of Formula V,

Ar is an aryl or heteroaryl group

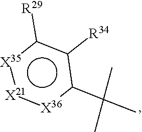

X³⁵ is CR³⁵;

X²¹ is N or CR²¹;

X³⁶ 1 S CR³⁶;

each of R³⁴, R²⁹, R³⁵, R²¹ and R³⁶ is independently selected from H, C₁-C₆ alkyl, halo, C₃-C₇ cycloalkyl, 3- to 7-membered nonaromatic monocyclic heterocycloalkyl, C₆-C₁₀ aryl, and S(O₂)C₁-C₆ alkyl;

wherein the C₁-C₆ alkyl, 3- to 7-membered nonaromatic monocyclic heterocycloalkyl, and C₃-C₇ cycloalkyl is optionally substituted with one or more substituents each independently selected from hydroxyl, C₁-C₆ alkyl, oxo, NR¹¹R¹², and 3- to 7-membered heterocycloalkyl,

R⁸ is selected from H, CN, Cl, F, C₁-C₆ alkyl, C₁-C₆ alkoxy, C₁-C₆ haloalkoxy, and C₁-C₆ haloalkyl;

R² is hydrogen, C₁-C₆ haloalkyl, C₃-C₇ cycloalkyl or C₁-C₆ alkyl; R³ is hydrogen or halo;

R⁴ is hydrogen, C₁-C₆ haloalkyl, C₃-C₇ cycloalkyl or C₁-C₆ alkyl;

R⁵ is hydrogen or halo.

In certain embodiments of the compound of Formula V, R³⁵ is 2-hydroxy-2-propyl; R³⁵ is 1-hydroxy-1-cyclopropyl; R³⁵ is dimethylaminomethyl; R³⁵ is S(O₂)CH₃; R³⁵ is methyl; or R³⁵ is halo.

In certain embodiments of the compound of Formula V (when X²¹ is R₂₁), R²¹ is 2-hydroxy-2-propyl; R²¹ is 1-hydroxy-1-cyclopropyl; R¹¹ is dimethylaminomethyl; R¹¹ is S(O₂)CH₃; R¹¹ is methy; or R¹¹ is halo.

In certain embodiments of the compound of Formula V, R²⁹ is 2-hydroxy-2-propyl; R²⁹ is 1-hydroxy-1-cyclopropyl; R²⁹ is dimethylaminomethyl; R²⁹ is S(O₂)CH₃; R²⁹ is methyl; or R²⁹ is halo.

In certain embodiments of the compound of Formula V, R³⁶ is methyl; or R³⁶ is halo.

In certain embodiments of the compound of Formula V, R³⁴ is methyl; or R³⁴ is halo.

In another aspect, an NLRP3 antagonist is a compound selected from the group consisting of the compounds below:

| | |
|---|---|
| 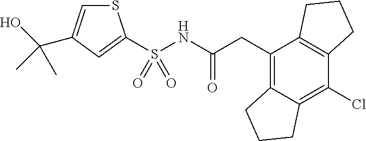 [SMILES: CC(C)(O)c1csc(S(=O)(=O)NC(=O)Cc2c3c(c(Cl)c4c2CCC4)CCC3)c1] | 101 |
| 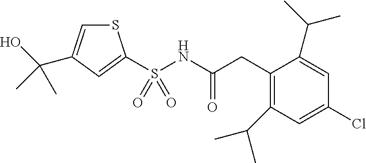 [SMILES: CC(C)c1cc(Cl)cc(C(C)C)c1CC(=O)NS(=O)(=O)c1cc(C(C)(C)O)cs1] | 102 |
| 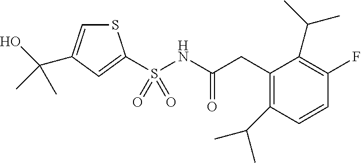 [SMILES: CC(C)c1ccc(F)c(C(C)C)c1CC(=O)NS(=O)(=O)c1cc(C(C)(C)O)cs1] | 103 |
| 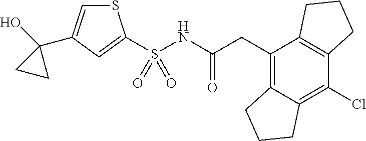 [SMILES: O=C(Cc1c2c(c(Cl)c3c1CCC3)CCC2)NS(=O)(=O)c1cc(C2(O)CC2)cs1] | 104 |
| 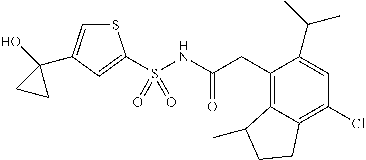 [SMILES: CC(C)c1cc(Cl)c2c(c1CC(=O)NS(=O)(=O)c1cc(C3(O)CC3)cs1)C(C)CC2] | 105 |
| 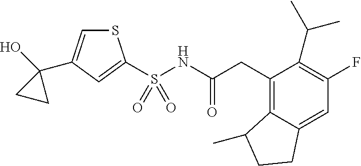 [SMILES: CC(C)c1c(F)cc2c(c1CC(=O)NS(=O)(=O)c1cc(C3(O)CC3)cs1)C(C)CC2] | 106 |
| 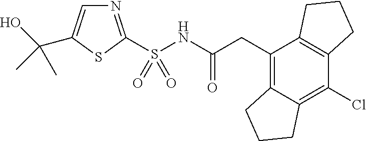 [SMILES: CC(C)(O)c1cnc(S(=O)(=O)NC(=O)Cc2c3c(c(Cl)c4c2CCC4)CCC3)s1] | 107 |
| 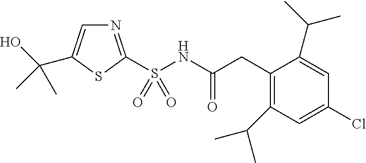 [SMILES: CC(C)c1cc(Cl)cc(C(C)C)c1CC(=O)NS(=O)(=O)c1ncc(C(C)(C)O)s1] | 108 |
| 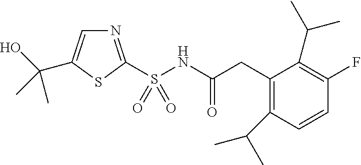 [SMILES: CC(C)c1ccc(F)c(C(C)C)c1CC(=O)NS(=O)(=O)c1ncc(C(C)(C)O)s1] | 109 |
| 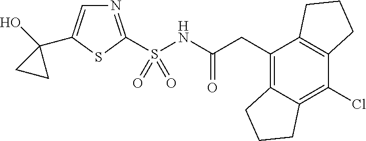 [SMILES: O=C(Cc1c2c(c(Cl)c3c1CCC3)CCC2)NS(=O)(=O)c1ncc(C2(O)CC2)s1] | 110 |
| 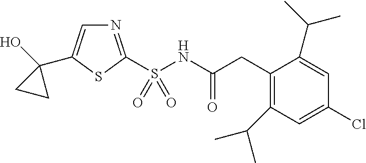 [SMILES: CC(C)c1cc(Cl)cc(C(C)C)c1CC(=O)NS(=O)(=O)c1ncc(C2(O)CC2)s1] | 111 |
| 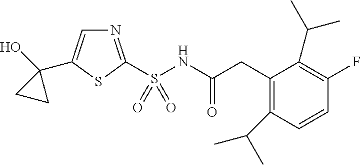 [SMILES: CC(C)c1ccc(F)c(C(C)C)c1CC(=O)NS(=O)(=O)c1ncc(C2(O)CC2)s1] | 112 |
| 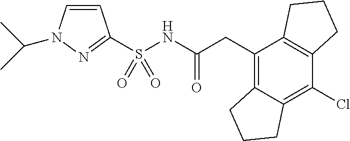 [SMILES: CC(C)n1ccc(S(=O)(=O)NC(=O)Cc2c3c(c(Cl)c4c2CCC4)CCC3)n1] | 113 |
| 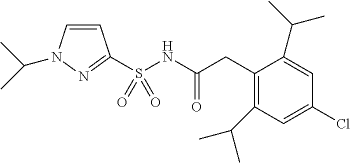 [SMILES: CC(C)c1cc(Cl)cc(C(C)C)c1CC(=O)NS(=O)(=O)c1ccn(C(C)C)n1] | 114 |
| 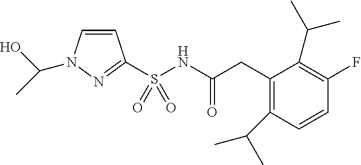 [SMILES: CC(C)c1ccc(F)c(C(C)C)c1CC(=O)NS(=O)(=O)c1ccn(C(C)O)n1] | 115 |
| 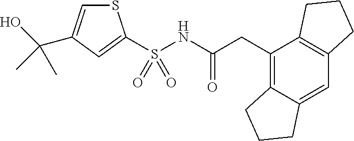 [SMILES: CC(C)(O)c1csc(S(=O)(=O)NC(=O)Cc2c3c(cc4c2CCC4)CCC3)c1] | 116 |
| 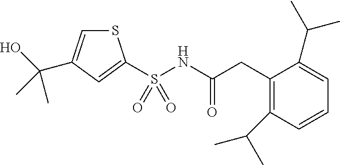 [SMILES: CC(C)c1cccc(C(C)C)c1CC(=O)NS(=O)(=O)c1cc(C(C)(C)O)cs1] | 117 |
| 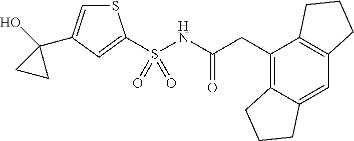 [SMILES: O=C(Cc1c2c(cc3c1CCC3)CCC2)NS(=O)(=O)c1cc(C2(O)CC2)cs1] | 118 |
| 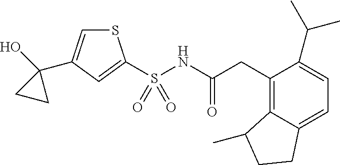 [SMILES: CC(C)c1ccc2c(c1CC(=O)NS(=O)(=O)c1cc(C3(O)CC3)cs1)C(C)CC2] | 119 |
| 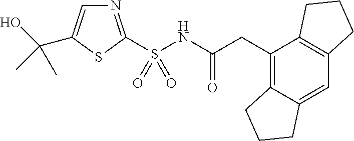 [SMILES: CC(C)(O)c1cnc(S(=O)(=O)NC(=O)Cc2c3c(cc4c2CCC4)CCC3)s1] | 120 |
| 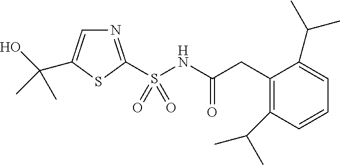 [SMILES: CC(C)c1cccc(C(C)C)c1CC(=O)NS(=O)(=O)c1ncc(C(C)(C)O)s1] | 121 |
| 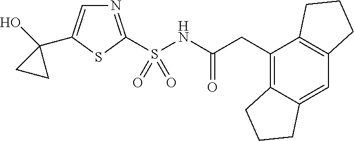 [SMILES: O=C(Cc1c2c(cc3c1CCC3)CCC2)NS(=O)(=O)c1ncc(C2(O)CC2)s1] | 122 |
| 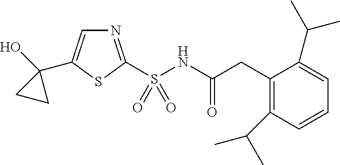 [SMILES: CC(C)c1cccc(C(C)C)c1CC(=O)NS(=O)(=O)c1ncc(C2(O)CC2)s1] | 123 |
| 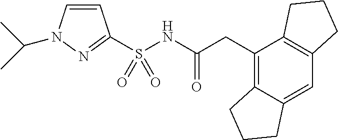 [SMILES: CC(C)n1ccc(S(=O)(=O)NC(=O)Cc2c3c(cc4c2CCC4)CCC3)n1] | 124 |
| 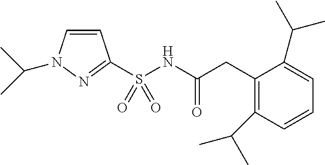 [SMILES: CC(C)c1cccc(C(C)C)c1CC(=O)NS(=O)(=O)c1ccn(C(C)C)n1] | 125 |
| 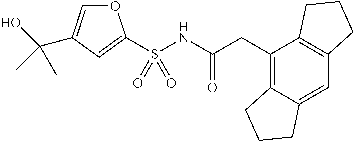 [SMILES: CC(C)(O)c1coc(S(=O)(=O)NC(=O)Cc2c3c(cc4c2CCC4)CCC3)c1] | 126 |

and pharmaceutically acceptable salts thereof.

In another aspect, an NLRP3 antagonist is compound selected from the group consisting of the compounds in Table 5 below and pharmaceutically acceptable salts thereof.

TABLE 5

| Com- | | | |
|---|---|---|---|
| pound | Structure | | |
| 127 | 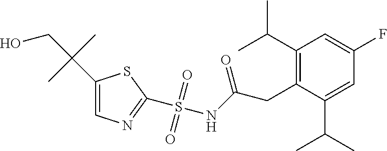 [SMILES: CC(C)c1cc(F)cc(C(C)C)c1CC(=O)NS(=O)(=O)c1ncc(C(C)(C)CO)s1] | 128 | 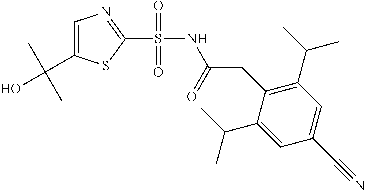 [SMILES: CC(C)c1cc(C#N)cc(C(C)C)c1CC(=O)NS(=O)(=O)c1ncc(C(C)(C)O)s1] |
| 129 | 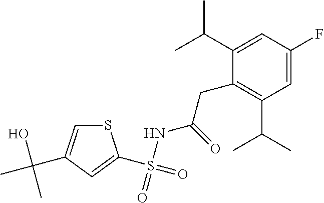 [SMILES: CC(C)c1cc(F)cc(C(C)C)c1CC(=O)NS(=O)(=O)c1cc(C(C)(C)O)cs1] | 130 | 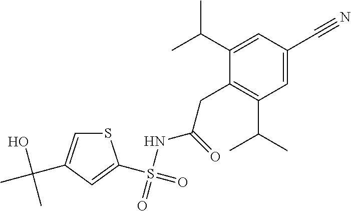 [SMILES: CC(C)c1cc(C#N)cc(C(C)C)c1CC(=O)NS(=O)(=O)c1cc(C(C)(C)O)cs1] |
| 131 | 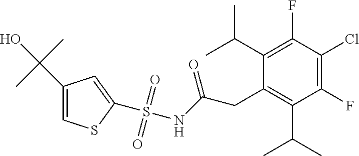 [SMILES: CC(C)c1c(F)c(Cl)c(F)c(C(C)C)c1CC(=O)NS(=O)(=O)c1cc(C(C)(C)O)cs1] | 132 | 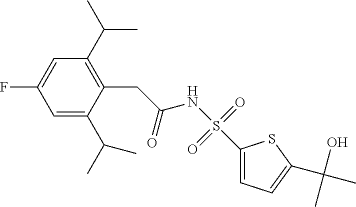 [SMILES: CC(C)c1cc(F)cc(C(C)C)c1CC(=O)NS(=O)(=O)c1ccc(C(C)(C)O)s1] |
| 133 | 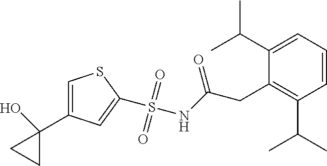 [SMILES: CC(C)c1cccc(C(C)C)c1CC(=O)NS(=O)(=O)c1cc(C2(O)CC2)cs1] | 134 | 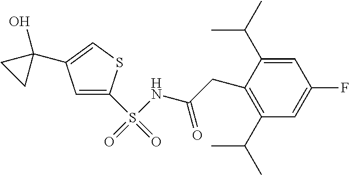 [SMILES: CC(C)c1cc(F)cc(C(C)C)c1CC(=O)NS(=O)(=O)c1cc(C2(O)CC2)cs1] |
| 135 | 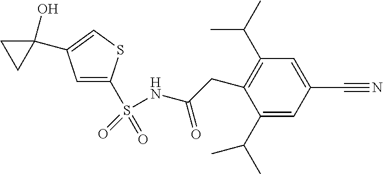 [SMILES: CC(C)c1cc(C#N)cc(C(C)C)c1CC(=O)NS(=O)(=O)c1cc(C2(O)CC2)cs1] | 136 | 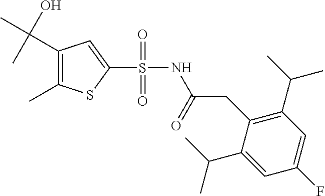 [SMILES: Cc1sc(S(=O)(=O)NC(=O)Cc2c(C(C)C)cc(F)cc2C(C)C)cc1C(C)(C)O] |
| 137 | 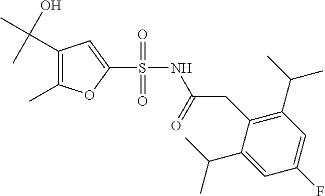 [SMILES: Cc1oc(S(=O)(=O)NC(=O)Cc2c(C(C)C)cc(F)cc2C(C)C)cc1C(C)(C)O] | 138 | 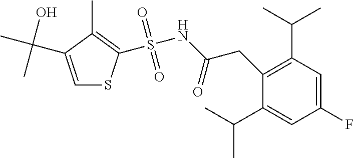 [SMILES: Cc1c(C(C)(C)O)csc1S(=O)(=O)NC(=O)Cc1c(C(C)C)cc(F)cc1C(C)C] |
| 139 | 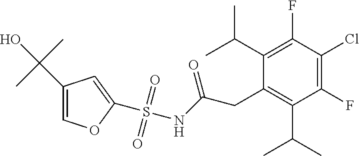 [SMILES: CC(C)c1c(F)c(Cl)c(F)c(C(C)C)c1CC(=O)NS(=O)(=O)c1cc(C(C)(C)O)co1] | | |
| 140 | 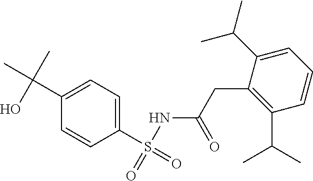 [SMILES: CC(C)c1cccc(C(C)C)c1CC(=O)NS(=O)(=O)c1ccc(C(C)(C)O)cc1] | 141 | 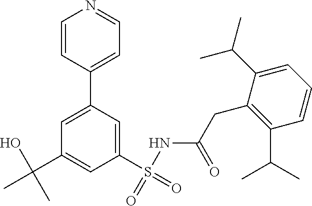 [SMILES: CC(C)c1cccc(C(C)C)c1CC(=O)NS(=O)(=O)c1cc(-c2ccncc2)cc(C(C)(C)O)c1] |
| 142 | 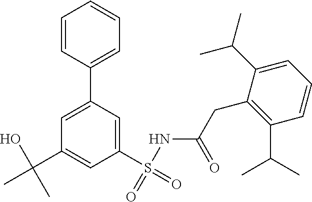 [SMILES: CC(C)c1cccc(C(C)C)c1CC(=O)NS(=O)(=O)c1cc(-c2ccccc2)cc(C(C)(C)O)c1] | 143 | 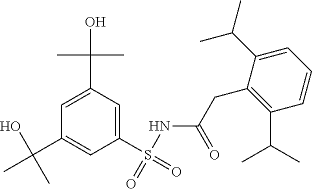 [SMILES: CC(C)c1cccc(C(C)C)c1CC(=O)NS(=O)(=O)c1cc(C(C)(C)O)cc(C(C)(C)O)c1] |
| 144 | 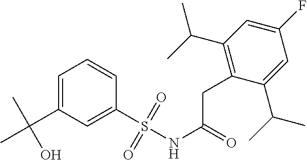 [SMILES: CC(C)c1cc(F)cc(C(C)C)c1CC(=O)NS(=O)(=O)c1cccc(C(C)(C)O)c1] | 145 | 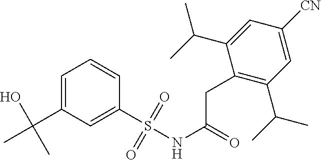 [SMILES: CC(C)c1cc(C#N)cc(C(C)C)c1CC(=O)NS(=O)(=O)c1cccc(C(C)(C)O)c1] |
| 146 | 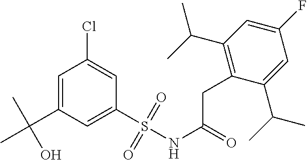 [SMILES: CC(C)c1cc(F)cc(C(C)C)c1CC(=O)NS(=O)(=O)c1cc(Cl)cc(C(C)(C)O)c1] | 147 | 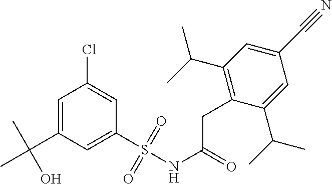 [SMILES: CC(C)c1cc(C#N)cc(C(C)C)c1CC(=O)NS(=O)(=O)c1cc(Cl)cc(C(C)(C)O)c1] |
| 148 | 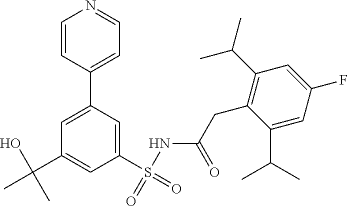 [SMILES: CC(C)c1cc(F)cc(C(C)C)c1CC(=O)NS(=O)(=O)c1cc(-c2ccncc2)cc(C(C)(C)O)c1] | 149 | 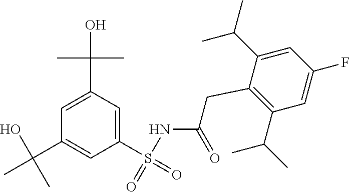 [SMILES: CC(C)c1cc(F)cc(C(C)C)c1CC(=O)NS(=O)(=O)c1cc(C(C)(C)O)cc(C(C)(C)O)c1] |
| 150 | 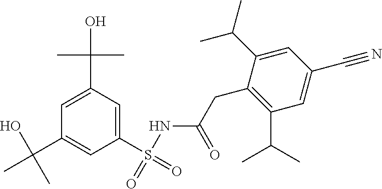 [SMILES: CC(C)c1cc(C#N)cc(C(C)C)c1CC(=O)NS(=O)(=O)c1cc(C(C)(C)O)cc(C(C)(C)O)c1] | 151 | 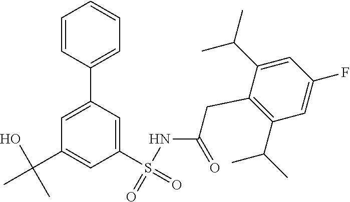 [SMILES: CC(C)c1cc(F)cc(C(C)C)c1CC(=O)NS(=O)(=O)c1cc(-c2ccccc2)cc(C(C)(C)O)c1] |
| 152 | 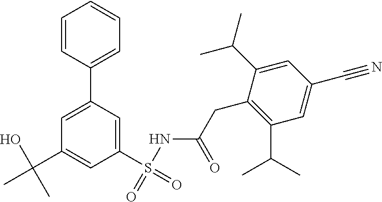 [SMILES: CC(C)c1cc(C#N)cc(C(C)C)c1CC(=O)NS(=O)(=O)c1cc(-c2ccccc2)cc(C(C)(C)O)c1] | 153 | 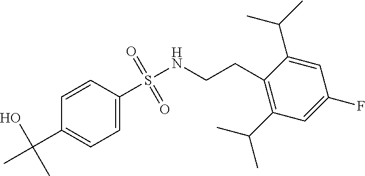 [SMILES: CC(C)c1cc(F)cc(C(C)C)c1CCNS(=O)(=O)c1ccc(C(C)(C)O)cc1] |
| 154 | 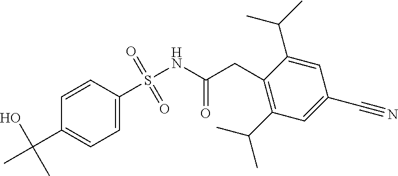 [SMILES: CC(C)c1cc(C#N)cc(C(C)C)c1CC(=O)NS(=O)(=O)c1ccc(C(C)(C)O)cc1] | 155 | 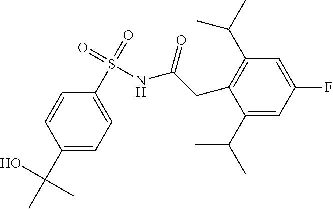 [SMILES: CC(C)c1cc(F)cc(C(C)C)c1CC(=O)NS(=O)(=O)c1ccc(C(C)(C)O)cc1] |
| 156 | 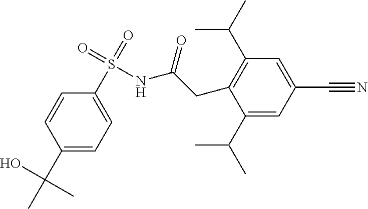 [SMILES: CC(C)c1cc(C#N)cc(C(C)C)c1CC(=O)NS(=O)(=O)c1ccc(C(C)(C)O)cc1] | 157 | 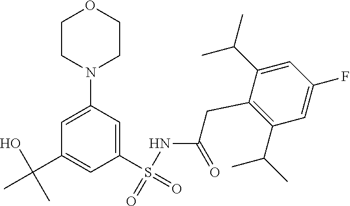 [SMILES: CC(C)c1cc(F)cc(C(C)C)c1CC(=O)NS(=O)(=O)c1cc(N2CCOCC2)cc(C(C)(C)O)c1] |
| 158 | 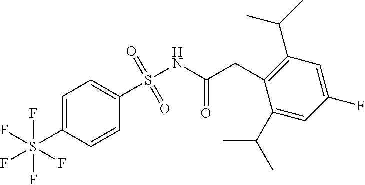 [SMILES: CC(C)c1cc(F)cc(C(C)C)c1CC(=O)NS(=O)(=O)c1ccc(S(F)(F)(F)(F)F)cc1] | 159 | 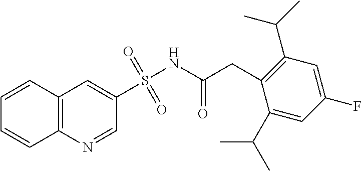 [SMILES: CC(C)c1cc(F)cc(C(C)C)c1CC(=O)NS(=O)(=O)c1cnc2ccccc2c1] |
| 160 | 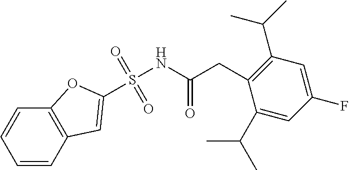 [SMILES: CC(C)c1cc(F)cc(C(C)C)c1CC(=O)NS(=O)(=O)c1cc2ccccc2o1] | 161 | 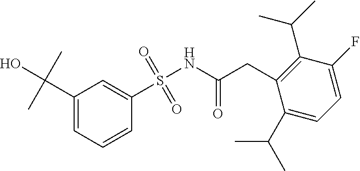 [SMILES: CC(C)c1ccc(F)c(C(C)C)c1CC(=O)NS(=O)(=O)c1cccc(C(C)(C)O)c1] |
| 162 | 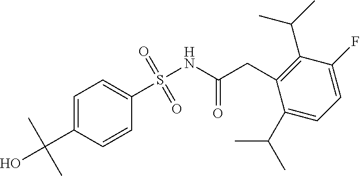 [SMILES: CC(C)c1ccc(F)c(C(C)C)c1CC(=O)NS(=O)(=O)c1ccc(C(C)(C)O)cc1] | 163 | 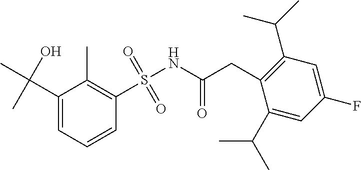 [SMILES: Cc1c(C(C)(C)O)cccc1S(=O)(=O)NC(=O)Cc1c(C(C)C)cc(F)cc1C(C)C] |
| 164 | 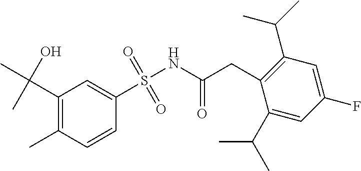 [SMILES: Cc1ccc(S(=O)(=O)NC(=O)Cc2c(C(C)C)cc(F)cc2C(C)C)cc1C(C)(C)O] | 165 | 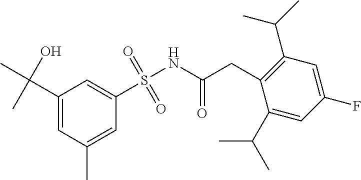 [SMILES: Cc1cc(C(C)(C)O)cc(S(=O)(=O)NC(=O)Cc2c(C(C)C)cc(F)cc2C(C)C)c1] |
| 166 | 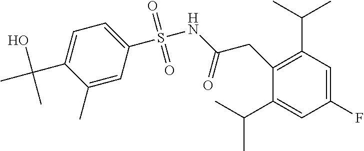 [SMILES: Cc1cc(S(=O)(=O)NC(=O)Cc2c(C(C)C)cc(F)cc2C(C)C)ccc1C(C)(C)O] | 167 | 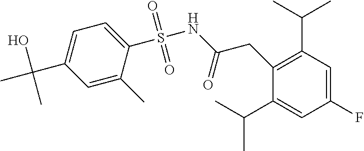 [SMILES: Cc1cc(C(C)(C)O)ccc1S(=O)(=O)NC(=O)Cc1c(C(C)C)cc(F)cc1C(C)C] |
| 168 | 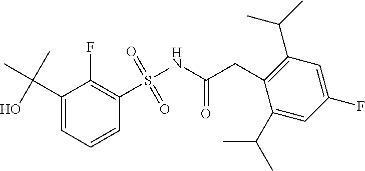 [SMILES: CC(C)c1cc(F)cc(C(C)C)c1CC(=O)NS(=O)(=O)c1cccc(C(C)(C)O)c1F] | 169 | 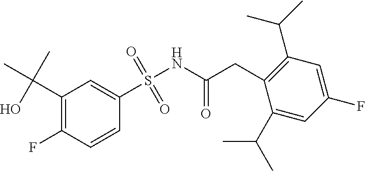 [SMILES: CC(C)c1cc(F)cc(C(C)C)c1CC(=O)NS(=O)(=O)c1ccc(F)c(C(C)(C)O)c1] |
| 170 | 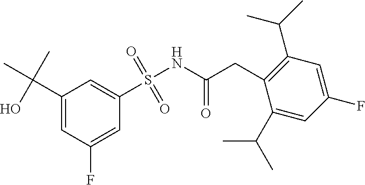 [SMILES: CC(C)c1cc(F)cc(C(C)C)c1CC(=O)NS(=O)(=O)c1cc(F)cc(C(C)(C)O)c1] | 171 | 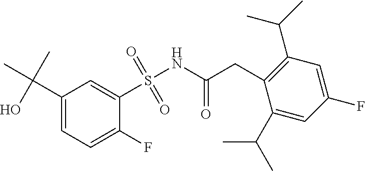 [SMILES: CC(C)c1cc(F)cc(C(C)C)c1CC(=O)NS(=O)(=O)c1cc(C(C)(C)O)ccc1F] |
| 172 | 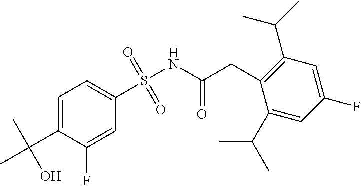 [SMILES: CC(C)c1cc(F)cc(C(C)C)c1CC(=O)NS(=O)(=O)c1ccc(C(C)(C)O)c(F)c1] | 173 | 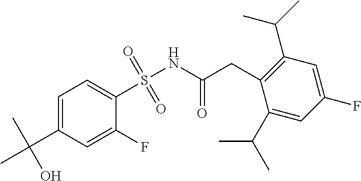 [SMILES: CC(C)c1cc(F)cc(C(C)C)c1CC(=O)NS(=O)(=O)c1ccc(C(C)(C)O)cc1F] |
| 174 | 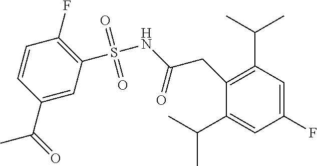 [SMILES: CC(=O)c1ccc(F)c(S(=O)(=O)NC(=O)Cc2c(C(C)C)cc(F)cc2C(C)C)c1] | 175 | 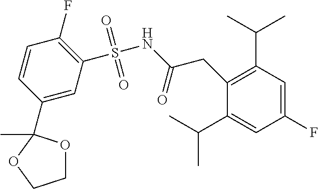 [SMILES: CC(C)c1cc(F)cc(C(C)C)c1CC(=O)NS(=O)(=O)c1cc(C2(C)OCCO2)ccc1F] |
| 176 | 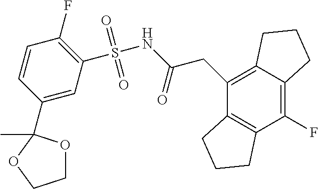 [SMILES: CC1(c2ccc(F)c(S(=O)(=O)NC(=O)Cc3c4c(c(F)c5c3CCC5)CCC4)c2)OCCO1] | 177 | 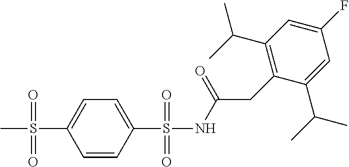 [SMILES: CC(C)c1cc(F)cc(C(C)C)c1CC(=O)NS(=O)(=O)c1ccc(S(C)(=O)=O)cc1] |
| 178 | 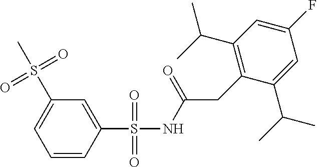 [SMILES: CC(C)c1cc(F)cc(C(C)C)c1CC(=O)NS(=O)(=O)c1cccc(S(C)(=O)=O)c1] | 179 | 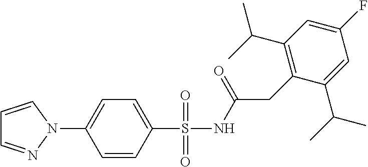 [SMILES: CC(C)c1cc(F)cc(C(C)C)c1CC(=O)NS(=O)(=O)c1ccc(-n2cccn2)cc1] |
| 180 | 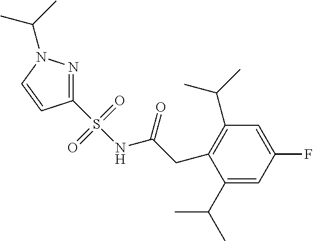 [SMILES: CC(C)c1cc(F)cc(C(C)C)c1CC(=O)NS(=O)(=O)c1ccn(C(C)C)n1] | 181 | 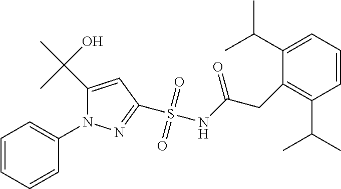 [SMILES: CC(C)c1cccc(C(C)C)c1CC(=O)NS(=O)(=O)c1cc(C(C)(C)O)n(-c2ccccc2)n1] |
| 182 | 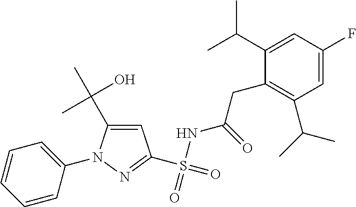 [SMILES: CC(C)c1cc(F)cc(C(C)C)c1CC(=O)NS(=O)(=O)c1cc(C(C)(C)O)n(-c2ccccc2)n1] | 183 | 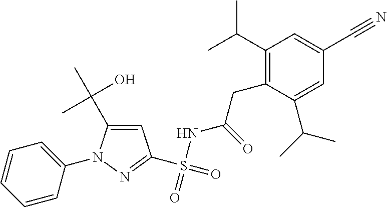 [SMILES: CC(C)c1cc(C#N)cc(C(C)C)c1CC(=O)NS(=O)(=O)c1cc(C(C)(C)O)n(-c2ccccc2)n1] |
| 184 | 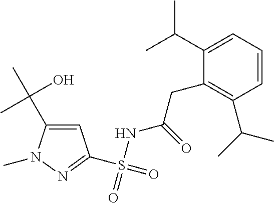 [SMILES: CC(C)c1cccc(C(C)C)c1CC(=O)NS(=O)(=O)c1cc(C(C)(C)O)n(C)n1] | 185 | 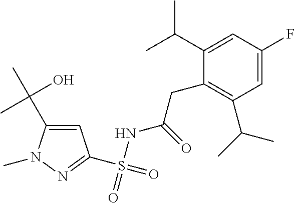 [SMILES: CC(C)c1cc(F)cc(C(C)C)c1CC(=O)NS(=O)(=O)c1cc(C(C)(C)O)n(C)n1] |
| 186 | 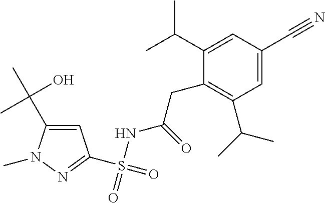 [SMILES: CC(C)c1cc(C#N)cc(C(C)C)c1CC(=O)NS(=O)(=O)c1cc(C(C)(C)O)n(C)n1] | 187 | 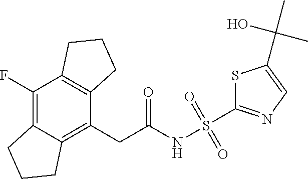 [SMILES: CC(C)(O)c1cnc(S(=O)(=O)NC(=O)Cc2c3c(c(F)c4c2CCC4)CCC3)s1] |
| 188 | 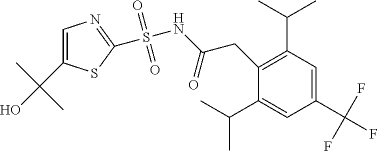 [SMILES: CC(C)c1cc(C(F)(F)F)cc(C(C)C)c1CC(=O)NS(=O)(=O)c1ncc(C(C)(C)O)s1] | 189 | 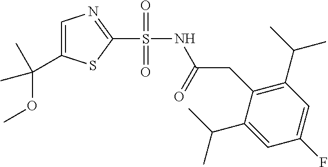 [SMILES: COC(C)(C)c1cnc(S(=O)(=O)NC(=O)Cc2c(C(C)C)cc(F)cc2C(C)C)s1] |
| 190 | 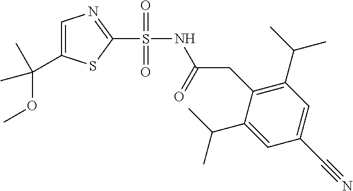 [SMILES: COC(C)(C)c1cnc(S(=O)(=O)NC(=O)Cc2c(C(C)C)cc(C#N)cc2C(C)C)s1] | 191 | 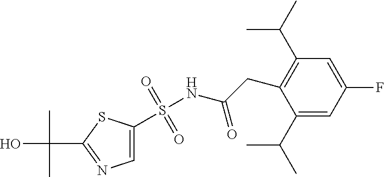 [SMILES: CC(C)c1cc(F)cc(C(C)C)c1CC(=O)NS(=O)(=O)c1cnc(C(C)(C)O)s1] |
| 192 | 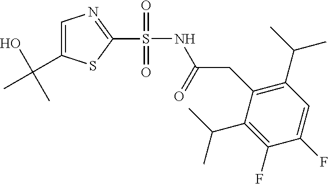 [SMILES: CC(C)c1cc(F)c(F)c(C(C)C)c1CC(=O)NS(=O)(=O)c1ncc(C(C)(C)O)s1] | 193 | 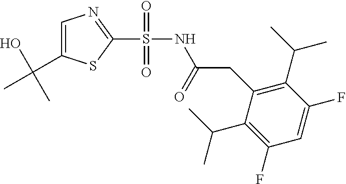 [SMILES: CC(C)c1c(F)cc(F)c(C(C)C)c1CC(=O)NS(=O)(=O)c1ncc(C(C)(C)O)s1] |
| 194 | 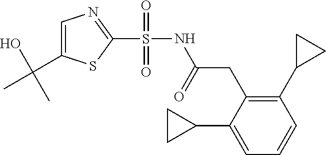 [SMILES: CC(C)(O)c1cnc(S(=O)(=O)NC(=O)Cc2c(C3CC3)cccc2C2CC2)s1] | 195 | 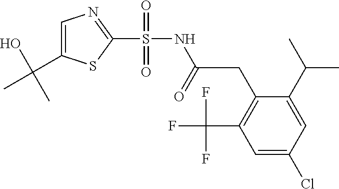 [SMILES: CC(C)c1cc(Cl)cc(C(F)(F)F)c1CC(=O)NS(=O)(=O)c1ncc(C(C)(C)O)s1] |
| 196 | 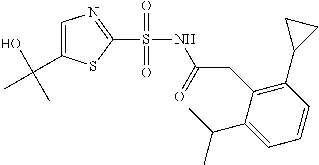 [SMILES: CC(C)c1cccc(C2CC2)c1CC(=O)NS(=O)(=O)c1ncc(C(C)(C)O)s1] | 197 | 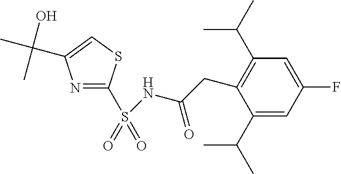 [SMILES: CC(C)c1cc(F)cc(C(C)C)c1CC(=O)NS(=O)(=O)c1nc(C(C)(C)O)cs1] |
| 198 | 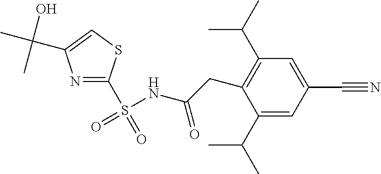 [SMILES: CC(C)c1cc(C#N)cc(C(C)C)c1CC(=O)NS(=O)(=O)c1nc(C(C)(C)O)cs1] | 199 | 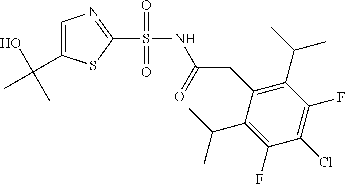 [SMILES: CC(C)c1c(F)c(Cl)c(F)c(C(C)C)c1CC(=O)NS(=O)(=O)c1ncc(C(C)(C)O)s1] |
| 200 | 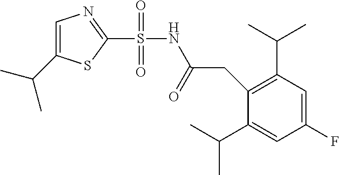 [SMILES: CC(C)c1cnc(S(=O)(=O)NC(=O)Cc2c(C(C)C)cc(F)cc2C(C)C)s1] | 201 | 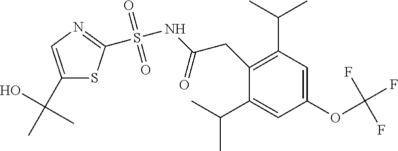 [SMILES: CC(C)c1cc(OC(F)(F)F)cc(C(C)C)c1CC(=O)NS(=O)(=O)c1ncc(C(C)(C)O)s1] |
| 202 | 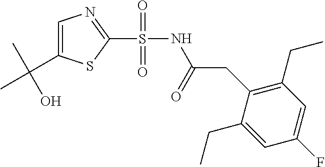 [SMILES: CCc1cc(F)cc(CC)c1CC(=O)NS(=O)(=O)c1ncc(C(C)(C)O)s1] | 203 | 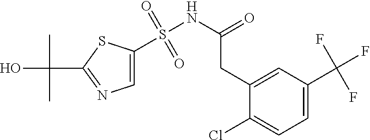 [SMILES: CC(C)(O)c1ncc(S(=O)(=O)NC(=O)Cc2cc(C(F)(F)F)ccc2Cl)s1] |
| 204 | 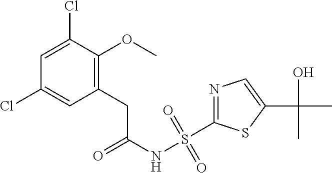 [SMILES: COc1c(Cl)cc(Cl)cc1CC(=O)NS(=O)(=O)c1ncc(C(C)(C)O)s1] | 205 | 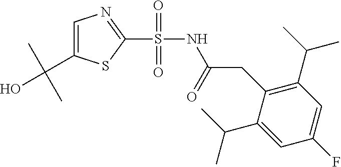 [SMILES: CC(C)c1cc(F)cc(C(C)C)c1CC(=O)NS(=O)(=O)c1ncc(C(C)(C)O)s1] |
| 206 | 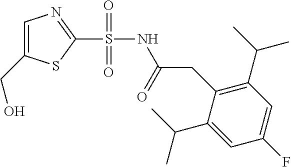 [SMILES: CC(C)c1cc(F)cc(C(C)C)c1CC(=O)NS(=O)(=O)c1ncc(CO)s1] | 207 | 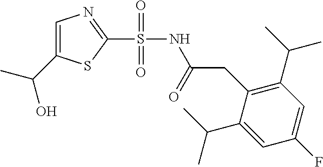 [SMILES: CC(C)c1cc(F)cc(C(C)C)c1CC(=O)NS(=O)(=O)c1ncc(C(C)O)s1] |
| 208 | 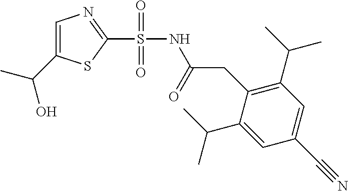 [SMILES: CC(C)c1cc(C#N)cc(C(C)C)c1CC(=O)NS(=O)(=O)c1ncc(C(C)O)s1] | 209 | 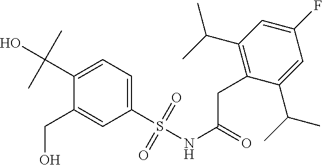 [SMILES: CC(C)c1cc(F)cc(C(C)C)c1CC(=O)NS(=O)(=O)c1ccc(C(C)(C)O)c(CO)c1] |
| 210 | 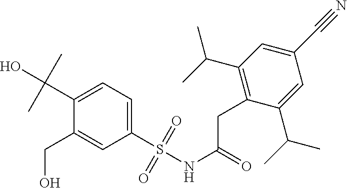 [SMILES: CC(C)c1cc(C#N)cc(C(C)C)c1CC(=O)NS(=O)(=O)c1ccc(C(C)(C)O)c(CO)c1] | 211 | 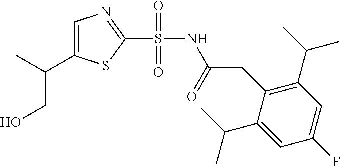 [SMILES: CC(C)c1cc(F)cc(C(C)C)c1CC(=O)NS(=O)(=O)c1ncc(C(C)CO)s1] |
| 212 | 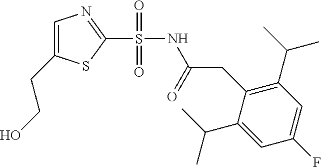 [SMILES: CC(C)c1cc(F)cc(C(C)C)c1CC(=O)NS(=O)(=O)c1ncc(CCO)s1] | 213 | 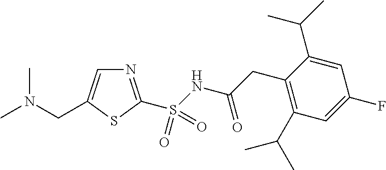 [SMILES: CC(C)c1cc(F)cc(C(C)C)c1CC(=O)NS(=O)(=O)c1ncc(CN(C)C)s1] |
| 214 | 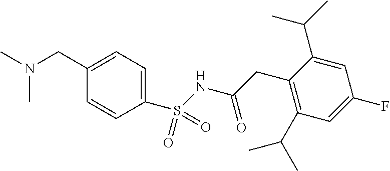 [SMILES: CC(C)c1cc(F)cc(C(C)C)c1CC(=O)NS(=O)(=O)c1ccc(CN(C)C)cc1] | 215 | 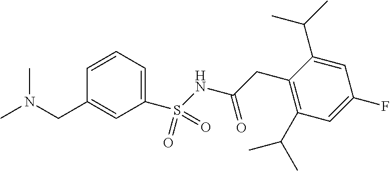 [SMILES: CC(C)c1cc(F)cc(C(C)C)c1CC(=O)NS(=O)(=O)c1cccc(CN(C)C)c1] |

Compounds having Formula V, as well as methods of making and using the same, are further described in WO2017184604A1 (PCT/US2017/028139), filed on Apr. 18, 2017; U.S. Provisional 62/324,071, filed on Apr. 18, 2016; U.S. Provisional 62/324,081, filed on Apr. 18, 2016; and U.S. Provisional 62/411,358, filed on Oct. 21, 2016, each of which is incorporated herein by reference in its entirety.

In one aspect, an NLRP3 antagonist is a compound of Formula VI

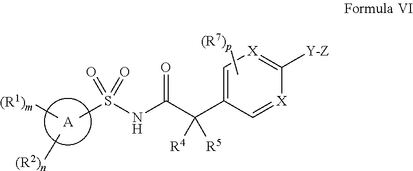

or a pharmaceutically acceptable salt thereof,

wherein

m=0, 1, or 2;

n=0, 1, or 2;

p=0, 1, or 2;

wherein

A is a 5-10-membered monocyclic or bicyclic heteroaryl, a 5-10-membered monocyclic or bicyclic heterocycloalkyl, a C₆-C₁₀ monocyclic or bicyclic aryl, or a C₆-C₁₀ monocyclic or bicyclic cycloalkyl;

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², SO₂NR⁸R⁹, NR¹¹SO₂NR¹¹R¹²NR¹¹CO₂R¹², NR¹¹CONR¹¹R¹², NR¹¹SO_{2R}¹², C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), and NHCOC₂-C₆ alkynyl;

wherein each C₁-C₆ alkyl substituent and each C₁-C₆ alkoxy substituent of the R¹ or R² C₃-C₇ cycloalkyl or of the R¹ or R² 3-to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;

or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R^{9′} wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;

Y is selected from a bond, O, S, SO₂, NR¹⁵, or CR¹⁶R¹⁷;

Z is selected from a 5-10-membered monocyclic or bicyclic heteroaryl, a 5-10-membered monocyclic or bicyclic heterocycloalkyl, a C₆-C₁₀ monocyclic or bicyclic aryl, a C₆-C₁₀ monocyclic or bicyclic cycloalkyl, or a C₂-C₆ alkenyl, wherein Z is optionally substituted with one or more substituents independently selected from C₁-C₆ alkyl, C₁-C₆ alkoxy, C₆-C₁₀ aryloxy, hydroxy, CN, halo, NR⁸R⁹, COOC₁-C₆ alkyl, S(O₂)C₁-C₆ alkyl, SO₂NR⁸R⁹, NR¹¹SO₂NR¹¹R¹², NR¹¹CO₂R¹², NR¹¹CONR¹¹R¹², NR¹¹SO₂R¹², 3- to 7-membered heterocycloalkyl, C₆-C₁₀ aryl, and CONR⁸R⁹, and wherein the C₁-C₆ alkyl or C₁-C₆ alkoxy that Z is substituted with is optionally substituted with one or more hydroxyl, NR⁸R⁹, or C₆-C₁₀ aryl, or wherein Z is optionally fused to a five-to-seven-membered carbocyclic ring or heterocyclic ring containing one or two heteroatoms independently selected from oxygen, sulfur and nitrogen;

each R⁷ is independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, OH, NHC₁-C₆ alkyl, N(C₁-C₆ alkyl)₂, CONR⁸R⁹, SO₂NR⁸R⁹, NR¹¹SO₂NR¹¹R¹², NR¹¹CO₂R¹², NR¹¹CONR¹¹R¹², NR¹¹SP₂R¹², SF₅, S(O₂)C₁-C₆ alkyl, C₃-C₇ cycloalkyl, and 3- to 7-membered heterocycloalkyl,

wherein the C₁-C₆ alkyl, C₁-C₆ haloalkyl, C₁-C₆ alkoxy, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), and NHCOC₂-C₆ alkynyl;

wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;

each X is independently N or CR⁶;

each R⁶ is independently selected from hydrogen, C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, OH, NHC₁-C₆ alkyl, N(C₁-C₆ alkyl)₂, CONR⁸R⁹, SP₂NR⁸R⁹, NR¹¹SP₂NR¹¹R¹², NR¹¹CO₂R¹², NR¹¹CONR¹¹R¹², NR¹¹SP₂R¹², SF₅, S(O₂)C₁-C₆ alkyl, C₃-C₇ cycloalkyl, and 3- to 7-membered heterocycloalkyl,

wherein the C₁-C₆ alkyl, C₁-C₆ haloalkyl, C₁-C₆ alkoxy, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

each of R⁴ and R⁵ is independently selected from hydrogen, C₁-C₆ alkyl, C₁-C₆ alkoxy, NH₂, NHC₁-C₆ alkyl, or N(C₁-C₆ alkyl)₂.

R¹⁰ is C₁-C₆ alkyl;

each of R⁸ and R⁹ at each occurrence is independently selected from hydrogen, C₁-C₆ alkyl, NH—(C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;

R¹³ is C₁-C₆ alkyl, C₆-C₁₀ aryl, or 5- to 10-membered heteroaryl;

each of R¹¹ and R¹² at each occurrence is independently selected from hydrogen and C₁-C₆ alkyl;

R¹⁵ is selected from H or C₁-C₆ alkyl;

R¹⁶ is selected from H or C₁-C₆ alkyl; and

R¹⁷ is selected from H or C₁-C₆ alkyl.

In another aspect, an NLRP3 antagonist is a compound of Formula VI, wherein the compound is a compound of Formula VI-I

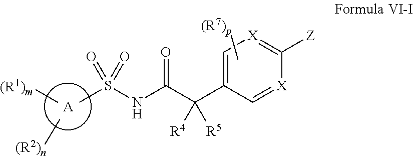

wherein

m=0, 1, or 2;

n=0, 1, or 2;

p=0, 1, or 2;

wherein

A is a 5-10-membered monocyclic or bicyclic heteroaryl, a 5-10-membered monocyclic or bicyclic heterocycloalkyl, a C₆-C₁₀ monocyclic or bicyclic aryl, or a C₆-C₁₀ monocyclic or bicyclic cycloalkyl;

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, NHCOOCC₁-C₆ alkyl, NH—(C═NR¹³)NR¹¹R¹², CONR⁸R⁹, SO₂NR⁸R⁹, NR¹¹SP₂NR¹¹R¹², NR¹¹CO₂R¹², NR¹¹CONR¹¹R¹², NR¹¹SO₂R¹², SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3-to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;

or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;

Z is selected from a 5-10-membered monocyclic or bicyclic heteroaryl, a 5-10-membered monocyclic or bicyclic heterocycloalkyl, a C₆-C₁₀ monocyclic or bicyclic aryl, a C₆-C₁₀ monocyclic or bicyclic cycloalkyl, or a C₂-C₆ alkenyl, wherein Z is optionally substituted with one or more substituents independently selected from C₁-C₆ alkyl, C₁-C₆ alkoxy, C₆-C₁₀ aryloxy, hydroxy, CN, halo, NR⁸R⁹, COOC₁-C₆ alkyl, S(O₂)C₁-C₆ alkyl, SO₂NR⁸R⁹, NR¹¹SP₂NR¹¹R¹², NR¹¹CO₂R¹², NR¹¹CONR¹¹R¹², NR¹¹SO₂R¹², 3- to 7-membered heterocycloalkyl, C₆-C₁₀ aryl, and CONR⁸R⁹, and wherein the C₁-C₆ alkyl or C₁-C₆ alkoxy that Z is substituted with is optionally substituted with one or more hydroxyl, NR⁸R⁹, or C₆-C₁₀ aryl;

or wherein Z is optionally fused to a five-to-seven-membered carbocyclic ring or heterocyclic ring containing one or two heteroatoms independently selected from oxygen, sulfur and nitrogen;

each R⁷ is independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, OH, NHC₁-C₆ alkyl, N(C₁-C₆ alkyl)₂, CONR⁸R⁹, SO₂NR⁸R⁹, NR¹¹SP₂NR¹¹R¹², NR¹¹CO₂R¹², NR¹¹CONR¹¹R¹², NR¹¹SP₂R¹², SF₅, S(O₂)C₁-C₆ alkyl, C₃-C₇ cycloalkyl, and 3- to 7-membered heterocycloalkyl,

wherein the C₁-C₆ alkyl, C₁-C₆ haloalkyl, C₁-C₆ alkoxy, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;

each X is independently N or CR⁶;

each R⁶ is independently selected from hydrogen, C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, OH, NHC₁-C₆ alkyl, N(C₁-C₆ alkyl)₂, CONR⁸R⁹, SO₂NR⁸R⁹, NR¹¹SP₂NR¹¹R¹², NR¹¹CO₂R¹², NR¹¹CONR¹¹R¹², NR¹¹SO_{2R}¹², SF₅, S(O₂)C₁-C₆ alkyl, C₃-C₇ cycloalkyl, and 3- to 7-membered heterocycloalkyl,

wherein the C₁-C₆ alkyl, C₁-C₆ haloalkyl, C₁-C₆ alkoxy, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

each of R⁴ and R⁵ is independently selected from hydrogen, C₁-C₆ alkyl, C₁-C₆ alkoxy, NH₂, NHC₁-C₆ alkyl, or N(C₁-C₆ alkyl)₂.

R₁₀ is C₁-C₆ alkyl;

each of R⁸ and R⁹ at each occurrence is independently selected from hydrogen, C₁-C₆ alkyl, NH—(C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;

R¹³ is C₁-C₆ alkyl, C₆-C₁₀ aryl, or 5- to 10-membered heteroaryl; and

each of R¹¹ and R¹² at each occurrence is independently selected from hydrogen and C₁-C₆ alkyl.

In some embodiments of the compound of Formula VI-I,

m=0, 1, or 2;

n=0, 1, or 2;

p=0, 1, or 2;

wherein

A is a 5-10-membered monocyclic or bicyclic heteroaryl, a 5-10-membered monocyclic or bicyclic heterocycloalkyl, a C₆-C₁₀ monocyclic or bicyclic aryl, or a C₆-C₁₀ monocyclic or bicyclic cycloalkyl;

R¹ and R² are each independently selected from C₁-C₆ alkyl, C₁-C₆ alkoxy, halo, CN, NO₂, COC₁-C₆ alkyl, CO₂C₁-C₆ alkyl, NH₂, NHC₁-C₆ alkyl, N(C₁-C₆ alkyl)₂, NHCOC₁-C₆ alkyl, CONR⁸R⁹, S(O₂)C₁-C₆ alkyl, S(O)C₁-C₆ alkyl, S(O₂)NR¹¹R¹², C₃-C₇ cycloalkyl and 3- to 7-membered heterocycloalkyl,

wherein the C₁-C₆ alkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, and NR⁸R⁹,

Z is selected from a 5-10-membered monocyclic or bicyclic heteroaryl, a 5-10-membered monocyclic or bicyclic heterocycloalkyl, a C₆-C₁₀ monocyclic or bicyclic aryl, a C₆-C₁₀ monocyclic or bicyclic cycloalkyl, or a C₂-C₆ alkenyl, wherein Z is optionally substituted with one or more substituents independently selected from C₁-C₆ alkyl, C₁-C₆ alkoxy, C₆-C₁₀ aryloxy, CN, halo, COOC₁-C₆ alkyl, S(O₂)C₁-C₆ alkyl, 3- to 7-membered heterocycloalkyl, and CONR⁸R⁹, and wherein the C₁-C₆ alkyl or C₁-C₆ alkoxy that Z is substituted with is optionally substituted with one or more hydroxyl, NR⁸R⁹, or C₆-C₁₀ aryl;

or wherein Z is optionally fused to a five-to-seven-membered carbocyclic ring or heterocyclic ring containing one or two heteroatoms independently selected from oxygen, sulfur and nitrogen;

each R⁷ is independently selected from C₁-C₆ alkyl, C₁-C₆ haloalkyl, C₁-C₆ alkoxy, halo, CN, COC₁-C₆ alkyl, CO₂C₃-C₈ cycloalkyl, NH₂, OH, S(O₂)C₁-C₆ alkyl, and C₃-C₇ cycloalkyl,

wherein the C₁-C₆ alkyl and C₁-C₆ alkoxy is optionally substituted with one or more substituents each independently selected from hydroxy, halo, CN, oxo, C₁-C₆ alkyl, C₁-C₆ alkoxy, NR⁸R⁹, and C₆-C₁₀ aryl,

each X is independently N or CR⁶;

each R⁶ is independently selected from hydrogen, C₁-C₆ alkyl, C₁-C₆ haloalkyl, C₁-C₆ alkoxy, halo, CN, COC₁-C₆ alkyl, CO₂C₃-C₈ cycloalkyl, NH₂, OH, S(O₂)C₁-C₆ alkyl, and C₃-C₇ cycloalkyl,

wherein the C₁-C₆ alkyl and C₁-C₆ alkoxy is optionally substituted with one or more substituents each independently selected from hydroxy, halo, CN, oxo, C₁-C₆ alkyl, C₁-C₆ alkoxy, NR⁸R⁹, C₆-C₁₀ aryl;

each of R⁴ and R⁵ is independently selected from hydrogen, C₁-C₆ alkyl, C₁-C₆ alkoxy, NH₂, NHC₁-C₆ alkyl, or N(C₁-C₆ alkyl)₂.

each of R⁸ and R⁹ at each occurrence is independently selected from hydrogen, C₁-C₆ alkyl, NH—(C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;

R¹³ is C₁-C₆ alkyl, C₆-C₁₀ aryl, or 5- to 10-membered heteroaryl; and each of R¹¹ and R¹² at each occurrence is independently selected from hydrogen and C₁-C₆ alkyl.

In some embodiments of the compound of Formula VI-I,

m=0 or 1;

n=0 or 1;

p=0, 1, or 2;

wherein

A is a 5-10-membered monocyclic or bicyclic heteroaryl, a 5-10-membered monocyclic or bicyclic heterocycloalkyl, a C₆-C₁₀ monocyclic or bicyclic aryl, or a C₆-C₁₀ monocyclic or bicyclic cycloalkyl;

R¹ and R² are each independently selected from C₁-C₆ alkyl, C₁-C₆ alkoxy, halo, COC₁-C₆ alkyl, CO₂C₁-C₆ alkyl, NH₂, NHC₁-C₆ alkyl, CONR⁸R⁹, S(O₂)C₁-C₆ alkyl, S(O₂)NR¹¹R¹², C₃-C₇ cycloalkyl and 3- to 7-membered heterocycloalkyl,

wherein the C₁-C₆ alkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, and NR⁸R⁹,

Z is selected from a 5-10-membered monocyclic or bicyclic heteroaryl, a 5-10-membered monocyclic or bicyclic heterocycloalkyl, a C₆-C₁₀ monocyclic or bicyclic aryl, a C₆-C₁₀ monocyclic or bicyclic cycloalkyl, or a C₂-C₆ alkenyl, wherein Z is optionally substituted with one or more substituents independently selected from C₁-C₆ alkyl, C₁-C₆ alkoxy, C₆-C₁₀ aryloxy, CN, halo, COOC₁-C₆ alkyl, S(O₂)C₁-C₆ alkyl, 3- to 7-membered heterocycloalkyl, and CONR⁸R⁹, and wherein the C₁-C₆ alkyl or C₁-C₆ alkoxy that Z is substituted with is optionally substituted with one or more hydroxyl, NR⁸R⁹, or C₆-C₁₀ aryl;

or wherein Z is optionally fused to a five-to-seven-membered carbocyclic ring or heterocyclic ring containing one or two heteroatoms independently selected from oxygen, sulfur and nitrogen;

each R⁷ is independently selected from C₁-C₆ alkyl, C₁-C₆ haloalkyl, C₁-C₆ alkoxy, halo, CN, COC₁-C₆ alkyl, CO₂C₃-C₈ cycloalkyl, NH₂, OH, S(O₂)C₁-C₆ alkyl, and C₃-C₇ cycloalkyl,

wherein the C₁-C₆ alkoxy is optionally substituted with one or more C₆-C₁₀ aryl,

each X is independently N or CR⁶;

each R⁶ is independently selected from hydrogen, C₁-C₆ alkyl, C₁-C₆ haloalkyl, C₁-C₆ alkoxy, halo, CN, COC₁-C₆ alkyl, CO₂C₃-C₈ cycloalkyl, NH₂, OH, S(O₂)C₁-C₆ alkyl, and C₃-C₇ cycloalkyl,

wherein the C₁-C₆ alkoxy is optionally substituted with one or more C₆-C₁₀ aryl;

each of R⁴ and R⁵ is independently selected from hydrogen, C₁-C₆ alkyl, C₁-C₆ alkoxy, NH₂, NHC₁-C₆ alkyl, or N(C₁-C₆ alkyl)₂.

each of R⁸ and R⁹ at each occurrence is independently selected from hydrogen and C₁-C₆ alkyl.

In some embodiments of the compound of Formula VI-I,

m=1;

n=0;

p=0 or 2;

wherein

A is a phenyl;

R¹ is (dimethylamino)methyl;

Z is selected from a 5-10-membered monocyclic or bicyclic heteroaryl, a 5-10-membered monocyclic or bicyclic heterocycloalkyl, a C₆-C₁₀ monocyclic or bicyclic aryl, a C₆-C₁₀ monocyclic or bicyclic cycloalkyl, or a C₂-C₆ alkenyl, wherein Z is optionally substituted with one or more substituents independently selected from C₁-C₆ alkyl, C₁-C₆ alkoxy, C₆-C₁₀ aryloxy, CN, halo, COOC₁-C₆ alkyl, S(O₂)C₁-C₆ alkyl, 3- to 7-membered heterocycloalkyl, and CONR⁸R⁹, and wherein the C₁-C₆ alkyl or C₁-C₆ alkoxy that Z is substituted with is optionally substituted with one or more hydroxyl, NR⁸R⁹, or C₆-C₁₀ aryl;

or wherein Z is optionally fused to a five-to-seven-membered carbocyclic ring or heterocyclic ring containing one or two heteroatoms independently selected from oxygen, sulfur and nitrogen;

each R⁷ is independently selected from C₁-C₆ alkyl and C₁-C₆ alkoxy,

wherein the C₁-C₆ alkoxy is optionally substituted with one or more C₆-C₁₀ aryl,

each X is CR⁶;

each R⁶ is independently selected from hydrogen, C₁-C₆ alkyl and C₁-C₆ alkoxy,

wherein the C₁-C₆ alkoxy is optionally substituted with one or more C₆-C₁₀ aryl; and

each of R⁴ and R⁵ is hydrogen

each of R⁸ and R⁹ at each occurrence is independently selected from hydrogen and C₁-C₆ alkyl.

In some embodiments of the compound of Formula VI or VI-I, A is thiazolyl optionally substituted with 1 or 2 R¹ and optionally substituted with 1 or 2 R².

In some embodiments of the compound of Formula VI or VI-I, A is phenyl optionally substituted with 1 or 2 R¹ and optionally substituted with 1 or 2 R².

In some embodiments of the compound of Formula VI or VI-I, m=1 and n=0.

In some embodiments of the compound of Formula VI or VI-I, A is

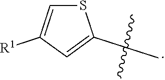

In some embodiments of the compound of Formula VI or VI-I, A is

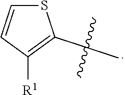

In some embodiments of the compound of Formula VI or VI-I, A is

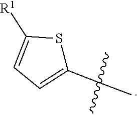

In some embodiments of the compound of Formula VI or VI-I, A is

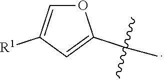

In some embodiments of the compound of Formula VI or VI-I, A is

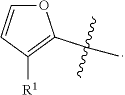

In some embodiments of the compound of Formula VI or VI-I, A is

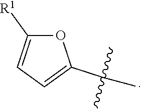

In some embodiments of the compound of Formula VI or VI-I, A is

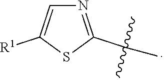

In some embodiments of the compound of Formula VI or VI-I, A is

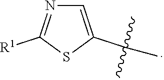

In some embodiments of the compound of Formula VI or VI-I, A is

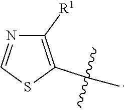

In some embodiments of the compound of Formula VI, A is

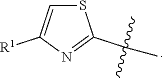

In some embodiments of the compound of Formula VI or VI-I, A is

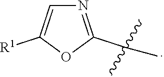

In some embodiments of the compound of Formula VI or VI-I, A is

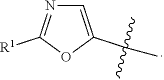

In some embodiments of the compound of Formula VI or VI-I, A is

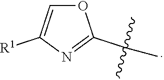

In some embodiments of the compound of Formula VI or VI-I, A is

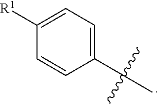

In some embodiments of the compound of Formula VI or VI-I, A is

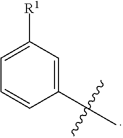

In some embodiments of the compound of Formula VI or VI-I, A is

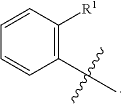

In some embodiments of the compound of Formula VI or VI-I, A is

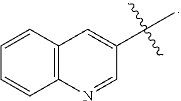 [SMILES: *c1cnc2ccccc2c1]

In some embodiments of the compound of Formula VI or VI-I, A is

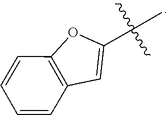 [SMILES: *c1cc2ccccc2o1]

In some embodiments of the compound of Formula VI or VI-I, A is

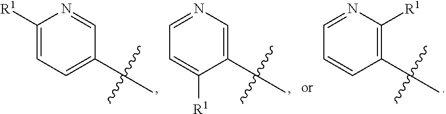

In some embodiments of the compound of Formula VI or VI-I, A is

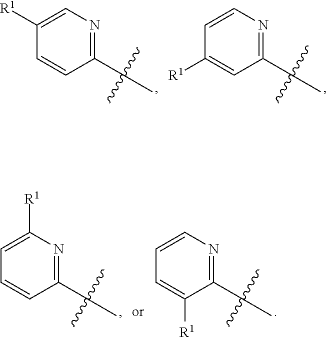

In some embodiments of the compound of Formula VI or VI-I, A is

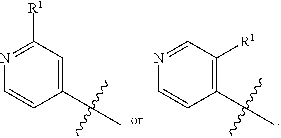

In some embodiments of the compound of Formula VI or VI-I, m=1 and n=1.

In some embodiments of the compound of Formula VI or VI-I, A is

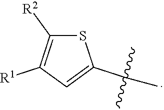

In some embodiments of the compound of Formula VI or VI-I, A is

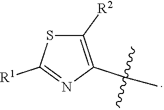

In some embodiments of the compound of Formula VI or VI-I, A is

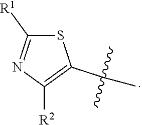

In some embodiments of the compound of Formula VI or VI-I, A is

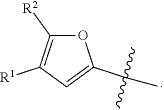

In some embodiments of the compound of Formula VI or VI-I, A is

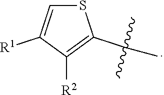

In some embodiments of the compound of Formula VI or VI-I, A is

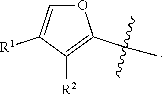

In some embodiments of the compound of Formula VI or VI-I, A is

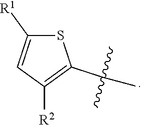

In some embodiments of the compound of Formula VI or VI-I, A is

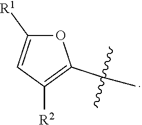

In some embodiments of the compound of Formula VI or VI-I, A is

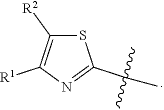

In some embodiments of the compound of Formula VI or VI-I, A is

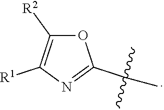

In some embodiments of the compound of Formula VI or VI-I, A is

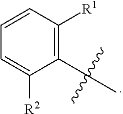

In some embodiments of the compound of Formula VI or VI-I, A is

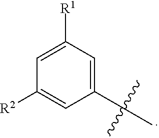

In some embodiments of the compound of Formula VI or VI-I, A

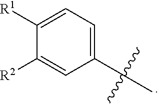

In some embodiments of the compound of Formula VI or VI-I, A is

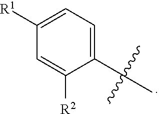

In some embodiments of the compound of Formula VI or VI-I, A is

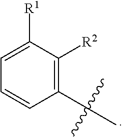

In some embodiments of the compound of Formula VI or VI-I, A is

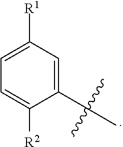

In some embodiments of the compound of Formula VI or VI-I, m=2 and n=1.

In some embodiments of the compound of Formula VI or VI-I, A is

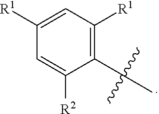

In some embodiments of the compound of Formula VI or VI-I, A is

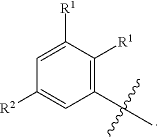

In some embodiments of the compound of Formula VI or VI-I, A

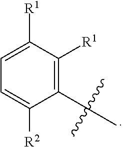

In some embodiments of the compound of Formula VI or VI-I, A is

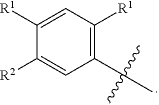

In some embodiments of the compound of Formula VI or VI-I, each of R¹ and R², when present, is independently selected from the group consisting of C₁-C₆ alkyl optionally substituted with one or more hydroxy, halo, oxo, C₁-C₆ alkoxy, or NR⁸R⁹; C₃-C₇ cycloalkyl optionally substituted with one or more hydroxy, halo, oxo, C₁-C₆ alkoxy, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; 3- to 7-membered heterocycloalkyl optionally substituted with one or more hydroxy, halo, oxo, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; C₁-C₆ haloalkyl; C₁-C₆ alkoxy; C₁-C₆ haloalkoxy; halo; CN; CO—C₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO (5- to 10-membered heteroaryl); OCO (3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; S(O₂)NR¹¹R¹²; S(O)C₁-C₆ alkyl; and S(O₂)C₁-C₆ alkyl.

In certain embodiments of the compound of Formula VI or VI-I, R¹ is selected from the group consisting of 1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; S(O₂)CH₃, and S(O₂)NR¹¹R¹².

In certain embodiments of the compound of Formula VI or VI-I, R² is selected from the group consisting of fluoro, chloro, cyano, methyl; methoxy; ethoxy; isopropyl; 1-hydroxy-2-methylpropan-2-yl; 2-hydroxy-2-propyl; hydroxymethyl; 1-hydroxyethyl; 2-hydroxyethyl; 1-hydroxy-2-propyl; 1-hydroxy-1-cyclopropyl; COCH₃; COPh; 2-methoxy-2-propyl; (dimethylamino)methyl; S(O₂)CH₃, and S(O₂)NR¹¹R¹².

In certain embodiments of the compound of Formula VI or VI-I, R¹ is C₁-C₆ alkyl.

In some embodiments of the compound of Formula VI or VI-I, A is

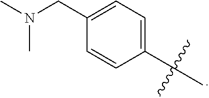 [SMILES: *c1ccc(CN(C)C)cc1]

In some embodiments of the compound of Formula VI or VI-I,

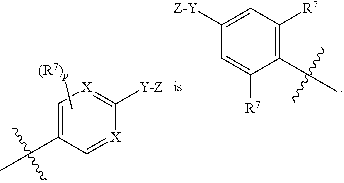

In some embodiments of the compound of Formula VI or VI-I, each R⁷ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl.

In certain embodiments of the compound of Formula VI or VI-I, each R⁷ is independently selected from the group consisting of: C₁-C₆ alkyl, C₃-C₇ cycloalkyl, C₁-C₆ haloalkyl, C₁-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.

In some embodiments of the compound of Formula VI or VI-I, p=2. In certain of these embodiments, each occurrence of R⁸ and R⁹ is independently selected from hydrogen and C₁-C₅ alkyl.

In certain of the foregoing embodiments of Formula VI or VI-I (when p=2 and/or each occurrence of R⁸ and R⁹ is independently selected from hydrogen and C₁-C₅ alkyl), Z is optionally substituted with one or more substituents independently selected from C₁-C₆ alkyl, C₁-C₆ alkoxy, C₆-C₁₀ aryloxy, CN, halo, COOC₁-C₆ alkyl, S(O₂)C₁-C₆ alkyl, 3- to 7-membered heterocycloalkyl, and CONR⁸R⁹, and wherein the C₁-C₆ alkyl or C₁-C₆ alkoxy that Z is substituted with is optionally substituted with one or more hydroxyl, NR⁸R⁹, or C₆-C₁₀ aryl.

As a non-limiting example of the above, Z can be pyrazolyl, pyridinyl, pyrimidinyl, quinolinyl, indolyl, pyrimidin-2-one, thiazolyl, isoxazolyl, or furyl, wherein Z is optionally substituted with one or more substituents independently selected from C₁-C₆ alkyl, C₁-C₆ alkoxy, C₆-C₁₀ aryloxy, CN, halo, COOC₁-C₆ alkyl, S(O₂)C₁-C₆ alkyl, 3- to 7-membered heterocycloalkyl, and CONR⁸R⁹, and wherein the C₁-C₆ alkyl or C₁-C₆ alkoxy that Z is substituted with is optionally substituted with one or more hydroxyl, NR⁸R⁹, or C₆-C₁₀ aryl.

In certain of the foregoing embodiments of Formula VI or VI-I (when p=2 and/or each occurrence of R⁸ and R⁹ is independently selected from hydrogen and C₁-C₅ alkyl), Z is a 5-10-membered monocyclic or bicyclic heterocycloalkyl, wherein Z is optionally substituted with one or more substituents independently selected from C₁-C₆ alkyl, C₁-C₆ alkoxy, C₆-C₁₀ aryloxy, CN, halo, COOC₁-C₆ alkyl, S(O₂)C₁-C₆ alkyl, 3- to 7-membered heterocycloalkyl, and CONR⁸R⁹, and wherein the C₁-C₆ alkyl or C₁-C₆ alkoxy that Z is substituted with is optionally substituted with one or more hydroxyl, NR⁸R⁹, or C₆-C₁₀ aryl.

In certain of the foregoing embodiments of Formula VI or VI-I (when p=2 and/or each occurrence of R⁸ and R⁹ is independently selected from hydrogen and C₁-C₅ alkyl), Z is a C₆-C₁₀ monocyclic or bicyclic aryl, wherein Z is optionally substituted with one or more substituents independently selected from C₁-C₆ alkyl, C₁-C₆ alkoxy, C₆-C₁₀ aryloxy, CN, halo, COOC₁-C₆ alkyl, S(O₂)C₁-C₆ alkyl, 3- to 7-membered heterocycloalkyl, and CONR⁸R⁹, and wherein the C₁-C₆ alkyl or C₁-C₆ alkoxy that Z is substituted with is optionally substituted with one or more hydroxyl, NR⁸R⁹, or C₆-C₁₀ aryl.

As a non-limiting example of the above, Z can be phenyl, naphthyl, or methylenedioxyphenyl, wherein Z is optionally substituted with one or more substituents independently selected from C₁-C₆ alkyl, C₁-C₆ alkoxy, C₆-C₁₀ aryloxy, CN, halo, COOC₁-C₆ alkyl, S(O₂)C₁-C₆ alkyl, 3- to 7-membered heterocycloalkyl, and CONR⁸R⁹, and wherein the C₁-C₆ alkyl or C₁-C₆ alkoxy that Z is substituted with is optionally substituted with one or more hydroxyl, NR⁸R⁹, or C₆-C₁₀ aryl.

In certain of the foregoing embodiments of Formula VI or VI-I (when p=2 and/or each occurrence of R⁸ and R⁹ is independently selected from hydrogen and C₁-C₅ alkyl), Z is optionally substituted with one or more substituents independently selected from C₁-C₆ alkyl, C₁-C₆ alkoxy, C₆-C₁₀ aryloxy, CN, halo, COOC₁-C₆ alkyl, S(O₂)C₁-C₆ alkyl, 3- to 7-membered heterocycloalkyl, and CONR⁸R⁹, and wherein the C₁-C₆ alkyl or C₁-C₆ alkoxy that Z is substituted with is optionally substituted with one or more hydroxyl, NR⁸R⁹, or C₆-C₁₀ aryl.

As a non-limiting example of the above, Z can be cycloalkenyl, wherein Z is optionally substituted with one or more substituents independently selected from C₁-C₆ alkyl, C₁-C₆ alkoxy, C₆-C₁₀ aryloxy, CN, halo, COOC₁-C₆ alkyl, S(O₂)C₁-C₆ alkyl, 3- to 7-membered heterocycloalkyl, and CONR⁸R⁹, and wherein the C₁-C₆ alkyl or C₁-C₆ alkoxy that Z is substituted with is optionally substituted with one or more hydroxyl, NR⁸R⁹, or C₆-C₁₀ aryl.

In some embodiments of the compound of Formula VI or VI-I, each of R⁴ and R⁵ is hydrogen.

In another aspect, an NLRP3 antagonist is a compound selected from the group consisting of the compounds in Table 6 below and pharmaceutically acceptable salts thereof.

TABLE 6

| Com- | Structure | | |
|---|---|---|---|
| pound | | | |
| 101 | | 102 | 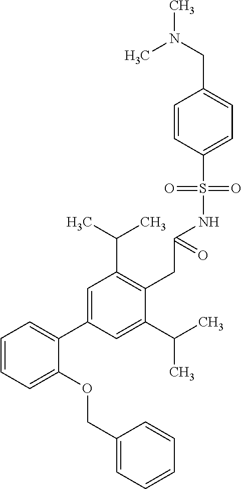 [SMILES: CC(C)c1cc(-c2ccccc2OCc2ccccc2)cc(C(C)C)c1CC(=O)NS(=O)(=O)c1ccc(CN(C)C)cc1] |
| 103 | 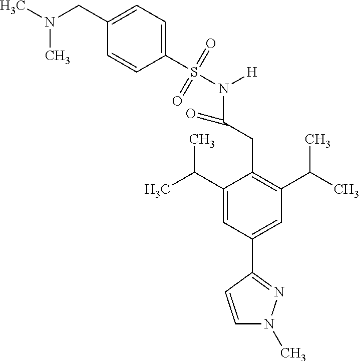 [SMILES: CC(C)c1cc(-c2ccn(C)n2)cc(C(C)C)c1CC(=O)NS(=O)(=O)c1ccc(CN(C)C)cc1] | 104 | 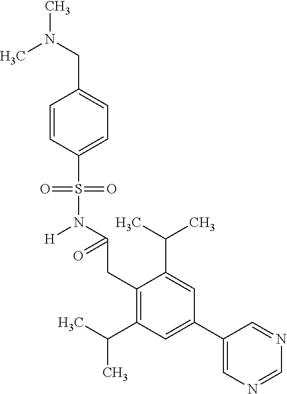 [SMILES: CC(C)c1cc(-c2cncnc2)cc(C(C)C)c1CC(=O)NS(=O)(=O)c1ccc(CN(C)C)cc1] |
| 105 | 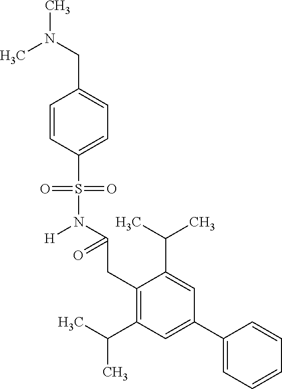 [SMILES: CC(C)c1cc(-c2ccccc2)cc(C(C)C)c1CC(=O)NS(=O)(=O)c1ccc(CN(C)C)cc1] | 106 | 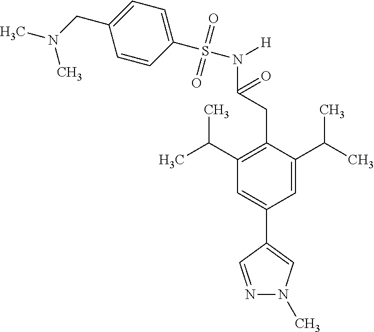 [SMILES: CC(C)c1cc(-c2cnn(C)c2)cc(C(C)C)c1CC(=O)NS(=O)(=O)c1ccc(CN(C)C)cc1] |
| 107 | 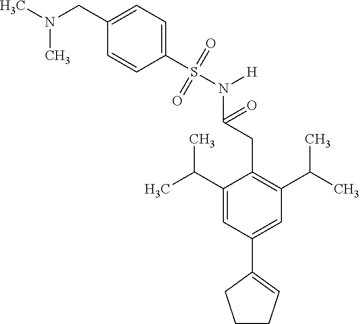 [SMILES: CC(C)c1cc(C2=CCCC2)cc(C(C)C)c1CC(=O)NS(=O)(=O)c1ccc(CN(C)C)cc1] | 108 | 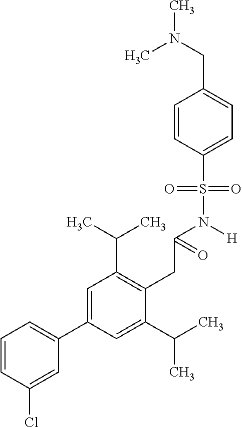 [SMILES: CC(C)c1cc(-c2cccc(Cl)c2)cc(C(C)C)c1CC(=O)NS(=O)(=O)c1ccc(CN(C)C)cc1] |
| 109 | 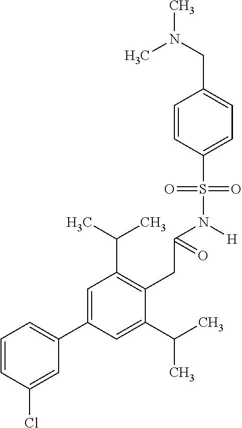 [SMILES: CC(C)c1cc(-c2cccc(Cl)c2)cc(C(C)C)c1CC(=O)NS(=O)(=O)c1ccc(CN(C)C)cc1] | 110 | 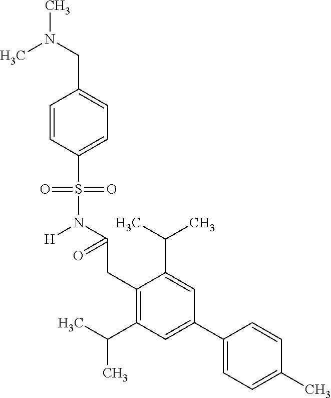 |
| 111 | 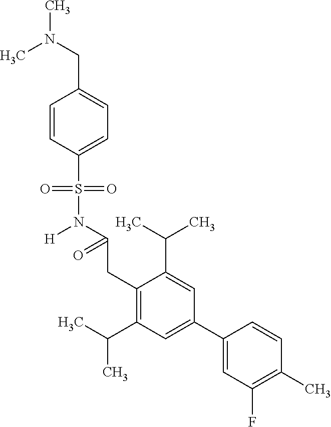 | 112 | 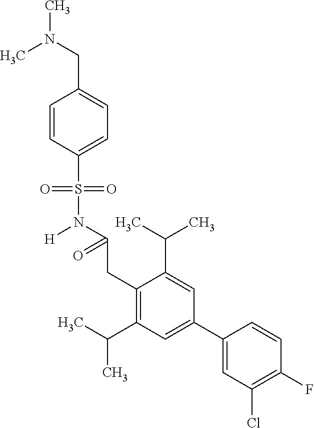 [SMILES: CC(C)c1cc(-c2ccc(F)c(Cl)c2)cc(C(C)C)c1CC(=O)NS(=O)(=O)c1ccc(CN(C)C)cc1] |
| 113 | 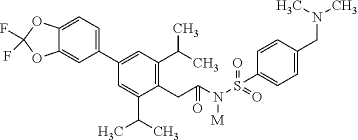 | 114 | 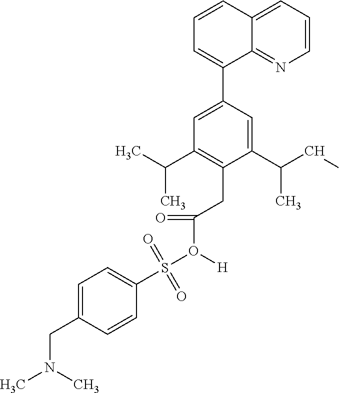 |
| 115 | 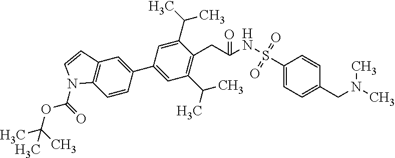 [SMILES: CC(C)c1cc(-c2ccc3c(ccn3C(=O)OC(C)(C)C)c2)cc(C(C)C)c1CC(=O)NS(=O)(=O)c1ccc(CN(C)C)cc1] | 116 | 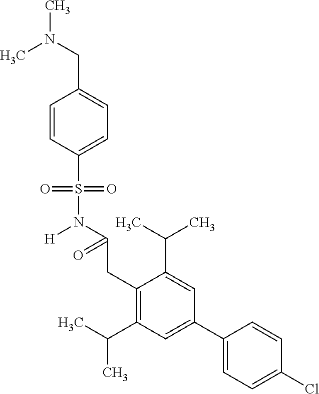 [SMILES: CC(C)c1cc(-c2ccc(Cl)cc2)cc(C(C)C)c1CC(=O)NS(=O)(=O)c1ccc(CN(C)C)cc1] |
| 117 | 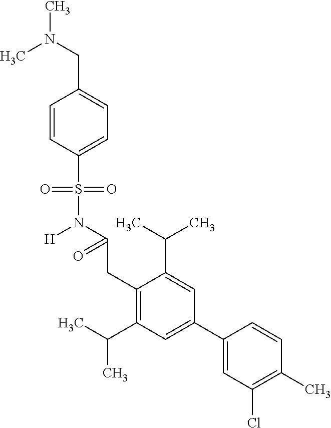 | 118 | 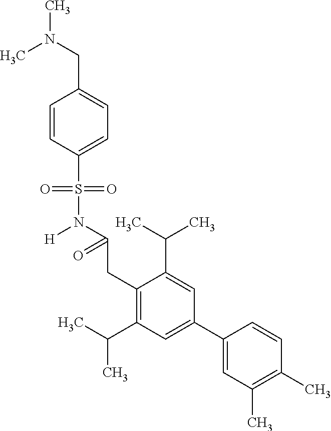 |
| 119 | 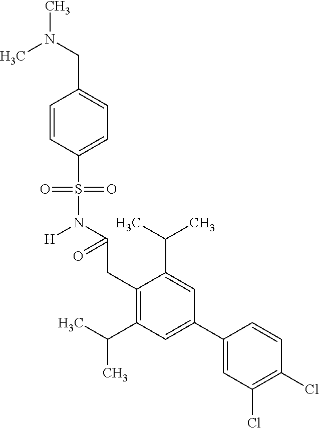 [SMILES: CC(C)c1cc(-c2ccc(Cl)c(Cl)c2)cc(C(C)C)c1CC(=O)NS(=O)(=O)c1ccc(CN(C)C)cc1] | 120 | 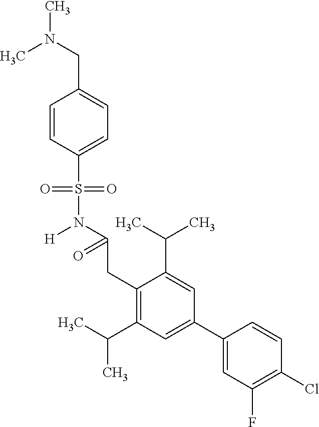 [SMILES: CC(C)c1cc(-c2ccc(Cl)c(F)c2)cc(C(C)C)c1CC(=O)NS(=O)(=O)c1ccc(CN(C)C)cc1] |
| 121 | 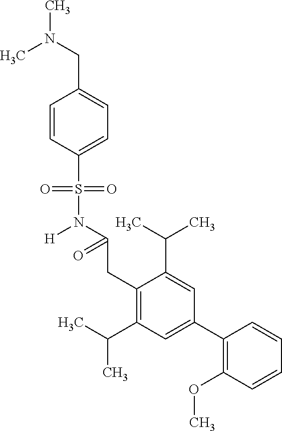 [SMILES: COc1ccccc1-c1cc(C(C)C)c(CC(=O)NS(=O)(=O)c2ccc(CN(C)C)cc2)c(C(C)C)c1] | 122 | 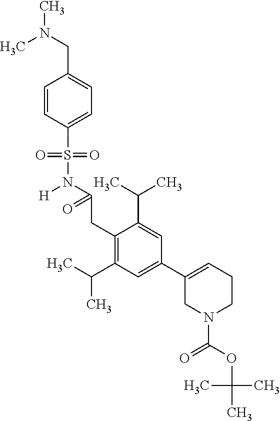 [SMILES: CC(C)c1cc(C2=CCCN(C(=O)OC(C)(C)C)C2)cc(C(C)C)c1CC(=O)NS(=O)(=O)c1ccc(CN(C)C)cc1] |
| 123 | 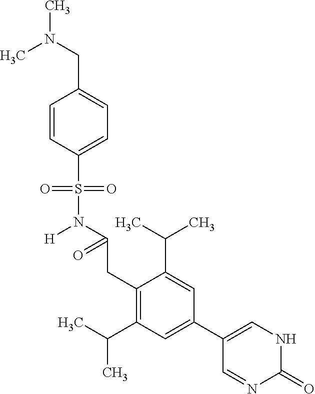 [SMILES: CC(C)c1cc(-c2cnc(=O)[nH]c2)cc(C(C)C)c1CC(=O)NS(=O)(=O)c1ccc(CN(C)C)cc1] | 124 | 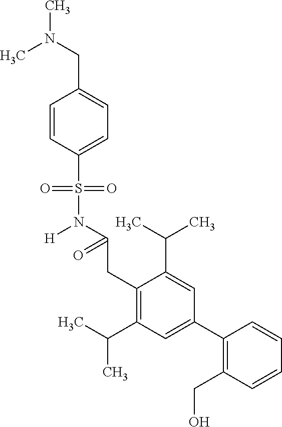 [SMILES: CC(C)c1cc(-c2ccccc2CO)cc(C(C)C)c1CC(=O)NS(=O)(=O)c1ccc(CN(C)C)cc1] |
| 125 | 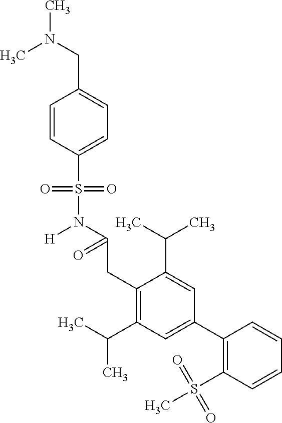 [SMILES: CC(C)c1cc(-c2ccccc2S(C)(=O)=O)cc(C(C)C)c1CC(=O)NS(=O)(=O)c1ccc(CN(C)C)cc1] | 126 | 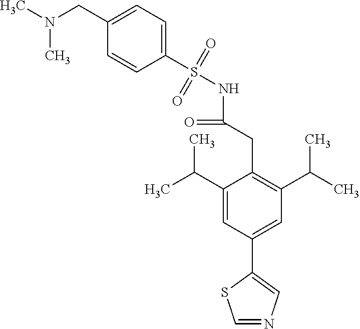 [SMILES: CC(C)c1cc(-c2cncs2)cc(C(C)C)c1CC(=O)NS(=O)(=O)c1ccc(CN(C)C)cc1] |
| 127 | 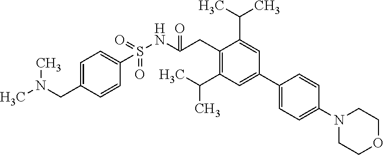 [SMILES: CC(C)c1cc(-c2ccc(N3CCOCC3)cc2)cc(C(C)C)c1CC(=O)NS(=O)(=O)c1ccc(CN(C)C)cc1] | 128 | 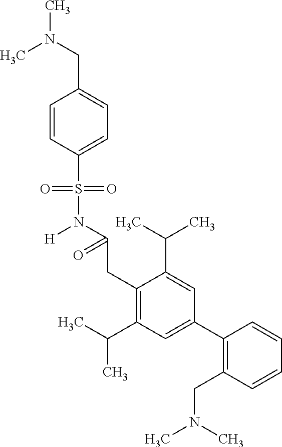 [SMILES: CC(C)c1cc(-c2ccccc2CN(C)C)cc(C(C)C)c1CC(=O)NS(=O)(=O)c1ccc(CN(C)C)cc1] |
| 129 | 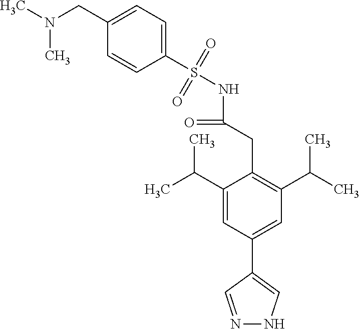 [SMILES: CC(C)c1cc(-c2cn[nH]c2)cc(C(C)C)c1CC(=O)NS(=O)(=O)c1ccc(CN(C)C)cc1] | 130 | 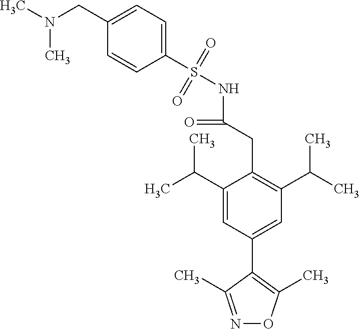 [SMILES: Cc1noc(C)c1-c1cc(C(C)C)c(CC(=O)NS(=O)(=O)c2ccc(CN(C)C)cc2)c(C(C)C)c1] |
| 131 | 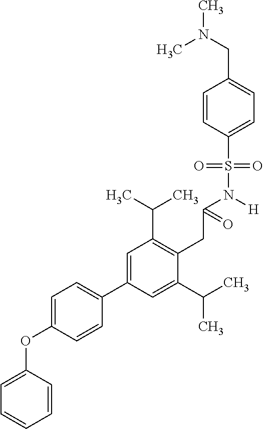 [SMILES: CC(C)c1cc(-c2ccc(Oc3ccccc3)cc2)cc(C(C)C)c1CC(=O)NS(=O)(=O)c1ccc(CN(C)C)cc1] | 132 | 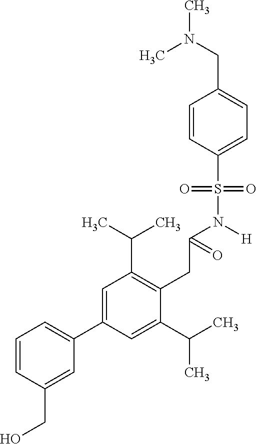 [SMILES: CC(C)c1cc(-c2cccc(CO)c2)cc(C(C)C)c1CC(=O)NS(=O)(=O)c1ccc(CN(C)C)cc1] |
| 133 | 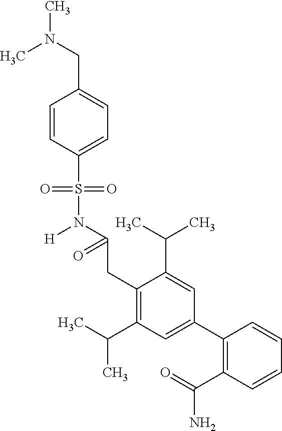 [SMILES: CC(C)c1cc(-c2ccccc2C(N)=O)cc(C(C)C)c1CC(=O)NS(=O)(=O)c1ccc(CN(C)C)cc1] | 134 | 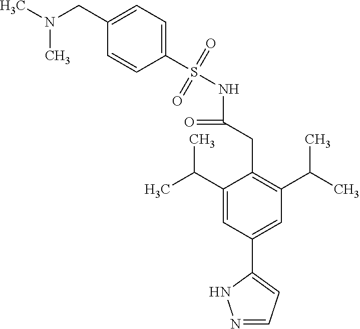 [SMILES: CC(C)c1cc(-c2ccn[nH]2)cc(C(C)C)c1CC(=O)NS(=O)(=O)c1ccc(CN(C)C)cc1] |
| 135 | 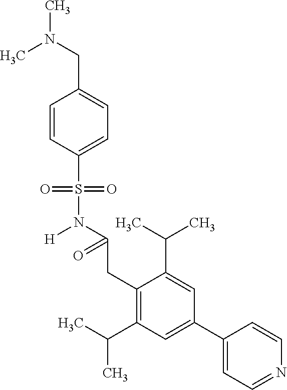 [SMILES: CC(C)c1cc(-c2ccncc2)cc(C(C)C)c1CC(=O)NS(=O)(=O)c1ccc(CN(C)C)cc1] | 136 | 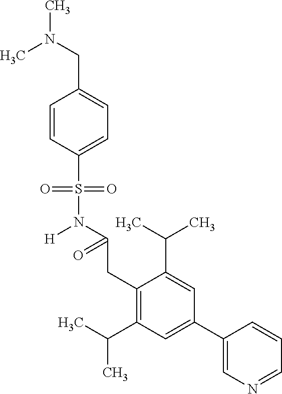 [SMILES: CC(C)c1cc(-c2cccnc2)cc(C(C)C)c1CC(=O)NS(=O)(=O)c1ccc(CN(C)C)cc1] |
| 137 | 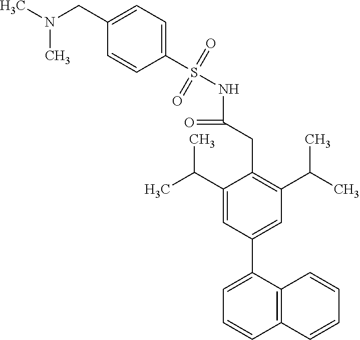 [SMILES: CC(C)c1cc(-c2cccc3ccccc23)cc(C(C)C)c1CC(=O)NS(=O)(=O)c1ccc(CN(C)C)cc1] | 138 | 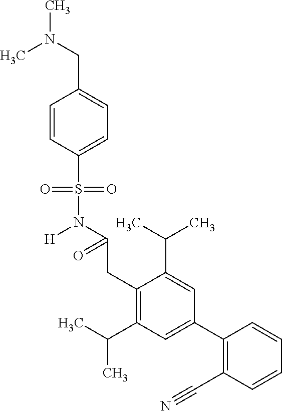 [SMILES: CC(C)c1cc(-c2ccccc2C#N)cc(C(C)C)c1CC(=O)NS(=O)(=O)c1ccc(CN(C)C)cc1] |
| 139 | 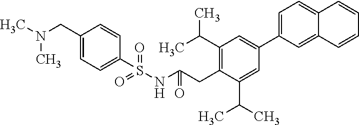 [SMILES: CC(C)c1cc(-c2ccc3ccccc3c2)cc(C(C)C)c1CC(=O)NS(=O)(=O)c1ccc(CN(C)C)cc1] | 140 | 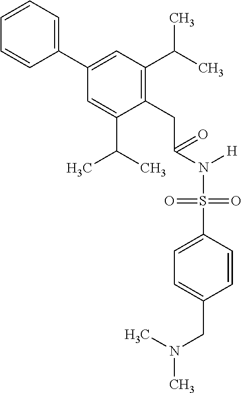 [SMILES: CC(C)c1cc(-c2ccccc2)cc(C(C)C)c1CC(=O)NS(=O)(=O)c1ccc(CN(C)C)cc1] |
| 141 | 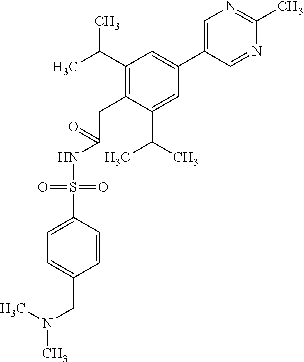 | 142 | 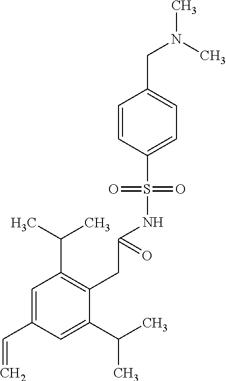 [SMILES: C=Cc1cc(C(C)C)c(CC(=O)NS(=O)(=O)c2ccc(CN(C)C)cc2)c(C(C)C)c1] |
| 143 | 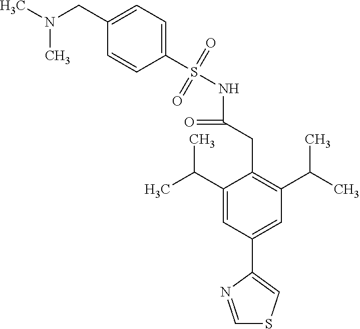 [SMILES: CC(C)c1cc(-c2cscn2)cc(C(C)C)c1CC(=O)NS(=O)(=O)c1ccc(CN(C)C)cc1] | 144 | 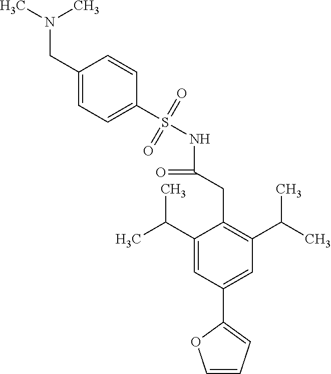 [SMILES: CC(C)c1cc(-c2ccco2)cc(C(C)C)c1CC(=O)NS(=O)(=O)c1ccc(CN(C)C)cc1] |
| 145 | 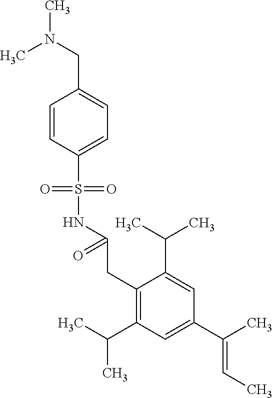 [SMILES: C/C=C(\C)c1cc(C(C)C)c(CC(=O)NS(=O)(=O)c2ccc(CN(C)C)cc2)c(C(C)C)c1] | 146 | 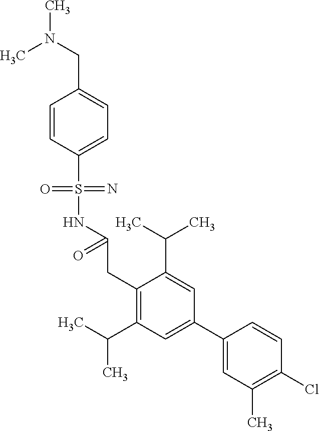 [SMILES: Cc1cc(-c2cc(C(C)C)c(CC(=O)NS(=N)(=O)c3ccc(CN(C)C)cc3)c(C(C)C)c2)ccc1Cl] |
| 147 | 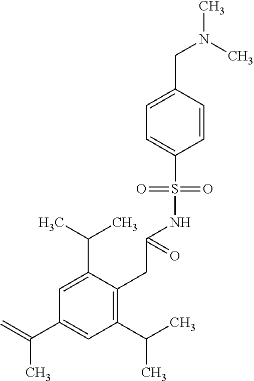 [SMILES: C=C(C)c1cc(C(C)C)c(CC(=O)NS(=O)(=O)c2ccc(CN(C)C)cc2)c(C(C)C)c1] | 148 | 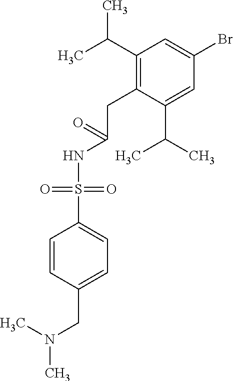 [SMILES: CC(C)c1cc(Br)cc(C(C)C)c1CC(=O)NS(=O)(=O)c1ccc(CN(C)C)cc1] |
| 149 | 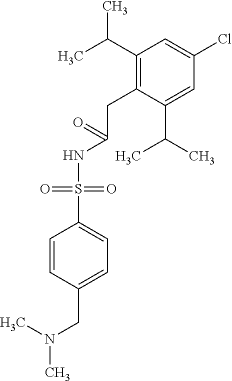 [SMILES: CC(C)c1cc(Cl)cc(C(C)C)c1CC(=O)NS(=O)(=O)c1ccc(CN(C)C)cc1] | 150 | 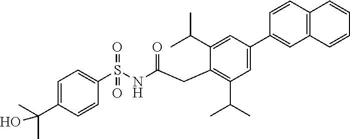 [SMILES: CC(C)c1cc(-c2ccc3ccccc3c2)cc(C(C)C)c1CC(=O)NS(=O)(=O)c1ccc(C(C)(C)O)cc1] |
| 151 | 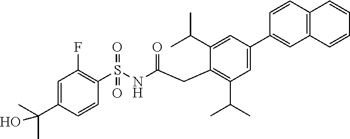 [SMILES: CC(C)c1cc(-c2ccc3ccccc3c2)cc(C(C)C)c1CC(=O)NS(=O)(=O)c1ccc(C(C)(C)O)cc1F] | 152 | 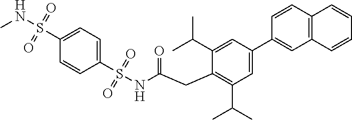 [SMILES: CNS(=O)(=O)c1ccc(S(=O)(=O)NC(=O)Cc2c(C(C)C)cc(-c3ccc4ccccc4c3)cc2C(C)C)cc1] |
| 153 | 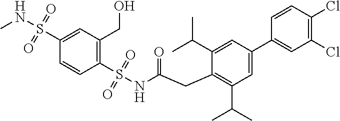 [SMILES: CNS(=O)(=O)c1ccc(S(=O)(=O)NC(=O)Cc2c(C(C)C)cc(-c3ccc(Cl)c(Cl)c3)cc2C(C)C)c(CO)c1] | 154 | 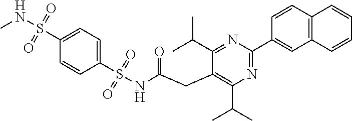 [SMILES: CNS(=O)(=O)c1ccc(S(=O)(=O)NC(=O)Cc2c(C(C)C)nc(-c3ccc4ccccc4c3)nc2C(C)C)cc1] |
| 155 | 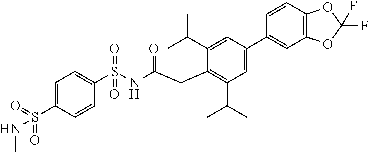 [SMILES: CNS(=O)(=O)c1ccc(S(=O)(=O)NC(=O)Cc2c(C(C)C)cc(-c3ccc4c(c3)OC(F)(F)O4)cc2C(C)C)cc1] | 156 | 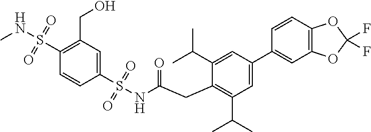 [SMILES: CNS(=O)(=O)c1ccc(S(=O)(=O)NC(=O)Cc2c(C(C)C)cc(-c3ccc4c(c3)OC(F)(F)O4)cc2C(C)C)cc1CO] |
| 157 | 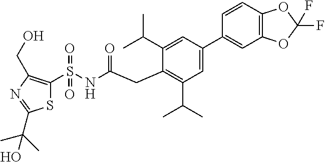 [SMILES: CC(C)c1cc(-c2ccc3c(c2)OC(F)(F)O3)cc(C(C)C)c1CC(=O)NS(=O)(=O)c1sc(C(C)(C)O)nc1CO] | 158 | 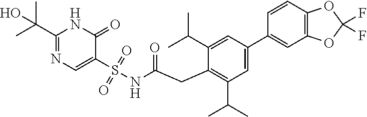 [SMILES: CC(C)c1cc(-c2ccc3c(c2)OC(F)(F)O3)cc(C(C)C)c1CC(=O)NS(=O)(=O)c1cnc(C(C)(C)O)[nH]c1=O] |
| 159 | 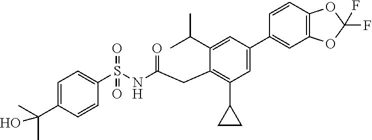 [SMILES: CC(C)c1cc(-c2ccc3c(c2)OC(F)(F)O3)cc(C2CC2)c1CC(=O)NS(=O)(=O)c1ccc(C(C)(C)O)cc1] | 160 | 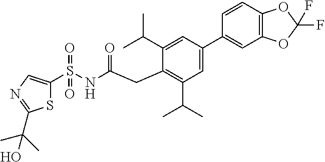 [SMILES: CC(C)c1cc(-c2ccc3c(c2)OC(F)(F)O3)cc(C(C)C)c1CC(=O)NS(=O)(=O)c1cnc(C(C)(C)O)s1] |
| 161 | 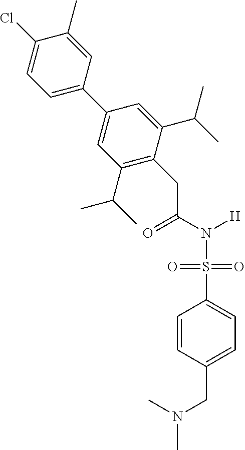 [SMILES: Cc1cc(-c2cc(C(C)C)c(CC(=O)NS(=O)(=O)c3ccc(CN(C)C)cc3)c(C(C)C)c2)ccc1Cl] | | |

Compounds having Formula VI, as well as methods of making and using the same, are further described in U.S. Provisional 62/573,562, filed on Oct. 17, 2017, which is incorporated herein by reference in its entirety.

In one aspect, an NLRP3 antagonist is a compound of Formula VII

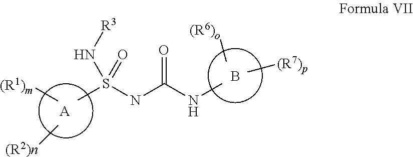

wherein

m=0, 1, or 2

n=0, 1, or 2

o=1 or 2

p=0, 1, 2, or 3

wherein

A is a 5- to 10-membered monocyclic or bicyclic heteroaryl or a C₆-C₁₀ monocyclic or bicyclic aryl;

B is a 5- to 10-membered monocyclic or bicyclic heteroaryl or a C₆-C₁₀ monocyclic or bicyclic aryl;

wherein

at least one R⁶ is ortho to the bond connecting the B ring to the NH(CO) group of Formula VII;

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl; CO (5- to 10-membered heteroaryl); CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3-to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;

or at least one pair of R¹ and R² on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

each of R⁴ and R⁵ is independently selected from hydrogen and C₁-C₆ alkyl;

R¹⁰ is C₁-C₆ alkyl;

each of R⁸ and R⁹ at each occurrence is independently selected from hydrogen, C₁-C₆ alkyl, NH—(C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;

R¹³ is C₁-C₆ alkyl, C₆-C₁₀ aryl, or 5- to 10-membered heteroaryl;

each of R¹¹ and 10² at each occurrence is independently selected from hydrogen and C₁-C₆ alkyl;

R³ is selected from hydrogen, cyano, hydroxy, C₁-C₆ alkoxy, C₁-C₆ alkyl, and

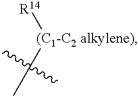

wherein the C₁-C₂ alkylene group is optionally substituted by oxo; R¹⁴ is hydrogen, C₁-C₆ alkyl, 5- to 10-membered monocyclic or bicyclic heteroaryl or C₆-C₁₀ monocyclic or bicyclic aryl, wherein each C₁-C₆ alkyl, aryl or heteroaryl is optionally independently substituted with 1 or 2 R⁶,

or a pharmaceutically acceptable salt thereof.

In some embodiments of the compound of Formula VII, A is a 5- to 6-membered monocyclic heteroaryl optionally substituted with 1 or 2 R¹ and optionally substituted with 1 or 2 R².

In some embodiments of the compound of Formula VII, A is furanyl optionally substituted with 1 or 2 R¹ and optionally substituted with 1 or 2 R².

In some embodiments of the compound of Formula VII, A is thiophenyl optionally substituted with 1 or 2 R¹ and optionally substituted with 1 or 2 R².

In some embodiments of the compound of Formula VII, A is oxazolyl optionally substituted with 1 or 2 R¹ and optionally substituted with 1 or 2 R².

In some embodiments of the compound of Formula VII, A is thiazolyl optionally substituted with 1 or 2 R¹ and optionally substituted with 1 or 2 R².

In some embodiments of the compound of Formula VII, A is phenyl optionally substituted with 1 or 2 R¹ and optionally substituted with 1 or 2 R².

In some embodiments of the compound of Formula VII, m=1 and n=0.

In some embodiments of the compound of Formula VII, A is

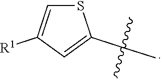

In some embodiments of the compound of Formula VII, A is

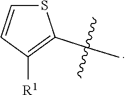

In some embodiments of the compound of Formula VII, A is

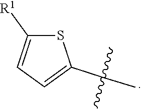

In some embodiments of the compound of Formula VII, A

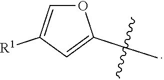

In some embodiments of the compound of Formula VII, A is

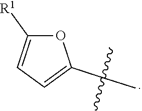

In some embodiments of the compound of Formula VII, A is

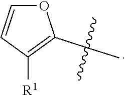

In some embodiments of the compound of Formula VII, A is

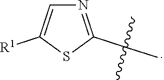

In some embodiments of the compound of Formula VII, A is

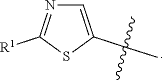

In some embodiments of the compound of Formula VII, A is

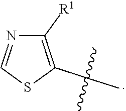

In some embodiments of the compound of Formula VII, A is

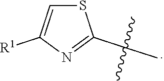

In some embodiments of the compound of Formula VII, A is

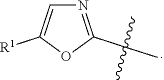

In some embodiments of the compound of Formula VII, A is

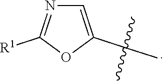

In some embodiments of the compound of Formula VII, A is

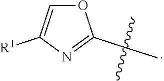

In some embodiments of the compound of Formula VII, A is

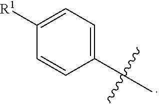

In some embodiments of the compound of Formula VII, A is

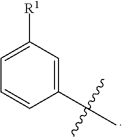

In some embodiments of the compound of Formula VII, A is

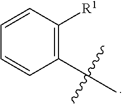

In some embodiments of the compound of Formula VII, A is

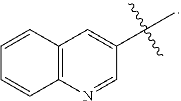 [SMILES: *c1cnc2ccccc2c1]

In some embodiments of the compound of Formula VII, A is

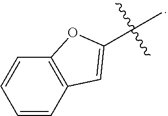 [SMILES: *c1cc2ccccc2o1]

In some embodiments of the compound of Formula VII, A is

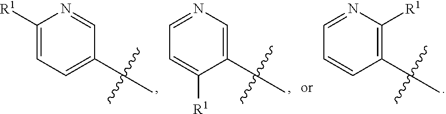

In some embodiments of the compound of Formula VII, A is

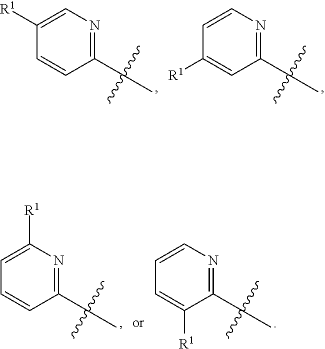

In some embodiments of the compound of Formula VII, A is

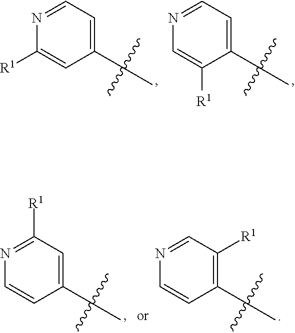

In some embodiments of the compound of Formula VII, m=1 and n=1.

In some embodiments of the compound of Formula VII, A is

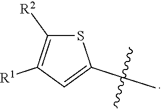

In some embodiments of the compound of Formula VII, A is

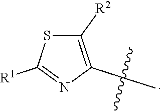

In some embodiments of the compound of Formula VII, A is

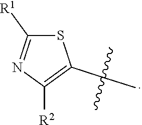

In some embodiments of the compound of Formula VII, A is

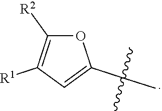

In some embodiments of the compound of Formula VII, A is

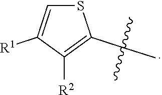

In some embodiments of the compound of Formula VII, A is

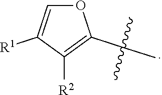

In some embodiments of the compound of Formula VII, A is

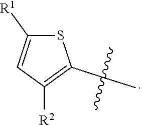

In some embodiments of the compound of Formula VII, A is

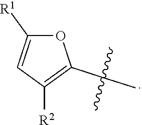

In some embodiments of the compound of Formula VII, A is

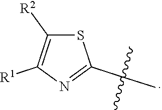

In some embodiments of the compound of Formula VII, A is

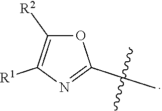

In some embodiments of the compound of Formula VII, A is

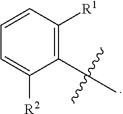

In some embodiments of the compound of Formula VII, A is

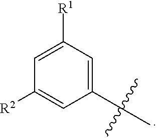

In some embodiments of the compound of Formula VII, A is

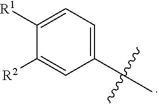

In some embodiments of the compound of Formula VII, A is

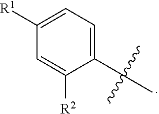

In some embodiments of the compound of Formula VII, A

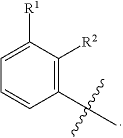

In some embodiments of the compound of Formula VII, A is

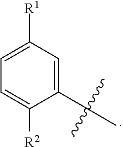

In some embodiments of the compound of Formula VII, m=2 and n=1.

In some embodiments of the compound of Formula VII, A is

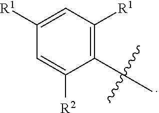

In some embodiments of the compound of Formula VII, A is

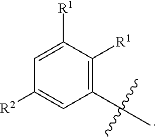

In some embodiments of the compound of Formula VII, A is

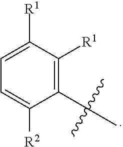

In some embodiments of the compound of Formula VII, A is

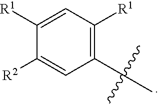

In some embodiments of the compound of Formula VII, each of R¹ and R², when present, is independently selected from the group consisting of C₁-C₆ alkyl optionally substituted with one or more hydroxy, halo, oxo, C₁-C₆ alkoxy, or NR⁸R⁹; C₃-C₇ cycloalkyl optionally substituted with one or more hydroxy, halo, oxo, C₁-C₆ alkoxy, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; 3- to 7-membered heterocycloalkyl optionally substituted with one or more hydroxy, halo, oxo, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; C₁-C₆ haloalkyl; C₁-C₆ alkoxy; C₁-C₆ haloalkoxy; halo; CN; CO—C₁-C₆ alkyl; CO—C₆-C₁₀ aryl; CO (5- to 10-membered heteroaryl); CO₂C₁-C₆ alkyl; CO₂C₃-C₈ cycloalkyl; OCOC₁-C₆ alkyl; OCOC₆-C₁₀ aryl; OCO (5- to 10-membered heteroaryl); OCO (3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; S(O₂)NR¹¹R¹²; S(O)C₁-C₆ alkyl; and S(O₂)C₁-C₆ alkyl.

In certain embodiments of the compound of Formula VII, R¹ is selected from the group consisting of 1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; S(O₂)CH₃, and S(O₂)NR¹¹R¹².

In certain of the foregoing embodiments of Formula VII (when each of R¹ and R², when present, is independently selected from the group consisting of C₁-C₆ alkyl optionally substituted with one or more hydroxy, halo, oxo, C₁-C₆ alkoxy, or NR⁸R⁹; C₃-C₇ cycloalkyl optionally substituted with one or more hydroxy, halo, oxo, C₁-C₆ alkoxy, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; 3- to 7-membered heterocycloalkyl optionally substituted with one or more hydroxy, halo, oxo, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; C₁-C₆ haloalkyl; C₁-C₆ alkoxy; C₁-C₆ haloalkoxy; halo; CN; CO—C₁-C₆ alkyl; CO—C₆-C₁₀ aryl; CO (5- to 10-membered heteroaryl); CO₂C₁-C₆ alkyl; CO₂C₃-C₈ cycloalkyl; OCOC₁-C₆ alkyl; OCOC₆-C₁₀ aryl; OCO (5- to 10-membered heteroaryl); OCO (3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; S(O₂)NR¹¹R¹²; S(O)C₁-C₆ alkyl; and S(O₂)C₁-C₆ alkyl), R² is selected from the group consisting of fluoro, chloro, cyano, methyl; methoxy; ethoxy; isopropyl; 1-hydroxy-2-methylpropan-2-yl; 2-hydroxy-2-propyl; hydroxymethyl; 1-hydroxyethyl; 2-hydroxyethyl; 1-hydroxy-2-propyl; 1-hydroxy-1-cyclopropyl; COCH₃; COPh; 2-methoxy-2-propyl; (dimethylamino)methyl; S(O₂)CH₃, and S(O₂)NR¹¹R¹².

In some embodiments of the compound of Formula VII, B is phenyl substituted with 1 or 2 R⁶ and optionally substituted with 1, 2, or 3 R⁷.

In certain of the foregoing embodiments of Formula VII (when B is phenyl substituted with 1 or 2 R⁶ and optionally substituted with 1, 2, or 3 R⁷), o=2 and p=0.

In certain of the foregoing embodiments of Formula VII (when B is phenyl substituted with 1 or 2 R⁶ and optionally substituted with 1, 2, or 3 R⁷), B is

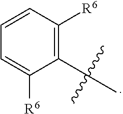

In certain of the foregoing embodiments of Formula VII (when B is

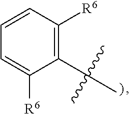

R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl.

As a non-limiting example of the above, each each R⁶ can be independently selected from the group consisting of: C₁-C₆ alkyl, C₃-C₇ cycloalkyl, C₁-C₆ haloalkyl, C₁-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.

In certain embodiments of Formula VII (when B is phenyl substituted with 1 or 2 R⁶ and optionally substituted with 1, 2, or 3 R⁷), o=1 and p=1.

In certain of the foregoing embodiments of Formula VII (when B is phenyl substituted with 1 or 2 R⁶ and optionally substituted with 1, 2, or 3 R⁷), o=2 and p=1.

In certain of the foregoing embodiments of Formula VII (when B is phenyl; o=2; and p=1), B is

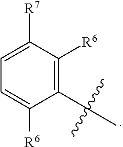

In certain of the foregoing embodiments of Formula VII (when B is

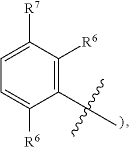

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain embodiments of Formula VII (when B is phenyl; o=2; and p=1), B is

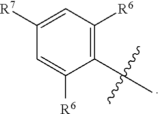

In certain of the foregoing embodiments of Formula VII (when B is

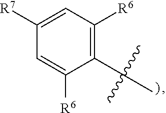

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

In certain of the foregoing embodiments of Formula VII (when B is phenyl substituted with 1 or 2 R⁶ and optionally substituted with 1, 2, or 3 R⁷), o=2 and p=2.

In certain of the foregoing embodiments of Formula VII (when B is phenyl; o=2;

and p=2), B is

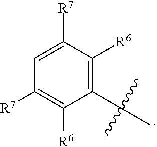

In certain of the foregoing embodiments of Formula VII (when B is

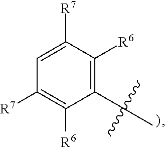

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, NR¹⁰, COOC₁-C₆ alkyl, C₆-C₁₀ aryl, and CONR⁸R⁹.

In certain embodiments of Formula VII (when B is phenyl; o=2; and p=2), B is

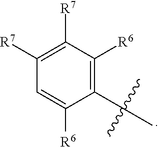

In certain of the foregoing embodiments of Formula VII (when B is

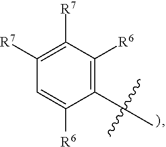

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain embodiments of Formula VII (when B is phenyl substituted with 1 or 2 R⁶ and optionally substituted with 1, 2, or 3 R⁷), o=2 and p=3

In certain of the foregoing embodiments of Formula VII (when B is phenyl; o=2; and p=3), B is

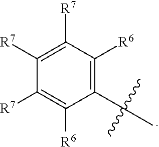

In certain of the foregoing embodiments of Formula VII (when B is

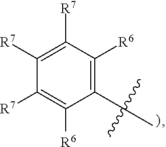

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹;

In some embodiments of the compound of Formula VII, R³ is hydrogen.

In some embodiments of the compound of Formula VII, the sulfur in the moiety S(═O)(NR³)═N— has (S) stereochemistry.

In some embodiments of the compound of Formula VII, the sulfur in the moiety S(═O)(NR³)═N— has (R) stereochemistry.

Compounds having Formula VII, as well as methods of making and using the same, are further described in U.S. Provisional 62/573,894, filed on Oct. 18, 2017; and U.S. Provisional 62/536,271, filed on Jul. 24, 2017, each of which is incorporated herein by reference in its entirety.

In one aspect, an NLRP3 antagonist is a compound of Formula VIII

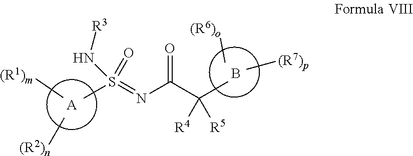

wherein

m=0, 1, or 2

n=0, 1, or 2

o=1 or 2

p=0, 1, 2, or 3

wherein

A is a 5-10-membered monocyclic or bicyclic heteroaryl or a C₆-C₁₀ monocyclic or bicyclic aryl;

B is a 5-10-membered monocyclic or bicyclic heteroaryl or a C₆-C₁₀ monocyclic or bicyclic aryl;

wherein

at least one R⁶ is ortho to the bond connecting the B ring to the C(R⁴R⁵) group of Formula VIII;

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3-to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;

or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

each of R⁴ and R⁵ is independently selected from hydrogen and C₁-C₆ alkyl;

R¹⁰ is C₁-C₆ alkyl;

each of R⁸ and R⁹ at each occurrence is independently selected from hydrogen, C₁-C₆ alkyl, NH—(C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to; R¹³ is C₁-C₆ alkyl, C₆-C₁₀ aryl, or 5- to 10-membered heteroaryl;

each of R¹¹ and R¹² at each occurrence is independently selected from hydrogen and C₁-C₆ alkyl;

R³ is selected from hydrogen, cyano, hydroxy, C₁-C₆ alkoxy, C₁-C₆ alkyl, and

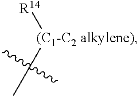

wherein the C₁-C₂ alkylene group is optionally substituted by oxo;

R¹⁴ is hydrogen, C₁-C₆ alkyl, 5-10-membered monocyclic or bicyclic heteroaryl or

C₆-C₁₀ monocyclic or bicyclic aryl, wherein each C₁-C₆ alkyl, aryl or heteroaryl is optionally independently substituted with 1 or 2 R⁶,

or a pharmaceutically acceptable salt thereof.

In some embodiments of the compound of Formula VIII, A is a 5-6-membered monocyclic heteroaryl optionally substituted with 1 or 2 R¹ and optionally substituted with 1 or 2 R².

In some embodiments of the compound of Formula VIII, A is furanyl optionally substituted with 1 or 2 R¹ and optionally substituted with 1 or 2 R².

In some embodiments of the compound of Formula VIII, A is thiophenyl optionally substituted with 1 or 2 R¹ and optionally substituted with 1 or 2 R².

In some embodiments of the compound of Formula VIII, A is oxazolyl optionally substituted with 1 or 2 R¹ and optionally substituted with 1 or 2 R².

In some embodiments of the compound of Formula VIII, A is thiazolyl optionally substituted with 1 or 2 R¹ and optionally substituted with 1 or 2 R².

In some embodiments of the compound of Formula VIII, A is phenyl optionally substituted with 1 or 2 R¹ and optionally substituted with 1 or 2 R².

In some embodiments of the compound of Formula VIII, m=1 and n=0.

In some embodiments of the compound of Formula VIII, A is

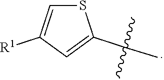

In some embodiments of the compound of Formula VIII, A is

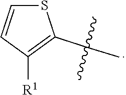

In some embodiments of the compound of Formula VIII, A is

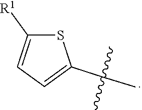

In some embodiments of the compound of Formula VIII, A is

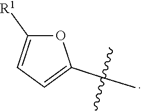

In some embodiments of the compound of Formula VIII, A is

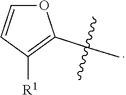

In some embodiments of the compound of Formula VIII, A is

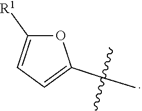

In some embodiments of the compound of Formula VIII, A is

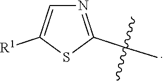

In some embodiments of the compound of Formula VIII, A is

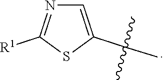

In some embodiments of the compound of Formula VIII, A is

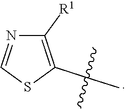

In some embodiments of the compound of Formula VIII, A is

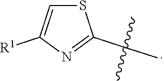

In some embodiments of the compound of Formula VIII, A is

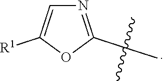

In some embodiments of the compound of Formula VIII, A is

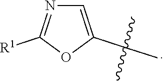

In some embodiments of the compound of Formula VIII, A is

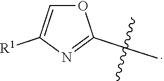

In some embodiments of the compound of Formula VIII, A is

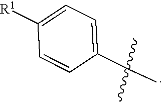

In some embodiments of the compound of Formula VIII, A is

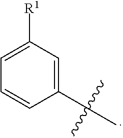

In some embodiments of the compound of Formula VIII, A is

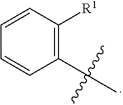

In some embodiments of the compound of Formula VIII, A is

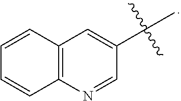 [SMILES: *c1cnc2ccccc2c1]

In some embodiments of the compound of Formula VIII, A is

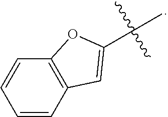 [SMILES: *c1cc2ccccc2o1]

In some embodiments of the compound of Formula VIII, A is

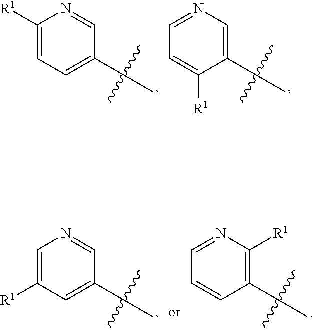

In some embodiments of the compound of Formula VIII, A is

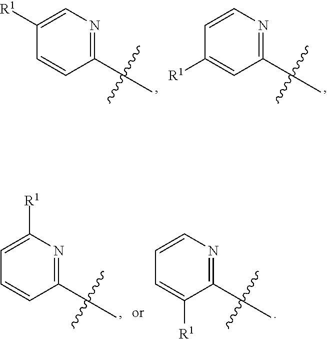

In some embodiments of the compound of Formula VIII, A is

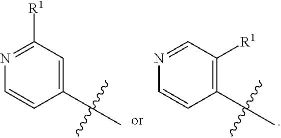

In some embodiments of the compound of Formula VIII, m=1 and n=1.

In some embodiments of the compound of Formula VIII, A is

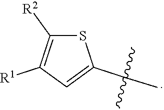

In some embodiments of the compound of Formula VIII, A is

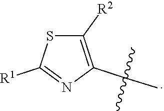

In some embodiments of the compound of Formula VIII, A is

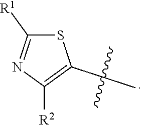

In some embodiments of the compound of Formula VIII, A is

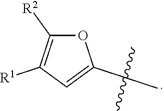

In some embodiments of the compound of Formula VIII, A is

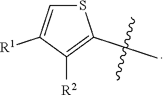

In some embodiments of the compound of Formula VIII, A is

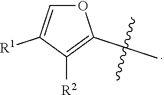

In some embodiments of the compound of Formula VIII, A is

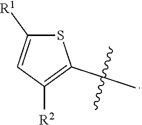

In some embodiments of the compound of Formula VIII, A is

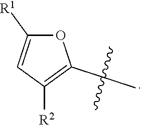

In some embodiments of the compound of Formula VIII, A is

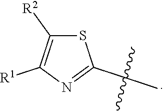

In some embodiments of the compound of Formula VIII, A is

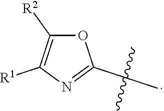

In some embodiments of the compound of Formula VIII, A is

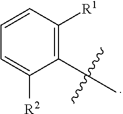

In some embodiments of the compound of Formula VIII, A is

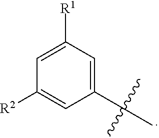

In some embodiments of the compound of Formula VIII, A is

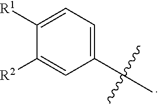

In some embodiments of the compound of Formula VIII, A is

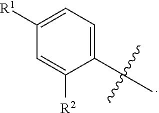

In some embodiments of the compound of Formula VIII, A is

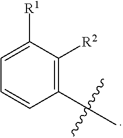

In some embodiments of the compound of Formula VIII, A is

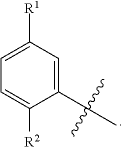

In some embodiments of the compound of Formula VIII, m=2 and n=1.

In some embodiments of the compound of Formula VIII, A is

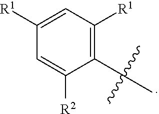

In some embodiments of the compound of Formula VIII, A is

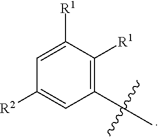

In some embodiments of the compound of Formula VIII, A is

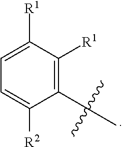

In some embodiments of the compound of Formula VIII, A is

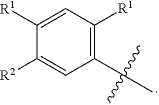

In some embodiments of the compound of Formula VIII, each of R¹ and R², when present, is independently selected from the group consisting of C₁-C₆ alkyl optionally substituted with one or more hydroxy, halo, oxo, C₁-C₆ alkoxy, or NR⁸R⁹; C₃-C₇ cycloalkyl optionally substituted with one or more hydroxy, halo, oxo, C₁-C₆ alkoxy, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; 3- to 7-membered heterocycloalkyl optionally substituted with one or more hydroxy, halo, oxo, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; C₁-C₆ haloalkyl; C₁-C₆ alkoxy; C₁-C₆ haloalkoxy; halo; CN; CO—C₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO (5- to 10-membered heteroaryl); OCO (3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; S(O₂)NR¹¹R¹²; S(O)C₁-C₆ alkyl; and S(O₂)C₁-C₆ alkyl.

In some embodiments of the compound of Formula VIII, R¹ is selected from the group consisting of 1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; S(O₂)CH₃, and S(O₂)NR¹¹R¹².

In certain of the foregoing embodiments of Formula VIII (when each of R¹ and R², when present, is independently selected from the group consisting of C₁-C₆ alkyl optionally substituted with one or more hydroxy, halo, oxo, C₁-C₆ alkoxy, or NR⁸R⁹; C₃-C₇ cycloalkyl optionally substituted with one or more hydroxy, halo, oxo, C₁-C₆ alkoxy, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; 3- to 7-membered heterocycloalkyl optionally substituted with one or more hydroxy, halo, oxo, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; C₁-C₆ haloalkyl; C₁-C₆ alkoxy; C₁-C₆ haloalkoxy; halo; CN; CO—C₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO (5- to 10-membered heteroaryl); OCO (3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; S(O₂)NR¹¹R¹²; S(O)C₁-C₆ alkyl; and S(O₂)C₁-C₆ alkyl), R² is selected from the group consisting of fluoro, chloro, cyano, methyl; methoxy; ethoxy; isopropyl; 1-hydroxy-2-methylpropan-2-yl; 2-hydroxy-2-propyl; hydroxymethyl; 1-hydroxy ethyl; 2-hydroxyethyl; 1-hydroxy-2-propyl; 1-hydroxy-1-cyclopropyl; COCH₃; COPh; 2-methoxy-2-propyl; (dimethylamino)methyl; S(O₂)CH₃, and S(O₂)N—R¹¹R¹².

In some embodiments of the compound of Formula VIII, B is phenyl substituted with 1 or 2 R⁶ and optionally substituted with 1, 2, or 3 R⁷.

In certain of the foregoing embodiments of Formula VIII (when B is phenyl substituted with 1 or 2 R⁶ and optionally substituted with 1, 2, or 3 R⁷), o=2 and p=0.

In certain of the foregoing embodiments of Formula VIII (when B is phenyl

substituted with 1 or 2 R⁶ and optionally substituted with 1, 2, or 3 R⁷), B is

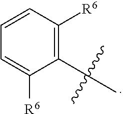

In certain of the foregoing embodiments of Formula VIII (when B is

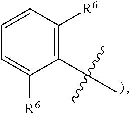

each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl.

In certain of the foregoing embodiments of Formula VIII (when B is

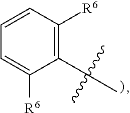

each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.

In certain embodiments of Formula VIII (when B is phenyl substituted with 1 or 2 R⁶ and optionally substituted with 1, 2, or 3 R⁷), o=1 and p=1.

In certain of the foregoing embodiments of Formula VIII (when B is phenyl substituted with 1 or 2 R⁶ and optionally substituted with 1, 2, or 3 R⁷), o=2 and p=1.

In certain of the foregoing embodiments of Formula VIII (when B is phenyl; o=2; and p=1), B is

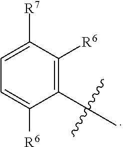

In certain of the foregoing embodiments of Formula VIII (when B is

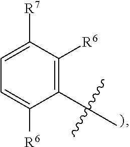

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R₉, ═NR¹⁰, COOC₁-C₆ alkyl, C₆-C₁₀ aryl, and CONR⁸R⁹.

In certain embodiments of Formula VIII (when B is phenyl; o=2; and p=1), B is

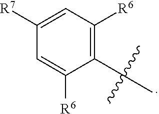

In certain of the foregoing embodiments of Formula VIII (when B is

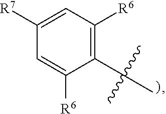

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

In certain embodiments of Formula VIII (when B is phenyl substituted with 1 or 2 R⁶ and optionally substituted with 1, 2, or 3 R⁷), o=2 and p=2.

In certain of the foregoing embodiments of Formula VIII (when B is phenyl; o=2; and p=2), B is

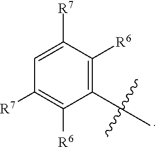

In certain of the foregoing embodiments of Formula VIII (when B is

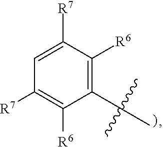

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain embodiments of Formula VIII (when B is phenyl; o=2; and p=2), B is

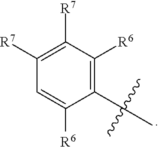

In certain of the foregoing embodiments of Formula VIII (when B is

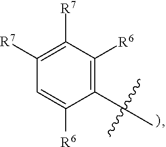

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain embodiments of Formula VIII (when B is phenyl substituted with 1 or 2 R⁶ and optionally substituted with 1, 2, or 3 R⁷), o=2 and p=3

In certain of the foregoing embodiments of Formula VIII (when B is phenyl; o=2; and p=3), B is

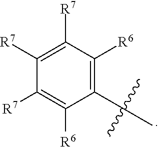

In certain of the foregoing embodiments of Formula VIII (when B is

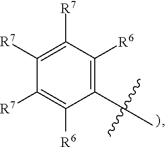

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹;

In some embodiments of the compound of Formula VIII, each of R⁴ and R⁵ is hydrogen.

In some embodiments of the compound of Formula VIII, R³ is hydrogen.

In another aspect, an NLRP3 antagonist is a compound selected from a group consisting of the compounds in Table 7 below and pharmaceutically acceptable salts thereof.

TABLE 7

| Com- | |
|---|---|
| pound | Structure |
| 101 | 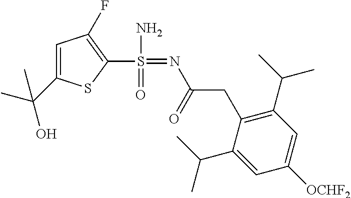 [SMILES: CC(C)c1cc(OC(F)F)cc(C(C)C)c1CC(=O)N=S(N)(=O)c1sc(C(C)(C)O)cc1F] |
| 101a | 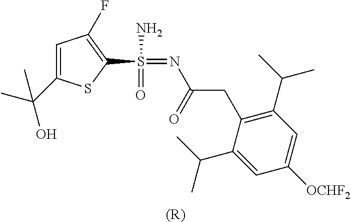 |
| 101b | 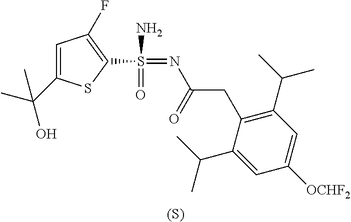 |
| 102 | 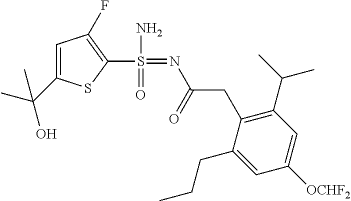 [SMILES: CCCc1cc(OC(F)F)cc(C(C)C)c1CC(=O)N=S(N)(=O)c1sc(C(C)(C)O)cc1F] |
| 103 | 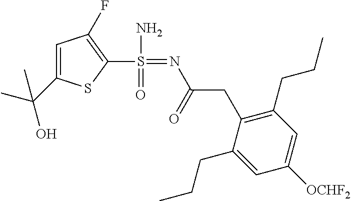 [SMILES: CCCc1cc(OC(F)F)cc(CCC)c1CC(=O)N=S(N)(=O)c1sc(C(C)(C)O)cc1F] |
| 104 | 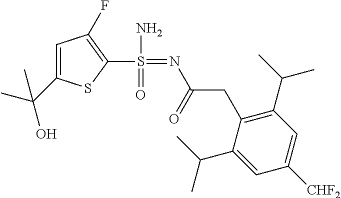 [SMILES: CC(C)c1cc(C(F)F)cc(C(C)C)c1CC(=O)N=S(N)(=O)c1sc(C(C)(C)O)cc1F] |
| 104a | 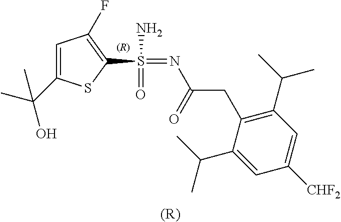 |
| 104b | 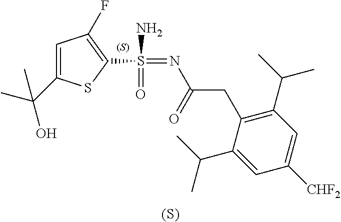 |
| 105 | 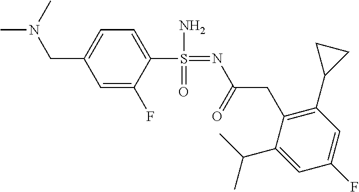 [SMILES: CC(C)c1cc(F)cc(C2CC2)c1CC(=O)N=S(N)(=O)c1ccc(CN(C)C)cc1F] |
| 106 | 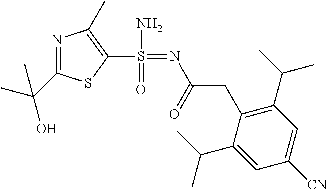 [SMILES: Cc1nc(C(C)(C)O)sc1S(N)(=O)=NC(=O)Cc1c(C(C)C)cc(C#N)cc1C(C)C] |
| 106a | 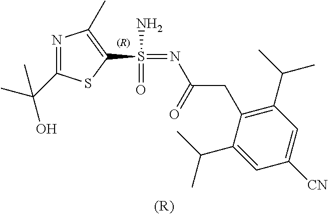 |
| 106b | 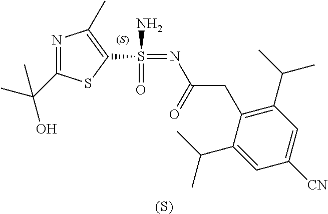 |
| 107 | 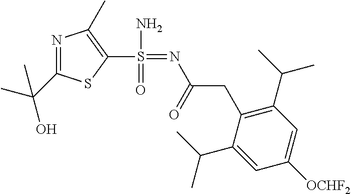 [SMILES: Cc1nc(C(C)(C)O)sc1S(N)(=O)=NC(=O)Cc1c(C(C)C)cc(OC(F)F)cc1C(C)C] |
| 107a | 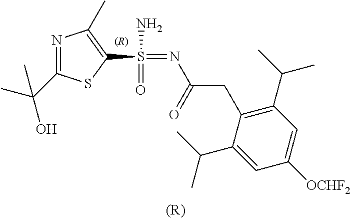 |
| 107b | 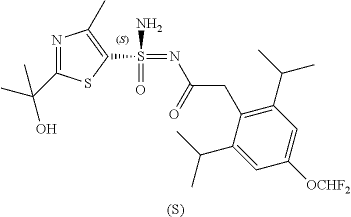 |
| 108 | 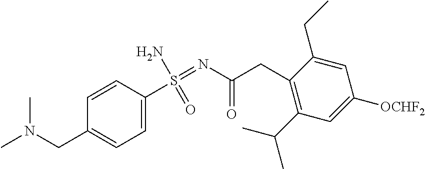 [SMILES: CCc1cc(OC(F)F)cc(C(C)C)c1CC(=O)N=S(N)(=O)c1ccc(CN(C)C)cc1] |
| 109 | 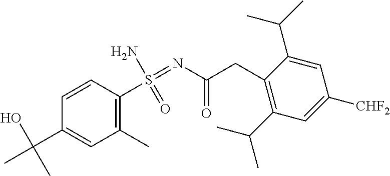 [SMILES: Cc1cc(C(C)(C)O)ccc1S(N)(=O)=NC(=O)Cc1c(C(C)C)cc(C(F)F)cc1C(C)C] |
| 110 | 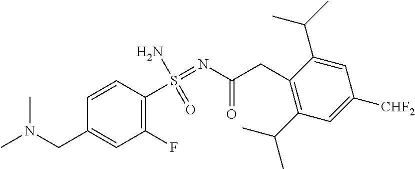 [SMILES: CC(C)c1cc(C(F)F)cc(C(C)C)c1CC(=O)N=S(N)(=O)c1ccc(CN(C)C)cc1F] |
| 110a | 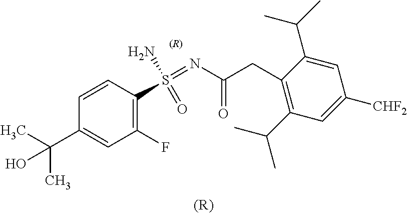 |
| 110b | 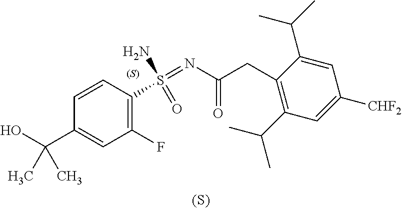 |
| 111 | 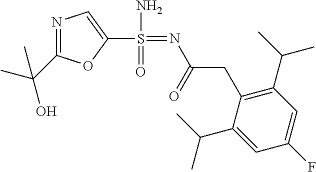 [SMILES: CC(C)c1cc(F)cc(C(C)C)c1CC(=O)N=S(N)(=O)c1cnc(C(C)(C)O)o1] |
| 112 | 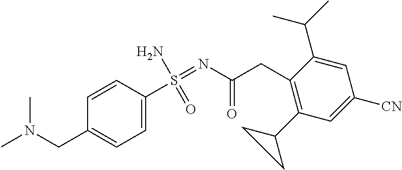 [SMILES: CC(C)c1cc(C#N)cc(C2CC2)c1CC(=O)N=S(N)(=O)c1ccc(CN(C)C)cc1] |
| 112a | 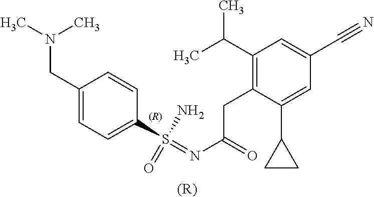 |
| 112b | 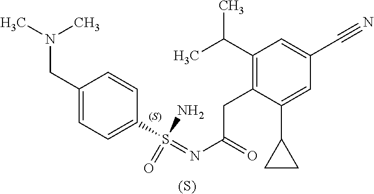 |
| 113 | 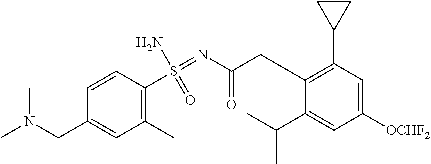 [SMILES: Cc1cc(CN(C)C)ccc1S(N)(=O)=NC(=O)Cc1c(C(C)C)cc(OC(F)F)cc1C1CC1] |
| 114 | 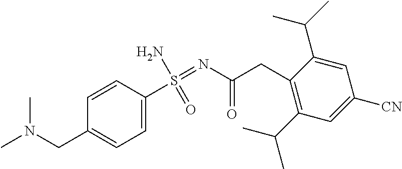 [SMILES: CC(C)c1cc(C#N)cc(C(C)C)c1CC(=O)N=S(N)(=O)c1ccc(CN(C)C)cc1] |
| 114a | 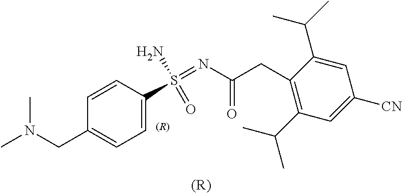 |
| 114b | 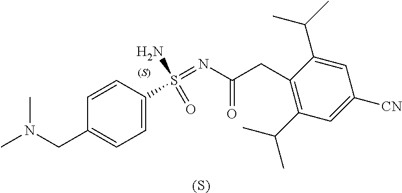 |
| 115 | 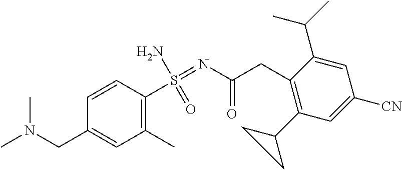 [SMILES: Cc1cc(CN(C)C)ccc1S(N)(=O)=NC(=O)Cc1c(C(C)C)cc(C#N)cc1C1CC1] |
| 116 | 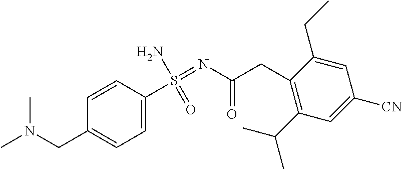 [SMILES: CCc1cc(C#N)cc(C(C)C)c1CC(=O)N=S(N)(=O)c1ccc(CN(C)C)cc1] |
| 116a | 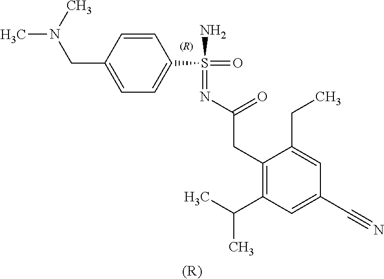 |
| 116b | 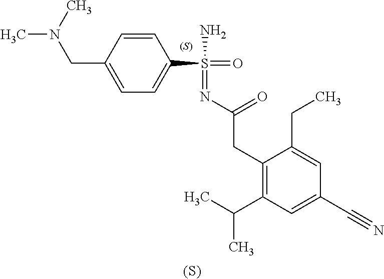 |
| 117 | 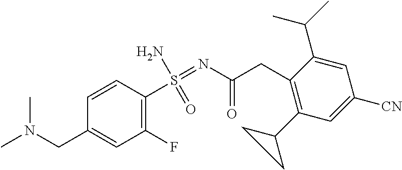 [SMILES: CC(C)c1cc(C#N)cc(C2CC2)c1CC(=O)N=S(N)(=O)c1ccc(CN(C)C)cc1F] |
| 117a | 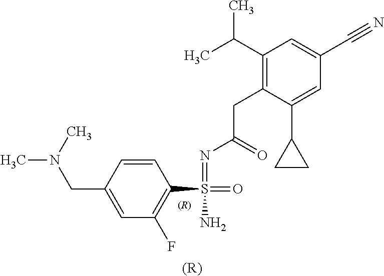 |
| 117b | 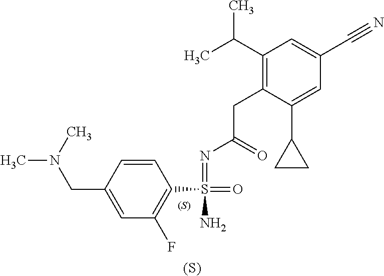 |
| 118 | 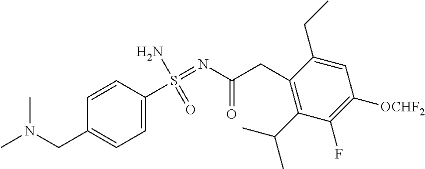 [SMILES: CCc1cc(OC(F)F)c(F)c(C(C)C)c1CC(=O)N=S(N)(=O)c1ccc(CN(C)C)cc1] |
| 119 | 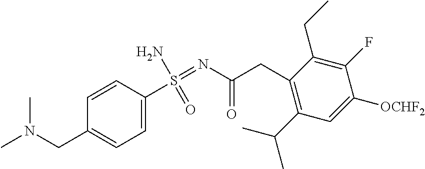 [SMILES: CCc1c(F)c(OC(F)F)cc(C(C)C)c1CC(=O)N=S(N)(=O)c1ccc(CN(C)C)cc1] |
| 120 | 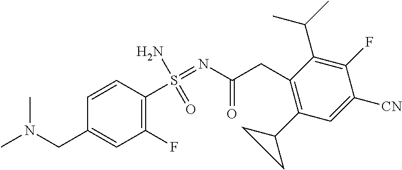 [SMILES: CC(C)c1c(F)c(C#N)cc(C2CC2)c1CC(=O)N=S(N)(=O)c1ccc(CN(C)C)cc1F] |
| 121 | 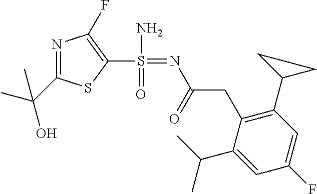 [SMILES: CC(C)c1cc(F)cc(C2CC2)c1CC(=O)N=S(N)(=O)c1sc(C(C)(C)O)nc1F] |
| 122 | 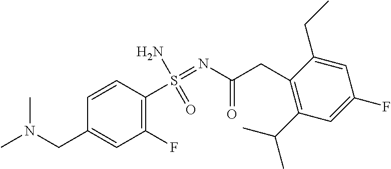 [SMILES: CCc1cc(F)cc(C(C)C)c1CC(=O)N=S(N)(=O)c1ccc(CN(C)C)cc1F] |
| 123 | 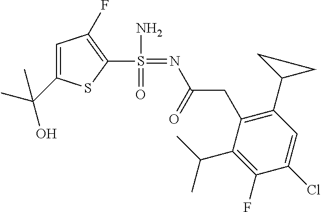 [SMILES: CC(C)c1c(F)c(Cl)cc(C2CC2)c1CC(=O)N=S(N)(=O)c1sc(C(C)(C)O)cc1F] |
| 123a | 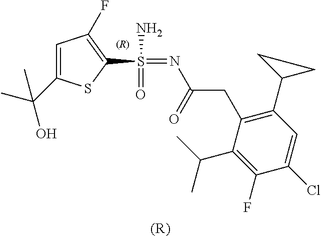 |
| 123b | 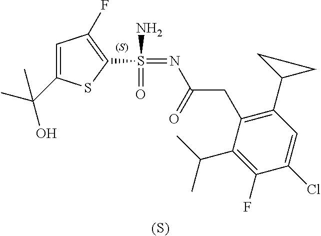 |
| 124 | 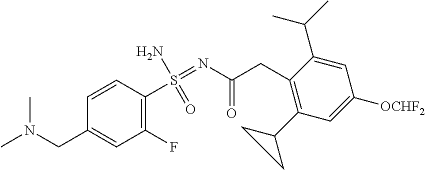 [SMILES: CC(C)c1cc(OC(F)F)cc(C2CC2)c1CC(=O)N=S(N)(=O)c1ccc(CN(C)C)cc1F] |
| 125 | 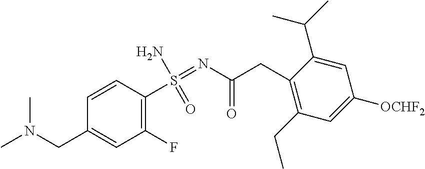 [SMILES: CCc1cc(OC(F)F)cc(C(C)C)c1CC(=O)N=S(N)(=O)c1ccc(CN(C)C)cc1F] |
| 126 | 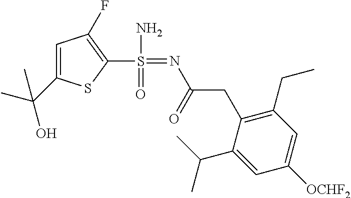 [SMILES: CCc1cc(OC(F)F)cc(C(C)C)c1CC(=O)N=S(N)(=O)c1sc(C(C)(C)O)cc1F] |
| 126a | 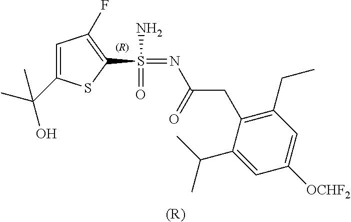 |
| 126b | 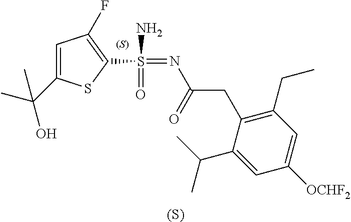 |
| 127 | 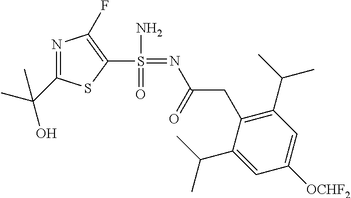 [SMILES: CC(C)c1cc(OC(F)F)cc(C(C)C)c1CC(=O)N=S(N)(=O)c1sc(C(C)(C)O)nc1F] |
| 128 | 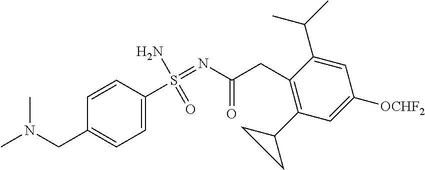 [SMILES: CC(C)c1cc(OC(F)F)cc(C2CC2)c1CC(=O)N=S(N)(=O)c1ccc(CN(C)C)cc1] |
| 129 | 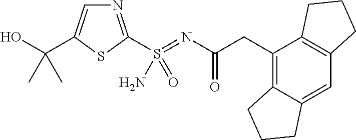 [SMILES: CC(C)(O)c1cnc(S(N)(=O)=NC(=O)Cc2c3c(cc4c2CCC4)CCC3)s1] |
| 129a | 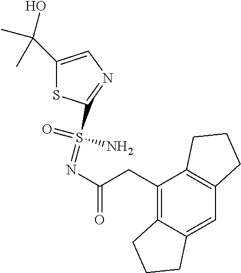 [SMILES: CC(C)(O)c1cnc([S@](N)(=O)=NC(=O)Cc2c3c(cc4c2CCC4)CCC3)s1] |
| 129b | 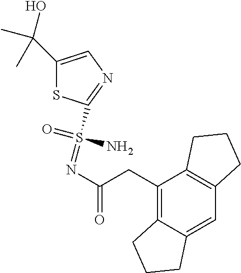 [SMILES: CC(C)(O)c1cnc([S@@](N)(=O)=NC(=O)Cc2c3c(cc4c2CCC4)CCC3)s1] |
| 130 | 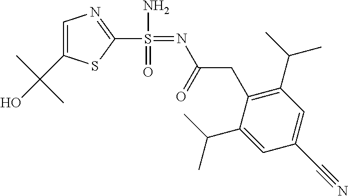 [SMILES: CC(C)c1cc(C#N)cc(C(C)C)c1CC(=O)N=S(N)(=O)c1ncc(C(C)(C)O)s1] |
| 130a | 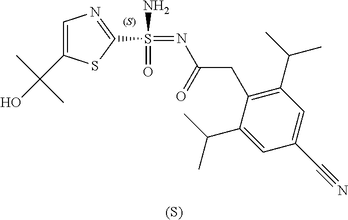 |
| 130b | 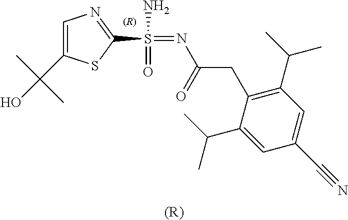 |
| 131 | 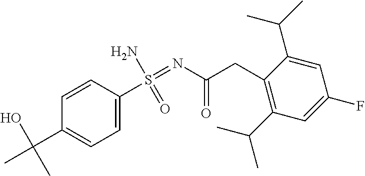 [SMILES: CC(C)c1cc(F)cc(C(C)C)c1CC(=O)N=S(N)(=O)c1ccc(C(C)(C)O)cc1] |
| 131a | 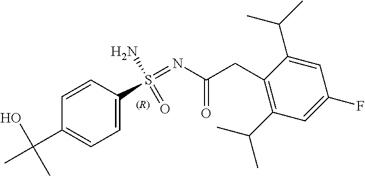 |
| 131b | 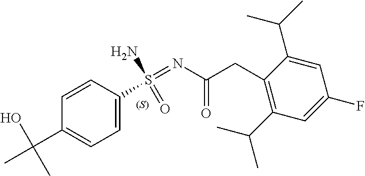 |
| 132 | 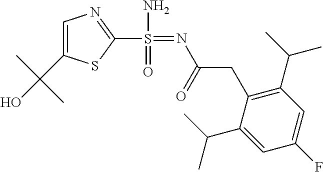 [SMILES: CC(C)c1cc(F)cc(C(C)C)c1CC(=O)N=S(N)(=O)c1ncc(C(C)(C)O)s1] |
| 132a | 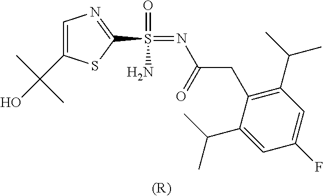 [SMILES: CC(C)c1cc(F)cc(C(C)C)c1CC(=O)N=[S@](N)(=O)c1ncc(C(C)(C)O)s1 ; "(R)"] |
| 132b | 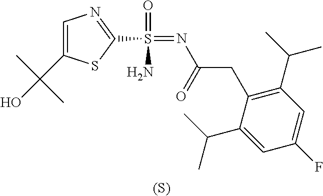 [SMILES: CC(C)c1cc(F)cc(C(C)C)c1CC(=O)N=[S@@](N)(=O)c1ncc(C(C)(C)O)s1 ; "(S)"] |
| 133 | 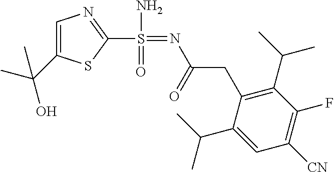 [SMILES: CC(C)c1cc(C#N)c(F)c(C(C)C)c1CC(=O)N=S(N)(=O)c1ncc(C(C)(C)O)s1] |
| 133a | 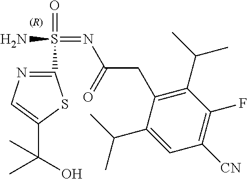 |
| 133b | 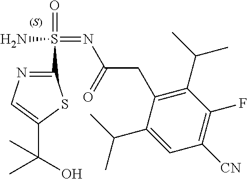 |
| 134 | 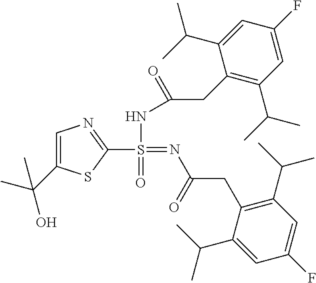 [SMILES: CC(C)c1cc(F)cc(C(C)C)c1CC(=O)N=S(=O)(NC(=O)Cc1c(C(C)C)cc(F)cc1C(C)C)c1ncc(C(C)(C)O)s1] |
| 135 | 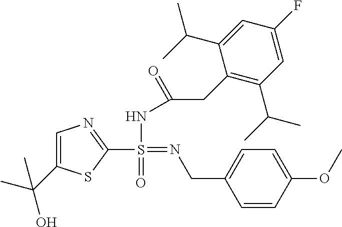 [SMILES: COc1ccc(CN=S(=O)(NC(=O)Cc2c(C(C)C)cc(F)cc2C(C)C)c2ncc(C(C)(C)O)s2)cc1] |
| 136 | 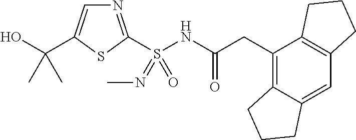 [SMILES: CN=S(=O)(NC(=O)Cc1c2c(cc3c1CCC3)CCC2)c1ncc(C(C)(C)O)s1] |
| 137 | 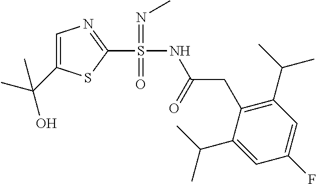 [SMILES: CN=S(=O)(NC(=O)Cc1c(C(C)C)cc(F)cc1C(C)C)c1ncc(C(C)(C)O)s1] |
| 138 | 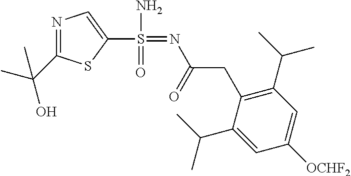 [SMILES: CC(C)c1cc(OC(F)F)cc(C(C)C)c1CC(=O)N=S(N)(=O)c1cnc(C(C)(C)O)s1] |
| 138a | 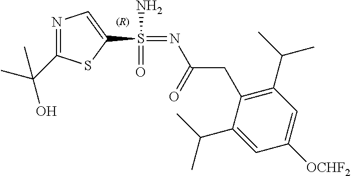 |
| 138b | 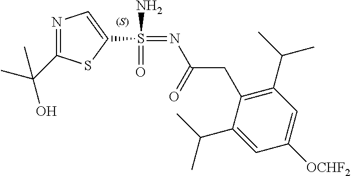 |
| 139 | 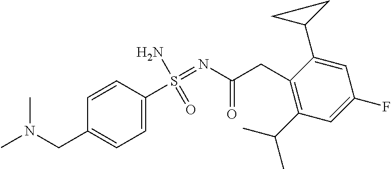 [SMILES: CC(C)c1cc(F)cc(C2CC2)c1CC(=O)N=S(N)(=O)c1ccc(CN(C)C)cc1] |
| 139a | 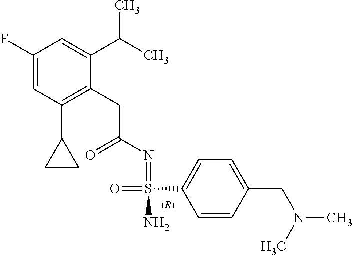 |
| 139b | 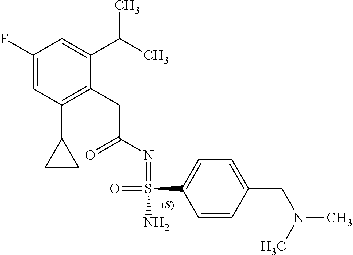 |
| 140 | 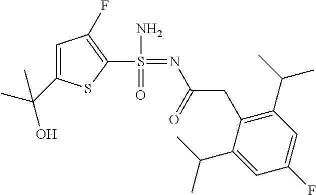 [SMILES: CC(C)c1cc(F)cc(C(C)C)c1CC(=O)N=S(N)(=O)c1sc(C(C)(C)O)cc1F] |
| 140a | 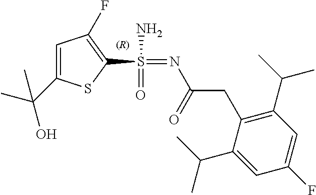 |
| 140b | 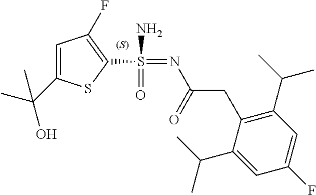 |
| 141 | 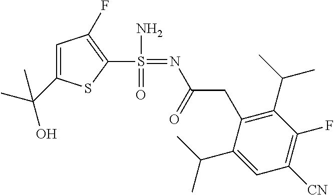 [SMILES: CC(C)c1cc(C#N)c(F)c(C(C)C)c1CC(=O)N=S(N)(=O)c1sc(C(C)(C)O)cc1F] |
| 141a | 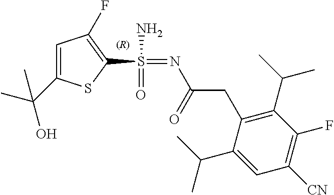 |
| 141b | 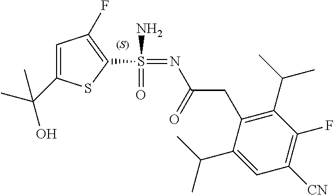 |
| 142 | 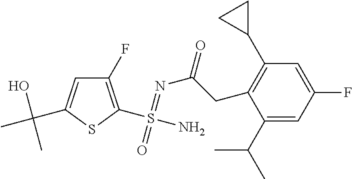 [SMILES: CC(C)c1cc(F)cc(C2CC2)c1CC(=O)N=S(N)(=O)c1sc(C(C)(C)O)cc1F] |
| 143 | 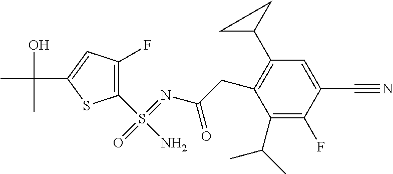 [SMILES: CC(C)c1c(F)c(C#N)cc(C2CC2)c1CC(=O)N=S(N)(=O)c1sc(C(C)(C)O)cc1F] |
| 144 | 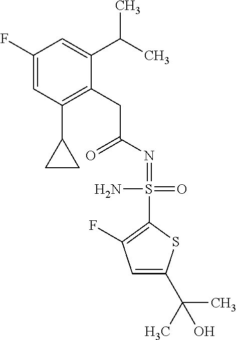 [SMILES: CC(C)c1cc(F)cc(C2CC2)c1CC(=O)N=S(N)(=O)c1sc(C(C)(C)O)cc1F] |
| 144a | 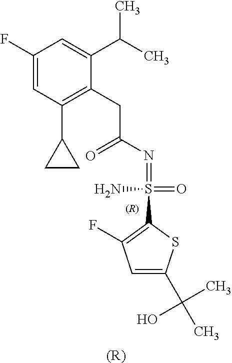 |
| 144b | 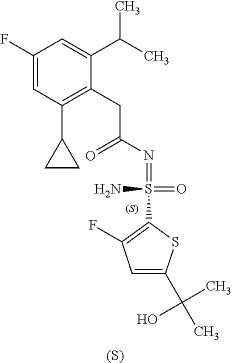 |
| 145 | 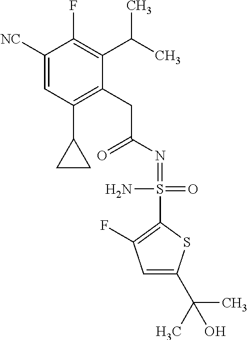 [SMILES: CC(C)c1c(F)c(C#N)cc(C2CC2)c1CC(=O)N=S(N)(=O)c1sc(C(C)(C)O)cc1F] |
| 145a | 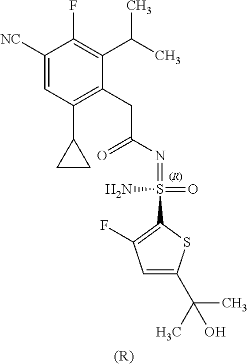 |
| 145b | 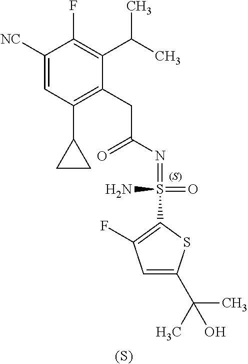 |
| 146 | 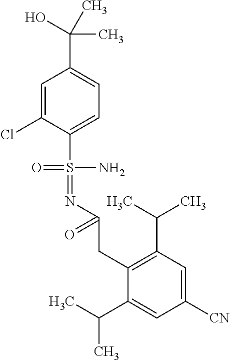 [SMILES: CC(C)c1cc(C#N)cc(C(C)C)c1CC(=O)N=S(N)(=O)c1ccc(C(C)(C)O)cc1Cl] |
| 147 | 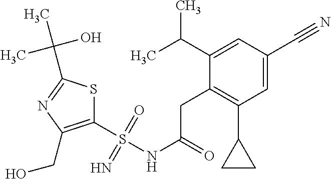 [SMILES: CC(C)c1cc(C#N)cc(C2CC2)c1CC(=O)NS(=N)(=O)c1sc(C(C)(C)O)nc1CO] |
| 147a | 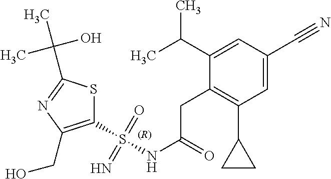 |
| 147b | 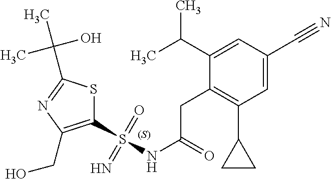 |
| 148 | 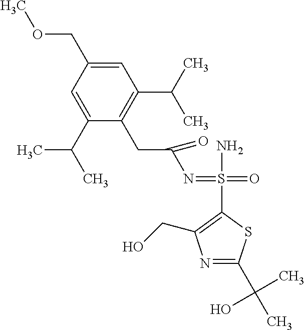 [SMILES: COCc1cc(C(C)C)c(CC(=O)N=S(N)(=O)c2sc(C(C)(C)O)nc2CO)c(C(C)C)c1] |
| 148a | |
| 148b | |
| 149a | |
| 149b | |
| 150 | 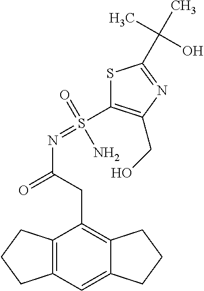 [SMILES: CC(C)(O)c1nc(CO)c(S(N)(=O)=NC(=O)Cc2c3c(cc4c2CCC4)CCC3)s1] |
| 150a | 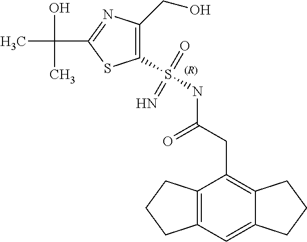 |
| 150b | 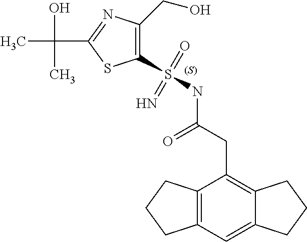 |
| 151 | |
| 152 | 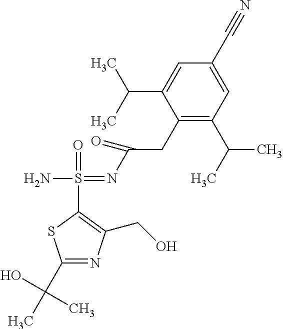 [SMILES: CC(C)c1cc(C#N)cc(C(C)C)c1CC(=O)N=S(N)(=O)c1sc(C(C)(C)O)nc1CO] |
| 152a | |
| 152b | |
| 153 | 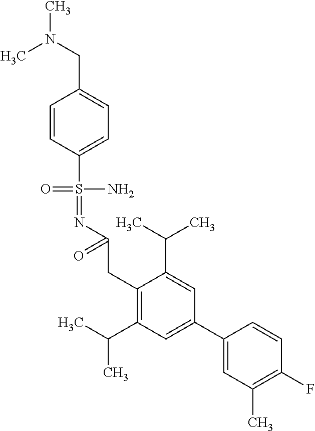 [SMILES: Cc1cc(-c2cc(C(C)C)c(CC(=O)N=S(N)(=O)c3ccc(CN(C)C)cc3)c(C(C)C)c2)ccc1F] |
| 154 | 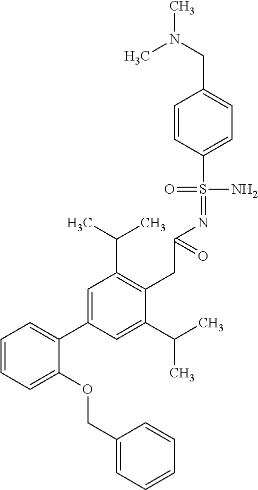 [SMILES: CC(C)c1cc(-c2ccccc2OCc2ccccc2)cc(C(C)C)c1CC(=O)N=S(N)(=O)c1ccc(CN(C)C)cc1] |
| 155 | 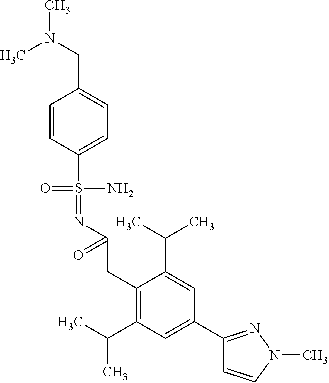 |
| 156 | 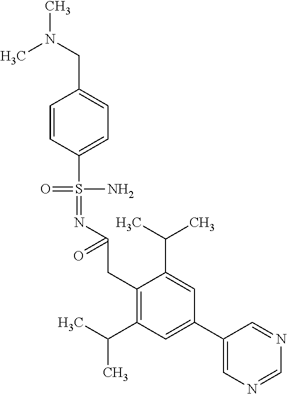 [SMILES: CC(C)c1cc(-c2cncnc2)cc(C(C)C)c1CC(=O)N=S(N)(=O)c1ccc(CN(C)C)cc1] |
| 157 | 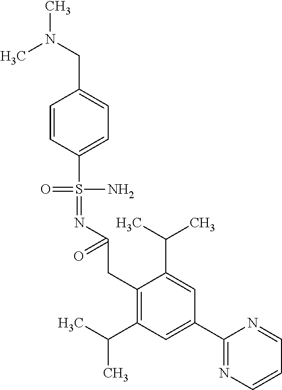 [SMILES: CC(C)c1cc(-c2ncccn2)cc(C(C)C)c1CC(=O)N=S(N)(=O)c1ccc(CN(C)C)cc1] |
| 158 | 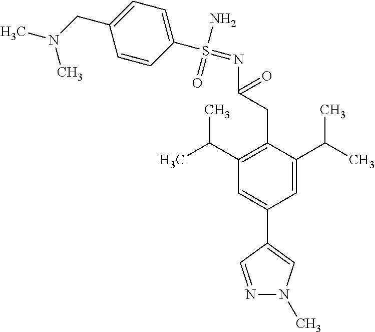 [SMILES: CC(C)c1cc(-c2cnn(C)c2)cc(C(C)C)c1CC(=O)N=S(N)(=O)c1ccc(CN(C)C)cc1] |
| 159 | 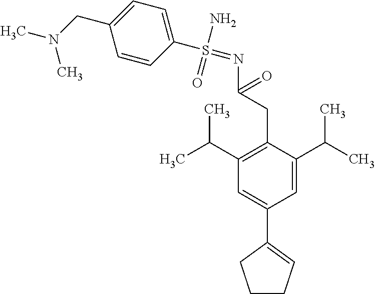 [SMILES: CC(C)c1cc(C2=CCCC2)cc(C(C)C)c1CC(=O)N=S(N)(=O)c1ccc(CN(C)C)cc1] |
| 160 | 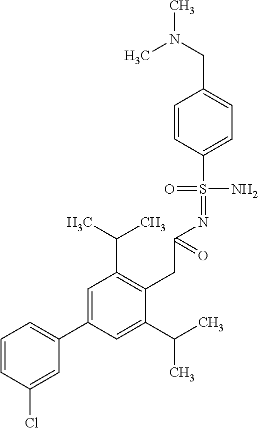 [SMILES: CC(C)c1cc(-c2cccc(Cl)c2)cc(C(C)C)c1CC(=O)N=S(N)(=O)c1ccc(CN(C)C)cc1] |
| 161 | 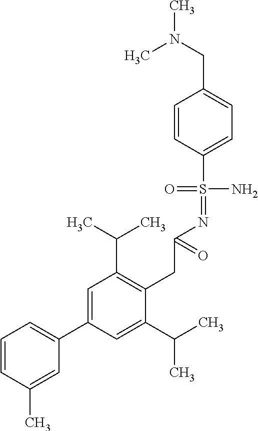 [SMILES: Cc1cccc(-c2cc(C(C)C)c(CC(=O)N=S(N)(=O)c3ccc(CN(C)C)cc3)c(C(C)C)c2)c1] |
| 162 | 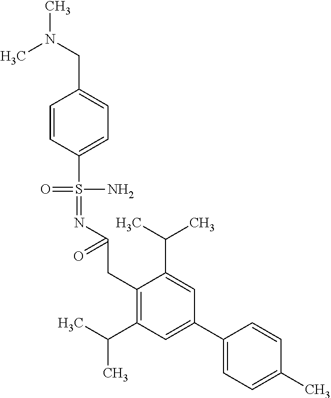 |
| 163 | 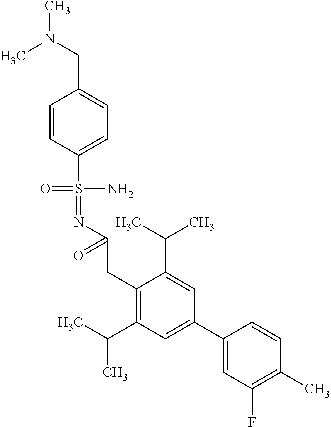 |
| 164 | 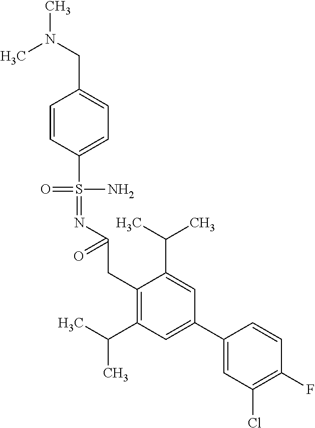 [SMILES: CC(C)c1cc(-c2ccc(F)c(Cl)c2)cc(C(C)C)c1CC(=O)N=S(N)(=O)c1ccc(CN(C)C)cc1] |
| 165 | 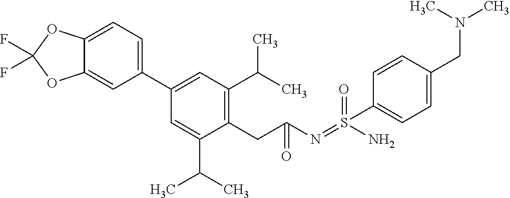 [SMILES: CC(C)c1cc(-c2ccc3c(c2)OC(F)(F)O3)cc(C(C)C)c1CC(=O)N=S(N)(=O)c1ccc(CN(C)C)cc1] |
| 166 | 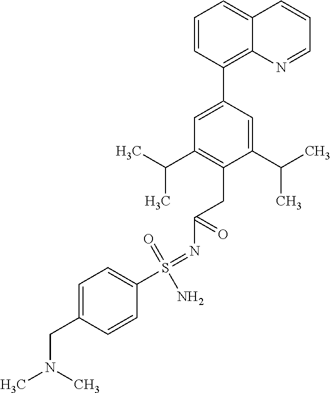 [SMILES: CC(C)c1cc(-c2cccc3cccnc23)cc(C(C)C)c1CC(=O)N=S(N)(=O)c1ccc(CN(C)C)cc1] |
| 167 | 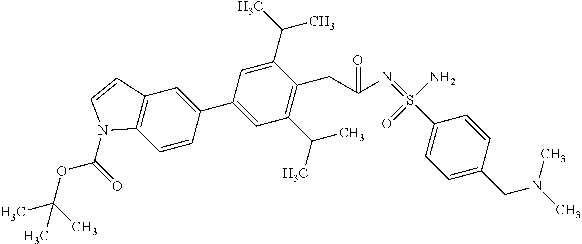 [SMILES: CC(C)c1cc(-c2ccc3c(ccn3C(=O)OC(C)(C)C)c2)cc(C(C)C)c1CC(=O)N=S(N)(=O)c1ccc(CN(C)C)cc1] |
| 168 | 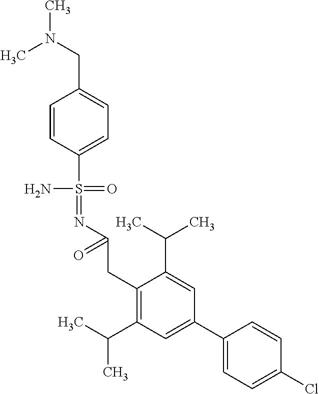 [SMILES: CC(C)c1cc(-c2ccc(Cl)cc2)cc(C(C)C)c1CC(=O)N=S(N)(=O)c1ccc(CN(C)C)cc1] |
| 169 | 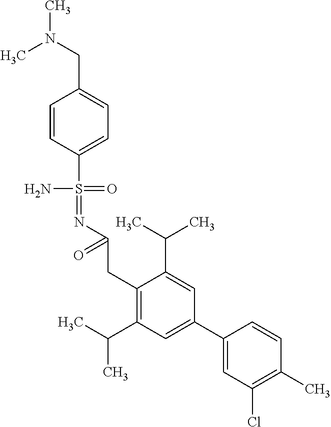 |
| 170 | 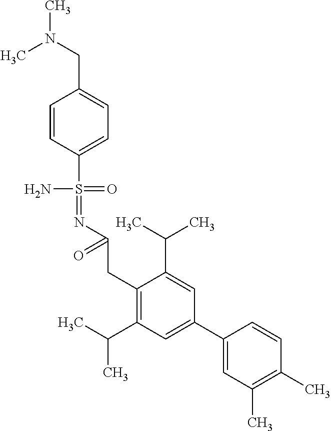 |
| 171 | 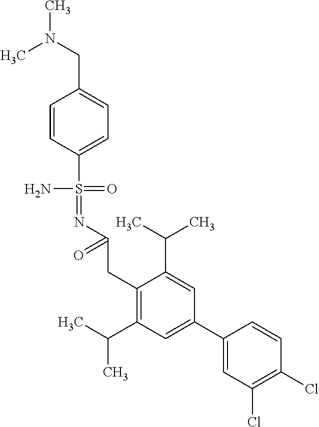 [SMILES: CC(C)c1cc(-c2ccc(Cl)c(Cl)c2)cc(C(C)C)c1CC(=O)N=S(N)(=O)c1ccc(CN(C)C)cc1] |
| 172 | 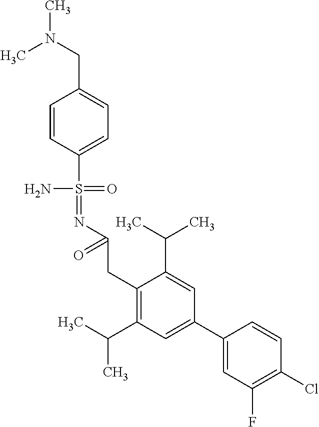 [SMILES: CC(C)c1cc(-c2ccc(Cl)c(F)c2)cc(C(C)C)c1CC(=O)N=S(N)(=O)c1ccc(CN(C)C)cc1] |
| 173 | 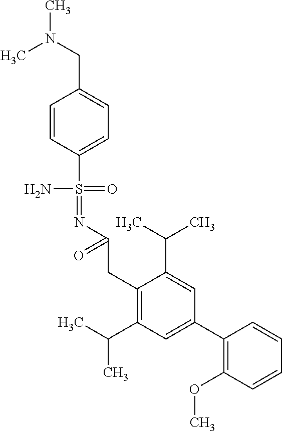 [SMILES: COc1ccccc1-c1cc(C(C)C)c(CC(=O)N=S(N)(=O)c2ccc(CN(C)C)cc2)c(C(C)C)c1] |
| 174 | 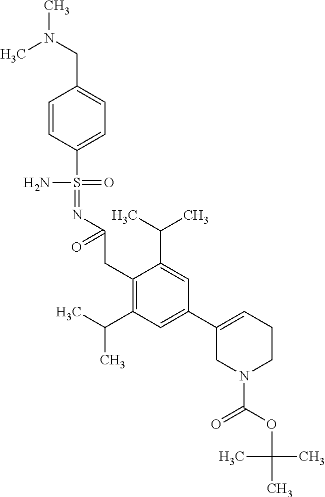 [SMILES: CC(C)c1cc(C2=CCCN(C(=O)OC(C)(C)C)C2)cc(C(C)C)c1CC(=O)N=S(N)(=O)c1ccc(CN(C)C)cc1] |
| 175 | 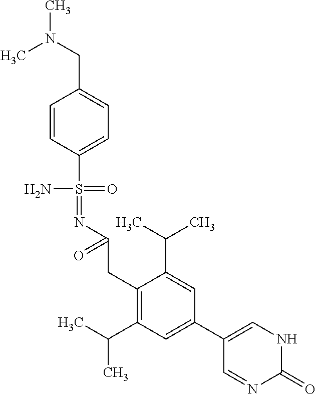 [SMILES: CC(C)c1cc(-c2cnc(=O)[nH]c2)cc(C(C)C)c1CC(=O)N=S(N)(=O)c1ccc(CN(C)C)cc1] |
| 176 | 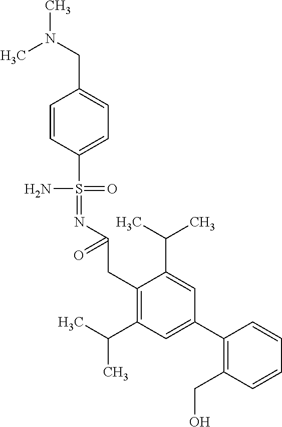 [SMILES: CC(C)c1cc(-c2ccccc2CO)cc(C(C)C)c1CC(=O)N=S(N)(=O)c1ccc(CN(C)C)cc1] |
| 177 | 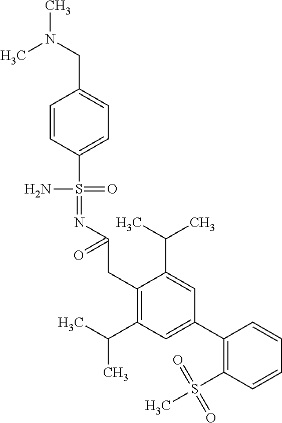 [SMILES: CC(C)c1cc(-c2ccccc2S(C)(=O)=O)cc(C(C)C)c1CC(=O)N=S(N)(=O)c1ccc(CN(C)C)cc1] |
| 178 | 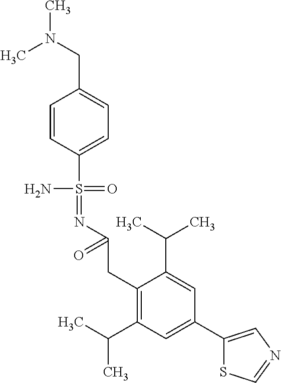 [SMILES: CC(C)c1cc(-c2cncs2)cc(C(C)C)c1CC(=O)N=S(N)(=O)c1ccc(CN(C)C)cc1] |
| 179 | 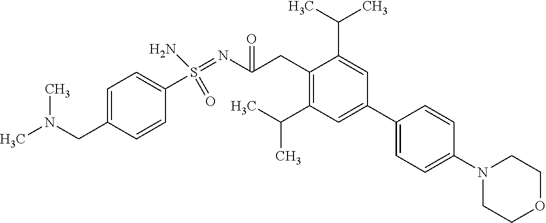 [SMILES: CC(C)c1cc(-c2ccc(N3CCOCC3)cc2)cc(C(C)C)c1CC(=O)N=S(N)(=O)c1ccc(CN(C)C)cc1] |
| 180 | 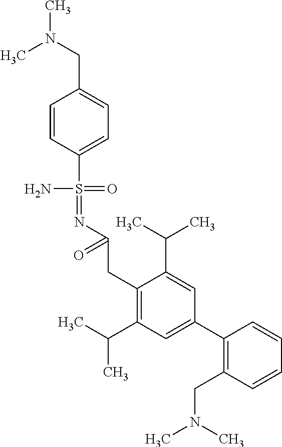 [SMILES: CC(C)c1cc(-c2ccccc2CN(C)C)cc(C(C)C)c1CC(=O)N=S(N)(=O)c1ccc(CN(C)C)cc1] |
| 181 | 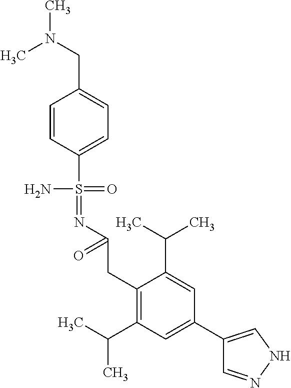 [SMILES: CC(C)c1cc(-c2cn[nH]c2)cc(C(C)C)c1CC(=O)N=S(N)(=O)c1ccc(CN(C)C)cc1] |
| 182 | 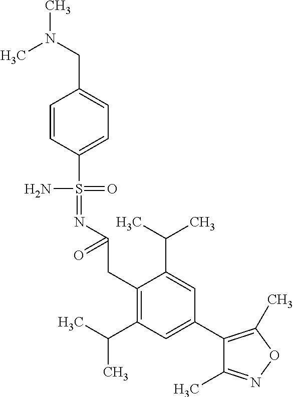 [SMILES: Cc1noc(C)c1-c1cc(C(C)C)c(CC(=O)N=S(N)(=O)c2ccc(CN(C)C)cc2)c(C(C)C)c1] |
| 183 | 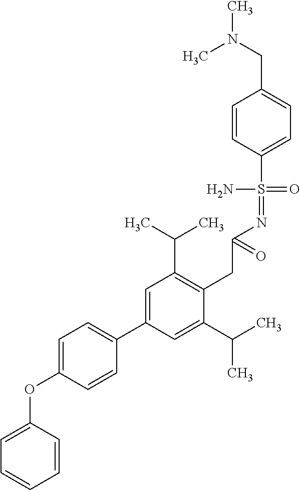 [SMILES: CC(C)c1cc(-c2ccc(Oc3ccccc3)cc2)cc(C(C)C)c1CC(=O)N=S(N)(=O)c1ccc(CN(C)C)cc1] |
| 184 | 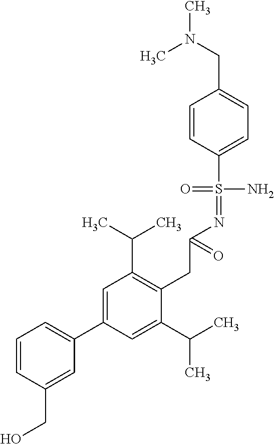 [SMILES: CC(C)c1cc(-c2cccc(CO)c2)cc(C(C)C)c1CC(=O)N=S(N)(=O)c1ccc(CN(C)C)cc1] |
| 185 | 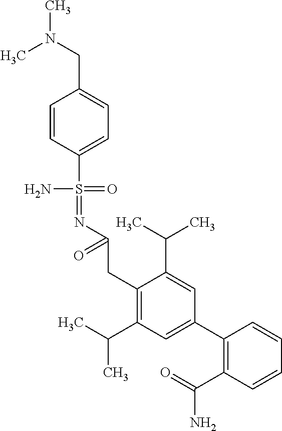 [SMILES: CC(C)c1cc(-c2ccccc2C(N)=O)cc(C(C)C)c1CC(=O)N=S(N)(=O)c1ccc(CN(C)C)cc1] |
| 186 | 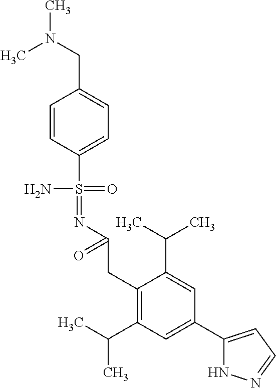 [SMILES: CC(C)c1cc(-c2ccn[nH]2)cc(C(C)C)c1CC(=O)N=S(N)(=O)c1ccc(CN(C)C)cc1] |
| 187 | 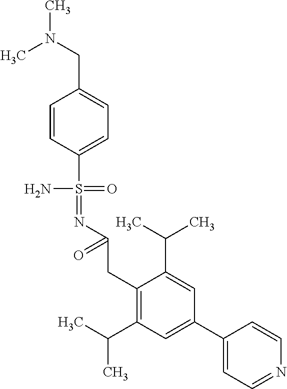 [SMILES: CC(C)c1cc(-c2ccncc2)cc(C(C)C)c1CC(=O)N=S(N)(=O)c1ccc(CN(C)C)cc1] |
| 188 | 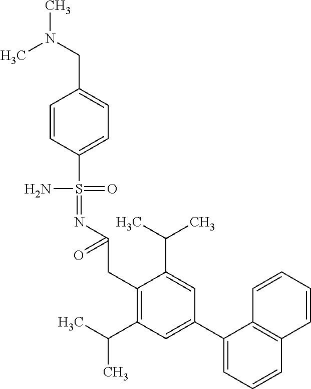 [SMILES: CC(C)c1cc(-c2cccc3ccccc23)cc(C(C)C)c1CC(=O)N=S(N)(=O)c1ccc(CN(C)C)cc1] |
| 189 | 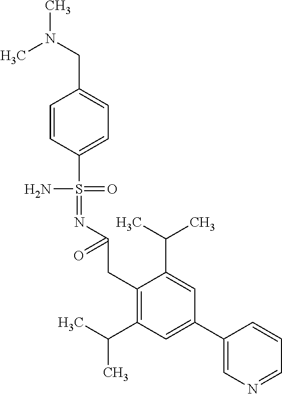 [SMILES: CC(C)c1cc(-c2cccnc2)cc(C(C)C)c1CC(=O)N=S(N)(=O)c1ccc(CN(C)C)cc1] |
| 190 | 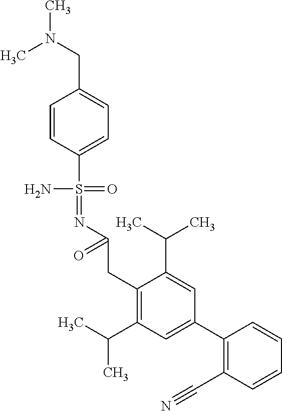 [SMILES: CC(C)c1cc(-c2ccccc2C#N)cc(C(C)C)c1CC(=O)N=S(N)(=O)c1ccc(CN(C)C)cc1] |
| 191 | 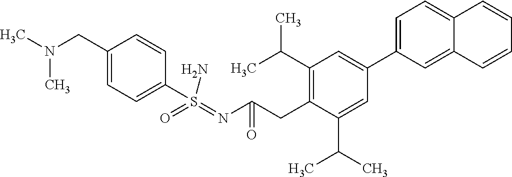 [SMILES: CC(C)c1cc(-c2ccc3ccccc3c2)cc(C(C)C)c1CC(=O)N=S(N)(=O)c1ccc(CN(C)C)cc1] |
| 192 | 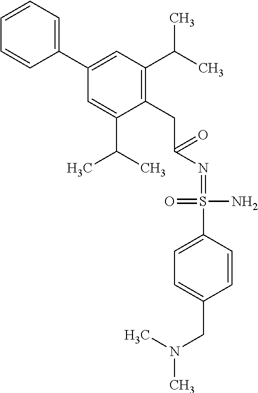 [SMILES: CC(C)c1cc(-c2ccccc2)cc(C(C)C)c1CC(=O)N=S(N)(=O)c1ccc(CN(C)C)cc1] |
| 193 | 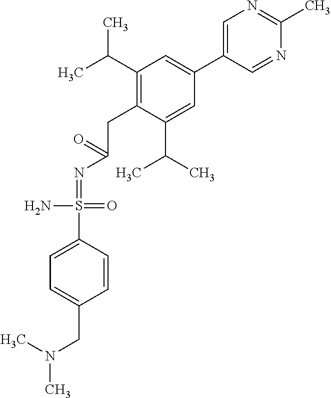 |
| 194 | 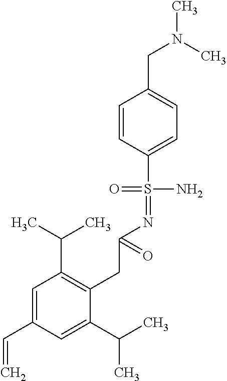 [SMILES: C=Cc1cc(C(C)C)c(CC(=O)N=S(N)(=O)c2ccc(CN(C)C)cc2)c(C(C)C)c1] |
| 195 | 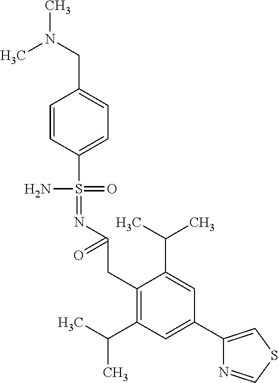 [SMILES: CC(C)c1cc(-c2cscn2)cc(C(C)C)c1CC(=O)N=S(N)(=O)c1ccc(CN(C)C)cc1] |
| 196 | 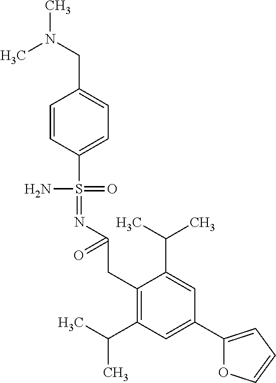 [SMILES: CC(C)c1cc(-c2ccco2)cc(C(C)C)c1CC(=O)N=S(N)(=O)c1ccc(CN(C)C)cc1] |
| 197 | 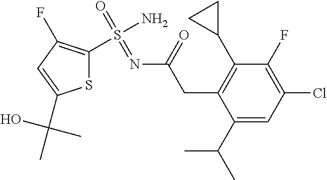 [SMILES: CC(C)c1cc(Cl)c(F)c(C2CC2)c1CC(=O)N=S(N)(=O)c1sc(C(C)(C)O)cc1F] |
| 197a | 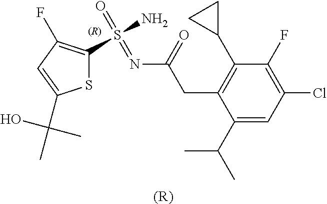 |
| 197b | 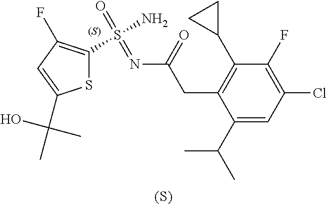 |
| 198 | 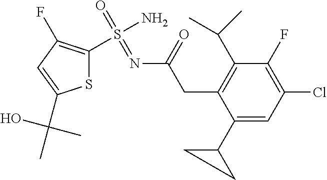 [SMILES: CC(C)c1c(F)c(Cl)cc(C2CC2)c1CC(=O)N=S(N)(=O)c1sc(C(C)(C)O)cc1F] |
| 198a | 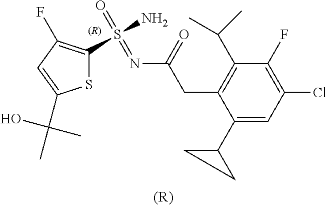 |
| 198b | 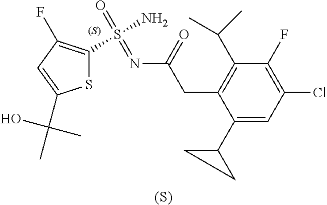 |

In another aspect, an NLRP3 antagonist is a compound selected from the group consisting of the compounds in Table 8 below and pharmaceutically acceptable salts thereof.

TABLE 8

| Com- | |
|---|---|
| pound | Stucture |
| 104a | 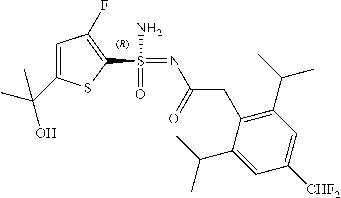 |
| 104b | 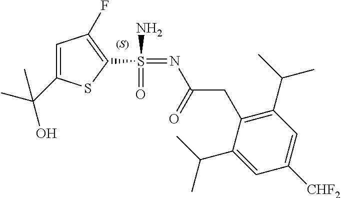 |
| 106a | 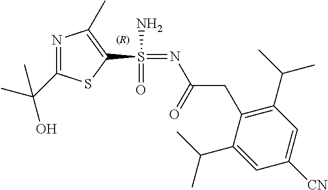 |
| 106b | 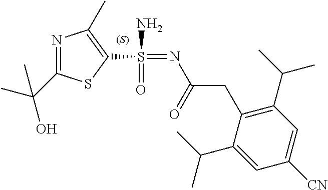 |
| 107a | 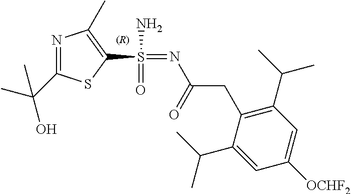 |
| 107b | 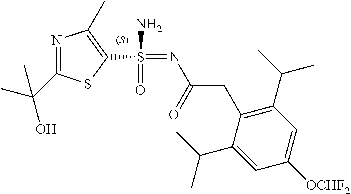 |
| 110a | 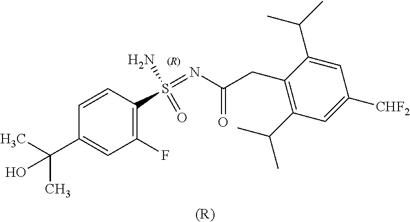 |
| 110b | 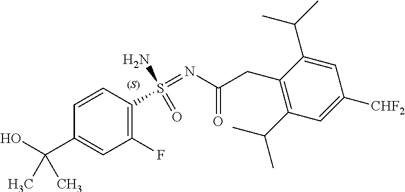 |
| 126a | 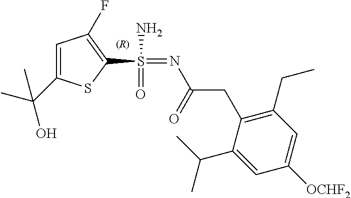 |
| 126b | 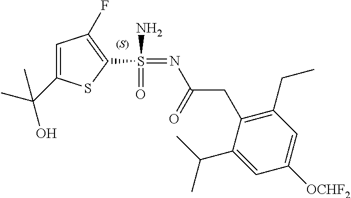 |
| 130a | 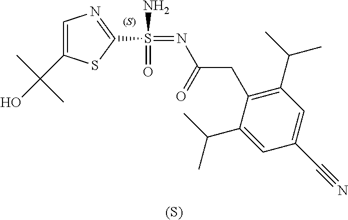 |
| 130b | 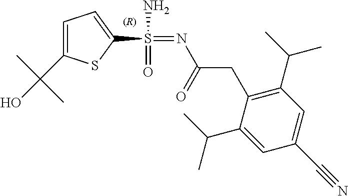 |
| 146 | 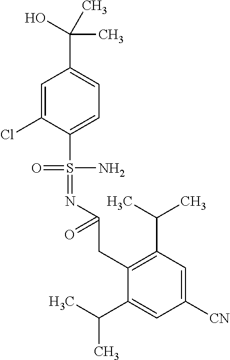 [SMILES: CC(C)c1cc(C#N)cc(C(C)C)c1CC(=O)N=S(N)(=O)c1ccc(C(C)(C)O)cc1Cl] |
| 147 | 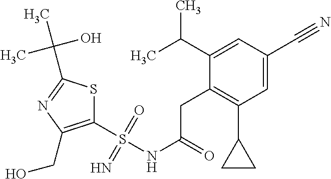 [SMILES: CC(C)c1cc(C#N)cc(C2CC2)c1CC(=O)NS(=N)(=O)c1sc(C(C)(C)O)nc1CO] |
| 148 | 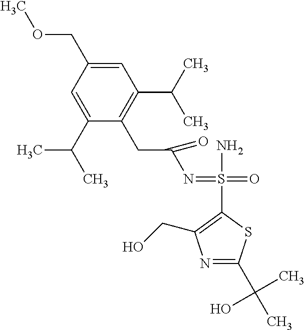 [SMILES: COCc1cc(C(C)C)c(CC(=O)N=S(N)(=O)c2sc(C(C)(C)O)nc2CO)c(C(C)C)c1] |
| 149a | 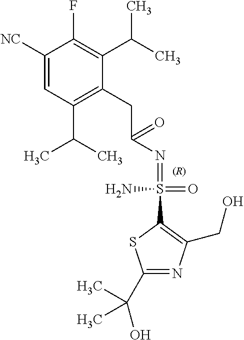 |
| 149b | 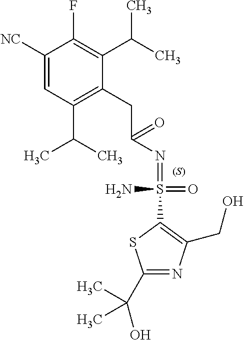 |
| 150 | 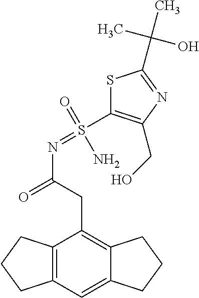 [SMILES: CC(C)(O)c1nc(CO)c(S(N)(=O)=NC(=O)Cc2c3c(cc4c2CCC4)CCC3)s1] |
| 151 | 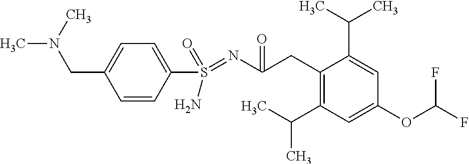 [SMILES: CC(C)c1cc(OC(F)F)cc(C(C)C)c1CC(=O)N=S(N)(=O)c1ccc(CN(C)C)cc1] |
| 152 | 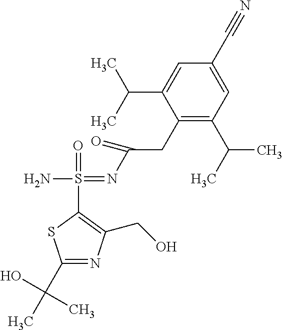 [SMILES: CC(C)c1cc(C#N)cc(C(C)C)c1CC(=O)N=S(N)(=O)c1sc(C(C)(C)O)nc1CO] |
| 152a | 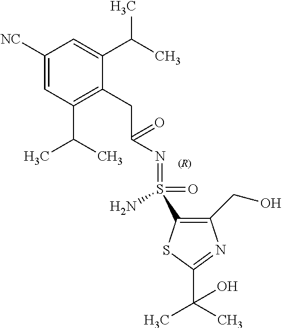 |
| 152b | 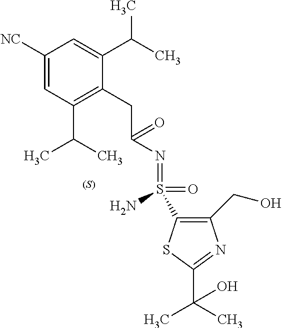 |

In some embodiments of the compound of Formula VIII, the sulfur in the moiety S(═O)(NR³)═N— has (S) stereochemistry.

In some embodiments of the compound of Formula VIII, the sulfur in the moiety S(═O)(NR³)═N— has (R) stereochemistry.

Compounds having Formula VIII, as well as methods of making and using the same, are further described in U.S. Provisional 62/573,935, filed on Oct. 18, 2017; and U.S. Provisional 62/536,352, filed on Jul. 24, 2017, each of which is incorporated herein by reference in its entirety.

In one aspect, an NLRP3 antagonist is a compound of Formula IX,

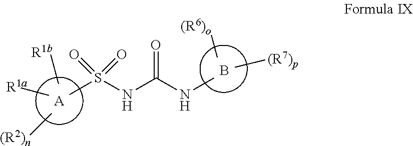

wherein

n=0 or 1;

o=1 or 2;

p=0, 1, 2, or 3;

wherein

A is a 5- to 10-membered heteroaryl or a C₆-C₁₀ aryl;

B is a 5- to 10-membered heteroaryl or a C₆-C₁₀ aryl;

wherein

R^{1a} is a C₁-C₆ alkyl or —SO₂NR¹¹R¹²;

wherein the C₁-C₆ alkyl is substituted with one or more hydroxy or —OSi(R¹³)₃; R^{1b} is a C₁-C₆ alkyl substituted with one or more hydroxy, —SO₂NR¹¹R¹², —SO₂R¹³, —CONR¹¹R¹², —OR¹¹, —CORD, —CO₂R¹³, —NR¹³CONR¹¹R¹², —CR¹¹R¹²CN, —NR¹¹SO₂R¹³, NR¹¹CONR¹², —CR¹¹R¹²NR¹¹R¹², and —NR¹¹COR¹²;

at least one R⁶ is ortho to the bond connecting the B ring to the NH(CO) group of Formula IX;

R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO (5- to 10-membered heteroaryl), CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), and OCO (3- to 7-membered heterocycloalkyl);
wherein each C₁-C₆ alkyl substituent and each C₁-C₆ alkoxy substituent of the R² C₃-C₇ cycloalkyl or of the R² 3-to 7-membered heterocycloalkyl is further optionally independently substituted with one to three hydroxy, halo, or oxo; wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3-to 7-membered heterocycloalkyl are optionally substituted with one or more substituents independently selected from halo, C₁-C₆ alkyl, and OC₁-C₆ alkyl;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, NR¹⁰, COOC₁-C₆ alkyl, C₆-C₁₀ aryl, and CONR⁸R⁹;

each of R⁴ and R⁵ is independently selected from hydrogen and C₁-C₆ alkyl; R¹⁰ is C₁-C₆ alkyl;

each of R⁸ and R⁹ at each occurrence is independently selected from hydrogen, C₁-C₆ alkyl, (C═NR¹³)NR¹¹R¹², S(O)₂C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;

R¹³ is C₁-C₆ alkyl, C₆-C₁₀ aryl, or 5- to 10-membered heteroaryl; and each of R¹¹ and R¹² at each occurrence is independently selected from hydrogen and C₁-C₆ alkyl optionally substituted with hydroxy;

or a pharmaceutically acceptable salt thereof.

In another aspect, an NLRP3 antagonist is a compound of Formula IX,

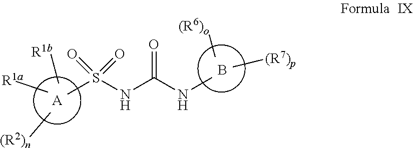

wherein

n=0 or 1;

o=1 or 2;

p=0, 1, 2, or 3;

wherein

A is a 5- to 10-membered heteroaryl or a C₆-C₁₀ aryl;

B is a 5- to 10-membered heteroaryl or a C₆-C₁₀ aryl;

wherein

R^{1a} is a C₁-C₆ alkyl or —SO₂NR¹¹R¹²;

wherein the C₁-C₆ alkyl is substituted with one or more hydroxy or —OSi(R¹³)₃; R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SP₂R¹³, —NR¹¹CONR¹¹R¹²—CR¹¹R¹²NR¹¹R¹², and —NR¹¹COR¹²;
at least one R⁶ is ortho to the bond connecting the B ring to the NH(CO) group of Formula IX;
R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO (5- to 10-membered heteroaryl), CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), and OCO (3- to 7-membered heterocycloalkyl);
   wherein each C₁-C₆ alkyl substituent and each C₁-C₆ alkoxy substituent of the R² C₃-C₇ cycloalkyl or of the R² 3-to 7-membered heterocycloalkyl is further optionally independently substituted with one to three hydroxy, halo, or oxo; wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3-to 7-membered heterocycloalkyl are optionally substituted with one or more substituents independently selected from halo, C₁-C₆ alkyl, and OC₁-C₆ alkyl;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;
   or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

each of R⁴ and R⁵ is independently selected from hydrogen and C₁-C₆ alkyl; R¹⁰ is C₁-C₆ alkyl;

each of R⁸ and R⁹ at each occurrence is independently selected from hydrogen, C₁-C₆ alkyl, (C═NR¹³)NR¹¹R¹², S(O)₂C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;

R¹³ is C₁-C₆ alkyl, C₆-C₁₀ aryl, or 5- to 10-membered heteroaryl; and each of R¹¹ and 10² at each occurrence is independently selected from hydrogen and C₁-C₆ alkyl optionally substituted with hydroxy;

with the proviso that the compound of Formula IX is not a compound selected from the group consisting of:

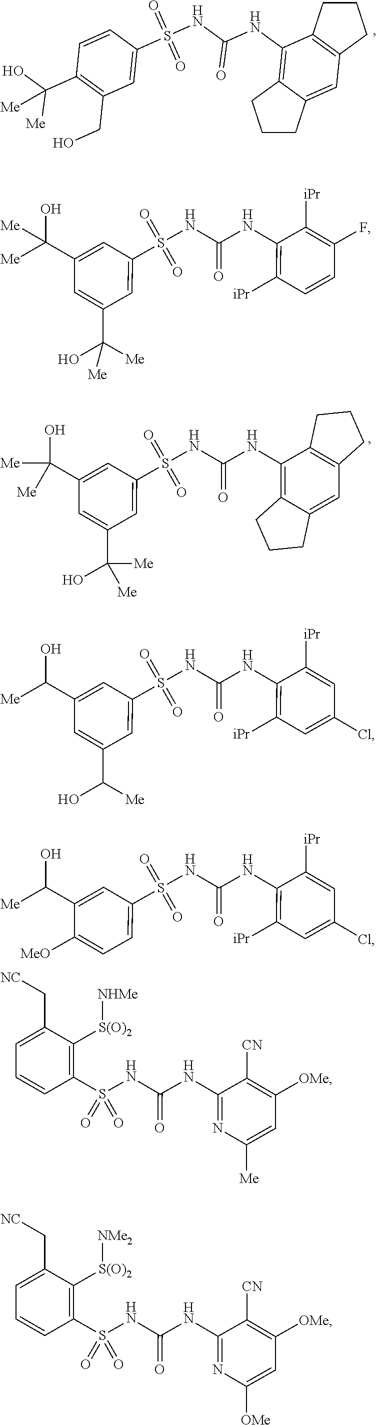

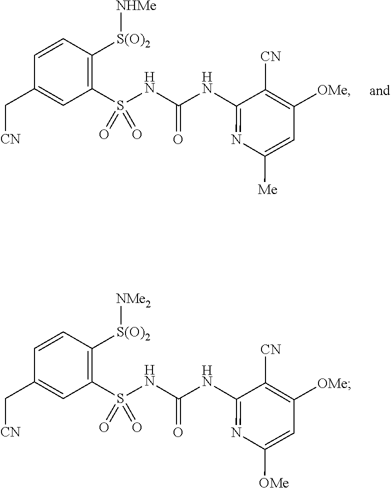

or a pharmaceutically acceptable salt thereof.

In another aspect, an NLRP3 antagonist is a compound of Formula IX

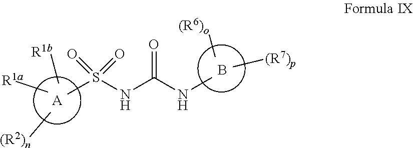

wherein the compound of Formula IX is selected from

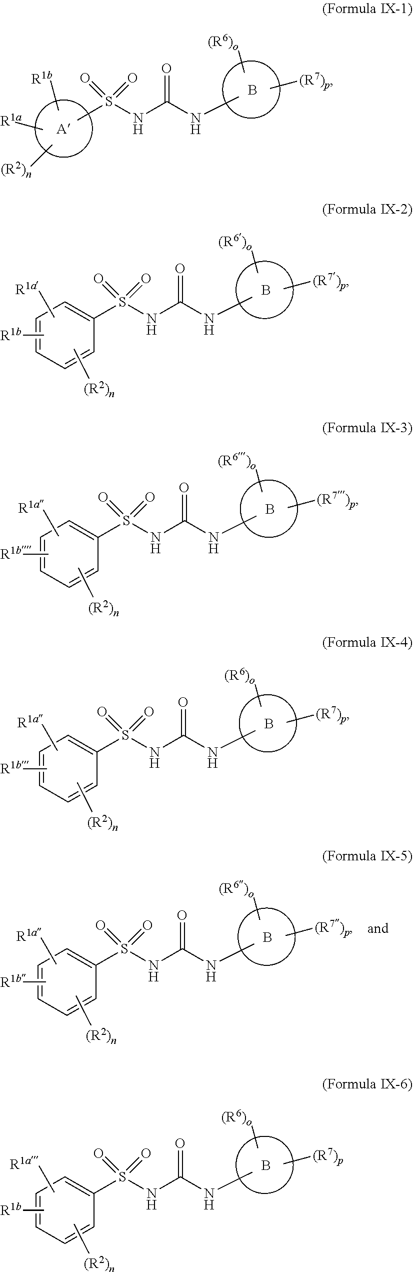

wherein

n=0 or 1;

o=1 or 2;

p=0, 1, 2, or 3;

wherein

A′ is a 5- to 10-membered heteroaryl;

B is a 5- to 10-membered heteroaryl or a C₆-C₁₀ aryl;

wherein

R^{1a} is a C₁-C₆ alkyl or —SO₂NR¹¹R¹²;

wherein the C₁-C₆ alkyl is substituted with one or more hydroxy or —OSi(R¹³)₃;

R^{1a′} is —SO₂NR¹¹R¹²;

R^{1a″} is a C₁-C₆ alkyl;

wherein the C₁-C₆ alkyl is substituted with one or more hydroxy;

R^{1b} is a C₁-C₆ alkyl;

wherein the C₁-C₆ alkyl is substituted with one or more —OSi(R¹³)₃;

R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R₁₃, —NR¹¹CONR¹¹R¹², —CR¹¹R¹²NR¹¹R¹², and —NR¹¹COR¹²;

R^{1b″} is —OR¹¹;

R^{1b′″} is a —SO₂NR¹¹R¹², —SO₂R¹³, —COR¹³; —CO₂R¹³, —NR¹³CONR¹¹R¹²; —CR¹¹R¹²CN, —NR¹¹SO₂R¹³, —CR¹¹R¹²NR¹¹R¹², and —NR¹¹COR¹²;

R^{1b″″} is a C₁-C₆ alkyl substituted with one or more hydroxy;
at least one R⁶ is ortho to the bond connecting the B ring to the NH(CO) group of Formula IX-1 and Formula IX-4;
at least one R^{6′} is ortho to the bond connecting the B ring to the NH(CO) group of Formula IX-2;

at least one R^{6″} is ortho to the bond connecting the B ring to the NH(CO) group of Formula IX-5;

at least one R^{6′″} is ortho to the bond connecting the B ring to the NH(CO) group of Formula IX-3;

R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO (5- to 10-membered heteroaryl), CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), and OCO (3- to 7-membered heterocycloalkyl);
   wherein each C₁-C₆ alkyl substituent and each C₁-C₆ alkoxy substituent of the R² C₃-C₇ cycloalkyl or of the R² 3-to 7-membered heterocycloalkyl is further optionally independently substituted with one to three hydroxy, halo, or oxo; wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3-to 7-membered heterocycloalkyl are optionally substituted with one or more substituents independently selected from halo, C₁-C₆ alkyl, and OC₁-C₆ alkyl;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

R^{6′} and R^{7′} are each independently selected from C₁-C₆ alkyl, C₁-C₆ haloalkyl, C₂-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
wherein R^{6′} and R^{7′} are each optionally substituted with one or more substituents independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R^{6′} or R⁷ is substituted with is optionally substituted with one or more hydroxyl, halo, C₆-C₁₀ aryl or NR⁸R⁹, or wherein R^{6′} or R⁷ is optionally fused to a five-to-seven-membered carbocyclic ring or heterocyclic ring containing one or two heteroatoms independently selected from oxygen, sulfur and nitrogen;
   wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;
or at least one pair of R^{6′} and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR₁₀, COOC₁-C₆ alkyl, C₆-C₁₀ aryl, and CONR⁸R⁹;

R^{6″} and R^{7″} are each independently selected from C₁-C₆ alkyl, C₁-C₆ haloalkyl, C₁-C₆ alkoxy, C₁-C₆ haloalkoxy, F, Br, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
wherein R⁶″ and R⁷″ are each optionally substituted with one or more substituents independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R^{6″} or R^{7″} is substituted with is optionally substituted with one or more hydroxyl, halo, C₆-C₁₀ aryl or NR⁸R⁹, or wherein R⁶″ or R⁷″ is optionally fused to a five-to-seven-membered carbocyclic ring or heterocyclic ring containing one or two heteroatoms independently selected from oxygen, sulfur and nitrogen;
   wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;
or at least one pair of R^{6″} and R^{7″} on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

R^{6′″} and R^{7′″} are each independently selected from C₁-C₆ alkyl, C₁-C₆ haloalkyl, C₁-C₆ alkoxy, C₁-C₆ haloalkoxy, Br, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,
wherein R^{6′″} and R^{7′″} are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R^{6′″} or R^{7′″} is substituted with is optionally substituted with one or more hydroxyl, halo, C₆-C₁₀ aryl or NR⁸R⁹, or wherein R^{6′″} or R^{7′″} is optionally fused to a five-to-seven-membered carbocyclic ring or heterocyclic ring containing one or two heteroatoms independently selected from oxygen, sulfur and nitrogen;
   wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;
or at least one pair of R^{6′″} and R^{7′″} on adjacent atoms, taken together with the atoms connecting them, independently form at least one C₄, C₆, C₇, or C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

each of R⁴ and R⁵ is independently selected from hydrogen and C₁-C₆ alkyl;

R¹⁰ is C₁-C₆ alkyl;

each of R⁸ and R⁹ at each occurrence is independently selected from hydrogen, C₁-C₆ alkyl, (C═NR¹³)NR¹¹R¹², S(O)₂C₁-C₆ alkyl, S(O₂)NR¹¹R¹², COR¹³, CO₂R¹³ and CONR¹¹R¹², wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl optionally substituted with hydroxy;
or a pharmaceutically acceptable salt thereof.

In some embodiments of the compound of Formula IX, the compound of Formula IX is

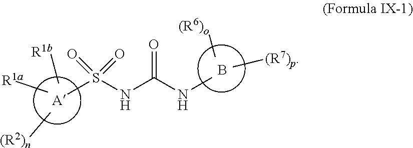

In some embodiments of the compound of Formula IX, the compound of Formula IX is

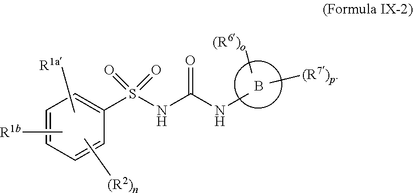

In some embodiments of the compound of Formula IX, the compound of Formula IX is

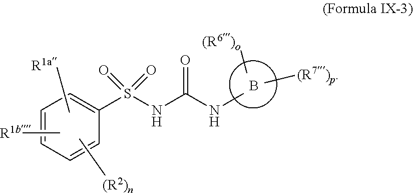

In some embodiments of the compound of Formula IX, the compound of Formula IX is

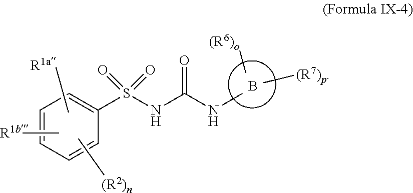

In some embodiments of the compound of Formula IX, the compound of Formula IX is

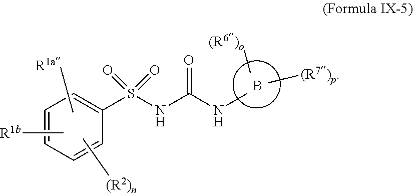

In some embodiments of the compound of Formula IX, the compound of Formula IX is

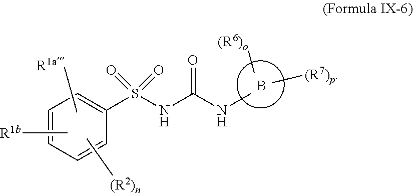

In some embodiments of the compound of Formula IX, A is a 5- to 6-membered heteroaryl containing 1 sulfur ring member.

In some embodiments of the compound of Formula IX, A is thiazolyl.

In some embodiments of the compound of Formula IX, A is pyrazolyl.

In some embodiments of the compound of Formula IX, n=0.

In some embodiments of the compound of Formula IX, the substituted ring A is

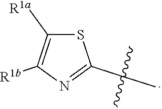

In some embodiments of the compound of Formula IX, the substituted ring A is

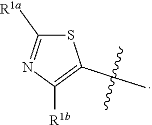

In some embodiments of the compound of Formula IX, the substituted ring A is

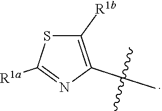

In some embodiments of the compound of Formula IX, the substituted ring A is

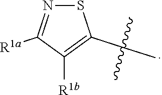

In some embodiments of the compound of Formula IX, the substituted ring A is

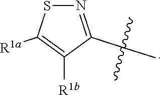

In some embodiments of the compound of Formula IX, the substituted ring A is

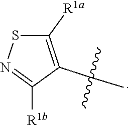

In some embodiments of the compound of Formula IX, n=1.

In some embodiments of the compound of Formula IX, the substituted ring A is

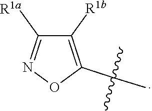

In some embodiments of the compound of Formula IX, the substituted ring A is

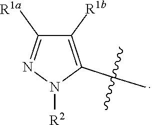

In some embodiments of the compound of Formula IX, the substituted ring A is

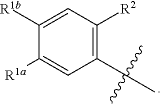

In some embodiments of the compound of Formula IX, wherein the substituted ring

A is

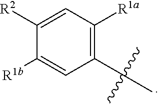

In some embodiments of the compound of Formula IX, the substituted ring A is

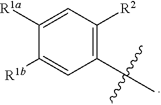

In some embodiments of the compound of Formula IX, the substituted ring A is

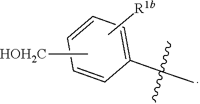

In some embodiments of the compound of Formula IX, the substituted ring A is

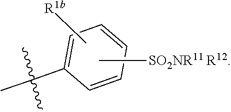

In some embodiments of the compound of Formula IX, the substituted ring A is

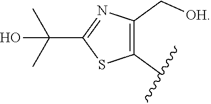 [SMILES: *c1sc(C(C)(C)O)nc1CO]

In some embodiments of the compound of Formula IX, the substituted ring A is

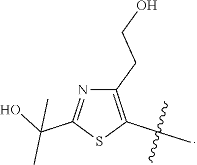 [SMILES: *c1sc(C(C)(C)O)nc1CCO]

In some embodiments of the compound of Formula IX, the substituted ring A is

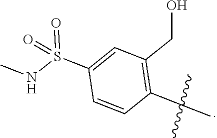 [SMILES: *c1ccc(S(=O)(=O)NC)cc1CO]

In some embodiments of the compound of Formula IX, the substituted ring A is

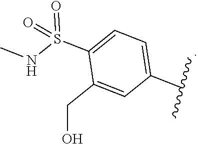 [SMILES: *c1ccc(S(=O)(=O)NC)c(CO)c1]

In some embodiments of the compound of Formula IX, R^{1a} is C₁-C₆ alkyl substituted with one or more hydroxy.

In some embodiments of the compound of Formula IX, R^{1a} is C₁-C₆ alkyl substituted with one or more —OSi(R¹³)₃.

In some embodiments of the compound of Formula IX, R^{1a} is C₁-C₆ alkyl substituted with one or more —SO₂NR¹¹R¹².

In some embodiments of the compound of Formula IX, R^{1b} is independently selected from the group consisting of C₁-C₆ alkyl substituted with one or more hydroxy, —SO₂NR¹¹R¹², —SO₂R¹³, —CONR¹¹R¹², —OR¹¹, —COR¹³; —NR¹³CONR¹¹R¹²; —CR¹¹R¹²CN, —NR¹¹SO₂R¹³, —NR¹¹CONR¹¹R¹², and —NR¹¹COR¹².

In certain embodiments of the compound of Formula IX, R^{ib} is independently selected from the group consisting of —SO₂NR¹¹R¹², —SO₂R¹³, —CONR¹¹R¹², —COR¹³, —CO₂R¹³, —NR¹³CONR¹¹R¹²; and —CR¹¹R¹²CN.

In certain embodiments of the compound of Formula IX, R^{1b} is —SO₂NHMe, SO₂NHCH₂CH₂OH, SO₂Me, CONHMe, or OMe.

In certain embodiments of the compound of Formula IX, R^{1b} is —SO₂NHMe or OMe.

In some embodiments of the compound of Formula IX, R² is independently selected from the group consisting of hydroxymethyl, C₂ alkyl substituted with hydroxy, C₃ alkyl substituted with hydroxy, C₄ alkyl substituted with hydroxy, C₅ alkyl substituted with hydroxy, and C₆ alkyl substituted with hydroxy.

In certain embodiments of the compound of Formula IX, R² is selected from the group consisting of hydroxymethyl, 1-hydroxyethyl, 2-hydroxyethyl, 2-hydroxy-2-propyl, 3-hydroxy-2-propyl, 1-hydroxy-1-propyl, 2-hydroxy-1-propyl, 3-hydroxy-1-propyl, 4-hydroxy-1-butyl, 5-hydroxy-1-pentyl, and 6-hydroxy-1-hexyl.

In certain embodiments of the compound of Formula IX, R² is selected from the group consisting of hydroxymethyl, 1-hydroxyethyl, 2-hydroxyethyl, 2-hydroxy-2-propyl, 3-hydroxy-2-propyl, 1-hydroxy-1-propyl, 2-hydroxy-1-propyl, 3-hydroxy-1-propyl, 4-hydroxy-1-butyl, and 6-hydroxy-1-hexyl.

In some embodiments of the compound of Formula IX, R² is selected from the group consisting of C₁-C₆ alkyl optionally substituted with one or more hydroxy, halo, oxo, or C₁-C₆ alkoxy; C₃-C₇ cycloalkyl optionally substituted with one or more hydroxy, halo, oxo, C₁-C₆ alkoxy, or C₁-C₆ alkyl wherein the C₁-C₆ alkoxy or C₁-C₆ alkyl is further optionally substituted with one to three hydroxy, halo, or oxo; 3- to 7-membered heterocycloalkyl optionally substituted with one or more hydroxy, halo, oxo, or C₁-C₆ alkyl, wherein the C₁-C₆ alkoxy or C₁-C₆ alkyl is further optionally substituted with one to three hydroxy, halo, or oxo; C₁-C₆ haloalkyl; C₁-C₆ alkoxy; C₁-C₆ haloalkoxy; halo; CN; CO—C₁-C₆ alkyl; CO—C₆-C₁₀ aryl; CO (5- to 10-membered heteroaryl); CO₂C₁-C₆ alkyl; CO₂C₃-C₈ cycloalkyl; OCOC₁-C₆ alkyl; OCOC₆-C₁₀ aryl; OCO (5- to 10-membered heteroaryl); OCO (3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; S(O₂)NR¹¹R¹², S(O)C₁-C₆ alkyl; and S(O₂)C₁-C₆ alkyl.

In certain embodiments of the compound of Formula IX, R² is selected from the group consisting of fluoro; chloro; cyano; methyl; methoxy; ethoxy; isopropyl; 1-hydroxy-2-methylpropan-2-yl; 2-hydroxy-2-propyl; hydroxymethyl; 1-hydroxy ethyl; 2-hydroxyethyl; 1-hydroxy-2-propyl; 1-hydroxy-1-cyclopropyl; COCH₃; COPh; 2-methoxy-2-propyl; phenyl; S(O₂)CH₃, and S(O₂)NR¹¹R¹².

In some embodiments of the compound of Formula IX, B is phenyl substituted with 1 or 2 R⁶ and optionally substituted with 1, 2, or 3 R⁷.

In certain of the forgoing embodiments of Formula IX (when B is phenyl substituted with 1 or 2 R⁶ and optionally substituted with 1, 2, or 3 R⁷), o=2 and p=0.

In certain of the forgoing embodiments of Formula IX (when B is phenyl; o=2; and p=0), the optionally substituted ring B is

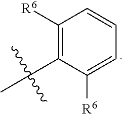

In certain of the forgoing embodiments of Formula IX (when the optionally substituted ring B is

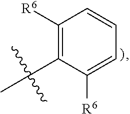

each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl.
   As non-limiting example of the above, each R⁶ is independently selected from the group consisting of: C₁-C₆ alkyl, C₃-C₇ cycloalkyl, C₁-C₆ haloalkyl, C₁-C₆ alkoxy, and C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.

In certain embodiments of Formula IX (when B is phenyl substituted with 1 or 2 R⁶ and optionally substituted with 1, 2, or 3 R⁷), o=1 and p=1.

In certain embodiments of Formula IX when B is phenyl substituted with 1 or 2 R⁶ and optionally substituted with 1, 2, or 3 R⁷, o=2 and p=1.
In certain of the foregoing embodiments of Formula IX (when B is phenyl; o=2; and p=1), the optionally substituted ring B is

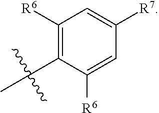

In certain of the foregoing embodiments of Formula IX (when the optionally substituted ring B is

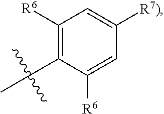

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
   wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

In certain embodiments of Formula IX (when B is phenyl substituted with 1 or 2 R⁶ and optionally substituted with 1, 2, or 3 R⁷), o=2 and p=2.

In certain of the foregoing embodiments of Formula IX (when B is phenyl; o=2; and p=2), the optionally substituted ring B is

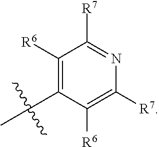

In certain of the foregoing embodiments of Formula IX (the optionally substituted ring B is

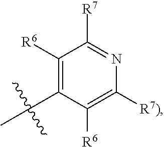

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
   wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain embodiments of Formula IX (when B is phenyl; o=2; and p=2), the optionally substituted ring B is

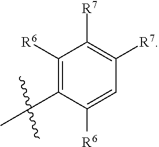

In certain of the foregoing embodiments of Formula IX (when the optionally substituted ring B is

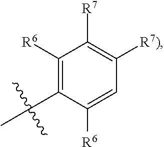

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,
wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
   wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
   or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain embodiments of Formula IX (when B is phenyl substituted with 1 or 2 R⁶ and optionally substituted with 1, 2, or 3 R⁷), o=2 and p=3.

In certain of the foregoing embodiments of Formula IX (when B is phenyl; o=2; and p=3), the optionally substituted ring B is

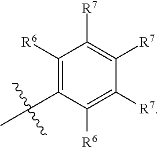

As non-limiting example of the above, the optionally substituted ring B can be

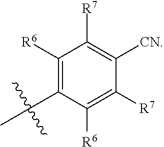

In some embodiments of the compound of Formula IX, the optionally substituted ring B is

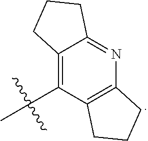 [SMILES: *c1c2c(nc3c1CCC3)CCC2]

In certain embodiments of Formula IX (when the optionally substituted ring B is

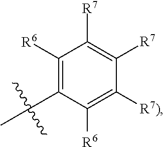

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments of the compound of Formula IX, two pairs of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹. In some embodiments of the compound of Formula IX, each R⁶ is independently selected from CN, C₁-C₆ alkyl, 5- to 10-membered heteroaryl, and 3- to 7-membered heterocycloalkyl;
wherein the C₁-C₆ alkyl is optionally substituted with one or more substituents each independently selected from hydroxyl or C₁-C₆ alkoxy.
   In some embodiments of the compound of Formula IX, each R⁷ is independently selected from CN, C₁-C₆ alkyl, 5- to 10-membered heteroaryl, and 3- to 7-membered heterocycloalkyl;
wherein the C₁-C₆ alkyl is optionally substituted with one or more substituents each independently selected from hydroxyl or C₁-C₆ alkoxy.

In some embodiments of the compound of Formula IX, R³ is hydrogen.

In another aspect, an NLRP3 antagonist is a compound selected from the group consisting of the compounds in Table 9 below and pharmaceutically acceptable salts thereof.

| Com- | |
|---|---|
| pound | Structure |
| 101 | 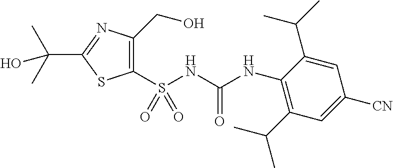 [SMILES: CC(C)c1cc(C#N)cc(C(C)C)c1NC(=O)NS(=O)(=O)c1sc(C(C)(C)O)nc1CO] |
| 102 | 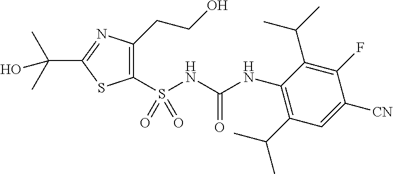 [SMILES: CC(C)c1cc(C#N)c(F)c(C(C)C)c1NC(=O)NS(=O)(=O)c1sc(C(C)(C)O)nc1CCO] |
| 103 | 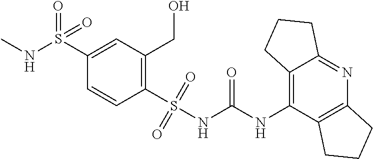 [SMILES: CNS(=O)(=O)c1ccc(S(=O)(=O)NC(=O)Nc2c3c(nc4c2CCC4)CCC3)c(CO)c1] |
| 104 | 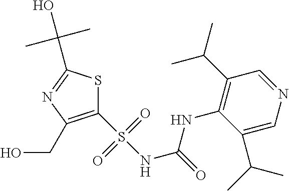 [SMILES: CC(C)c1cncc(C(C)C)c1NC(=O)NS(=O)(=O)c1sc(C(C)(C)O)nc1CO] |
| 105 | 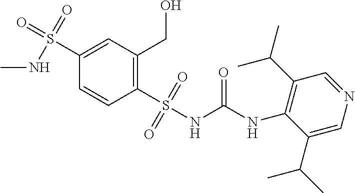 [SMILES: CNS(=O)(=O)c1ccc(S(=O)(=O)NC(=O)Nc2c(C(C)C)cncc2C(C)C)c(CO)c1] |
| 106 | 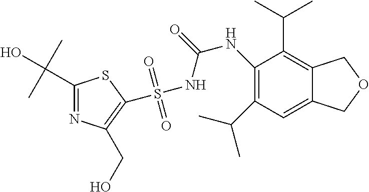 [SMILES: CC(C)c1cc2c(c(C(C)C)c1NC(=O)NS(=O)(=O)c1sc(C(C)(C)O)nc1CO)COC2] |
| 107 | 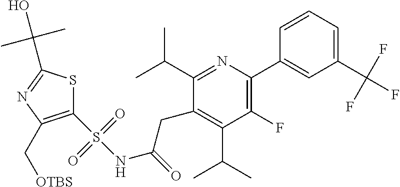 [SMILES: CC(C)c1nc(-c2cccc(C(F)(F)F)c2)c(F)c(C(C)C)c1CC(=O)NS(=O)(=O)c1sc(C(C)(C)O)nc1CO[Si](C)(C)C(C)(C)C] |
| 108 | 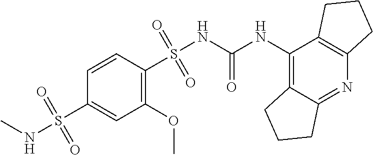 [SMILES: CNS(=O)(=O)c1ccc(S(=O)(=O)NC(=O)Nc2c3c(nc4c2CCC4)CCC3)c(OC)c1] |
| 109 | 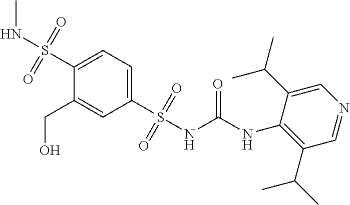 [SMILES: CNS(=O)(=O)c1ccc(S(=O)(=O)NC(=O)Nc2c(C(C)C)cncc2C(C)C)cc1CO] |
| 110 | 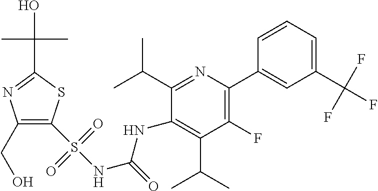 [SMILES: CC(C)c1nc(-c2cccc(C(F)(F)F)c2)c(F)c(C(C)C)c1NC(=O)NS(=O)(=O)c1sc(C(C)(C)O)nc1CO] |
| 111 | 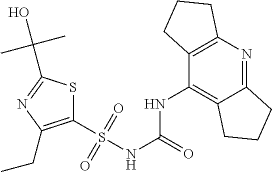 [SMILES: CCc1nc(C(C)(C)O)sc1S(=O)(=O)NC(=O)Nc1c2c(nc3c1CCC3)CCC2] |
| 112 | 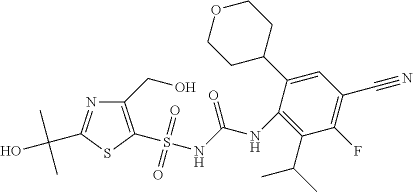 [SMILES: CC(C)c1c(F)c(C#N)cc(C2CCOCC2)c1NC(=O)NS(=O)(=O)c1sc(C(C)(C)O)nc1CO] |
| 113 | 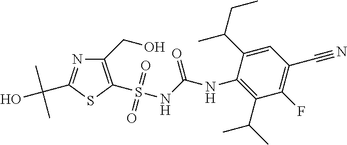 [SMILES: CCC(C)c1cc(C#N)c(F)c(C(C)C)c1NC(=O)NS(=O)(=O)c1sc(C(C)(C)O)nc1CO] |
| 114 | 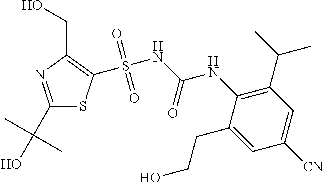 [SMILES: CC(C)c1cc(C#N)cc(CCO)c1NC(=O)NS(=O)(=O)c1sc(C(C)(C)O)nc1CO] |
| 115 | 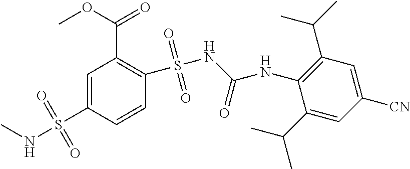 [SMILES: CNS(=O)(=O)c1ccc(S(=O)(=O)NC(=O)Nc2c(C(C)C)cc(C#N)cc2C(C)C)c(C(=O)OC)c1] |
| 116 | 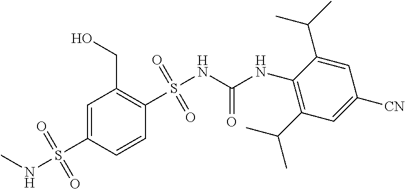 [SMILES: CNS(=O)(=O)c1ccc(S(=O)(=O)NC(=O)Nc2c(C(C)C)cc(C#N)cc2C(C)C)c(CO)c1] |
| 117 | 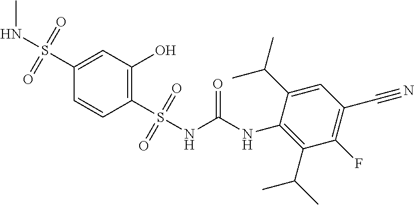 [SMILES: CNS(=O)(=O)c1ccc(S(=O)(=O)NC(=O)Nc2c(C(C)C)cc(C#N)c(F)c2C(C)C)c(O)c1] |
| 118 | 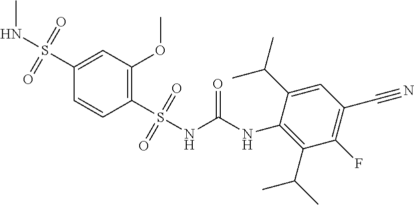 [SMILES: CNS(=O)(=O)c1ccc(S(=O)(=O)NC(=O)Nc2c(C(C)C)cc(C#N)c(F)c2C(C)C)c(OC)c1] |
| 119 | 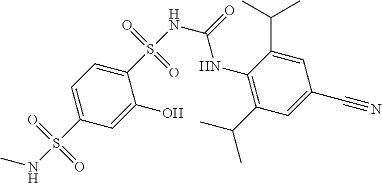 [SMILES: CNS(=O)(=O)c1ccc(S(=O)(=O)NC(=O)Nc2c(C(C)C)cc(C#N)cc2C(C)C)c(O)c1] |
| 120 | 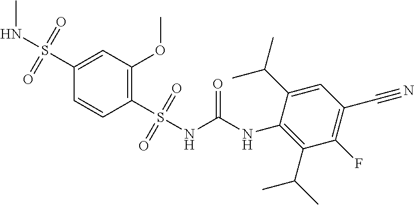 [SMILES: CNS(=O)(=O)c1ccc(S(=O)(=O)NC(=O)Nc2c(C(C)C)cc(C#N)c(F)c2C(C)C)c(OC)c1] |
| 121 | 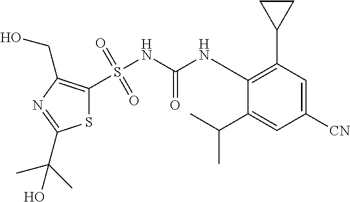 [SMILES: CC(C)c1cc(C#N)cc(C2CC2)c1NC(=O)NS(=O)(=O)c1sc(C(C)(C)O)nc1CO] |
| 122 | 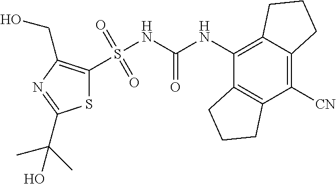 [SMILES: CC(C)(O)c1nc(CO)c(S(=O)(=O)NC(=O)Nc2c3c(c(C#N)c4c2CCC4)CCC3)s1] |
| 123 | 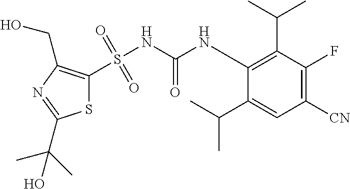 [SMILES: CC(C)c1cc(C#N)c(F)c(C(C)C)c1NC(=O)NS(=O)(=O)c1sc(C(C)(C)O)nc1CO] |
| 124 | 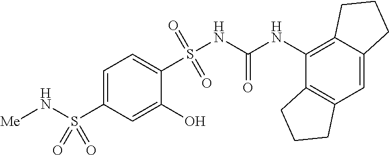 [SMILES: CNS(=O)(=O)c1ccc(S(=O)(=O)NC(=O)Nc2c3c(cc4c2CCC4)CCC3)c(O)c1] |

Compounds having Formula IX, as well as methods of making and using the same, are further described in US Provisional Application entitled “Bis(hydroxalkyl)-S-heteroaryl Sulfonylureas”, filed on even date herewith, which is incorporated herein by reference in its entirety.

In one aspect, an NLRP3 antagonist is a compound of Formula X

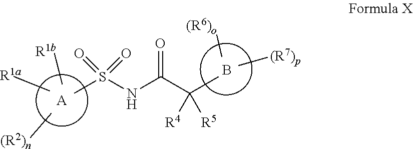

wherein

n=0 or 1;

o=1 or 2;

p=0, 1, 2, or 3;

wherein

A is a 5- to 10-membered heteroaryl or a C₆-C₁₀ aryl;

B is a 5- to 10-membered heteroaryl or a C₆-C₁₀ aryl;

wherein

R^{1a} is —SO₂NR¹¹R¹²;

R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², CN, and —NR¹¹COR¹²;

at least one R⁶ is ortho to the bond connecting the B ring to the CR⁴R⁵ group of Formula X;

R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO (5- to 10-membered heteroaryl), CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), and OCO (3- to 7-membered heterocycloalkyl);

wherein each C₁-C₆ alkyl substituent and each C₁-C₆ alkoxy substituent of the R² C₃-C₇ cycloalkyl or of the R² 3-to 7-membered heterocycloalkyl is further optionally independently substituted with one to three hydroxy, halo, or oxo;

wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3-to 7-membered heterocycloalkyl are optionally substituted with one or more substituents independently selected from halo, C₁-C₆ alkyl, and OC₁-C₆ alkyl;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;

wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

each of R⁴ and R⁵ is independently selected from hydrogen and C₁-C₆ alkyl;

R¹⁰ is C₁-C₆ alkyl;

each of R⁸ and R⁹ at each occurrence is independently selected from hydrogen, C₁-C₆ alkyl, (C═NR¹³)NR¹¹R¹², S(O)₂C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;

R¹³ is C₁-C₆ alkyl, C₆-C₁₀ aryl, or 5- to 10-membered heteroaryl;

each of R¹¹ and R¹² at each occurrence is independently selected from hydrogen and C₁-C₆ alkyl optionally substituted with hydroxy;

with the proviso that the compound of Formula X is not a compound selected from the group consisting of:

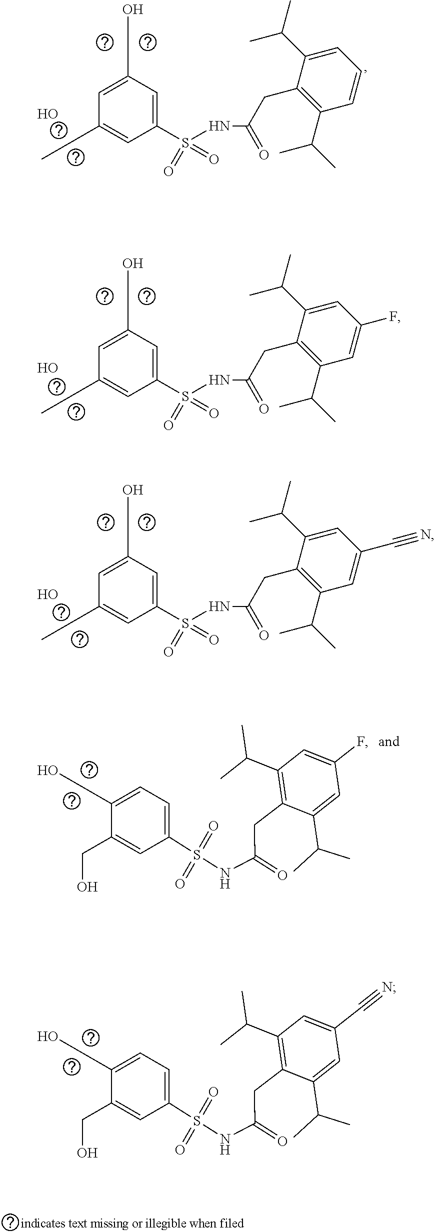

or a pharmaceutically acceptable salt thereof.

In another aspect, an NLRP3 antagonist is a compound of Formula X

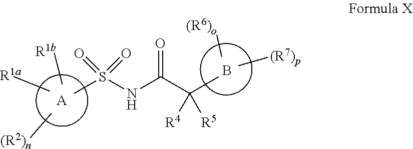

wherein the compound of Formula X is selected from

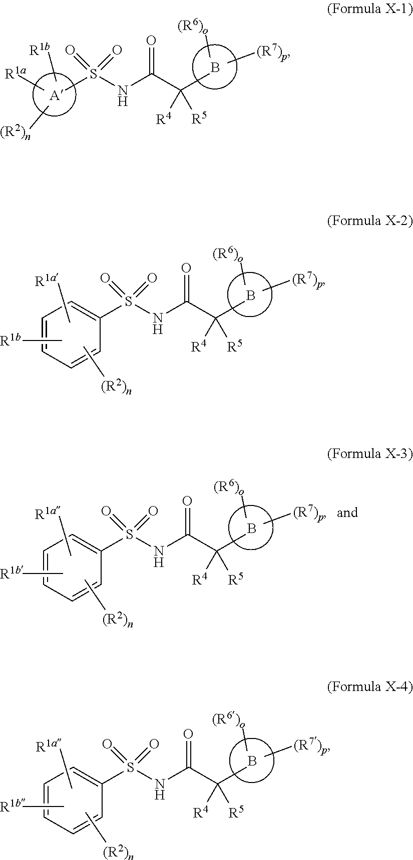

wherein

n=0 or 1;

o=1 or 2;

p=0, 1, 2, or 3;

wherein

A′ is a 5- to 10-membered heteroaryl;

B is a 5- to 10-membered heteroaryl or a C₆-C₁₀ aryl;

wherein

R^{1a} is a C₁-C₆ alkyl, —CR¹¹R¹²NR¹¹R¹² or —SO₂NR¹¹R¹²;
wherein the C₁-C₆ alkyl is substituted with one or more hydroxy or —OSi(R¹³)₃;

R^{1a′} is a C₁-C₆ alkyl, —CR¹¹R¹²NR¹¹R¹² or —SO₂NR¹¹R¹²;
wherein the C₁-C₆ alkyl is substituted with one or more —OSi(R¹³)₃;

R^{1a″} is a C₁-C₆ alkyl;

wherein the C₁-C₆ alkyl is substituted with one or more hydroxy;

R^{1b} is a C₁-C₆ alkyl substituted with one or more hydroxy, —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SO₂R¹³, —NR¹¹CONR¹¹R¹², —CR¹¹R¹²NR¹¹R¹², CN, and —NR¹¹COR¹²;

R^{1b′} is —SO₂NR¹¹R¹², —SO₂R¹³, —CONR¹¹R¹², —OR¹¹, —COR¹³; —CO₂R¹³, —NR¹³CONR¹¹R¹²; —CR¹¹R¹²CN, —NR¹¹SP₂R¹³, —NR¹¹CONR¹¹R¹²—CR¹¹R¹²NR¹¹R¹², —CN, and —NR¹¹COR¹²;

R^{1b″} is a C₁-C₆ alkyl;

wherein the C₁-C₆ alkyl is substituted with one or more hydroxy;

at least one R⁶ is ortho to the bond connecting the B ring to the CR⁴R⁵ group of Formula AA-through Formula AA-1, AA-2, and AA-3;

at least one R^{6′} is ortho to the bond connecting the B ring to the CR⁴R⁵ group of Formula AA-4;

R² is selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl, CO (5- to 10-membered heteroaryl), CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, NHCOOC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), and OCO (3- to 7-membered heterocycloalkyl);

wherein each C₁-C₆ alkyl substituent and each C₁-C₆ alkoxy substituent of the R² C₃-C₇ cycloalkyl or of the R² 3-to 7-membered heterocycloalkyl is further optionally independently substituted with one to three hydroxy, halo, or oxo;

wherein the 3- to 7-membered heterocycloalkyl, C₆-C₁₀ aryl, 5- to 10-membered heteroaryl of the R² C₁-C₆ alkyl, the R² C₁-C₆ haloalkyl, the R² C₃-C₇ cycloalkyl, or the R² 3-to 7-membered heterocycloalkyl are optionally substituted with one or more substituents independently selected from halo, C₁-C₆ alkyl, and OC₁-C₆ alkyl;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, C₆-C₁₀ aryloxy, O(C₃-C₁₀ cycloalkyl), and S(O₂)C₁-C₆ alkyl; and wherein the C₁-C₆ alkyl or C₁-C₆ alkoxy that R⁶ or R⁷ is substituted with is optionally substituted with one or more hydroxyl, halo, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;

wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

R^{6′} and R^{7′} are each independently selected from C₁-C₆ alkyl, C₁-C₆ haloalkyl, C₁-C₆ alkoxy, C₁-C₆ haloalkoxy, Cl, Br, I, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,

wherein R^{6′} and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, C₆-C₁₀ aryloxy, O(C₃-C₁₀ cycloalkyl), and S(O₂)C₁-C₆ alkyl; and wherein the C₁-C₆ alkyl or C₁-C₆ alkoxy that R^{6′} or R⁷ is substituted with is optionally substituted with one or more hydroxyl, halo, C₆-C₁₀ aryl or NR⁸R⁹, or wherein R^{6′} or R⁷ is optionally fused to a five-to-seven-membered carbocyclic ring or heterocyclic ring containing one or two heteroatoms independently selected from oxygen, sulfur and nitrogen;

wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;

or at least one pair of R^{6′} and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

each of R⁴ and R⁵ is independently selected from hydrogen and C₁-C₆ alkyl;

R¹⁰ is C₁-C₆ alkyl;

each of R⁸ and R⁹ at each occurrence is independently selected from hydrogen, C₁-C₆ alkyl, (C═NR¹³)NR¹¹R¹², S(O)₂C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;

R¹³ is C₁-C₆ alkyl, C₆-C₁₀ aryl, or 5- to 10-membered heteroaryl;

each of R¹¹ and R¹² at each occurrence is independently selected from hydrogen and C₁-C₆ alkyl optionally substituted with hydroxy;

or a pharmaceutically acceptable salt thereof.

In some embodiments of the compound of Formula X, the compound of Formula X is

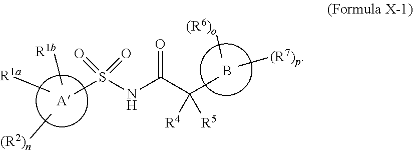

In some embodiments of the compound of Formula X, the compound of Formula X is

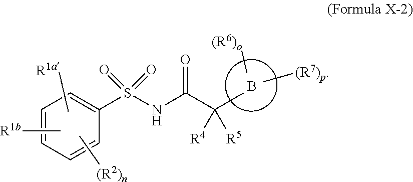

In some embodiments of the compound of Formula X, the compound of Formula X is

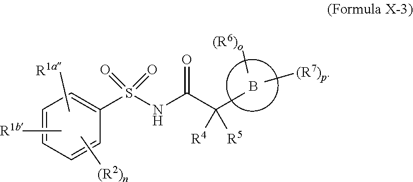

In some embodiments of the compound of Formula X, the compound of Formula AA is

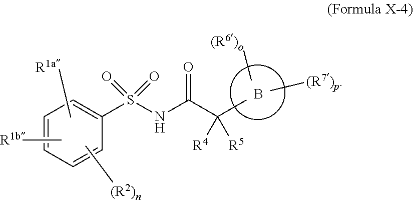

In some embodiments of the compound of Formula X, each of R⁴ and R⁵ is hydrogen.

In some embodiments of the compound of Formula X, one of R⁴ and R⁵ is C₁-C₆ alkyl.

In some embodiments of the compound of Formula X, A is a 5- to 6-membered heteroaryl containing 1 sulfur ring member.

In some embodiments of the compound of Formula X, A is thiazolyl.

In some embodiments of the compound of Formula X, wherein n=0.

In some embodiments of the compound of Formula X, the substituted ring A is

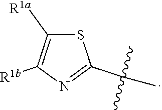

In some embodiments of the compound of Formula X, the substituted ring A is

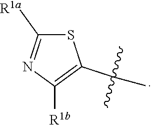

In some embodiments of the compound of Formula X, the substituted ring A is

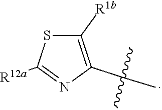

In some embodiments of the compound of Formula X, the substituted ring A is

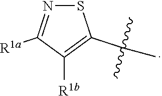

In some embodiments of the compound of Formula X, the substituted ring A is

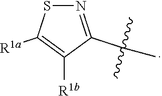

In some embodiments of the compound of Formula X, the substituted ring A is

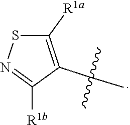

In some embodiments of the compound of Formula X, the substituted ring A is

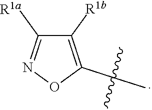

In some embodiments of the compound of Formula X, the substituted ring A is

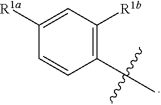

In some embodiments of the compound of Formula X, n=1.

In some embodiments of the compound of Formula X, the substituted ring A is

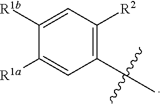

In some embodiments of the compound of Formula X, the substituted ring A is

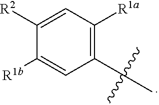

In some embodiments of the compound of Formula X, the substituted ring A is

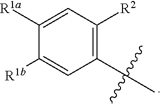

In some embodiments of the compound of Formula X, the substituted ring A is

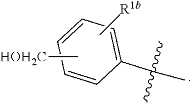

In some embodiments of the compound of Formula X, the substituted ring A is

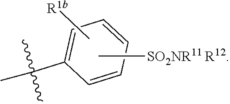

In some embodiments of the compound of Formula X, the substituted ring A is

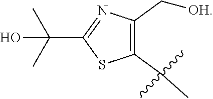 [SMILES: *c1sc(C(C)(C)O)nc1CO]

In some embodiments of the compound of Formula X, the substituted ring A is

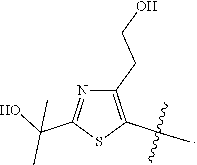 [SMILES: *c1sc(C(C)(C)O)nc1CCO]

In some embodiments of the compound of Formula X, the substituted ring A is

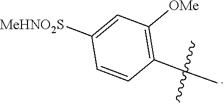

In some embodiments of the compound of Formula X, the substituted ring A is

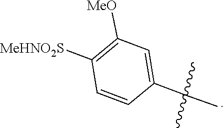

In some embodiments of the compound of Formula X, R^{1a} is C₁-C₆ alkyl substituted with one or more hydroxy.

In some embodiments of the compound of Formula X, R^{1a} is C₁-C₆ alkyl substituted with one or more —OSi(R¹³)₃.

In some embodiments of the compound of Formula X, R^{1a} is —CR¹¹R¹²NR¹¹R¹².

In some embodiments of the compound of Formula X, R^{1a} is —SO₂NR¹¹R¹².

In some embodiments of the compound of Formula X-2, R^{1a′} is C₁-C₆ alkyl substituted with one or more —OSi(R¹³)₃.

In some embodiments of the compound of Formula X-2, R^{1a′} is —CR¹¹R¹²NR¹¹R¹².

In some embodiments of the compound of Formula X-2, R^{1a′} is —SO₂NR¹¹R¹².

In some embodiments of the compound of Formula X, R^{1b} is independently selected from the group consisting of C₁-C₆ alkyl substituted with one or more hydroxy, —SO₂NR¹¹R¹², —SO₂R¹³, —CONR¹¹R¹², —OR¹¹, —COR¹³; —NR¹³CONR¹¹R¹²; —CR¹¹R¹²CN, —NR¹¹SO₂R¹³, —NR¹¹CONR¹¹R¹², and —NR¹¹COR¹²;

In some embodiments of the compound of Formula X, R^{1b} is independently selected from the group consisting of —SO₂NR¹¹R¹², —SO₂R¹³, —CONR¹¹R¹², —COR¹³, —CO₂R¹³, —NR¹³CONR¹¹R¹²; and —CR¹¹R¹²CN.

In some embodiments of the compound of Formula X, R^{1b} is —SO₂NHMe, SO₂NHCH₂CH₂OH, SO₂Me, CONHMe, or OMe.

In some embodiments of the compound of Formula X, R^{1b} is —SO₂NHMe or OMe.

In some embodiments of the compound of Formula X, R² is independently selected from the group consisting of hydroxymethyl, C₂ alkyl substituted with hydroxy, C₃ alkyl substituted with hydroxy, C₄ alkyl substituted with hydroxy, C₅ alkyl substituted with hydroxy, and C₆ alkyl substituted with hydroxy.

In some embodiments of the compound of Formula X, R² is selected from the group consisting of hydroxymethyl, 1-hydroxyethyl, 2-hydroxyethyl, 2-hydroxy-2-propyl, 3-hydroxy-2-propyl, 1-hydroxy-1-propyl, 2-hydroxy-1-propyl, 3-hydroxy-1-propyl, 4-hydroxy-1-butyl, 5-hydroxy-1-pentyl, and 6-hydroxy-1-hexyl.

In some embodiments of the compound of Formula X, R² is selected from the group consisting of hydroxymethyl, 1-hydroxyethyl, 2-hydroxyethyl, 2-hydroxy-2-propyl, 3-hydroxy-2-propyl, 1-hydroxy-1-propyl, 2-hydroxy-1-propyl, 3-hydroxy-1-propyl, 4-hydroxy-1-butyl, and 6-hydroxy-1-hexyl.

In some embodiments of the compound of Formula X, R² is selected from the group consisting of C₁-C₆ alkyl optionally substituted with one or more hydroxy, halo, oxo, or C₁-C₆ alkoxy; C₃-C₇ cycloalkyl optionally substituted with one or more hydroxy, halo, oxo, C₁-C₆ alkoxy, or C₁-C₆ alkyl wherein the C₁-C₆ alkoxy or C₁-C₆ alkyl is further optionally substituted with one to three hydroxy, halo, or oxo; 3- to 7-membered heterocycloalkyl optionally substituted with one or more hydroxy, halo, oxo, or C₁-C₆ alkyl, wherein the C₁-C₆ alkoxy or C₁-C₆ alkyl is further optionally substituted with one to three hydroxy, halo, or oxo; C₁-C₆ haloalkyl; C₁-C₆ alkoxy; C₁-C₆ haloalkoxy; halo; CN; CO—C₁-C₆ alkyl; CO—C₆-C₁₀ aryl; CO (5- to 10-membered heteroaryl); CO₂C₁-C₆ alkyl; CO₂C₃-C₈ cycloalkyl; OCOC₁-C₆ alkyl; OCOC₆-C₁₀ aryl; OCO (5- to 10-membered heteroaryl); OCO (3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; S(O₂)NR¹¹R¹²; S(O)C₁-C₆ alkyl; and S(O₂)C₁-C₆ alkyl.

In some embodiments of the compound of Formula X, R² is selected from the group consisting of fluoro, chloro, cyano, methyl, methoxy, ethoxy, isopropyl, 1-hydroxy-2-methylpropan-2-yl, 2-hydroxy-2-propyl, hydroxymethyl, 1-hydroxyethyl, 2-hydroxyethyl, 1-hydroxy-2-propyl, 1-hydroxy-1-cyclopropyl, COCH₃, COPh, 2-methoxy-2-propyl, phenyl, S(O₂)CH₃, and S(O₂)NR¹¹R¹².

In some embodiments of the compound of Formula X, B is phenyl substituted with 1 or 2 R⁶ and optionally substituted with 1, 2, or 3 R⁷.

In certain embodiments of Formula X (when B is phenyl substituted with 1 or 2 R⁶ and optionally substituted with 1, 2, or 3 R⁷), o=2 and p=0.

In certain embodiments of Formula X (when B is phenyl substituted with 1 or 2 R⁶ and optionally substituted with 1, 2, or 3 R⁷), the optionally substituted ring B is

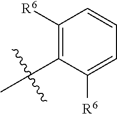

In certain of the foregoing embodiments of Formula X, each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl.

As non-limiting examples of the foregoing, each R⁶ can be independently selected from the group consisting of: C₁-C₆ alkyl, C₃-C₇ cycloalkyl, C₁-C₆ haloalkyl, C₁-C₆ alkoxy, and C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.

In certain embodiments of Formula X (when B is phenyl substituted with 1 or 2 R⁶ and optionally substituted with 1, 2, or 3 R⁷), o=1 and p=1.

In certain embodiments of Formula X (when B is phenyl substituted with 1 or 2 R⁶ and optionally substituted with 1, 2, or 3 R⁷), o=2 and p=1.

In certain of the foregoing embodiments of Formula X (when o=2 and p=1), the optionally substituted ring B is

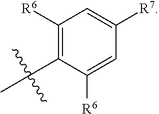

In certain of the foregoing embodiments of Formula X (when the optionally substituted ring B is

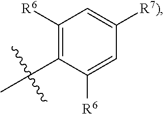

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;

wherein IC is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

In certain embodiments of Formula X (when B is phenyl substituted with 1 or 2 R⁶ and optionally substituted with 1, 2, or 3 R⁷), o=2 and p=2.

In some embodiments of Formula X, the optionally substituted ring B is

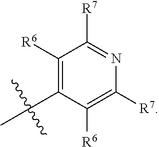

In certain embodiments of the foregoing (when o=2 and p=2 and/or when the optionally substituted ring B

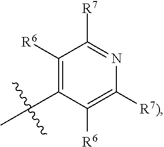

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;

wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain embodiments of Formula X (when o=2 and p=2), the optionally substituted ring B is

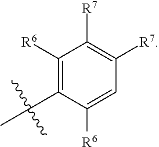

In certain of the foregoing embodiments (when the optionally substituted ring B is

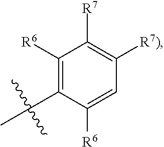

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;

wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, NR¹⁰ COOC₁-C₆ alkyl, C₆-C₁₀ aryl, and CONR⁸R⁹.

In certain embodiments of Formula X (when B is phenyl substituted with 1 or 2 R⁶ and optionally substituted with 1, 2, or 3 R⁷), o=2 and p=3

In certain of the foregoing embodiments (when o=2 and p=3), the optionally substituted ring B is

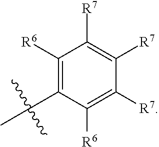

In certain of the foregoing embodiments (when the optionally substituted ring B is

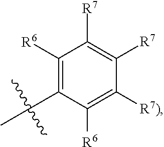

the optionally substituted ring B is

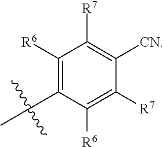

In some embodiments of Formula X, the optionally substituted ring B is

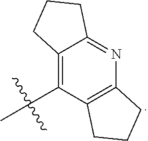 [SMILES: *c1c2c(nc3c1CCC3)CCC2]

In certain embodiments of the foregoing (when the optionally substituted ring B is

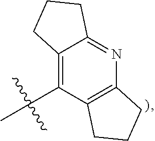 [SMILES: *c1c2c(nc3c1CCC3)CCC2]

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;

wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments of the compound of Formula X, two pairs of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments of the compound of Formula X, each R⁶ is independently selected from CN, C₁-C₆ alkyl, 5- to 10-membered heteroaryl, and 3- to 7-membered heterocycloalkyl;

wherein the C₁-C₆ alkyl is optionally substituted with one or more substituents each independently selected from hydroxyl or C₁-C₆ alkoxy.

In some embodiments of the compound of Formula X, each R⁷ is independently selected from CN, C₁-C₆ alkyl, 5- to 10-membered heteroaryl, and 3- to 7-membered heterocycloalkyl;

wherein the C₁-C₆ alkyl is optionally substituted with one or more substituents each independently selected from hydroxyl or C₁-C₆ alkoxy.

In some embodiments of the compound of Formula X, R³ is hydrogen.

In another aspect, an NLRP3 antagonist is a compound selected from the group consisting of the compounds in Table 10 below and pharmaceutically acceptable salts thereof.

TABLE 10

| Compound | Structure |
|---|---|
| 101 | 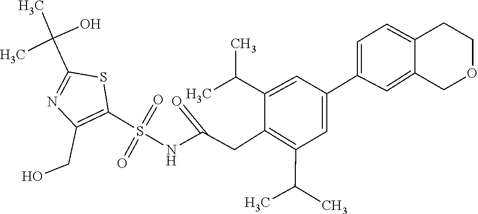 [SMILES: CC(C)c1cc(-c2ccc3c(c2)COCC3)cc(C(C)C)c1CC(=O)NS(=O)(=O)c1sc(C(C)(C)O)nc1CO] |
| 102 | 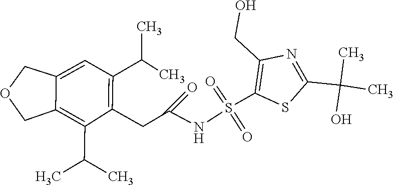 [SMILES: CC(C)c1cc2c(c(C(C)C)c1CC(=O)NS(=O)(=O)c1sc(C(C)(C)O)nc1CO)COC2] |
| 103 | 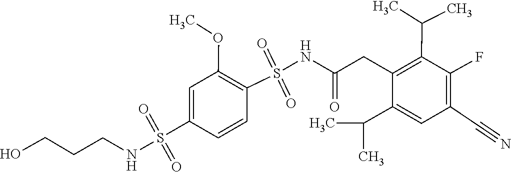 [SMILES: COc1cc(S(=O)(=O)NCCCO)ccc1S(=O)(=O)NC(=O)Cc1c(C(C)C)cc(C#N)c(F)c1C(C)C] |
| 104 | 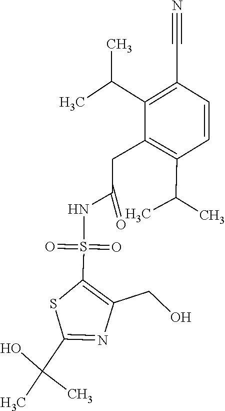 [SMILES: CC(C)c1ccc(C#N)c(C(C)C)c1CC(=O)NS(=O)(=O)c1sc(C(C)(C)O)nc1CO] |
| 105 | 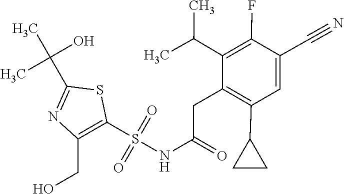 [SMILES: CC(C)c1c(F)c(C#N)cc(C2CC2)c1CC(=O)NS(=O)(=O)c1sc(C(C)(C)O)nc1CO] |
| 106 | 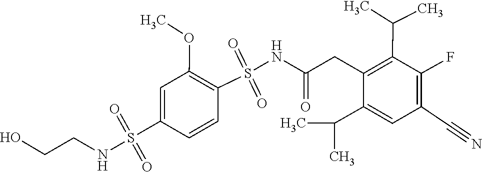 [SMILES: COc1cc(S(=O)(=O)NCCO)ccc1S(=O)(=O)NC(=O)Cc1c(C(C)C)cc(C#N)c(F)c1C(C)C] |
| 107 | 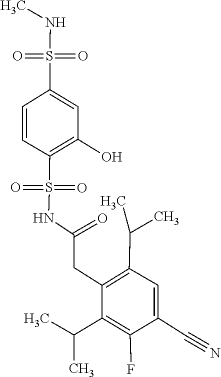 [SMILES: CNS(=O)(=O)c1ccc(S(=O)(=O)NC(=O)Cc2c(C(C)C)cc(C#N)c(F)c2C(C)C)c(O)c1] |
| 108 | 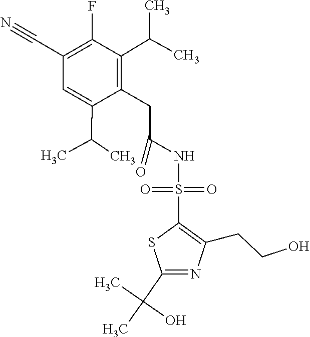 [SMILES: CC(C)c1cc(C#N)c(F)c(C(C)C)c1CC(=O)NS(=O)(=O)c1sc(C(C)(C)O)nc1CCO] |
| 109 | 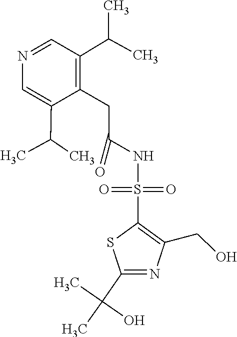 [SMILES: CC(C)c1cncc(C(C)C)c1CC(=O)NS(=O)(=O)c1sc(C(C)(C)O)nc1CO] |
| 110 | 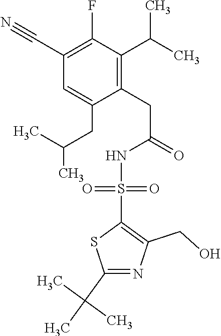 [SMILES: CC(C)Cc1cc(C#N)c(F)c(C(C)C)c1CC(=O)NS(=O)(=O)c1sc(C(C)(C)C)nc1CO] |
| 111 | 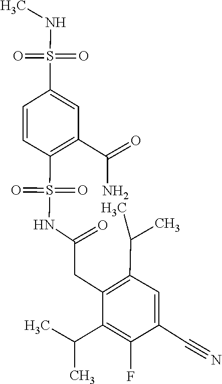 [SMILES: CNS(=O)(=O)c1ccc(S(=O)(=O)NC(=O)Cc2c(C(C)C)cc(C#N)c(F)c2C(C)C)c(C(N)=O)c1] |
| 112 | 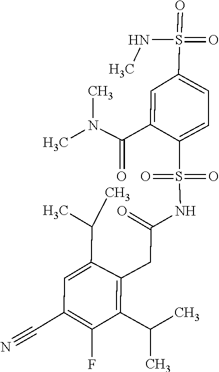 [SMILES: CNS(=O)(=O)c1ccc(S(=O)(=O)NC(=O)Cc2c(C(C)C)cc(C#N)c(F)c2C(C)C)c(C(=O)N(C)C)c1] |
| 114 | 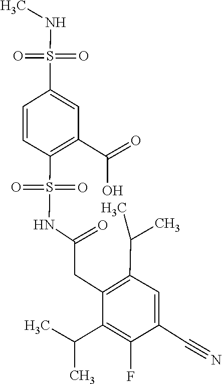 [SMILES: CNS(=O)(=O)c1ccc(S(=O)(=O)NC(=O)Cc2c(C(C)C)cc(C#N)c(F)c2C(C)C)c(C(=O)O)c1] |
| 115 | 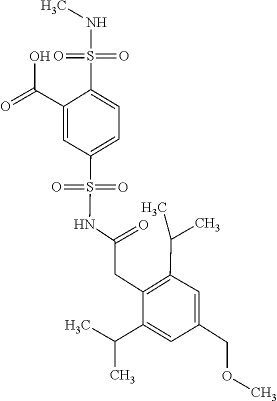 [SMILES: CNS(=O)(=O)c1ccc(S(=O)(=O)NC(=O)Cc2c(C(C)C)cc(COC)cc2C(C)C)cc1C(=O)O] |
| 116 | 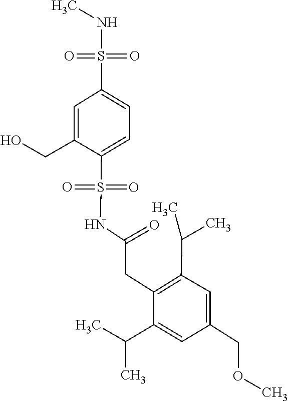 [SMILES: CNS(=O)(=O)c1ccc(S(=O)(=O)NC(=O)Cc2c(C(C)C)cc(COC)cc2C(C)C)c(CO)c1] |
| 117 | 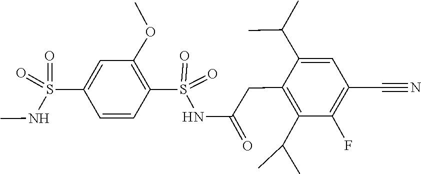 |
| 118 | 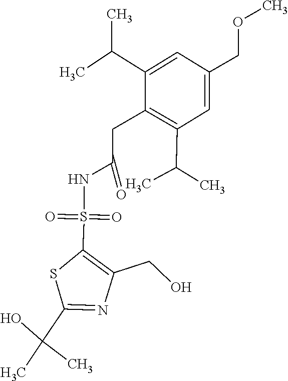 [SMILES: COCc1cc(C(C)C)c(CC(=O)NS(=O)(=O)c2sc(C(C)(C)O)nc2CO)c(C(C)C)c1] |
| 119 | 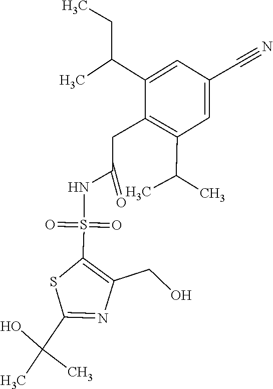 [SMILES: CCC(C)c1cc(C#N)cc(C(C)C)c1CC(=O)NS(=O)(=O)c1sc(C(C)(C)O)nc1CO] |
| 120 | 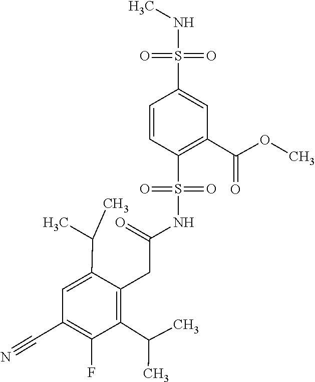 [SMILES: CNS(=O)(=O)c1ccc(S(=O)(=O)NC(=O)Cc2c(C(C)C)cc(C#N)c(F)c2C(C)C)c(C(=O)OC)c1] |
| 121 | 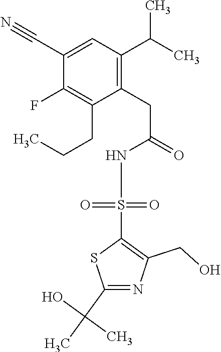 [SMILES: CCCc1c(F)c(C#N)cc(C(C)C)c1CC(=O)NS(=O)(=O)c1sc(C(C)(C)O)nc1CO] |
| 122 | 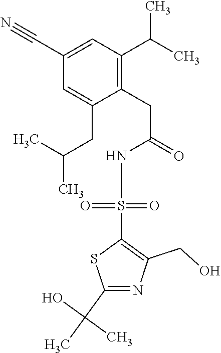 [SMILES: CC(C)Cc1cc(C#N)cc(C(C)C)c1CC(=O)NS(=O)(=O)c1sc(C(C)(C)O)nc1CO] |
| 123 | 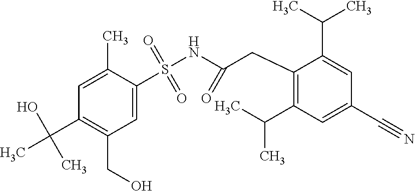 [SMILES: Cc1cc(C(C)(C)O)c(CO)cc1S(=O)(=O)NC(=O)Cc1c(C(C)C)cc(C#N)cc1C(C)C] |
| 124 | 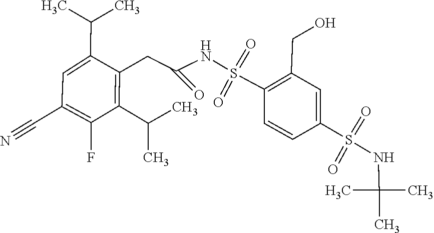 [SMILES: CC(C)c1cc(C#N)c(F)c(C(C)C)c1CC(=O)NS(=O)(=O)c1ccc(S(=O)(=O)NC(C)(C)C)cc1CO] |
| 125 | 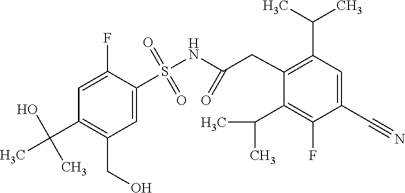 [SMILES: CC(C)c1cc(C#N)c(F)c(C(C)C)c1CC(=O)NS(=O)(=O)c1cc(CO)c(C(C)(C)O)cc1F] |
| 126 | 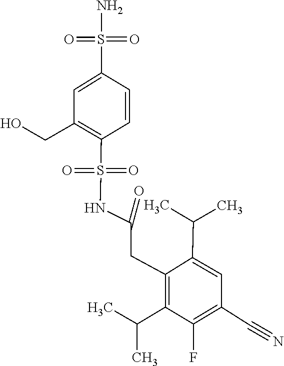 [SMILES: CC(C)c1cc(C#N)c(F)c(C(C)C)c1CC(=O)NS(=O)(=O)c1ccc(S(N)(=O)=O)cc1CO] |
| 127 | 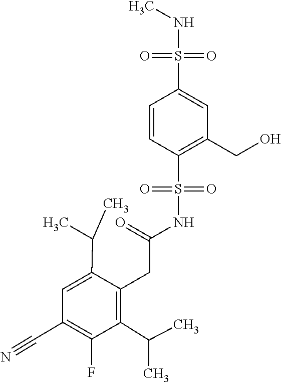 [SMILES: CNS(=O)(=O)c1ccc(S(=O)(=O)NC(=O)Cc2c(C(C)C)cc(C#N)c(F)c2C(C)C)c(CO)c1] |
| 128 | 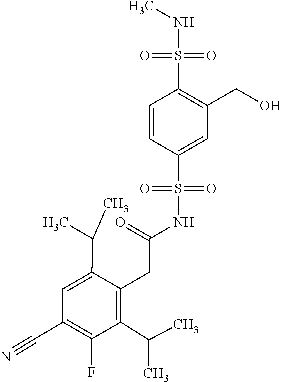 [SMILES: CNS(=O)(=O)c1ccc(S(=O)(=O)NC(=O)Cc2c(C(C)C)cc(C#N)c(F)c2C(C)C)cc1CO] |
| 129 | 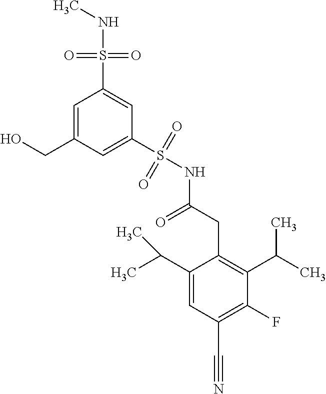 [SMILES: CNS(=O)(=O)c1cc(CO)cc(S(=O)(=O)NC(=O)Cc2c(C(C)C)cc(C#N)c(F)c2C(C)C)c1] |
| 130 | 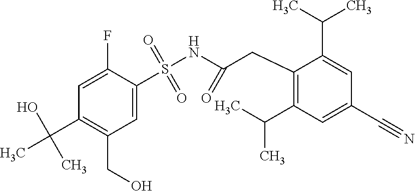 [SMILES: CC(C)c1cc(C#N)cc(C(C)C)c1CC(=O)NS(=O)(=O)c1cc(CO)c(C(C)(C)O)cc1F] |
| 131 | 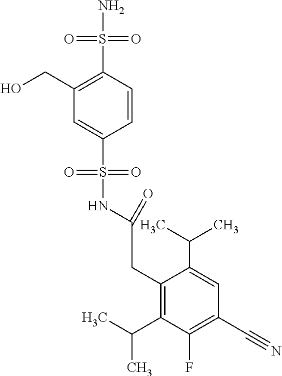 [SMILES: CC(C)c1cc(C#N)c(F)c(C(C)C)c1CC(=O)NS(=O)(=O)c1ccc(S(N)(=O)=O)c(CO)c1] |
| 132 | 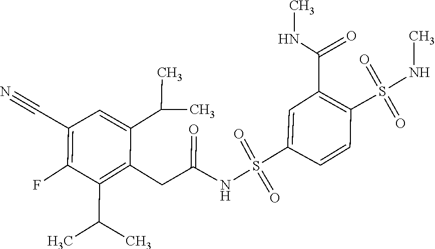 [SMILES: CNC(=O)c1cc(S(=O)(=O)NC(=O)Cc2c(C(C)C)cc(C#N)c(F)c2C(C)C)ccc1S(=O)(=O)NC] |
| 133 | 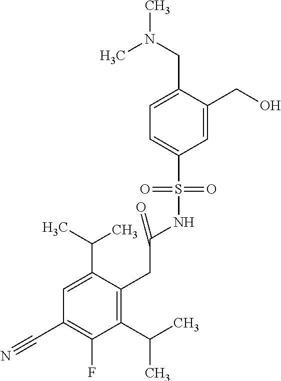 [SMILES: CC(C)c1cc(C#N)c(F)c(C(C)C)c1CC(=O)NS(=O)(=O)c1ccc(CN(C)C)c(CO)c1] |
| 134 | 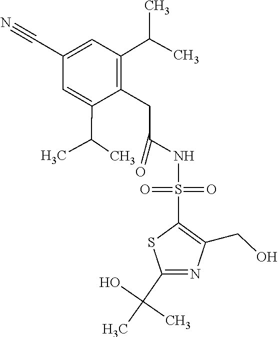 [SMILES: CC(C)c1cc(C#N)cc(C(C)C)c1CC(=O)NS(=O)(=O)c1sc(C(C)(C)O)nc1CO] |
| 135 | 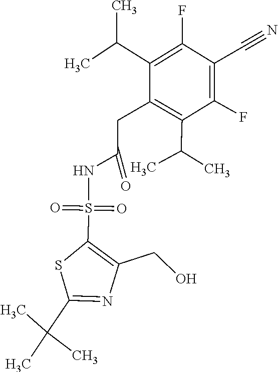 [SMILES: CC(C)c1c(F)c(C#N)c(F)c(C(C)C)c1CC(=O)NS(=O)(=O)c1sc(C(C)(C)C)nc1CO] |
| 136 | 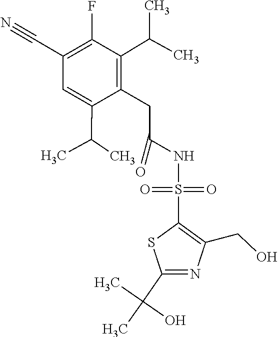 [SMILES: CC(C)c1cc(C#N)c(F)c(C(C)C)c1CC(=O)NS(=O)(=O)c1sc(C(C)(C)O)nc1CO] |
| 137 | 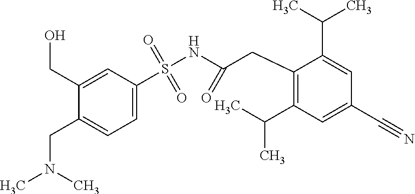 [SMILES: CC(C)c1cc(C#N)cc(C(C)C)c1CC(=O)NS(=O)(=O)c1ccc(CN(C)C)c(CO)c1] |
| 138 | 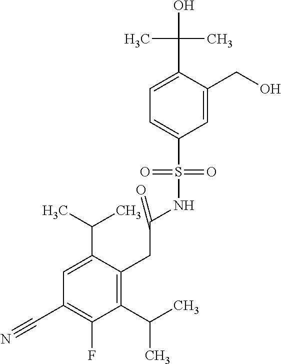 [SMILES: CC(C)c1cc(C#N)c(F)c(C(C)C)c1CC(=O)NS(=O)(=O)c1ccc(C(C)(C)O)c(CO)c1] |
| 139 | 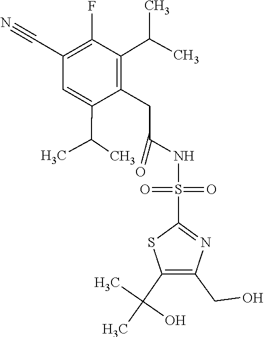 [SMILES: CC(C)c1cc(C#N)c(F)c(C(C)C)c1CC(=O)NS(=O)(=O)c1nc(CO)c(C(C)(C)O)s1] |
| 140 | 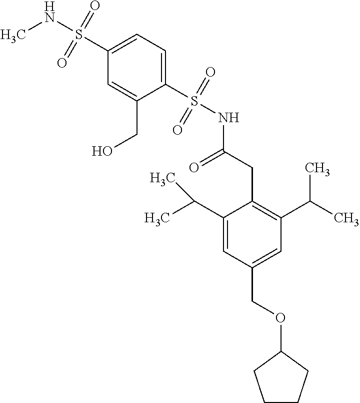 [SMILES: CNS(=O)(=O)c1ccc(S(=O)(=O)NC(=O)Cc2c(C(C)C)cc(COC3CCCC3)cc2C(C)C)c(CO)c1] |
| 141 | 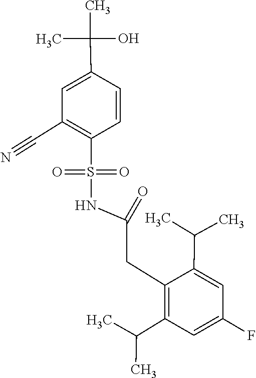 [SMILES: CC(C)c1cc(F)cc(C(C)C)c1CC(=O)NS(=O)(=O)c1ccc(C(C)(C)O)cc1C#N] |
| 142 | 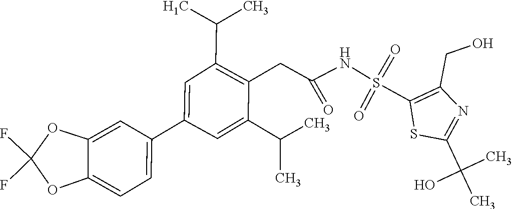 |

Compounds having Formula X, as well as methods of making and using the same, are further described in US Provisional Application entitled “Sulfonamido-substituted N-acyl Sulfonamides”, filed on even date herewith, which is incorporated herein by reference in its entirety.

In one aspect, an NLRP3 antagonist is a compound of Formula XI

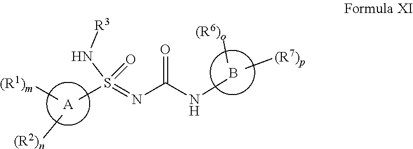

wherein

m=0, 1, or 2;

n=0, 1, or 2;

=1 or 2;

p=0, 1, 2, or 3,

wherein

A is a 5- to 10-membered monocyclic or bicyclic heteroaryl or a C₆-C₁₀ monocyclic or bicyclic aryl;

B is a 5-membered heteroaryl, a 7-10 membered monocyclic or bicyclic heteroaryl, or a C₆-C₁₀ monocyclic or bicyclic aryl;

wherein

at least one R⁶ is ortho to the bond connecting the B ring to the NH(CO) group of Formula AA;

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl; CO (5- to 10-membered heteroaryl); CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3-to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;

or at least one pair of R¹ and R² on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

each of R⁴ and R⁵ is independently selected from hydrogen and C₁-C₆ alkyl;

R¹⁰ is C₁-C₆ alkyl;

each of R⁸ and R⁹ at each occurrence is independently selected from hydrogen, C₁-C₆ alkyl, (C═NR¹³)NR¹¹R¹². S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;

R¹³ is C₁-C₆ alkyl, C₆-C₁₀ aryl, or 5- to 10-membered heteroaryl;

each of R¹¹ and R¹² at each occurrence is independently selected from hydrogen and C₁-C₆ alkyl;

R³ is selected from hydrogen, cyano, hydroxy, C₁-C₆ alkoxy, C₁-C₆ alkyl, and

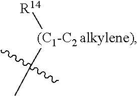

wherein the C₁-C₂ alkylene group is optionally substituted by oxo; and

R¹⁴ is hydrogen, C₁-C₆ alkyl, 5- to 10-membered monocyclic or bicyclic heteroaryl or C₆-C₁₀ monocyclic or bicyclic aryl, wherein each C₁-C₆ alkyl, aryl or heteroaryl is optionally independently substituted with 1 or 2 R⁶,

or a pharmaceutically acceptable salt thereof.

In some embodiments of the compound of Formula XI, A is a 5- to 6-membered monocyclic heteroaryl optionally substituted with 1 or 2 R¹ and optionally substituted with 1 or 2 R².

In some embodiments of the compound of Formula XI, A is furanyl optionally substituted with 1 or 2 R¹ and optionally substituted with 1 or 2 R².

In some embodiments of the compound of Formula XI, A is thiophenyl optionally substituted with 1 or 2 R¹ and optionally substituted with 1 or 2 R².

In some embodiments of the compound of Formula XI, A is oxazolyl optionally substituted with 1 or 2 R¹ and optionally substituted with 1 or 2 R².

In some embodiments of the compound of Formula XI, A is thiazolyl optionally substituted with 1 or 2 R¹ and optionally substituted with 1 or 2 R².

In some embodiments of the compound of Formula XI, A is phenyl optionally substituted with 1 or 2 R¹ and optionally substituted with 1 or 2 R².

In some embodiments of the compound of Formula XI, m=1 and n=0.

In some embodiments of the compound of Formula XI, A is

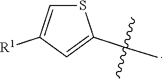

In some embodiments of the compound of Formula XI, A is

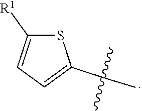

In some embodiments of the compound of Formula XI, A is

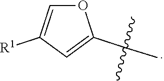

In some embodiments of the compound of Formula XI, A is

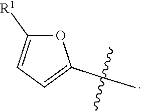

In some embodiments of the compound of Formula XI, A is

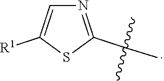

In some embodiments of the compound of Formula XI, A is

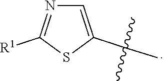

In some embodiments of the compound of Formula XI, A is

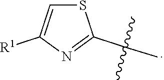

In some embodiments of the compound of Formula XI, A is

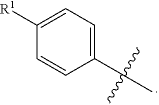

In some embodiments of the compound of Formula XI, A is

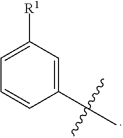

In some embodiments of the compound of Formula XI, A is

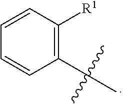

In some embodiments of the compound of Formula XI, A is

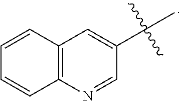 [SMILES: *c1cnc2ccccc2c1]

In some embodiments of the compound of Formula XI, A is

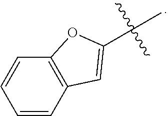 [SMILES: *c1cc2ccccc2o1]

In some embodiments of the compound of Formula XI, A is

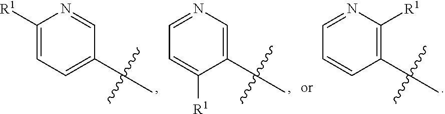

In some embodiments of the compound of Formula XI, A is

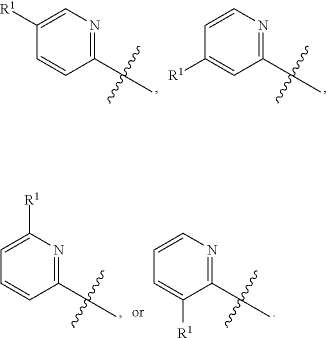

In some embodiments of the compound of Formula XI, A is

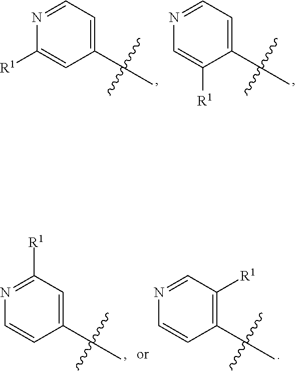

In some embodiments of the compound of Formula XI, m=1 and n=1.

In some embodiments of the compound of Formula XI, A is

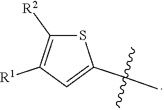

In some embodiments of the compound of Formula XI, A is

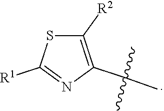

In some embodiments of the compound of Formula XI, A is

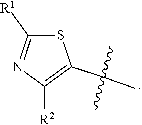

In some embodiments of the compound of Formula XI, A is

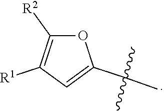

In some embodiments of the compound of Formula XI, A is

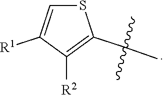

In some embodiments of the compound of Formula XI, A is

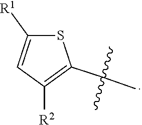

In some embodiments of the compound of Formula XI, A is

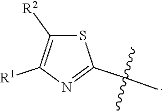

In some embodiments of the compound of Formula XI, A is

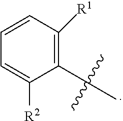

In some embodiments of the compound of Formula XI, A is

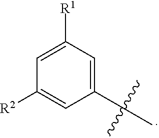

In some embodiments of the compound of Formula XI, A is

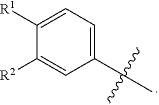

In some embodiments of the compound of Formula XI, A is

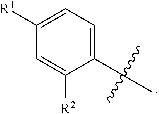

In some embodiments of the compound of Formula XI, A is

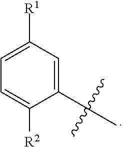

In some embodiments of the compound of Formula XI, m=2 and n=1.

In some embodiments of the compound of Formula XI, A

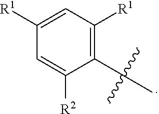

In some embodiments of the compound of Formula XI, A is

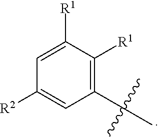

In some embodiments of the compound of Formula XI, A is

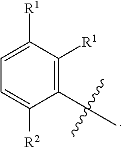

In some embodiments of the compound of Formula XI, A is

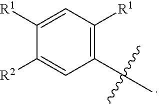

In some embodiments of the compound of Formula XI, each of R¹ and R², when present, is independently selected from the group consisting of C₁-C₆ alkyl optionally substituted with one or more hydroxy, halo, oxo, C₁-C₆ alkoxy, or NR⁸R⁹; C₃-C₇ cycloalkyl optionally substituted with one or more hydroxy, halo, oxo, C₁-C₆ alkoxy, C₁-C₆ alkyl, or NR⁸R⁹ wherein the C₁-C₆ salkoxy or C₁-C₆ alkyl is further optionally substituted with one to three hydroxy, halo, NR⁸R⁹, or oxo; 3- to 7-membered heterocycloalkyl optionally substituted with one or more hydroxy, halo, oxo, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; C₁-C₆ haloalkyl; C₁-C₆ alkoxy; C₁-C₆ haloalkoxy; halo; CN; CO—C₁-C₆ alkyl; CO—C₆-C₁₀ aryl; CO (5- to 10-membered heteroaryl); CO₂C₁-C₆ alkyl; CO₂C₃-C₈ cycloalkyl; OCOC₁-C₆ alkyl; OCOC₆-C₁₀ aryl; OCO (5- to 10-membered heteroaryl); OCO (3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; S(O₂)NR¹¹R¹²; S(O)C₁-C₆ alkyl; and S(O₂)C₁-C₆ alkyl.

In some embodiments of the compound of Formula XI, R¹ is selected from the group consisting of 1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; S(O₂)CH₃, and S(O₂)NR¹¹R¹².

In certain embodiments of the foregoing (when each of R¹ and R², when present, is independently selected from the group consisting of C₁-C₆ alkyl optionally substituted with one or more hydroxy, halo, oxo, C₁-C₆ alkoxy, or NR⁸R⁹; C₃-C₇ cycloalkyl optionally substituted with one or more hydroxy, halo, oxo, C₁-C₆ alkoxy, C₁-C₆ alkyl, or NR⁸R⁹ wherein the C₁-C₆ salkoxy or C₁-C₆ alkyl is further optionally substituted with one to three hydroxy, halo, NR⁸R⁹, or oxo; 3- to 7-membered heterocycloalkyl optionally substituted with one or more hydroxy, halo, oxo, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; C₁-C₆ haloalkyl; C₁-C₆ alkoxy; C₁-C₆ haloalkoxy; halo; CN; CO—C₁-C₆ alkyl; CO—C₆-C₁₀ aryl; CO (5- to 10-membered heteroaryl); CO₂C₁-C₆ alkyl; CO₂C₃-C₈ cycloalkyl; OCOC₁-C₆ alkyl; OCOC₆-C₁₀ aryl; OCO (5- to 10-membered heteroaryl); OCO (3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; S(O₂)NR¹¹R¹²; S(O)C₁-C₆ alkyl; and S(O₂)C₁-C₆ alkyl; or when R¹ is selected from the group consisting of 1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl), R² is selected from the group consisting of fluoro, chloro, cyano, methyl; methoxy; ethoxy; isopropyl; 1-hydroxy-2-methylpropan-2-yl; 2-hydroxy-2-propyl; hydroxymethyl; 1-hydroxyethyl; 2-hydroxyethyl; 1-hydroxy-2-propyl; 1-hydroxy-1-cyclopropyl; COCH₃; COPh; 2-methoxy-2-propyl; (dimethylamino)methyl; S(O₂)CH₃, and S(O₂)NR¹¹R¹².

In some embodiments of the compound of Formula XI, B is phenyl substituted with 1 or 2 R⁶ and optionally substituted with 1, 2, or 3 R⁷.

In certain embodiments (when B is phenyl substituted with 1 or 2 R⁶ and optionally substituted with 1, 2, or 3 R⁷), o=2 and p=0.

In certain embodiments (when B is phenyl substituted with 1 or 2 R⁶ and optionally substituted with 1, 2, or 3 R⁷ (e.g., when B is phenyl; and o=2 and p=o)), B is

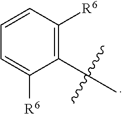

In certain embodiments (when B is

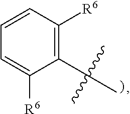

each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl.

In certain embodiments (when B is

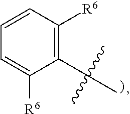

each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.

In certain embodiments (when B is phenyl substituted with 1 or 2 R⁶ and optionally substituted with 1, 2, or 3 R⁷), o=1 and p=1.

In certain embodiments (when B is phenyl substituted with 1 or 2 R⁶ and optionally substituted with 1, 2, or 3 R⁷), o=2 and p=1.

In certain embodiments (when B is phenyl substituted with 1 or 2 R⁶ and optionally substituted with 1, 2, or 3 R⁷; and o=2 and p=1), B is

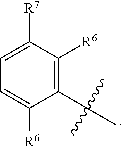

In certain embodiments (when B is

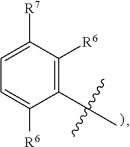

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain embodiments (when B is phenyl substituted with 1 or 2 R⁶ and optionally substituted with 1, 2, or 3 R⁷; and o=2 and p=1), B is

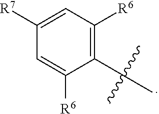

In certain embodiments (when B is

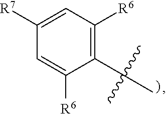

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

In certain embodiments (when B is phenyl substituted with 1 or 2 R⁶ and optionally substituted with 1, 2, or 3 R⁷), o=2 and p=2.

In certain embodiments (when B is phenyl substituted with 1 or 2 R⁶ and optionally substituted with 1, 2, or 3 R⁷; and o=2 and p=2), B is

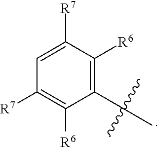

In certain embodiments (when B is

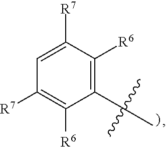

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, NR¹⁰ COOC₁-C₆ alkyl, C₆-C₁₀ aryl, and CONR⁸R⁹.

In some embodiments (when B is phenyl substituted with 1 or 2 R⁶ and optionally substituted with 1, 2, or 3 R⁷; and o=2 and p=2), B is

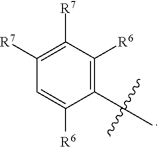

In some embodiments (when B is

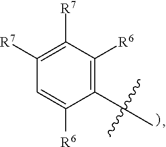

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, NR¹⁰ COOC₁-C₆ alkyl, C₆-C₁₀ aryl, and CONR⁸R⁹.

In certain embodiments (when B is phenyl substituted with 1 or 2 R⁶ and optionally substituted with 1, 2, or 3 R⁷), o=2 and p=3

In certain embodiments (when B is phenyl substituted with 1 or 2 R⁶ and optionally substituted with 1, 2, or 3 R⁷; and o=2 and p=3), B is

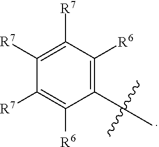

In certain embodiments (when B is

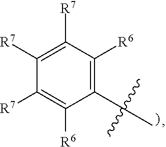

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments of the compound of Formula XI, R³ is hydrogen.

In another aspect, an NLRP3 antagonist is a compound selected from the group consisting of the compounds in Table 11 below:

TABLE 11

| Compound | Structure |
|---|---|
| 101' | 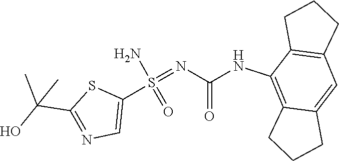 [SMILES: CC(C)(O)c1ncc(S(N)(=O)=NC(=O)Nc2c3c(cc4c2CCC4)CCC3)s1] |
| 101 | 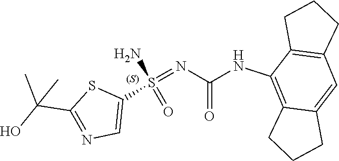 |
| 102 | 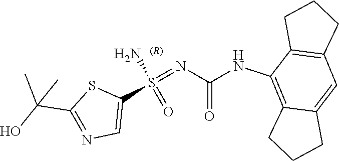 |
| 103' | 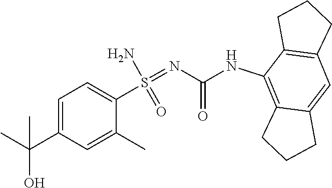 [SMILES: Cc1cc(C(C)(C)O)ccc1S(N)(=O)=NC(=O)Nc1c2c(cc3c1CCC3)CCC2] |
| 103 | 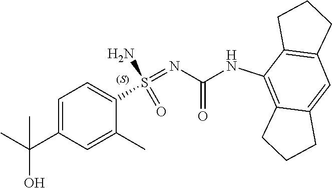 |
| 104 | 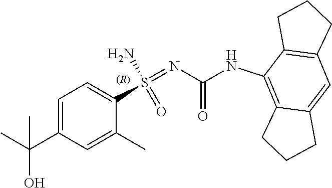 |
| 105 | 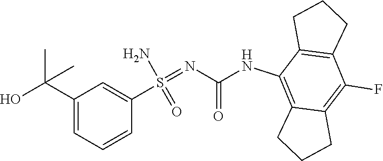 [SMILES: CC(C)(O)c1cccc(S(N)(=O)=NC(=O)Nc2c3c(c(F)c4c2CCC4)CCC3)c1] |
| 105a | 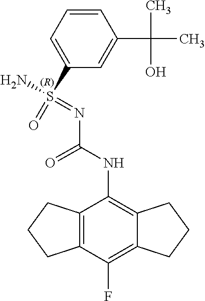 |
| 105b | 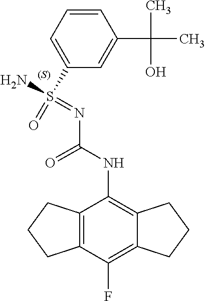 |
| 106 | 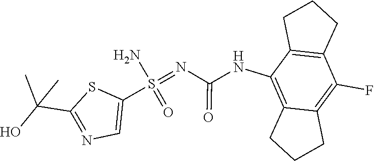 [SMILES: CC(C)(O)c1ncc(S(N)(=O)=NC(=O)Nc2c3c(c(F)c4c2CCC4)CCC3)s1] |
| 106a | 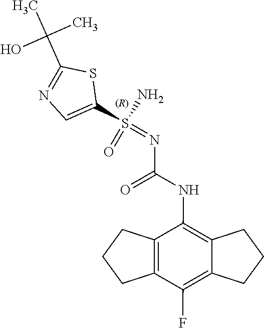 |
| 106b | 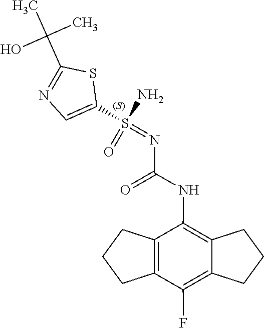 |
| 107 | 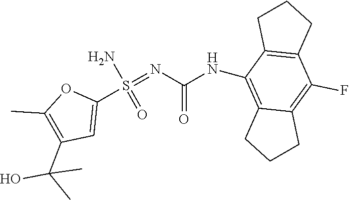 [SMILES: Cc1oc(S(N)(=O)=NC(=O)Nc2c3c(c(F)c4c2CCC4)CCC3)cc1C(C)(C)O] |
| 107a | 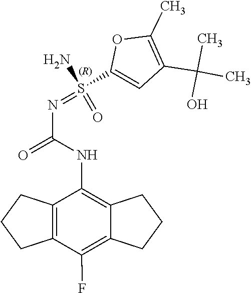 |
| 107b | 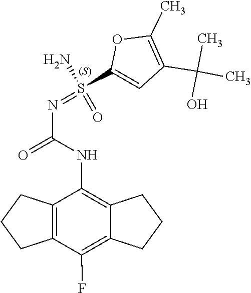 |
| 108 | 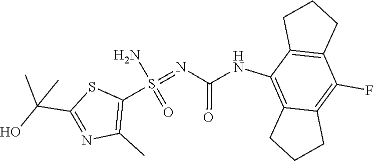 [SMILES: Cc1nc(C(C)(C)O)sc1S(N)(=O)=NC(=O)Nc1c2c(c(F)c3c1CCC3)CCC2] |
| 108a | 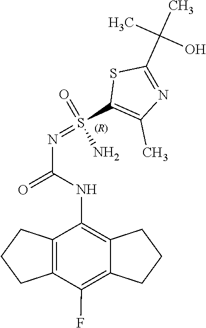 |
| 108b | 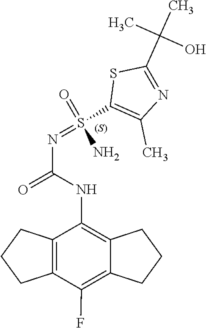 |
| 109 | 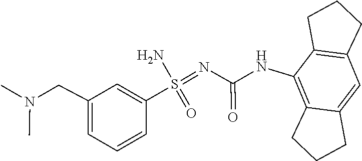 [SMILES: CN(C)Cc1cccc(S(N)(=O)=NC(=O)Nc2c3c(cc4c2CCC4)CCC3)c1] |
| 109a | 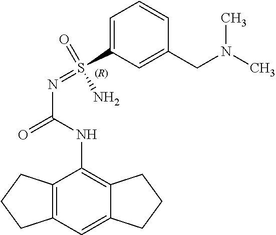 |
| 109b | 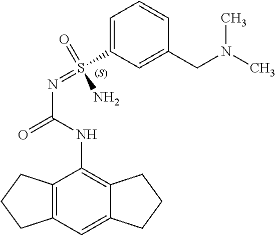 |
| 110 | 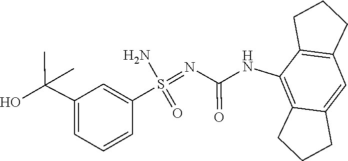 [SMILES: CC(C)(O)c1cccc(S(N)(=O)=NC(=O)Nc2c3c(cc4c2CCC4)CCC3)c1] |
| 110a | 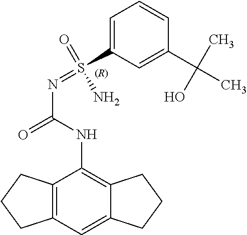 |
| 110b | 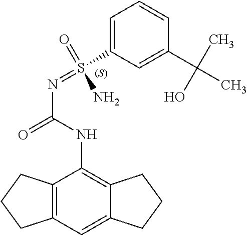 |
| 111 | 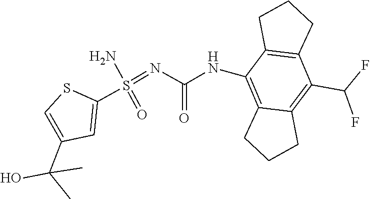 [SMILES: CC(C)(O)c1csc(S(N)(=O)=NC(=O)Nc2c3c(c(C(F)F)c4c2CCC4)CCC3)c1] |
| 112 | 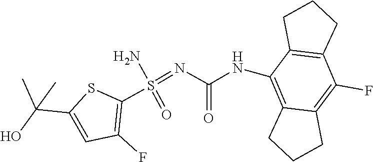 [SMILES: CC(C)(O)c1cc(F)c(S(N)(=O)=NC(=O)Nc2c3c(c(F)c4c2CCC4)CCC3)s1] |
| 112a | 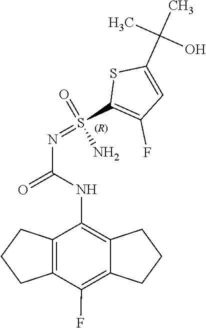 |
| 112b | 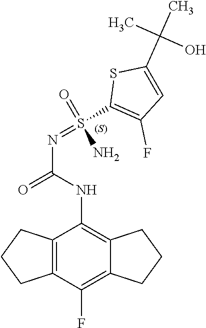 |
| 113 | 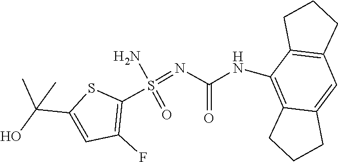 [SMILES: CC(C)(O)c1cc(F)c(S(N)(=O)=NC(=O)Nc2c3c(cc4c2CCC4)CCC3)s1] |
| 113a | 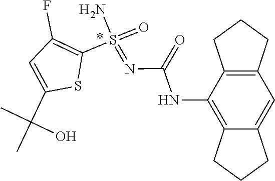 |
| 113b | 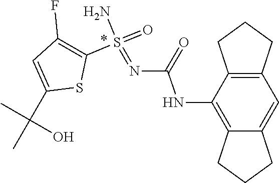 |
| 114 | 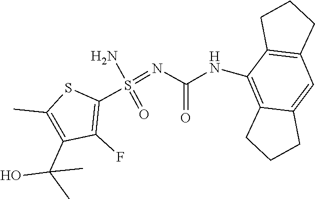 [SMILES: Cc1sc(S(N)(=O)=NC(=O)Nc2c3c(cc4c2CCC4)CCC3)c(F)c1C(C)(C)O] |
| 115 | 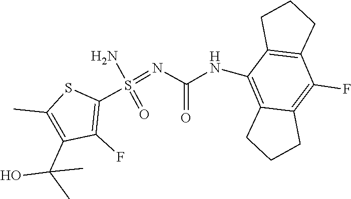 [SMILES: Cc1sc(S(N)(=O)=NC(=O)Nc2c3c(c(F)c4c2CCC4)CCC3)c(F)c1C(C)(C)O] |
| 116 | 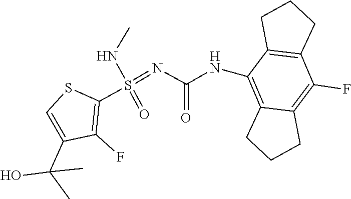 [SMILES: CNS(=O)(=NC(=O)Nc1c2c(c(F)c3c1CCC3)CCC2)c1scc(C(C)(C)O)c1F] |
| 116a | 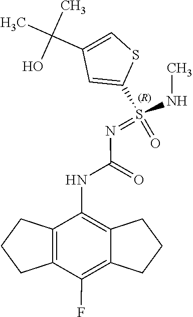 |
| 116b | 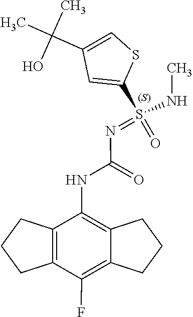 |
| 117 | 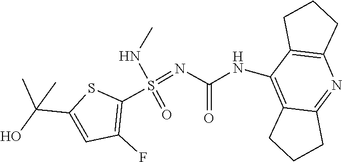 [SMILES: CNS(=O)(=NC(=O)Nc1c2c(nc3c1CCC3)CCC2)c1sc(C(C)(C)O)cc1F] |
| 118 | 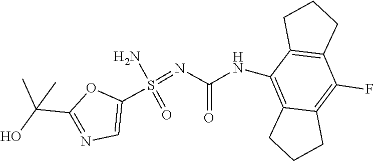 [SMILES: CC(C)(O)c1ncc(S(N)(=O)=NC(=O)Nc2c3c(c(F)c4c2CCC4)CCC3)o1] |
| 119 | 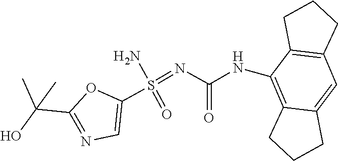 [SMILES: CC(C)(O)c1ncc(S(N)(=O)=NC(=O)Nc2c3c(cc4c2CCC4)CCC3)o1] |
| 120 | 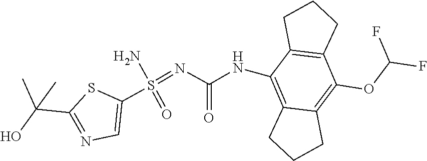 [SMILES: CC(C)(O)c1ncc(S(N)(=O)=NC(=O)Nc2c3c(c(OC(F)F)c4c2CCC4)CCC3)s1] |
| 120a | 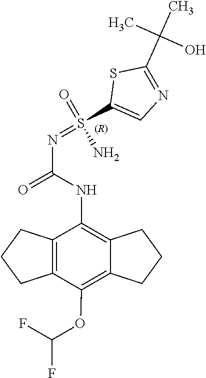 |
| 120b | 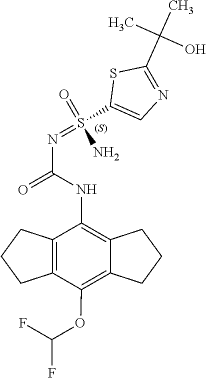 |
| 121 | 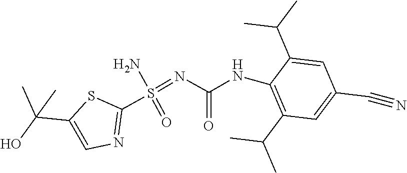 [SMILES: CC(C)c1cc(C#N)cc(C(C)C)c1NC(=O)N=S(N)(=O)c1ncc(C(C)(C)O)s1] |
| 121a | 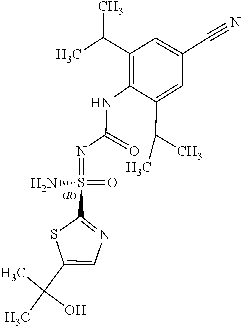 |
| 121b | 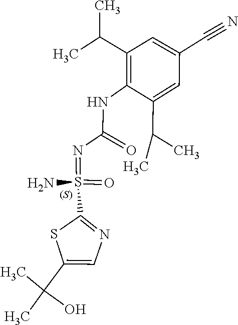 |
| 122 | 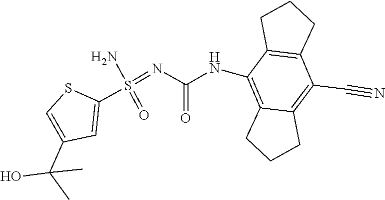 [SMILES: CC(C)(O)c1csc(S(N)(=O)=NC(=O)Nc2c3c(c(C#N)c4c2CCC4)CCC3)c1] |
| 122a | 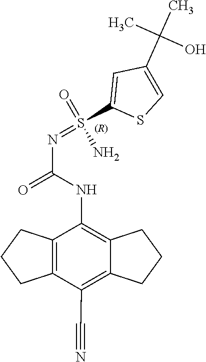 |
| 122b | 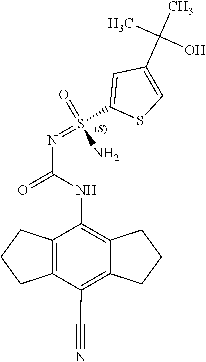 |
| 123 | 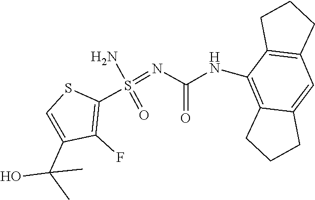 [SMILES: CC(C)(O)c1csc(S(N)(=O)=NC(=O)Nc2c3c(cc4c2CCC4)CCC3)c1F] |
| 124 | 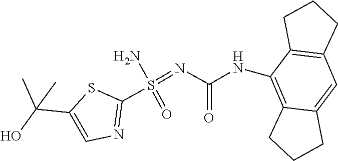 [SMILES: CC(C)(O)c1cnc(S(N)(=O)=NC(=O)Nc2c3c(cc4c2CCC4)CCC3)s1] |
| 125 | 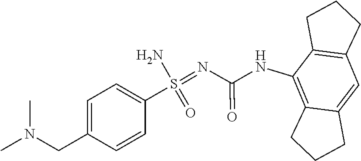 [SMILES: CN(C)Cc1ccc(S(N)(=O)=NC(=O)Nc2c3c(cc4c2CCC4)CCC3)cc1] |
| 125a | 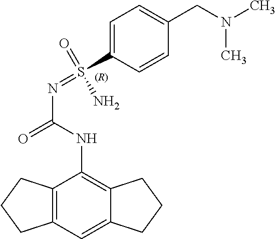 |
| 125b | 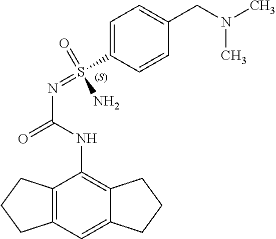 |
| 126 | 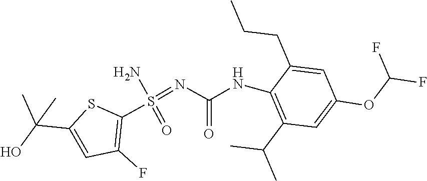 [SMILES: CCCc1cc(OC(F)F)cc(C(C)C)c1NC(=O)N=S(N)(=O)c1sc(C(C)(C)O)cc1F] |
| 127 | 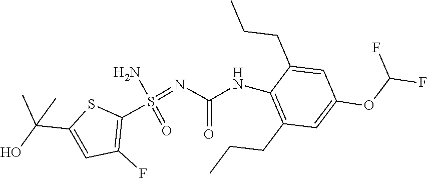 [SMILES: CCCc1cc(OC(F)F)cc(CCC)c1NC(=O)N=S(N)(=O)c1sc(C(C)(C)O)cc1F] |
| 128 | 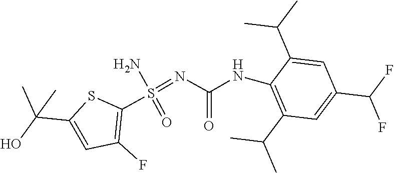 [SMILES: CC(C)c1cc(C(F)F)cc(C(C)C)c1NC(=O)N=S(N)(=O)c1sc(C(C)(C)O)cc1F] |
| 129 | 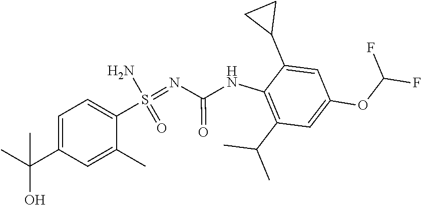 [SMILES: Cc1cc(C(C)(C)O)ccc1S(N)(=O)=NC(=O)Nc1c(C(C)C)cc(OC(F)F)cc1C1CC1] |
| 129a | 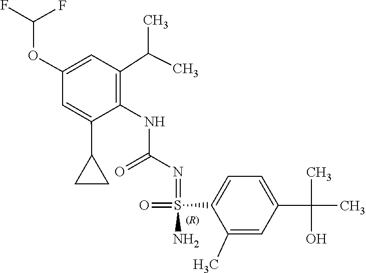 |
| 129b | 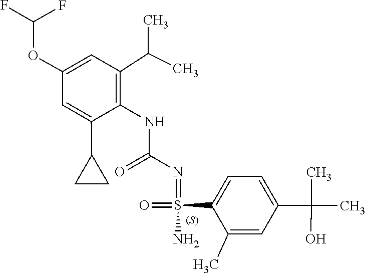 |
| 130 | 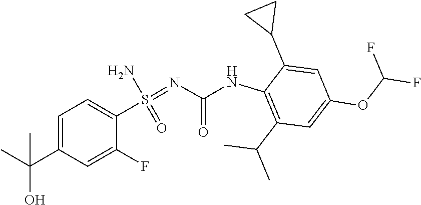 [SMILES: CC(C)c1cc(OC(F)F)cc(C2CC2)c1NC(=O)N=S(N)(=O)c1ccc(C(C)(C)O)cc1F] |
| 130a | 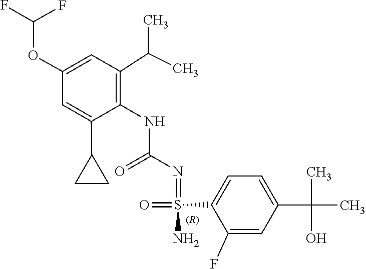 |
| 130b | 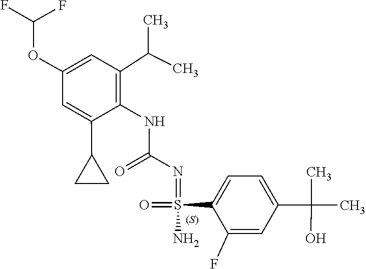 |
| 131 | 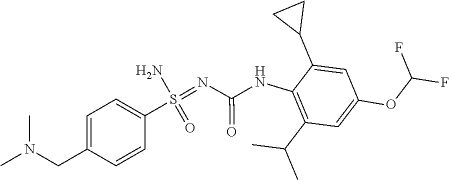 [SMILES: CC(C)c1cc(OC(F)F)cc(C2CC2)c1NC(=O)N=S(N)(=O)c1ccc(CN(C)C)cc1] |
| 131a | 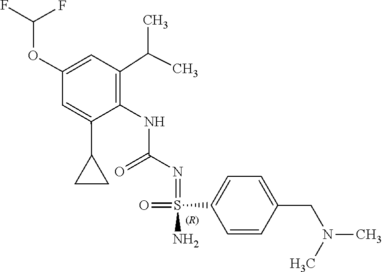 |
| 131b | 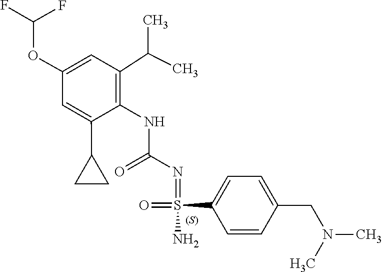 |
| 132 | 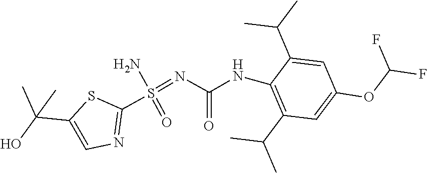 [SMILES: CC(C)c1cc(OC(F)F)cc(C(C)C)c1NC(=O)N=S(N)(=O)c1ncc(C(C)(C)O)s1] |
| 133 | 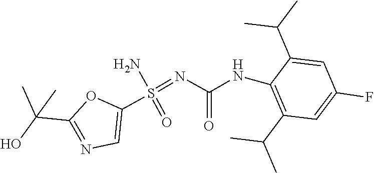 [SMILES: CC(C)c1cc(F)cc(C(C)C)c1NC(=O)N=S(N)(=O)c1cnc(C(C)(C)O)o1] |
| 134 | 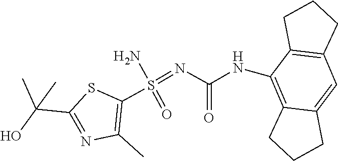 [SMILES: Cc1nc(C(C)(C)O)sc1S(N)(=O)=NC(=O)Nc1c2c(cc3c1CCC3)CCC2] |
| 134a | 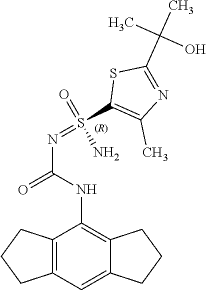 |
| 134b | 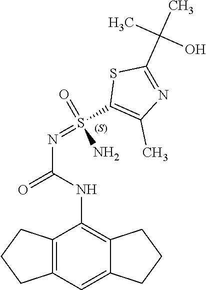 |
| 135 | 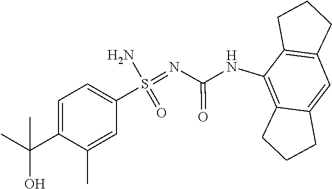 [SMILES: Cc1cc(S(N)(=O)=NC(=O)Nc2c3c(cc4c2CCC4)CCC3)ccc1C(C)(C)O] |
| 135a | 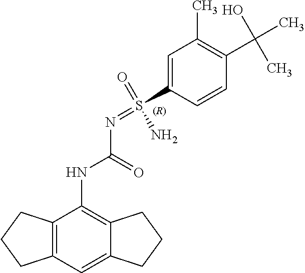 |
| 135b | 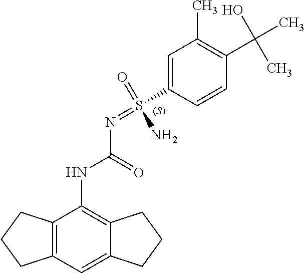 |
| 136 | 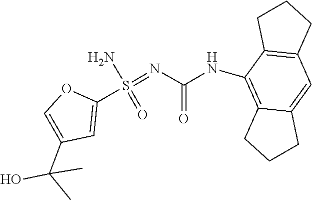 [SMILES: CC(C)(O)c1coc(S(N)(=O)=NC(=O)Nc2c3c(cc4c2CCC4)CCC3)c1] |
| 136a | 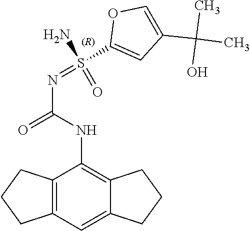 |
| 136b | 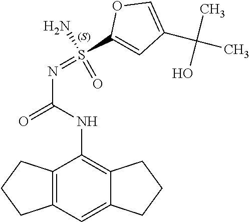 |
| 137 | 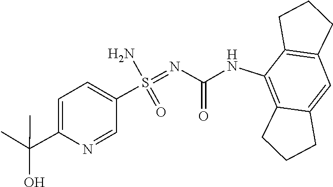 [SMILES: CC(C)(O)c1ccc(S(N)(=O)=NC(=O)Nc2c3c(cc4c2CCC4)CCC3)cn1] |
| 137a | 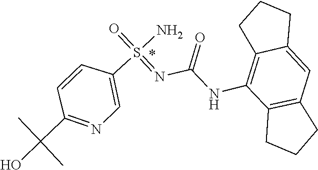 [SMILES: CC(C)(O)c1ccc(S(N)(=O)=NC(=O)Nc2c3c(cc4c2CCC4)CCC3)cn1] |
| 137b | 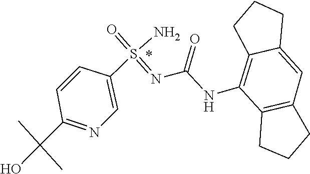 [SMILES: CC(C)(O)c1ccc(S(N)(=O)=NC(=O)Nc2c3c(cc4c2CCC4)CCC3)cn1] |
| 138b | 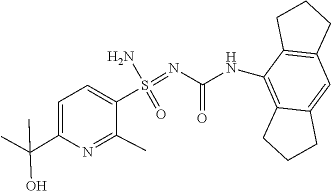 [SMILES: Cc1nc(C(C)(C)O)ccc1S(N)(=O)=NC(=O)Nc1c2c(cc3c1CCC3)CCC2] |
| 138a | 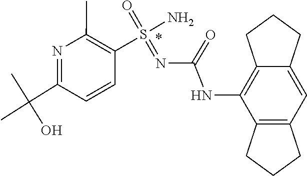 [SMILES: Cc1nc(C(C)(C)O)ccc1S(N)(=O)=NC(=O)Nc1c2c(cc3c1CCC3)CCC2] |
| 138b | 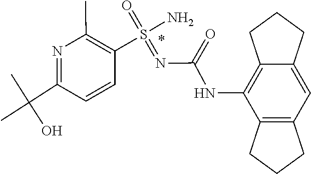 [SMILES: Cc1nc(C(C)(C)O)ccc1S(N)(=O)=NC(=O)Nc1c2c(cc3c1CCC3)CCC2] |
| 139 | 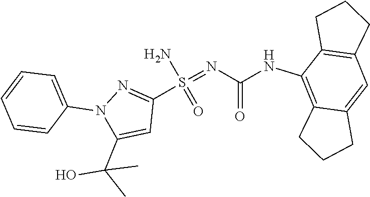 [SMILES: CC(C)(O)c1cc(S(N)(=O)=NC(=O)Nc2c3c(cc4c2CCC4)CCC3)nn1-c1ccccc1] |
| 139a | 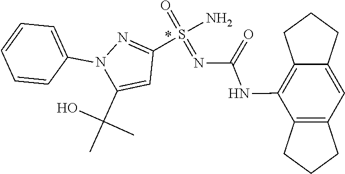 [SMILES: CC(C)(O)c1cc(S(N)(=O)=NC(=O)Nc2c3c(cc4c2CCC4)CCC3)nn1-c1ccccc1] |
| 139b | 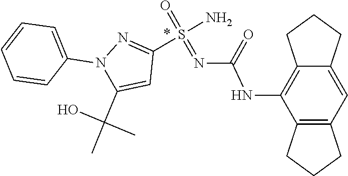 [SMILES: CC(C)(O)c1cc(S(N)(=O)=NC(=O)Nc2c3c(cc4c2CCC4)CCC3)nn1-c1ccccc1] |
| 140 | 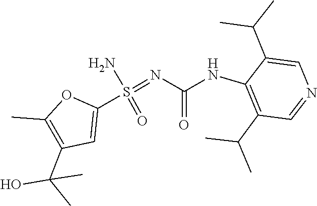 [SMILES: Cc1oc(S(N)(=O)=NC(=O)Nc2c(C(C)C)cncc2C(C)C)cc1C(C)(C)O] |
| 141 | 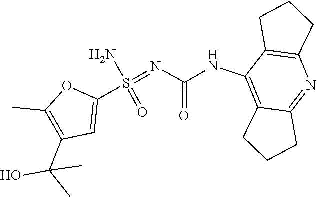 [SMILES: Cc1oc(S(N)(=O)=NC(=O)Nc2c3c(nc4c2CCC4)CCC3)cc1C(C)(C)O] |
| 142 | 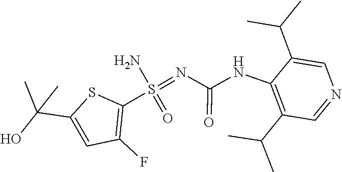 [SMILES: CC(C)c1cncc(C(C)C)c1NC(=O)N=S(N)(=O)c1sc(C(C)(C)O)cc1F] |
| 143 | 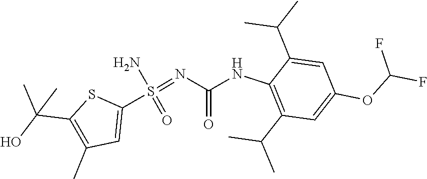 [SMILES: Cc1cc(S(N)(=O)=NC(=O)Nc2c(C(C)C)cc(OC(F)F)cc2C(C)C)sc1C(C)(C)O] |
| 143a | 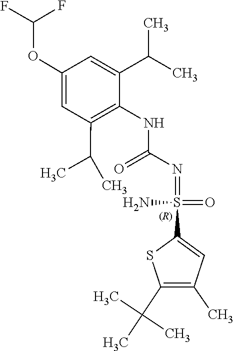 |
| 143b | 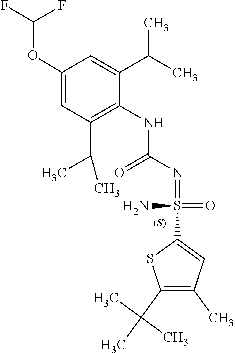 |
| 144 | 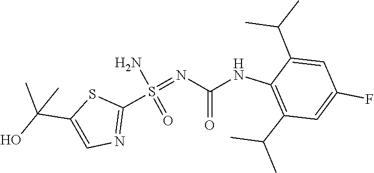 [SMILES: CC(C)c1cc(F)cc(C(C)C)c1NC(=O)N=S(N)(=O)c1ncc(C(C)(C)O)s1] |
| 144a | 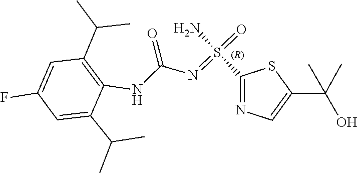 |
| 144b | 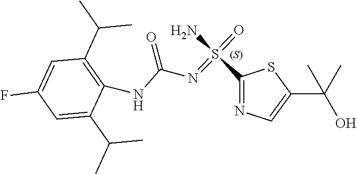 |
| 145 | 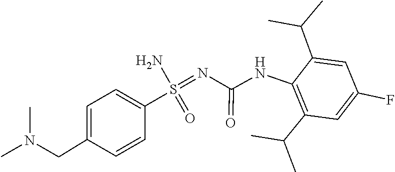 [SMILES: CC(C)c1cc(F)cc(C(C)C)c1NC(=O)N=S(N)(=O)c1ccc(CN(C)C)cc1] |
| 145a | 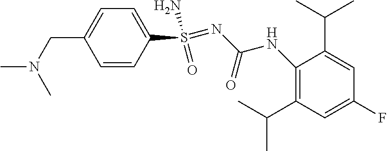 [SMILES: CC(C)c1cc(F)cc(C(C)C)c1NC(=O)N=[S@@](N)(=O)c1ccc(CN(C)C)cc1] |
| 145b | 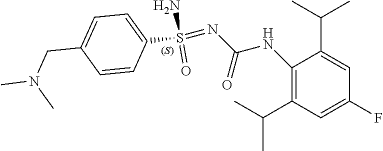 |
| 146 | 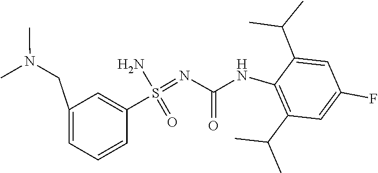 [SMILES: CC(C)c1cc(F)cc(C(C)C)c1NC(=O)N=S(N)(=O)c1cccc(CN(C)C)c1] |
| 147 | 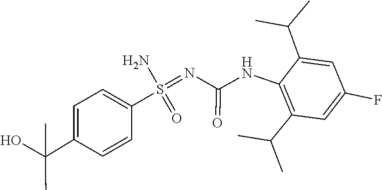 [SMILES: CC(C)c1cc(F)cc(C(C)C)c1NC(=O)N=S(N)(=O)c1ccc(C(C)(C)O)cc1] |
| 148 | 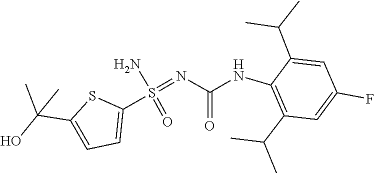 [SMILES: CC(C)c1cc(F)cc(C(C)C)c1NC(=O)N=S(N)(=O)c1ccc(C(C)(C)O)s1] |
| 148a | 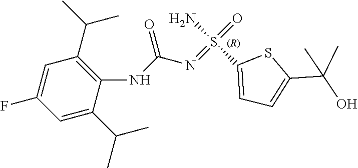 |
| 148b | 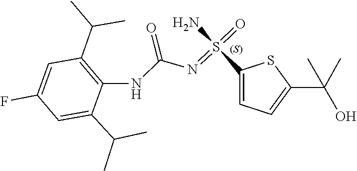 |
| 149 | 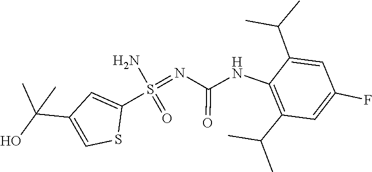 [SMILES: CC(C)c1cc(F)cc(C(C)C)c1NC(=O)N=S(N)(=O)c1cc(C(C)(C)O)cs1] |
| 149a | 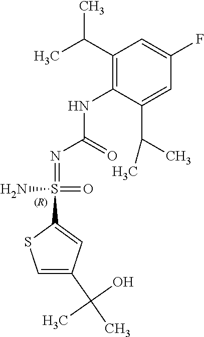 |
| 149b | 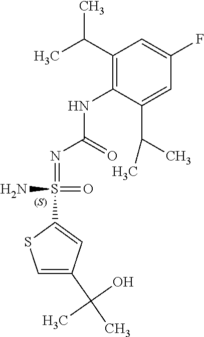 |
| 150 | 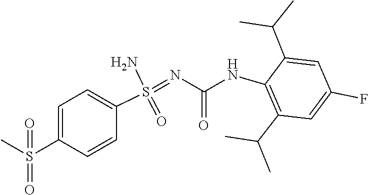 [SMILES: CC(C)c1cc(F)cc(C(C)C)c1NC(=O)N=S(N)(=O)c1ccc(S(C)(=O)=O)cc1] |
| 151a' | 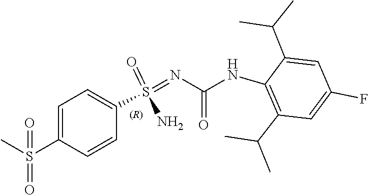 |
| 151b' | 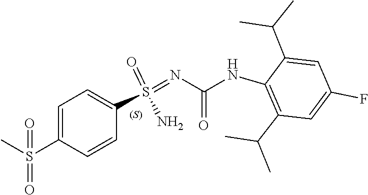 |
| 151 | 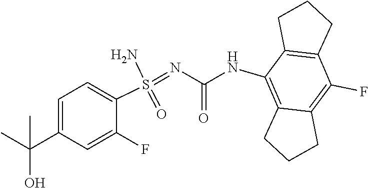 [SMILES: CC(C)(O)c1ccc(S(N)(=O)=NC(=O)Nc2c3c(c(F)c4c2CCC4)CCC3)c(F)c1] |
| 151a | 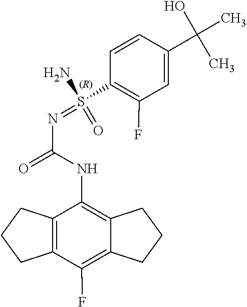 |
| 151b | 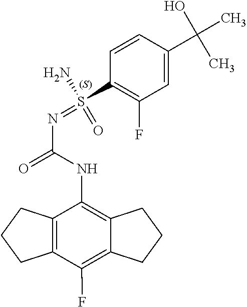 |
| 152 | 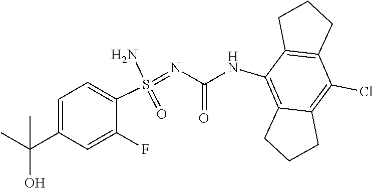 [SMILES: CC(C)(O)c1ccc(S(N)(=O)=NC(=O)Nc2c3c(c(Cl)c4c2CCC4)CCC3)c(F)c1] |
| 152a | 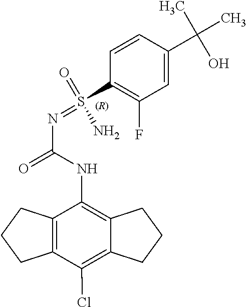 |
| 152b | 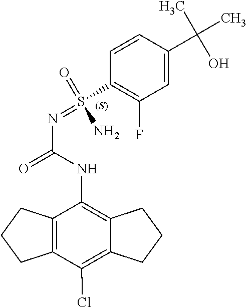 |
| 153 | 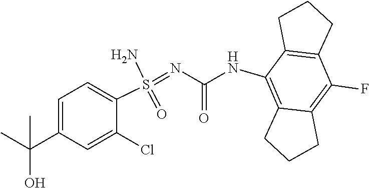 [SMILES: CC(C)(O)c1ccc(S(N)(=O)=NC(=O)Nc2c3c(c(F)c4c2CCC4)CCC3)c(Cl)c1] |
| 153a | 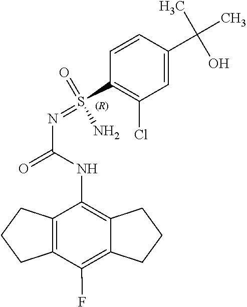 |
| 153b | 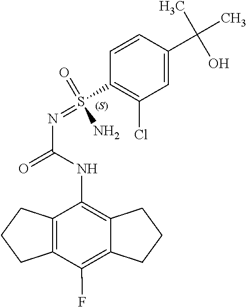 |
| 154 | 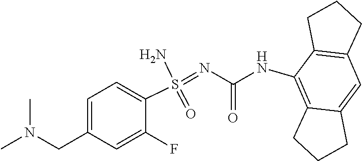 [SMILES: CN(C)Cc1ccc(S(N)(=O)=NC(=O)Nc2c3c(cc4c2CCC4)CCC3)c(F)c1] |
| 154a | 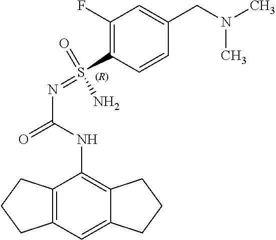 |
| 154b | 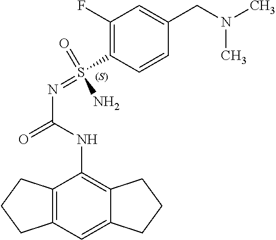 |
| 155 | 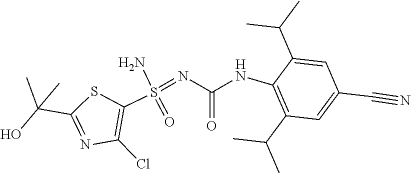 [SMILES: CC(C)c1cc(C#N)cc(C(C)C)c1NC(=O)N=S(N)(=O)c1sc(C(C)(C)O)nc1Cl] |
| 156 | 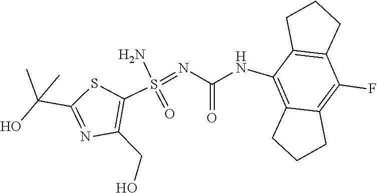 [SMILES: CC(C)(O)c1nc(CO)c(S(N)(=O)=NC(=O)Nc2c3c(c(F)c4c2CCC4)CCC3)s1] |
| 157 | 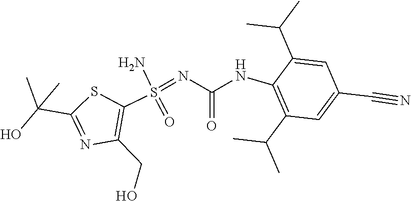 [SMILES: CC(C)c1cc(C#N)cc(C(C)C)c1NC(=O)N=S(N)(=O)c1sc(C(C)(C)O)nc1CO] |
| 157a | 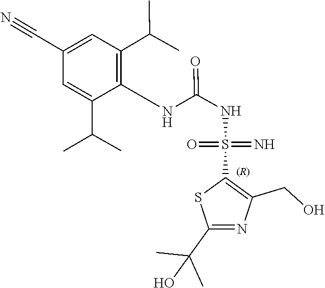 |
| 157b | 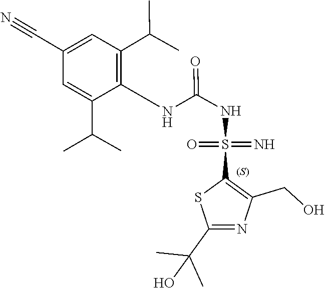 |
| 158 | 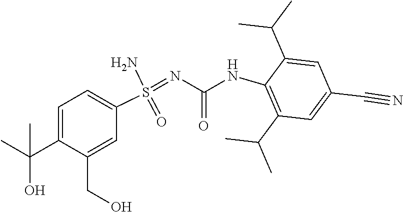 [SMILES: CC(C)c1cc(C#N)cc(C(C)C)c1NC(=O)N=S(N)(=O)c1ccc(C(C)(C)O)c(CO)c1] |
| 158a | 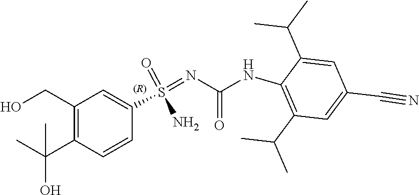 |
| 158b | 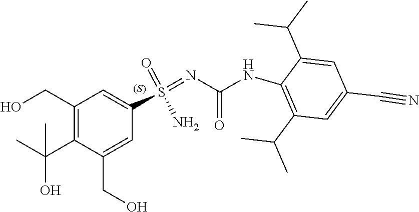 |
| 159 | 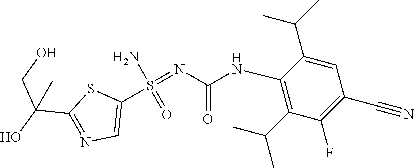 [SMILES: CC(C)c1cc(C#N)c(F)c(C(C)C)c1NC(=O)N=S(N)(=O)c1cnc(C(C)(O)CO)s1] |
| 159a | 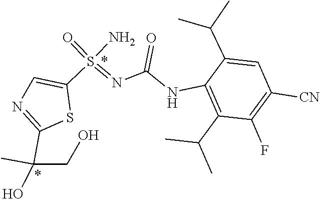 [SMILES: CC(C)c1cc(C#N)c(F)c(C(C)C)c1NC(=O)N=S(N)(=O)c1cnc(C(C)(O)CO)s1] |
| 159ba | 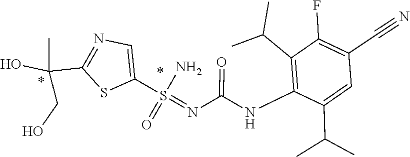 [SMILES: CC(C)c1cc(C#N)c(F)c(C(C)C)c1NC(=O)N=S(N)(=O)c1cnc(C(C)(O)CO)s1] |
| 159ab | 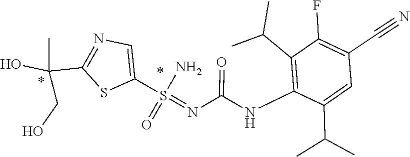 [SMILES: CC(C)c1cc(C#N)c(F)c(C(C)C)c1NC(=O)N=S(N)(=O)c1cnc(C(C)(O)CO)s1] |
| 160 | 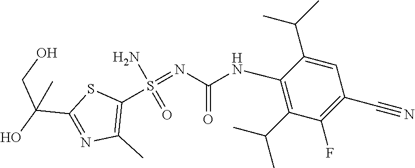 [SMILES: Cc1nc(C(C)(O)CO)sc1S(N)(=O)=NC(=O)Nc1c(C(C)C)cc(C#N)c(F)c1C(C)C] |
| 161 | 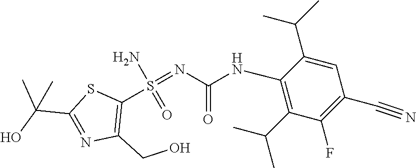 [SMILES: CC(C)c1cc(C#N)c(F)c(C(C)C)c1NC(=O)N=S(N)(=O)c1sc(C(C)(C)O)nc1CO] |
| 161a | 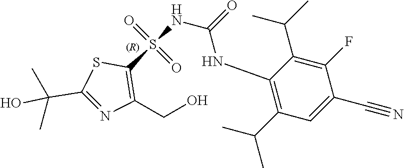 |
| 161b | 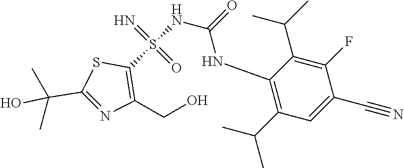 [SMILES: CC(C)c1cc(C#N)c(F)c(C(C)C)c1NC(=O)N[S@](=N)(=O)c1sc(C(C)(C)O)nc1CO] |
| 162 | 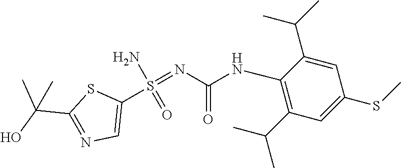 [SMILES: CSc1cc(C(C)C)c(NC(=O)N=S(N)(=O)c2cnc(C(C)(C)O)s2)c(C(C)C)c1] |
| 163 | 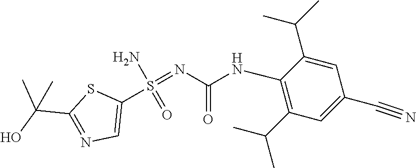 [SMILES: CC(C)c1cc(C#N)cc(C(C)C)c1NC(=O)N=S(N)(=O)c1cnc(C(C)(C)O)s1] |
| 164 | 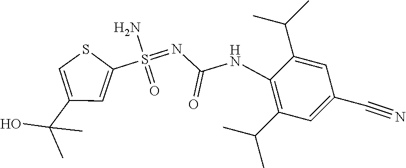 [SMILES: CC(C)c1cc(C#N)cc(C(C)C)c1NC(=O)N=S(N)(=O)c1cc(C(C)(C)O)cs1] |
| 165 | 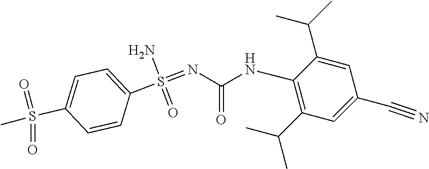 [SMILES: CC(C)c1cc(C#N)cc(C(C)C)c1NC(=O)N=S(N)(=O)c1ccc(S(C)(=O)=O)cc1] |
| 165a | 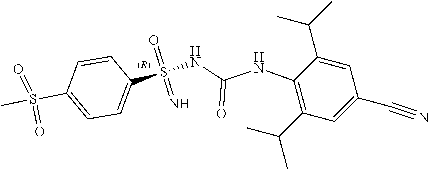 |
| 165b | 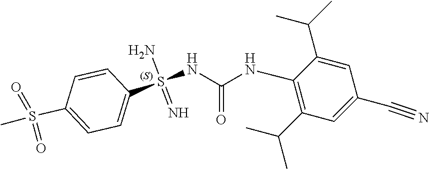 |
| 166 | 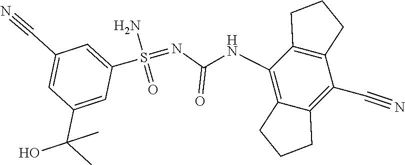 [SMILES: CC(C)(O)c1cc(C#N)cc(S(N)(=O)=NC(=O)Nc2c3c(c(C#N)c4c2CCC4)CCC3)c1] |
| 167 | 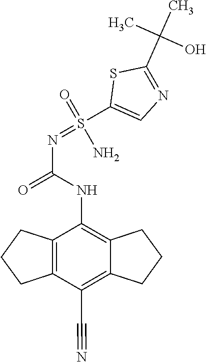 [SMILES: CC(C)(O)c1ncc(S(N)(=O)=NC(=O)Nc2c3c(c(C#N)c4c2CCC4)CCC3)s1] |
| 167a | 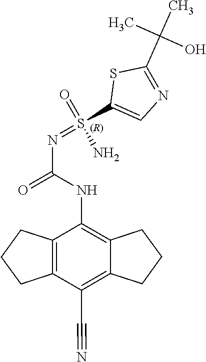 |
| 167b | 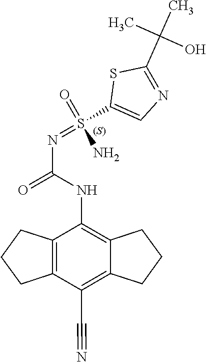 |
| 168 | 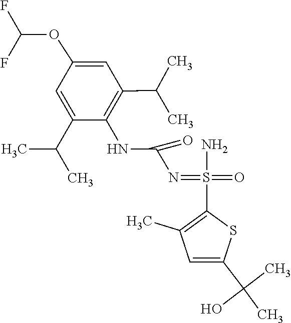 [SMILES: Cc1cc(C(C)(C)O)sc1S(N)(=O)=NC(=O)Nc1c(C(C)C)cc(OC(F)F)cc1C(C)C] |
| 168a | 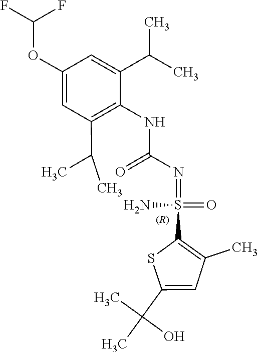 |
| 168b | 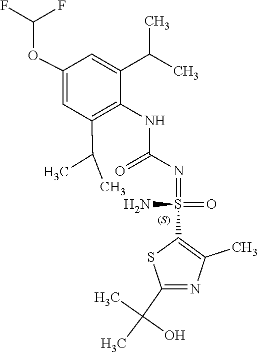 |
| 170 | 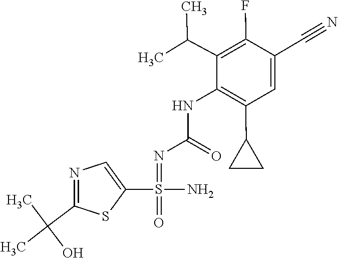 [SMILES: CC(C)c1c(F)c(C#N)cc(C2CC2)c1NC(=O)N=S(N)(=O)c1cnc(C(C)(C)O)s1] |
| 170a | 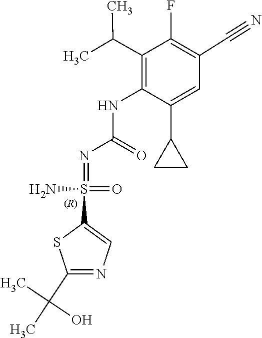 |
| 170b | 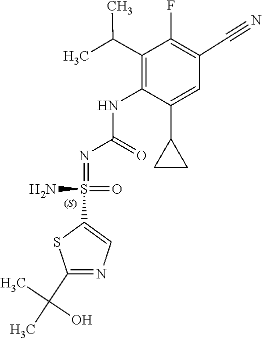 |
| 171 | 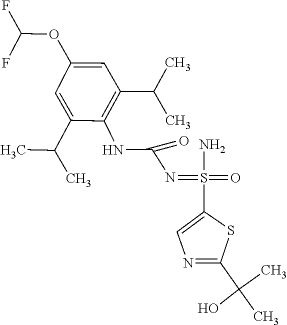 [SMILES: CC(C)c1cc(OC(F)F)cc(C(C)C)c1NC(=O)N=S(N)(=O)c1cnc(C(C)(C)O)s1] |
| 171a | 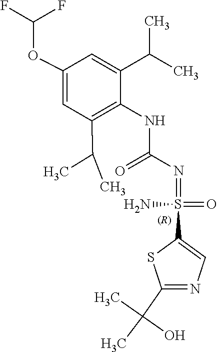 |
| 171b | 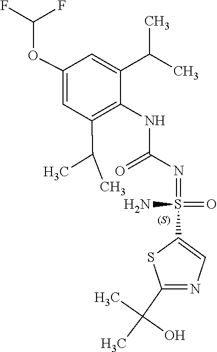 |
| 172 | 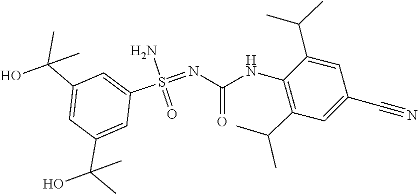 [SMILES: CC(C)c1cc(C#N)cc(C(C)C)c1NC(=O)N=S(N)(=O)c1cc(C(C)(C)O)cc(C(C)(C)O)c1] |
| 172a | 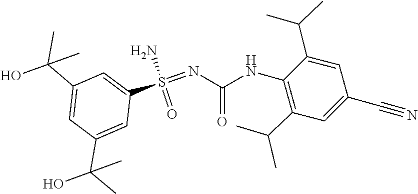 [SMILES: CC(C)c1cc(C#N)cc(C(C)C)c1NC(=O)N=[S@@](N)(=O)c1cc(C(C)(C)O)cc(C(C)(C)O)c1] |
| 172b | 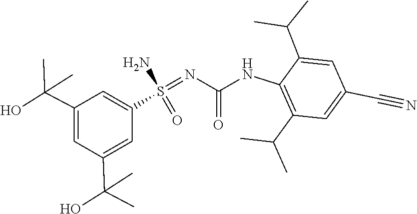 [SMILES: CC(C)c1cc(C#N)cc(C(C)C)c1NC(=O)N=[S@](N)(=O)c1cc(C(C)(C)O)cc(C(C)(C)O)c1] |
| 173 | 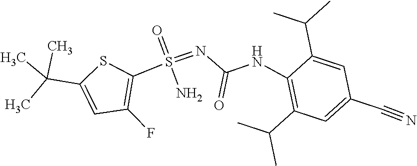 |
| 173a | 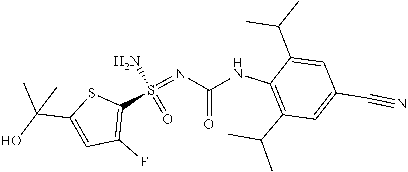 [SMILES: CC(C)c1cc(C#N)cc(C(C)C)c1NC(=O)N=[S@@](N)(=O)c1sc(C(C)(C)O)cc1F] |
| 173b | 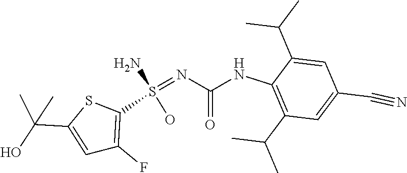 |
| 174 | 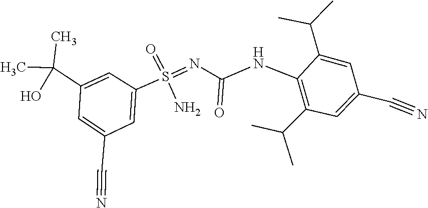 [SMILES: CC(C)c1cc(C#N)cc(C(C)C)c1NC(=O)N=S(N)(=O)c1cc(C#N)cc(C(C)(C)O)c1] |
| 174a | 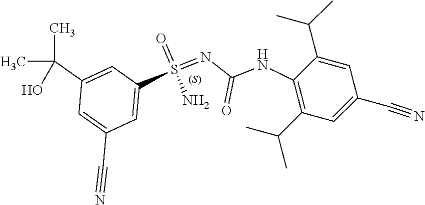 |
| 174b | 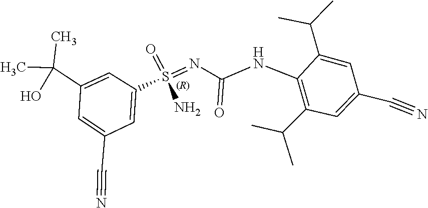 |
| 176 | 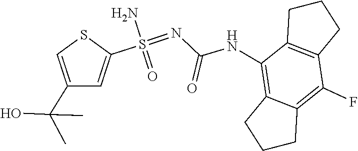 [SMILES: CC(C)(O)c1csc(S(N)(=O)=NC(=O)Nc2c3c(c(F)c4c2CCC4)CCC3)c1] |
| 176a | 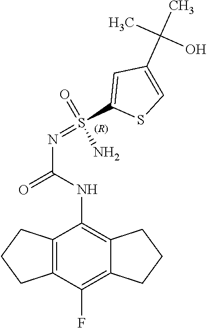 |
| 176b | 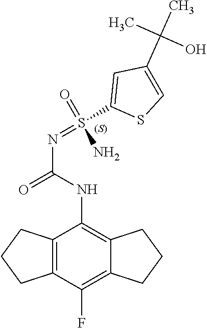 |
| 177 | 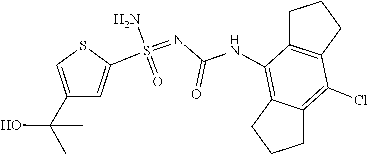 |
| 177a | 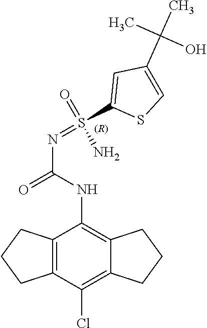 |
| 177b | 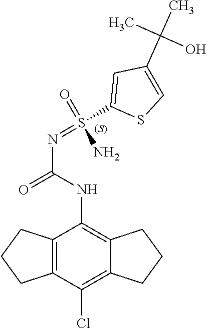 |
| 178 | 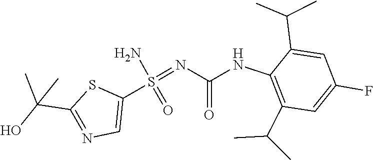 [SMILES: CC(C)c1cc(F)cc(C(C)C)c1NC(=O)N=S(N)(=O)c1cnc(C(C)(C)O)s1] |
| 178a | 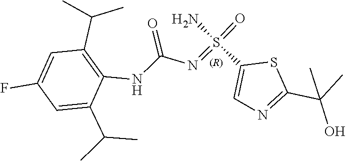 |
| 178b | 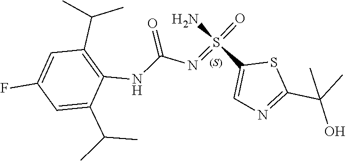 |
| 179 | 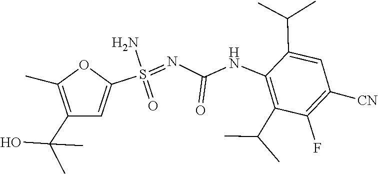 [SMILES: Cc1oc(S(N)(=O)=NC(=O)Nc2c(C(C)C)cc(C#N)c(F)c2C(C)C)cc1C(C)(C)O] |
| 179a | 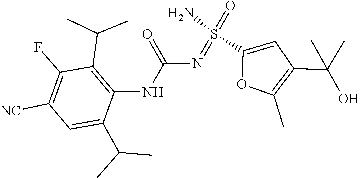 [SMILES: Cc1oc([S@](N)(=O)=NC(=O)Nc2c(C(C)C)cc(C#N)c(F)c2C(C)C)cc1C(C)(C)O] |
| 179b | 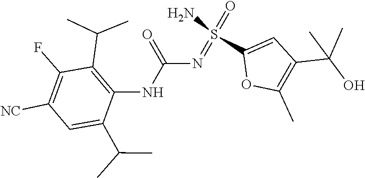 [SMILES: Cc1oc([S@@](N)(=O)=NC(=O)Nc2c(C(C)C)cc(C#N)c(F)c2C(C)C)cc1C(C)(C)O] |
| 180 | 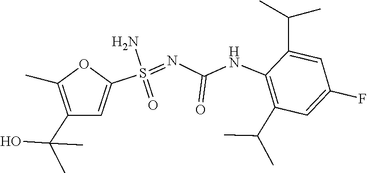 [SMILES: Cc1oc(S(N)(=O)=NC(=O)Nc2c(C(C)C)cc(F)cc2C(C)C)cc1C(C)(C)O] |
| 180a | 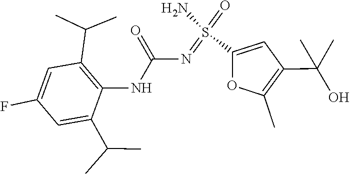 [SMILES: Cc1oc([S@](N)(=O)=NC(=O)Nc2c(C(C)C)cc(F)cc2C(C)C)cc1C(C)(C)O] |
| 180b | 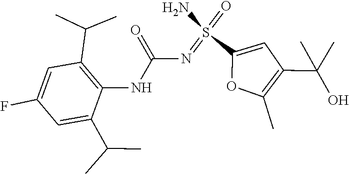 [SMILES: Cc1oc([S@@](N)(=O)=NC(=O)Nc2c(C(C)C)cc(F)cc2C(C)C)cc1C(C)(C)O] |
| 181 | 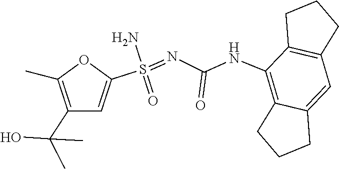 [SMILES: Cc1oc(S(N)(=O)=NC(=O)Nc2c3c(cc4c2CCC4)CCC3)cc1C(C)(C)O] |
| 181a | 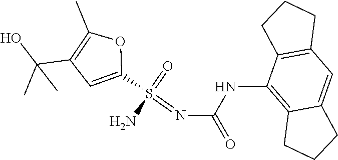 [SMILES: Cc1oc([S@](N)(=O)=NC(=O)Nc2c3c(cc4c2CCC4)CCC3)cc1C(C)(C)O] |
| 181b | 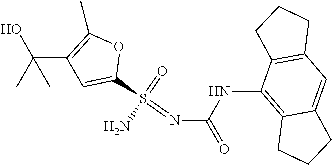 [SMILES: Cc1oc([S@@](N)(=O)=NC(=O)Nc2c3c(cc4c2CCC4)CCC3)cc1C(C)(C)O] |
| 182 | 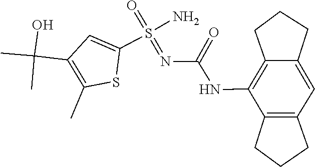 [SMILES: Cc1sc(S(N)(=O)=NC(=O)Nc2c3c(cc4c2CCC4)CCC3)cc1C(C)(C)O] |
| 182a | 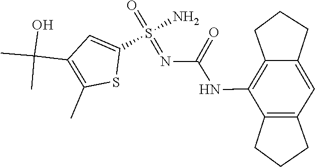 [SMILES: Cc1sc([S@@](N)(=O)=NC(=O)Nc2c3c(cc4c2CCC4)CCC3)cc1C(C)(C)O] |
| 182b | 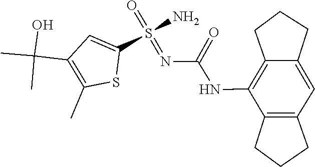 [SMILES: Cc1sc([S@](N)(=O)=NC(=O)Nc2c3c(cc4c2CCC4)CCC3)cc1C(C)(C)O] |
| 183 | 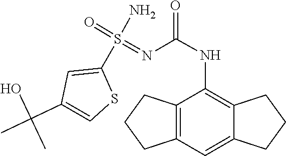 [SMILES: CC(C)(O)c1csc(S(N)(=O)=NC(=O)Nc2c3c(cc4c2CCC4)CCC3)c1] |
| 183a | 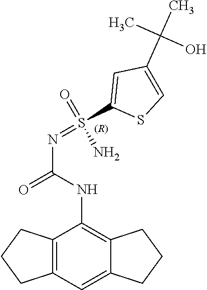 |
| 183b | 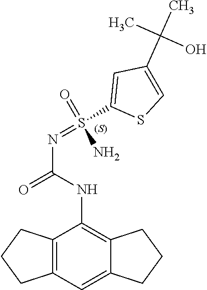 |
| 184 | 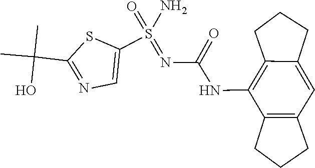 [SMILES: CC(C)(O)c1ncc(S(N)(=O)=NC(=O)Nc2c3c(cc4c2CCC4)CCC3)s1] |
| 185 | 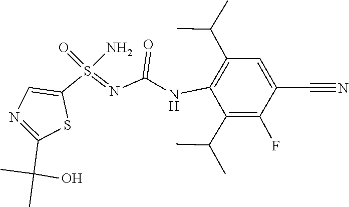 [SMILES: CC(C)c1cc(C#N)c(F)c(C(C)C)c1NC(=O)N=S(N)(=O)c1cnc(C(C)(C)O)s1] |
| 185a | 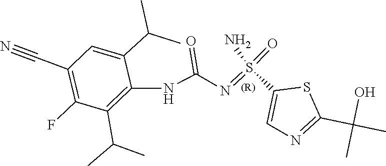 |
| 185b | 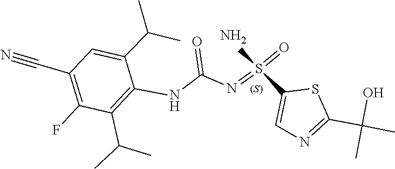 |
| 186 | 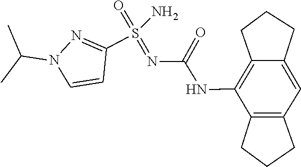 [SMILES: CC(C)n1ccc(S(N)(=O)=NC(=O)Nc2c3c(cc4c2CCC4)CCC3)n1] |
| 186a | 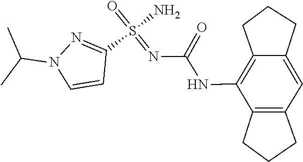 [SMILES: CC(C)n1ccc([S@@](N)(=O)=NC(=O)Nc2c3c(cc4c2CCC4)CCC3)n1] |
| 186b | 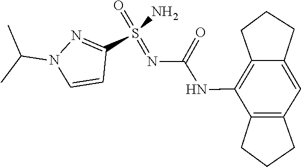 [SMILES: CC(C)n1ccc([S@](N)(=O)=NC(=O)Nc2c3c(cc4c2CCC4)CCC3)n1] |
| 187 | 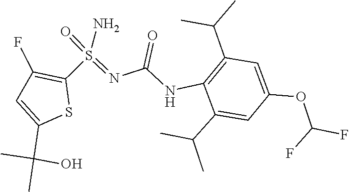 [SMILES: CC(C)c1cc(OC(F)F)cc(C(C)C)c1NC(=O)N=S(N)(=O)c1sc(C(C)(C)O)cc1F] |
| 187a | 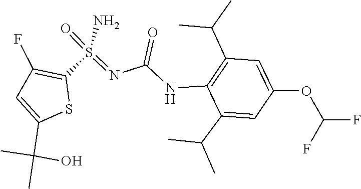 [SMILES: CC(C)c1cc(OC(F)F)cc(C(C)C)c1NC(=O)N=[S@](N)(=O)c1sc(C(C)(C)O)cc1F] |
| 187b | 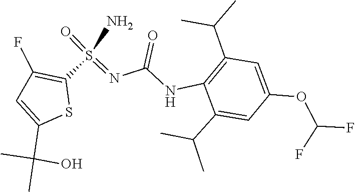 [SMILES: CC(C)c1cc(OC(F)F)cc(C(C)C)c1NC(=O)N=[S@](N)(=O)c1sc(C(C)(C)O)cc1F] |
| 188 | 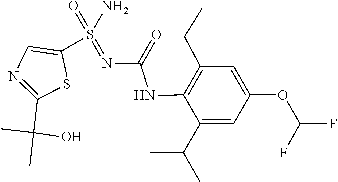 [SMILES: CCc1cc(OC(F)F)cc(C(C)C)c1NC(=O)N=S(N)(=O)c1cnc(C(C)(C)O)s1] |
| 188a | 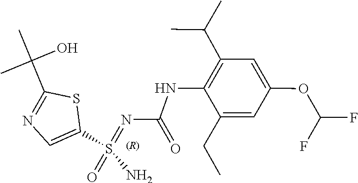 |
| 188b | 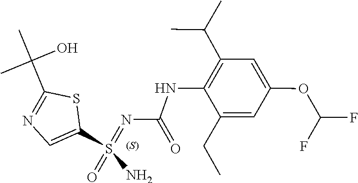 |
| 189 | 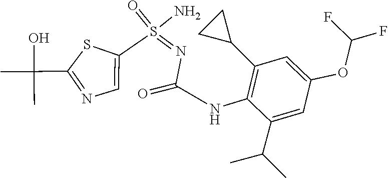 [SMILES: CC(C)c1cc(OC(F)F)cc(C2CC2)c1NC(=O)N=S(N)(=O)c1cnc(C(C)(C)O)s1] |
| 189a | 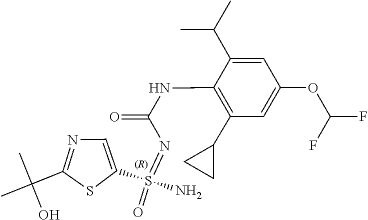 |
| 189b | 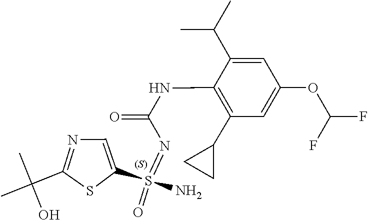 |

and pharmaceutically acceptable salts thereof.

In another aspect, an NLRP3 antagonist is a compound selected from the group consisting of the compounds in Table 12 below:

TABLE 12

| | |
|---|---|
| 190 | 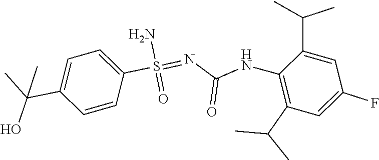 |
| 190a | 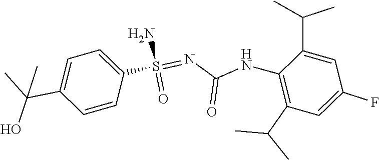 |
| 190b | 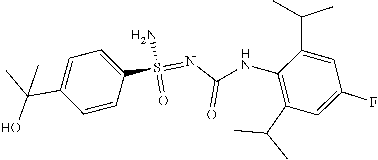 |
| 191 | 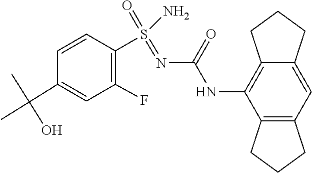 [SMILES: CC(C)(O)c1ccc(S(N)(=O)=NC(=O)Nc2c3c(cc4c2CCC4)CCC3)c(F)c1] |
| 191a | 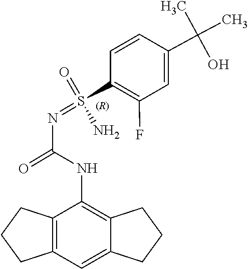 |
| 191b | 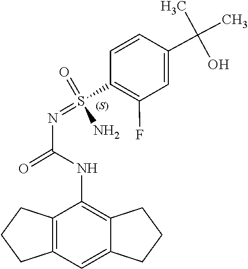 |
| 192 | 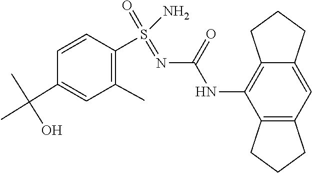 [SMILES: Cc1cc(C(C)(C)O)ccc1S(N)(=O)=NC(=O)Nc1c2c(cc3c1CCC3)CCC2] |
| 192a | 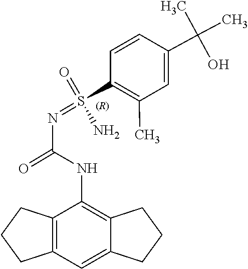 |
| 192b | 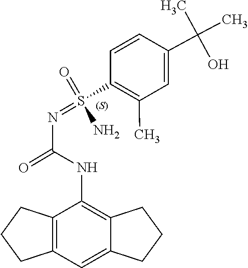 |
| 193 | 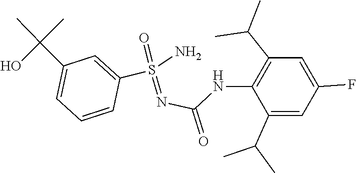 [SMILES: CC(C)c1cc(F)cc(C(C)C)c1NC(=O)N=S(N)(=O)c1cccc(C(C)(C)O)c1] |
| 193a | 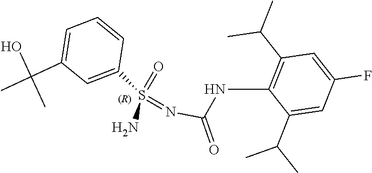 |
| 193b | 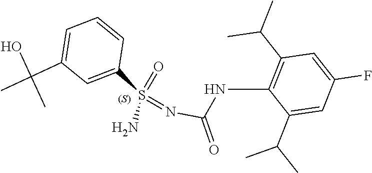 |
| 194 | 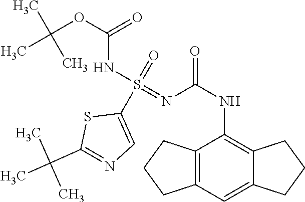 [SMILES: CC(C)(C)OC(=O)NS(=O)(=NC(=O)Nc1c2c(cc3c1CCC3)CCC2)c1cnc(C(C)(C)C)s1] |
| 195 | 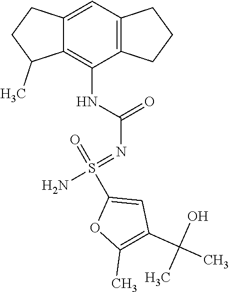 [SMILES: Cc1oc(S(N)(=O)=NC(=O)Nc2c3c(cc4c2C(C)CC4)CCC3)cc1C(C)(C)O] |
| 195a | 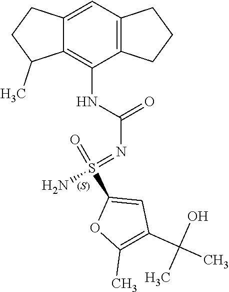 |
| 195ba | 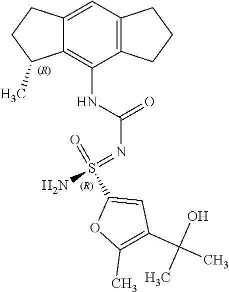 |
| 195bb | 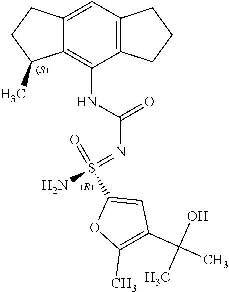 |
| 195e | 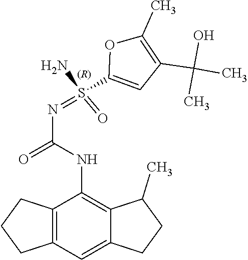 |
| 196 | 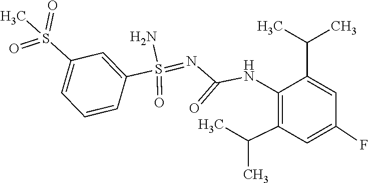 [SMILES: CC(C)c1cc(F)cc(C(C)C)c1NC(=O)N=S(N)(=O)c1cccc(S(C)(=O)=O)c1] |
| 197 | 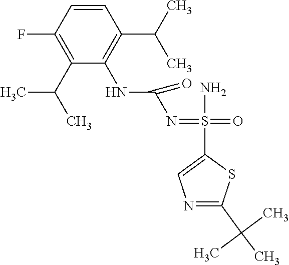 [SMILES: CC(C)c1ccc(F)c(C(C)C)c1NC(=O)N=S(N)(=O)c1cnc(C(C)(C)C)s1] |
| 198 | 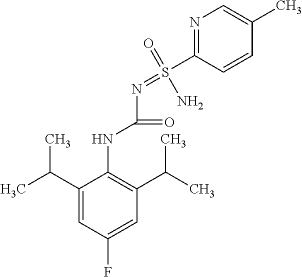 [SMILES: Cc1ccc(S(N)(=O)=NC(=O)Nc2c(C(C)C)cc(F)cc2C(C)C)nc1] |
| 199 | |
| 200 | |
| 201 | |
| 202 | 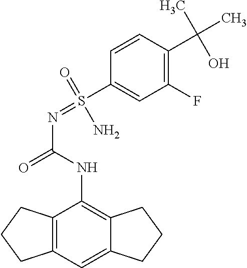 [SMILES: CC(C)(O)c1ccc(S(N)(=O)=NC(=O)Nc2c3c(cc4c2CCC4)CCC3)cc1F] |
| 202a | 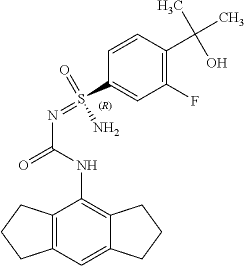 |
| 202b | 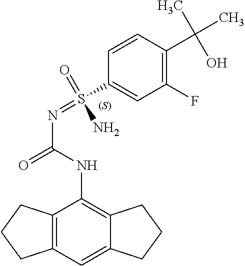 |
| 203 | 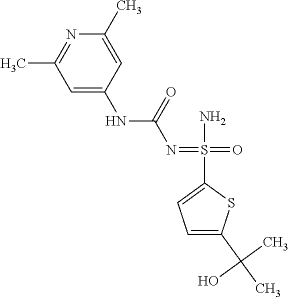 [SMILES: Cc1cc(NC(=O)N=S(N)(=O)c2ccc(C(C)(C)O)s2)cc(C)n1] |
| 204 | 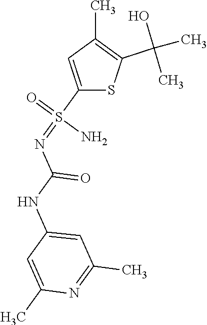 [SMILES: Cc1cc(NC(=O)N=S(N)(=O)c2cc(C)c(C(C)(C)O)s2)cc(C)n1] |
| 205 | 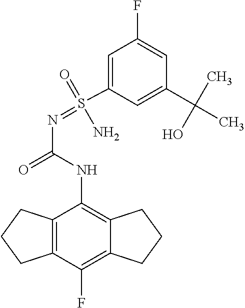 [SMILES: CC(C)(O)c1cc(F)cc(S(N)(=O)=NC(=O)Nc2c3c(c(F)c4c2CCC4)CCC3)c1] |
| 205a | 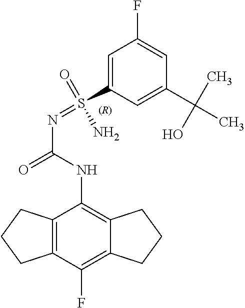 |
| 205b | 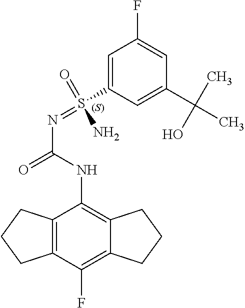 |
| 206 | 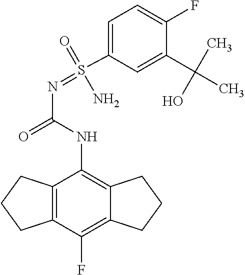 [SMILES: CC(C)(O)c1cc(S(N)(=O)=NC(=O)Nc2c3c(c(F)c4c2CCC4)CCC3)ccc1F] |
| 206a | 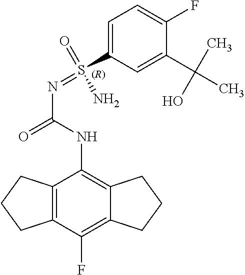 |
| 206b | 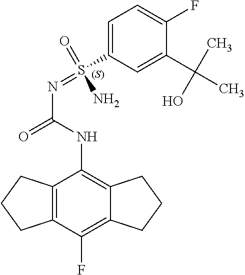 |
| 207a | 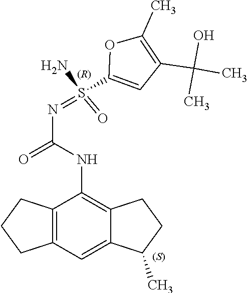 |
| 207b | 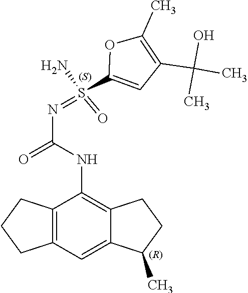 |
| 207bb | 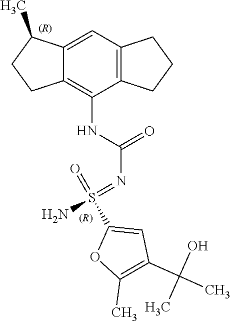 |
| 207aa | 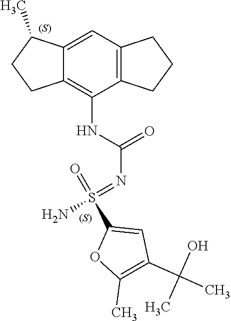 |
| 207c | 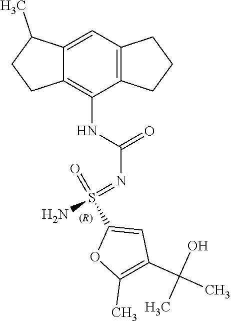 |
| 208 | 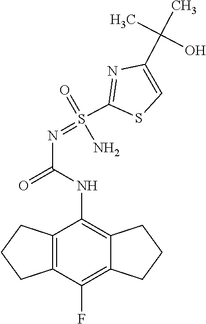 |
| 209 | 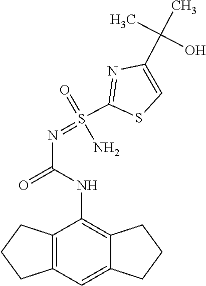 |
| 210 | |
| 211 | 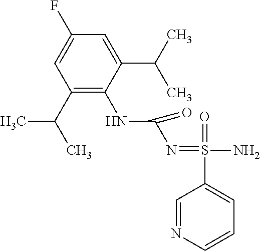 [SMILES: CC(C)c1cc(F)cc(C(C)C)c1NC(=O)N=S(N)(=O)c1cccnc1] |
| 212 | 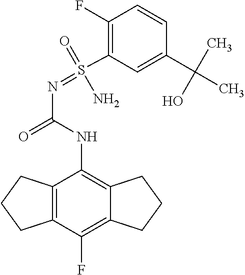 |
| 212a | 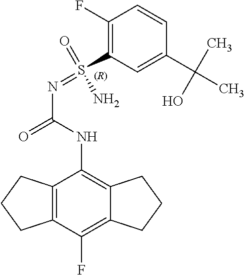 |
| 212b | 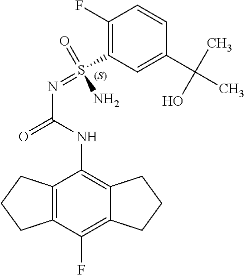 |
| 213 | 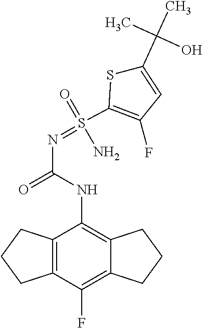 |
| 214 | 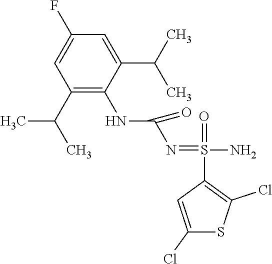 [SMILES: CC(C)c1cc(F)cc(C(C)C)c1NC(=O)N=S(N)(=O)c1cc(Cl)sc1Cl] |
| 215 | 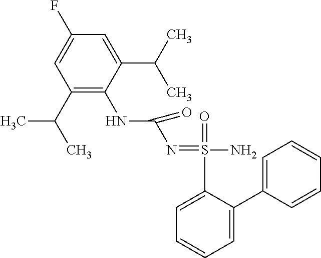 [SMILES: CC(C)c1cc(F)cc(C(C)C)c1NC(=O)N=S(N)(=O)c1ccccc1-c1ccccc1] |
| 216 | |
| 217 | |
| 218 | 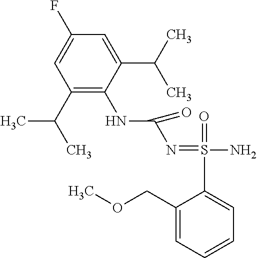 [SMILES: COCc1ccccc1S(N)(=O)=NC(=O)Nc1c(C(C)C)cc(F)cc1C(C)C] |
| 219 | |
| 220 | 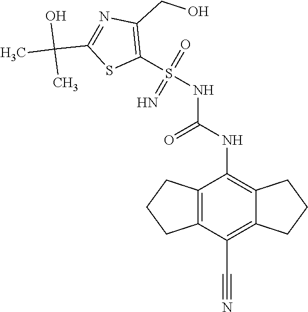 [SMILES: CC(C)(O)c1nc(CO)c(S(=N)(=O)NC(=O)Nc2c3c(c(C#N)c4c2CCC4)CCC3)s1] |
| 220a | 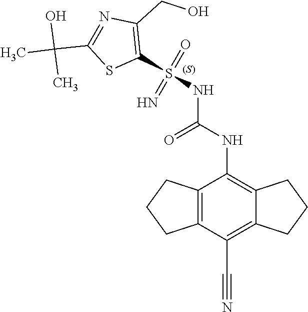 |
| 220b | 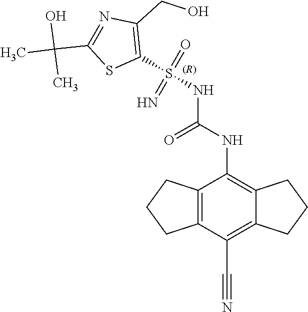 |
| 221 | |
| 223 | |
| 223a | |
| 223b | |
| 225 | |
| 225a | |
| 225b | |
| 226 | 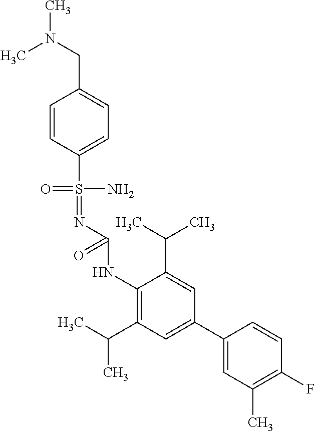 [SMILES: Cc1cc(-c2cc(C(C)C)c(NC(=O)N=S(N)(=O)c3ccc(CN(C)C)cc3)c(C(C)C)c2)ccc1F] |
| 227 | 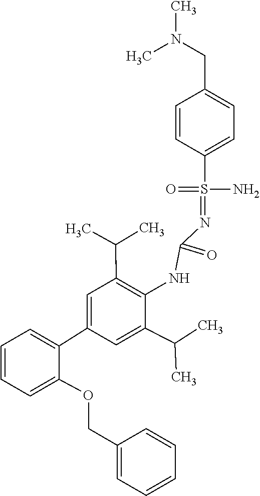 [SMILES: CC(C)c1cc(-c2ccccc2OCc2ccccc2)cc(C(C)C)c1NC(=O)N=S(N)(=O)c1ccc(CN(C)C)cc1] |
| 228 | 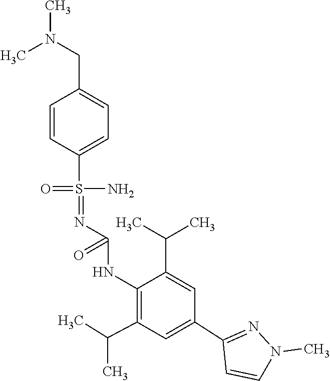 |
| 229 | 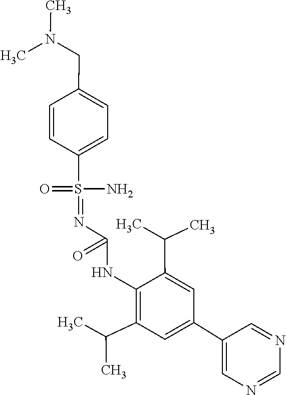 [SMILES: CC(C)c1cc(-c2cncnc2)cc(C(C)C)c1NC(=O)N=S(N)(=O)c1ccc(CN(C)C)cc1] |
| 230 | 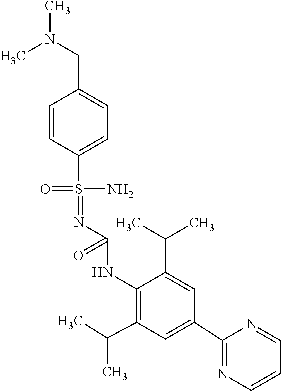 [SMILES: CC(C)c1cc(-c2ncccn2)cc(C(C)C)c1NC(=O)N=S(N)(=O)c1ccc(CN(C)C)cc1] |
| 231 | 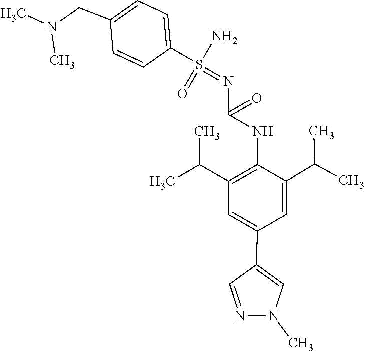 [SMILES: CC(C)c1cc(-c2cnn(C)c2)cc(C(C)C)c1NC(=O)N=S(N)(=O)c1ccc(CN(C)C)cc1] |
| 232 | 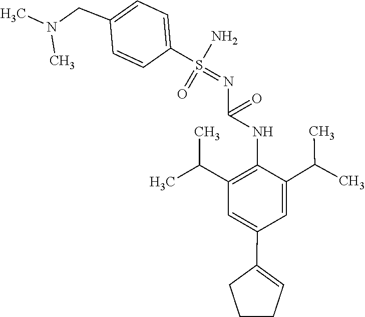 [SMILES: CC(C)c1cc(C2=CCCC2)cc(C(C)C)c1NC(=O)N=S(N)(=O)c1ccc(CN(C)C)cc1] |
| 233 | 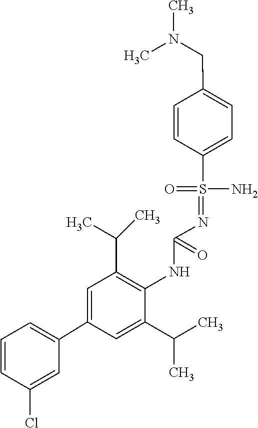 [SMILES: CC(C)c1cc(-c2cccc(Cl)c2)cc(C(C)C)c1NC(=O)N=S(N)(=O)c1ccc(CN(C)C)cc1] |
| 234 | 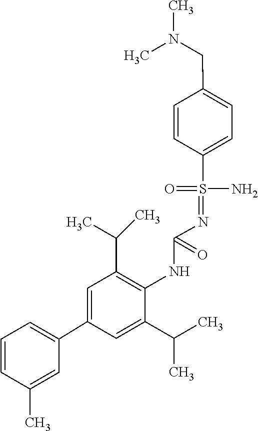 [SMILES: Cc1cccc(-c2cc(C(C)C)c(NC(=O)N=S(N)(=O)c3ccc(CN(C)C)cc3)c(C(C)C)c2)c1] |
| 235 | 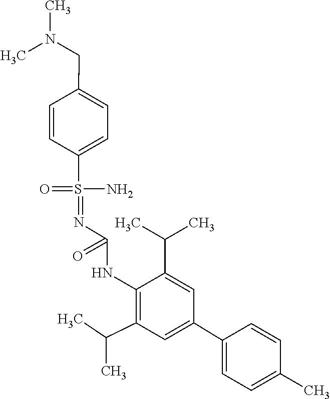 |
| 236 | 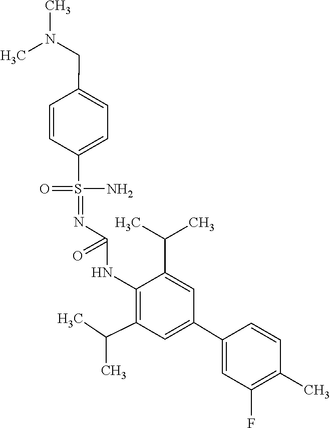 |
| 237 | 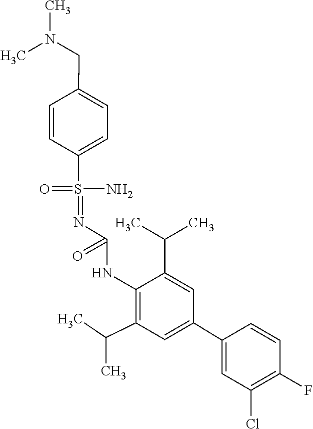 [SMILES: CC(C)c1cc(-c2ccc(F)c(Cl)c2)cc(C(C)C)c1NC(=O)N=S(N)(=O)c1ccc(CN(C)C)cc1] |
| 238 | 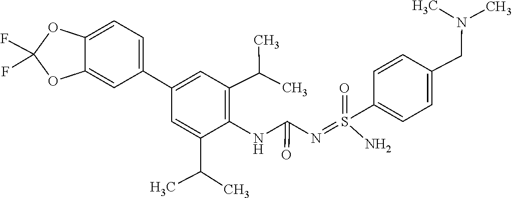 [SMILES: CC(C)c1cc(-c2ccc3c(c2)OC(F)(F)O3)cc(C(C)C)c1NC(=O)N=S(N)(=O)c1ccc(CN(C)C)cc1] |
| 239 | 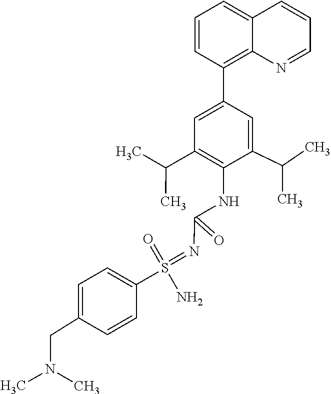 [SMILES: CC(C)c1cc(-c2cccc3cccnc23)cc(C(C)C)c1NC(=O)N=S(N)(=O)c1ccc(CN(C)C)cc1] |
| 240 | 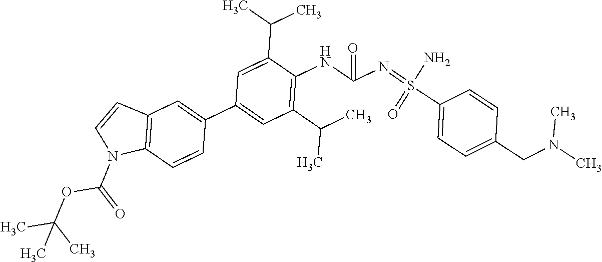 [SMILES: CC(C)c1cc(-c2ccc3c(ccn3C(=O)OC(C)(C)C)c2)cc(C(C)C)c1NC(=O)N=S(N)(=O)c1ccc(CN(C)C)cc1] |
| 241 | 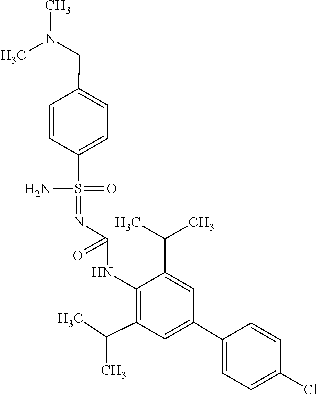 [SMILES: CC(C)c1cc(-c2ccc(Cl)cc2)cc(C(C)C)c1NC(=O)N=S(N)(=O)c1ccc(CN(C)C)cc1] |
| 242 | 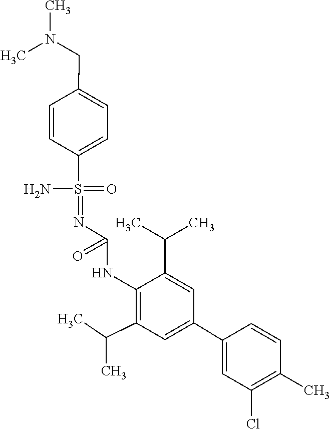 |
| 243 | 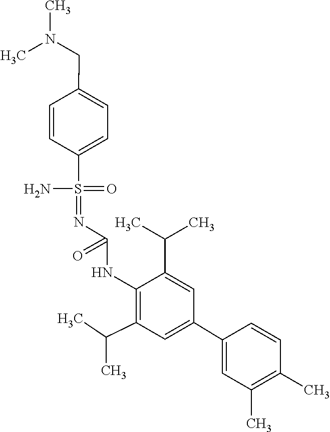 |
| 244 | 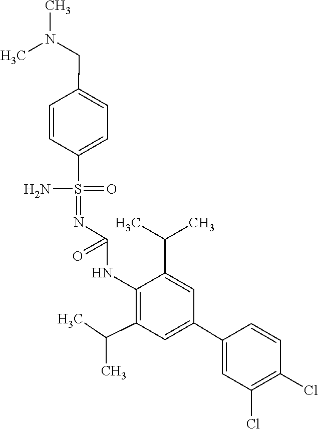 [SMILES: CC(C)c1cc(-c2ccc(Cl)c(Cl)c2)cc(C(C)C)c1NC(=O)N=S(N)(=O)c1ccc(CN(C)C)cc1] |
| 245 | 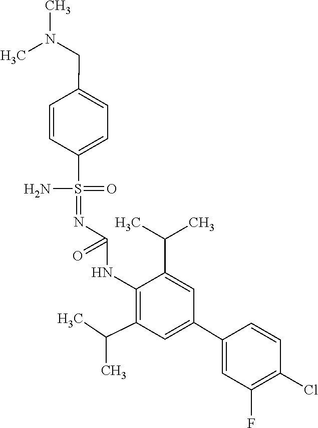 [SMILES: CC(C)c1cc(-c2ccc(Cl)c(F)c2)cc(C(C)C)c1NC(=O)N=S(N)(=O)c1ccc(CN(C)C)cc1] |
| 246 | 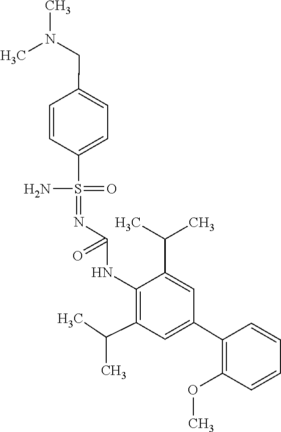 [SMILES: COc1ccccc1-c1cc(C(C)C)c(NC(=O)N=S(N)(=O)c2ccc(CN(C)C)cc2)c(C(C)C)c1] |
| 247 | 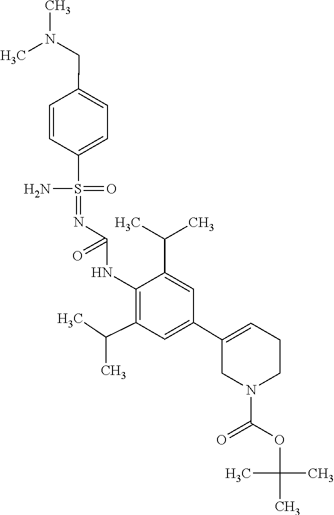 [SMILES: CC(C)c1cc(C2=CCCN(C(=O)OC(C)(C)C)C2)cc(C(C)C)c1NC(=O)N=S(N)(=O)c1ccc(CN(C)C)cc1] |
| 248 | 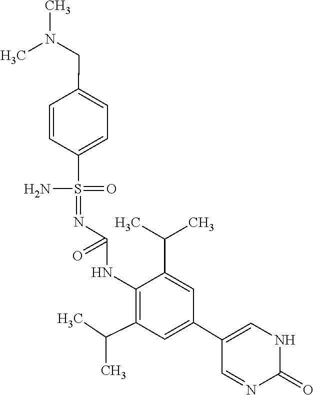 [SMILES: CC(C)c1cc(-c2cnc(=O)[nH]c2)cc(C(C)C)c1NC(=O)N=S(N)(=O)c1ccc(CN(C)C)cc1] |
| 249 | 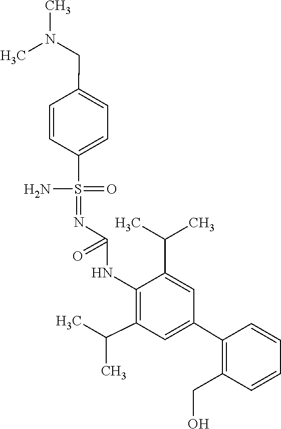 [SMILES: CC(C)c1cc(-c2ccccc2CO)cc(C(C)C)c1NC(=O)N=S(N)(=O)c1ccc(CN(C)C)cc1] |
| 250 | 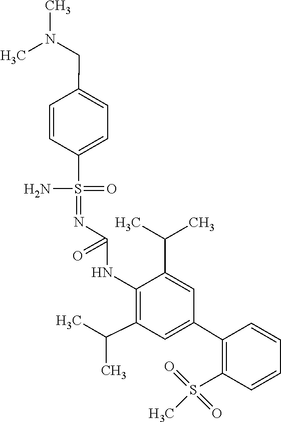 [SMILES: CC(C)c1cc(-c2ccccc2S(C)(=O)=O)cc(C(C)C)c1NC(=O)N=S(N)(=O)c1ccc(CN(C)C)cc1] |
| 251 | 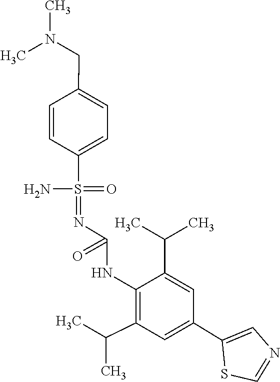 [SMILES: CC(C)c1cc(-c2cncs2)cc(C(C)C)c1NC(=O)N=S(N)(=O)c1ccc(CN(C)C)cc1] |
| 252 | 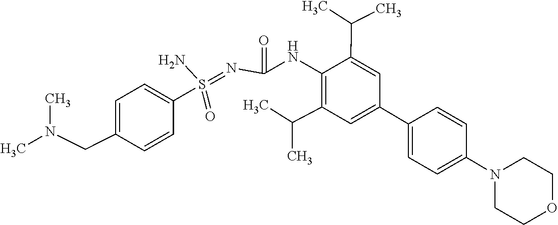 [SMILES: CC(C)c1cc(-c2ccc(N3CCOCC3)cc2)cc(C(C)C)c1NC(=O)N=S(N)(=O)c1ccc(CN(C)C)cc1] |
| 253 | 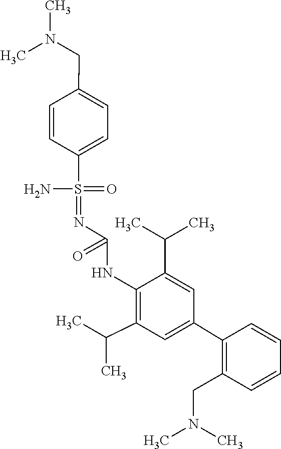 [SMILES: CC(C)c1cc(-c2ccccc2CN(C)C)cc(C(C)C)c1NC(=O)N=S(N)(=O)c1ccc(CN(C)C)cc1] |
| 254 | 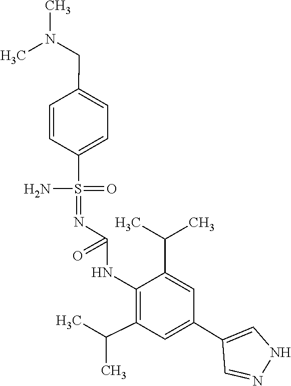 [SMILES: CC(C)c1cc(-c2cn[nH]c2)cc(C(C)C)c1NC(=O)N=S(N)(=O)c1ccc(CN(C)C)cc1] |
| 255 | 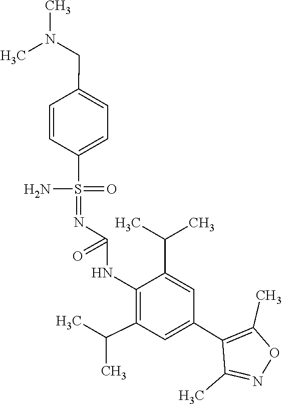 [SMILES: Cc1noc(C)c1-c1cc(C(C)C)c(NC(=O)N=S(N)(=O)c2ccc(CN(C)C)cc2)c(C(C)C)c1] |
| 256 | 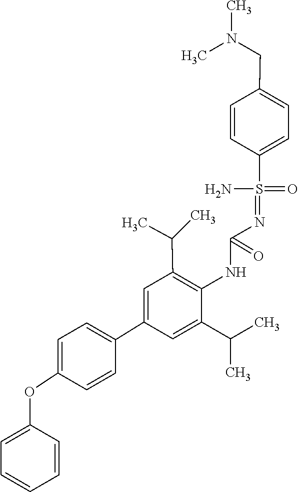 [SMILES: CC(C)c1cc(-c2ccc(Oc3ccccc3)cc2)cc(C(C)C)c1NC(=O)N=S(N)(=O)c1ccc(CN(C)C)cc1] |
| 257 | 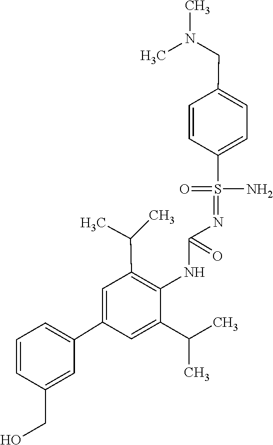 [SMILES: CC(C)c1cc(-c2cccc(CO)c2)cc(C(C)C)c1NC(=O)N=S(N)(=O)c1ccc(CN(C)C)cc1] |
| 258 | 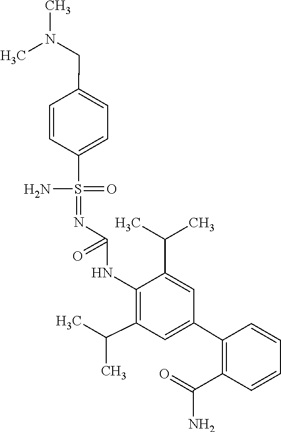 [SMILES: CC(C)c1cc(-c2ccccc2C(N)=O)cc(C(C)C)c1NC(=O)N=S(N)(=O)c1ccc(CN(C)C)cc1] |
| 259 | 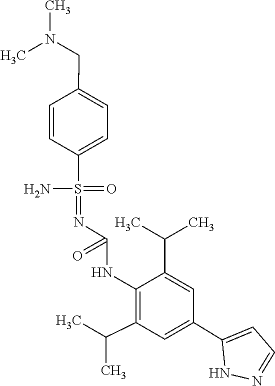 [SMILES: CC(C)c1cc(-c2ccn[nH]2)cc(C(C)C)c1NC(=O)N=S(N)(=O)c1ccc(CN(C)C)cc1] |
| 260 | 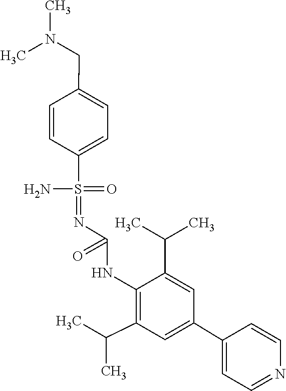 [SMILES: CC(C)c1cc(-c2ccncc2)cc(C(C)C)c1NC(=O)N=S(N)(=O)c1ccc(CN(C)C)cc1] |
| 261 | 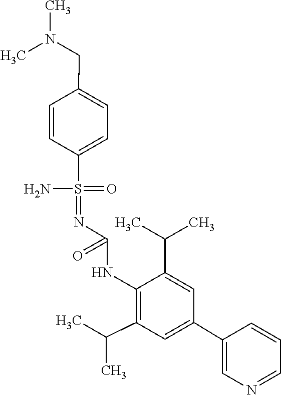 [SMILES: CC(C)c1cc(-c2cccnc2)cc(C(C)C)c1NC(=O)N=S(N)(=O)c1ccc(CN(C)C)cc1] |
| 262 | 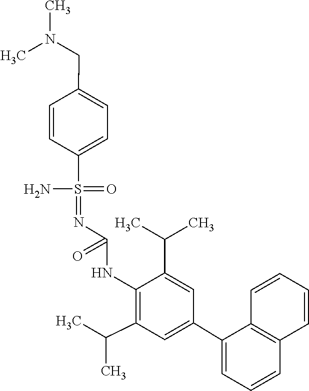 [SMILES: CC(C)c1cc(-c2cccc3ccccc23)cc(C(C)C)c1NC(=O)N=S(N)(=O)c1ccc(CN(C)C)cc1] |
| 263 | 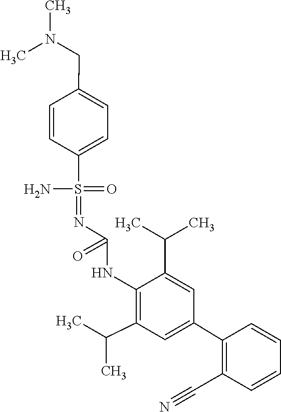 [SMILES: CC(C)c1cc(-c2ccccc2C#N)cc(C(C)C)c1NC(=O)N=S(N)(=O)c1ccc(CN(C)C)cc1] |
| 264 | 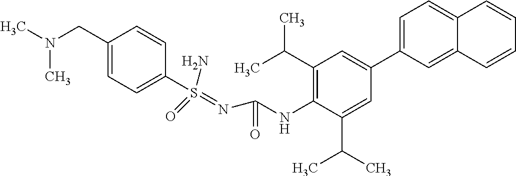 [SMILES: CC(C)c1cc(-c2ccc3ccccc3c2)cc(C(C)C)c1NC(=O)N=S(N)(=O)c1ccc(CN(C)C)cc1] |
| 265 | 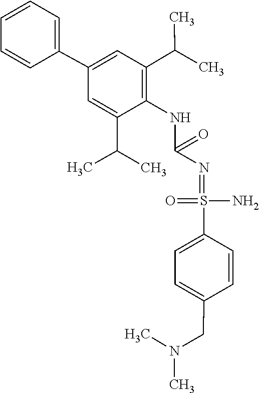 [SMILES: CC(C)c1cc(-c2ccccc2)cc(C(C)C)c1NC(=O)N=S(N)(=O)c1ccc(CN(C)C)cc1] |
| 266 | 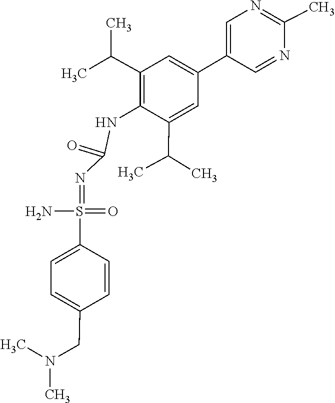 |
| 267 | 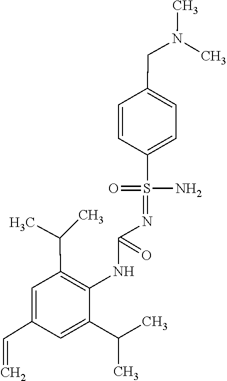 [SMILES: C=Cc1cc(C(C)C)c(NC(=O)N=S(N)(=O)c2ccc(CN(C)C)cc2)c(C(C)C)c1] |
| 268 | 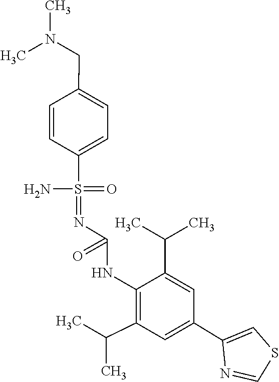 [SMILES: CC(C)c1cc(-c2cscn2)cc(C(C)C)c1NC(=O)N=S(N)(=O)c1ccc(CN(C)C)cc1] |
| 269 | 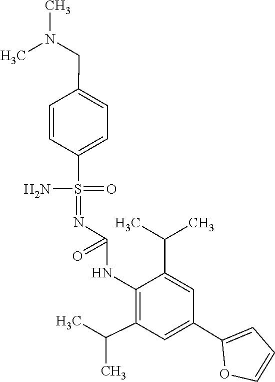 [SMILES: CC(C)c1cc(-c2ccco2)cc(C(C)C)c1NC(=O)N=S(N)(=O)c1ccc(CN(C)C)cc1] |
| 270 | 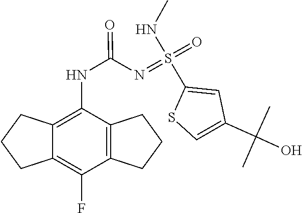 [SMILES: CNS(=O)(=NC(=O)Nc1c2c(c(F)c3c1CCC3)CCC2)c1cc(C(C)(C)O)cs1] |

and pharmaceutically acceptable salts thereof.

In another aspect, an NLRP3 antagonist is a compound selected from the group consisting of the compounds in Table 13 below:

TABLE 13

| | |
|---|---|
| 303 | 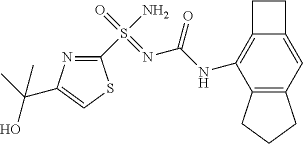 [SMILES: CC(C)(O)c1csc(S(N)(=O)=NC(=O)Nc2c3c(cc4c2CC4)CCC3)n1] |
| 303a | 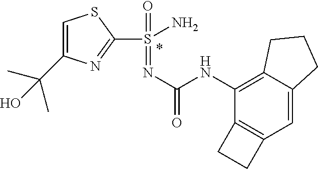 [SMILES: CC(C)(O)c1csc(S(N)(=O)=NC(=O)Nc2c3c(cc4c2CC4)CCC3)n1] |
| 303b | 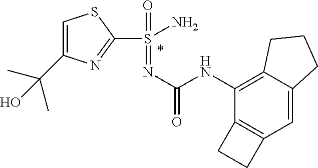 [SMILES: CC(C)(O)c1csc(S(N)(=O)=NC(=O)Nc2c3c(cc4c2CC4)CCC3)n1] |
| 306 | 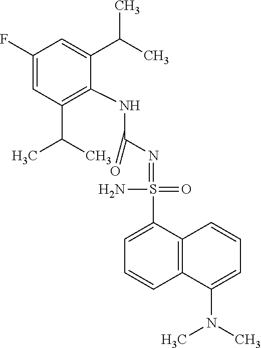 [SMILES: CC(C)c1cc(F)cc(C(C)C)c1NC(=O)N=S(N)(=O)c1cccc2c(N(C)C)cccc12] |
| 307 | 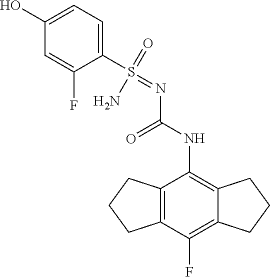 [SMILES: NS(=O)(=NC(=O)Nc1c2c(c(F)c3c1CCC3)CCC2)c1ccc(O)cc1F] |
| 308 | 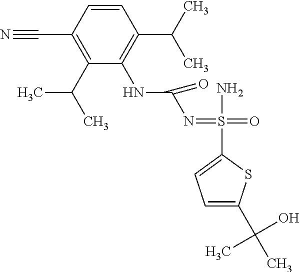 [SMILES: CC(C)c1ccc(C#N)c(C(C)C)c1NC(=O)N=S(N)(=O)c1ccc(C(C)(C)O)s1] |
| 308a | 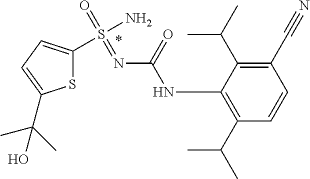 [SMILES: CC(C)c1ccc(C#N)c(C(C)C)c1NC(=O)N=S(N)(=O)c1ccc(C(C)(C)O)s1] |
| 308b | 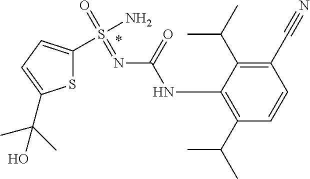 [SMILES: CC(C)c1ccc(C#N)c(C(C)C)c1NC(=O)N=S(N)(=O)c1ccc(C(C)(C)O)s1] |
| 309 | 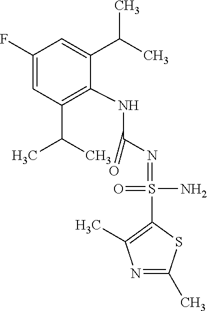 [SMILES: Cc1nc(C)c(S(N)(=O)=NC(=O)Nc2c(C(C)C)cc(F)cc2C(C)C)s1] |
| 310 | 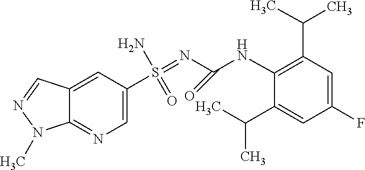 [SMILES: CC(C)c1cc(F)cc(C(C)C)c1NC(=O)N=S(N)(=O)c1cnc2c(cnn2C)c1] |
| 311 | 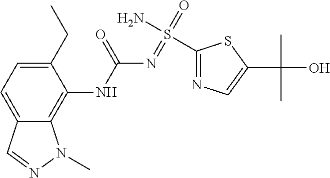 [SMILES: CCc1ccc2cnn(C)c2c1NC(=O)N=S(N)(=O)c1ncc(C(C)(C)O)s1] |
| 312 | |
| 313 | |
| 314 | |
| 315 | 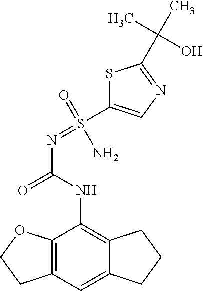 [SMILES: CC(C)(O)c1ncc(S(N)(=O)=NC(=O)Nc2c3c(cc4c2OCC4)CCC3)s1] |
| 315b | |
| 315a | |
| 316 | 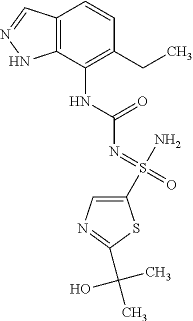 [SMILES: CCc1ccc2cn[nH]c2c1NC(=O)N=S(N)(=O)c1cnc(C(C)(C)O)s1] |
| 316a | 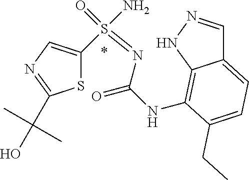 [SMILES: CCc1ccc2cn[nH]c2c1NC(=O)N=S(N)(=O)c1cnc(C(C)(C)O)s1] |
| 316b | 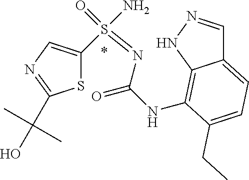 [SMILES: CCc1ccc2cn[nH]c2c1NC(=O)N=S(N)(=O)c1cnc(C(C)(C)O)s1] |
| 317 | 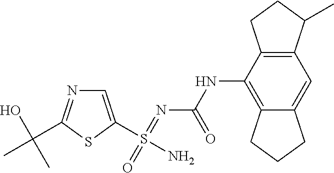 [SMILES: CC1CCc2c1cc1c(c2NC(=O)N=S(N)(=O)c2cnc(C(C)(C)O)s2)CCC1] |
| 317ab | 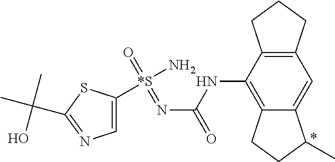 |
| 317aa | 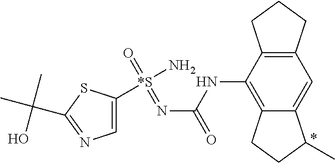 |
| 317bb | 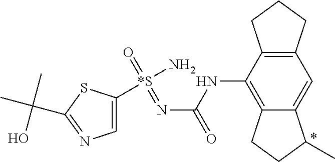 |
| 317ba | 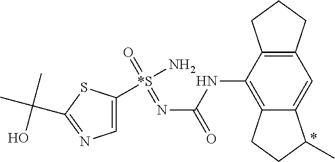 |
| 318 | 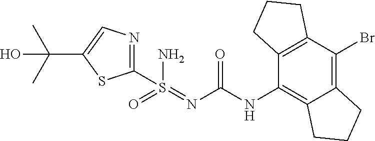 [SMILES: CC(C)(O)c1cnc(S(N)(=O)=NC(=O)Nc2c3c(c(Br)c4c2CCC4)CCC3)s1] |
| 318a | 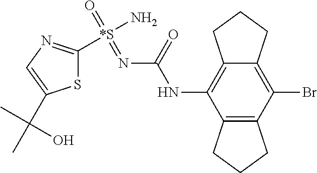 |
| 318b | 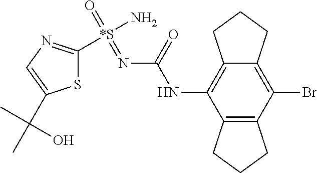 |
| 319 | 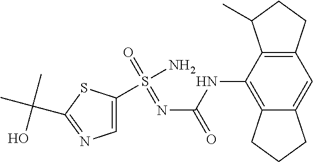 [SMILES: CC1CCc2cc3c(c(NC(=O)N=S(N)(=O)c4cnc(C(C)(C)O)s4)c21)CCC3] |
| 319ab | 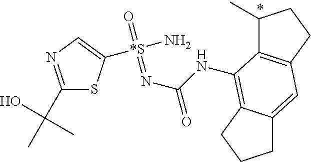 |
| 319ba | 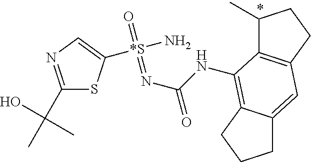 |
| 319aa | 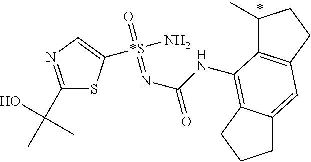 |
| 319bb | 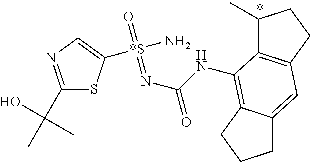 |
| 320 | 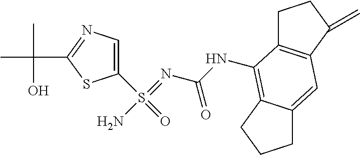 [SMILES: C=C1CCc2c1cc1c(c2NC(=O)N=S(N)(=O)c2cnc(C(C)(C)O)s2)CCC1] |
| 320a | 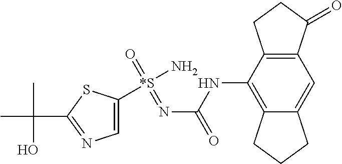 |
| 320b | 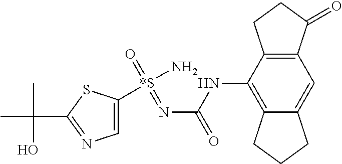 |
| 321 | 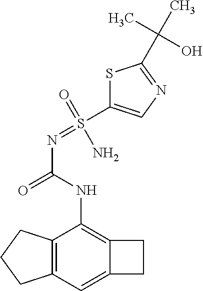 [SMILES: CC(C)(O)c1ncc(S(N)(=O)=NC(=O)Nc2c3c(cc4c2CC4)CCC3)s1] |
| 321b | 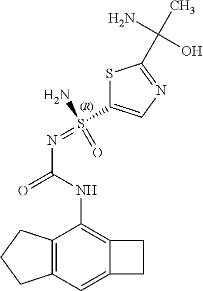 |
| 321a | 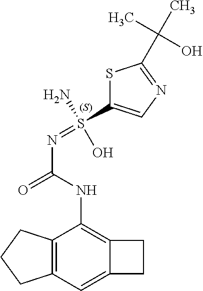 |
| 322 | 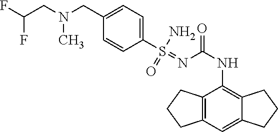 [SMILES: CN(Cc1ccc(S(N)(=O)=NC(=O)Nc2c3c(cc4c2CCC4)CCC3)cc1)CC(F)F] |
| 323 | 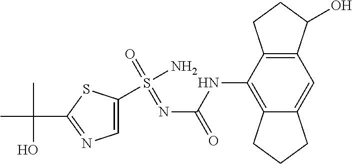 [SMILES: CC(C)(O)c1ncc(S(N)(=O)=NC(=O)Nc2c3c(cc4c2CCC4O)CCC3)s1] |
| 323ab | 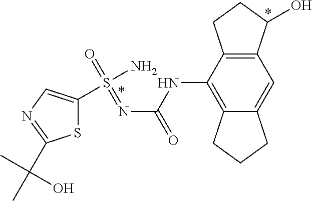 [SMILES: CC(C)(O)c1ncc(S(N)(=O)=NC(=O)Nc2c3c(cc4c2CCC4O)CCC3)s1] |
| 323aa | 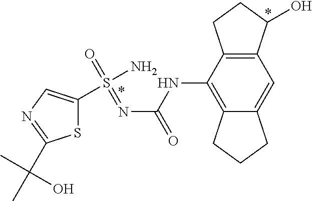 [SMILES: CC(C)(O)c1ncc(S(N)(=O)=NC(=O)Nc2c3c(cc4c2CCC4O)CCC3)s1] |
| 323bb | 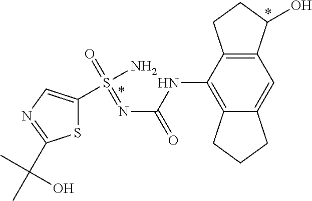 [SMILES: CC(C)(O)c1ncc(S(N)(=O)=NC(=O)Nc2c3c(cc4c2CCC4O)CCC3)s1] |
| 323ba | 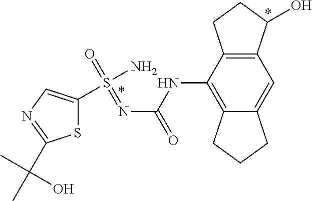 [SMILES: CC(C)(O)c1ncc(S(N)(=O)=NC(=O)Nc2c3c(cc4c2CCC4O)CCC3)s1] |
| 324 | 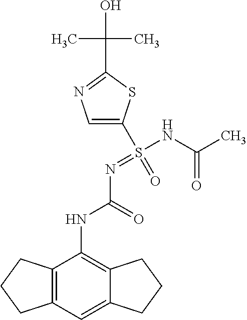 [SMILES: CC(=O)NS(=O)(=NC(=O)Nc1c2c(cc3c1CCC3)CCC2)c1cnc(C(C)(C)O)s1] |
| 325 | 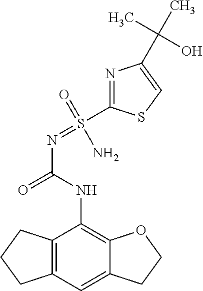 [SMILES: CC(C)(O)c1csc(S(N)(=O)=NC(=O)Nc2c3c(cc4c2OCC4)CCC3)n1] |
| 325a | 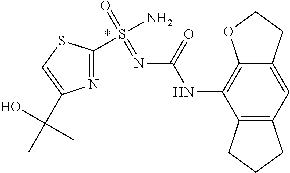 [SMILES: CC(C)(O)c1csc(S(N)(=O)=NC(=O)Nc2c3c(cc4c2OCC4)CCC3)n1] |
| 325b | 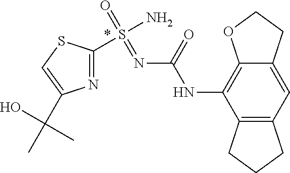 [SMILES: CC(C)(O)c1csc(S(N)(=O)=NC(=O)Nc2c3c(cc4c2OCC4)CCC3)n1] |
| 326 | 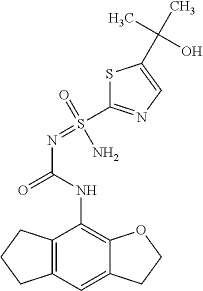 [SMILES: CC(C)(O)c1cnc(S(N)(=O)=NC(=O)Nc2c3c(cc4c2OCC4)CCC3)s1] |
| 326b | 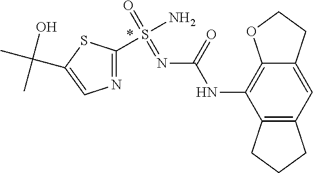 [SMILES: CC(C)(O)c1cnc(S(N)(=O)=NC(=O)Nc2c3c(cc4c2OCC4)CCC3)s1] |
| 326a | 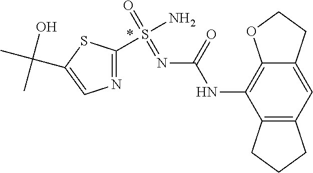 [SMILES: CC(C)(O)c1cnc(S(N)(=O)=NC(=O)Nc2c3c(cc4c2OCC4)CCC3)s1] |
| 327 | 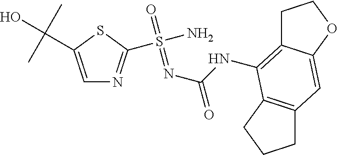 [SMILES: CC(C)(O)c1cnc(S(N)(=O)=NC(=O)Nc2c3c(cc4c2CCO4)CCC3)s1] |
| 328b | 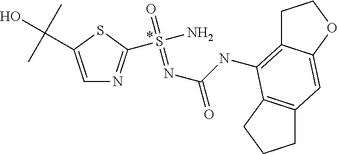 [SMILES: CC(C)(O)c1cnc(S(N)(=O)=NC(=O)Nc2c3c(cc4c2CCO4)CCC3)s1] |
| 328a | 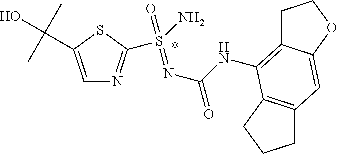 [SMILES: CC(C)(O)c1cnc(S(N)(=O)=NC(=O)Nc2c3c(cc4c2CCO4)CCC3)s1] |
| 329 | 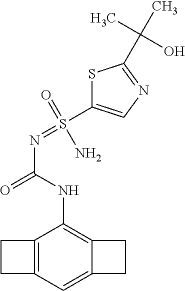 [SMILES: CC(C)(O)c1ncc(S(N)(=O)=NC(=O)Nc2c3c(cc4c2CC4)CC3)s1] |
| 329a | 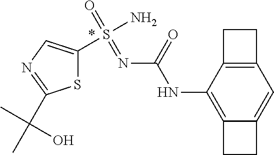 [SMILES: CC(C)(O)c1ncc(S(N)(=O)=NC(=O)Nc2c3c(cc4c2CC4)CC3)s1] |
| 329b | 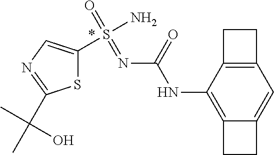 [SMILES: CC(C)(O)c1ncc(S(N)(=O)=NC(=O)Nc2c3c(cc4c2CC4)CC3)s1] |
| 330 | 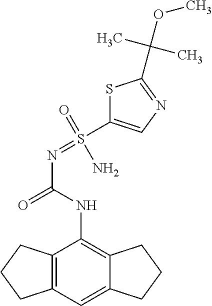 [SMILES: COC(C)(C)c1ncc(S(N)(=O)=NC(=O)Nc2c3c(cc4c2CCC4)CCC3)s1] |
| 330a | 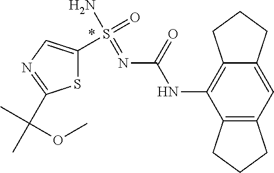 [SMILES: COC(C)(C)c1ncc(S(N)(=O)=NC(=O)Nc2c3c(cc4c2CCC4)CCC3)s1] |
| 330b | 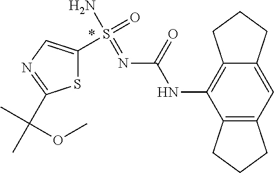 [SMILES: COC(C)(C)c1ncc(S(N)(=O)=NC(=O)Nc2c3c(cc4c2CCC4)CCC3)s1] |
| 331 | 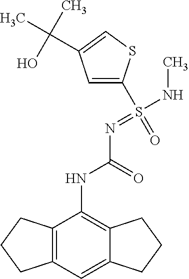 [SMILES: CNS(=O)(=NC(=O)Nc1c2c(cc3c1CCC3)CCC2)c1cc(C(C)(C)O)cs1] |
| 332 | 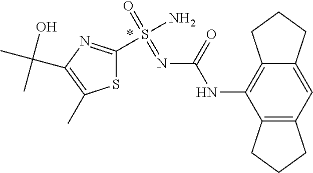 [SMILES: Cc1sc(S(N)(=O)=NC(=O)Nc2c3c(cc4c2CCC4)CCC3)nc1C(C)(C)O] |
| 332a | 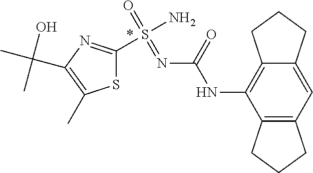 [SMILES: Cc1sc(S(N)(=O)=NC(=O)Nc2c3c(cc4c2CCC4)CCC3)nc1C(C)(C)O] |
| 332b | 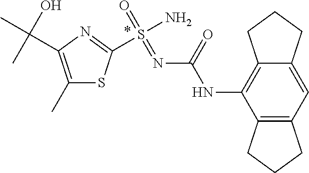 [SMILES: Cc1sc(S(N)(=O)=NC(=O)Nc2c3c(cc4c2CCC4)CCC3)nc1C(C)(C)O] |
| 333 | 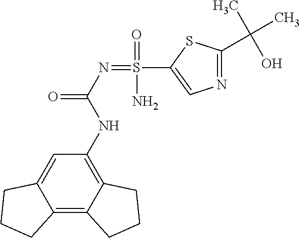 [SMILES: CC(C)(O)c1ncc(S(N)(=O)=NC(=O)Nc2cc3c(c4c2CCC4)CCC3)s1] |
| 333a | 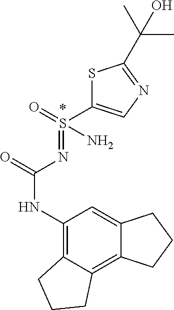 [SMILES: CC(C)(O)c1ncc(S(N)(=O)=NC(=O)Nc2cc3c(c4c2CCC4)CCC3)s1] |
| 333b | 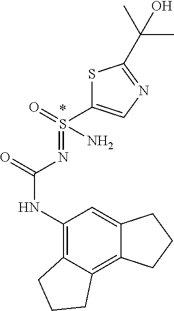 [SMILES: CC(C)(O)c1ncc(S(N)(=O)=NC(=O)Nc2cc3c(c4c2CCC4)CCC3)s1] |
| 334 | 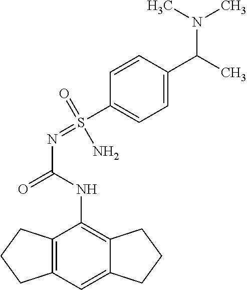 [SMILES: CC(c1ccc(S(N)(=O)=NC(=O)Nc2c3c(cc4c2CCC4)CCC3)cc1)N(C)C] |
| 334ba | 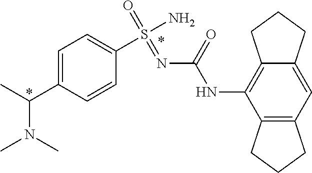 [SMILES: CC(c1ccc(S(N)(=O)=NC(=O)Nc2c3c(cc4c2CCC4)CCC3)cc1)N(C)C] |
| 334bb | 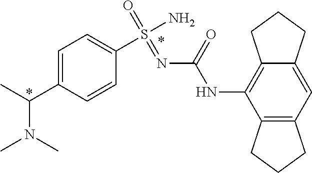 [SMILES: CC(c1ccc(S(N)(=O)=NC(=O)Nc2c3c(cc4c2CCC4)CCC3)cc1)N(C)C] |
| 334aa | 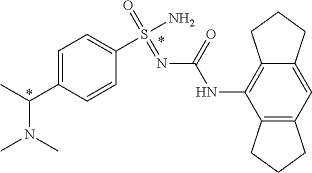 [SMILES: CC(c1ccc(S(N)(=O)=NC(=O)Nc2c3c(cc4c2CCC4)CCC3)cc1)N(C)C] |
| 334ab | 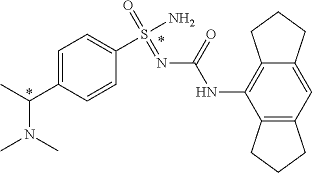 [SMILES: CC(c1ccc(S(N)(=O)=NC(=O)Nc2c3c(cc4c2CCC4)CCC3)cc1)N(C)C] |
| 334b | 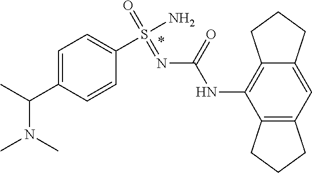 [SMILES: CC(c1ccc(S(N)(=O)=NC(=O)Nc2c3c(cc4c2CCC4)CCC3)cc1)N(C)C] |
| 334a | 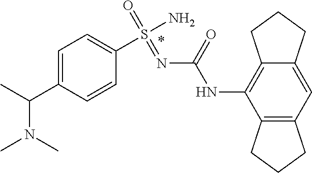 [SMILES: CC(c1ccc(S(N)(=O)=NC(=O)Nc2c3c(cc4c2CCC4)CCC3)cc1)N(C)C] |
| 335 | 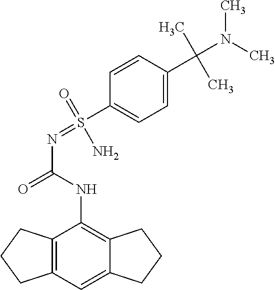 [SMILES: CN(C)C(C)(C)c1ccc(S(N)(=O)=NC(=O)Nc2c3c(cc4c2CCC4)CCC3)cc1] |
| 335b | 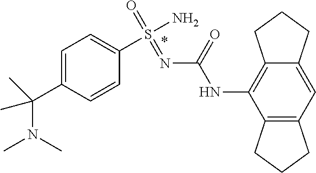 [SMILES: CN(C)C(C)(C)c1ccc(S(N)(=O)=NC(=O)Nc2c3c(cc4c2CCC4)CCC3)cc1] |
| 335a | 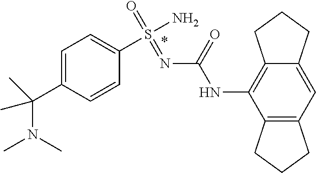 [SMILES: CN(C)C(C)(C)c1ccc(S(N)(=O)=NC(=O)Nc2c3c(cc4c2CCC4)CCC3)cc1] |
| 336 | 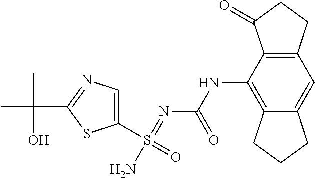 [SMILES: CC(C)(O)c1ncc(S(N)(=O)=NC(=O)Nc2c3c(cc4c2C(=O)CC4)CCC3)s1] |
| 336a | 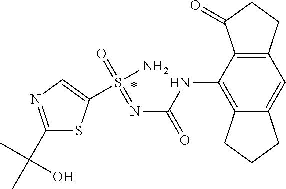 [SMILES: CC(C)(O)c1ncc(S(N)(=O)=NC(=O)Nc2c3c(cc4c2C(=O)CC4)CCC3)s1] |
| 336b | 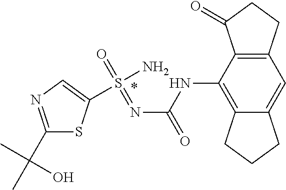 [SMILES: CC(C)(O)c1ncc(S(N)(=O)=NC(=O)Nc2c3c(cc4c2C(=O)CC4)CCC3)s1] |
| 337 | 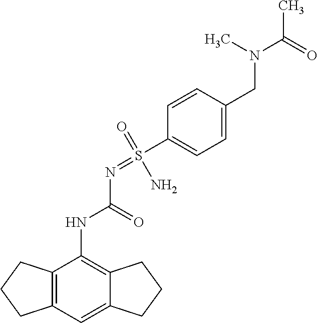 [SMILES: CC(=O)N(C)Cc1ccc(S(N)(=O)=NC(=O)Nc2c3c(cc4c2CCC4)CCC3)cc1] |
| 337a | 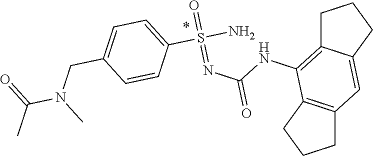 [SMILES: CC(=O)N(C)Cc1ccc(S(N)(=O)=NC(=O)Nc2c3c(cc4c2CCC4)CCC3)cc1] |
| 337b | 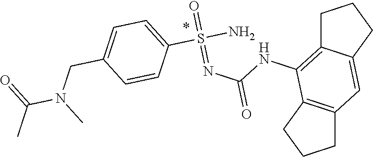 [SMILES: CC(=O)N(C)Cc1ccc(S(N)(=O)=NC(=O)Nc2c3c(cc4c2CCC4)CCC3)cc1] |
| 338 | 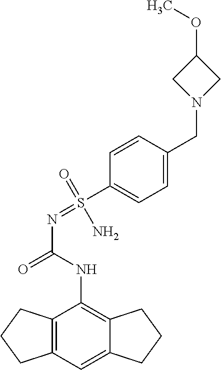 [SMILES: COC1CN(Cc2ccc(S(N)(=O)=NC(=O)Nc3c4c(cc5c3CCC5)CCC4)cc2)C1] |
| 338a | 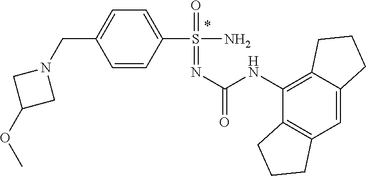 [SMILES: COC1CN(Cc2ccc(S(N)(=O)=NC(=O)Nc3c4c(cc5c3CCC5)CCC4)cc2)C1] |
| 338b | 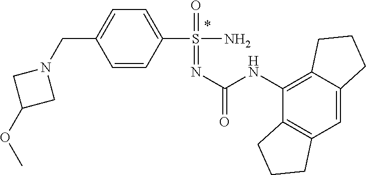 [SMILES: COC1CN(Cc2ccc(S(N)(=O)=NC(=O)Nc3c4c(cc5c3CCC5)CCC4)cc2)C1] |
| 339 | 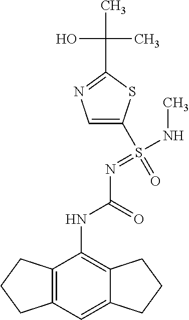 [SMILES: CNS(=O)(=NC(=O)Nc1c2c(cc3c1CCC3)CCC2)c1cnc(C(C)(C)O)s1] |
| 339a | 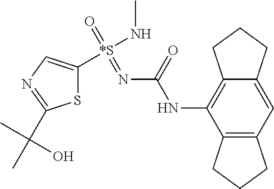 |
| 339b | 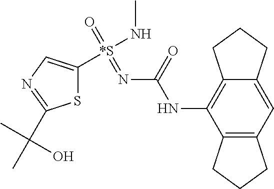 |
| 340 | 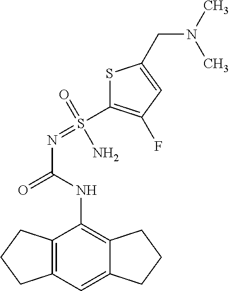 [SMILES: CN(C)Cc1cc(F)c(S(N)(=O)=NC(=O)Nc2c3c(cc4c2CCC4)CCC3)s1] |
| 340a | 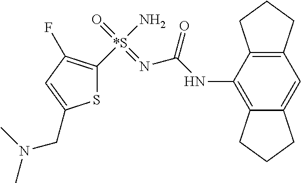 |
| 340b | 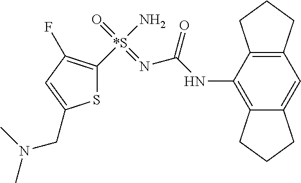 |
| 341 | 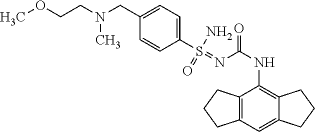 [SMILES: COCCN(C)Cc1ccc(S(N)(=O)=NC(=O)Nc2c3c(cc4c2CCC4)CCC3)cc1] |
| 341b | 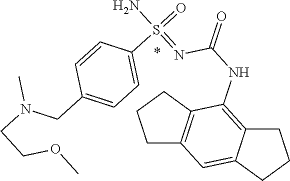 [SMILES: COCCN(C)Cc1ccc(S(N)(=O)=NC(=O)Nc2c3c(cc4c2CCC4)CCC3)cc1] |
| 341a | 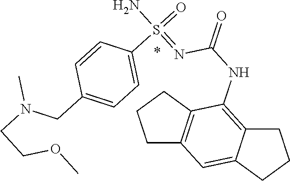 [SMILES: COCCN(C)Cc1ccc(S(N)(=O)=NC(=O)Nc2c3c(cc4c2CCC4)CCC3)cc1] |
| 342 | 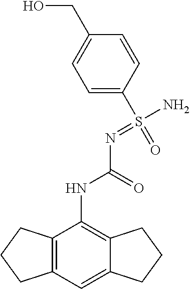 [SMILES: NS(=O)(=NC(=O)Nc1c2c(cc3c1CCC3)CCC2)c1ccc(CO)cc1] |
| 343 | 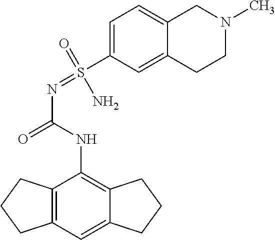 [SMILES: CN1CCc2cc(S(N)(=O)=NC(=O)Nc3c4c(cc5c3CCC5)CCC4)ccc2C1] |
| 343a | 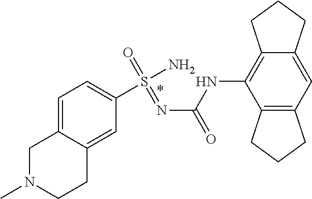 [SMILES: CN1CCc2cc(S(N)(=O)=NC(=O)Nc3c4c(cc5c3CCC5)CCC4)ccc2C1] |
| 343b | 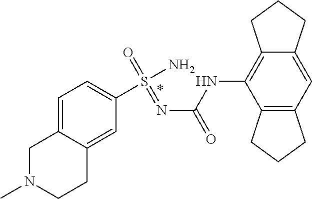 [SMILES: CN1CCc2cc(S(N)(=O)=NC(=O)Nc3c4c(cc5c3CCC5)CCC4)ccc2C1] |
| 344 | 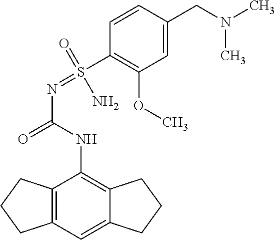 [SMILES: COc1cc(CN(C)C)ccc1S(N)(=O)=NC(=O)Nc1c2c(cc3c1CCC3)CCC2] |
| 345 | 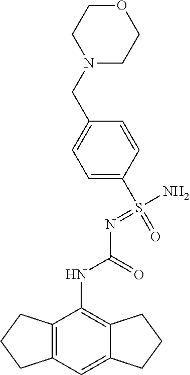 [SMILES: NS(=O)(=NC(=O)Nc1c2c(cc3c1CCC3)CCC2)c1ccc(CN2CCOCC2)cc1] |
| 346 | 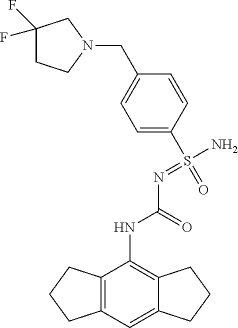 [SMILES: NS(=O)(=NC(=O)Nc1c2c(cc3c1CCC3)CCC2)c1ccc(CN2CCC(F)(F)C2)cc1] |
| 347 | 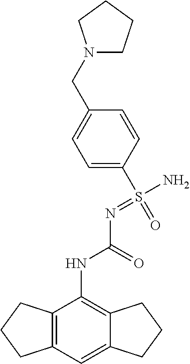 [SMILES: NS(=O)(=NC(=O)Nc1c2c(cc3c1CCC3)CCC2)c1ccc(CN2CCCC2)cc1] |
| 348 | 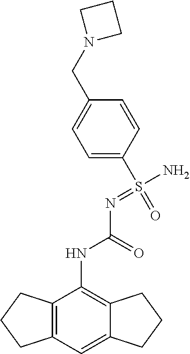 [SMILES: NS(=O)(=NC(=O)Nc1c2c(cc3c1CCC3)CCC2)c1ccc(CN2CCC2)cc1] |
| 349 | 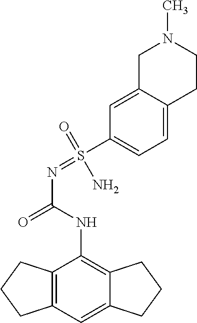 [SMILES: CN1CCc2ccc(S(N)(=O)=NC(=O)Nc3c4c(cc5c3CCC5)CCC4)cc2C1] |
| 350 | 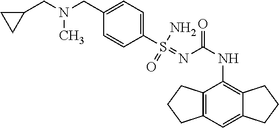 [SMILES: CN(Cc1ccc(S(N)(=O)=NC(=O)Nc2c3c(cc4c2CCC4)CCC3)cc1)CC1CC1] |
| 351 | 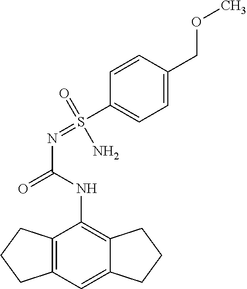 [SMILES: COCc1ccc(S(N)(=O)=NC(=O)Nc2c3c(cc4c2CCC4)CCC3)cc1] |
| 352 | 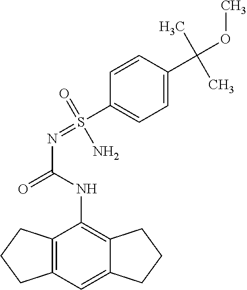 [SMILES: COC(C)(C)c1ccc(S(N)(=O)=NC(=O)Nc2c3c(cc4c2CCC4)CCC3)cc1] |
| 352b | 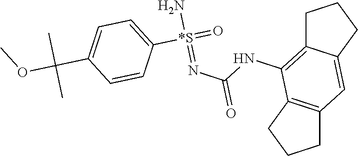 |
| 352a | 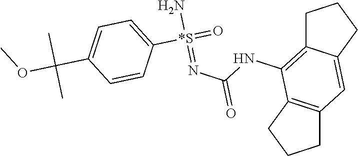 |
| 353 | 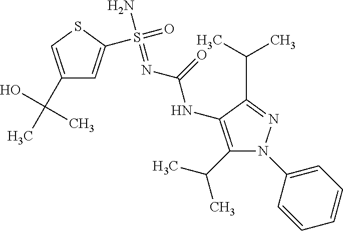 [SMILES: CC(C)c1nn(-c2ccccc2)c(C(C)C)c1NC(=O)N=S(N)(=O)c1cc(C(C)(C)O)cs1] |
| 354 | 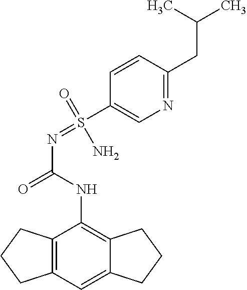 [SMILES: CC(C)Cc1ccc(S(N)(=O)=NC(=O)Nc2c3c(cc4c2CCC4)CCC3)cn1] |
| 354a | 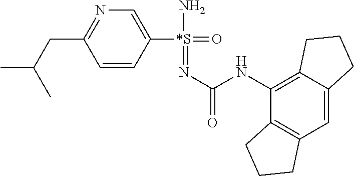 |
| 354b | 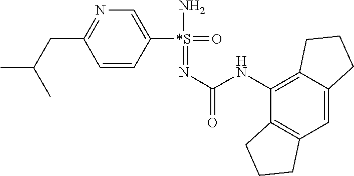 |
| 355 | 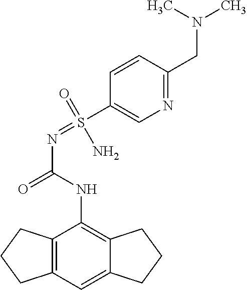 [SMILES: CN(C)Cc1ccc(S(N)(=O)=NC(=O)Nc2c3c(cc4c2CCC4)CCC3)cn1] |
| 356 | 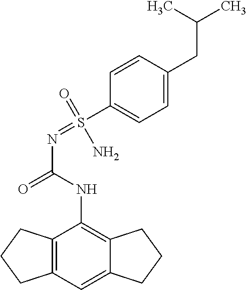 [SMILES: CC(C)Cc1ccc(S(N)(=O)=NC(=O)Nc2c3c(cc4c2CCC4)CCC3)cc1] |
| 357 | 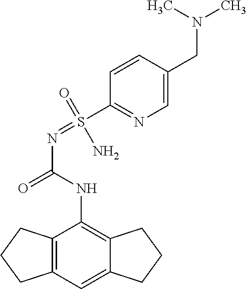 [SMILES: CN(C)Cc1ccc(S(N)(=O)=NC(=O)Nc2c3c(cc4c2CCC4)CCC3)nc1] |
| 357a | 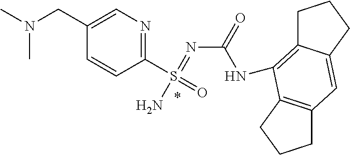 [SMILES: CN(C)Cc1ccc(S(N)(=O)=NC(=O)Nc2c3c(cc4c2CCC4)CCC3)nc1] |
| 357b | 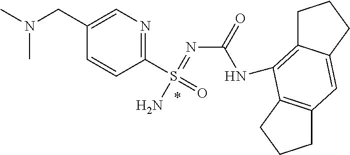 [SMILES: CN(C)Cc1ccc(S(N)(=O)=NC(=O)Nc2c3c(cc4c2CCC4)CCC3)nc1] |
| 358 | 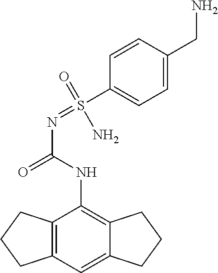 [SMILES: NCc1ccc(S(N)(=O)=NC(=O)Nc2c3c(cc4c2CCC4)CCC3)cc1] |
| 359 | 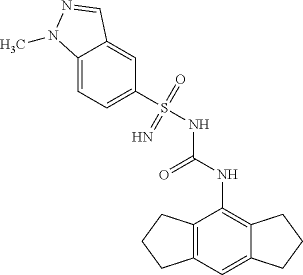 [SMILES: Cn1ncc2cc(S(=N)(=O)NC(=O)Nc3c4c(cc5c3CCC5)CCC4)ccc21] |
| 359a | 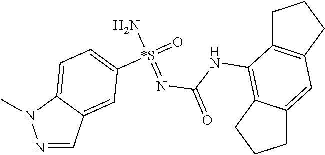 |
| 359b | 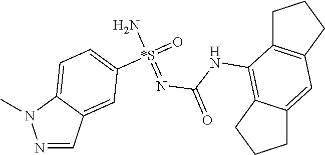 |
| 360ba | 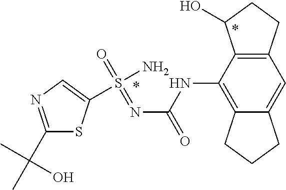 [SMILES: CC(C)(O)c1ncc(S(N)(=O)=NC(=O)Nc2c3c(cc4c2C(O)CC4)CCC3)s1] |
| 360bb | 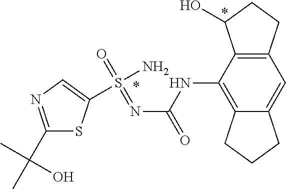 [SMILES: CC(C)(O)c1ncc(S(N)(=O)=NC(=O)Nc2c3c(cc4c2C(O)CC4)CCC3)s1] |
| 361b | 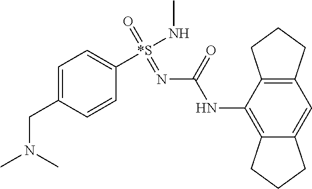 |
| 361a | 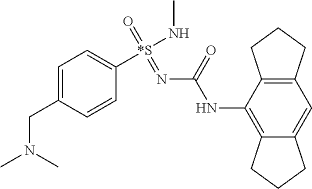 |
| 363b | 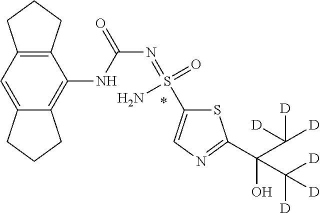 [SMILES: [2H]C([2H])([2H])C(O)(c1ncc(S(N)(=O)=NC(=O)Nc2c3c(cc4c2CCC4)CCC3)s1)C([2H])([2H])[2H]] |
| 363a | 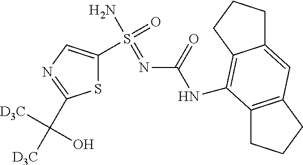 [SMILES: [2H]C([2H])([2H])C(O)(c1ncc(S(N)(=O)=NC(=O)Nc2c3c(cc4c2CCC4)CCC3)s1)C([2H])([2H])[2H]] |
| 364a | 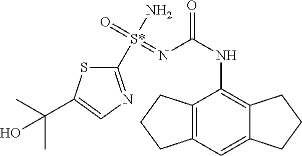 |
| 364b | 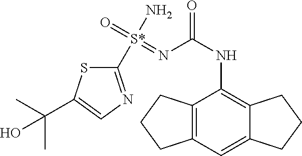 |
| 365a | 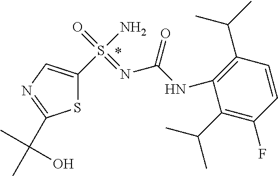 |
| 365b | 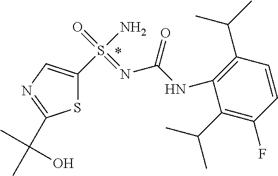 |
| 366a | 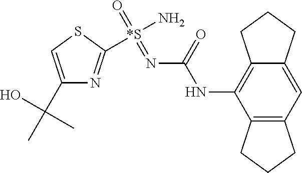 |
| 366b | 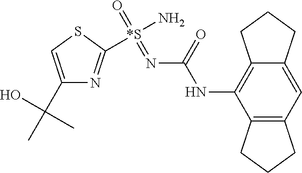 |
| 367a | 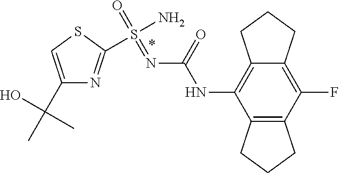 [SMILES: CC(C)(O)c1csc(S(N)(=O)=NC(=O)Nc2c3c(c(F)c4c2CCC4)CCC3)n1] |
| 367b | 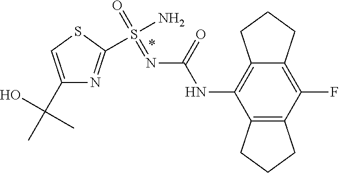 [SMILES: CC(C)(O)c1csc(S(N)(=O)=NC(=O)Nc2c3c(c(F)c4c2CCC4)CCC3)n1] |
| 369a | 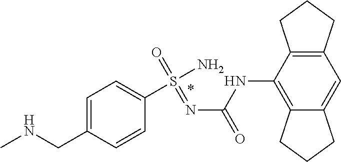 [SMILES: CNCc1ccc(S(N)(=O)=NC(=O)Nc2c3c(cc4c2CCC4)CCC3)cc1] |
| 369b | 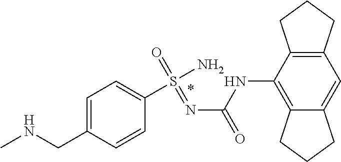 [SMILES: CNCc1ccc(S(N)(=O)=NC(=O)Nc2c3c(cc4c2CCC4)CCC3)cc1] |
| 371a | 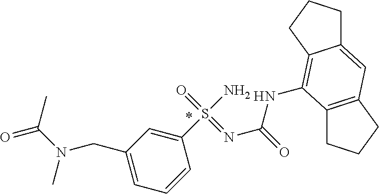 [SMILES: CC(=O)N(C)Cc1cccc(S(N)(=O)=NC(=O)Nc2c3c(cc4c2CCC4)CCC3)c1] |
| 371b | 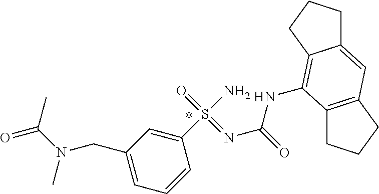 [SMILES: CC(=O)N(C)Cc1cccc(S(N)(=O)=NC(=O)Nc2c3c(cc4c2CCC4)CCC3)c1] |
| 372a | 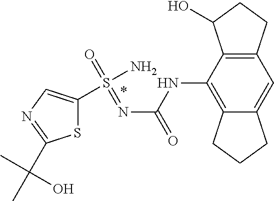 [SMILES: CC(C)(O)c1ncc(S(N)(=O)=NC(=O)Nc2c3c(cc4c2C(O)CC4)CCC3)s1] |
| 372b | 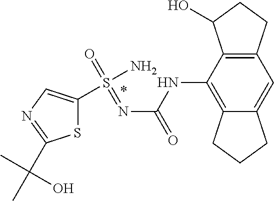 [SMILES: CC(C)(O)c1ncc(S(N)(=O)=NC(=O)Nc2c3c(cc4c2C(O)CC4)CCC3)s1] |
| 373a | 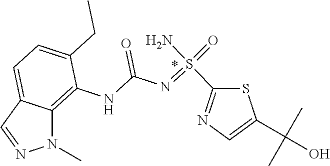 [SMILES: CCc1ccc2cnn(C)c2c1NC(=O)N=S(N)(=O)c1ncc(C(C)(C)O)s1] |
| 373b | 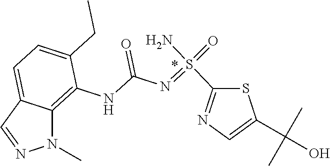 [SMILES: CCc1ccc2cnn(C)c2c1NC(=O)N=S(N)(=O)c1ncc(C(C)(C)O)s1] |
| 374a | 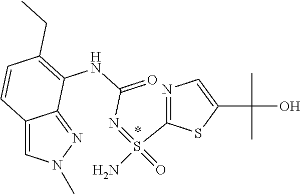 [SMILES: CCc1ccc2cn(C)nc2c1NC(=O)N=S(N)(=O)c1ncc(C(C)(C)O)s1] |
| 374b | 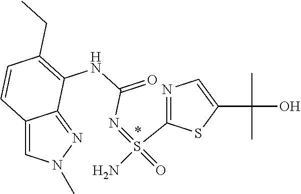 [SMILES: CCc1ccc2cn(C)nc2c1NC(=O)N=S(N)(=O)c1ncc(C(C)(C)O)s1] |
| 375 | 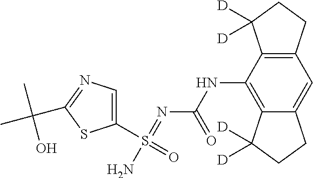 [SMILES: [2H]C1([2H])CCc2cc3c(c(NC(=O)N=S(N)(=O)c4cnc(C(C)(C)O)s4)c21)C([2H])([2H])CC3] |
| 375a | 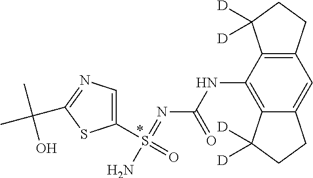 [SMILES: [2H]C1([2H])CCc2cc3c(c(NC(=O)N=S(N)(=O)c4cnc(C(C)(C)O)s4)c21)C([2H])([2H])CC3] |
| 375b | 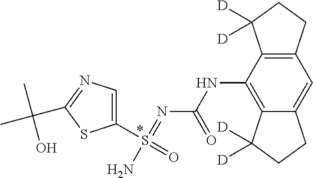 [SMILES: [2H]C1([2H])CCc2cc3c(c(NC(=O)N=S(N)(=O)c4cnc(C(C)(C)O)s4)c21)C([2H])([2H])CC3] |
| 376 | 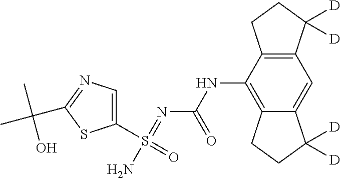 [SMILES: [2H]C1([2H])CCc2c1cc1c(c2NC(=O)N=S(N)(=O)c2cnc(C(C)(C)O)s2)CCC1([2H])[2H]] |
| 376a | 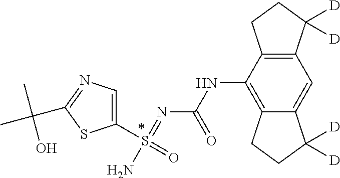 [SMILES: [2H]C1([2H])CCc2c1cc1c(c2NC(=O)N=S(N)(=O)c2cnc(C(C)(C)O)s2)CCC1([2H])[2H]] |
| 376b | 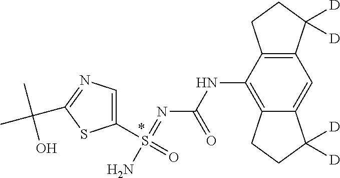 [SMILES: [2H]C1([2H])CCc2c1cc1c(c2NC(=O)N=S(N)(=O)c2cnc(C(C)(C)O)s2)CCC1([2H])[2H]] |
| 377 | 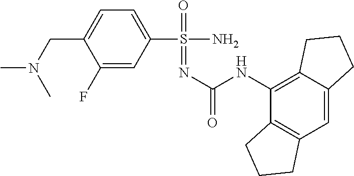 [SMILES: CN(C)Cc1ccc(S(N)(=O)=NC(=O)Nc2c3c(cc4c2CCC4)CCC3)cc1F] |
| 378 | 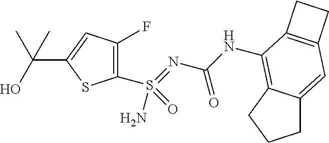 [SMILES: CC(C)(O)c1cc(F)c(S(N)(=O)=NC(=O)Nc2c3c(cc4c2CC4)CCC3)s1] |
| 379 | 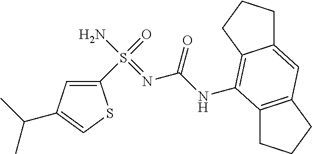 [SMILES: CC(C)c1csc(S(N)(=O)=NC(=O)Nc2c3c(cc4c2CCC4)CCC3)c1] |
| 379a | 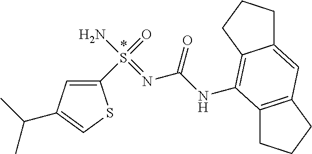 |
| 379b | 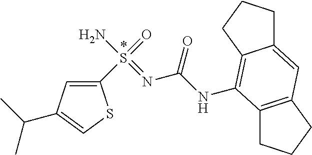 |
| 380 | 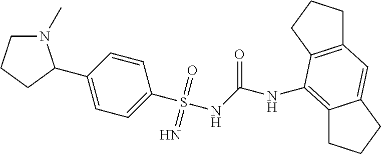 [SMILES: CN1CCCC1c1ccc(S(=N)(=O)NC(=O)Nc2c3c(cc4c2CCC4)CCC3)cc1] |
| 380a | 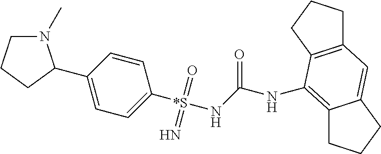 |
| 380b | 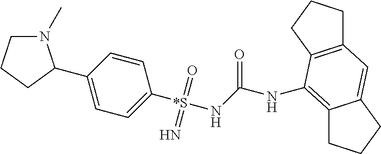 |
| 380c | 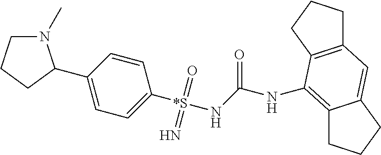 |
| 380d | 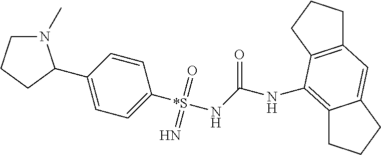 |
| 382 | 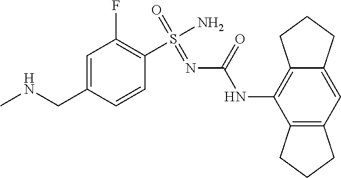 [SMILES: CNCc1ccc(S(N)(=O)=NC(=O)Nc2c3c(cc4c2CCC4)CCC3)c(F)c1] |
| 382a | 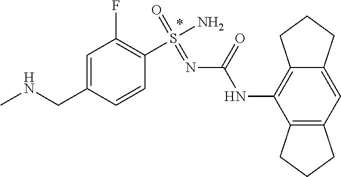 [SMILES: CNCc1ccc(S(N)(=O)=NC(=O)Nc2c3c(cc4c2CCC4)CCC3)c(F)c1] |
| 382b | 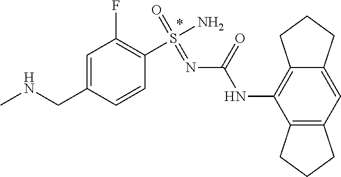 [SMILES: CNCc1ccc(S(N)(=O)=NC(=O)Nc2c3c(cc4c2CCC4)CCC3)c(F)c1] |
| 383 | 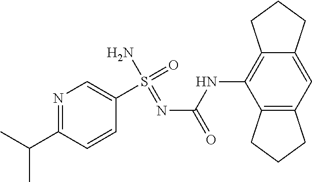 [SMILES: CC(C)c1ccc(S(N)(=O)=NC(=O)Nc2c3c(cc4c2CCC4)CCC3)cn1] |
| 383a | 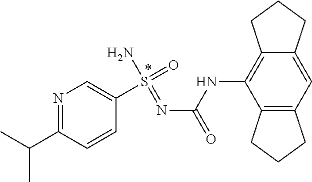 [SMILES: CC(C)c1ccc(S(N)(=O)=NC(=O)Nc2c3c(cc4c2CCC4)CCC3)cn1] |
| 383b | 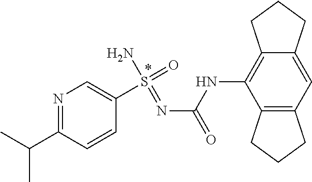 [SMILES: CC(C)c1ccc(S(N)(=O)=NC(=O)Nc2c3c(cc4c2CCC4)CCC3)cn1] |
| 384a | 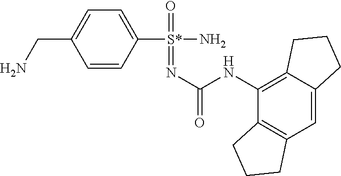 |
| 384b | 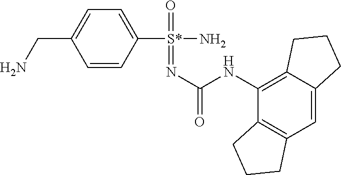 |
| 387a | 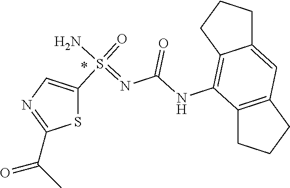 |
| 387b | 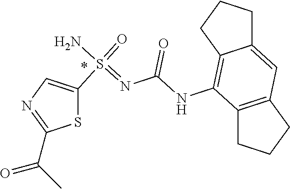 |

and pharmaceutically acceptable salts thereof.

In another aspect, an NLRP3 antagonist is a compound selected from the group consisting of the compounds in Table 14 below:

TABLE 14

| | |
|---|---|
| 401 | 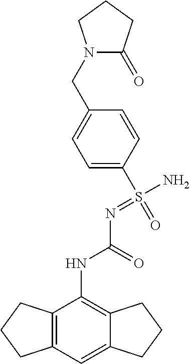 [SMILES: NS(=O)(=NC(=O)Nc1c2c(cc3c1CCC3)CCC2)c1ccc(CN2CCCC2=O)cc1] |
| 402 | 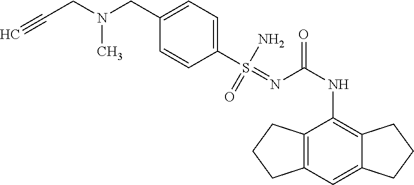 [SMILES: C#CCN(C)Cc1ccc(S(N)(=O)=NC(=O)Nc2c3c(cc4c2CCC4)CCC3)cc1] |
| 403 | 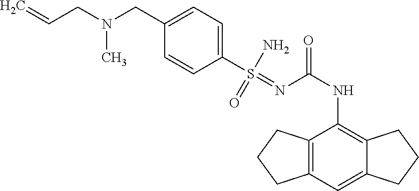 [SMILES: C=CCN(C)Cc1ccc(S(N)(=O)=NC(=O)Nc2c3c(cc4c2CCC4)CCC3)cc1] |
| 404 | 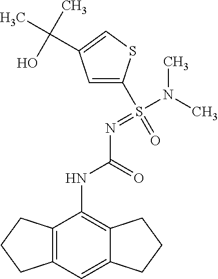 [SMILES: CN(C)S(=O)(=NC(=O)Nc1c2c(cc3c1CCC3)CCC2)c1cc(C(C)(C)O)cs1] |
| 404a | 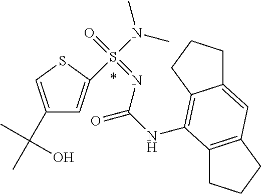 |
| 404b | 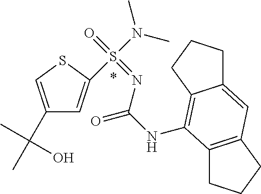 |
| 405 | 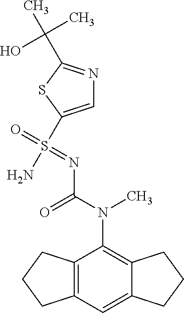 [SMILES: CN(C(=O)N=S(N)(=O)c1cnc(C(C)(C)O)s1)c1c2c(cc3c1CCC3)CCC2] |
| 406 | 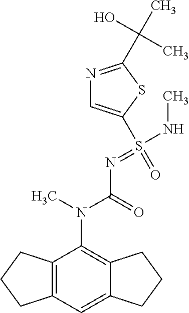 [SMILES: CNS(=O)(=NC(=O)N(C)c1c2c(cc3c1CCC3)CCC2)c1cnc(C(C)(C)O)s1] |
| 407 | 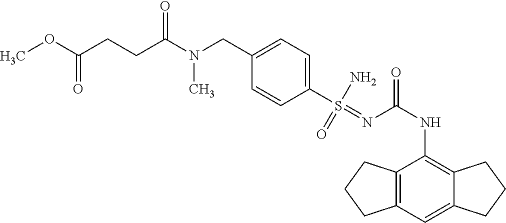 [SMILES: COC(=O)CCC(=O)N(C)Cc1ccc(S(N)(=O)=NC(=O)Nc2c3c(cc4c2CCC4)CCC3)cc1] |
| 408 | 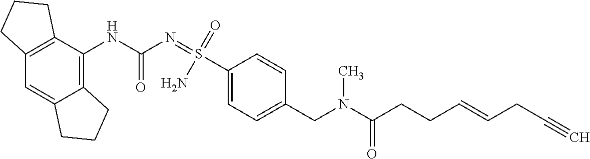 [SMILES: C#CC/C=C/CCC(=O)N(C)Cc1ccc(S(N)(=O)=NC(=O)Nc2c3c(cc4c2CCC4)CCC3)cc1] |
| 409 | 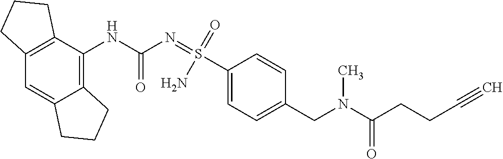 [SMILES: C#CCCC(=O)N(C)Cc1ccc(S(N)(=O)=NC(=O)Nc2c3c(cc4c2CCC4)CCC3)cc1] |
| 409a | 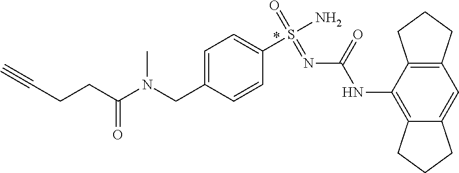 [SMILES: C#CCCC(=O)N(C)Cc1ccc(S(N)(=O)=NC(=O)Nc2c3c(cc4c2CCC4)CCC3)cc1] |
| 409b | 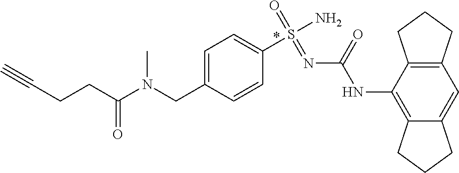 [SMILES: C#CCCC(=O)N(C)Cc1ccc(S(N)(=O)=NC(=O)Nc2c3c(cc4c2CCC4)CCC3)cc1] |
| 410 | 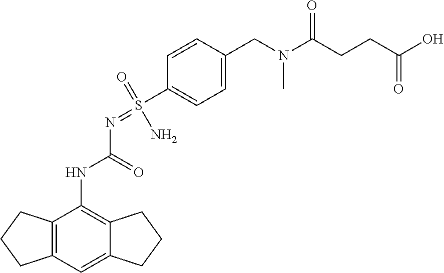 [SMILES: CN(Cc1ccc(S(N)(=O)=NC(=O)Nc2c3c(cc4c2CCC4)CCC3)cc1)C(=O)CCC(=O)O] |

and pharmaceutically acceptable salts thereof.

In some embodiments of the compound of Formula XI, the sulfur in the moiety S(═O)(NHR³)═N— has (S) stereochemistry.

In some embodiments of the methods described herein, the compounds of Formula XI and compounds listed in Tables 11-14 above, are administered in combination with an anti-TNFα agent; preferably in combination with Infliximab, Etanercept, Certolizumab pegol, Golimumab or Adalimumab; more preferably in combination with Adalimumab; more preferably in combination with Adalimumab.

In some embodiments of the compound of Formula XI, the sulfur in the moiety S(═O)(NHR³)═N— has (R) stereochemistry.

Compounds having Formula XI, as well as methods of making and using the same, are further described in PCT Application No. PCT/US2018/043338, entitled “Compounds And Compositions For Treating Conditions Associated With NLRP Activity”, filed on Jul. 23, 2018, which is incorporated herein by reference in its entirety.

In one aspect, an NLRP3 antagonist is a compound of Formula XII

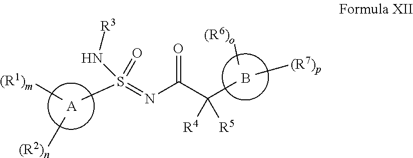

wherein
m=0, 1, or 2;
n=0, 1, or 2;
o=1 or 2;
p=0, 1, 2, or 3;
wherein
A is a 5-10-membered monocyclic or bicyclic heteroaryl or a C₆-C₁₀ monocyclic or bicyclic aryl;
B is a 5-10-membered monocyclic or bicyclic heteroaryl or a C₆-C₁₀ monocyclic or bicyclic aryl;
wherein
at least one R⁶ is ortho to the bond connecting the B ring to the C(R⁴R⁵) group of Formula XII;
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO—C₆-C₁₀ aryl, CO-5- to 10-membered heteroaryl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), and NHCOC₂-C₆ alkynyl;
   wherein each C₁-C₆ alkyl substituent and each C₁-C₆ alkoxy substituent of the R¹ or R² C₃-C₇ cycloalkyl or of the R¹ or R² 3-to 7-membered heterocycloalkyl is further optionally independently substituted with one to three hydroxy, halo, NR⁸R⁹, or oxo;
   wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;
or at least one pair of R¹ and R² on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, NR¹⁰ COOC₁-C₆ alkyl, C₆-C₁₀ aryl, and CONR⁸R^{9′} wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹; R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl, wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl, or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
   wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5-to-8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰ COOC₁-C₆ alkyl, C₆-C₁₀ aryl, and CONR⁸R⁹;
each of R⁴ and R⁵ is independently selected from hydrogen and C₁-C₆ alkyl;
R₁₀ is C₁-C₆ alkyl;
each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl;
R³ is selected from hydrogen, cyano, hydroxy, C₁-C₆ alkoxy, C₁-C₆ alkyl, and

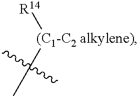

wherein the C₁-C₂ alkylene group is optionally substituted by oxo; R¹⁴ is hydrogen, C₁-C₆ alkyl, 5-10-membered monocyclic or bicyclic heteroaryl or C₆-C₁₀ monocyclic or bicyclic aryl, wherein each C₁-C₆ alkyl, aryl or heteroaryl is optionally independently substituted with 1 or 2 R⁶,
or a pharmaceutically acceptable salt thereof.

In some embodiments of the compound of Formula XII, A is a 5-6-membered monocyclic heteroaryl optionally substituted with 1 or 2 R¹ and optionally substituted with 1 or 2 R².

In some embodiments of the compound of Formula XII, A is furanyl optionally substituted with 1 or 2 R¹ and optionally substituted with 1 or 2 R².

In some embodiments of the compound of Formula XII, A is thiophenyl optionally substituted with 1 or 2 R¹ and optionally substituted with 1 or 2 R².

In some embodiments of the compound of Formula XII, A is oxazolyl optionally substituted with 1 or 2 R¹ and optionally substituted with 1 or 2 R².

In some embodiments of the compound of Formula XII, A is thiazolyl optionally substituted with 1 or 2 R¹ and optionally substituted with 1 or 2 R².

In some embodiments of the compound of Formula XII, A is phenyl optionally substituted with 1 or 2 R¹ and optionally substituted with 1 or 2 R².

In some embodiments of the compound of Formula XII, m=1 and n=0.

In some embodiments of the compound of Formula XII, the substituted ring A is

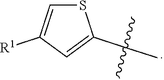

In some embodiments of the compound of Formula XII, the substituted ring A is

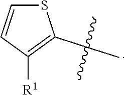

In some embodiments of the compound of Formula XII, the substituted ring A is

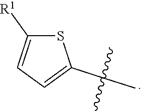

In some embodiments of the compound of Formula XII, the substituted ring A is

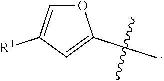

In some embodiments of the compound of Formula XII, the substituted ring A is

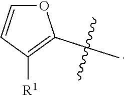

In some embodiments of the compound of Formula XII, the substituted ring A is

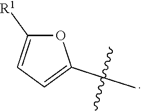

In some embodiments of the compound of Formula XII, the substituted ring A is

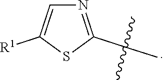

In some embodiments of the compound of Formula XII, the substituted ring A is

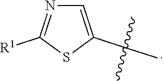

In some embodiments of the compound of Formula XII, the substituted ring A is

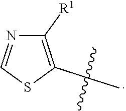

In some embodiments of the compound of Formula XII, the substituted ring A is

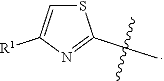

In some embodiments of the compound of Formula XII, the substituted ring A is

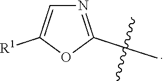

In some embodiments of the compound of Formula XII, the substituted ring A is

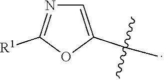

In some embodiments of the compound of Formula XII, the substituted ring A is

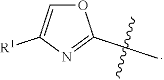

In some embodiments of the compound of Formula XII, the substituted ring A is

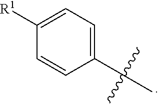

In some embodiments of the compound of Formula XII, the substituted ring A is

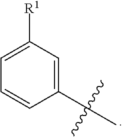

In some embodiments of the compound of Formula XII, the substituted ring A is

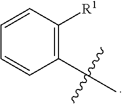

In some embodiments of the compound of Formula XII, the substituted ring A is

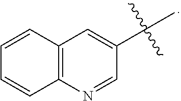 [SMILES: *c1cnc2ccccc2c1]

In some embodiments of the compound of Formula XII, the substituted ring A is

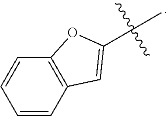 [SMILES: *c1cc2ccccc2o1]

In some embodiments of the compound of Formula XII, the substituted ring A is

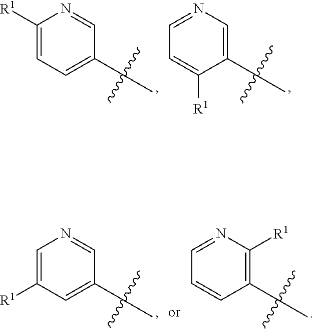

In some embodiments of the compound of Formula XII, the substituted ring A is

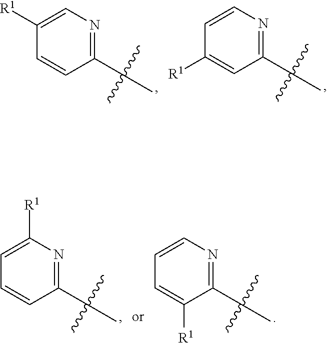

In some embodiments of the compound of Formula XII, the substituted ring A is

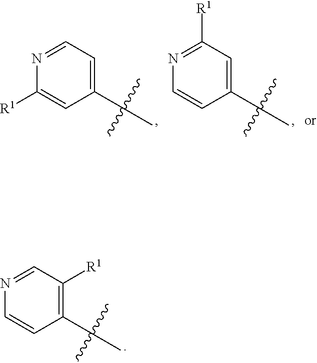

In some embodiments of the compound of Formula XII, m=1 and n=1.

In some embodiments of the compound of Formula XII, the substituted ring A is

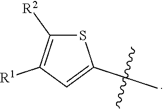

In some embodiments of the compound of Formula XII, the substituted ring A is

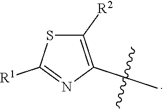

In some embodiments of the compound of Formula XII, the substituted ring A is

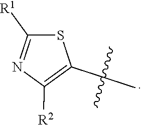

In some embodiments of the compound of Formula XII, the substituted ring A is

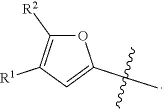

In some embodiments of the compound of Formula XII, the substituted ring A is

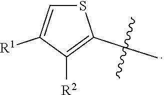

In some embodiments of the compound of Formula XII, the substituted ring A is

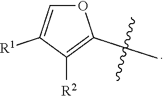

In some embodiments of the compound of Formula XII, the substituted ring A is

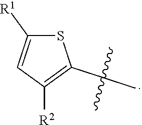

In some embodiments of the compound of Formula XII, the substituted ring A is

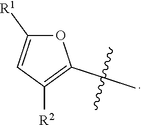

In some embodiments of the compound of Formula XII, the substituted ring A is

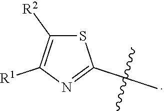

In some embodiments of the compound of Formula XII, the substituted ring A is

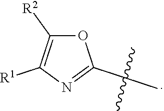

In some embodiments of the compound of Formula XII, the substituted ring A is

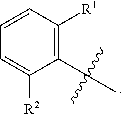

In some embodiments of the compound of Formula XII, the substituted ring A is

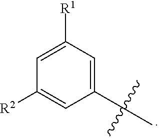

In some embodiments of the compound of Formula XII, the substituted ring A is

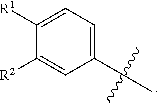

In some embodiments of the compound of Formula XII, the substituted ring A is

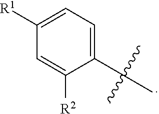

In some embodiments of the compound of Formula XII, the substituted ring A is

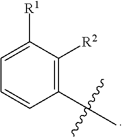

In some embodiments of the compound of Formula XII, the substituted ring A is

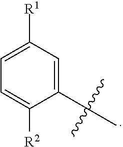

In some embodiments of the compound of Formula XII, m=2 and n=1.

In some embodiments of the compound of Formula XII, the substituted ring A is

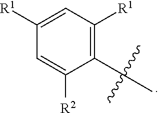

In some embodiments of the compound of Formula XII, the substituted ring A is

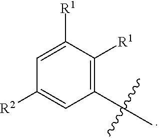

In some embodiments of the compound of Formula XII, the substituted ring A is

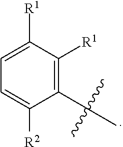

In some embodiments of the compound of Formula XII, the substituted ring A is

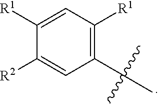

In some embodiments of the compound of Formula XII, each of R¹ and R², when present, is independently selected from the group consisting of C₁-C₆ alkyl optionally substituted with one or more hydroxy, halo, oxo, C₁-C₆ alkoxy, or NR⁸R⁹; C₃-C₇ cycloalkyl optionally substituted with one or more hydroxy, halo, oxo, C₁-C₆ alkoxy, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; 3- to 7-membered heterocycloalkyl optionally substituted with one or more hydroxy, halo, oxo, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; C₁-C₆ haloalkyl; C₁-C₆ alkoxy; C₁-C₆ haloalkoxy; halo; CN; CO—C₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO (5- to 10-membered heteroaryl); OCO (3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; S(O₂)NR¹¹R¹²; S(O)C₁-C₆ alkyl; and S(O₂)C₁-C₆ alkyl.

In some embodiments of the compound of Formula XII, R¹ is selected from the group consisting of 1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; S(O₂)CH₃, and S(O₂)NR¹¹R¹².

In certain embodiments (when each of R¹ and R², when present, is independently selected from the group consisting of C₁-C₆ alkyl optionally substituted with one or more hydroxy, halo, oxo, C₁-C₆ alkoxy, or NR⁸R⁹; C₃-C₇ cycloalkyl optionally substituted with one or more hydroxy, halo, oxo, C₁-C₆ alkoxy, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; 3- to 7-membered heterocycloalkyl optionally substituted with one or more hydroxy, halo, oxo, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; C₁-C₆ haloalkyl; C₁-C₆ alkoxy; C₁-C₆ haloalkoxy; halo; CN; CO—C₁-C₆ alkyl; CO—C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO (5- to 10-membered heteroaryl); OCO (3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; S(O₂)NR¹¹R¹²; S(O)C₁-C₆ alkyl; and S(O₂)C₁-C₆ alkyl), R² is selected from the group consisting of fluoro; chloro; cyano; methyl; methoxy; ethoxy; isopropyl; 1-hydroxy-2-methylpropan-2-yl; 2-hydroxy-2-propyl; hydroxymethyl; 1-hydroxyethyl; 2-hydroxyethyl; 1-hydroxy-2-propyl; 1-hydroxy-1-cyclopropyl; COCH₃; COPh; 2-methoxy-2-propyl; (dimethylamino)methyl; S(O₂)CH₃, and S(O₂)NR¹¹R¹².

In some embodiments of the compound of Formula XII, B is phenyl substituted with 1 or 2 R⁶ and optionally substituted with 1, 2, or 3 R⁷.

In certain embodiments (when B is phenyl substituted with 1 or 2 R⁶ and optionally substituted with 1, 2, or 3 R⁷), o=2 and p=0.

In certain embodiments (when B is phenyl substituted with 1 or 2 R⁶ and optionally substituted with 1, 2, or 3 R⁷; and/or o=2 and p=0), the substituted ring B is

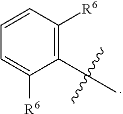

In certain embodiments (when the substituted ring B is

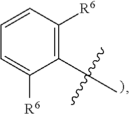

each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, ═NR¹⁰ COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl.

In certain embodiments (when the substituted ring B is

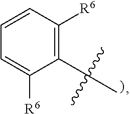

each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, and oxo.

In certain embodiments (when B is phenyl substituted with 1 or 2 R⁶ and optionally substituted with 1, 2, or 3 R⁷), o=1 and p=1.

In certain embodiments (when B is phenyl substituted with 1 or 2 R⁶ and optionally substituted with 1, 2, or 3 R⁷), o=2 and p=1.

In certain embodiments (when B is phenyl substituted with 1 or 2 R⁶ and optionally substituted with 1, 2, or 3 R⁷; and o=2 and p=1), the substituted ring B is

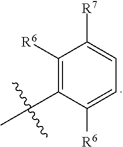

In certain embodiments (when the substituted ring B is

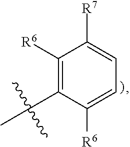

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷ on adjacent atoms, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain embodiments (when B is phenyl substituted with 1 or 2 R⁶ and optionally substituted with 1, 2, or 3 R⁷; and o=2 and p=1), the substituted ring B is

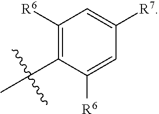

In certain embodiments (when the substituted ring B is

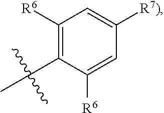

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

In certain embodiments (when B is phenyl substituted with 1 or 2 R⁶ and optionally substituted with 1, 2, or 3 R⁷), o=2 and p=2.

In certain embodiments (when B is phenyl substituted with 1 or 2 R⁶ and optionally substituted with 1, 2, or 3 R⁷; and o=2 and p=2), the substituted ring B is

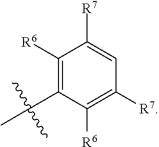

In certain embodiments (when the substituted ring B is

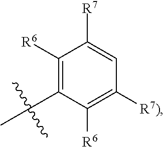

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain embodiments (when B is phenyl substituted with 1 or 2 R⁶ and optionally substituted with 1, 2, or 3 R⁷; and o=2 and p=2), the substituted ring B is

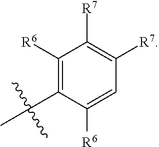

In certain embodiments (when the substituted ring B is

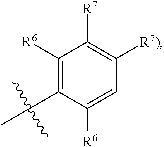

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R⁶ and R⁷ on adjacent atoms, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain embodiments (when B is phenyl substituted with 1 or 2 R⁶ and optionally substituted with 1, 2, or 3 R⁷), o=2 and p=3

In certain embodiments (when B is phenyl substituted with 1 or 2 R⁶ and optionally substituted with 1, 2, or 3 R⁷; and o=2 and p=3), the substituted ring B is

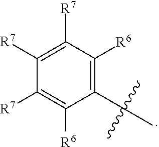

In certain embodiments (when the substituted ring B is

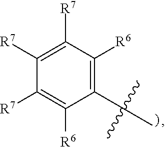

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰ COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰ COOC₁-C₆ alkyl, C₆-C₁₀ aryl, and CONR⁸R⁹.

In some embodiments of the compound of Formula XII, B is pyridyl; and o=1 or 2 and p=0, 1, or 2.

In certain embodiments (when B is pyridyl; and o=1 or 2 and p=0, 1, or 2), o=2 and p=1.

In certain embodiments (when B is pyridyl; and o=2 and p=1), the substituted ring B is

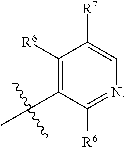

In certain embodiments (when the substituted ring B is

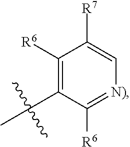

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₆-C₁₀ aryl is optionally substituted with one to two C₁-C₆ alkyl optionally substituted with one to three halo;

or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰ COOC₁-C₆ alkyl, C₆-C₁₀ aryl, and CONR⁸R⁹.

In certain embodiments (when B is pyridyl with o=1 or 2 and p=0, 1, or 2), o=2 and p=2.

In certain embodiments (when B is pyridyl; and o=2 and p=2), the substituted ring B is

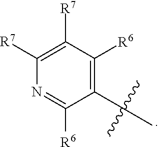

In certain embodiments (when the substituted ring B is

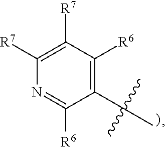

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO—C₁-C₆ alkyl, CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (4- to 6-membered heterocycloalkyl), NHCOC₁-C₆ alkyl, NHCOC₆-C₁₀ aryl, NHCO (5- to 10-membered heteroaryl), NHCO (4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₆-C₁₀ aryl is optionally substituted with one to two C₁-C₆ alkyl optionally substituted with one to three halo;

or R⁶ and R⁷, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, ═NR¹⁰ COOC₁-C₆ alkyl, C₆-C₁₀ aryl, and CONR⁸R⁹.

In some embodiments of the compound of Formula XII, each of R⁴ and R⁵ is hydrogen.

In some embodiments of the compound of Formula XII, R³ is hydrogen.

In another aspect, an NLRP3 antagonist is selected from the group consisting of the compounds in Table 15 below:

TABLE 15

| Com- | |
|---|---|
| pound | Structure |
| 101 | 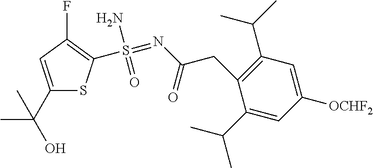 [SMILES: CC(C)c1cc(OC(F)F)cc(C(C)C)c1CC(=O)N=S(N)(=O)c1sc(C(C)(C)O)cc1F] |
| 101a | 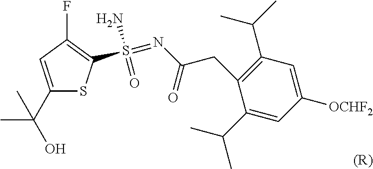 [SMILES: CC(C)c1cc(OC(F)F)cc(C(C)C)c1CC(=O)N=[S@@](N)(=O)c1sc(C(C)(C)O)cc1F ; "(R)"] |
| 101b | 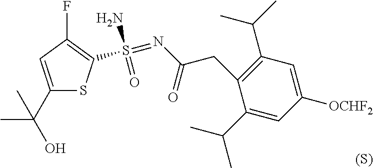 [SMILES: CC(C)c1cc(OC(F)F)cc(C(C)C)c1CC(=O)N=[S@](N)(=O)c1sc(C(C)(C)O)cc1F ; "(S)"] |
| 102 | 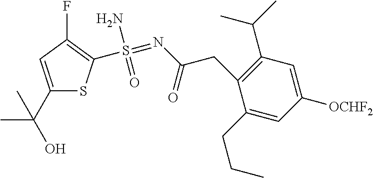 [SMILES: CCCc1cc(OC(F)F)cc(C(C)C)c1CC(=O)N=S(N)(=O)c1sc(C(C)(C)O)cc1F] |
| 103 | 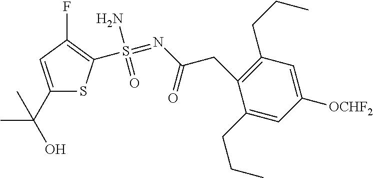 [SMILES: CCCc1cc(OC(F)F)cc(CCC)c1CC(=O)N=S(N)(=O)c1sc(C(C)(C)O)cc1F] |
| 104 | 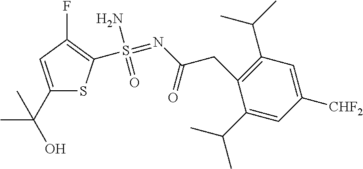 [SMILES: CC(C)c1cc(C(F)F)cc(C(C)C)c1CC(=O)N=S(N)(=O)c1sc(C(C)(C)O)cc1F] |
| 104a | 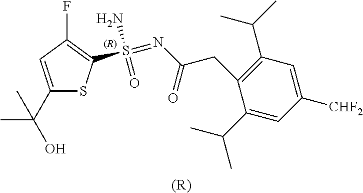 |
| 104b | 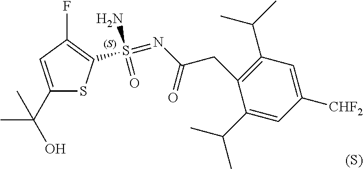 |
| 105 | 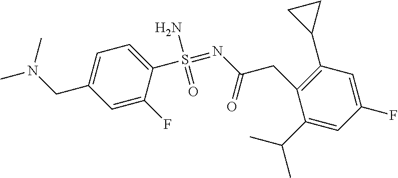 [SMILES: CC(C)c1cc(F)cc(C2CC2)c1CC(=O)N=S(N)(=O)c1ccc(CN(C)C)cc1F] |
| 106 | 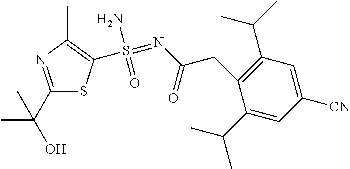 [SMILES: Cc1nc(C(C)(C)O)sc1S(N)(=O)=NC(=O)Cc1c(C(C)C)cc(C#N)cc1C(C)C] |
| 106a | 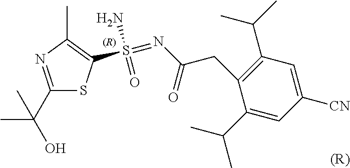 |
| 106b | 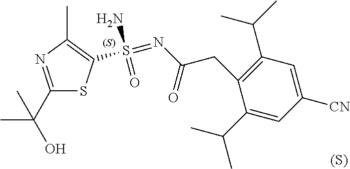 |
| 107 | 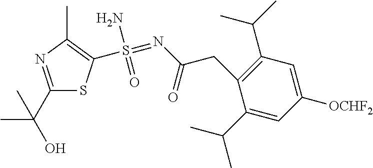 [SMILES: Cc1nc(C(C)(C)O)sc1S(N)(=O)=NC(=O)Cc1c(C(C)C)cc(OC(F)F)cc1C(C)C] |
| 107a | 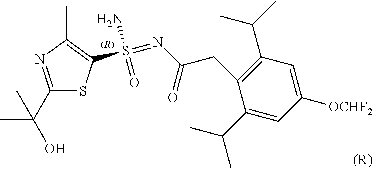 |
| 107b | 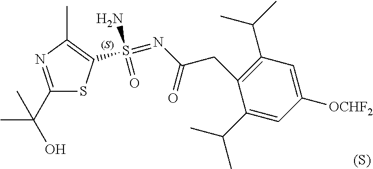 |
| 108 | 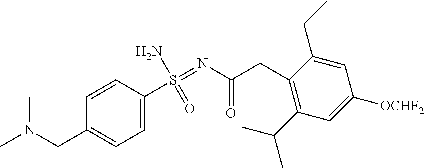 [SMILES: CCc1cc(OC(F)F)cc(C(C)C)c1CC(=O)N=S(N)(=O)c1ccc(CN(C)C)cc1] |
| 109 | 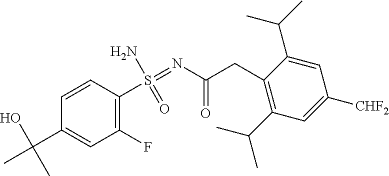 [SMILES: CC(C)c1cc(C(F)F)cc(C(C)C)c1CC(=O)N=S(N)(=O)c1ccc(C(C)(C)O)cc1F] |
| 110 | 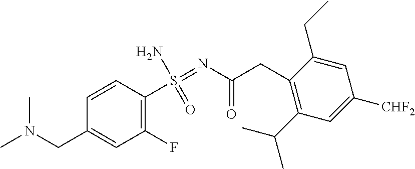 [SMILES: CCc1cc(C(F)F)cc(C(C)C)c1CC(=O)N=S(N)(=O)c1ccc(CN(C)C)cc1F] |
| 110a′ | 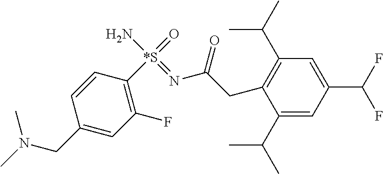 |
| 110b′ | 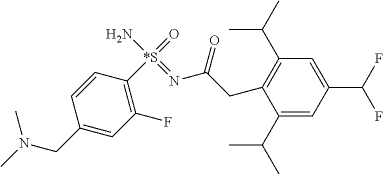 |
| 110a | 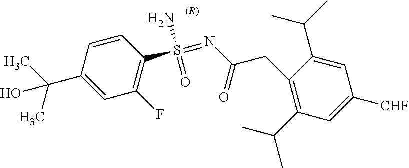 |
| 110b | 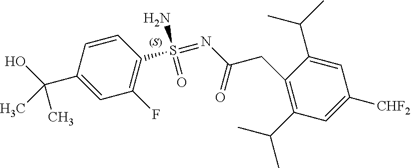 |
| 111 | 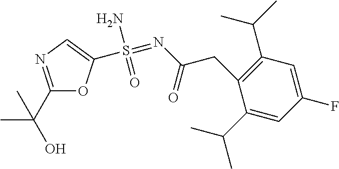 [SMILES: CC(C)c1cc(F)cc(C(C)C)c1CC(=O)N=S(N)(=O)c1cnc(C(C)(C)O)o1] |
| 112 | 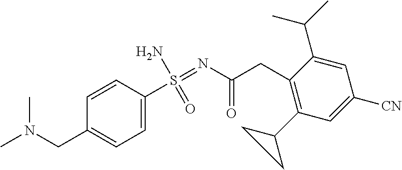 [SMILES: CC(C)c1cc(C#N)cc(C2CC2)c1CC(=O)N=S(N)(=O)c1ccc(CN(C)C)cc1] |
| 112a | 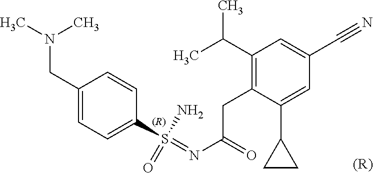 |
| 112b | 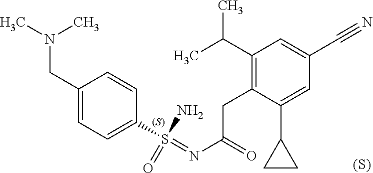 |
| 113 | 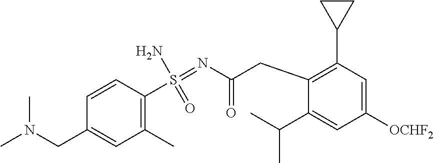 [SMILES: Cc1cc(CN(C)C)ccc1S(N)(=O)=NC(=O)Cc1c(C(C)C)cc(OC(F)F)cc1C1CC1] |
| 114 | 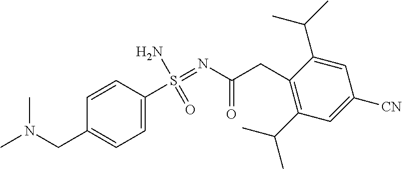 [SMILES: CC(C)c1cc(C#N)cc(C(C)C)c1CC(=O)N=S(N)(=O)c1ccc(CN(C)C)cc1] |
| 114a | 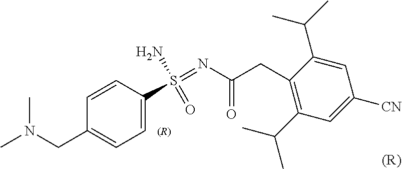 |
| 114b | 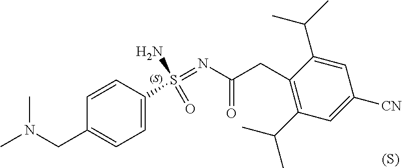 |
| 115 | 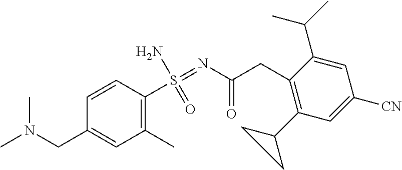 [SMILES: Cc1cc(CN(C)C)ccc1S(N)(=O)=NC(=O)Cc1c(C(C)C)cc(C#N)cc1C1CC1] |
| 116 | 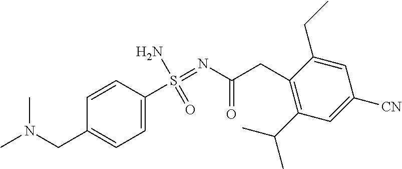 [SMILES: CCc1cc(C#N)cc(C(C)C)c1CC(=O)N=S(N)(=O)c1ccc(CN(C)C)cc1] |
| 116a | 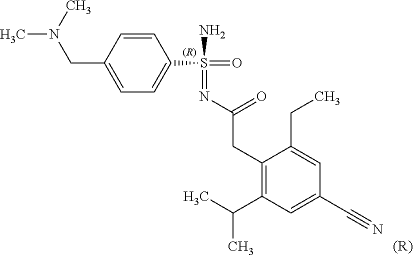 |
| 116b | 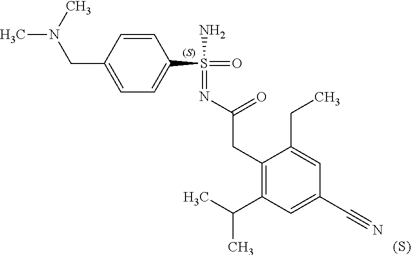 |
| 117 | 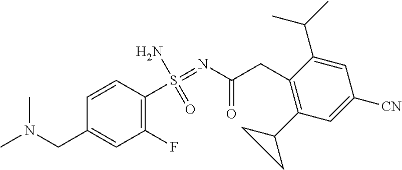 [SMILES: CC(C)c1cc(C#N)cc(C2CC2)c1CC(=O)N=S(N)(=O)c1ccc(CN(C)C)cc1F] |
| 117a | 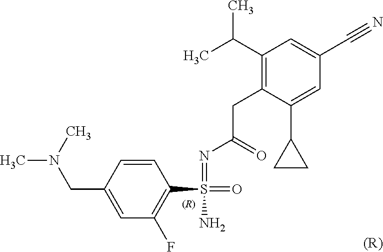 |
| 117b | 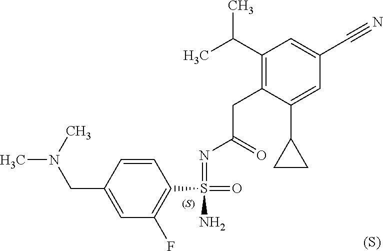 |
| 118 | 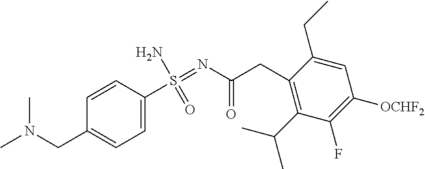 [SMILES: CCc1cc(OC(F)F)c(F)c(C(C)C)c1CC(=O)N=S(N)(=O)c1ccc(CN(C)C)cc1] |
| 119 | 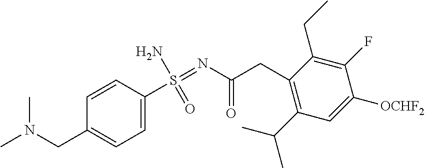 [SMILES: CCc1c(F)c(OC(F)F)cc(C(C)C)c1CC(=O)N=S(N)(=O)c1ccc(CN(C)C)cc1] |
| 120 | 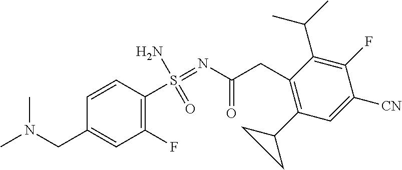 [SMILES: CC(C)c1c(F)c(C#N)cc(C2CC2)c1CC(=O)N=S(N)(=O)c1ccc(CN(C)C)cc1F] |
| 121 | 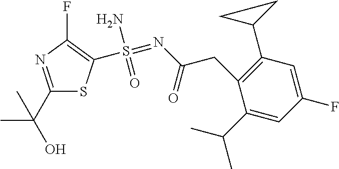 [SMILES: CC(C)c1cc(F)cc(C2CC2)c1CC(=O)N=S(N)(=O)c1sc(C(C)(C)O)nc1F] |
| 122 | 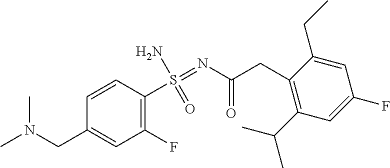 [SMILES: CCc1cc(F)cc(C(C)C)c1CC(=O)N=S(N)(=O)c1ccc(CN(C)C)cc1F] |
| 123 | 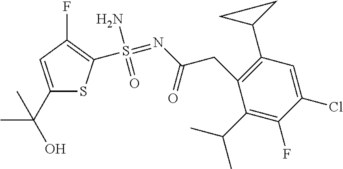 [SMILES: CC(C)c1c(F)c(Cl)cc(C2CC2)c1CC(=O)N=S(N)(=O)c1sc(C(C)(C)O)cc1F] |
| 123a | 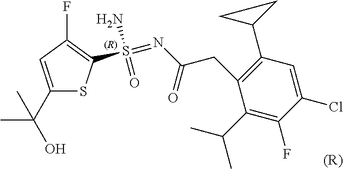 |
| 123b | 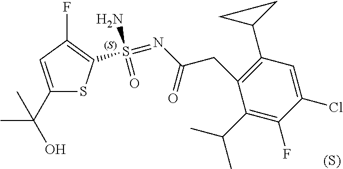 |
| 124 | 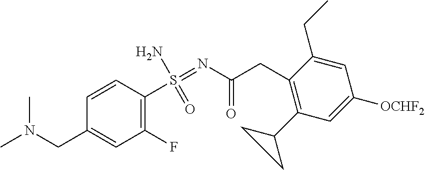 [SMILES: CCc1cc(OC(F)F)cc(C2CC2)c1CC(=O)N=S(N)(=O)c1ccc(CN(C)C)cc1F] |
| 125 | 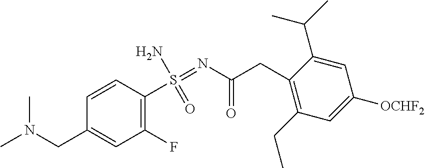 [SMILES: CCc1cc(OC(F)F)cc(C(C)C)c1CC(=O)N=S(N)(=O)c1ccc(CN(C)C)cc1F] |
| 126 | 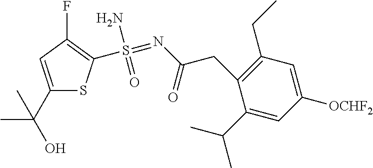 [SMILES: CCc1cc(OC(F)F)cc(C(C)C)c1CC(=O)N=S(N)(=O)c1sc(C(C)(C)O)cc1F] |
| 126a | 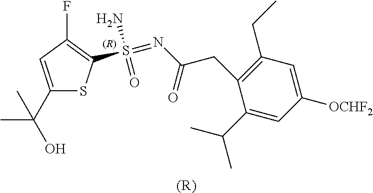 |
| 126b | 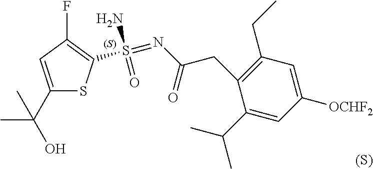 |
| 127 | 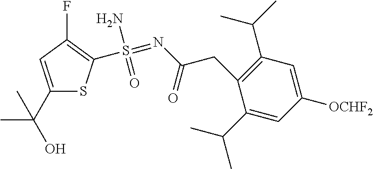 [SMILES: CC(C)c1cc(OC(F)F)cc(C(C)C)c1CC(=O)N=S(N)(=O)c1sc(C(C)(C)O)cc1F] |
| 128 | 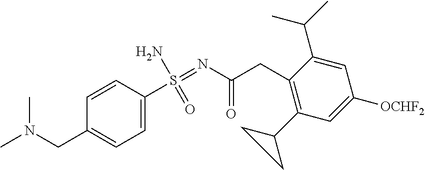 [SMILES: CC(C)c1cc(OC(F)F)cc(C2CC2)c1CC(=O)N=S(N)(=O)c1ccc(CN(C)C)cc1] |
| 129 | 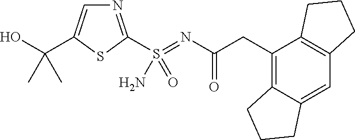 [SMILES: CC(C)(O)c1cnc(S(N)(=O)=NC(=O)Cc2c3c(cc4c2CCC4)CCC3)s1] |
| 129a | 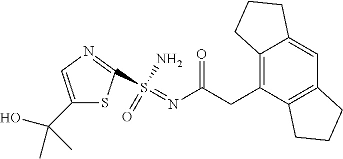 [SMILES: CC(C)(O)c1cnc([S@](N)(=O)=NC(=O)Cc2c3c(cc4c2CCC4)CCC3)s1] |
| 129b | 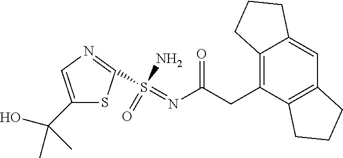 [SMILES: CC(C)(O)c1cnc([S@@](N)(=O)=NC(=O)Cc2c3c(cc4c2CCC4)CCC3)s1] |
| 130 | 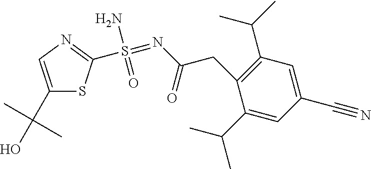 [SMILES: CC(C)c1cc(C#N)cc(C(C)C)c1CC(=O)N=S(N)(=O)c1ncc(C(C)(C)O)s1] |
| 130a | 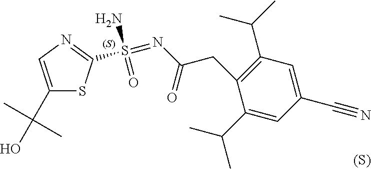 |
| 130b | 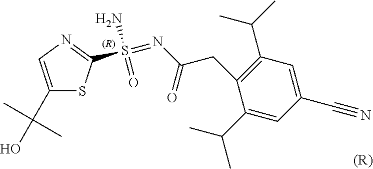 |
| 131 | 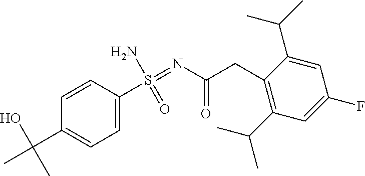 [SMILES: CC(C)c1cc(F)cc(C(C)C)c1CC(=O)N=S(N)(=O)c1ccc(C(C)(C)O)cc1] |
| 131a | 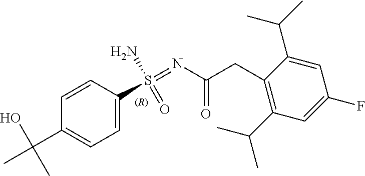 |
| 131b | 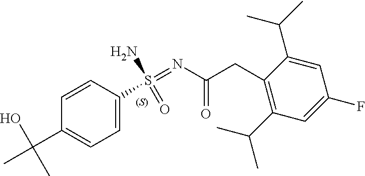 |
| 132 | 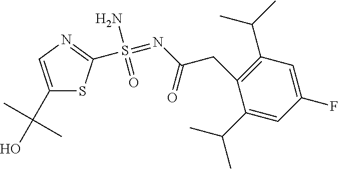 [SMILES: CC(C)c1cc(F)cc(C(C)C)c1CC(=O)N=S(N)(=O)c1ncc(C(C)(C)O)s1] |
| 132a | 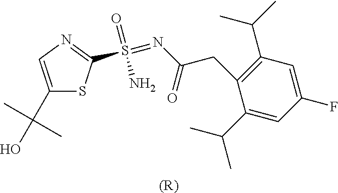 [SMILES: CC(C)c1cc(F)cc(C(C)C)c1CC(=O)N=[S@](N)(=O)c1ncc(C(C)(C)O)s1 ; "(R)"] |
| 132b | 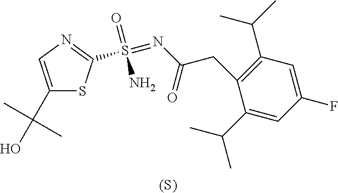 [SMILES: CC(C)c1cc(F)cc(C(C)C)c1CC(=O)N=[S@@](N)(=O)c1ncc(C(C)(C)O)s1 ; "(S)"] |
| 133 | 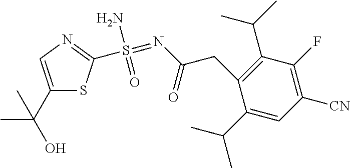 [SMILES: CC(C)c1cc(C#N)c(F)c(C(C)C)c1CC(=O)N=S(N)(=O)c1ncc(C(C)(C)O)s1] |
| 133a | 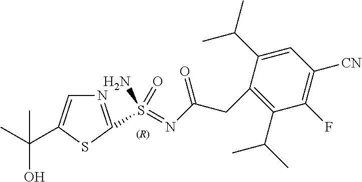 |
| 133b | 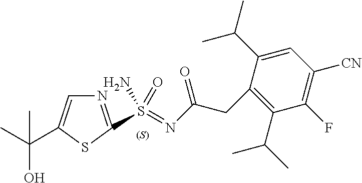 |
| 134 | 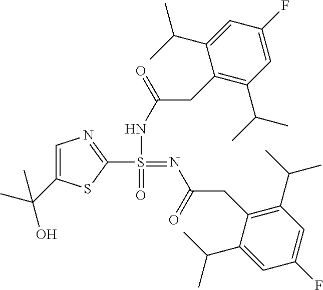 [SMILES: CC(C)c1cc(F)cc(C(C)C)c1CC(=O)N=S(=O)(NC(=O)Cc1c(C(C)C)cc(F)cc1C(C)C)c1ncc(C(C)(C)O)s1] |
| 135 | 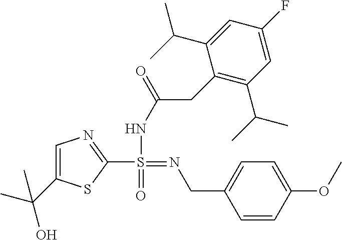 [SMILES: COc1ccc(CN=S(=O)(NC(=O)Cc2c(C(C)C)cc(F)cc2C(C)C)c2ncc(C(C)(C)O)s2)cc1] |
| 136 | 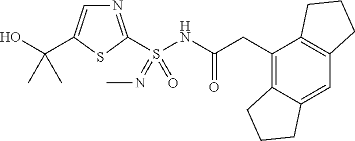 [SMILES: CN=S(=O)(NC(=O)Cc1c2c(cc3c1CCC3)CCC2)c1ncc(C(C)(C)O)s1] |
| 137 | 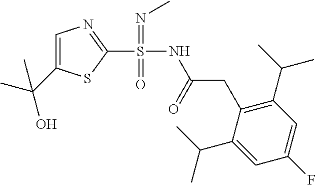 [SMILES: CN=S(=O)(NC(=O)Cc1c(C(C)C)cc(F)cc1C(C)C)c1ncc(C(C)(C)O)s1] |
| 138 | 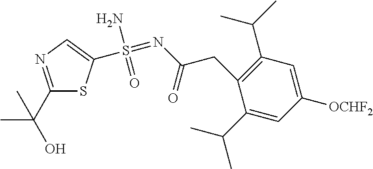 [SMILES: CC(C)c1cc(OC(F)F)cc(C(C)C)c1CC(=O)N=S(N)(=O)c1cnc(C(C)(C)O)s1] |
| 138a | 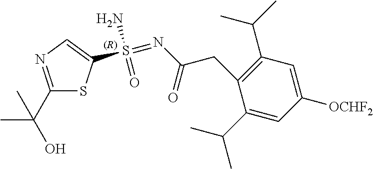 |
| 138b | 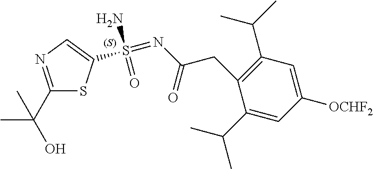 |
| 139 | 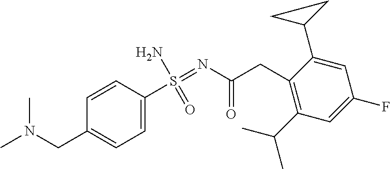 [SMILES: CC(C)c1cc(F)cc(C2CC2)c1CC(=O)N=S(N)(=O)c1ccc(CN(C)C)cc1] |
| 139a | 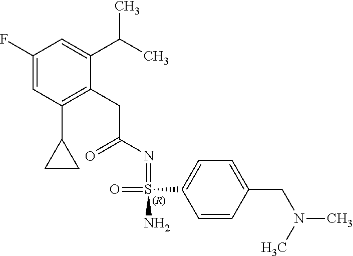 |
| 139b | 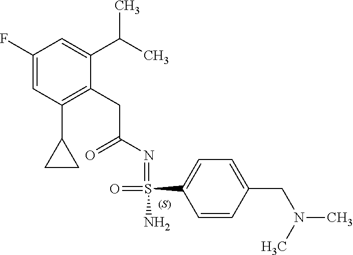 |
| 140 | 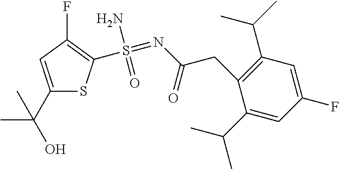 [SMILES: CC(C)c1cc(F)cc(C(C)C)c1CC(=O)N=S(N)(=O)c1sc(C(C)(C)O)cc1F] |
| 140a | 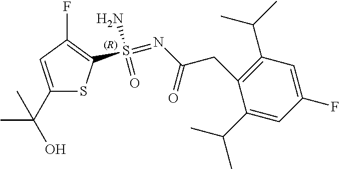 |
| 140b | 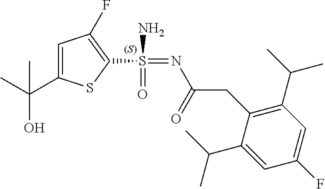 |
| 141 | 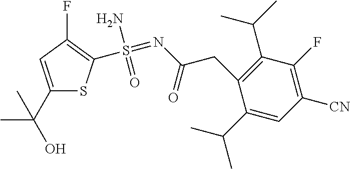 [SMILES: CC(C)c1cc(C#N)c(F)c(C(C)C)c1CC(=O)N=S(N)(=O)c1sc(C(C)(C)O)cc1F] |
| 141a | 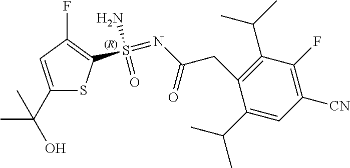 |
| 141b | 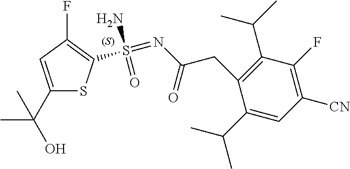 |
| 142 | 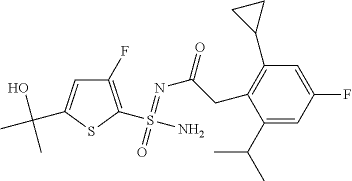 [SMILES: CC(C)c1cc(F)cc(C2CC2)c1CC(=O)N=S(N)(=O)c1sc(C(C)(C)O)cc1F] |
| 143 | 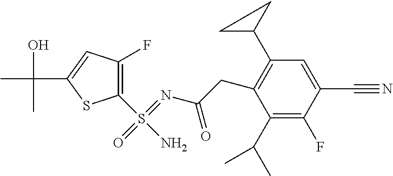 [SMILES: CC(C)c1c(F)c(C#N)cc(C2CC2)c1CC(=O)N=S(N)(=O)c1sc(C(C)(C)O)cc1F] |
| 144 | 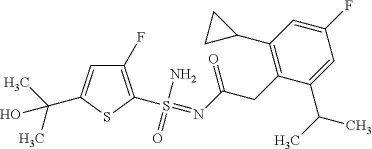 [SMILES: CC(C)c1cc(F)cc(C2CC2)c1CC(=O)N=S(N)(=O)c1sc(C(C)(C)O)cc1F] |
| 144a | 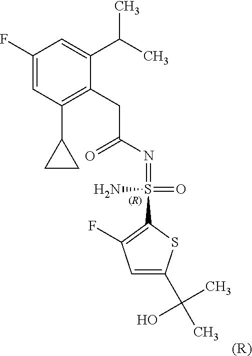 |
| 144b | 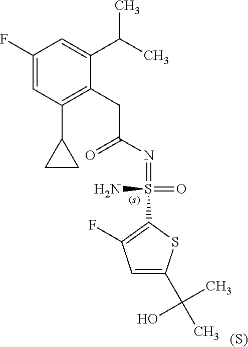 |
| 145 | 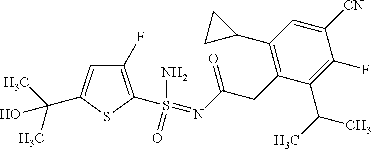 [SMILES: CC(C)c1c(F)c(C#N)cc(C2CC2)c1CC(=O)N=S(N)(=O)c1sc(C(C)(C)O)cc1F] |
| 145a | 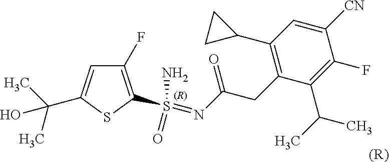 |
| 145b | 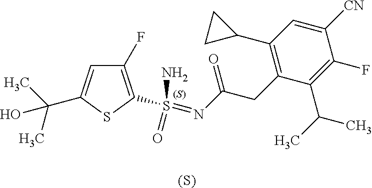 |
| 146 | 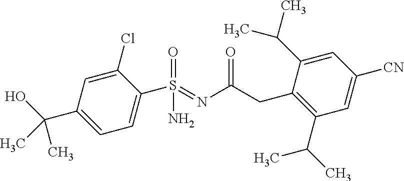 [SMILES: CC(C)c1cc(C#N)cc(C(C)C)c1CC(=O)N=S(N)(=O)c1ccc(C(C)(C)O)cc1Cl] |
| 147 | 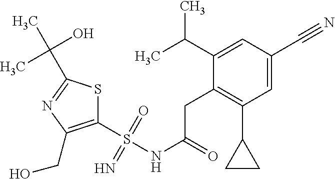 [SMILES: CC(C)c1cc(C#N)cc(C2CC2)c1CC(=O)NS(=N)(=O)c1sc(C(C)(C)O)nc1CO] |
| 147a | 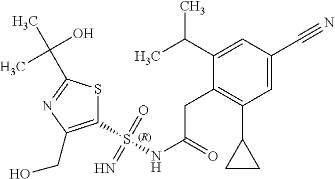 |
| 147b | 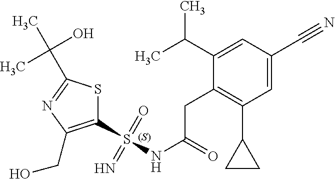 |
| 148 | 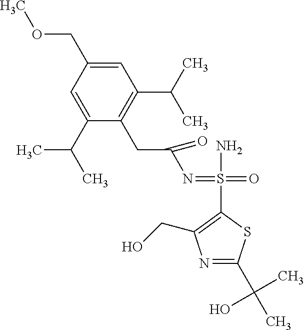 [SMILES: COCc1cc(C(C)C)c(CC(=O)N=S(N)(=O)c2sc(C(C)(C)O)nc2CO)c(C(C)C)c1] |
| 148a | |
| 148b | |
| 149a | |
| 149b | |
| 150 | 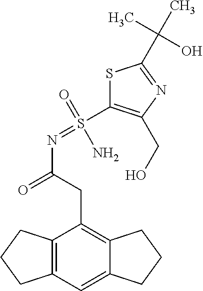 [SMILES: CC(C)(O)c1nc(CO)c(S(N)(=O)=NC(=O)Cc2c3c(cc4c2CCC4)CCC3)s1] |
| 150a | 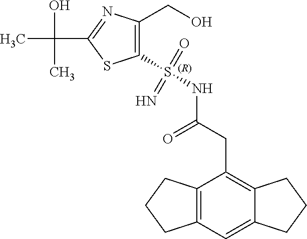 |
| 150b | 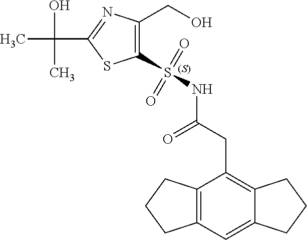 |
| 151 | |
| 152 | 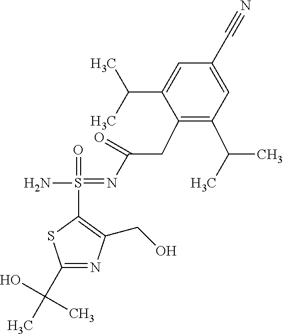 [SMILES: CC(C)c1cc(C#N)cc(C(C)C)c1CC(=O)N=S(N)(=O)c1sc(C(C)(C)O)nc1CO] |
| 152a | |
| 152b | |
| 153 | 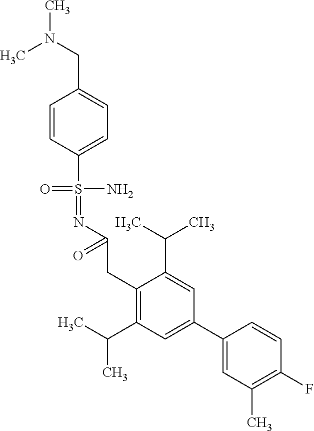 [SMILES: Cc1cc(-c2cc(C(C)C)c(CC(=O)N=S(N)(=O)c3ccc(CN(C)C)cc3)c(C(C)C)c2)ccc1F] |
| 154 | 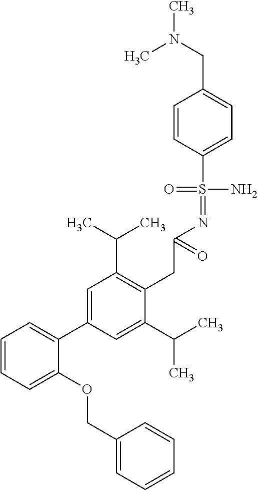 [SMILES: CC(C)c1cc(-c2ccccc2OCc2ccccc2)cc(C(C)C)c1CC(=O)N=S(N)(=O)c1ccc(CN(C)C)cc1] |
| 155 | 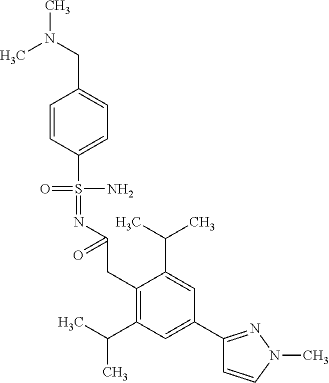 |
| 156 | 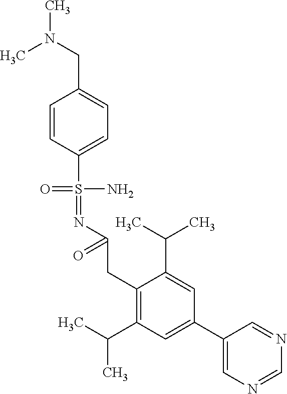 [SMILES: CC(C)c1cc(-c2cncnc2)cc(C(C)C)c1CC(=O)N=S(N)(=O)c1ccc(CN(C)C)cc1] |
| 157 | 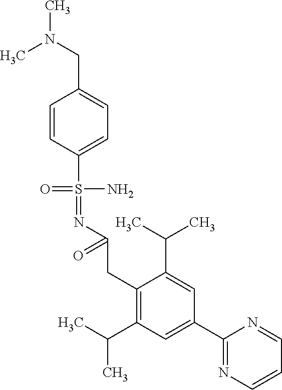 [SMILES: CC(C)c1cc(-c2ncccn2)cc(C(C)C)c1CC(=O)N=S(N)(=O)c1ccc(CN(C)C)cc1] |
| 158 | 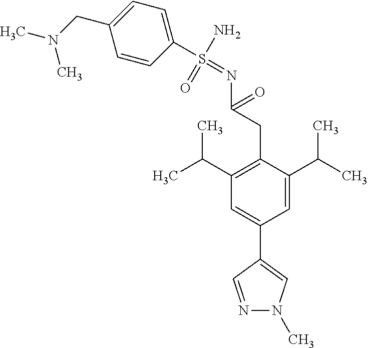 [SMILES: CC(C)c1cc(-c2cnn(C)c2)cc(C(C)C)c1CC(=O)N=S(N)(=O)c1ccc(CN(C)C)cc1] |
| 159 | 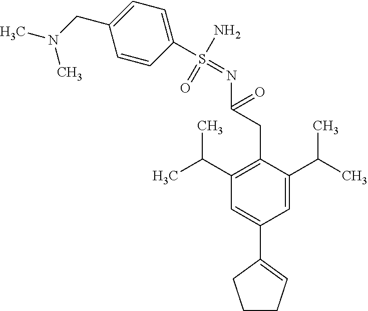 [SMILES: CC(C)c1cc(C2=CCCC2)cc(C(C)C)c1CC(=O)N=S(N)(=O)c1ccc(CN(C)C)cc1] |
| 160 | 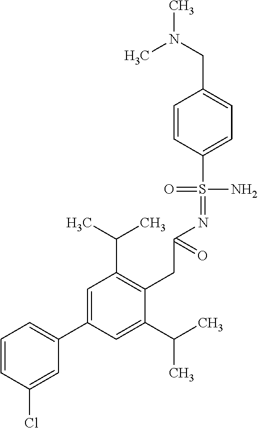 [SMILES: CC(C)c1cc(-c2cccc(Cl)c2)cc(C(C)C)c1CC(=O)N=S(N)(=O)c1ccc(CN(C)C)cc1] |
| 161 | 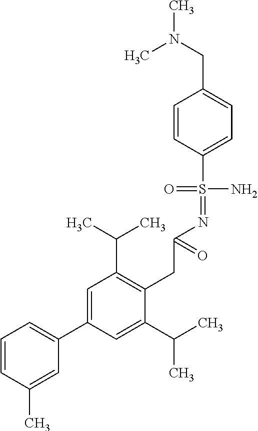 [SMILES: Cc1cccc(-c2cc(C(C)C)c(CC(=O)N=S(N)(=O)c3ccc(CN(C)C)cc3)c(C(C)C)c2)c1] |
| 162 | 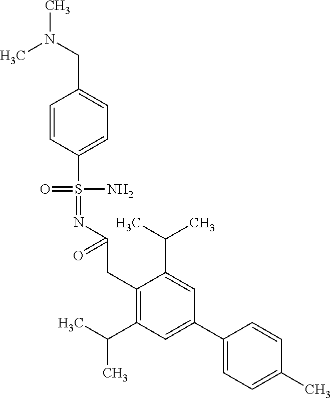 |
| 163 | 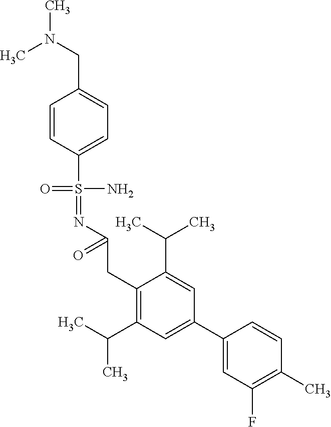 |
| 164 | 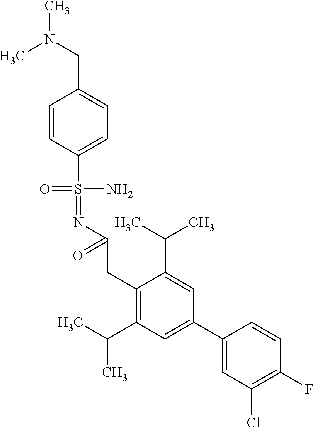 [SMILES: CC(C)c1cc(-c2ccc(F)c(Cl)c2)cc(C(C)C)c1CC(=O)N=S(N)(=O)c1ccc(CN(C)C)cc1] |
| 165 | 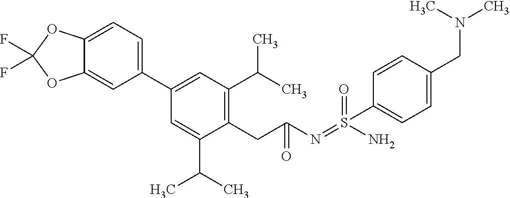 [SMILES: CC(C)c1cc(-c2ccc3c(c2)OC(F)(F)O3)cc(C(C)C)c1CC(=O)N=S(N)(=O)c1ccc(CN(C)C)cc1] |
| 166 | 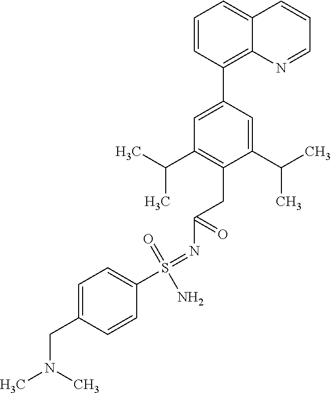 [SMILES: CC(C)c1cc(-c2cccc3cccnc23)cc(C(C)C)c1CC(=O)N=S(N)(=O)c1ccc(CN(C)C)cc1] |
| 167 | 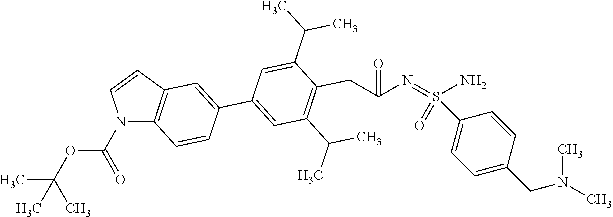 [SMILES: CC(C)c1cc(-c2ccc3c(ccn3C(=O)OC(C)(C)C)c2)cc(C(C)C)c1CC(=O)N=S(N)(=O)c1ccc(CN(C)C)cc1] |
| 168 | 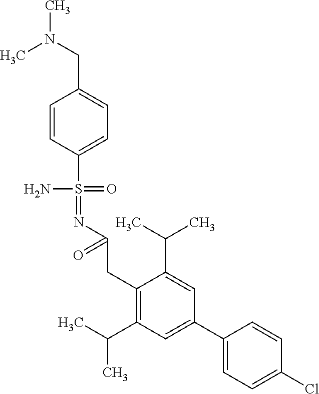 [SMILES: CC(C)c1cc(-c2ccc(Cl)cc2)cc(C(C)C)c1CC(=O)N=S(N)(=O)c1ccc(CN(C)C)cc1] |
| 169 | 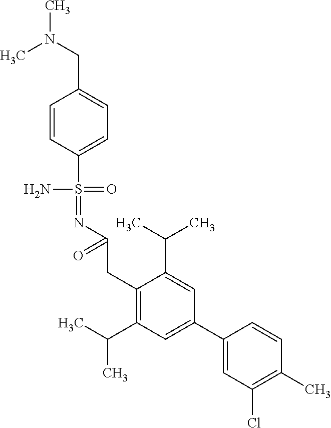 |
| 170 | 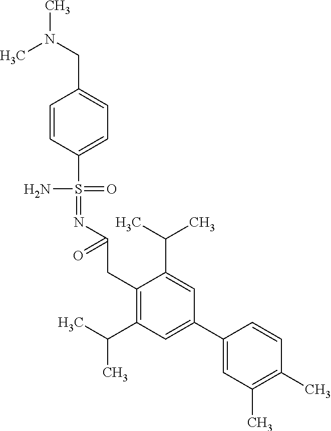 |
| 171 | 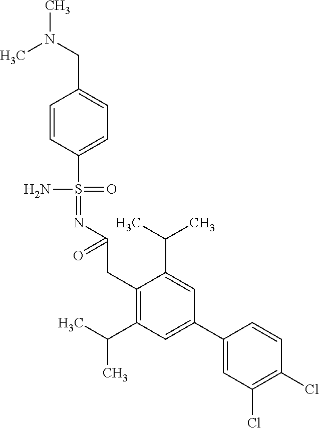 [SMILES: CC(C)c1cc(-c2ccc(Cl)c(Cl)c2)cc(C(C)C)c1CC(=O)N=S(N)(=O)c1ccc(CN(C)C)cc1] |
| 172 | 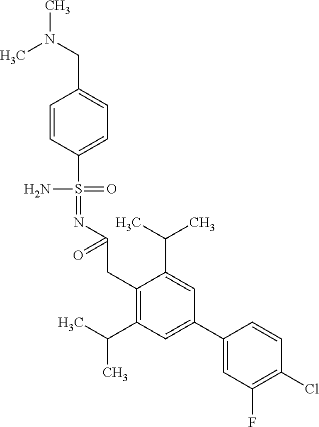 [SMILES: CC(C)c1cc(-c2ccc(Cl)c(F)c2)cc(C(C)C)c1CC(=O)N=S(N)(=O)c1ccc(CN(C)C)cc1] |
| 173 | 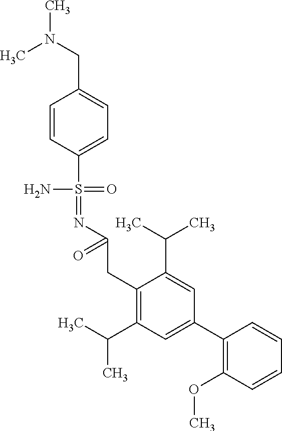 [SMILES: COc1ccccc1-c1cc(C(C)C)c(CC(=O)N=S(N)(=O)c2ccc(CN(C)C)cc2)c(C(C)C)c1] |
| 174 | 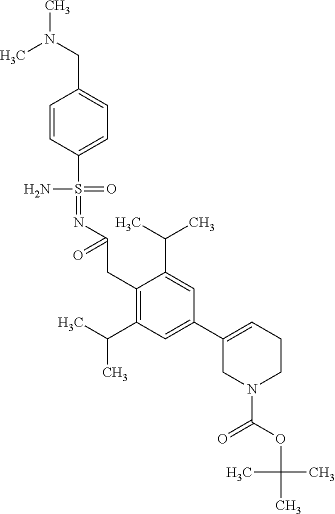 [SMILES: CC(C)c1cc(C2=CCCN(C(=O)OC(C)(C)C)C2)cc(C(C)C)c1CC(=O)N=S(N)(=O)c1ccc(CN(C)C)cc1] |
| 175 | 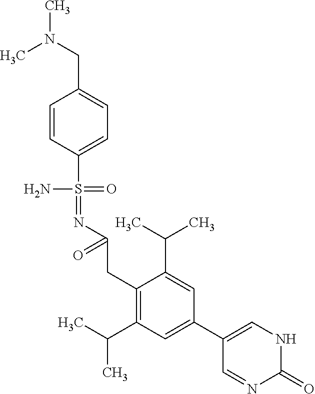 [SMILES: CC(C)c1cc(-c2cnc(=O)[nH]c2)cc(C(C)C)c1CC(=O)N=S(N)(=O)c1ccc(CN(C)C)cc1] |
| 176 | 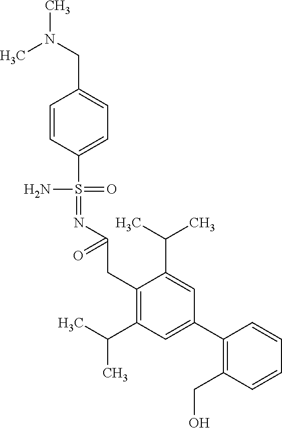 [SMILES: CC(C)c1cc(-c2ccccc2CO)cc(C(C)C)c1CC(=O)N=S(N)(=O)c1ccc(CN(C)C)cc1] |
| 177 | 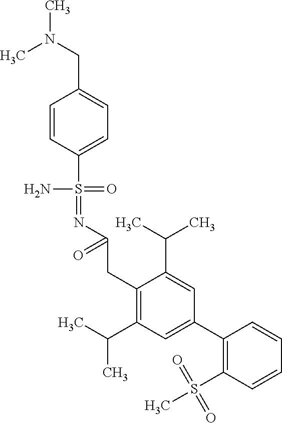 [SMILES: CC(C)c1cc(-c2ccccc2S(C)(=O)=O)cc(C(C)C)c1CC(=O)N=S(N)(=O)c1ccc(CN(C)C)cc1] |
| 178 | 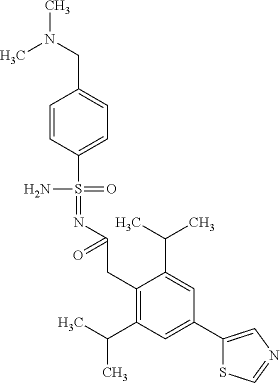 [SMILES: CC(C)c1cc(-c2cncs2)cc(C(C)C)c1CC(=O)N=S(N)(=O)c1ccc(CN(C)C)cc1] |
| 179 | 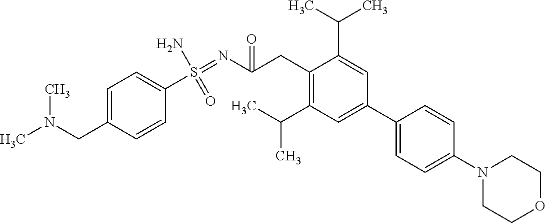 [SMILES: CC(C)c1cc(-c2ccc(N3CCOCC3)cc2)cc(C(C)C)c1CC(=O)N=S(N)(=O)c1ccc(CN(C)C)cc1] |
| 180 | 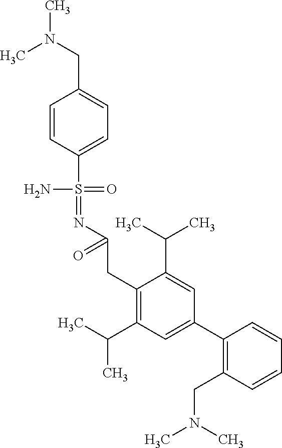 [SMILES: CC(C)c1cc(-c2ccccc2CN(C)C)cc(C(C)C)c1CC(=O)N=S(N)(=O)c1ccc(CN(C)C)cc1] |
| 181 | 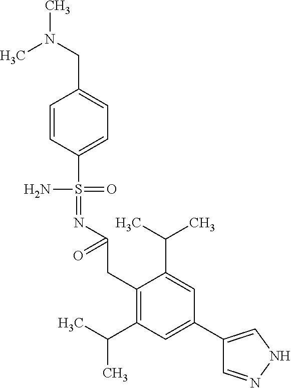 [SMILES: CC(C)c1cc(-c2cn[nH]c2)cc(C(C)C)c1CC(=O)N=S(N)(=O)c1ccc(CN(C)C)cc1] |
| 182 | 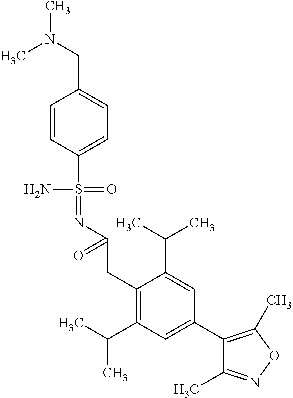 [SMILES: Cc1noc(C)c1-c1cc(C(C)C)c(CC(=O)N=S(N)(=O)c2ccc(CN(C)C)cc2)c(C(C)C)c1] |
| 183 | 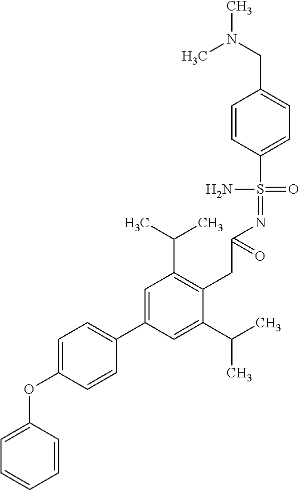 [SMILES: CC(C)c1cc(-c2ccc(Oc3ccccc3)cc2)cc(C(C)C)c1CC(=O)N=S(N)(=O)c1ccc(CN(C)C)cc1] |
| 184 | 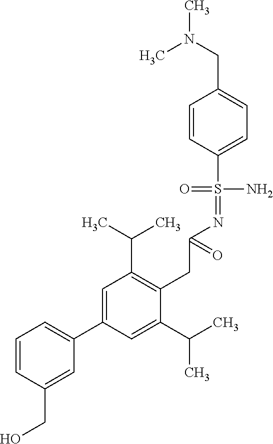 [SMILES: CC(C)c1cc(-c2cccc(CO)c2)cc(C(C)C)c1CC(=O)N=S(N)(=O)c1ccc(CN(C)C)cc1] |
| 185 | 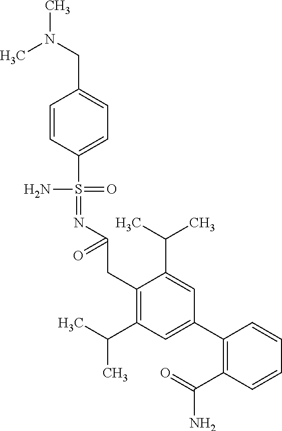 [SMILES: CC(C)c1cc(-c2ccccc2C(N)=O)cc(C(C)C)c1CC(=O)N=S(N)(=O)c1ccc(CN(C)C)cc1] |
| 186 | 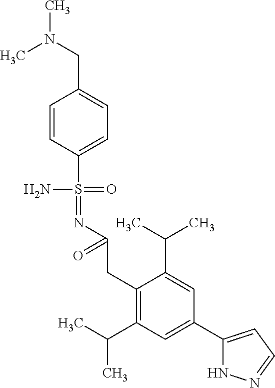 [SMILES: CC(C)c1cc(-c2ccn[nH]2)cc(C(C)C)c1CC(=O)N=S(N)(=O)c1ccc(CN(C)C)cc1] |
| 187 | 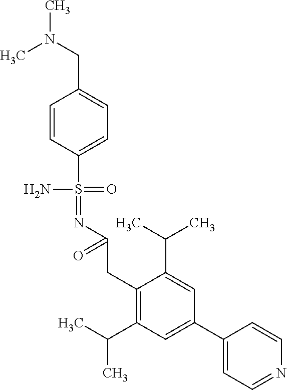 [SMILES: CC(C)c1cc(-c2ccncc2)cc(C(C)C)c1CC(=O)N=S(N)(=O)c1ccc(CN(C)C)cc1] |
| 188 | 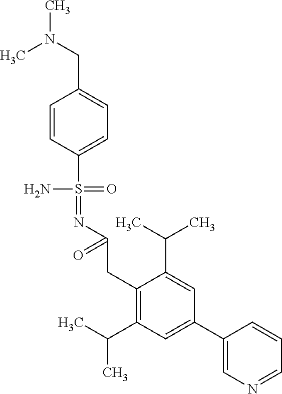 [SMILES: CC(C)c1cc(-c2cccnc2)cc(C(C)C)c1CC(=O)N=S(N)(=O)c1ccc(CN(C)C)cc1] |
| 189 | 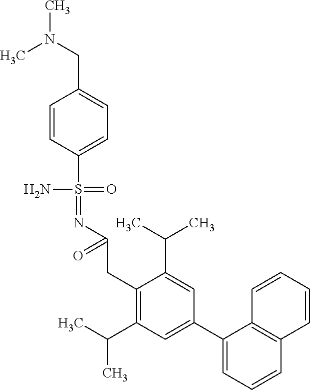 [SMILES: CC(C)c1cc(-c2cccc3ccccc23)cc(C(C)C)c1CC(=O)N=S(N)(=O)c1ccc(CN(C)C)cc1] |
| 190 | 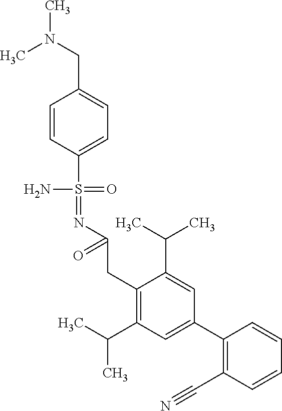 [SMILES: CC(C)c1cc(-c2ccccc2C#N)cc(C(C)C)c1CC(=O)N=S(N)(=O)c1ccc(CN(C)C)cc1] |
| 191 | 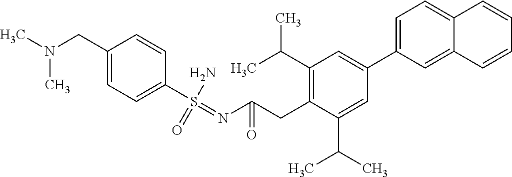 [SMILES: CC(C)c1cc(-c2ccc3ccccc3c2)cc(C(C)C)c1CC(=O)N=S(N)(=O)c1ccc(CN(C)C)cc1] |
| 192 | 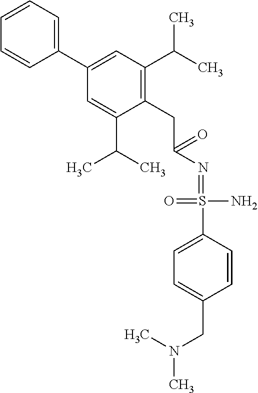 [SMILES: CC(C)c1cc(-c2ccccc2)cc(C(C)C)c1CC(=O)N=S(N)(=O)c1ccc(CN(C)C)cc1] |
| 193 | 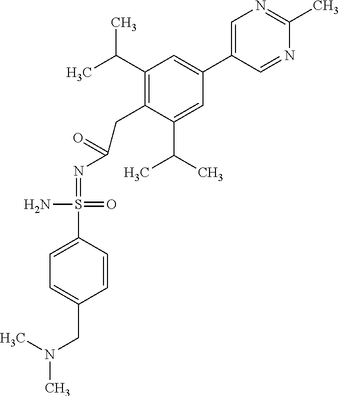 |
| 194 | 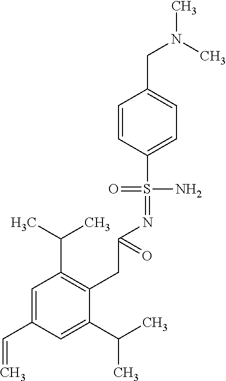 [SMILES: C=Cc1cc(C(C)C)c(CC(=O)N=S(N)(=O)c2ccc(CN(C)C)cc2)c(C(C)C)c1] |
| 195 | 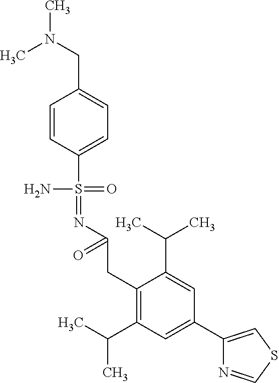 [SMILES: CC(C)c1cc(-c2cscn2)cc(C(C)C)c1CC(=O)N=S(N)(=O)c1ccc(CN(C)C)cc1] |
| 196 | 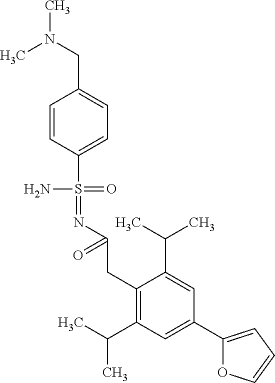 [SMILES: CC(C)c1cc(-c2ccco2)cc(C(C)C)c1CC(=O)N=S(N)(=O)c1ccc(CN(C)C)cc1] |
| 197 | 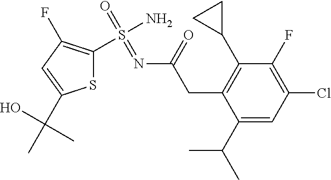 [SMILES: CC(C)c1cc(Cl)c(F)c(C2CC2)c1CC(=O)N=S(N)(=O)c1sc(C(C)(C)O)cc1F] |
| 197a | 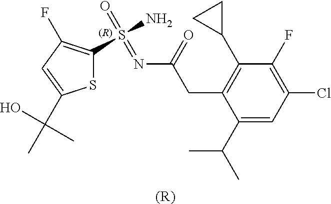 |
| 197b | 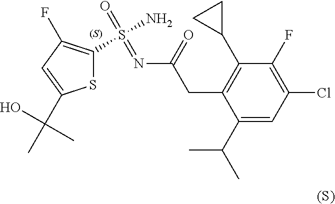 |
| 198 | 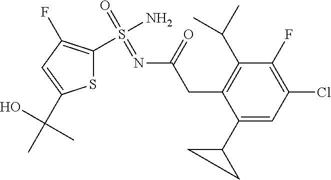 [SMILES: CC(C)c1c(F)c(Cl)cc(C2CC2)c1CC(=O)N=S(N)(=O)c1sc(C(C)(C)O)cc1F] |
| 198a | 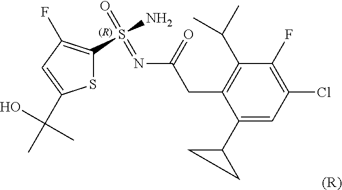 |
| 198b | 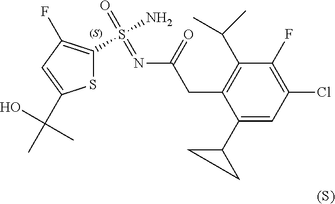 |

and pharmaceutically acceptable salts thereof.

In another aspect, an NLRP3 antagonist is a compound selected from the group consisting of the compounds in Table 16 below:

Table 16

| | |
|---|---|
| 201 | 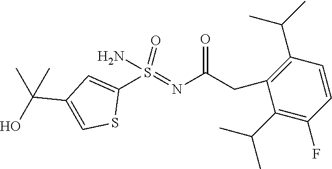 [SMILES: CC(C)c1ccc(F)c(C(C)C)c1CC(=O)N=S(N)(=O)c1cc(C(C)(C)O)cs1] |
| 201b | 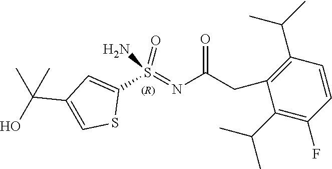 |
| 201a | 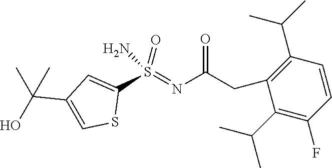 [SMILES: CC(C)c1ccc(F)c(C(C)C)c1CC(=O)N=[S@](N)(=O)c1cc(C(C)(C)O)cs1] |
| 202 | 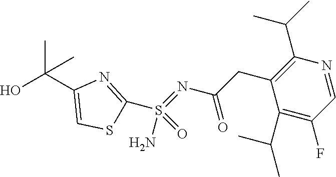 [SMILES: CC(C)c1ncc(F)c(C(C)C)c1CC(=O)N=S(N)(=O)c1nc(C(C)(C)O)cs1] |
| 202b | 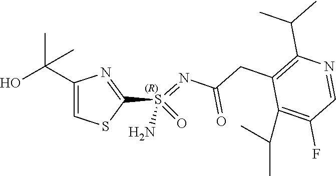 |
| 202a | 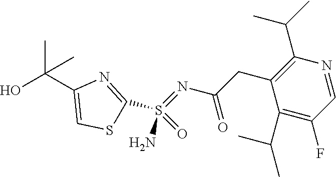 [SMILES: CC(C)c1ncc(F)c(C(C)C)c1CC(=O)N=[S@](N)(=O)c1nc(C(C)(C)O)cs1] |
| 203 | 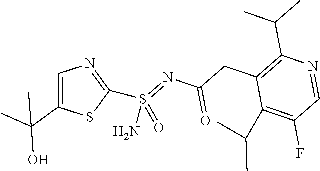 [SMILES: CC(C)c1ncc(F)c(C(C)C)c1CC(=O)N=S(N)(=O)c1ncc(C(C)(C)O)s1] |
| 203b | 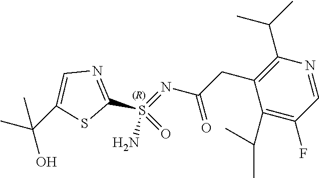 |
| 203a | 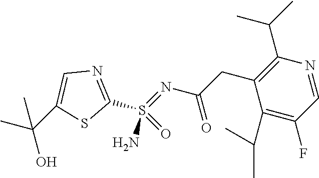 [SMILES: CC(C)c1ncc(F)c(C(C)C)c1CC(=O)N=[S@](N)(=O)c1ncc(C(C)(C)O)s1] |
| 204 | 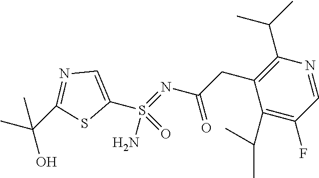 [SMILES: CC(C)c1ncc(F)c(C(C)C)c1CC(=O)N=S(N)(=O)c1cnc(C(C)(C)O)s1] |
| 204b | 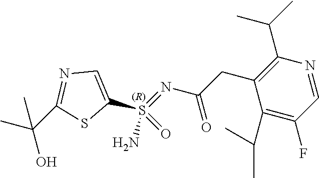 |
| 204a | 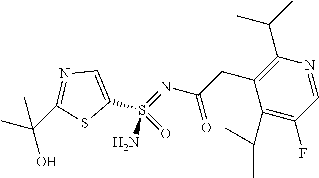 [SMILES: CC(C)c1ncc(F)c(C(C)C)c1CC(=O)N=[S@](N)(=O)c1cnc(C(C)(C)O)s1] |
| 205 | 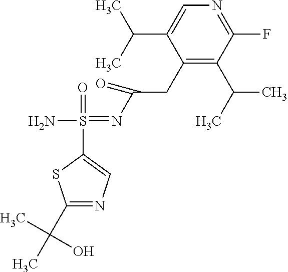 [SMILES: CC(C)c1cnc(F)c(C(C)C)c1CC(=O)N=S(N)(=O)c1cnc(C(C)(C)O)s1] |
| 206 | 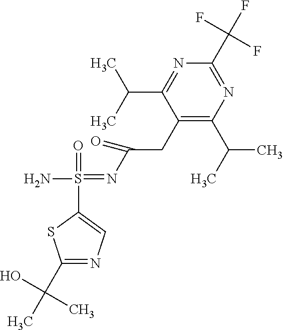 [SMILES: CC(C)c1nc(C(F)(F)F)nc(C(C)C)c1CC(=O)N=S(N)(=O)c1cnc(C(C)(C)O)s1] |
| 207 | 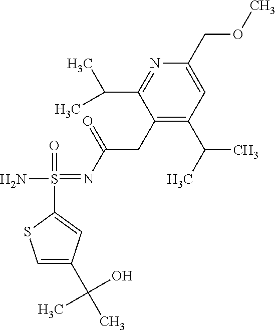 [SMILES: COCc1cc(C(C)C)c(CC(=O)N=S(N)(=O)c2cc(C(C)(C)O)cs2)c(C(C)C)n1] |
| 208 | 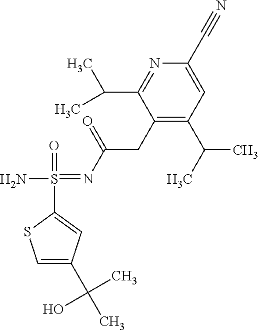 [SMILES: CC(C)c1cc(C#N)nc(C(C)C)c1CC(=O)N=S(N)(=O)c1cc(C(C)(C)O)cs1] |
| 209 | 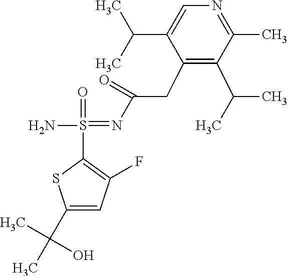 [SMILES: Cc1ncc(C(C)C)c(CC(=O)N=S(N)(=O)c2sc(C(C)(C)O)cc2F)c1C(C)C] |
| 210 | 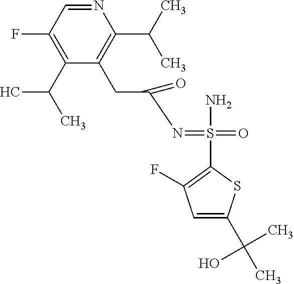 [SMILES: CC(C)c1ncc(F)c(C(C)C)c1CC(=O)N=S(N)(=O)c1sc(C(C)(C)O)cc1F] |
| 210a | 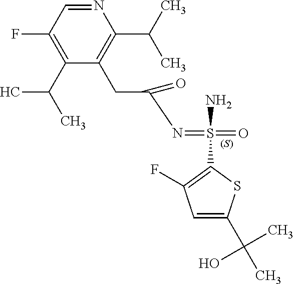 |
| 210b | 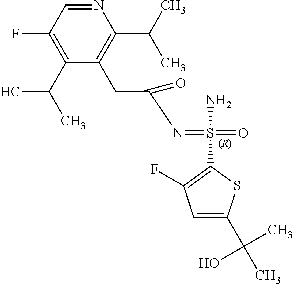 |
| 211 | 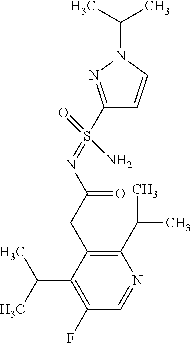 [SMILES: CC(C)c1ncc(F)c(C(C)C)c1CC(=O)N=S(N)(=O)c1ccn(C(C)C)n1] |
| 212 | 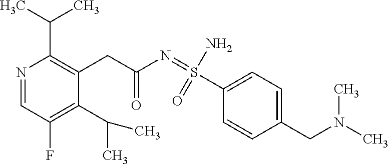 [SMILES: CC(C)c1ncc(F)c(C(C)C)c1CC(=O)N=S(N)(=O)c1ccc(CN(C)C)cc1] |
| 213 | 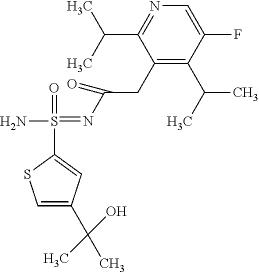 [SMILES: CC(C)c1ncc(F)c(C(C)C)c1CC(=O)N=S(N)(=O)c1cc(C(C)(C)O)cs1] |
| 213a | 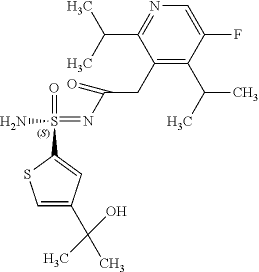 |
| 213b | 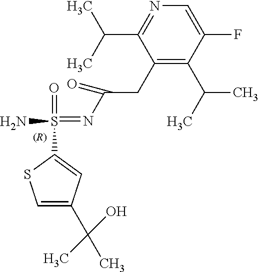 |
| 214 | 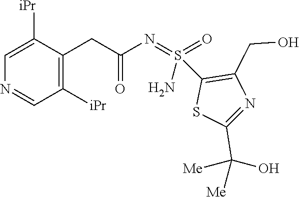 [SMILES: CC(C)c1cncc(C(C)C)c1CC(=O)N=S(N)(=O)c1sc(C(C)(C)O)nc1CO] |
| 215 | 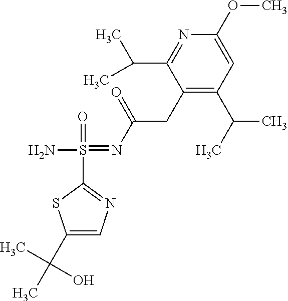 [SMILES: COc1cc(C(C)C)c(CC(=O)N=S(N)(=O)c2ncc(C(C)(C)O)s2)c(C(C)C)n1] |
| 216 | 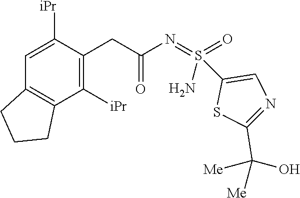 [SMILES: CC(C)c1cc2c(c(C(C)C)c1CC(=O)N=S(N)(=O)c1cnc(C(C)(C)O)s1)CCC2] |
| 216a | 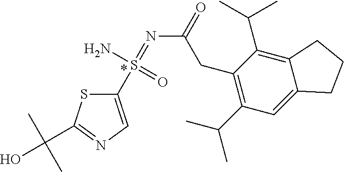 [SMILES: CC(C)c1cc2c(c(C(C)C)c1CC(=O)N=S(N)(=O)c1cnc(C(C)(C)O)s1)CCC2] |
| 216b | 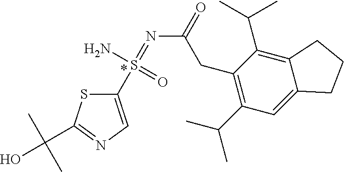 [SMILES: CC(C)c1cc2c(c(C(C)C)c1CC(=O)N=S(N)(=O)c1cnc(C(C)(C)O)s1)CCC2] |
| 217 | 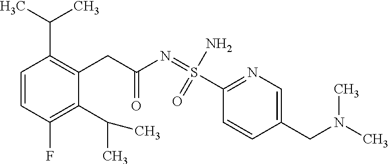 [SMILES: CC(C)c1ccc(F)c(C(C)C)c1CC(=O)N=S(N)(=O)c1ccc(CN(C)C)cn1] |
| 217a | 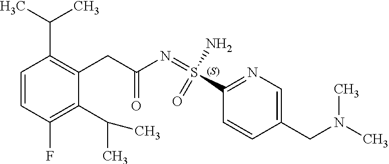 |
| 217b | 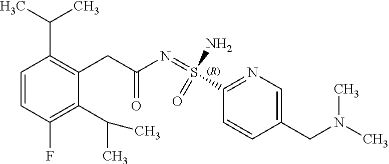 |
| 218 | 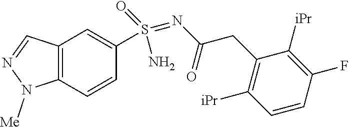 [SMILES: CC(C)c1ccc(F)c(C(C)C)c1CC(=O)N=S(N)(=O)c1ccc2c(cnn2C)c1] |
| 218a | 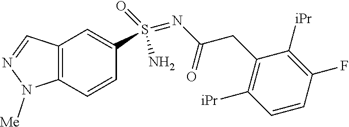 [SMILES: CC(C)c1ccc(F)c(C(C)C)c1CC(=O)N=[S@](N)(=O)c1ccc2c(cnn2C)c1] |
| 218b | 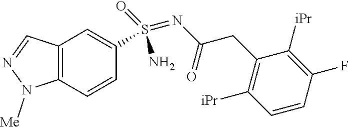 [SMILES: CC(C)c1ccc(F)c(C(C)C)c1CC(=O)N=[S@@](N)(=O)c1ccc2c(cnn2C)c1] |
| 219 | 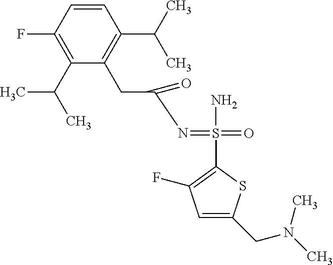 [SMILES: CC(C)c1ccc(F)c(C(C)C)c1CC(=O)N=S(N)(=O)c1sc(CN(C)C)cc1F] |
| 220 | 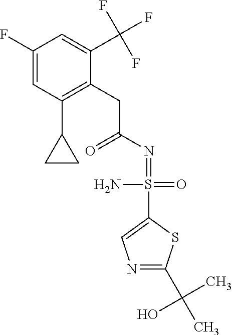 [SMILES: CC(C)(O)c1ncc(S(N)(=O)=NC(=O)Cc2c(C3CC3)cc(F)cc2C(F)(F)F)s1] |
| 220a | 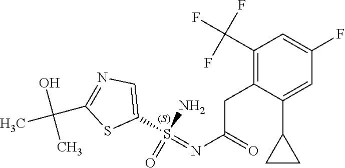 |
| 220b | 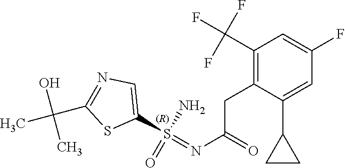 |
| 221 | 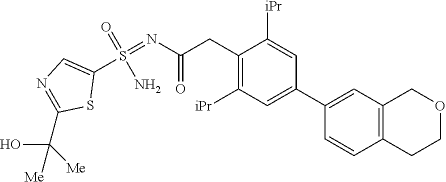 [SMILES: CC(C)c1cc(-c2ccc3c(c2)COCC3)cc(C(C)C)c1CC(=O)N=S(N)(=O)c1cnc(C(C)(C)O)s1] |
| 221a | 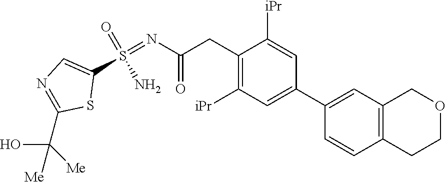 [SMILES: CC(C)c1cc(-c2ccc3c(c2)COCC3)cc(C(C)C)c1CC(=O)N=[S@](N)(=O)c1cnc(C(C)(C)O)s1] |
| 221b | 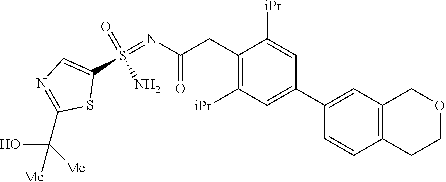 [SMILES: CC(C)c1cc(-c2ccc3c(c2)COCC3)cc(C(C)C)c1CC(=O)N=[S@](N)(=O)c1cnc(C(C)(C)O)s1] |
| 222 | 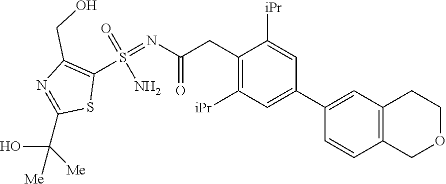 [SMILES: CC(C)c1cc(-c2ccc3c(c2)CCOC3)cc(C(C)C)c1CC(=O)N=S(N)(=O)c1sc(C(C)(C)O)nc1CO] |
| 224 | 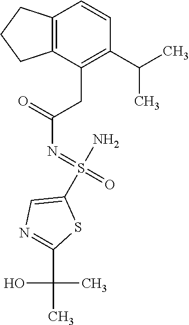 [SMILES: CC(C)c1ccc2c(c1CC(=O)N=S(N)(=O)c1cnc(C(C)(C)O)s1)CCC2] |
| 224b | 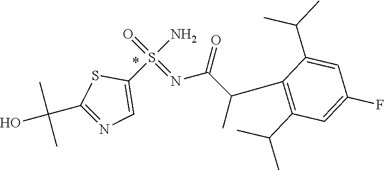 [SMILES: CC(C)c1cc(F)cc(C(C)C)c1C(C)C(=O)N=S(N)(=O)c1cnc(C(C)(C)O)s1] |
| 224ba | 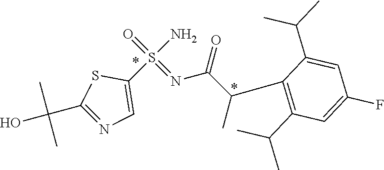 [SMILES: CC(C)c1cc(F)cc(C(C)C)c1C(C)C(=O)N=S(N)(=O)c1cnc(C(C)(C)O)s1] |
| 224aa | 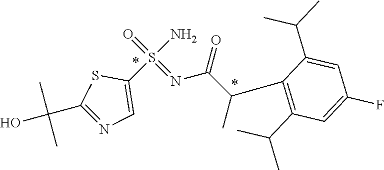 [SMILES: CC(C)c1cc(F)cc(C(C)C)c1C(C)C(=O)N=S(N)(=O)c1cnc(C(C)(C)O)s1] |
| 225 | 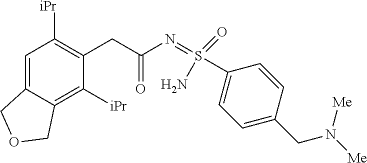 [SMILES: CC(C)c1cc2c(c(C(C)C)c1CC(=O)N=S(N)(=O)c1ccc(CN(C)C)cc1)COC2] |
| 225a | 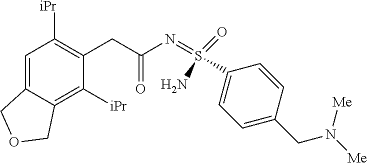 [SMILES: CC(C)c1cc2c(c(C(C)C)c1CC(=O)N=[S@](N)(=O)c1ccc(CN(C)C)cc1)COC2] |
| 225b | 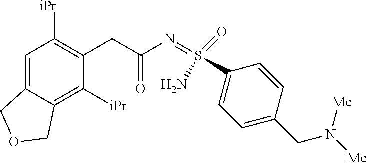 [SMILES: CC(C)c1cc2c(c(C(C)C)c1CC(=O)N=[S@@](N)(=O)c1ccc(CN(C)C)cc1)COC2] |
| 226 | 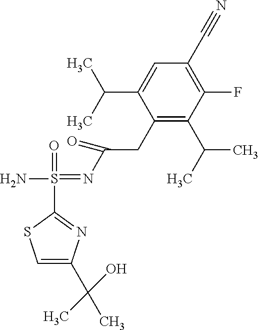 [SMILES: CC(C)c1cc(C#N)c(F)c(C(C)C)c1CC(=O)N=S(N)(=O)c1nc(C(C)(C)O)cs1] |
| 226a | 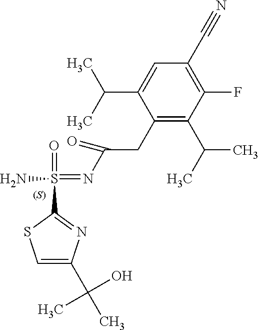 |
| 226b | 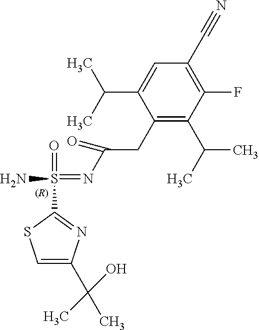 |
| 227 | 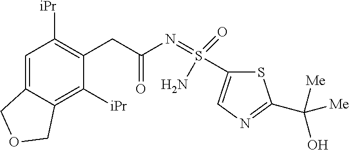 [SMILES: CC(C)c1cc2c(c(C(C)C)c1CC(=O)N=S(N)(=O)c1cnc(C(C)(C)O)s1)COC2] |
| 227a | 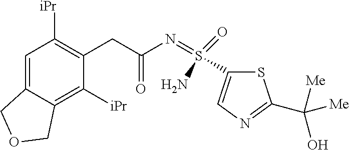 [SMILES: CC(C)c1cc2c(c(C(C)C)c1CC(=O)N=[S@](N)(=O)c1cnc(C(C)(C)O)s1)COC2] |
| 227b | 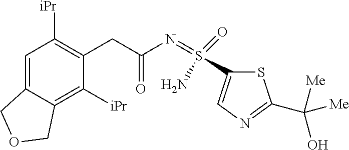 [SMILES: CC(C)c1cc2c(c(C(C)C)c1CC(=O)N=[S@@](N)(=O)c1cnc(C(C)(C)O)s1)COC2] |
| 228 | 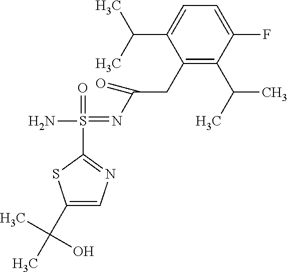 [SMILES: CC(C)c1ccc(F)c(C(C)C)c1CC(=O)N=S(N)(=O)c1ncc(C(C)(C)O)s1] |
| 228a | 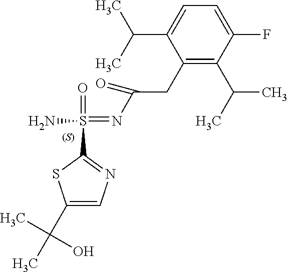 |
| 228b | 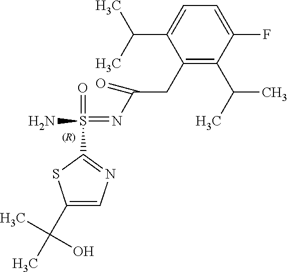 |
| 229 | 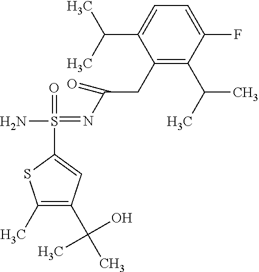 [SMILES: Cc1sc(S(N)(=O)=NC(=O)Cc2c(C(C)C)ccc(F)c2C(C)C)cc1C(C)(C)O] |
| 229a | 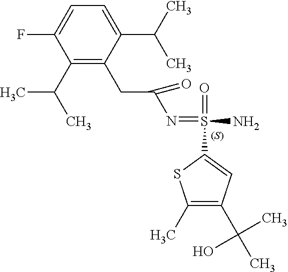 |
| 229b | 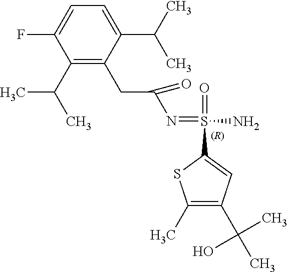 |
| 230 | 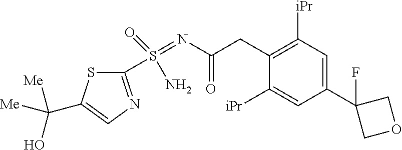 [SMILES: CC(C)c1cc(C2(F)COC2)cc(C(C)C)c1CC(=O)N=S(N)(=O)c1ncc(C(C)(C)O)s1] |
| 231 | 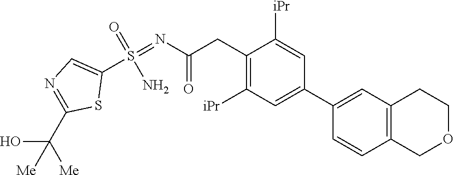 [SMILES: CC(C)c1cc(-c2ccc3c(c2)CCOC3)cc(C(C)C)c1CC(=O)N=S(N)(=O)c1cnc(C(C)(C)O)s1] |
| 232 | 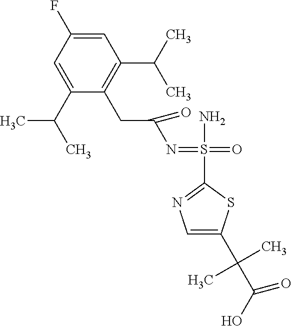 [SMILES: CC(C)c1cc(F)cc(C(C)C)c1CC(=O)N=S(N)(=O)c1ncc(C(C)(C)C(=O)O)s1] |
| 233 | 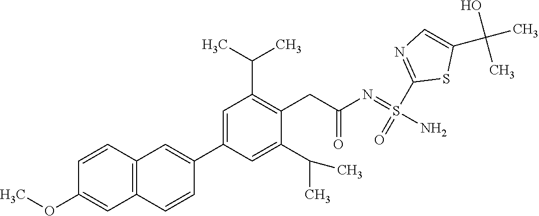 [SMILES: COc1ccc2cc(-c3cc(C(C)C)c(CC(=O)N=S(N)(=O)c4ncc(C(C)(C)O)s4)c(C(C)C)c3)ccc2c1] |
| 234 | 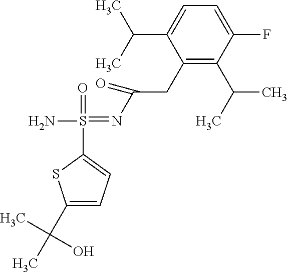 [SMILES: CC(C)c1ccc(F)c(C(C)C)c1CC(=O)N=S(N)(=O)c1ccc(C(C)(C)O)s1] |
| 234a | 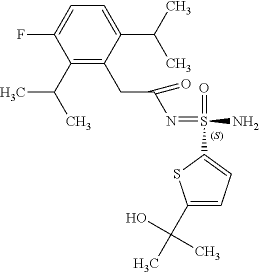 |
| 234b | 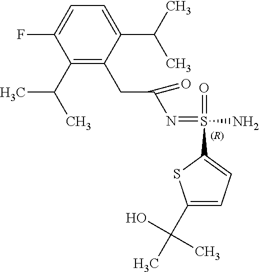 |
| 235 | 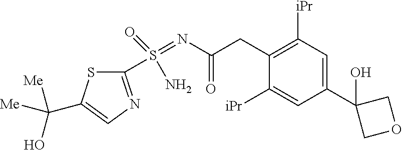 [SMILES: CC(C)c1cc(C2(O)COC2)cc(C(C)C)c1CC(=O)N=S(N)(=O)c1ncc(C(C)(C)O)s1] |
| 236 | 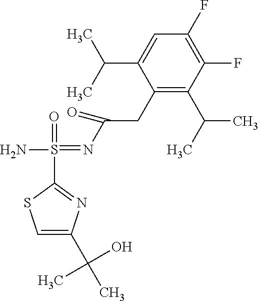 [SMILES: CC(C)c1cc(F)c(F)c(C(C)C)c1CC(=O)N=S(N)(=O)c1nc(C(C)(C)O)cs1] |
| 236a | 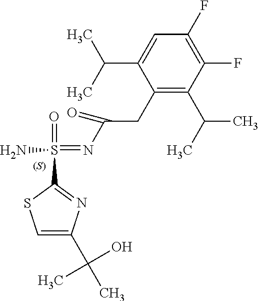 |
| 236b | 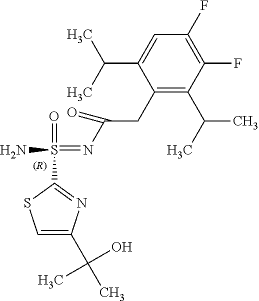 |
| 237 | 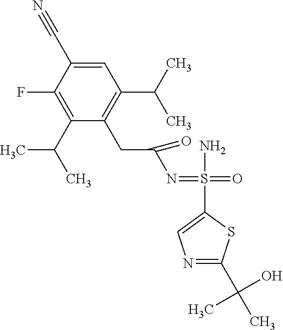 [SMILES: CC(C)c1cc(C#N)c(F)c(C(C)C)c1CC(=O)N=S(N)(=O)c1cnc(C(C)(C)O)s1] |
| 238 | 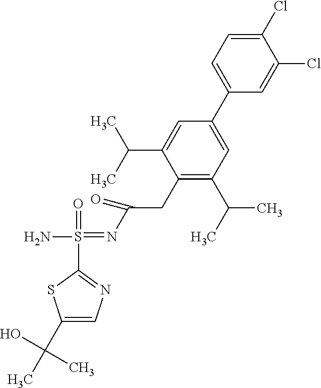 [SMILES: CC(C)c1cc(-c2ccc(Cl)c(Cl)c2)cc(C(C)C)c1CC(=O)N=S(N)(=O)c1ncc(C(C)(C)O)s1] |
| 238a | 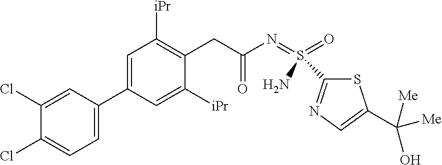 [SMILES: CC(C)c1cc(-c2ccc(Cl)c(Cl)c2)cc(C(C)C)c1CC(=O)N=[S@](N)(=O)c1ncc(C(C)(C)O)s1] |
| 238b | 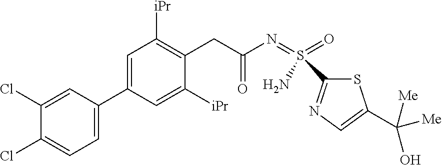 [SMILES: CC(C)c1cc(-c2ccc(Cl)c(Cl)c2)cc(C(C)C)c1CC(=O)N=[S@@](N)(=O)c1ncc(C(C)(C)O)s1] |
| 239 | 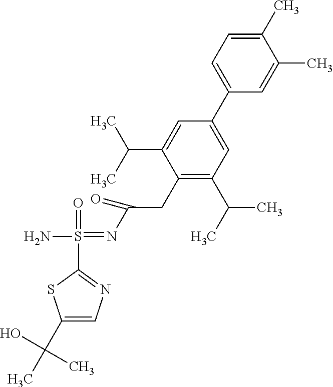 [SMILES: Cc1ccc(-c2cc(C(C)C)c(CC(=O)N=S(N)(=O)c3ncc(C(C)(C)O)s3)c(C(C)C)c2)cc1C] |
| 239a | 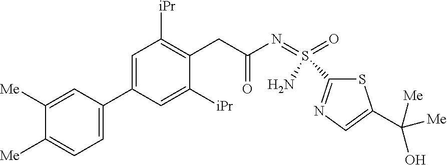 [SMILES: Cc1ccc(-c2cc(C(C)C)c(CC(=O)N=[S@@](N)(=O)c3ncc(C(C)(C)O)s3)c(C(C)C)c2)cc1C] |
| 239b | 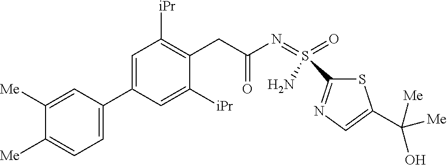 [SMILES: Cc1ccc(-c2cc(C(C)C)c(CC(=O)N=[S@@](N)(=O)c3ncc(C(C)(C)O)s3)c(C(C)C)c2)cc1C] |
| 240 | 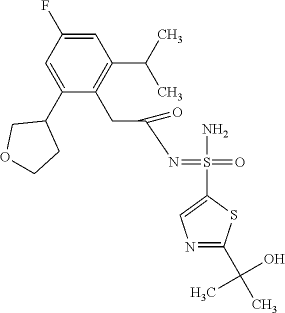 [SMILES: CC(C)c1cc(F)cc(C2CCOC2)c1CC(=O)N=S(N)(=O)c1cnc(C(C)(C)O)s1] |
| 241 | 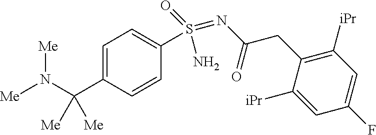 [SMILES: CC(C)c1cc(F)cc(C(C)C)c1CC(=O)N=S(N)(=O)c1ccc(C(C)(C)N(C)C)cc1] |
| 242 | 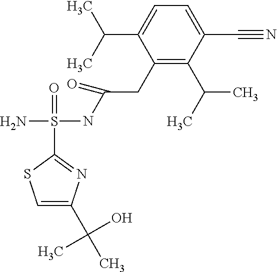 |
| 242a | 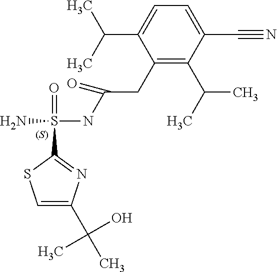 |
| 242b | 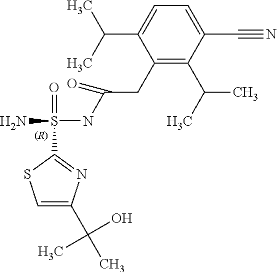 |
| 243 | 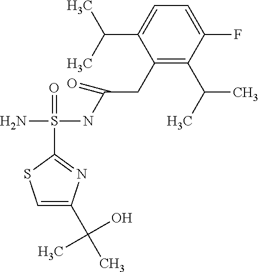 |
| 243a | 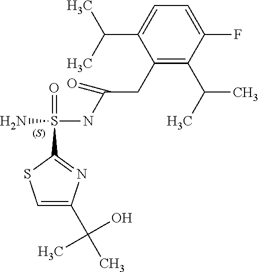 |
| 243b | 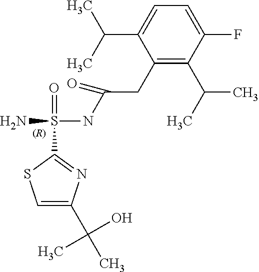 |
| 244 | 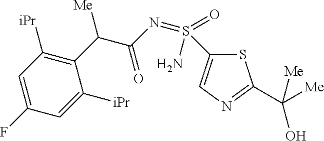 [SMILES: CC(C)c1cc(F)cc(C(C)C)c1C(C)C(=O)N=S(N)(=O)c1cnc(C(C)(C)O)s1] |
| 244aa | 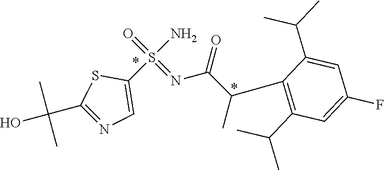 [SMILES: CC(C)c1cc(F)cc(C(C)C)c1C(C)C(=O)N=S(N)(=O)c1cnc(C(C)(C)O)s1] |
| 244b | 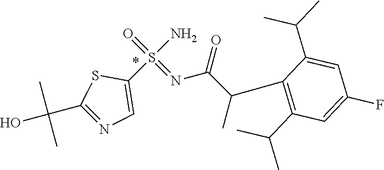 [SMILES: CC(C)c1cc(F)cc(C(C)C)c1C(C)C(=O)N=S(N)(=O)c1cnc(C(C)(C)O)s1] |
| 244ba | 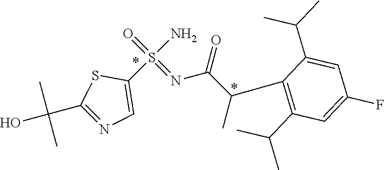 [SMILES: CC(C)c1cc(F)cc(C(C)C)c1C(C)C(=O)N=S(N)(=O)c1cnc(C(C)(C)O)s1] |
| 245 | 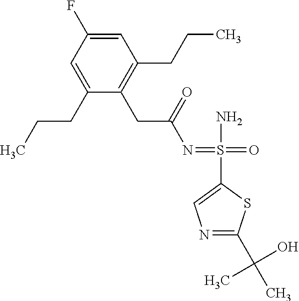 [SMILES: CCCc1cc(F)cc(CCC)c1CC(=O)N=S(N)(=O)c1cnc(C(C)(C)O)s1] |
| 246 | 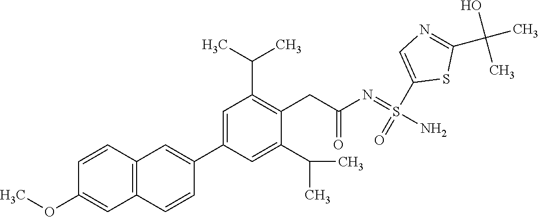 [SMILES: COc1ccc2cc(-c3cc(C(C)C)c(CC(=O)N=S(N)(=O)c4cnc(C(C)(C)O)s4)c(C(C)C)c3)ccc2c1] |
| 247 | 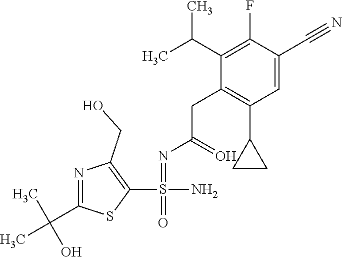 [SMILES: CC(C)c1c(F)c(C#N)cc(C2CC2)c1CC(=O)N=S(N)(=O)c1sc(C(C)(C)O)nc1CO] |
| 248 | 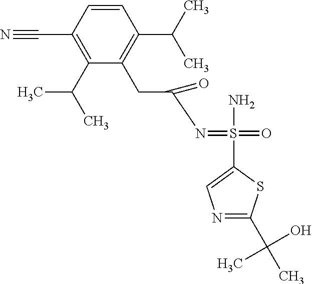 [SMILES: CC(C)c1ccc(C#N)c(C(C)C)c1CC(=O)N=S(N)(=O)c1cnc(C(C)(C)O)s1] |
| 249 | 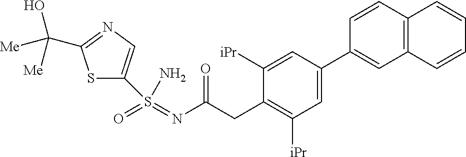 [SMILES: CC(C)c1cc(-c2ccc3ccccc3c2)cc(C(C)C)c1CC(=O)N=S(N)(=O)c1cnc(C(C)(C)O)s1] |
| 249a | 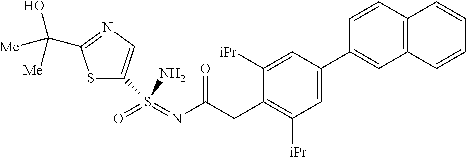 [SMILES: CC(C)c1cc(-c2ccc3ccccc3c2)cc(C(C)C)c1CC(=O)N=[S@](N)(=O)c1cnc(C(C)(C)O)s1] |
| 249b | 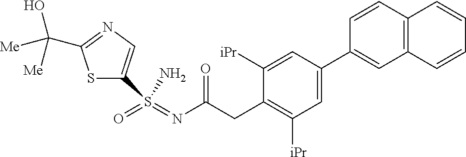 [SMILES: CC(C)c1cc(-c2ccc3ccccc3c2)cc(C(C)C)c1CC(=O)N=[S@@](N)(=O)c1cnc(C(C)(C)O)s1] |
| 250 | 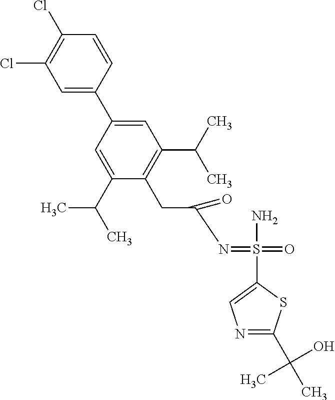 [SMILES: CC(C)c1cc(-c2ccc(Cl)c(Cl)c2)cc(C(C)C)c1CC(=O)N=S(N)(=O)c1cnc(C(C)(C)O)s1] |
| 251 | 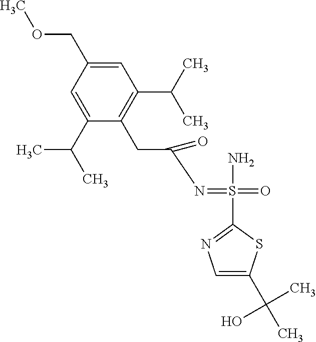 [SMILES: COCc1cc(C(C)C)c(CC(=O)N=S(N)(=O)c2ncc(C(C)(C)O)s2)c(C(C)C)c1] |
| 251a | 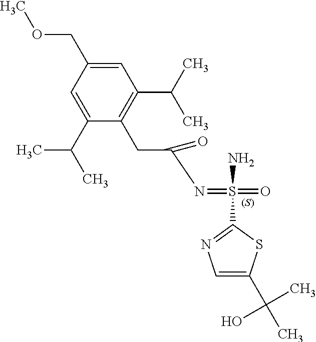 |
| 251b | 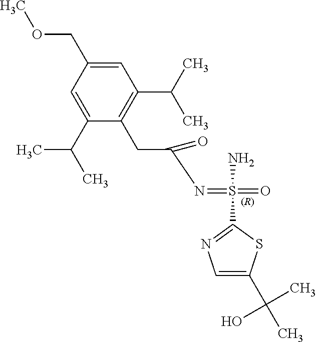 |
| 252 | 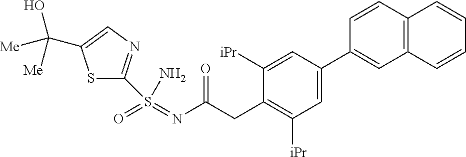 [SMILES: CC(C)c1cc(-c2ccc3ccccc3c2)cc(C(C)C)c1CC(=O)N=S(N)(=O)c1ncc(C(C)(C)O)s1] |
| 253 | 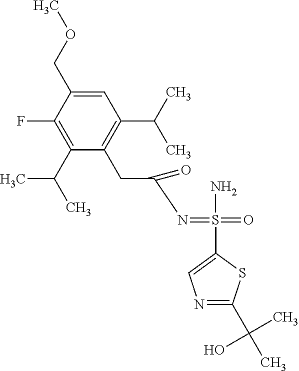 [SMILES: COCc1cc(C(C)C)c(CC(=O)N=S(N)(=O)c2cnc(C(C)(C)O)s2)c(C(C)C)c1F] |
| 254 | 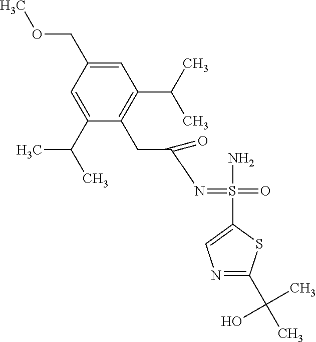 [SMILES: COCc1cc(C(C)C)c(CC(=O)N=S(N)(=O)c2cnc(C(C)(C)O)s2)c(C(C)C)c1] |
| 255 | 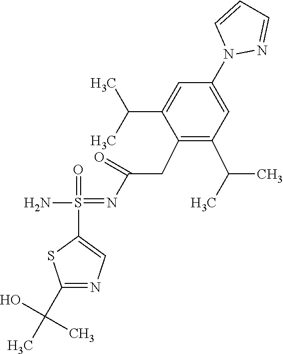 [SMILES: CC(C)c1cc(-n2cccn2)cc(C(C)C)c1CC(=O)N=S(N)(=O)c1cnc(C(C)(C)O)s1] |
| 256 | 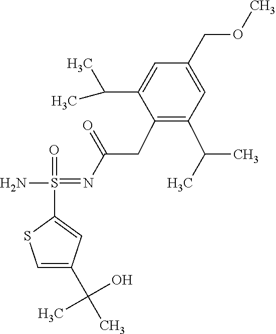 [SMILES: COCc1cc(C(C)C)c(CC(=O)N=S(N)(=O)c2cc(C(C)(C)O)cs2)c(C(C)C)c1] |
| 256a | 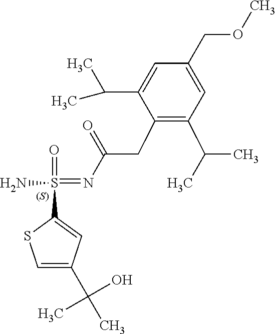 |
| 256b | 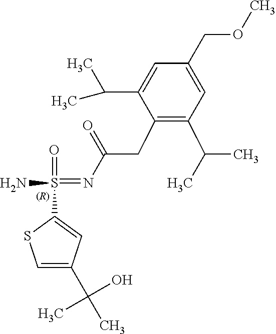 |
| 257 | 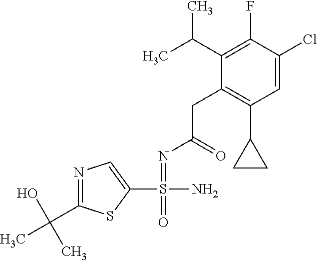 [SMILES: CC(C)c1c(F)c(Cl)cc(C2CC2)c1CC(=O)N=S(N)(=O)c1cnc(C(C)(C)O)s1] |
| 258 | 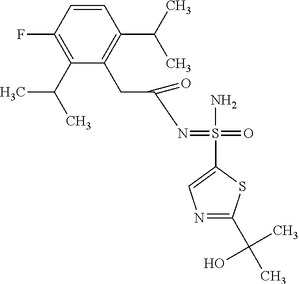 [SMILES: CC(C)c1ccc(F)c(C(C)C)c1CC(=O)N=S(N)(=O)c1cnc(C(C)(C)O)s1] |
| 258a | 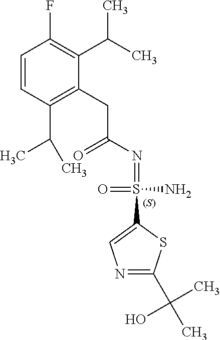 |
| 258b | 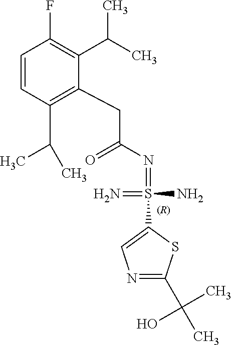 |
| 259 | 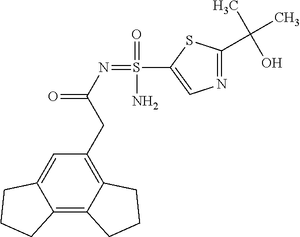 [SMILES: CC(C)(O)c1ncc(S(N)(=O)=NC(=O)Cc2cc3c(c4c2CCC4)CCC3)s1] |
| 259a | 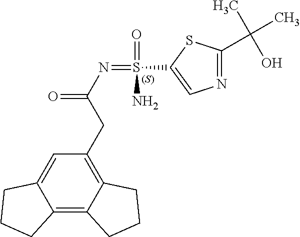 |
| 259b | 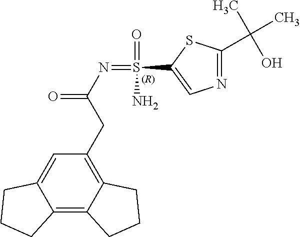 |
| 260 | 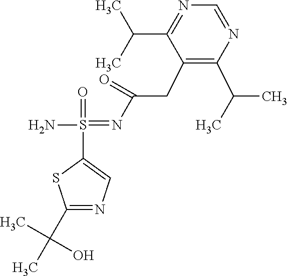 [SMILES: CC(C)c1ncnc(C(C)C)c1CC(=O)N=S(N)(=O)c1cnc(C(C)(C)O)s1] |
| 261a | 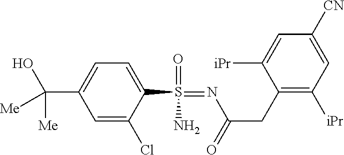 [SMILES: CC(C)c1cc(C#N)cc(C(C)C)c1CC(=O)N=[S@](N)(=O)c1ccc(C(C)(C)O)cc1Cl] |
| 261b | 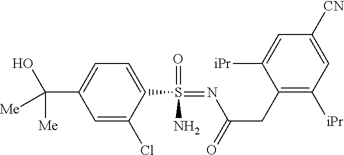 [SMILES: CC(C)c1cc(C#N)cc(C(C)C)c1CC(=O)N=[S@@](N)(=O)c1ccc(C(C)(C)O)cc1Cl] |
| 262a | 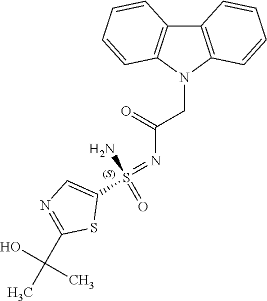 |
| 262b | 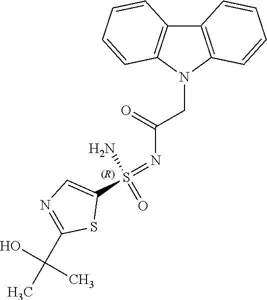 |
| 263 | 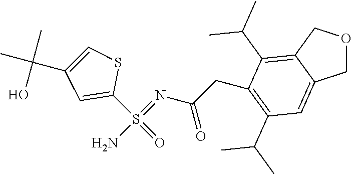 [SMILES: CC(C)c1cc2c(c(C(C)C)c1CC(=O)N=S(N)(=O)c1cc(C(C)(C)O)cs1)COC2] |
| 263a | 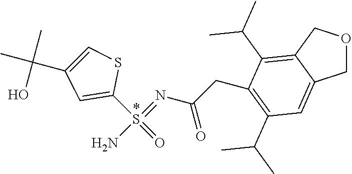 [SMILES: CC(C)c1cc2c(c(C(C)C)c1CC(=O)N=S(N)(=O)c1cc(C(C)(C)O)cs1)COC2] |
| 263b | 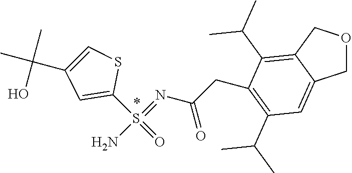 [SMILES: CC(C)c1cc2c(c(C(C)C)c1CC(=O)N=S(N)(=O)c1cc(C(C)(C)O)cs1)COC2] |
| 264 | 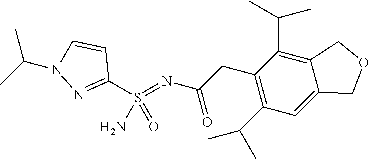 [SMILES: CC(C)c1cc2c(c(C(C)C)c1CC(=O)N=S(N)(=O)c1ccn(C(C)C)n1)COC2] |
| 264a | 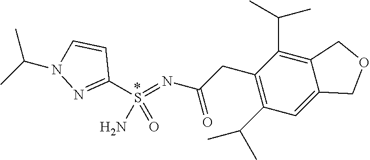 [SMILES: CC(C)c1cc2c(c(C(C)C)c1CC(=O)N=S(N)(=O)c1ccn(C(C)C)n1)COC2] |
| 264b | 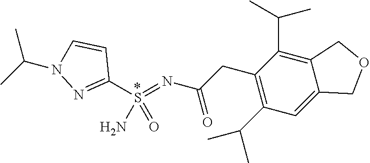 [SMILES: CC(C)c1cc2c(c(C(C)C)c1CC(=O)N=S(N)(=O)c1ccn(C(C)C)n1)COC2] |

and pharmaceutically acceptable salts thereof.

In another aspect, an NLRP3 antagonist is a compound selected from the group consisting of the compounds in Table 17 below:

TABLE 17

| | |
|---|---|
| 301 | 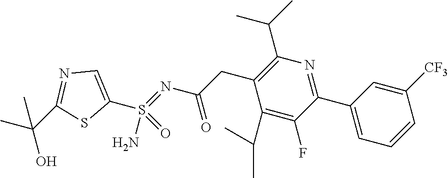 [SMILES: CC(C)c1nc(-c2cccc(C(F)(F)F)c2)c(F)c(C(C)C)c1CC(=O)N=S(N)(=O)c1cnc(C(C)(C)O)s1] |
| 302 | 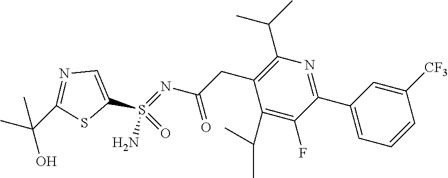 [SMILES: CC(C)c1nc(-c2cccc(C(F)(F)F)c2)c(F)c(C(C)C)c1CC(=O)N=[S@@](N)(=O)c1cnc(C(C)(C)O)s1] |
| 303 | 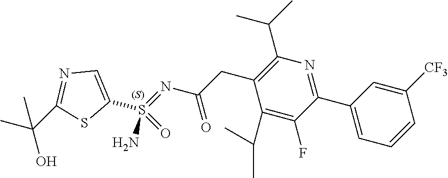 |
| 304 | 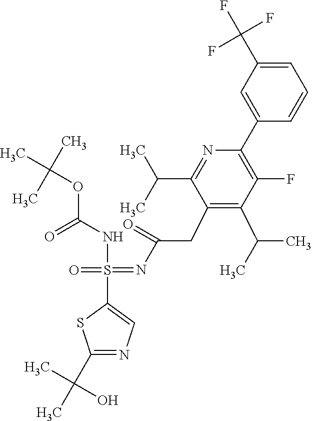 [SMILES: CC(C)c1nc(-c2cccc(C(F)(F)F)c2)c(F)c(C(C)C)c1CC(=O)N=S(=O)(NC(=O)OC(C)(C)C)c1cnc(C(C)(C)O)s1] |
| 305 | 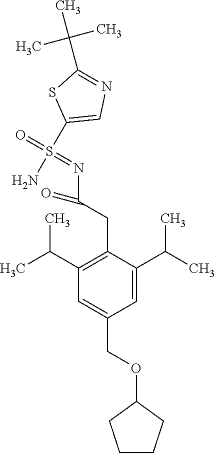 [SMILES: CC(C)c1cc(COC2CCCC2)cc(C(C)C)c1CC(=O)N=S(N)(=O)c1cnc(C(C)(C)C)s1] |
| 306 | 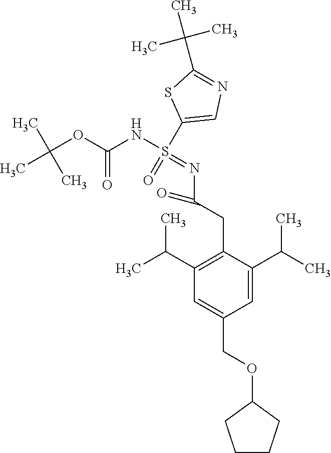 [SMILES: CC(C)c1cc(COC2CCCC2)cc(C(C)C)c1CC(=O)N=S(=O)(NC(=O)OC(C)(C)C)c1cnc(C(C)(C)C)s1] |
| 307 | 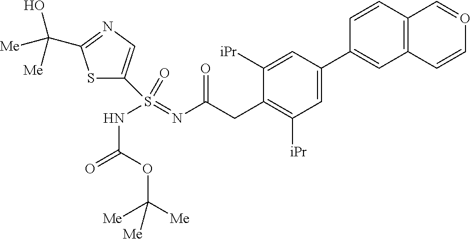 |
| 309 | 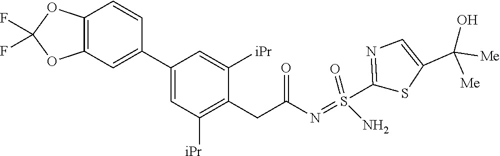 [SMILES: CC(C)c1cc(-c2ccc3c(c2)OC(F)(F)O3)cc(C(C)C)c1CC(=O)N=S(N)(=O)c1ncc(C(C)(C)O)s1] |
| 309a | 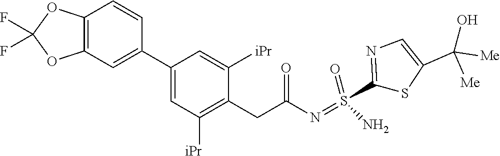 [SMILES: CC(C)c1cc(-c2ccc3c(c2)OC(F)(F)O3)cc(C(C)C)c1CC(=O)N=[S@@](N)(=O)c1ncc(C(C)(C)O)s1] |
| 309b | 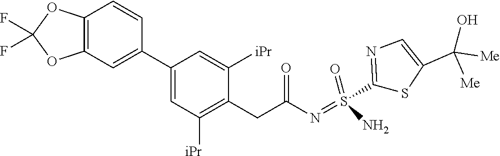 [SMILES: CC(C)c1cc(-c2ccc3c(c2)OC(F)(F)O3)cc(C(C)C)c1CC(=O)N=[S@](N)(=O)c1ncc(C(C)(C)O)s1] |
| 310 | 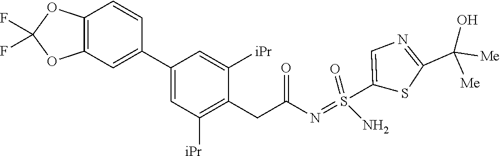 [SMILES: CC(C)c1cc(-c2ccc3c(c2)OC(F)(F)O3)cc(C(C)C)c1CC(=O)N=S(N)(=O)c1cnc(C(C)(C)O)s1] |

and pharmaceutically acceptable salts thereof.

In some embodiments of the compound of Formula XII, the sulfur in the moiety S(═O)(NHR³)═N— has (S) stereochemistry.

In some embodiments of the methods described herein, the compounds of Formula VII, VIII, XII and compounds listed in Tables 7, 8, and 15-18 above are administered in combination with an anti-TNFα agent; preferably in combination with Infliximab, Etanercept, Certolizumab pegol, Golimumab or Adalimumab; more preferably in combination with Adalimumab; more preferably in combination with Adalimumab.

In some embodiments of the compound of Formula XII, the sulfur in the moiety S(═O)(NHR³)═N— has (R) stereochemistry.

Compounds having Formula XII, as well as methods of making and using the same, are further described in PCT Application PCT/US2018/043330, entitled “Compounds And Compositions For Treating Conditions Associated With NLRP Activity”, filed on Jul. 23, 2018, which is incorporated herein by reference in its entirety.

In some of the embodiments described herein, the NLRP3 antagonist is selected from Table 18 below:

TABLE 18

| Compound Name | Structure | Reference |
|---|---|---|
| Parthenolide | 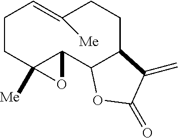 | Juliana, C. et al. J. Biol. Chem. 285,9792-9802 (2010), which is incorporated herein by reference in its entirety. |
| BAY-11-7082 | 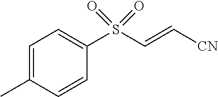 | Juliana, C. et al. J. Biol. Chem. 285,9792-9802 (2010), which is incorporated herein by reference in its entirety. |
| Glyburide | 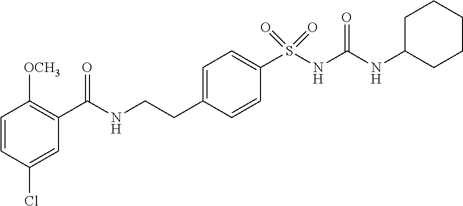 [SMILES: COc1ccc(Cl)cc1C(=O)NCCc1ccc(S(=O)(=O)NC(=O)NC2CCCCC2)cc1] | Lamkanfi, M. et al. J. Cell Biol. 187,61-70 (2009), which is incorporated herein by reference in its entirety. |
| 16673-34-0 | 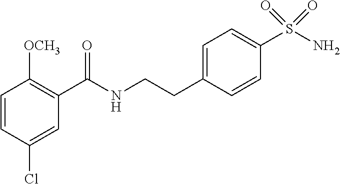 [SMILES: COc1ccc(Cl)cc1C(=O)NCCc1ccc(S(N)(=O)=O)cc1] | Marchetti, C. et al. J. Cardiovase. Pharmacol. 63, 316-322 (2014), which is incorporated herein by reference in its enfirety |
| CY-09 | 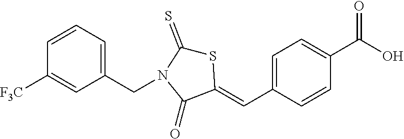 [SMILES: O=C(O)c1ccc(/C=C2\SC(=S)N(Cc3cccc(C(F)(F)F)c3)C2=O)cc1] | Jiang, H. et al. of. Exp. Med 214, 3219-3238 (2017), which is incorporated herein by reference in its entirety. |
| CP-456,773 | 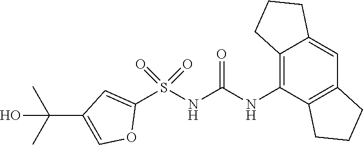 [SMILES: CC(C)(O)c1coc(S(=O)(=O)NC(=O)Nc2c3c(cc4c2CCC4)CCC3)c1] | Coll, R. C. et al. Nat. Med. 21, 248-255 (2015), which is incorporated herein by reference in its entirety |
| 3,4-methylenedioxy-β- nitrostyrene (MNS) | 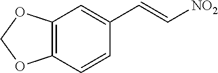 [SMILES: O=[N+]([O-])/C=C/c1ccc2c(c1)OCO2] | He, Y. et al. J. Biol. Chem. 289, 1142-1150 (2014), which is incorporated herein by reference in its entirety |
| Fenamate | 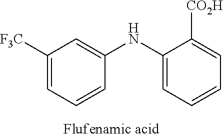 [SMILES: O=C(O)c1ccccc1Nc1cccc(C(F)(F)F)c1] | Daniels, M. J. D. et al. Nat. Commun. 7, 1-10 (2016), which is incorporated herein by reference in its entirety |
| 5-nitro-2-(3- phenylpropylamino) benzoic acid (NPPD) | | Compan, V. et al. Immunity 37, 487-500 (2012), which is incorporated herein by reference in its entirety. |
| OLT1177 | 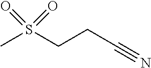 | Marchetti, C. et al. Proc. Natl Acad. Sci. 115, E1530- E1539 (2018), ).which is incorporated herein by reference in its entirety. |
| Fclla-2 | 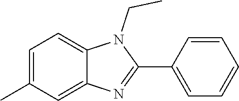 [SMILES: CCn1c(-c2ccccc2)nc2cc(C)ccc21] | Liu, W. et al. Biochem. Pharmacol. 85, 1504-1512 (2013), which is incorporated herein by reference in its entirety. |
| Spirodalesol | 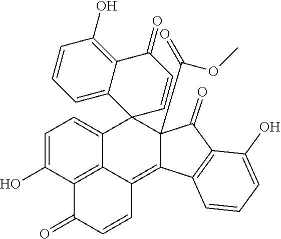 [SMILES: COC(=O)C12C(=O)c3c(O)cccc3C1=C1C=CC(=O)c3c(O)ccc(c31)C21C=CC(=O)c2c(O)cccc21] | Zhang, A. H. et. al. Org. Lett. 18, 6496-6499 (2016) which is incorporated herein by reference in its entirety. |
| JC-171 (5-chloro-N-[2- (4-hydroxysulfamoyl- phenyl)- ethyl]-2-methoxy- benzamide)) | 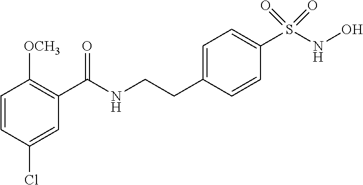 [SMILES: COc1ccc(Cl)cc1C(=O)NCCc1ccc(S(=O)(=O)NO)cc1] | Guo, C. etal. ACS Chem. Neurosci. 8, 2194-2201 (2017), which is incorporated herein by reference in its entirety. |
| INF39 | 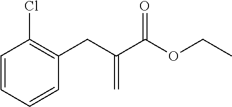 | Cocco, M. et al. J. Med. Chem. 60, 3656-3671 (2017), which is incorporated herein by reference in its entirety. |
| BOT-4-one | 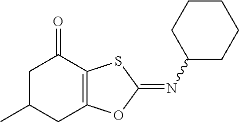 [SMILES: CC1CC(=O)c2sc(=NC3CCCCC3)oc2C1] | Kim, B.-H. et al. Exp. Mol. Med. 43, 313-321 (2011), which is incorporated herein by reference in its entirety. |
| β-hydroxybutyrate | | Youm, Y.-H. et al. Nat. Med. 21, 263-269 (2015), which is incorporated herein by reference in its entirety. |
| Quercetin | | Ruiz-Miyazawa, K. W. et al. Inflammopharmacology https://doi.org/10.1007/s107 87-017-0356-x (2017), which is incorporated herein by reference in its entirety. |
| Ibrutinib | 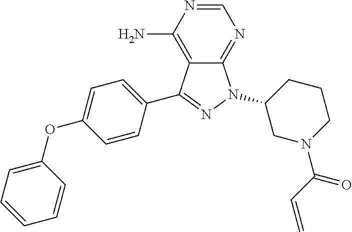 [SMILES: C=CC(=O)N1CCC[C@@H](n2nc(-c3ccc(Oc4ccccc4)cc3)c3c(N)ncnc32)C1] | Ito, M. et al. Nat. Commun. 6, 1-11 (2015), which is incorporated herein by reference in its entirety. |
| 2-aminoethoxy- diphenylborate (2-APB) | | Baldwin, A. G et al. Cell Chem. Biol. 24, 1321- 1335.e5 (2017), which is incorporated herein by reference in its entirety. |

In some embodiments, the NLRP3 antagonist is selected from the compound(s) recited and/or depicted in Perregaux, D. G. et al. J. Pharmacal. Exp. Ther. 299, 187-197 (2001), which is incorporated herein by reference in its entirety. For example, the NLRP3 antagonist is CP-446,773.

In some embodiments, the NLRP3 antagonist is selected from the compound(s) recited and/or depicted in Hill, J. R. et. al. Chem Med Chem 12, 1449-1457 (2017), which is incorporated herein by reference in its entirety.

In some embodiments, the NLRP3 antagonist is selected from the compound(s) recited and/or depicted in: Cocco, M. et, al. J. Med. Chem. 57, 10366-10382 (2014) and Cocco, M. et. al. Chem Med Chem 11, 1790-1803 (2016), both of which are incorporated herein by reference in their entirety. For example, the NLRP3 antagonist is INF58 or INF39.

In some embodiments, the NLRP3 antagonist is selected from the compound(s) recited and/or depicted in Baldwin, A. G. et al. Cell Chem. Biol. 24, 1321-1335.e5 (2017), which is incorporated herein by reference in its entirety. For example, the NLRP3 antagonist is a compound having the following structure:

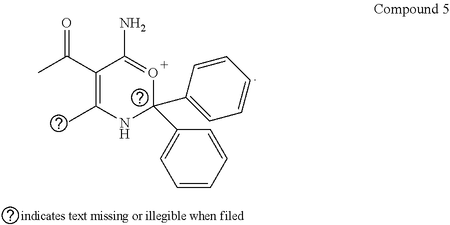

Exhibit A discloses NLRP3 antagonists and refers to NLRP3 antagonists in references cited therein, and is incorporated herein in its entirety by reference.

In some embodiments, the NLRP3 antagonist is a compound described in International application publication WO 2016/131098, incorporated by reference herein. For example, in some embodiments, the NLRP3 antagonist is a compound of formula (101A), having substituents as defined in claim 1, or a pharmaceutically acceptable salt, solvate or prodrug thereof, i.e. a compound of:

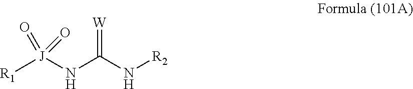

wherein,
W is selected from O, S and Se;
J is selected from S and Se;
R₁ is selected from the group consisting of cycloalkyl, aryl, heteroaryl and heterocyclyl,
all of which may be optionally substituted;
R₂ is selected from the group consisting of cycloalkyl, aryl, heteroaryl and heterocyclyl, all of which may be optionally substituted; and
both R₁ is directly bonded to J and R₂ is directly bonded to the adjacent nitrogen, via a carbon atom.

In some embodiments, NLRP3 antagonists which make up part of this invention include exemplary compounds in Table 2 on pages 285-315 of WO 2016/131098. In some embodiments, NLRP3 antagonists which make up part of this invention include the compounds of claim 36 of WO 2016/131098.

Embodiments of the invention include NLRP3 antagonists specifically defined in WO 2016/131098, for example the compounds in Table 2 on pages 285-315, in combination with an anti-TNFα agent; preferably in combination with Infliximab, Etanercept, Certolizumab pegol, Golimumab or Adalimumab; more preferably in combination with Adalimumab. Preferably a combination of any one of the compounds of claim 36 of WO 2016/131098 in combination with Adalimumab.

In some embodiments, the NLRP3 antagonist is a compound described in International application publication WO 2017/140778, incorporated by reference herein. For example, a compound of formula (102A), having substituents as defined in claim 1, or a pharmaceutically acceptable salt, solvate or prodrug thereof, i.e. a compound of:

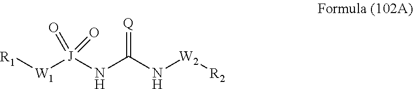

wherein Q is selected from O, S and Se;
J is S or Se;
W₁ and W₂, when present, are independently selected from N and C;
R₁ and R₂ are independently selected from the group consisting of hydrogen, C₁-C₁₂ alkyl, C₂-C₁₂ alkenyl, C₂-C₁₂ alkynyl, aryl, heterocyclyl, heteroaryl, cycloalkyl, cycloalkenyl, amino, amido, alkylthio, acyl, arylalkyl and acylamido, all of which may be optionally substituted; and
   wherein at least one of W₁ and W₂ is present and is a nitrogen atom and when R₁ or R₂ are cyclic then the respective W₁ or W₂ may form part of the ring structure.

In some embodiments, NLRP3 antagonists which make up part of this invention include exemplary compounds described in paragraph [0097] on pages 22-26, paragraph [00103] on pages 28-39, paragraph [00107] on pages 41-45, paragraph [00111] on pages 46-54, paragraph [00115] on pages 55-56, paragraph [00120] on pages 58-61, paragraph [00121] on pages 62, and Examples 1-43 on pages 105-126 of WO 2017/140778.

Embodiments of the invention include NLRP3 antagonists specifically defined in WO 2017/140778, for example the compounds described in paragraph [0097] on pages 22-26, paragraph [00103] on pages 28-39, paragraph [00107] on pages 41-45, paragraph [00111] on pages 46-54, paragraph [00115] on pages 55-56, paragraph [00120] on pages 58-61, paragraph [00121] on pages 62, and in Examples 1-43 on pages 105-126, in combination with an anti-TNFα agent; preferably in combination with Infliximab, Etanercept, Certolizumab pegol, Golimumab or Adalimumab; more preferably in combination with Adalimumab.

In some embodiments, the NLRP3 antagonist is a compound described in International application publication WO2018/215818, incorporated by reference herein. For example, a compound of Formula (103A), having substituents as defined in claim 1, or a pharmaceutically acceptable salt, solvate or prodrug thereof, i.e. a compound of:

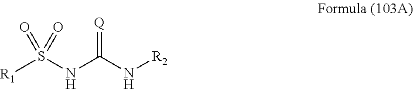

wherein:
Q is O or S;
R₁ a cyclic group substituted with at least one group X, wherein R₁ may optionally be further substituted;
X is any group comprising a carbonyl group; and
R₂ a cyclic group substituted at the alpha-position, wherein R₂ may optionally be further substituted.

In some embodiments, NLRP3 antagonists which make up part of this invention include exemplary compounds described on page 13, line 8 to page 17, line 2; page 17, line 21 to page 20, line 7; page 21, line 8 to page 22, line 5; page 29, line 8 to page 34, line 7, and in Examples 1-31 on pages 66-83 of WO2018/215818. In some embodiments, NLRP3 antagonists which make up part of this invention include the compounds of claim 13 of WO2018/215818.

Embodiments of the invention include NLRP3 antagonists specifically defined in WO2018/215818, for example the compounds described on page 13, line 8 to page 17, line 2; page 17, line 21 to page 20, line 7; page 21, line 8 to page 22, line 5; page 29, line 8 to page 34, line 7, and in Examples 1-31 on pages 66-83 in combination with an anti-TNFα agent; preferably in combination with Infliximab, Etanercept, Certolizumab pegol, Golimumab or Adalimumab; more preferably in combination with Adalimumab. Preferably a combination of any one of the compounds of claim 13 of WO2018/215818 in combination with Adalimumab.

In some embodiments, the NLRP3 antagonist is a compound described in International application publication WO2019/008025, incorporated by reference herein. For example, a compound of formula (104A), having substituents as defined in claim 1, or a pharmaceutically acceptable salt, solvate or prodrug thereof, i.e. a compound of:

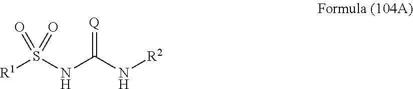

wherein:
Q is selected from O or S;
R¹ is a non-aromatic heterocyclic group comprising at least one ring nitrogen atom, wherein R¹ is attached to the sulfur atom of the sulfonylurea group by a ring carbon atom, and wherein R¹ may optionally be substituted; and
R² is a cyclic group substituted at the α-position, wherein R² may optionally be further substituted.

In some embodiments, NLRP3 antagonists which make up part of this invention include exemplary compounds described on page 39, line 4 to page 77, line 3 and in Examples 1-195 on pages 221-320 of WO2019/008025. In some embodiments, NLRP3 antagonists which make up part of this invention include the compounds of claim 16 of WO2019/008025.

Embodiments of the invention include NLRP3 antagonists specifically defined in WO2019/008025, for example the compounds described on on page 39, line 4 to page 77, line 3 and in Examples 1-195 on pages 221-320 in combination with an anti-TNFα agent; preferably in combination with Infliximab, Etanercept, Certolizumab pegol, Golimumab or Adalimumab; more preferably in combination with Adalimumab. Preferably a combination of any one of the compounds of claim 16 of WO2019/008025 in combination with Adalimumab.

In some embodiments, the NLRP3 antagonist is a compound described in International application publication WO 2019/034696, incorporated by reference herein. For example, a compound of formula (105A), having substituents as defined in claim 1, or a pharmaceutically acceptable salt, solvate or prodrug thereof, i.e. a compound of:

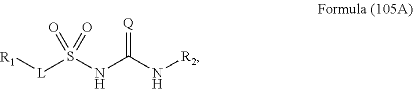

wherein
Q is selected from O or S;
L is a saturated or unsaturated C₁-C₁₂ hydrocarbylene group, wherein the hydrocarbylene group may be straight-chained or branched, or be or include cyclic groups, wherein the hydrocarbylene group may optionally be substituted, and wherein the hydrocarbylene group may optionally include one or more heteroatoms N, O or S in its carbon skeleton;
R1 is —NR³R4, —OR5, —(C═NR⁶)R7, —(CO)R8, —CN, —N3, a quaternary ammonium group or an optionally substituted heterocycle;
R3, R4, R5, R6, R7 and R8 are each independently hydrogen or a saturated or unsaturated C₁-C₁₀ hydrocarbyl group, wherein the hydrocarbyl group may be straight chained or branched, or be or include cyclic groups, wherein the hydrocarbyl group may optionally be substituted, and wherein the hydrocarbyl group may optionally include one or more heteroatoms N, O or S in its carbon skeleton;
   wherein optionally L and R3, or L and R4, or R3 and R4 together with the nitrogen atom to which they are attached may form a 3- to 12-membered saturated or unsaturated cyclic group, wherein the cyclic group may optionally be substituted;
   wherein optionally L and R5 together with the oxygen atom to which they are attached may form a S- to 12-membered saturated or unsaturated cyclic group, wherein the cyclic group may optionally be substituted;
   wherein optionally L and R6, or L and R7, or R6 and R7 together with the —(C═N)— group to which they are attached may form a 3- to 12-membered saturated or unsaturated cyclic group, wherein the cyclic group may optionally be substituted;
   wherein optionally L and R8 together with the —(C═O)— group to which they are attached may form a 3- to 12-membered saturated or unsaturated cyclic group, wherein the cyclic group may optionally be substituted;
   R2 is a cyclic group substituted at the α-position, wherein R2 may optionally be further substituted;
   provided that the atom of L which is attached to the sulfur atom of the sulfonylurea group is a carbon atom and is not a ring atom of a heterocyclic or aromatic group.

In some embodiments, NLRP3 antagonists which make up part of this invention include exemplary compounds described on page 59, line 12 to page 101, line 3 and in Examples 1-112 on pages 186-241 of WO 2019/034696. In some embodiments, NLRP3 antagonists which make up part of this invention include the compounds of claim 15 of WO 2019/034696.

Embodiments of the invention include NLRP3 antagonists specifically defined in WO 2019/034696, for example the compounds described on page 59, line 12 to page 101, line 3 and in Examples 1-112 on pages 186-241 in combination with an anti-TNFα agent; preferably in combination with Infliximab, Etanercept, Certolizumab pegol, Golimumab or Adalimumab; more preferably in combination with Adalimumab. Preferably a combination of any one of the compounds of claim 15 of WO 2019/034696 in combination with Adalimumab.

In some embodiments, the NLRP3 antagonist is a compound described in International application publication WO 2019/008029, incorporated by reference herein. For example, a compound of formula (106A), having substituents as defined in claim 1, or a pharmaceutically acceptable salt, solvate or prodrug thereof, i.e. a compound of:

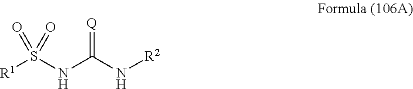

wherein:
Q is O or S;
R¹ is a 6-membered heteroaryl group containing at least one nitrogen atom in the 6-membered ring structure, wherein R¹ may optionally be substituted; and R² is a cyclic group substituted at the α-position, wherein R² may optionally be further substituted; including

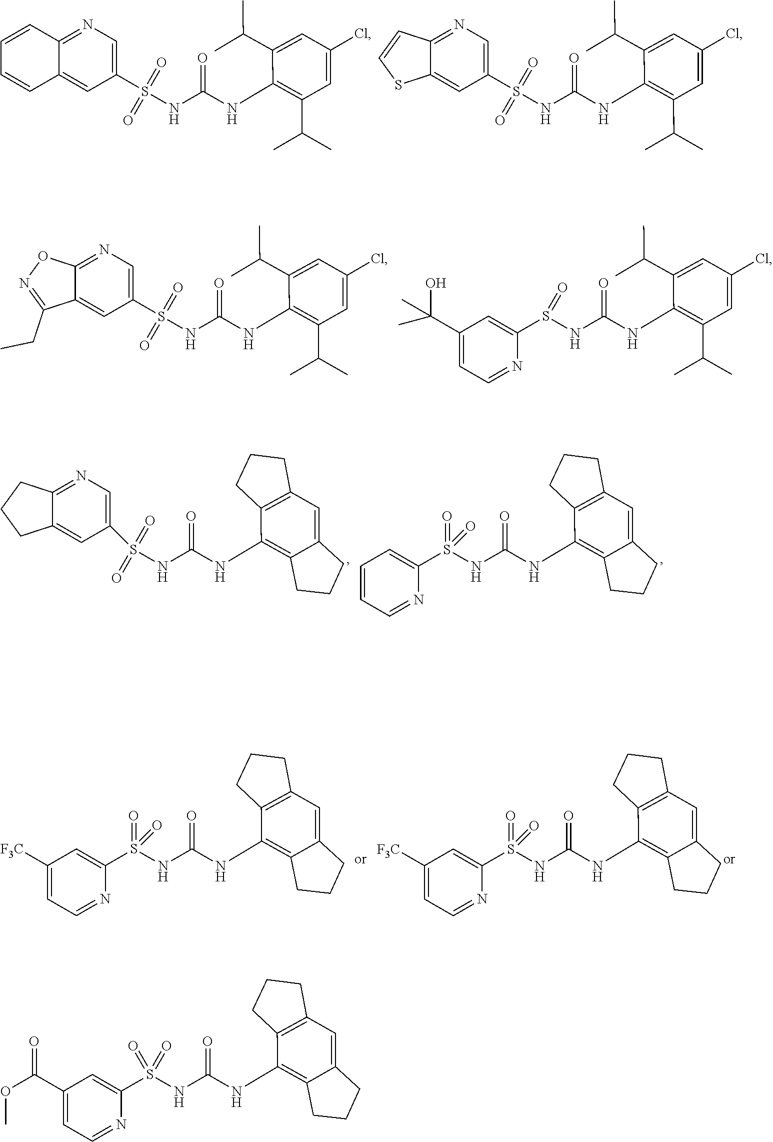

In some embodiments, NLRP3 antagonists which make up part of this invention include exemplary compounds described on page 54, line 1 to page 83, line 3 and in Examples 1-60 on pages 145-176 of WO 2019/008029. In some embodiments, NLRP3 antagonists which make up part of this invention include exemplary compounds of claim 16 of WO 2019/008029.

Embodiments of the invention include NLRP3 antagonists specifically defined in WO 2019/008029, for example the compounds described on page 54, line 1 to page 83, line 3 and in Examples 1-60 on pages 145-176 in combination with an anti-TNFα agent; preferably in combination with Infliximab, Etanercept, Certolizumab pegol, Golimumab or Adalimumab; more preferably in combination with Adalimumab. Preferably, a combination of a compounds of claim 16 of WO 2019/008029 in combination with Adalimumab.

In some embodiments, the NLRP3 antagonist is a compound described in International application publication WO 2019/034693, incorporated by reference herein. For example, a compound of formula (107A), having substituents as defined in claim 1, or a pharmaceutically acceptable salt, solvate or prodrug thereof, i.e. a compound of:

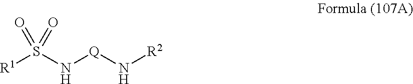

wherein:
Q is selected from O or S;
R¹ is an imidazolyl group, wherein the imidazolyl group is unsubstituted or substituted with one or more monovalent substituents; and
R² is a cyclic group substituted at the α-position, wherein R² may optionally be further substituted.

In some embodiments, NLRP3 antagonists which make up part of this invention include exemplary compounds described on page 43, line 13 to page 44, line 5 and in Examples 1-21 on pages 95-105 of WO 2019/034693. In some embodiments, NLRP3 antagonists which make up part of this invention include exemplary compounds of claim 16 of WO 2019/034693.

Embodiments of the invention include NLRP3 antagonists specifically defined in WO 2019/034693, for example the compounds described on page 54, line 1 to page 83, line 3 and in Examples 1-60 on pages 145-176 in combination with an anti-TNFα agent; preferably in combination with Infliximab, Etanercept, Certolizumab pegol, Golimumab or Adalimumab; more preferably in combination with Adalimumab. Preferably, a combination of a compounds of claim 16 of WO 2019/034693 in combination with Adalimumab.

In some embodiments, the NLRP3 antagonist is a compound described in International application publication WO 2019/034692, incorporated by reference herein. For example, a compound of formula (108A), having substituents as defined in claim 1, or a pharmaceutically acceptable salt, solvate or prodrug thereof, i.e. a compound of:

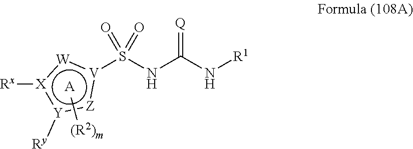

wherein:
Q is selected from O or S;
V, X and Y are each independently selected from C and N, and W and Z are each independently selected from N, O, S, NH and CH, provided that at least one of V, W, X, Y and Z is N, O, S or NH;
R^{X} and R^{Y} are each independently any saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
optionally R^{X} and R^{Y} together with the atoms X and Y to which they are attached may form a 3- to 12-membered saturated or unsaturated cyclic group, wherein the cyclic group may optionally be substituted;
R¹ is a cyclic group substituted at the α-position, wherein R¹ may optionally be further substituted;
m is 0, 1 or 2;
each R² is independently a halo, —OH, —NO₂, —NH₂, —N₃, —SH, or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
optionally R^{X} and any R² attached to W may together with the atoms W and X to which they are attached form a 3- to 12-membered saturated or unsaturated cyclic group, wherein the cyclic group may optionally be substituted; and
optionally R^{Y} and any R² attached to Z may together with the atoms Y and Z to which they are attached form a 3- to 12-membered saturated or unsaturated cyclic group, wherein the cyclic group may optionally be substituted;
provided that at least one of R^{X} and R^{Y} comprises a nitrogen atom;
including:

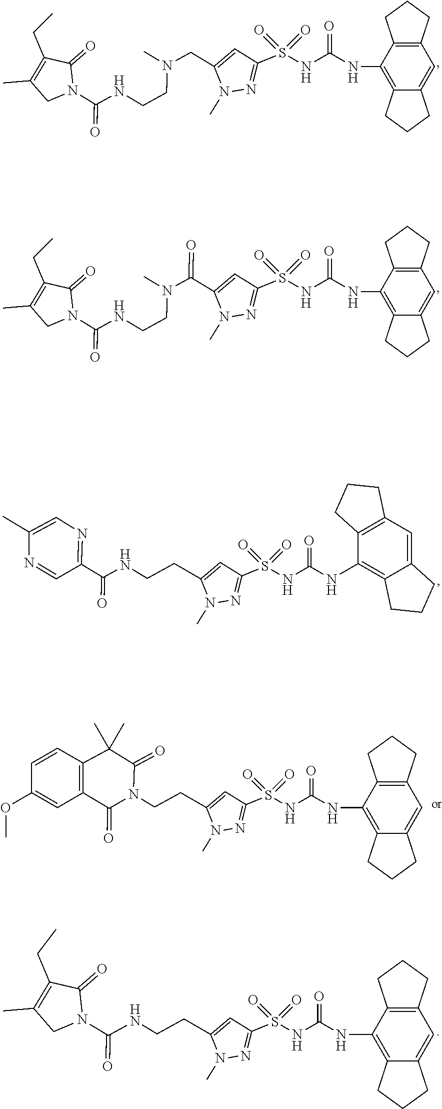 [SMILES: CCC1=C(C)CN(C(=O)NCCN(C)Cc2cc(S(=O)(=O)NC(=O)Nc3c4c(cc5c3CCC5)CCC4)nn2C)C1=O ; CCC1=C(C)CN(C(=O)NCCN(C)C(=O)c2cc(S(=O)(=O)NC(=O)Nc3c4c(cc5c3CCC5)CCC4)nn2C)C1=O ; Cc1cnc(C(=O)NCCc2cc(S(=O)(=O)NC(=O)Nc3c4c(cc5c3CCC5)CCC4)nn2C)cn1 ; COc1ccc2c(c1)C(=O)N(CCc1cc(S(=O)(=O)NC(=O)Nc3c4c(cc5c3CCC5)CCC4)nn1C)C(=O)C2(C)C ; CCC1=C(C)CN(C(=O)NCCc2cc(S(=O)(=O)NC(=O)Nc3c4c(cc5c3CCC5)CCC4)nn2C)C1=O]

In some embodiments, NLRP3 antagonists which make up part of this invention include exemplary compounds described on page 62, line 1 to page 76, line 5 and in Examples 1-187 on pages 230-336 of WO 2019/034692. In some embodiments, NLRP3 antagonists which make up part of this invention include exemplary compounds of claim 20 of WO 2019/034692.

Embodiments of the invention include NLRP3 antagonists specifically defined in WO 2019/034692, for example the compounds described on page 62, line 1 to page 76, line 5 and in Examples 1-187 on pages 230-336 in combination with an anti-TNFα agent; preferably in combination with Infliximab, Etanercept, Certolizumab pegol, Golimumab or Adalimumab; more preferably in combination with Adalimumab. Preferably, a combination of a compounds of claim 20 of WO 2019/034692 in combination with Adalimumab.

In some embodiments, the NLRP3 antagonist is a compound described in International application publication WO 2019/034690, incorporated by reference herein. For example, a compound of formula (109A), having substituents as defined in claim 1, or a pharmaceutically acceptable salt, solvate or prodrug thereof, i.e. a compound of:

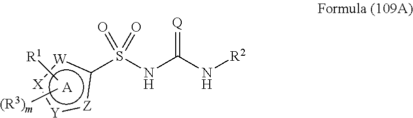

wherein:
Q is selected from O or S;
W, X, Y and Z are each independently N, O, S, NH or CH, wherein at least one of W, X,
Y and Z is N or NH;
R¹ is a monovalent group comprising at least one nitrogen atom, wherein —R¹ contains from 1 to 7 atoms other than hydrogen or halogen; or
R¹ is a divalent group comprising at least one nitrogen atom, wherein —R¹-contains from 1 to 7 atoms other than hydrogen or halogen, and wherein —R¹— is directly attached to any two adjacent W, X, Y or Z;
R² is a cyclic group substituted at the α-position, wherein R² may optionally be further substituted;
m is o, 1, 2 or 3;
each R³ is independently a halo, —OH, —NO₂, —NH₂, —N₃, —SH, or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton; and
wherein optionally any R³, and any two adjacent W, X, Y or Z, may together form a 3- to 12-membered saturated or unsaturated cyclic group fused to ring A, wherein the cyclic group fused to ring A may optionally be substituted.

In some embodiments, NLRP3 antagonists which make up part of this invention include exemplary compounds described on page 59, line 1 to page 75, line 5 and in Examples 1-210 on pages 282-394 of WO 2019/034690. In some embodiments, NLRP3 antagonists which make up part of this invention include exemplary compounds of claim 21 of WO 2019/034690.

Embodiments of the invention include NLRP3 antagonists specifically defined in WO 2019/034690, for example the compounds described on page 59, line 1 to page 75, line 5 and in Examples 1-210 on pages 282-394 in combination with an anti-TNFα agent;

preferably in combination with Infliximab, Etanercept, Certolizumab pegol, Golimumab or Adalimumab; more preferably in combination with Adalimumab. Preferably a compound of claim 21 of WO 2019/034690 in combination with Adalimumab.

In some embodiments, the NLRP3 antagonist is a compound described in International application publication WO 2019/034688, incorporated by reference herein. For example, a compound of formula (110A), having substituents as defined in claim 1, or a pharmaceutically acceptable salt, solvate or prodrug thereof, i.e. a compound of:

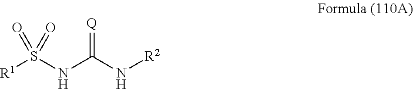

wherein:
Q is selected from O or S;
R¹ is a 5-membered heteroaryl group substituted with at least one group R^{X}, wherein R^{X} is any group comprising an amide group, wherein the 5-membered heteroaryl group may optionally be further substituted; and
R² is a cyclic group substituted at the α-position, wherein R² may optionally be further substituted;
including:

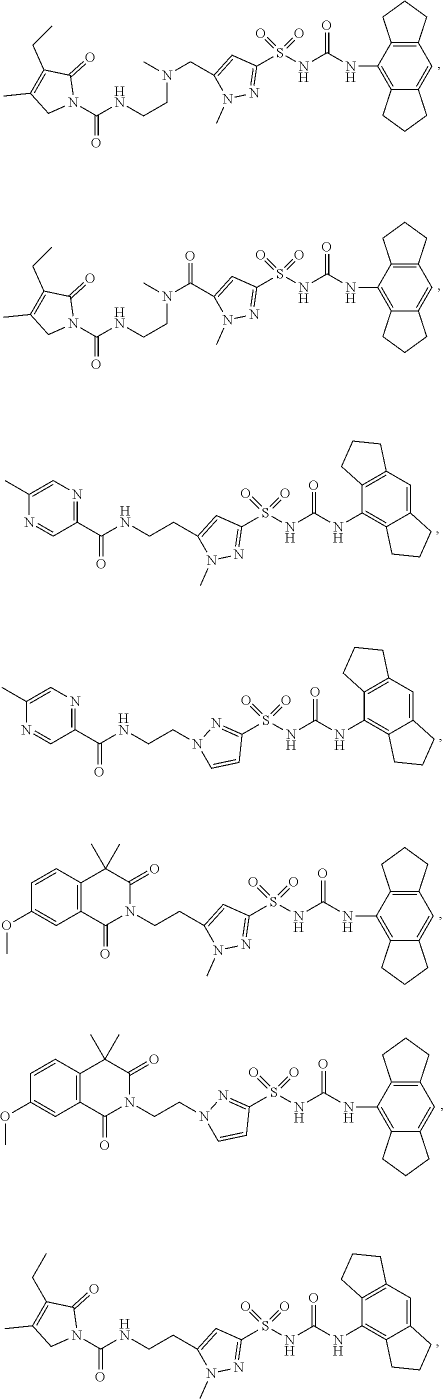

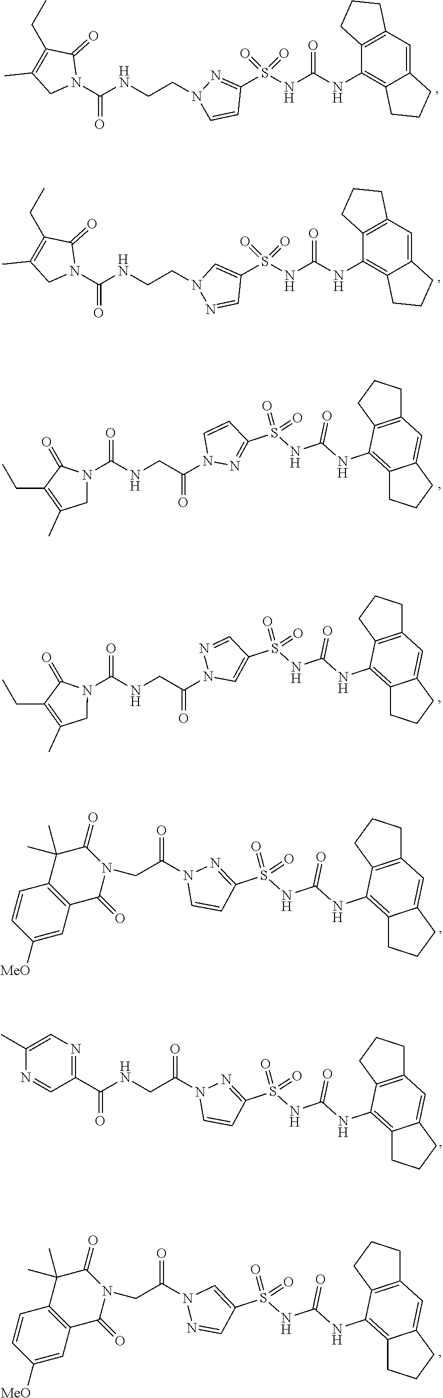

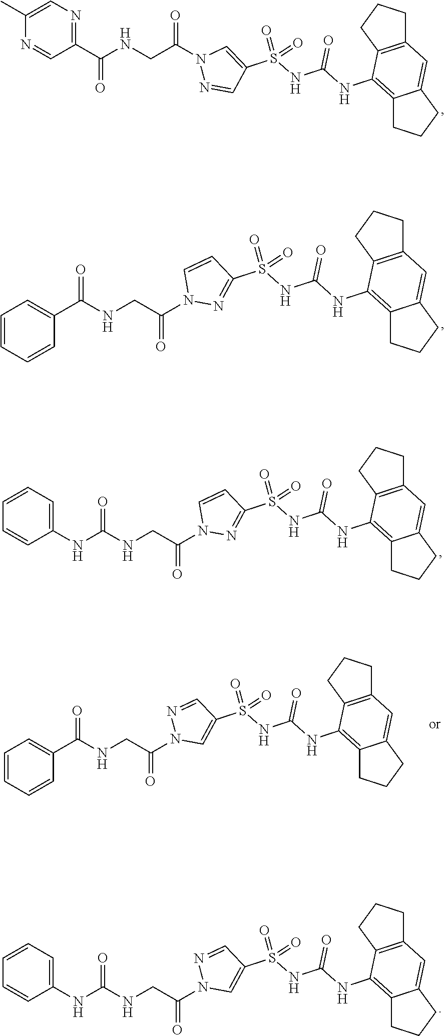 [SMILES: Cc1cnc(C(=O)NCC(=O)n2cc(S(=O)(=O)NC(=O)Nc3c4c(cc5c3CCC5)CCC4)cn2)cn1 ; O=C(Nc1c2c(cc3c1CCC3)CCC2)NS(=O)(=O)c1ccn(C(=O)CNC(=O)c2ccccc2)n1 ; O=C(NCC(=O)n1ccc(S(=O)(=O)NC(=O)Nc2c3c(cc4c2CCC4)CCC3)n1)Nc1ccccc1 ; O=C(Nc1c2c(cc3c1CCC3)CCC2)NS(=O)(=O)c1cnn(C(=O)CNC(=O)c2ccccc2)c1 ; O=C(NCC(=O)n1cc(S(=O)(=O)NC(=O)Nc2c3c(cc4c2CCC4)CCC3)cn1)Nc1ccccc1]

In some embodiments, NLRP3 antagonists which make up part of this invention include exemplary compounds described on page 61, line 1 to page 69, line 3 and in Examples 1-84 on pages 149-198 of WO 2019/034688.

Embodiments of the invention include NLRP3 antagonists specifically defined in WO 2019/034688, for example the compounds described on page 61, line 1 to page 69, line 3 and in Examples 1-84 on pages 149-198 in combination with an anti-TNFα agent; preferably in combination with Infliximab, Etanercept, Certolizumab pegol, Golimumab or Adalimumab; more preferably in combination with Adalimumab.

In some embodiments, the NLRP3 antagonist is a compound described in International application publication WO 2019/034686, incorporated by reference herein. For example, a compound of formula (111A), having substituents as defined in claim 1, or a pharmaceutically acceptable salt, solvate or prodrug thereof, i.e. a compound of:

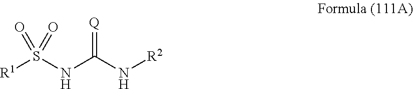

wherein:
Q is selected from O or S;
R¹ is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton; and
R² is a cyclic group substituted at the α-position with a monovalent heterocyclic group or a monovalent aromatic group, wherein a ring atom of the heterocyclic or aromatic group is directly attached to a ring atom of the cyclic group, wherein the heterocyclic or aromatic group may optionally be substituted, and wherein the cyclic group may optionally be further substituted.

In some embodiments, NLRP3 antagonists which make up part of this invention include exemplary compounds described on page 40, line 7 to page 67, line 5 and in Examples 1-323 on pages 149-354 of WO 2019/034686. In some embodiments, NLRP3 antagonists which make up part of this invention include exemplary compounds of claim 10 of WO 2019/034686.

Embodiments of the invention include NLRP3 antagonists specifically defined in WO 2019/034686, for example the compounds described on page 40, line 7 to page 67, line 5 and in Examples 1-323 on pages 149-354 in combination with an anti-TNFα agent; preferably in combination with Infliximab, Etanercept, Certolizumab pegol, Golimumab or Adalimumab; more preferably in combination with Adalimumab. Preferably, a compound of claim 10 of WO 2019/034686 in combination with Adalimumab.

In some embodiments, the NLRP3 antagonist is a compound described in International application publication WO 2019/092172, incorporated by reference herein. For example, a compound of formula (112A), having substituents as defined in claim 1, or a pharmaceutically acceptable salt, solvate or prodrug thereof, i.e. a compound of:

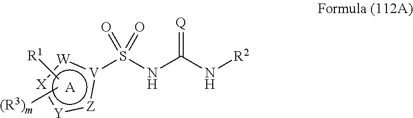

wherein:
Q is selected from O or S;
V is independently selected from C and N, and W, X, Y and Z are each independently selected from N, O, S, NH or CH, provided that at least one of V, W, X, Y and Z is N or NH;
R¹ is a monovalent group comprising a non-aromatic cyclic group;
R² is a 6-membered cyclic group substituted at the 2- and 4-positions, wherein the 6-membered cyclic group may optionally be further substituted;
m is o, 1, 2 or 3;
each R3 is independently a halo, —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton; and
wherein optionally any R³, and any two adjacent W, X, Y or Z, may together form a 4- to 12-membered saturated or unsaturated cyclic group fused to ring A, wherein the cyclic group fused to ring A may optionally be substituted.

In some embodiments, NLRP3 antagonists which make up part of this invention include exemplary compounds described on page 52, line 19 to page 57, line 3 and in Examples 1-21 on pages 116-125 of WO 2019/092172. In some embodiments, NLRP3 antagonists which make up part of this invention include exemplary compounds of claim 17 of WO 2019/092172.

Embodiments of the invention include NLRP3 antagonists specifically defined in WO 2019/092172, for example the compounds described on page 52, line 19 to page 57, line 3 and in Examples 1-21 on pages 116-125 in combination with an anti-TNFα agent; preferably in combination with Infliximab, Etanercept, Certolizumab pegol, Golimumab or Adalimumab; more preferably in combination with Adalimumab. Preferably, a compound claim 17 of WO 2019/092172 in combination with Adalimumab.

In some embodiments, the NLRP3 antagonist is a compound described in International application publication WO 2019/092171, incorporated by reference herein. For example, a compound of formula (113A), having substituents as defined in claim 1, or a pharmaceutically acceptable salt, solvate or prodrug thereof, i.e. a compound of:

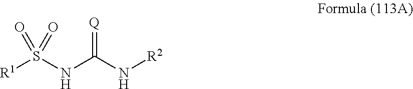

wherein:
Q is selected from O or S;
R¹ is a 5-membered heteroaryl group substituted with at least one group R^{X}, wherein R^{X} is any monovalent group comprising a heteroaryl group, and wherein the 5-membered heteroaryl group of R′ may optionally be further substituted; and
R² is a cyclic group substituted at the α-position, wherein R² may optionally be further substituted;
including:

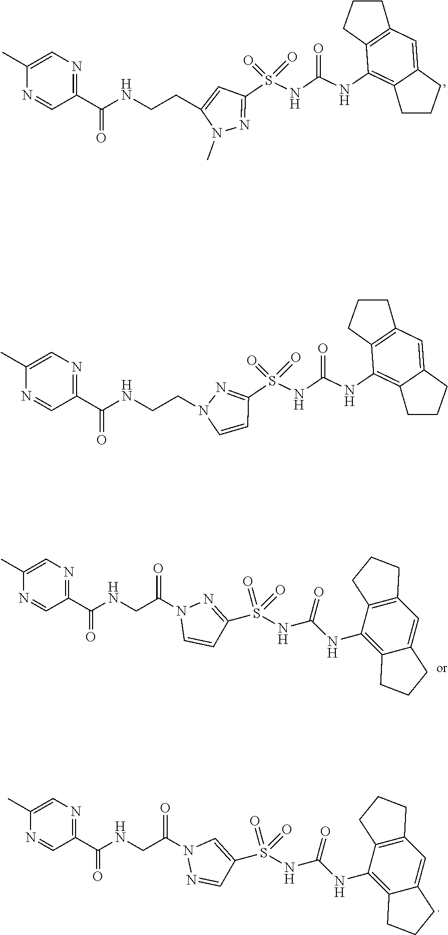 [SMILES: Cc1cnc(C(=O)NCCc2cc(S(=O)(=O)NC(=O)Nc3c4c(cc5c3CCC5)CCC4)nn2C)cn1 ; Cc1cnc(C(=O)NCCn2ccc(S(=O)(=O)NC(=O)Nc3c4c(cc5c3CCC5)CCC4)n2)cn1 ; Cc1cnc(C(=O)NCC(=O)n2ccc(S(=O)(=O)NC(=O)Nc3c4c(cc5c3CCC5)CCC4)n2)cn1 ; Cc1cnc(C(=O)NCC(=O)n2cc(S(=O)(=O)NC(=O)Nc3c4c(cc5c3CCC5)CCC4)cn2)cn1]

In some embodiments, NLRP3 antagonists which make up part of this invention include exemplary compounds described on page 48, lines 1-5 and in Examples 1-10 on pages 94-100 of WO 2019/092171. In some embodiments, NLRP3 antagonists which make up part of this invention include exemplary compounds of claim 18 of WO 2019/092171.

Embodiments of the invention include NLRP3 antagonists specifically defined in WO 2019/092171, for example the compounds described on page 48, lines 1-5 and in Examples 1-10 on pages 94-100 in combination with an anti-TNFα agent; preferably in combination with Infliximab, Etanercept, Certolizumab pegol, Golimumab or Adalimumab; more preferably in combination with Adalimumab. Preferably, a compound claim 18 of WO 2019/092171 in combination with Adalimumab.

In some embodiments, the NLRP3 antagonist is a compound described in International application publication WO 2019/092170, incorporated by reference herein. For example, a compound of formula (114A), having substituents as defined in claim 1, or a pharmaceutically acceptable salt, solvate or prodrug thereof, i.e. a compound of:

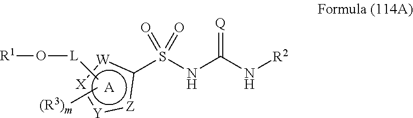

wherein:
Q is selected from O or S;
W, X, Y and Z are each independently N, O, S, NH or CH, wherein at least one of W, X,
Y and Z is N or NH;
L is a saturated or unsaturated hydrocarbylene group, wherein the hydrocarbylene group may be straight-chained or branched, or be or include cyclic groups, wherein the hydrocarbylene group may optionally be substituted, and wherein the hydrocarbylene group may optionally include one or more heteroatoms N, O or S in its carbon skeleton; R¹ is hydrogen or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
optionally L and R¹ together with the oxygen atom to which they are attached may form a 3- to 12-membered saturated or unsaturated cyclic group, wherein the cyclic group may optionally be substituted;
optionally R¹—O-L-, and any two adjacent W, X, Y or Z, may together form a 3- to 12-membered saturated or unsaturated cyclic group fused to ring A, wherein the cyclic group fused to ring A may optionally be substituted;
R² is a cyclic group substituted at the α-position, wherein R² may optionally be further substituted;
each R3 is independently a halo, —OH, —NO₂, —NH₂, —N₃, —SH, or a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton;
m is 0, 1, 2 or 3; and
optionally any R³, and any two adjacent W, X, Y or Z, may together form a 3- to 12-membered saturated or unsaturated cyclic group fused to ring A, wherein the cyclic group fused to ring A may optionally be substituted;
provided that any atom of L or R¹ that is directly attached to W, X, Y or Z is not the same atom of L or R¹ that is directly attached to the oxygen atom of R¹—O-L-;
including:

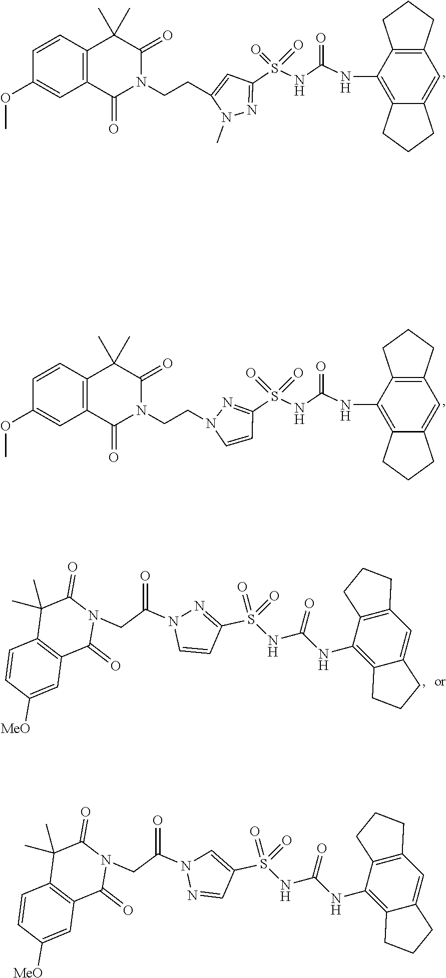 [SMILES: COc1ccc2c(c1)C(=O)N(CCc1cc(S(=O)(=O)NC(=O)Nc3c4c(cc5c3CCC5)CCC4)nn1C)C(=O)C2(C)C ; COc1ccc2c(c1)C(=O)N(CCn1ccc(S(=O)(=O)NC(=O)Nc3c4c(cc5c3CCC5)CCC4)n1)C(=O)C2(C)C ; COc1ccc2c(c1)C(=O)N(CC(=O)n1ccc(S(=O)(=O)NC(=O)Nc3c4c(cc5c3CCC5)CCC4)n1)C(=O)C2(C)C ; COc1ccc2c(c1)C(=O)N(CC(=O)n1cc(S(=O)(=O)NC(=O)Nc3c4c(cc5c3CCC5)CCC4)cn1)C(=O)C2(C)C]

In some embodiments, NLRP3 antagonists which make up part of this invention include exemplary compounds described on page 58, lines 1 to page 67, line 3 and in Examples 1-107 on pages 189-246 of WO 2019/092170. In some embodiments, NLRP3 antagonists which make up part of this invention include exemplary compounds of claim 18 of WO 2019/092170.

Embodiments of the invention include NLRP3 antagonists specifically defined in WO 2019/092170, for example the compounds described on page 58, lines 1 to page 67, line 3 and in Examples 1-107 on pages 189-246 in combination with an anti-TNFα agent; preferably in combination with Infliximab, Etanercept, Certolizumab pegol, Golimumab or Adalimumab; more preferably in combination with Adalimumab. Preferably, a compound claim 18 of WO 2019/092170 in combination with Adalimumab.

In some embodiments, the NLRP3 antagonist is a compound described in International application publication WO 2019/034697, incorporated by reference herein. For example, a compound of formula (115A having substituents as defined in claim 1, or a pharmaceutically acceptable salt, solvate or prodrug thereof, i.e. a compound of:

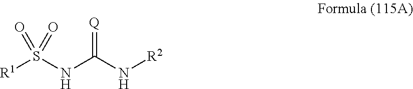

wherein:
Q is selected from O or S;
R¹ is a saturated or unsaturated C1-C15 hydrocarbyl group, wherein the hydrocarbyl group may be straight-chained or branched, or be or include cyclic groups, wherein the hydrocarbyl group may optionally be substituted, and wherein the atom of R¹ which is attached to the sulfur atom of the sulfonylurea group is not a ring atom of a cyclic group; and
R² is a cyclic group substituted at the α-position, wherein R² may optionally be further substituted.

In some embodiments, NLRP3 antagonists which make up part of this invention include exemplary compounds described on page 50, lines 7 to page 53, line 3 and in Examples 1-40 on pages 99-119 of WO 2019/034697. In some embodiments, NLRP3 antagonists which make up part of this invention include exemplary compounds of claim 13 of WO 2019/034697.

Embodiments of the invention include NLRP3 antagonists specifically defined in WO 2019/034697, for example the compounds described on page 50, lines 7 to page 53, line 3 and in Examples 1-40 on pages 99-119 in combination with an anti-TNFα agent; preferably in combination with Infliximab, Etanercept, Certolizumab pegol, Golimumab or Adalimumab; more preferably in combination with Adalimumab. Preferably, a compound claim 13 of WO 2019/034697 in combination with Adalimumab.

In some embodiments, the NLRP3 antagonist is a compound described in International application publication WO 2019/068772, incorporated by reference herein. For example, a compound of formula (116A), having substituents as defined in claim 1, or a pharmaceutically acceptable salt, solvate or prodrug thereof, i.e. a compound of:

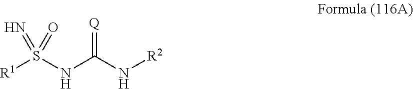

wherein:
Q is selected from O or S;
R¹ is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton; and
R² is a cyclic group substituted at the α-position, wherein R² may optionally be further substituted.

In some embodiments, NLRP3 antagonists which make up part of this invention include exemplary compounds described on page 44, lines 11 to page 52, line 10 and in Examples 1-28 on pages 117-158 of WO 2019/068772. In some embodiments, NLRP3 antagonists which make up part of this invention include exemplary compounds of claim 13 of WO 2019/068772.

Embodiments of the invention include NLRP3 antagonists specifically defined in WO 2019/068772, for example the compounds described on page 44, lines 11 to page 52, line 10 and in Examples 1-28 on pages 117-158 in combination with an anti-TNFα agent; preferably in combination with Infliximab, Etanercept, Certolizumab pegol, Golimumab or Adalimumab; more preferably in combination with Adalimumab. Preferably, a compound claim 13 of WO 2019/068772 in combination with Adalimumab.

In some embodiments, the NLRP3 antagonist is a compound described in International application publication WO18136890, incorporated by reference herein. For example, a compound of formula

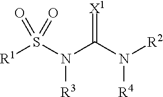

as defined in claim 1 of WO18136890.

In some embodiments, NLRP3 antagonists which make up part of this invention include exemplary compounds described in paragraphs [00461] to [00464], and examples 1 to 88 of WO18136890. In some embodiments, NLRP3 antagonists which make up part of this invention include exemplary compounds of claims 65 to 68 of WO18136890.

Embodiments of the invention include NLRP3 antagonists specifically defined in WO 2019/068772, for example the compounds described on page 44, lines 11 to page 52, line 10 and in Examples 1-28 on pages 117-158 in combination with an anti-TNFα agent; preferably in combination with Infliximab, Etanercept, Certolizumab pegol, Golimumab or Adalimumab; more preferably in combination with Adalimumab. Preferably, a compound claims 65 to 68 of WO18136890 in combination with Adalimumab.

In some embodiments, the NLRP3 antagonist is a compound described in International application publication WO18015445, incorporated by reference herein. For example, a compound of formula as defined in claim 1, i.e. a compound of formula (117A), or a pharmaceutically acceptable salt, solvate or prodrug thereof:

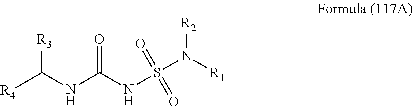

in which:
R1 and R2, together with the nitrogen atom to which they are attached to, form a 5 membered monocyclic heteroaryl ring system, wherein the monocyclic heteroaryl ring system, in addition to the nitrogen atom to which R1 and R2 are attached to, optionally comprises 1, 2 or 3 further heteroatoms independently selected from oxygen, nitrogen and sulfur; or
R1 and R2, together with the nitrogen atom to which they are attached to, form a 8, 9 or 10 membered bicyclic heteroaryl ring system, wherein the bicyclic heteroaryl ring system, in addition to the nitrogen atom to which R1 and R2 are attached to, optionally comprises 1, 2 or 3 further heteroatoms independently selected from oxygen, nitrogen and sulfur;
wherein said monocyclic heteroaryl ring system or said bicyclic heteroaryl ring system is optionally substituted by 1, 2, or 3 substituents independently selected from (1-6C)alkyl, (C₂-C₆)alkenylene, (C₂-C₆)alkynylene, (C₃-C₈)cycloalkyl, halo, nitro, cyano, CF3, oxo, OR5, N(R6)(R7), NR⁵C(O)R6, NR⁶S(O)₂R8, N(R5)C(O)N(R6)(R7), S(O)2R8, S(O)R8, S(O)(NR⁵)(R8), S(O)2N(R6)(R7), C(O)OR5, C(O)N(R6)(R7), C(O)R8, C(NORS)(R8), OC(O)N(R6)(R7), OC(O)R8, phenyl, a 5 or 6 membered monocyclic heteroaryl ring system comprising 1, 2 or 3 heteroatoms independently selected from oxygen, nitrogen and sulfur, and a 3, 4, 5, or 6 membered monocyclic heterocyclyl ring system comprising 1 or 2 heteroatoms independently selected from oxygen, nitrogen and sulfur, wherein said (C₁-C₆)alkyl, (C₂-C₆)alkenylene, (C₂-C₆)alkynylene, (C₃-C₈)cycloalkyl, phenyl, 5 or 6 membered monocyclic heteroaryl ring system or 3, 4, 5, or 6 membered monocyclic heterocyclyl ring system is optionally substituted by 1, 2, 3 or 4 substituents independently selected from halo, cyano, oxo, CF3, OR5, N(R6)(R7), NR5C(O)Rs, SR5, C(O)N(R6)(R7), S(O)₂R8, SORB, S(O)₂N(R5)(R6) and NR⁵S(O)₂R6;

R3 and R4, together with the carbon atom to which they are attached to, form a 12, 13, 14, 15 or 16 membered tricyclic partially unsaturated heterocyclic or carbocyclic ring system, wherein said tricyclic ring system is optionally substituted by 1, 2, 3 or 4 substituents independently selected from (C₁-C₆)alkyl, (C₂-C₆)alkenylene, (C₂-C₆)alkynylene, (C₃-C₈)cycloalkyl, (C₁-C₃)alkoxy, halo, oxo, hydroxy, cyano, amino, (C₁-C₃)alkylamino, di-[(C₁-C₃)alkyl]-amino, CF3, OCF3, S(O)2CH3, S(O)CH3, S(O)2NH2, S(O)2NHCH3, S(O)2N(CH3)2, NHS(O)2CH3 and N(CH3)S(O)2CH3;

R5, R6 and R7 are each independently selected from H, (C₁-C₆)alkyl, CF3, phenyl, a 5 or 6 membered monocyclic heteroaryl ring system comprising 1, 2 or 3 heteroatoms independently selected from oxygen, nitrogen and sulfur, a 3, 4, 5 or 6 membered monocyclic heterocyclyl ring system comprising 1 or 2 heteroatoms independently selected from oxygen, nitrogen and sulfur and a 3, 4, 5 or 6 membered saturated or partially unsaturated carbocyclic ring system, wherein said (C₁-C₆)alkyl, phenyl, monocyclic heteroaryl ring, monocyclic heterocyclyl ring or carbocyclic ring system is optionally substituted with 1 or 2 substituents independently selected from halo, amino, methylamino, di(methyl)amino, nitro, hydroxy, methoxy, oxo, cyano, C(O)NH2, C(O)NHCH3, C(O)N(CH3)(CH3), CF3, OCF3, S(O)2CH3, S(O)CH3, S(O)2NH2, S(O)2NHCH3, S(O)2N(CH3)2, NHS(O)2CH3 and N(CH3)S(O)2CH3;

each R8 is independently selected from (C₁-C₆)alkyl, CF3, phenyl, a 5 or 6 membered monocyclic heteroaryl ring system comprising 1, 2 or 3 heteroatoms independently selected from oxygen, nitrogen and sulfur, a 3, 4, 5 or 6 membered monocyclic heterocyclyl ring system comprising 1 or 2 heteroatoms independently selected from oxygen, nitrogen and sulfur and a 3, 4, 5 or 6 membered saturated or partially unsaturated carbocyclic ring system, wherein said (C₁-C₆)alkyl, phenyl, monocyclic heteroaryl ring, monocyclic heterocyclyl ring or carbocyclic ring system is optionally substituted with 1 or 2 substituents independently selected from halo, amino, methylamino, di(methyl)amino, nitro, hydroxy, methoxy, oxo, cyano, C(O)NH2, C(O)NHCH3, C(O)N(CH3)(CH3), CF3, OCF3, S(O)2CH3, S(O)CH3, S(O)2NH2, S(O)2NHCH3, S(O)2N(CH3)2, NHS(O)2CH3 and N(CH3)S(O)2CH3; or a pharmaceutically acceptable salt thereof.

In some embodiments, NLRP3 antagonists which make up part of this invention include examples listed in table 1, page 79 of WO18015445. In some embodiments, NLRP3 antagonists which make up part of this invention include exemplary compounds of claim 14 of WO18015445.

Embodiments of the invention include NLRP3 antagonists specifically defined in WO 2019/068772, for example the compounds described in claim 14, in combination with an anti-TNFα agent; preferably in combination with Infliximab, Etanercept, Certolizumab pegol, Golimumab or Adalimumab; more preferably in combination with Adalimumab.

In some embodiments, the NLRP3 antagonist is a compound described in International application publication WO19043610, incorporated by reference herein. For example, a compound of formula as defined in claim 1, i.e. a compound of formula (118A), or a pharmaceutically acceptable salt, solvate or prodrug thereof:

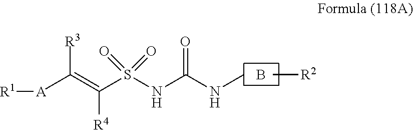

Wherein:

‘A’ is selected from unsubstituted or substituted (C₁-C₆)alkyl, (C₂-C₆)alkenyl, (C₃-C₇)cycloalkyl, aryl, heteroaryl, 4-7 membered heterocyclic ring system, 7- to 14-membered bicyclic heterocyclic ring system, bridged bicyclic heterocyclic or spiro cyclic system, having optionally one or more than one heteroatoms;

R1, which represents one or more substituents on ‘A’, at each occurrence independently represents hydrogen, halogen, haloalkyl, cyano, optionally substituted groups selected from (C₁-C₆)alkyl, (C₂-C₆)alkenyl, (C₁-C₆)alkoxy, (C₃-C₇)cycloalkyl, aryl, heteroaryl, heterocyclyl, benzyl, thiol, mercapto alkyl (sulfur and its oxidized forms, like S, SO₂), (C₁-C₆)thio-alkoxy groups;

In an embodiment when ‘A’ represents ring, R1 at each occurrence, may represent one or more substituents selected from hydrogen, halogen, haloalkyl, cyano, optionally substituted groups selected from (C1-C6)alkyl, (C1-C6)haloalkyl, (C2-C6)alkenyl, (C1-C6)alkoxy, (C3-C7)cycloalkyl, NH2, NH(C1-C6)alkyl, N(C3-C7)cycloalkyl; N(C1-C6 alkyl)2, aryl, heteroaryl, heterocyclyl, benzyl, thiol, mercapto alkyl, sulfur and its oxidized forms, (C1-C6)thio-alkoxy, bridged or spiro ring system having optionally one or more than one heteroatoms;

‘B’ is selected from optionally substituted (C₃-C₇)cycloalkyl, aryl, heteroaryl and heterocyclyl groups;

R2 at each occurrence independently represents hydrogen, halogen, cyano, optionally substituted groups selected from (C1-C6)alkyl, (C2-C6) alkenyl, (C1-C6)alkoxy (C3-C7)cycloalkyl, benzyl, aryl, heteroaryl, heterocyclyl, thiol, thioalkyl, sulfur and its oxidized forms, thio-alkoxy, bridged or spiro ring system having optionally one or more than one heteroatoms;

Each of R3, R4, R5, R6, R7, R8, R9 and R10 at each occurrence are independently selected from hydrogen, halogen, haloalkyl, cyano, nitro, amide, sulphonamide, acyl, hydroxyl, optionally substituted groups selected from (C1-C6)alkyl, (C1-C6)haloalkyl, (C3-C7)cycloalkyl, (C1-C6)alkoxy, thiol, mercapto alkyl, sulfur and its oxidized forms, benzyl, aryl, heteroaryl, heterocyclyl; Alternatively, R3 and R4 may form a bond;

Alternatively, R3 and ‘A’ together with the atom to which they are attached may form an optionally substituted 5 to 7 membered heterocyclic ring system having optionally one or more than one heteroatoms; Alternatively each of R5 and R6,

R7 and R8 or R8 and R9 wherever possible, together may form a 4 to 7 membered saturated or partially saturated ring containing from 0-2 additional heteroatoms selected from the group consisting of N, O, and S(O)p; p=1-2.

In some embodiments, NLRP3 antagonists which make up part of this invention include examples 1 to 78 of WO18015445. In some embodiments, NLRP3 antagonists which make up part of this invention include exemplary compounds of claim 12 of WO19043610.

Embodiments of the invention include NLRP3 antagonists specifically defined in WO19043610, for example the compounds described in claim 12, in combination with an anti-TNFα agent; preferably in combination with Infliximab, Etanercept, Certolizumab pegol, Golimumab or Adalimumab; more preferably in combination with Adalimumab.

In some embodiments, the NLRP3 antagonist is a compound described in International application publication WO17129897, incorporated by reference herein. For example, a compound of formula (119A) as defined in claim 1, i.e. a compound of formula (119A), or a pharmaceutically acceptable salt, solvate or prodrug thereof:

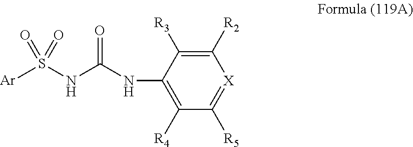

in which:
X represents a group C-R1 or a nitrogen atom;
R1 represents a hydrogen atom or a halogen;
R2, R3, R4 and R5 independently represent a hydrogen atom or a C₁-C₆ alkyl, R2 and R3 taken together possibly forming a cyclopentyl with the carbon atoms of the phenyl to which they are bonded, and R4 and R5 taken together possibly forming a cyclopentyl with the carbon atoms of the phenyl to which they are bonded; and
Ar represents a group chosen from:

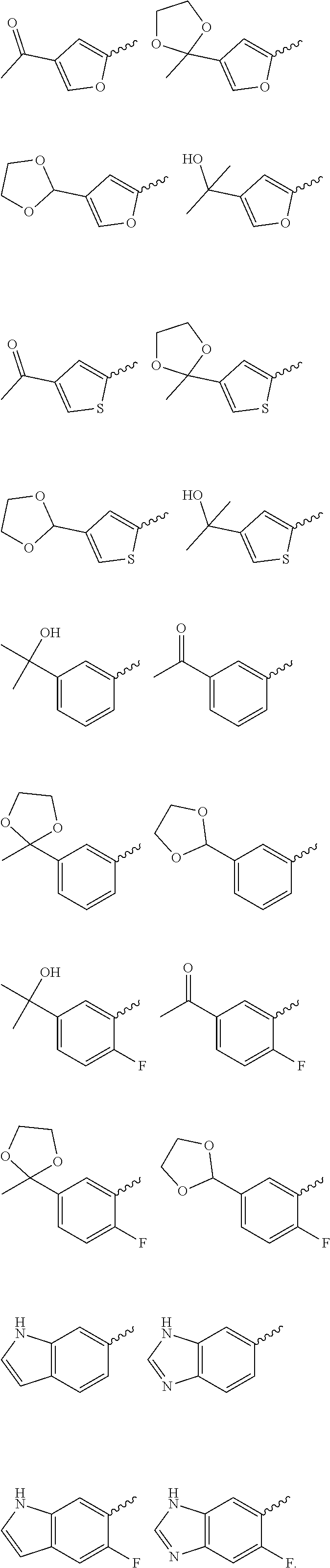

or a pharmaceutically acceptable salt, solvate or hydrate thereof.

In some embodiments, NLRP3 antagonists which make up part of this invention include examples 1 to 20 of WO17129897. In some embodiments, NLRP3 antagonists which make up part of this invention include exemplary compounds of claim 5 or 6 of WO17129897.

Embodiments of the invention include NLRP3 antagonists specifically defined in WO17129897, for example the compounds of examples 1 to 20 in combination with an anti-TNFα agent; preferably in combination with Infliximab, Etanercept, Certolizumab pegol, Golimumab or Adalimumab; more preferably in combination with Adalimumab. Preferably, a compound claim 5 of WO18136890 in combination with Adalimumab. More preferably, a compound claim 6 of WO18136890 in combination with Adalimumab.

In some embodiments, the NLRP3 antagonist is a compound described in International application publication WO 2019/121691, incorporated by reference herein. For example, a compound of formula (120A), having substituents as defined in claim 1, i.e. a compound of formula (120A), or a pharmaceutically acceptable salt, solvate or prodrug thereof:

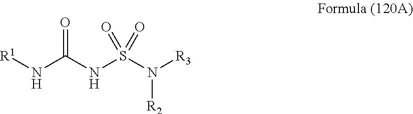

Wherein:

R1 is C3-C16 cycloalkyl, or C5-C10 aryl, wherein the C3-C8 monocyclic cycloalkyl, polycyclic cycloalkyl, or C5-C6 aryl is optionally substituted by one or more Ris; wherein each Ris is independently C1-C6 alkyl, C1-C6 haloalkyl, C1-C6 alkoxy, C1-C6 haloalkoxy, or halo;

R2 is —(CX2X2)n-R_{2S}, wherein n is 0, 1, or 2, and each X2 is independently H, C1-C6 alkyl, C2-C6 alkenyl, or C2-C6 alkynyl, wherein the C1-C6 alkyl, C2-C6 alkenyl, or C2-C6 alkynyl is optionally substituted with one or more halo, —CN, —OH, —O(Ci-C6 alkyl), —NH2, —NH(CI-C6 alkyl), —N(CI-C6 alkyl)2, or oxo;

R_{2S} is 4- to 8-membered heterocyclo alkyl optionally substituted with one or more C1-C6 alkyl, C2-C6 alkenyl, C2-C6 alkynyl, C1-C6 haloalkyl, halo, —CN, —OH, —O(Ci-C6 alkyl), —NH2, —NH(C1-C6 alkyl), —N(CI-C6 alkyl)2, or oxo; and

R3 is 7- to 12-membered heterocycloalkyl or 5- or 6-membered heteroaryl optionally substituted with one or more RJS, wherein each R3S is independently C1-C6 alkyl, C1-C6 haloalkyl, C3-C8 cycloalkyl, halo, or C3-C8 heterocycloalkyl wherein the C1-C6 alkyl, C1-C6 haloalkyl, C3-C8 cycloalkyl, or C3-C8 heterocycloalkyl is optionally substituted with —O(C1-C6 alkyl), —N(C1-C6 alkyl)2, halo, or —CN.

In some embodiments, NLRP3 antagonists which make up part of this invention include examples 1 to 20 of WO 2019/121691. In some embodiments, NLRP3 antagonists which make up part of this invention include the compounds of examples 1 to 116 of WO 2019/121691.

Embodiments of the invention include NLRP3 antagonists specifically defined in WO17129897, for example the compounds of examples 1 to 116 in combination with an anti-TNFα agent; preferably in combination with Infliximab, Etanercept, Certolizumab pegol, Golimumab or Adalimumab; more preferably in combination with Adalimumab.

### NLRP3 Inhibitory Nucleic Acids

In some embodiments of any of the methods described herein, the NLRP3 antagonist is an inhibitory nucleic acid. In some embodiments, the inhibitory nucleic acid is a short interfering RNA, an antisense nucleic acid, or a ribozyme.

Examples of aspects of these different oligonucleotides are described below. Any of the examples of inhibitory nucleic acids that are NLRP3 antagonists can decrease expression of NLRP3, ASC, CAP1, LCN-2, IL-18, IL-1β, S100A8, or S100A9 mRNA in a mammalian cell (e.g., a human cell). Any of the inhibitory nucleic acids described herein can be synthesized in vitro.

Inhibitory nucleic acids that can decrease the expression of NLRP3, ASC, CAP1, LCN-2, IL-18, IL-1β, S100A8, or S100A9 mRNA expression in a mammalian cell include antisense nucleic acid molecules, i.e., nucleic acid molecules whose nucleotide sequence is complementary to all or part of an NLRP3, ASC, CAP1, LCN-2, IL-18, IL-1(3, S100A8, or S100A9 mRNA (e.g., complementary to all or a part of any one of SEQ ID NOs: 38-57).

The nucleotides characterized by the Sequences ID NO: 73-92 are listed below and are being submitted in a separate and machine readable file.

Human NLRP3 CDS Transcript Variant 1 (SEQ ID NO: 73), Human NLRP3 CDS Transcript Variant 2 (SEQ ID NO: 74), Human NLRP3 CDS Transcript Variant 3 (SEQ ID NO: 75), Human NLRP3 CDS Transcript Variant 4 (SEQ ID NO: 76), Human NLRP3 CDS Transcript Variant 5 (SEQ ID NO: 77), Human NLRP3 CDS Transcript Variant 6 (SEQ ID NO: 78), Human ASC CDS Transcript Variant 1 (SEQ ID NO: 79), Human ASC CDS Transcript Variant 2 (SEQ ID NO: 80), Human CAP1 CDS Transcript Variant 1 (SEQ ID NO: 81), Human CAP1 CDS Transcript Variant 2 (SEQ ID NO: 82), Human IL-18 CDS Transcript Variant 1 (SEQ ID NO: 83), Human IL-18 CDS Transcript Variant 2 (SEQ ID NO: 84), Human IL-1β CDS Transcript (SEQ ID NO: 85), uman lipocalin-2 (LCN2) CDS Transcript (SEQ ID NO: 86), Human S100A8 CDS Transcript Variant 1 (SEQ ID NO: 87), Human S100A8 CDS Transcript Variant 2 (SEQ ID NO: 88), Human S100A8 CDS Transcript Variant 3 (SEQ ID NO: 89), Human S100A8 CDS Transcript Variant 4 (SEQ ID NO: 90), Human S100A8 CDS Transcript Variant 5 (SEQ ID NO: 91), and Human S100A9 CDS Transcript (SEQ ID NO: 92).

An antisense nucleic acid molecule can be complementary to all or part of a non-coding region of the coding strand of a nucleotide sequence encoding a NLRP3, ASC, CAP1, IL-18, IL-1β, LCN-2, S100A8, or S100A9 protein. Non-coding regions (5′ and 3′ untranslated regions) are the 5′ and 3′ sequences that flank the coding region in a gene and are not translated into amino acids.

Based upon the sequences disclosed herein, one of skill in the art can easily choose and synthesize any of a number of appropriate antisense nucleic acids to target a nucleic acid encoding a NLRP3, ASC, CAP1, IL-18, IL-1β, LCN-2, S100A8, or S100A9 protein described herein. Antisense nucleic acids targeting a nucleic acid encoding a NLRP3, ASC, CAP1, IL-18, IL-1β, LCN-2, S100A8, or S100A9 protein can be designed using the software available at the Integrated DNA Technologies website.

Examples of modified nucleotides which can be used to generate an antisense nucleic acid include 1-methylguanine, 1-methylinosine, 2,2-dimethylguanine, 2-methyladenine, 2-methylguanine, 3-methylcytosine, 2-methylthio-N6-isopentenyladenine, uracil-5-oxyacetic acid (v), wybutoxosine, pseudouracil, queosine, 2-thiocytosine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules described herein can be prepared in vitro and administered to a subject, e.g., a human subject. Alternatively, they can be generated in situ such that they hybridize with or bind to cellular mRNA and/or genomic DNA encoding a NLRP3, ASC, or CAP1 protein to thereby inhibit expression, e.g., by inhibiting transcription and/or translation. The hybridization can be by conventional nucleotide complementarities to form a stable duplex, or, for example, in the case of an antisense nucleic acid molecule that binds to DNA duplexes, through specific interactions in the major groove of the double helix. The antisense nucleic acid molecules can be delivered to a mammalian cell using a vector (e.g., an adenovirus vector, a lentivirus, or a retrovirus).

An antisense nucleic acid can be an α-anomeric nucleic acid molecule. An α-anomeric nucleic acid molecule forms specific double-stranded hybrids with complementary RNA in which, contrary to the usual, (3-units, the strands run parallel to each other (Gaultier et al., Nucleic Acids Res. 15:6625-6641, 1987). The antisense nucleic acid can also comprise a chimeric RNA-DNA analog (Inoue et al., FEBS Lett. 215:327-330, 1987) or a 2′-O-methylribonucleotide (Inoue et al., Nucleic Acids Res. 15:6131-6148, 1987).

Another example of an inhibitory nucleic acid is a ribozyme that has specificity for a nucleic acid encoding a NLRP3, ASC, CAP1, LCN-2, IL-18, IL-1β, S100A8, or S100A9 mRNA, e.g., specificity for any one of SEQ ID NOs: 38-57). Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature 334:585-591, 1988)) can be used to catalytically cleave mRNA transcripts to thereby inhibit translation of the protein encoded by the mRNA. NLRP3, ASC, CAP1, LCN-2, IL-18, IL-1β, S100A8, or S100A9 mRNA can be used to select a catalytic RNA having a specific ribonuclease activity from a pool of RNA molecules. See, e.g., Bartel et al., Science 261:1411-1418, 1993.

Alternatively, a ribozyme having specificity for a NLRP3, ASC, CAP1, LCN-2, IL-18, IL-1β, S100A8, or S100A9 mRNA sequences disclosed herein. For example, a derivative of a Tetrahymena L-19 IVS RNA can be constructed in which the nucleotide sequence of the active site is complementary to the nucleotide sequence to be cleaved in a NLRP3, ASC, CAP1, LCN-2, IL-18, IL-1β, S100A8, or S100A9 mRNA (see, e.g., U.S. Pat. Nos. 4,987,071 and 5,116,742).

An inhibitory nucleic acid can also be a nucleic acid molecule that forms triple helical structures. For example, expression of a NLRP3, ASC, CAP1, LCN-2, IL-18, IL-1β, S100A8, or S100A9 polypeptide can be inhibited by targeting nucleotide sequences complementary to the regulatory region of the gene encoding the NLRP3, ASC, CAP1, LCN-2, IL-18, IL-1β, S100A8, or S100A9 polypeptide (e.g., the promoter and/or enhancer, e.g., a sequence that is at least 1 kb, 2 kb, 3 kb, 4 kb, or 5 kb upstream of the transcription initiation start state) to form triple helical structures that prevent transcription of the gene in target cells. See generally Maher, Bioassays 14(12):807-15, 1992; Helene, Anticancer Drug Des. 6(6):569-84, 1991; and Helene, Ann. N.Y. Acad. Sci. 660:27-36, 1992.

In various embodiments, inhibitory nucleic acids can be modified at the sugar moiety, the base moiety, or phosphate backbone to improve, e.g., the solubility, stability, or hybridization, of the molecule. For example, the deoxyribose phosphate backbone of the nucleic acids can be modified to generate peptide nucleic acids (see, e.g., Hyrup et al., Bioorganic Medicinal Chem. 4(1):5-23, 1996). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to RNA and DNA under conditions of low ionic strength. PNA oligomers can be synthesized using standard solid phase peptide synthesis protocols (see, e.g., Perry-O'Keefe et al., Proc. Natl. Acad. Sci. U.S.A. 93:14670-675, 1996). PNAs can be used as antisense or antigene agents for sequence-specific modulation of gene expression by, e.g., inducing transcription or translation arrest or inhibiting replication.

### Pharmaceutical Compositions

In some embodiments, an NLRP3 antagonist (e.g., any of the NLRP3 antagonists described herein or known in the art) is administered as a pharmaceutical composition that includes the chemical entity and one or more pharmaceutically acceptable excipients, and optionally one or more additional therapeutic agents as described herein.

In some embodiments, an NLRP3 antagonist and/or an anti-TNFα agent is administered as a pharmaceutical composition that includes the NLRP3 antagonist and/or anti-TNFα agent and one or more pharmaceutically acceptable excipients, and optionally one or more additional therapeutic agents as described herein. Preferably the pharmaceutical composition includes an NLRP3 antagonist and an anti-TNFα agent. In some embodiments, the NLRP3 antagonist can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of the NLRP3 antagonists described herein. Dosage forms or compositions containing an NLRP3 antagonist and/or an anti-TNFα agent as described herein in the range of 0.005% to 100% with the balance made up from non-toxic excipient may be prepared. The contemplated compositions may contain 0.001%-100% of an NLRP3 antagonist and/or an anti-TNFα agent, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, the NLRP3 antagonist and/or the anti-TNFα agent (e.g., any of the exemplary NLRP3 antagonists or anti-TNFα agents described herein or known in the art) or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds (i.e. the NLRP3 antagonist and/or the anti-TNFα agent) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

In certain embodiments, the chemical entities described herein (i.e. the NLRP3 antagonist and/or the anti-TNFα agent) or a pharmaceutical composition thereof are suitable for local, topical administration to the digestive or GI tract, e.g., rectal administration. Rectal compositions include, without limitation, enemas, rectal gels, rectal foams, rectal aerosols, suppositories, jelly suppositories, and enemas (e.g., retention enemas).

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities (i.e. the NLRP3 antagonist and/or the anti-TNFα agent) with suitable nonirritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the NLRP3 antagonist and/or the anti-TNFα agent described herein, or a pharmaceutical composition thereof, are suitable for local delivery to the digestive or GI tract by way of oral administration (e.g., solid or liquid dosage forms).

Solid dosage forms for oral administration include capsules, tablets, pills, powders, and granules. In such solid dosage forms, the NLRP3 antagonist and/or the anti-TNFα agent is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with an NLRP3 antagonist and/or the anti-TNFα agent provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities (i.e. NLRP3 antagonists and/or the anti-TNFα agents) or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the NLRP3 antagonist and/or the anti-TNFα agent to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent (i.e. NLRP3 antagonist and/or the anti-TNFα agent) are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

Preferably the above pharmaceutical composition embodiments comprise an NLRP3 antagonist.

More preferably the above pharmaceutical composition embodiments comprise an NLRP3 antagonist and an anti-TNFα agent.

### Enema Formulations

In some embodiments, enema formulations containing any of the anti-TNFα agents and/or NLRP3 antagonists described herein are provided in “ready-to-use” form.

In some embodiments, enema formulations containing the chemical entities described herein are provided in one or more kits or packs. In certain embodiments, the kit or pack includes two or more separately contained/packaged components, e.g. two components, which when mixed together, provide the desired formulation (e.g., as a suspension). In certain of these embodiments, the two component system includes a first component and a second component, in which: (i) the first component (e.g., contained in a sachet) includes the NLPR3 antagonist and/or anti-TNFα agent (as described anywhere herein) and optionally one or more pharmaceutically acceptable excipients (e.g., together formulated as a solid preparation, e.g., together formulated as a wet granulated solid preparation); and (ii) the second component (e.g., contained in a vial or bottle) includes one or more liquids and optionally one or more other pharmaceutically acceptable excipients together forming a liquid carrier. Prior to use (e.g., immediately prior to use), the contents of (i) and (ii) are combined to form the desired enema formulation, e.g., as a suspension. In other embodiments, each of component (i) and (ii) is provided in its own separate kit or pack.

In some embodiments, each of the one or more liquids is water, or a physiologically acceptable solvent, or a mixture of water and one or more physiologically acceptable solvents. Typical such solvents include, without limitation, glycerol, ethylene glycol, propylene glycol, polyethylene glycol and polypropylene glycol. In certain embodiments, each of the one or more liquids is water. In other embodiments, each of the one or more liquids is an oil, e.g. natural and/or synthetic oils that are commonly used in pharmaceutical preparations.

Further pharmaceutical excipients and carriers that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe für Pharmazie, Kosmetik and angrenzende Gebiete (Edition Cantor Aulendorf, 1989)).

In some embodiments, each of the one or more pharmaceutically acceptable excipients can be independently selected from thickeners, viscosity enhancing agents, bulking agents, mucoadhesive agents, penetration enhancers, buffers, preservatives, diluents, binders, lubricants, glidants, disintegrants, fillers, solubilizing agents, pH modifying agents, preservatives, stabilizing agents, anti-oxidants, wetting or emulsifying agents, suspending agents, pigments, colorants, isotonic agents, chelating agents, emulsifiers, and diagnostic agents.

In certain embodiments, each of the one or more pharmaceutically acceptable excipients can be independently selected from thickeners, viscosity enhancing agents, mucoadhesive agents, buffers, preservatives, diluents, binders, lubricants, glidants, disintegrants, and fillers.

In certain embodiments, each of the one or more pharmaceutically acceptable excipients can be independently selected from thickeners, viscosity enhancing agents, bulking agents, mucoadhesive agents, buffers, preservatives, and fillers.

In certain embodiments, each of the one or more pharmaceutically acceptable excipients can be independently selected from diluents, binders, lubricants, glidants, and disintegrants.

Examples of thickeners, viscosity enhancing agents, and mucoadhesive agents include without limitation: gums, e.g. xanthan gum, guar gum, locust bean gum, tragacanth gums, karaya gum, ghatti gum, cholla gum, psyllium seed gum and gum arabic; poly(carboxylic acid-containing) based polymers, such as poly (acrylic, maleic, itaconic, citraconic, hydroxyethyl methacrylic or methacrylic) acid which have strong hydrogen-bonding groups, or derivatives thereof such as salts and esters; cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof; clays such as manomorillonite clays, e.g. Veegun, attapulgite clay; polysaccharides such as dextran, pectin, amylopectin, agar, mannan or polygalactonic acid or starches such as hydroxypropyl starch or carboxymethyl starch; polypeptides such as casein, gluten, gelatin, fibrin glue; chitosan, e.g. lactate or glutamate or carboxymethyl chitin; glycosaminoglycans such as hyaluronic acid; metals or water soluble salts of alginic acid such as sodium alginate or magnesium alginate; schleroglucan; adhesives containing bismuth oxide or aluminium oxide; atherocollagen; polyvinyl polymers such as carboxyvinyl polymers; polyvinylpyrrolidone (povidone); polyvinyl alcohol; polyvinyl acetates, polyvinylmethyl ethers, polyvinyl chlorides, polyvinylidenes, and/or the like; polycarboxylated vinyl polymers such as polyacrylic acid as mentioned above; polysiloxanes; polyethers; polyethylene oxides and glycols; polyalkoxys and polyacrylamides and derivatives and salts thereof. Preferred examples can include cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof (e.g., methyl cellulose); and polyvinyl polymers such as polyvinylpyrrolidone (povidone).

Examples of preservatives include without limitation: benzalkonium chloride, benzoxonium chloride, benzethonium chloride, cetrimide, sepazonium chloride, cetylpyridinium chloride, domiphen bromide (Bradosol®), thiomersal, phenylmercuric nitrate, phenylmercuric acetate, phenylmercuric borate, methylparaben, propylparaben, chlorobutanol, benzyl alcohol, phenyl ethyl alcohol, chlorohexidine, polyhexamethylene biguanide, sodium perborate, imidazolidinyl urea, sorbic acid, Purite®), Polyquart®), and sodium perborate tetrahydrate and the like.

In certain embodiments, the preservative is a paraben, or a pharmaceutically acceptable salt thereof. In some embodiments, the paraben is an alkyl substituted 4-hydroxybenzoate, or a pharmaceutically acceptable salt or ester thereof. In certain embodiments, the alkyl is a C₁-C₄ alkyl. In certain embodiments, the preservative is methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof.

Examples of buffers include without limitation: phosphate buffer system (sodium dihydrogen phosphate dehydrate, disodium phosphate dodecahydrate, bibasic sodium phosphate, anhydrous monobasic sodium phosphate), bicarbonate buffer system, and bisulfate buffer system.

Examples of disintegrants include, without limitation: carmellose calcium, low substituted hydroxypropyl cellulose (L-HPC), carmellose, croscarmellose sodium, partially pregelatinized starch, dry starch, carboxymethyl starch sodium, crospovidone, polysorbate 80 (polyoxyethylenesorbitan oleate), starch, sodium starch glycolate, hydroxypropyl cellulose pregelatinized starch, clays, cellulose, alginine, gums or cross linked polymers, such as cross-linked PVP (Polyplasdone XL from GAF Chemical Corp). In certain embodiments, the disintegrant is crospovidone.

Examples of glidants and lubricants (aggregation inhibitors) include without limitation: talc, magnesium stearate, calcium stearate, colloidal silica, stearic acid, aqueous silicon dioxide, synthetic magnesium silicate, fine granulated silicon oxide, starch, sodium laurylsulfate, boric acid, magnesium oxide, waxes, hydrogenated oil, polyethylene glycol, sodium benzoate, stearic acid glycerol behenate, polyethylene glycol, and mineral oil. In certain embodiments, the glidant/lubricant is magnesium stearate, talc, and/or colloidal silica; e.g., magnesium stearate and/or talc.

Examples of diluents, also referred to as “fillers” or “bulking agents” include without limitation: dicalcium phosphate dihydrate, calcium sulfate, lactose (e.g., lactose monohydrate), sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. In certain embodiments, the diluent is lactose (e.g., lactose monohydrate).

Examples of binders include without limitation: starch, pregelatinized starch, gelatin, sugars (including sucrose, glucose, dxtrose, lactose and sorbitol), polyethylene glycol, waxes, natural and synthetic gums such as acacia tragacanth, sodium alginate cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (povidone). In certain embodiments, the binder is polyvinylpyrrolidone (povidone).

In some embodiments, enema formulations containing the chemical entities described herein include water and one or more (e.g., all) of the following excipients:

One or more (e.g., one, two, or three) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);

One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof;

One or more (e.g., one or two; e.g., two) buffers, such as phosphate buffer system (e.g., sodium dihydrogen phosphate dehydrate, disodium phosphate dodecahydrate);

One or more (e.g., one or two, e.g., two) glidants and/or lubricants, such as magnesium stearate and/or talc;

One or more (e.g., one or two; e.g., one) disintegrants, such as crospovidone; and

One or more (e.g., one or two; e.g., one) diluents, such as lactose (e.g., lactose monohydrate).

In certain of these embodiments, the NLRP3 antagonist is a compound of any one of Formulas I-XII or a compound shown in any one of Tables 1-18, or a pharmaceutically acceptable salt and/or hydrate and/or cocrystal thereof.

In certain embodiments, enema formulations containing an NLRP3 antagonist and/or anti-TNFα agent described herein include water, methyl cellulose, povidone, methylparaben, propylparaben, sodium dihydrogen phosphate dehydrate, disodium phosphate dodecahydrate, crospovidone, lactose monohydrate, magnesium stearate, and talc. In certain of these embodiments, the NLRP3 antagonist is a compound of any one of Formulas I-XII or a compound shown in any one of Tables 1-18, or a pharmaceutically acceptable salt and/or hydrate and/or cocrystal thereof.

In certain embodiments, enema formulations containing an NLRP3 antagonist and/or anti-TNFα agent described herein are provided in one or more kits or packs. In certain embodiments, the kit or pack includes two separately contained/packaged components, which when mixed together, provide the desired formulation (e.g., as a suspension). In certain of these embodiments, the two component system includes a first component and a second component, in which: (i) the first component (e.g., contained in a sachet) includes the NLRP3 antagonist and/or anti-TNFα agent described herein (as described anywhere herein) and one or more pharmaceutically acceptable excipients (e.g., together formulated as a solid preparation, e.g., together formulated as a wet granulated solid preparation); and (ii) the second component (e.g., contained in a vial or bottle) includes one or more liquids and one or more one or more other pharmaceutically acceptable excipients together forming a liquid carrier. In other embodiments, each of component (i) and (ii) is provided in its own separate kit or pack.

In certain of these embodiments, component (i) includes the NLRP3 antagonist and/or anti-TNFα agent described herein (e.g., a compound of any one of Formulas I-XII or a compound shown in any one of Tables 1-18, or a pharmaceutically acceptable salt and/or hydrate and/or cocrystal thereof) and one or more (e.g., all) of the following excipients:

(a) One or more (e.g., one) binders (e.g., a polyvinyl polymer, such as polyvinylpyrrolidone (povidone);

(b) One or more (e.g., one or two, e.g., two) glidants and/or lubricants, such as magnesium stearate and/or talc;

(c) One or more (e.g., one or two; e.g., one) disintegrants, such as crospovidone; and

(d) One or more (e.g., one or two; e.g., one) diluents, such as lactose (e.g., lactose monohydrate).

In certain embodiments, component (i) includes from about 40 weight percent to about 80 weight percent (e.g., from about 50 weight percent to about 70 weight percent, from about 55 weight percent to about 70 weight percent; from about 60 weight percent to about 65 weight percent; e.g., about 62.1 weight percent) of the NLRP4 antagonist (e.g., a compound of any one of Formulas I-XII or a compound shown in any one of Tables 1-18, or a pharmaceutically acceptable salt and/or hydrate and/or cocrystal thereof) and/or anti-TNFα agent.

In certain embodiments, component (i) includes from about 0.5 weight percent to about 5 weight percent (e.g., from about 1.5 weight percent to about 4.5 weight percent, from about 2 weight percent to about 3.5 weight percent; e.g., about 2.76 weight percent) of the binder (e.g., povidone).

In certain embodiments, component (i) includes from about 0.5 weight percent to about 5 weight percent (e.g., from about 0.5 weight percent to about 3 weight percent, from about 1 weight percent to about 3 weight percent; about 2 weight percent e.g., about 1.9 weight percent) of the disintegrant (e.g., crospovidone).

In certain embodiments, component (i) includes from about 10 weight percent to about 50 weight percent (e.g., from about 20 weight percent to about 40 weight percent, from about 25 weight percent to about 35 weight percent; e.g., about 31.03 weight percent) of the diluent (e.g., lactose, e.g., lactose monohydrate).

In certain embodiments, component (i) includes from about 0.05 weight percent to about 5 weight percent (e.g., from about 0.05 weight percent to about 3 weight percent) of the glidants and/or lubricants.

In certain embodiments (e.g., when component (i) includes one or more lubricants, such as magnesium stearate), component (i) includes from about 0.05 weight percent to about 1 weight percent (e.g., from about 0.05 weight percent to about 1 weight percent; from about 0.1 weight percent to about 1 weight percent; from about 0.1 weight percent to about 0.5 weight percent; e.g., about 0.27 weight percent) of the lubricant (e.g., magnesium stearate).

In certain embodiments (when component (i) includes one or more lubricants, such as talc), component (i) includes from about 0.5 weight percent to about 5 weight percent (e.g., from about 0.5 weight percent to about 3 weight percent, from about 1 weight percent to about 3 weight percent; from about 1.5 weight percent to about 2.5 weight percent; from about 1.8 weight percent to about 2.2 weight percent; about 1.93 weight percent) of the lubricant (e.g., talc).

In certain of these embodiments, each of (a), (b), (c), and (d) above is present.

Preferably the above Enema formulation embodiments comprise an NLRP3 antagonist.

More preferably the above Enema formulation embodiments comprise an NLRP3 antagonist and an anti-TNFα agent.

In certain embodiments, component (i) includes the ingredients and amounts as shown in Table A.

TABLE A

| Ingredient | Weight Percent |
|---|---|
| A compound of any one of | 40 weight percent to about 80 weight percent (e.g., |
| Formulas I-XII or a compound | from about 50 weight percent to about 70 weight |
| shown in any one of Tables 1- | percent, from about 55 weight percent to about 70 |
| 18 | weight percent; from about 60 weight percent to |
| | about 65 weight percent; e.g., about 62.1 weight |
| | percent) |
| Crospovidone (Kollidon CL) | 0.5 weight percent to about 5 weight percent (e.g., |
| | from about 0.5 weight percent to about 3 weight |
| | percent, from about 1 weight percent to about 3 |
| | weight percent; about 1.93 weight percent |
| lactose monohydrate | about 10 weight percent to about 50 weight percent |
| (Pharmatose 200M) | (e.g., from about 20 weight percent to about 40 |
| | weight percent, from about 25 weight percent to |
| | about 35 weight percent; e.g., about 31.03 weight |
| | percent |
| Povidone (Kollidon K30) | about 0.5 weight percent to about 5 weight percent |
| | (e.g., from about 1.5 weight percent to about 4.5 |
| | weight percent, from about 2 weight percent to |
| | about 3.5 weight percent; e.g., about 2.76 weight |
| | percent |
| talc | 0.5 weight percent to about 5 weight percent (e.g., |
| | from about 0.5 weight percent to about 3 weight |
| | percent, from about 1 weight percent to about 3 |
| | weight percent; from about 1.5 weight percent to |
| | about 2.5 weight percent; from about 1.8 weight |
| | percent to about 2.2 weight percent;e.g., about 1.93 |
| | weight percent |
| Magnesium stearate | about 0.05 weight percent to about 1 weight percent |
| | (e.g., from about 0.05 weight percent to about 1 |
| | weight percent; from about 0.1 weight percent to |
| | about 1 weight percent; from about 0.1 weight |
| | percent to about 0.5 weight percent; e.g., about 0.27 |
| | weight percent |

In certain embodiments, component (1) includes the ingredients and amounts as shown in Table B.

TABLE B

| Ingredient | Weight Percent |
|---|---|
| A compound of any one of Formulas I- | About 62.1 weight percent) |
| XII or a compound shown in any one of | |
| Tables 1-18 | |
| Crospovidone (Kollidon CL) | About 1.93 weight percent |
| lactose monohydrate (Pharmatose 200M) | About 31.03 weight percent |
| Povidone (Kollidon K30) | About 2.76 weight percent |
| talc | About 1.93 weight percent |
| Magnesium stearate | About 0.27 weight percent |

In certain embodiments, component (i) is formulated as a wet granulated solid preparation. In certain of these embodiments an internal phase of ingredients (the NLRP3 antagonist, disintegrant, and diluent) are combined and mixed in a high-shear granulator. A binder (e.g., povidone) is dissolved in water to form a granulating solution. This solution is added to the Inner Phase mixture resulting in the development of granules. While not wishing to be bound by theory, granule development is believed to be facilitated by the interaction of the polymeric binder with the materials of the internal phase. Once the granulation is formed and dried, an external phase (e.g., one or more lubricants—not an intrinsic component of the dried granulation), is added to the dry granulation. It is believed that lubrication of the granulation is important to the flowability of the granulation, in particular for packaging.

In certain of the foregoing embodiments, component (ii) includes water and one or more (e.g., all) of the following excipients:

(a′) One or more (e.g., one, two; e.g., two) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);

(b′) One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof; and

(c′) One or more (e.g., one or two; e.g., two) buffers, such as phosphate buffer system (e.g., sodium dihydrogen phosphate dihydrate, disodium phosphate dodecahydrate);

In certain of the foregoing embodiments, component (ii) includes water and one or more (e.g., all) of the following excipients:

(a″) a first thickener, viscosity enhancing agent, binder, and/or mucoadhesive agent (e.g., a cellulose or cellulose ester or ether or derivative or salt thereof (e.g., methyl cellulose));

(a′″) a second thickener, viscosity enhancing agent, binder, and/or mucoadhesive agent (e.g., a polyvinyl polymer, such as polyvinylpyrrolidone (povidone));

(b″) a first preservative, such as a paraben, e.g., propyl 4-hydroxybenzoate (propylparaben), or a pharmaceutically acceptable salt or ester thereof;

(b″) a second preservative, such as a paraben, e.g., methyl 4-hydroxybenzoate (methylparaben), or a pharmaceutically acceptable salt or ester thereof,

(c″) a first buffer, such as phosphate buffer system (e.g., disodium phosphate dodecahydrate);

(c′″) a second buffer, such as phosphate buffer system (e.g., sodium dihydrogen phosphate dehydrate),

In certain embodiments, component (ii) includes from about 0.05 weight percent to about 5 weight percent (e.g., from about 0.05 weight percent to about 3 weight percent, from about 0.1 weight percent to about 3 weight percent; e.g., about 1.4 weight percent) of (a″).

In certain embodiments, component (ii) includes from about 0.05 weight percent to about 5 weight percent (e.g., from about 0.05 weight percent to about 3 weight percent, from about 0.1 weight percent to about 2 weight percent; e.g., about 1.0 weight percent) of (a′″).

In certain embodiments, component (ii) includes from about 0.005 weight percent to about 0.1 weight percent (e.g., from about 0.005 weight percent to about 0.05 weight percent; e.g., about 0.02 weight percent) of (b″).

In certain embodiments, component (ii) includes from about 0.05 weight percent to about 1 weight percent (e.g., from about 0.05 weight percent to about 0.5 weight percent; e.g., about 0.20 weight percent) of (b′″).

In certain embodiments, component (ii) includes from about 0.05 weight percent to about 1 weight percent (e.g., from about 0.05 weight percent to about 0.5 weight percent; e.g., about 0.15 weight percent) of (c″).

In certain embodiments, component (ii) includes from about 0.005 weight percent to about 0.5 weight percent (e.g., from about 0.005 weight percent to about 0.3 weight percent; e.g., about 0.15 weight percent) of (c′″).

In certain of these embodiments, each of (a″)-(c′) is present.

In certain embodiments, component (ii) includes water (up to 100%) and the ingredients and amounts as shown in Table C.

TABLE C

| Ingredient | Weight Percent |
|---|---|
| methyl cellulose (Methocel | 0.05 weight percent to about 5 weight percent (e.g., |
| A15C premium) | from about 0.05 weight percent to about 3 weight |
| | percent, from about 0.1 weight percent to about 3 |
| | weight percent; e.g., about 1.4 weight percent |
| Povidone (Kollidon K30) | 0.05 weight percent to about 5 weight percent (e.g., |
| | from about 0.05 weight percent to about 3 weight |
| | percent, from about 0.1 weight percent to about 2 |
| | weight percent; e.g., about 1.0 weight percent |
| propyl 4-hydroxybenzoate | about 0.005 weight percent to about 0.1 weight |
| | percent (e.g., from about 0.005 weight percent to |
| | about 0.05 weight percent; e.g., about 0.02 weight |
| | percent) |
| methyl 4-hydroxybenzoate | about 0.05 weight percent to about 1 weight percent |
| | (e.g., from about 0.05 weight percent to about 0.5 |
| | weight percent; e.g., about 0.20 weight percent) |
| disodium phosphate | about 0.05 weight percent to about 1 weight percent |
| dodecahydrate | (e.g., from about 0.05 weight percent to about 0.5 |
| | weight percent; e.g., about 0.15 weight percent) |
| sodium dihydrogen phospahate | about 0.005 weight percent to about 0.5 weight |
| dihydrate | percent (e.g., from about 0.005 weight percent to |
| | about 0.3 weight percent; e.g., about 0.15 weight |
| | percent) |

In certain embodiments, component (ii) includes water (up to 100%) and the ingredients and amounts as shown in Table D.

TABLE D

| Ingredient | Weight Percent |
|---|---|
| methyl cellulose (Methocel A15C | about 1.4 weight percent |
| premium) | |
| Povidone (Kollidon K30) | about 1.0 weight percent |
| propyl 4-hydroxybenzoate | about 0.02 weight percent |
| methyl 4-hydroxybenzoate | about 0.20 weight percent |
| disodium phosphate dodecahydrate | about 0.15 weight percent |
| sodium dihydrogen phospahate dihydrate | about 0.15 weight percent |

“Ready-to-use” enemas are generally be provided in a “single-use” sealed disposable container of plastic or glass. Those formed of a polymeric material preferably have sufficient flexibility for ease of use by an unassisted patient. Typical plastic containers can be made of polyethylene. These containers may comprise a tip for direct introduction into the rectum. Such containers may also comprise a tube between the container and the tip. The tip is preferably provided with a protective shield which is removed before use. Optionally the tip has a lubricant to improve patient compliance.

In some embodiments, the enema formulation (e.g., suspension) is poured into a bottle for delivery after it has been prepared in a separate container. In certain embodiments, the bottle is a plastic bottle (e.g., flexible to allow for delivery by squeezing the bottle), which can be a polyethylene bottle (e.g., white in color). In some embodiments, the bottle is a single chamber bottle, which contains the suspension or solution. In other embodiments, the bottle is a multichamber bottle, where each chamber contains a separate mixture or solution. In still other embodiments, the bottle can further include a tip or rectal cannula for direct introduction into the rectum. In some embodiments, the enema formulation can be delivered in the device that includes a plastic bottle, a breakable capsule, and a rectal cannula and single flow pack.

### Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the NLRP3 antagonists described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

In some embodiments, enema formulations include from about 0.5 mg to about 2500 mg (e.g., from about 0.5 mg to about 2000 mg, from about 0.5 mg to about 1000 mg, from about 0.5 mg to about 750 mg, from about 0.5 mg to about 600 mg, from about 0.5 mg to about 500 mg, from about 0.5 mg to about 400 mg, from about 0.5 mg to about 300 mg, from about 0.5 mg to about 200 mg; e.g., from about 5 mg to about 2500 mg, from about 5 mg to about 2000 mg, from about 5 mg to about 1000 mg; from about 5 mg to about 750 mg; from about 5 mg to about 600 mg; from about 5 mg to about 500 mg; from about 5 mg to about 400 mg; from about 5 mg to about 300 mg; from about 5 mg to about 200 mg; e.g., from about 50 mg to about 2000 mg, from about 50 mg to about 1000 mg, from about 50 mg to about 750 mg, from about 50 mg to about 600 mg, from about 50 mg to about 500 mg, from about 50 mg to about 400 mg, from about 50 mg to about 300 mg, from about 50 mg to about 200 mg; e.g., from about 100 mg to about 2500 mg, from about 100 mg to about 2000 mg, from about 100 mg to about 1000 mg, from about 100 mg to about 750 mg, from about 100 mg to about 700 mg, from about 100 mg to about 600 mg, from about 100 mg to about 500 mg, from about 100 mg to about 400 mg, from about 100 mg to about 300 mg, from about 100 mg to about 200 mg; e.g., from about 150 mg to about 2500 mg, from about 150 mg to about 2000 mg, from about 150 mg to about 1000 mg, from about 150 mg to about 750 mg, from about 150 mg to about 700 mg, from about 150 mg to about 600 mg, from about 150 mg to about 500 mg, from about 150 mg to about 400 mg, from about 150 mg to about 300 mg, from about 150 mg to about 200 mg; e.g., from about 150 mg to about 500 mg; e.g., from about 300 mg to about 2500 mg, from about 300 mg to about 2000 mg, from about 300 mg to about 1000 mg, from about 300 mg to about 750 mg, from about 300 mg to about 700 mg, from about 300 mg to about 600 mg; e.g., from about 400 mg to about 2500 mg, from about 400 mg to about 2000 mg, from about 400 mg to about 1000 mg, from about 400 mg to about 750 mg, from about 400 mg to about 700 mg, from about 400 mg to about 600 from about 400 mg to about 500 mg; e.g., 150 mg or 450 mg) of the chemical entity in from about 1 mL to about 3000 mL (e.g., from about 1 mL to about 2000 mL, from about 1 mL to about 1000 mL, from about 1 mL to about 500 mL, from about 1 mL to about 250 mL, from about 1 mL to about 100 mL, from about 10 mL to about 1000 mL, from about 10 mL to about 500 mL, from about 10 mL to about 250 mL, from about 10 mL to about 100 mL, from about 30 mL to about 90 mL, from about 40 mL to about 80 mL; from about 50 mL to about 70 mL; e.g., about 1 mL, about 5 mL, about 10 mL, about 15 mL, about 20 mL, about 25 mL, about 30 mL, about 35 mL, about 40 mL, about 45 mL, about 50 mL, about 55 mL, about 60 mL, about 65 mL, about 70 mL, about 75 mL, about 100 mL, about 250 mL, or about 500 mL, or about 1000 mL, or about 2000 mL, or about 3000 mL; e.g., 60 mL) of liquid carrier.

In certain embodiments, enema formulations include from about 50 mg to about 250 mg (e.g., from about 100 mg to about 200; e.g., about 150 mg) of the NLRP3 antagonist and/or anti-TNFα agent in from about 10 mL to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 150 mg of the NLRP3 antagonist and/or anti-TNFα agent in about 60 mL of the liquid carrier. In certain of these embodiments, the NLRP3 antagonist is a compound of any one of Formulas I-XII or a compound shown in any one of Tables 1-18, or a pharmaceutically acceptable salt and/or hydrate and/or cocrystal thereof. For example, enema formulations can include about 150 mg of a compound of any one of Formulas I-XII or a compound shown in any one of Tables 1-18 in about 60 mL of the liquid carrier.

In certain embodiments, enema formulations include from about 350 mg to about 550 mg (e.g., from about 400 mg to about 500; e.g., about 450 mg) of the and/or anti-TNFα agent in from about 10 mL to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 450 mg of the NLRP3 antagonist and/or anti-TNFα agent in about 60 mL of the liquid carrier. In certain of these embodiments, the NLRP3 antagonist and/or anti-TNFα agent is a compound of any one of Formulas I-XII or a compound shown in any one of Tables 1-18, or a pharmaceutically acceptable salt and/or hydrate and/or cocrystal thereof. For example, enema formulations can include about 450 mg of a compound of any one of Formulas I-XII or a compound shown in any one of Tables 1-18 in about 60 mL of the liquid carrier.

In some embodiments, enema formulations include from about from about 0.01 mg/mL to about 50 mg/mL (e.g., from about 0.01 mg/mL to about 25 mg/mL; from about 0.01 mg/mL to about 10 mg/mL; from about 0.01 mg/mL to about 5 mg/mL; from about 0.1 mg/mL to about 50 mg/mL; from about 0.01 mg/mL to about 25 mg/mL; from about 0.1 mg/mL to about 10 mg/mL; from about 0.1 mg/mL to about 5 mg/mL; from about 1 mg/mL to about 10 mg/mL; from about 1 mg/mL to about 5 mg/mL; from about 5 mg/mL to about 10 mg/mL; e.g., about 2.5 mg/mL or about 7.5 mg/mL) of the NLRP3 antagonist in liquid carrier. In certain of these embodiments, the NLRP3 antagonist and/or anti-TNFα agent is a compound of any one of Formulas I-XII or a compound shown in any one of Tables 1-18, or a pharmaceutically acceptable salt and/or hydrate and/or cocrystal thereof. For example, enema formulations can include about 2.5 mg/mL or about 7.5 mg/mL of a compound of any one of Formulas I-XII or a compound shown in any one of Tables 1-18 in liquid carrier.

### Regimens

### Kits

Also provided herein are kits containing one or more (e.g., at least 1, 2, 3, 4, 5, 6, 7, 8, 9, 10, 11, 12, 14, 15, 16, 18, or 20) of any of the pharmaceutical compositions described herein. In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein. The kits described herein are not so limited; other variations will be apparent to one of ordinary skill in the art.

### EXAMPLE

### Example 1. Study

The CARD8 gene is located within the inflammatory bowel disease (IBD) 6 linkage region on chromosome 19. CARD8 interacts with NLRP3, and Apoptosis-associated Speck-like protein to form a caspase-1 activating complex termed the NLRP3 inflammasome. The NLRP3 inflammasome mediates the production and secretion of interleukin-14, by processing pro-IL-14 into mature secreted IL-14. In addition to its role in the inflammasome, CARD8 is also a potent inhibitor of nuclear factor NF-κB. NF-κB activation is essential for the production of pro-IL-14. Since over-production of IL-14 and dyregulation of NF-κB are hallmarks of Crohn's disease, CARD8 is herein considered to be a risk gene for inflammatory bowel disease. A significant association of CARD8 with Crohn's disease was detected in two British studies with a risk effect for the minor allele of the non-synonymous single-nucleotide polymorphism (SNP) of a C allele at rs2043211. This SNP introduces a premature stop codon, resulting in the expression of a severely truncated protein. This variant CARD8 protein is unable to suppress NF-κB activity, leading to constitutive production of pro-IL-14, which is a substrate for the NLRP3 inflammasome. It is believed that a gain-of-function mutation in an NLRP3 gene (e.g., any of the gain-of-function mutations described herein, e.g., any of the gain-of-function mutations in an NLRP3 gene described herein) combined with a loss-of-function mutation in a CARD8 gene (e.g., a C allele at r52043211) results in the development of diseases related to increased NLRP3 inflammasome expression and/or activity. Patients having, e.g., a gain-of-function mutation in an NLRP3 gene and/or a loss-of-function mutation in a CARD8 gene are predicted to show improved therapeutic response to treatment with an NLRP3 antagonist.

A study is designed to determine: whether NLRP3 antagonists inhibit inflammasome function and inflammatory activity in cells and biopsy specimens from patients with Crohn's disease or ulcerative colitis; and whether the specific genetic variants identify patients with Crohn's disease or ulcerative colitis who are most likely to respond to treatment with an NLRP3 antagonist.

The secondary objectives of this study are to: determine if an NLRP3 antagonist reduces inflammasome activity in Crohn's disease and ulcerative biopsy samples (comparing Crohn's disease and ulcerative colitis results with control patient results); determine if an NLRP3 antagonist reduced inflammatory cytokine RNA and protein expression in Crohn's disease and ulcerative colitis samples; determine if baseline (no ex vivo treatment) RNA levels of NLRP3, ASC, and IL-14 are greater in biopsy samples from patients with anti-TNFα agent resistance status; and stratify the results according to presence of specific genetic mutations in genes encoding ATG16L1, NLRP3, and CARD8 (e.g., any of the mutations in the ATG16L1 gene, NLRP3 gene, and CARD8 gene described herein).

### Methods

Evaluation of baseline expression of NLRP3 RNA and quantify inhibition of inflammasome activity by an NLRP3 antagonist in biopsies of disease tissue from patients with Crohn's disease and ulcerative colitis.
Determine if NLRP3 antagonist treatment reduces the inflammatory response in biopsies of disease from patients with Crohn's disease based on decreased expression of inflammatory gene RNA measured with Nanostring.
Sequence patient DNA to detect specific genetic mutations in the ATG16L1 gene, NLRP3 gene, and CARD8 gene (e.g., any of the exemplary mutations in these genes described herein) and then stratify the results of functional assays according to the presence of these genetic mutations.

### Experimental Design

Human subjects and tissue:
Endoscopic or surgical biopsies from areas of disease in patients with Crohn's disease and ulcerative colitis who are either anti-TNFα treatment naïve or resistant to anti-TNFα treatment; additionally biopsies from control patients (surveillance colonoscopy or inflammation-free areas from patients with colorectal cancer) are studied.

Ex vivo Treatment Model:
Organ or LPMC culture as determined appropriate

Endpoints to be measured:
Before ex vivo treatment—NLRP3 RNA level
After ex vivo treatment—inflammasome activity (either processed IL-14, processed caspase-1, or secreted I1-14); RNA for inflammatory cytokines (Nanostring); viable T cell number and/or T cell apoptosis.

Data Analysis Plan:
Determine if NLRP3 antagonist treatment decreases processed IL-14, processed caspase-1 or secreted I1-14, and inflammatory cytokine RNA levels.
Stratify response data according to treatment status at biopsy and the presence of genetic mutations in the NLRP3 gene, CARD8 gene, and ATG16L1 gene (e.g., any of the exemplary genetic mutations of these genes described herein).

### Example 2. Treatment of Anti-TNFα Resistant Patients with NLRP3 Antagonists

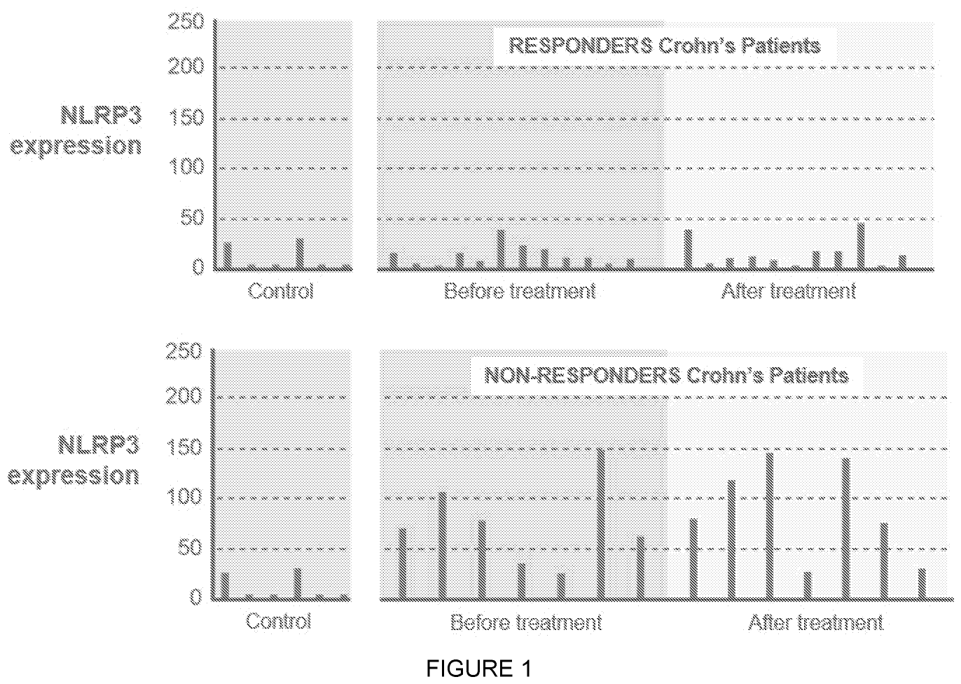

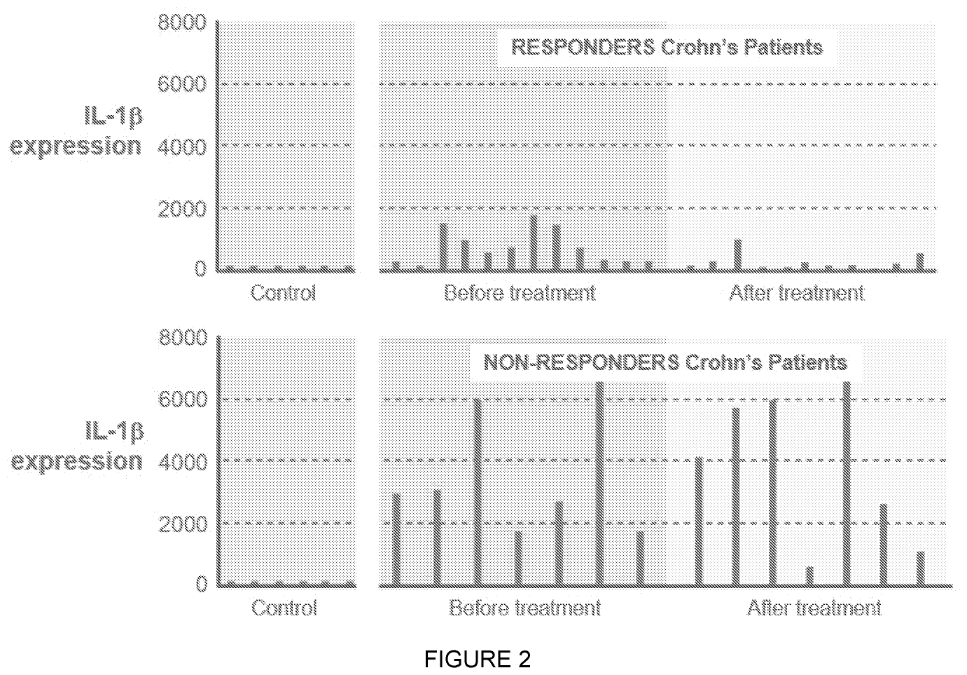

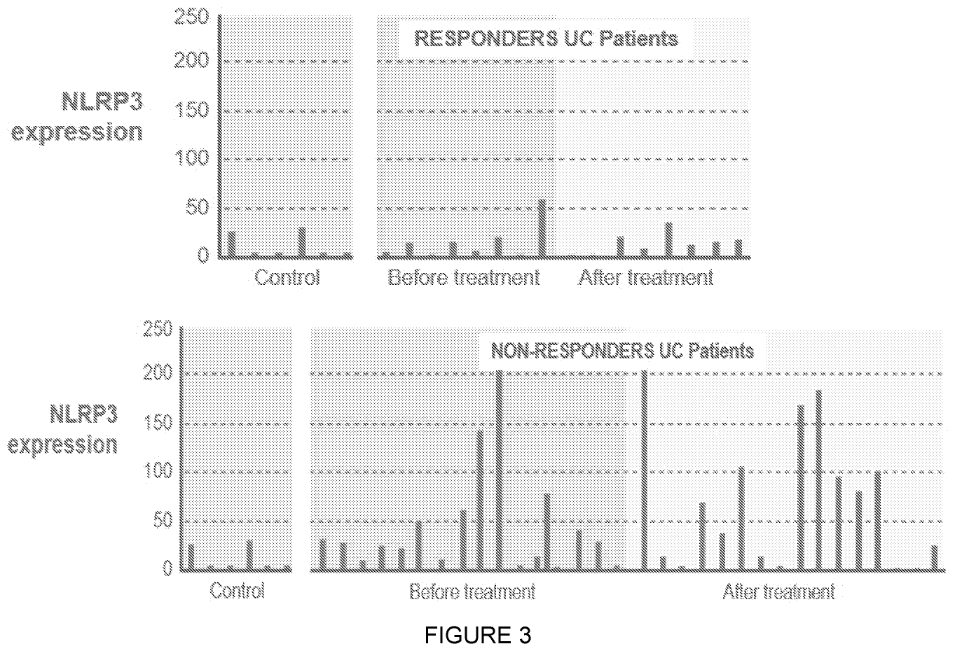

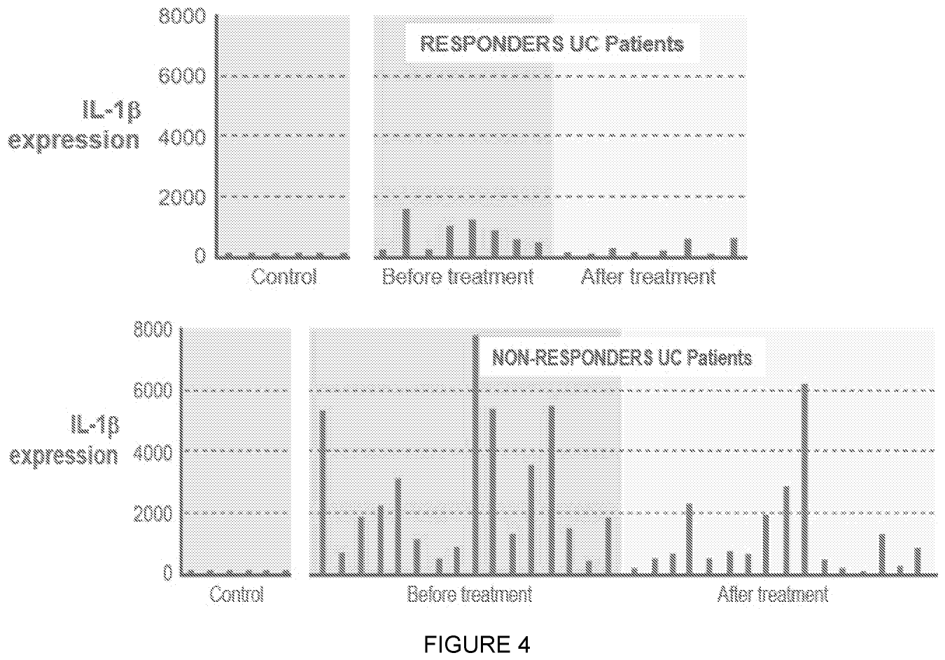

PLoS One 2009 Nov. 24; 4(11):e7984, describes that mucosal biopsies were obtained at endoscopy in actively inflamed mucosa from patients with Ulcerative Colitis, refractory to corticosteroids and/or immunosuppression, before and 4-6 weeks after their first infliximab (an anti-TNFα agent) infusion and in normal mucosa from control patients. The patients in this study were classified for response to infliximab based on endoscopic and histologic findings at 4-6 weeks after first infliximab treatment as responder or non-responder. Transcriptomic RNA expression levels of these biopsies were accessed by the inventors of the invention disclosed herein from GSE 16879, the publically available Gene Expression Omnibus (https://www.ncbi.nlm.nih.gov/geo/geo2r/?acc=GSE16879). Expression levels of RNA encoding NLRP3 and IL-1β were determined using GEO2R (a tool available on the same website), based on probe sets 207075 at and 205067 at, respectively. It was surprisingly found that in Crohn's disease patients that are non-responsive to the infliximab (an anti-TNFα agent) have higher expression of NLRP3 and IL-10 RNA than responsive patients (FIGS. 1 and 2). Similar surprising results of higher expression of NLRP3 and IL-1β RNA in UC patients that are non-responsive to infliximab (an anti-TNFα agent) compared to infliximab (an anti-TNFα agent) responsive patients (FIGS. 3 and 4) were found.

Said higher levels of NLRP3 and IL113 RNA expression levels in anti-TNFα agent non-responders, is hypothesised herein to lead to NLRP3 activation which in turns leads of release of IL-1β that induces IL-23 production, leading to said resistance to anti-TNFα agents. Therefore, treatment of Crohn's and UC anti-TNFα non-responders with an NLRP3 antagonist would prevent this cascade, and thus prevent development of non-responsiveness to anti-TNFα agents. Indeed, resistance to anti-TNFα agents is common in other inflammatory or autoimmune diseases. Therefore, use of an NLRP3 antagonist for the treatment of inflammatory or autoimmune diseases will block the mechanism leading to non-responsiveness to anti-TNFα□ agents. Consequently, use of NLRP3 antagonists will increase the sensitivity of patients with inflammatory or autoimmune diseases to anti-TNFα agents, resulting in a reduced dose of anti-TNFα agents for the treatment of these diseases. Therefore, a combination of an NLRP3 antagonist and an anti-TNFα agent can be used in the treatment of diseases wherein TNFα is overexpressed, such as inflammatory or autoimmune diseases, to avoid such non-responsive development of patients to anti-TNFα agents. Preferably, this combination treatment can be used in the treatment of IBD, for example Crohn's disease and UC.

Further, use of NLRP3 antagonists offers an alternative to anti-TNFα agents for the treatment of diseases wherein TNFα is overexpressed. Therefore, NLRP3 antagonists offers an alternative to anti-TNFα agents inflammatory or autoimmune diseases, such as IBD (e.g. Crohn's disease and UC).

Systematic anti-TNFα agents are also known to increase the risk of infection. Gut restricted NLRP3 antagonists, however, offers a gut targeted treatment (i.e. non-systemic treatment), preventing such infections. Therefore, treatment of TNFα gut diseases, such as IBD (i.e. Crohn's disease and UC), with gut restricted NLRP3 antagonists has the additional advantage of reducing the risk of infection compared to anti-TNFα agents.

Proposed Experiment:

Determine the expression of NLRP3 and caspase-1 in LPMCs and epithelial cells in patients with non-active disease, in patients with active disease, in patients with active disease resistant to corticosteroids, patients with active disease resistant to TNF-blocking agents. The expression of NLRP3 and caspase-1 in LPMCs and epithelial cells will be analyzed by RNAScope technology. The expression of active NLRP3 signature genes will be analyzed by Nanostring technology. A pilot analysis to determine feasibility will be performed with 5 samples from control, 5 samples from active CD lesions, and 5 samples from active UC lesions.

### Example 4. Study

It is presented that NLRP3 antagonists reverse resistance to anti-TNF induced T cell depletion/apoptosis in biopsy samples from IBD patients whose disease is clinically considered resistant or unresponsive to anti-TNF therapy.

A study is designed to determine: whether NLRP3 antagonists inhibit inflammasome function and inflammatory activity in cells and biopsy specimens from patients with Crohn's disease or ulcerative colitis; and whether an NLRP3 antagonist will synergize with anti-TNFα therapy in patients with Crohn's disease or ulcerative colitis.

The secondary objectives of this study are to: determine if an NLRP3 antagonist reduces inflammasome activity in Crohn's disease and ulcerative biopsy samples (comparing Crohn's disease and ulcerative colitis results with control patient results); determine if an NLRP3 antagonist reduced inflammatory cytokine RNA and protein expression in Crohn's disease and ulcerative colitis samples; determine if an NLRP3 antagonist in the absence of co-treatment with anti-TNFα antibody induces T cell depletion in Crohn's disease and ulcerative colitis biopsy samples; and determine if baseline (no ex vivo treatment) RNA levels of NLRP3, ASC, and IL-1β are greater in biopsy samples from patients with anti-TNFα agent resistance status.

### Methods

Evaluation of baseline expression of NLRP3 RNA and quantify inhibition of inflammasome activity by an NLRP3 antagonist in biopsies of disease tissue from patients with Crohn's disease and ulcerative colitis.
Determine if there is synergy between an NLRP3 antagonist and anti-TNF antibody with respect to effects on T cell depletion/apoptosis in biopsies of disease from patients with Crohn's disease and ulcerative colitis.
Determine if NLRP3 antagonist treatment reduces the inflammatory response in biopsies of disease from patients with Crohn's disease based on decreased expression of inflammatory gene RNA measured with Nanostring.

### Experimental Design

Human subjects and tissue:
Endoscopic or surgical biopsies from areas of disease in patients with Crohn's disease and ulcerative colitis who are either anti-TNFα treatment naïve or resistant to anti-TNFα treatment; additionally biopsies from control patients (surveillance colonoscopy or inflammation-free areas from patients with colorectal cancer) are studied.

Ex vivo Treatment Model:
Organ or LPMC culture as determined appropriate

Ex vivo Treatments:
NLRP3 antagonist (2 concentrations), negative control (vehicle), positive control (caspase-1 inhibitor) each in the presence or absence of anti-TNF antibody at a concentration appropriate to distinguish differences in the T cell apoptotic between biopsies from anti-TNF resistant and anti-TNF-sensitive Crohn's disease patients. Each treatment condition is evaluated in a minimum in duplicate samples.

Endpoints to be measured:
Before ex vivo treatment—NLRP3 RNA level
After ex vivo treatment—inflammasome activity (either processed IL-1β, processed caspase-1, or secreted I1-1β); RNA for inflammatory cytokines (Nanostring); viable T cell number and/or T cell apoptosis.

Data Analysis Plan:
Determine if NLRP3 antagonist co-treatment increases T cell apoptosis/deletion in response to anti-TNF.
Determine if the level of NLRP3 RNA expression is greater in TNF-resistant Crohn's disease and ulcerative colitis samples compared to anti-TNF treatment-naïve samples.
Determine if NLRP3 antagonist treatment decreases processed IL-1β, processed caspase-1 or secreted I1-1β, and inflammatory cytokine RNA levels.
   Biological Assay—Nigericin-stimulated IL-1b secretion assay in THP-1 cells

Monocytic THP-1 cells (ATCC: TIB-202) were maintained according to providers' instructions in RPMI media (RPMI/Hepes+10% fetal bovine serum+Sodium Pyruvate+0.05 mM Beta-mercaptoethanol (1000× stock)+Pen-Strep). Cells were differentiated in bulk with 0.5 μM phorbol 12-myristate 13-acetate (PMA; Sigma #P8139) for 3 hours, media was exchanged, and cells were plated at 50,000 cells per well in a 384-well flat-bottom cell culture plates (Greiner, #781986), and allowed to differentiate overnight. Compound in a 1:3.16 serial dilution series in DMSO was added 1:100 to the cells and incubated for 1 hour. The NLRP3 inflammasome was activated with the addition of 15 μM (final concentration) Nigericin (Enzo Life Sciences, #BML-CA421-0005), and cells were incubated for 3 hours. 10 μL supernatant was removed, and IL-1β levels were monitored using an HTRF assay (CisBio, #621L1PEC) according to manufacturers' instructions. Viability and pyroptosis was monitored with the addition of PrestoBlue cell viability reagent (Life Technologies, #A13261) directly to the cell culture plate.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An NLRP3 antagonist for use in the treatment or the prevention of a condition mediated by TNF-α, in a patient in need thereof, wherein the NLRP3 antagonist is administered to said patient at a therapeutically effective amount.

2. An NLRP3 antagonist for use according to claim 1, wherein the subject is resistant to treatment with an anti-TNFα agent.

3. An NLRP3 antagonist for use according to any preceeding claim, wherein the condition is a gut disease or disorder.

4. An NLRP3 antagonist for use according to any preceeding claim, wherein the NLRP3 antagonist is a gut-targeted NLRP3 antagonist.

5. An NLRP3 antagonist for use according to any preceeding claim, wherein the condition is Inflammatory Bowel Disease.

6. An NLRP3 antagonist for use according to any preceeding claim, wherein the condition is Crohn's Disease, or Ulcerative Colitis.

7. A pharmaceutical composition comprising an NLRP3 antagonist and at least one pharmaceutically acceptable excipient, for use according to any preceding claim.

8. A pharmaceutical composition of claim 7, further comprising an anti-TNFα agent, preferably wherein the anti-TNFα agent is Infliximab, Etanercept, Certolizumab pegol, Golimumab or Adalimumab, more preferably wherein the anti-TNFα agent is Adalimumab.

9. A pharmaceutical combination of an NLRP3 antagonist and an anti-TNFα agent for use according to any one of claims 1 to 6, preferably wherein the anti-TNFα agent is Infliximab, Etanercept, Certolizumab pegol, Golimumab or Adalimumab, more preferably wherein the anti-TNFα agent is Adalimumab.

10. A method of treating a subject in need thereof, the method comprising:
a. identifying a subject having resistance to an anti-TNFα agent; and
b. administering a treatment comprising a therapeutically effective amount of an NLRP3 antagonist or a pharmaceutically acceptable salt, solvate, or co-crystal thereof to the identified subject.

11. A method of treating a subject in need thereof, the method comprising administering a treatment comprising a therapeutically effective amount of an NLRP3 antagonist or a pharmaceutically acceptable salt, solvate, or co-crystal thereof to a subject identified as having resistance to an anti-TNFα agent.

12. The method of claim 10, wherein step (b) further comprises identifying the subject as also having an elevated level of NLRP3 inflammasome activity and/or expression in a cell obtained from the subject, as compared to a reference level.

13. The method of claim 11, wherein the identified subject also has an elevated level of level of NLRP3 inflammasome activity and/or expression in a cell obtained from the subject, as compared to a reference level.

14. The method of claim 12 or 13, wherein the NLRP3 inflammasome activity is any one of: secretion of IL-18 or IL-1β; caspase-1 activity; lipocalin-2; S100A8; and S100A9.

14. The method of claim 12, wherein the identifying the subject as also having a cell that has an elevated level of NLRP3 inflammasome expression comprises detecting the level of one or more of NLRP3 protein, ASC protein, procaspase-1 protein, and caspase-1 protein.

15. The method of claim 12, wherein the identifying the subject as also having a cell that has an elevated level of NLRP3 inflammasome expression comprises detecting the level of one or more of NLRP3 mRNA, ASC mRNA, and procaspase-1 mRNA.

16. The method of claim 13, wherein the identified subject also having a cell that has an elevated level of NLRP3 inflammasome expression has been determined to have a cell having an elevated level of one or more of NLRP3 protein, ASC protein, procaspase-1 protein, and capsase-1 protein.

17. The method of claim 13, wherein the identified subject also having a cell that has an elevated level of NLRP3 inflammasome expression has been determined to have a cell having an elevated level of one or more of NLRP3 mRNA, ASC mRNA, and procaspase-1 mRNA.

18. The method of any one of claims 1-17, further comprises administering a therapeutically effective amount of an anti-TNFα agent, in addition to the NLRP3 antagonist.

19. A method of reducing the risk of developing resistance to an anti-TNFα agent in a subject in need thereof, the method comprising:
a. administering to a subject in need thereof a therapeutically effective amount of an anti-TNFα agent and a therapeutically effective amount of an NLRP3 antagonist or a pharmaceutically acceptable salt, solvate, or co-crystal thereof.

20. The method of claim 19, wherein the anti-TNFα agent and the NLRP3 antagonist or a pharmaceutically acceptable salt, solvate, or co-crystal thereof are administered at substantially the same time.

21. The method of claim 20, wherein the anti-TNFα agent and the NLRP3 antagonist or a pharmaceutically acceptable salt, solvate, or co-crystal thereof are formulated into a single dosage form.

22. The method of claim 21, wherein the NLRP3 antagonist or a pharmaceutically acceptable salt, solvate, or co-crystal thereof is administered to the subject prior to administration of the anti-TNFα agent.

23. The method of claim 22, wherein the anti-TNFα agent is administered to the subject prior to administration of the NLRP3 antagonist or a pharmaceutically acceptable salt, solvate, or co-crystal thereof.

24. A method of treating a subject in need thereof, the method comprising administering a treatment comprising (i) a therapeutically effective amount of an NLRP3 antagonist or a pharmaceutically acceptable salt, solvate, or co-crystal thereof, and (ii) a therapeutically effective amount of an anti-TNFα agent, to a subject identified as having an elevated level of NLRP3 inflammasome activity and/or expression in a cell obtained from the subject, as compared to a reference level.

25. The method of any one of claims 10 to 24, wherein the subject has been diagnosed or identified as having an inflammatory or an autoimmune disorder.

26. The method of claim 25, wherein the inflammatory or autoimmune disorder is selected from the group consisting of: sickle cell disease, rheumatoid arthritis, juvenile arthritis, psoriatic arthritis, plaque psoriasis, ankylosing spondylitis, ulcerative colitis, Crohn's disease, inflammatory bowel disease, Behcet's disease, Takayasu's arteritis, atherosclerosis, gout, psoriasis, an infectious disease, asthma, peptic ulcer, periodontitis, dermatitis, diverticulitis, fibromyalgia, hepatitis, systemic lupus erythematosus, nephritis, appendicitis, bursitis, cystitis, encephalitis, gingivitis, meningitis, myelitis, neuritis, pharyngitis, phlebitis, prostatitis, rhinitis, sinusitis, tendonitis, testiculitis, tonsillitis, urethritis, vasculitis, vaginitis, Celiac disease, diverticulitis, glomerulonephritis, hidradenitis suppurativa, hypersensitivities, interstitial cystitis, Lichen planus, mast cell activation syndrome, mastocystosis, otitis, pelvic inflammatory disease, reperfusion injury, rheumatic fever, rhinitis, sarcoidosis, graft versus host disease, vasculitis, allergy, cancer, HIV, AIDS, scleroderma, Sjøgren's syndrome, anti-phospholipid antibody syndrome, myocarditis, postmyocardial infarction syndrome, postpericardiotomy syndrome, subacute bacterial endocarditis, anti-glomerular basement membrane nephritis, interstitial cystitis, lupus nephritis, autoimmune nephritis, autoimmune hepatitis, autoimmune hepatitis, primary biliary cholangitis, primary sclerosing cholangitis, primary schlerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, Lichen sclerosus, linear IgA disease, morphea, pemphigus vulgaris, pityriasis lichenoides et varioliforms acuta, Mucha-Habermann disease, psoriasis, systemic scleroderma, vitiligo, Addison's disease, autoimmune polyendocrine syndrome type 1, 2, or 3, autoimmune pancreatitis, diabetes mellitus type 1, autoimmune thyroiditis, Ord's thyroiditis, Graves' disease, autoimmune oophoritis, endometriosis, autoimmune orchitis, Sjogren's syndrome, autoimmune enteropathy, microscopic colitis, antiphospholipid syndrome, aplastic anemia, autoimmune hemolytic anemia, autoimmune lymphoproliferative syndrome, autoimmune neutropenia, autoimmune thrombocytopenic purpura, cold agglutinin disease, essential mixed cyroglobulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, Lyme disease, mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, sarcoidosis, Schnitzler syndrome, undifferentiated connective tissue disease, dermatomyositis, fibromyalgia, inclusion body myositis, myasthenia gravis, neuromyotonia, paraneoplastic cerebellar degeneration, polymyositis, acute disseminated encephalomyelitis, acute motor axonal neuropathy, anti-N-methyl-D-aspartate receptor encephalitis, Balo concentric sclerosis, Bickerstaff's encephalitis, chronic inflamatory demyelinating polyneuropathy, Guillain-Barre syndrome, Hashimoto's encephalopathy, idiopathic inflammatory demyelinating disease, Lambert-Eaton myasthenic syndrome, multiple sclerosis, Oshtoran syndrome, pediatric autoimmune neuropsychiatric disorder associated with Streptococcus, progressive inflammatory neuropathy, restless leg syndrome, Stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, Opsoclonus myoclonus syndrome, optic neuritis, scleritis, Susac's syndrome, sympathetic ophthalmia, Tolosa-Hunt syndrome, autoimmune inner ear disease, Meniere's disease, Behcet's disease, eosinophilic graunulomatosis with polyangiitis, giant cell arteritis, graunulomatosis with polyangiitis, IgA vasculitis, Kawasaki's disease, leukocytoclastic vasculitis, lupus vasculitis, microscopic polyangiitis, polyarteritis nodosa, polymyalgia rheumatica, urticarial vasculitis, vasculitis, primary immune deficiency, chronic fatigue syndrome, complex regional pain syndrome, eosinophilic esophagitis, gastritis, interstitial lung disease, POEMS syndrome, Raynaud's phenomenon, primary immunodeficiency, and pyoderma gangrenosum.

27. The method of claim 25, wherein the inflammatory or autoimmune disorder is Crohn's disease or ulcerative colitis.

28. The method of claim 25, wherein the inflammatory or autoimmune disorder is inflammatory bowel syndrome.

29. The method of any one of claims 10-28, wherein the anti-TNFα agent is an antibody or an antigen-binding antibody fragment, or a soluble TNFα receptor.

30. The method of claim 29, wherein the antibody is selected from the group consisting of: adalimumab, certolizumab, etanercept, golimumab, infliximab, CDP571, and certolizumab pegol.
The method of any one of claims 10-28, wherein the anti-TNFα agent is any one of: a small molecule inhibitor of a signaling component downstream of a TNFα receptor; an inhibitory nucleic acid; or a fusion protein.

31. The method of any one of claims 10-30, wherein the NLRP3 antagonist is an inhibitory nucleic acid.

32. The method of claim 30, wherein the inhibitory nucleic acid is a short interfering RNA, an antisense nucleic acid, or a ribozyme.

33. The method of any one of claims 10-30, wherein the NLRP3 antagonist is a compound of any one of Formulas I-XII, or a pharmaceutically acceptable salt thereof.

34. The method of any one of claims 1-86, wherein the NLRP3 antagonist is a compound shown in any one of Tables 1-18, or a pharmaceutically acceptable salt thereof.

35. A method of treating a subject, the method comprising administering a therapeutically effective amount of an NLPR3 antagonist or a pharmaceutically acceptable salt, solvate, or co-crystal thereof to a subject identified as having a cell that has an elevated level of NLRP3 inflammasome activity and/or expression as compared to a reference level.

36. The method of any one of claim 35, wherein the identifying a subject having a cell that has an elevated level of NLRP3 inflammasome activity comprises detecting a gain-of-function mutation in an NLRP3 gene in a cell from the subject.

37. The method of any one of claim 35 or 36, wherein the identifying a subject having a cell that has an elevated level of NLRP3 inflammasome activity comprises detecting a loss-of-function mutation in a CARD8 gene in a cell from the subject.

38. The method of any one of claim 35, wherein the subject has or is suspected of having Crohn's disease, inflammatory bowel disease (IBD), or other gastrointestinal, autoimmune, or autoinflammatory disorders.

39. The method of any one of the preceding claims, wherein the NLRP3 antagonist is a compound of Formula VII 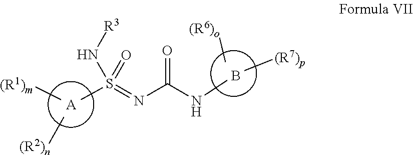
wherein
m=0, 1, or 2
n=0, 1, or 2
o=1 or 2
p=0, 1, 2, or 3
wherein
A is a 5- to 10-membered monocyclic or bicyclic heteroaryl or a C₆-C₁₀ monocyclic or bicyclic aryl;
B is a 5- to 10-membered monocyclic or bicyclic heteroaryl or a C₆-C₁₀ monocyclic or bicyclic aryl;
wherein
at least one R⁶ is ortho to the bond connecting the B ring to the NH(CO) group of Formula VII;
R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO—C₆-C₁₀ aryl; CO (5- to 10-membered heteroaryl); CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl and 3- to 7-membered heterocycloalkyl,
wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3-to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;
or at least one pair of R¹ and R² on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹ wherein the C₁-C₆ alkyl and C₁-C₆ alkoxy are optionally substituted with hydroxy, halo, oxo, NR⁸R⁹, ═NR¹⁰, COOC₁-C₆ alkyl, C₆-C₁₀ aryl, and CONR⁸R⁹;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO (5- to 10-membered heteroaryl), OCO (3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO (5- to 10-membered heteroaryl), OCO (3- to 7-membered heterocycloalkyl), NHCOC₁-C₆ alkyl, NHCOC₆-C₁₀ aryl, NHCO (5- to 10-membered heteroaryl), NHCO (3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five-to-seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO (5- to 10-membered heteroaryl) and NHCO (3- to 7-membered heterocycloalkyl) are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
each of R⁴ and R⁵ is independently selected from hydrogen and C₁-C₆ alkyl;
R₁₀ is C₁-C₆ alkyl;
each of R⁸ and R⁹ at each occurrence is independently selected from hydrogen, C₁-C₆ alkyl, NH—(C═NR¹³)NR¹¹R¹²; S(O₂)C₁-C₆ alkyl, S(O₂)NR¹¹R¹², COR¹³; CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to;
R¹³ is C₁-C₆ alkyl, C₆-C₁₀ aryl, or 5- to 10-membered heteroaryl;
each of R¹¹ and R¹² at each occurrence is independently selected from hydrogen and C₁-C₆ alkyl;
R³ is selected from hydrogen, cyano, hydroxy, C₁-C₆ alkoxy, C₁-C₆ alkyl, and 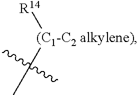
wherein the C₁-C₂ alkylene group is optionally substituted by oxo;
R¹⁴ is hydrogen, C₁-C₆ alkyl, 5- to 10-membered monocyclic or bicyclic heteroaryl or C₆-C₁₀ monocyclic or bicyclic aryl, wherein each C₁-C₆ alkyl, aryl or heteroaryl is optionally independently substituted with 1 or 2 R⁶,
or a pharmaceutically acceptable salt thereof.

40. The method according to claim 39, wherein the NLRP3 antagonist is at least one compound selected from the group consisting of compounds in Table 11, Table 12, Table 13, Table 14, and pharmaceutically acceptable salts thereof.


SEQUENCE LISTING (92 sequences)

SEQ ID NO: 1 - DNA, 675 bp, Homo sapiens
atggagaagctgttgtgtttcttggtcttgaccagcctctctcatgcttttggccagacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctga

SEQ ID NO: 2 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 3 - DNA, 675 bp, Homo sapiens; identical to SEQ ID NO: 1

SEQ ID NO: 4 - protein, 224 aa, Homo sapiens; identical to SEQ ID NO: 2

SEQ ID NO: 5 - DNA, 276 bp, Homo sapiens
atggagaagctgttgtgtttcttggtcttgaccagcctctctcatgcttttggccagacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtggtcctaatgtcctgaactggcgggcactgaagtatgaagtgcaaggcgaagtgttcaccaaaccccagctgtggccctga

SEQ ID NO: 6 - protein, 91 aa, Homo sapiens
MEKLLCFLVLTSLSHAFGQTDMSRKAFVFPKESDTSYVSLKAPLTKPLKAFTVCLHFYTELSSTRGPNVLNWRALKYEVQGEVFTKPQLWP

SEQ ID NO: 7 - DNA, 369 bp, Homo sapiens
atgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggctgcagaagtgatcagcgatgccagagagaatatccagagattctttggccatggtgcggaggactcgctggctgatcaggctgccaatgaatggggcaggagtggcaaagaccccaatcacttccgacctgctggcctgcctgagaaatactga

SEQ ID NO: 8 - protein, 122 aa, Homo sapiens
MKLLTGLVFCSLVLGVSSRSFFSFLGEAFDGARDMWRAYSDMREANYIGSDKYFHARGNYDAAKRGPGGAWAAEVISDARENIQRFFGHGAEDSLADQAANEWGRSGKDPNHFRPAGLPEKY

SEQ ID NO: 9 - DNA, 369 bp, Homo sapiens; identical to SEQ ID NO: 7

SEQ ID NO: 10 - protein, 122 aa, Homo sapiens; identical to SEQ ID NO: 8

SEQ ID NO: 11 - DNA, 369 bp, Homo sapiens; identical to SEQ ID NO: 7

SEQ ID NO: 12 - protein, 122 aa, Homo sapiens; identical to SEQ ID NO: 8

SEQ ID NO: 13 - DNA, 1221 bp, Homo sapiens
atgagtgccctgggagctgtcattgccctcctgctctggggacagctttttgcagtggactcaggcaatgatgtcacggatatcgcagatgacggctgcccgaagccccccgagattgcacatggctatgtggagcactcggttcgctaccagtgtaagaactactacaaactgcgcacagaaggagatggagtatacaccttaaatgataagaagcagtggataaataaggctgttggagataaacttcctgaatgtgaagcagatgacggctgcccgaagccccccgagattgcacatggctatgtggagcactcggttcgctaccagtgtaagaactactacaaactgcgcacagaaggagatggagtgtacaccttaaacaatgagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggtgccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggttgttctacaccctaactactcccaggtagatattgggctcatcaaactcaaacagaaggtgtctgttaatgagagagtgatgcccatctgcctaccttcaaaggattatgcagaagtagggcgtgtgggttatgtttctggctgggggcgaaatgccaattttaaatttactgaccatctgaagtatgtcatgctgcctgtggctgaccaagaccaatgcataaggcattatgaaggcagcacagtccccgaaaagaagacaccgaagagccctgtaggggtgcagcccatactgaatgaacacaccttctgtgctggcatgtctaagtaccaagaagacacctgctatggcgatgcgggcagtgcctttgccgttcacgacctggaggaggacacctggtatgcgactgggatcttaagctttgataagagctgtgctgtggctgagtatggtgtgtatgtgaaggtgacttccatccaggactgggttcagaagaccatagctgagaactaa

SEQ ID NO: 14 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 15 - DNA, 1044 bp, Homo sapiens
atgagtgccctgggagctgtcattgccctcctgctctggggacagctttttgcagtggactcaggcaatgatgtcacggatatcgcagatgacggctgcccgaagccccccgagattgcacatggctatgtggagcactcggttcgctaccagtgtaagaactactacaaactgcgcacagaaggagatggagtgtacaccttaaacaatgagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggtgccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggttgttctacaccctaactactcccaggtagatattgggctcatcaaactcaaacagaaggtgtctgttaatgagagagtgatgcccatctgcctaccttcaaaggattatgcagaagtagggcgtgtgggttatgtttctggctgggggcgaaatgccaattttaaatttactgaccatctgaagtatgtcatgctgcctgtggctgaccaagaccaatgcataaggcattatgaaggcagcacagtccccgaaaagaagacaccgaagagccctgtaggggtgcagcccatactgaatgaacacaccttctgtgctggcatgtctaagtaccaagaagacacctgctatggcgatgcgggcagtgcctttgccgttcacgacctggaggaggacacctggtatgcgactgggatcttaagctttgataagagctgtgctgtggctgagtatggtgtgtatgtgaaggtgacttccatccaggactgggttcagaagaccatagctgagaactaa

SEQ ID NO: 16 - protein, 347 aa, Homo sapiens
MSALGAVIALLLWGQLFAVDSGNDVTDIADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 17 - DNA, 1044 bp, Homo sapiens
atgagtgccctgggagctgtcattgccctcctgctctggggacagctttttgcagtggactcaggcaatgatgtcacggatatcgcagatgacggctgcccgaagccccccgagattgcacatggctatgtggagcactcggttcgctaccagtgtaagaactactacaaactgcgcacagaaggagatggagtatacaccttaaatgataagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggtgccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggttgttctacaccctaactactcccaggtagatattgggctcatcaaactcaaacagaaggtgtctgttaatgagagagtgatgcccatctgcctaccttcaaaggattatgcagaagtagggcgtgtgggttatgtttctggctgggggcgaaatgccaattttaaatttactgaccatctgaagtatgtcatgctgcctgtggctgaccaagaccaatgcataaggcattatgaaggcagcacagtccccgaaaagaagacaccgaagagccctgtaggggtgcagcccatactgaatgaacacaccttctgtgctggcatgtctaagtaccaagaagacacctgctatggcgatgcgggcagtgcctttgccgttcacgacctggaggaggacacctggtatgcgactgggatcttaagctttgataagagctgtgctgtggctgagtatggtgtgtatgtgaaggtgacttccatccaggactgggttcagaagaccatagctgagaactaa

SEQ ID NO: 18 - protein, 347 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 19 - DNA, 3198 bp, Homo sapiens
atgaagattttgatacttggtatttttctgtttttatgtagtaccccagcctgggcgaaagaaaagcattattacattggaattattgaaacgacttgggattatgcctctgaccatggggaaaagaaacttatttctgttgacacggaacattccaatatctatcttcaaaatggcccagatagaattgggagactatataagaaggccctttatcttcagtacacagatgaaacctttaggacaactatagaaaaaccggtctggcttgggtttttaggccctattatcaaagctgaaactggagataaagtttatgtacacttaaaaaaccttgcctctaggccctacacctttcattcacatggaataacttactataaggaacatgagggggccatctaccctgataacaccacagattttcaaagagcagatgacaaagtatatccaggagagcagtatacatacatgttgcttgccactgaagaacaaagtcctggggaaggagatggcaattgtgtgactaggatttaccattcccacattgatgctccaaaagatattgcctcaggactcatcggacctttaataatctgtaaaaaagattctctagataaagaaaaagaaaaacatattgaccgagaatttgtggtgatgttttctgtggtggatgaaaatttcagctggtacctagaagacaacattaaaacctactgctcagaaccagagaaagttgacaaagacaacgaagacttccaggagagtaacagaatgtattctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacagaaagatgtagacaaggaattctatttgtttcctacagtatttgatgagaatgagagtttactcctggaagataatattagaatgtttacaactgcacctgatcaggtggataaggaagatgaagactttcaggaatctaataaaatgcactccatgaatggattcatgtatgggaatcagccgggtctcactatgtgcaaaggagattcggtcgtgtggtacttattcagcgccggaaatgaggccgatgtacatggaatatacttttcaggaaacacatatctgtggagaggagaacggagagacacagcaaacctcttccctcaaacaagtcttacgctccacatgtggcctgacacagaggggacttttaatgttgaatgccttacaactgatcattacacaggcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaatgtttcaaatgcatttttagataagggagagttttacataggctcaaagtacaagaaagttgtgtatcggcagtatactgatagcacattccgtgttccagtggagagaaaagctgaagaagaacatctgggaattctaggtccacaacttcatgcagatgttggagacaaagtcaaaattatctttaaaaacatggccacaaggccctactcaatacatgcccatggggtacaaacagagagttctacagttactccaacattaccaggtgaaactctcacttacgtatggaaaatcccagaaagatctggagctggaacagaggattctgcttgtattccatgggcttattattcaactgtggatcaagttaaggacctctacagtggattaattggccccctgattgtttgtcgaagaccttacttgaaagtattcaatcccagaaggaaactggaatttgcccttctgtttctagtttttgatgagaatgaatcttggtacttagatgacaacatcaaaacatactctgatcaccccgagaaagtaaacaaagatgatgaggaattcatagaaagcaataaaatgcatgctattaatggaagaatgtttggaaacctacaaggcctcacaatgcacgtgggagatgaagtcaactggtatctgatgggaatgggcaatgaaatagacttacacactgtacattttcacggccatagcttccaatacaagcacaggggagtttatagttctgatgtctttgacattttccctggaacataccaaaccctagaaatgtttccaagaacacctggaatttggttactccactgccatgtgaccgaccacattcatgctggaatggaaaccacttacaccgttctacaaaatgaagacaccaaatctggctga

SEQ ID NO: 20 - protein, 1065 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 21 - DNA, 639 bp, Homo sapiens
atgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtag

SEQ ID NO: 22 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 23 - DNA, 411 bp, Homo sapiens
atgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtag

SEQ ID NO: 24 - protein, 136 aa, Homo sapiens
MCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 25 - DNA, 300 bp, Homo sapiens
atgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataa

SEQ ID NO: 26 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 27 - DNA, 288 bp, Homo sapiens
atgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagaggtaa

SEQ ID NO: 28 - protein, 95 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKR

SEQ ID NO: 29 - DNA, 1059 bp, Homo sapiens
atgaacactacatcttctgcagcacccccctcactaggtgtagagttcatctctctgctggctatcatcctgctgtcagtggcgctggctgtggggcttcccggcaacagctttgtggtgtggagtatcctgaaaaggatgcagaagcgctctgtcactgccctgatggtgctgaacctggccctggccgacctggccgtattgctcactgctccctttttccttcacttcctggcccaaggcacctggagttttggactggctggttgccgcctgtgtcactatgtctgcggagtcagcatgtacgccagcgtcctgcttatcacggccatgagtctagaccgctcactggcggtggcccgcccctttgtgtcccagaagctacgcaccaaggcgatggcccggcgggtgctggcaggcatctgggtgttgtcctttctgctggccacacccgtcctcgcgtaccgcacagtagtgccctggaaaacgaacatgagcctgtgcttcccgcggtaccccagcgaagggcaccgggccttccatctaatcttcgaggctgtcacgggcttcctgctgcccttcctggctgtggtggccagctactcggacatagggcgtcggctacaggcccggcgcttccgccgcagccgccgcaccggccgcctggtggtgctcatcatcctgaccttcgccgccttctggctgccctaccacgtggtgaacctggctgaggcgggccgcgcgctggccggccaggccgccgggttagggctcgtggggaagcggctgagcctggcccgcaacgtgctcatcgcactcgccttcctgagcagcagcgtgaaccccgtgctgtacgcgtgcgccggcggcggcctgctgcgctcggcgggcgtgggcttcgtcgccaagctgctggagggcacgggctccgaggcgtccagcacgcgccgcgggggcagcctgggccagaccgctaggagcggccccgccgctctggagcccggcccttccgagagcctcactgcctccagccctctcaagttaaacgaactgaactag

SEQ ID NO: 30 - protein, 352 aa, Homo sapiens
MNTTSSAAPPSLGVEFISLLAIILLSVALAVGLPGNSFVVWSILKRMQKRSVTALMVLNLALADLAVLLTAPFFLHFLAQGTWSFGLAGCRLCHYVCGVSMYASVLLITAMSLDRSLAVARPFVSQKLRTKAMARRVLAGIWVLSFLLATPVLAYRTVVPWKTNMSLCFPRYPSEGHRAFHLIFEAVTGFLLPFLAVVASYSDIGRRLQARRFRRSRRTGRLVVLIILTFAAFWLPYHVVNLAEAGRALAGQAAGLGLVGKRLSLARNVLIALAFLSSSVNPVLYACAGGGLLRSAGVGFVAKLLEGTGSEASSTRRGGSLGQTARSGPAALEPGPSESLTASSPLKLNELN

SEQ ID NO: 31 - DNA, 1119 bp, Homo sapiens
atgaacactacatcttctgcagcacccccctcactaggtgtagagttcatctctctgctggctatcatcctgctgtcagtggcgctggctgtggggcttcccggcaacagctttgtggtgtggagtatcctgaaaaggatgcagaagcgctctgtcactgccctgatggtgctgaacctggccctggccgacctggccgtattgctcactgctccctttttccttcacttcctggcccaaggcacctggagttttggactggctggttgccgcctgtgtcactatgtctgcggagtcagcatgtacgccagcgtcctgcttatcacggccatgagtctagaccgctcactggcggtggcccgcccctttgtgtcccagaagctacgcaccaaggcgatggcccggcgggtgctggcaggcatctgggtgttgtcctttctgctggccacacccgtcctcgcgtaccgcacagtagtgccctggaaaacgaacatgagcctgtgcttcccgcggtaccccagcgaagggcaccgggccttccatctaatcttcgaggctgtcacgggcttcctgctgcccttcctggctgtggtggccagctactcggacatagggcgtcggctacaggcccggcgcttccgccgcagccgccgcaccggccgcctggtggtgctcatcatcctgaccttcgccgccttctggctgccctaccacgtggtgaacctggctgaggcgggccgcgcgctggccggccaggccgccgggttagggctcgtggggaagcggctgagcctggcccgcaacgtgctcatcgcactcgccttcctgagcagcagcgtgaaccccgtgctgtacgcgtgcgccggcggcggcctgctgcgctcggcgggcgtgggcttcgtcgccaagctgctggagggcacgggctccgaggcgtccagcacgcgccgcgggggcagcctgggccagaccgctaggagcggccccgccgctctggagcccggcccttccgagagcctcactgcctccagccctctcaagttaaacgaactgaactaggcctggtggaaggaggcgcactttcctcctggcagaatgctagctctgagccagttcagt

SEQ ID NO: 32 - protein, 352 aa, Homo sapiens; identical to SEQ ID NO: 30

SEQ ID NO: 33 - DNA, 2238 bp, Homo sapiens
atgggggttcccttcttctcttctctcagatgcatggtggacttaggaccttgctgggctgggggtctcactgcagagatgaagctgcttctggccctagcagggctcctggccattctggccacgccccagccctctgaaggtgctgctccagctgtcctgggggaggtggacacctcgttggtgctgagctccatggaggaggccaagcagctggtggacaaggcctacaaggagcggcgggaaagcatcaagcagcggcttcgcagcggctcagccagccccatggaactcctatcctacttcaagcagccggtggcagccaccaggacggcggtgagggccgctgactacctgcacgtggctctagacctgctggagaggaagctgcggtccctgtggcgaaggccattcaatgtcactgatgtgctgacgcccgcccagctgaatgtgttgtccaagtcaagcggctgcgcctaccaggacgtgggggtgacttgcccggagcaggacaaataccgcaccatcaccgggatgtgcaacaacagacgcagccccacgctgggggcctccaaccgtgcctttgtgcgctggctgccggcggagtatgaggacggcttctctcttccctacggctggacgcccggggtcaagcgcaacggcttcccggtggctctggctcgcgcggtctccaacgagatcgtgcgcttccccactgatcagctgactccggaccaggagcgctcactcatgttcatgcaatggggccagctgttggaccacgacctcgacttcacccctgagccggccgcccgggcctccttcgtcactggcgtcaactgcgagaccagctgcgttcagcagccgccctgcttcccgctcaagatcccgcccaatgacccccgcatcaagaaccaagccgactgcatcccgttcttccgctcctgcccggcttgccccgggagcaacatcaccatccgcaaccagatcaacgcgctcacttccttcgtggacgccagcatggtgtacggcagcgaggagcccctggccaggaacctgcgcaacatgtccaaccagctggggctgctggccgtcaaccagcgcttccaagacaacggccgggccctgctgccctttgacaacctgcacgatgacccctgtctcctcaccaaccgctcagcgcgcatcccctgcttcctggcaggggacacccgttccagtgagatgcccgagctcacctccatgcacaccctcttacttcgggagcacaaccggctggccacagagctcaagagcctgaaccctaggtgggatggggagaggctctaccaggaagcccggaagatcgtgggggccatggtccagatcatcacttaccgggactacctgcccctggtgctggggccaacggccatgaggaagtacctgcccacgtaccgttcctacaatgactcagtggacccacgcatcgccaacgtcttcaccaatgccttccgctacggccacaccctcatccaacccttcatgttccgcctggacaatcggtaccagcccatggaacccaacccccgtgtccccctcagcagggtcttttttgcctcctggagggtcgtgctggaaggtggcattgaccccatcctccggggcctcatggccacccctgccaagctgaatcgtcagaaccaaattgcagtggatgagatccgggagcgattgtttgagcaggtcatgaggattgggctggacctgcctgctctgaacatgcagcgcagcagggaccacggcctcccaggatacaatgcctggaggcgcttctgtgggctcccgcagcctgaaactgtgggccagctgggcacggtgctgaggaacctgaaattggcgaggaaactgatggagcagtatggcacgcccaacaacatcgacatctggatgggcggcgtgtccgagcctctgaagcgcaaaggccgcgtgggcccactcctcgcctgcatcatcggtacccagttcaggaagctccgggatggtgatcggttttggtgggagaacgagggtgtgttcagcatgcagcagcgacaggccctggcccagatctcattgccccggatcatctgcgacaacacaggcatcaccaccgtgtctaagaacaacatcttcatgtccaactcatatccccgggactttgtcaactgcagtacacttcctgcattgaacctggcttcctggagggaagcctcctag

SEQ ID NO: 34 - protein, 745 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 35 - protein, 976 aa, Homo sapiens
MKMASTRCKLARYLEDLEDVDLKKFKMHLEDYPPQKGCIPLPRGQTEKADHVDLATLMIDFNGEEKAWAMAVWIFAAINRRDLYEKAKRDEPKWGSDNARVSNPTVICQEDSIEEEWMGLLEYLSRISICKMKKDYRKKYRKYVRSRFQCIEDRNARLGESVSLNKRYTRLRLIKEHRSQQEREQELLAIGKTKTCESPVSPIKMELLFDPDDEHSEPVHTVVFQGAAGIGKTILARKMMLDWASGTLYQDRFDYLFYIHCREVSLVTQRSLGDLIMSCCPDPNPPIHKIVRKPSRILFLMDGFDELQGAFDEHIGPLCTDWQKAERGDILLSSLIRKKLLPEASLLITTRPVALEKLQHLLDHPRHVEILGFSEAKRKEYFFKYFSDEAQARAAFSLIQENEVLFTMCFIPLVCWIVCTKILFEESDLRNHGLQKADVSAFLRMNLFQKEVDCEKFYSFIHMTFQEFFAAMYYLLEEEKEGRTNVPGSRLKLPSRDVTVLLENYGKFEKGYLIFVVRFLFGLVNQERTSYLEKKLSCKISQQIRLELLKWIEVKAKAKKLQIQPSQLELFYCLYEMQEEDFVQRAMDYFPKIEINLSTRMDHMVSSFCIENCHRVESLSLGFLHNMPKEEEEEEKEGRHLDMVQCVLPSSSHAACSHGLVNSHLTSSFCRGLFSVLSTSQSLTELDLSDNSLGDPGMRVLCETLQHPGCNIRRLWLGRCGLSHECCFDISLVLSSNQKLVELDLSDNALGDFGIRLLCVGLKHLLCNLKKLWLVSCCLTSACCQDLASVLSTSHSLTRLYVGENALGDSGVAILCEKAKNPQCNLQKLGLVNSGLTSVCCSALSSVLSTNQNLTHLYLRGNTLGDKGIKLLCEGLLHPDCKLQVLELDNCNLTSHCCWDLSTLLTSSQSLRKLSLGNNDLGDLGVMMFCEVLKQQSCLLQNLGLSEMYFNYETKSALETLQEEKPELTVVFEPSW

SEQ ID NO: 36 - DNA, 702 bp, Homo sapiens
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtga

SEQ ID NO: 37 - DNA, 1368 bp, Homo sapiens
atgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatga

SEQ ID NO: 38 - DNA, 281 bp, Homo sapiens
attcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaa

SEQ ID NO: 39 - DNA, 939 bp, Homo sapiens
atggcagctgggcaaaatgggcacgaagagtgggtgggcagcgcatacctgtttgtggagtcctcgctggacaaggtggtcctgtcggatgcctacgcgcacccccagcagaaggtggcagtgtacagggctctgcaggctgccttggcagagagcggcgggagcccggacgtgctgcagatgctgaagatccaccgcagcgacccgcagctgatcgtgcagctgcgattctgcgggcggcagccctgtggccgcttcctccgcgcctaccgcgagggggcgctgcgcgccgcgctgcagaggagcctggcggccgcgctcgcccagcactcggtgccgctgcaactggagctgcgcgccggcgccgagcggctggacgctttgctggcggacgaggagcgctgtttgagttgcatcctagcccagcagcccgaccggctccgggatgaagaactggctgagctggaggatgcgctgcgaaatctgaagtgcggctcgggggcccggggtggcgacggggaggtcgcttcggcccccttgcagcccccggtgccctctctgtcggaggtgaagccgccgccgccgccgccacctgcccagacttttctgttccagggtcagcctgtagtgaatcggccgctgagcctgaaggaccaacagacgttcgcgcgctctgtgggtctcaaatggcgcaaggtggggcgctcactgcagcgaggctgccgggcgctgcgggacccggcgctggactcgctggcctacgagtacgagcgcgagggactgtacgagcaggccttccagctgctgcggcgcttcgtgcaggccgagggccgccgcgccacgctgcagcgcctggtggaggcactcgaggagaacgagctcaccagcctggcagaggacttgctgggcctgaccgatcccaatggcggcctggcctag

SEQ ID NO: 40 - DNA, 1506 bp, Homo sapiens
atggctgcagctagcgtgaccccccctggctccctggagttgctacagcccggcttctccaagaccctcctggggaccaagctggaagccaagtacctgtgctccgcctgcagaaacgtcctccgcaggcccttccaggcgcagtgtggccaccggtactgctccttctgcctggccagcatcctcagctctgggcctcagaactgtgctgcctgtgttcacgagggcatatatgaagaaggcatttctattttagaaagcagttcggccttcccagataatgctgcccgcagggaggtggagagcctgccggccgtctgtcccagtgatggatgcacctggaaggggaccctgaaagaatacgagagctgccacgaaggccgctgcccgctcatgctgaccgaatgtcccgcgtgcaaaggcctggtccgccttggtgaaaaggagcgccacctggagcacgagtgcccggagagaagcctgagctgccggcattgccgggcaccctgctgcggagcagacgtgaaggcgcaccacgaggtctgccccaagttccccttaacttgtgacggctgcggcaagaagaagatcccccgggagaagtttcaggaccacgtcaagacttgtggcaagtgtcgagtcccttgcagattccacgccatcggctgcctcgagacggtagagggtgagaaacagcaggagcacgaggtgcagtggctgcgggagcacctggccatgctactgagctcggtgctggaggcaaagcccctcttgggagaccagagccacgcggggtcagagctcctgcagaggtgcgagagcctggagaagaagacggccacttttgagaacattgtctgcgtcctgaaccgggaggtggagagggtggccatgactgccgaggcctgcagccggcagcaccggctggaccaagacaagattgaagccctgagtagcaaggtgcagcagctggagaggagcattggcctcaaggacctggcgatggctgacttggagcagaaggtcttggagatggaggcatccacctacgatggggtcttcatctggaagatctcagacttcgccaggaagcgccaggaagctgtggctggccgcatacccgccatcttctccccagccttctacaccagcaggtacggctacaagatgtgtctgcgtatctacctgaacggcgacggcaccgggcgaggaacacacctgtccctcttctttgtggtgatgaagggcccgaatgacgccctgctgcggtggcccttcaaccagaaggtgaccttaatgctgctcgaccagaataaccgggagcacgtgattgacgccttcaggcccgacgtgacttcatcctcttttcagaggccagtcaacgacatgaacatcgcaagcggctgccccctcttctgccccgtctccaagatggaggcaaagaattcctacgtgcgggacgatgccatcttcatcaaggccattgtggacctgacagggctctaa

SEQ ID NO: 41 - DNA, 1044 bp, Homo sapiens
atggaaacacccttctacggcgatgaggcgctgagcggcctgggcggcggcgccagtggcagcggcggcagcttcgcgtccccgggccgcttgttccccggggcgcccccgacggccgcggccggcagcatgatgaagaaggacgcgctgacgctgagcctgagtgagcaggtggcggcagcgctcaagcctgcggccgcgccgcctcctacccccctgcgcgccgacggcgcccccagcgcggcaccccccgacggcctgctcgcctctcccgacctggggctgctgaagctggcctcccccgagctcgagcgcctcatcatccagtccaacgggctggtcaccaccacgccgacgagctcacagttcctctaccccaaggtggcggccagcgaggagcaggagttcgccgagggcttcgtcaaggccctggaggatttacacaagcagaaccagctcggcgcgggcgcggccgctgccgccgccgccgccgccgccggggggccctcgggcacggccacgggctccgcgccccccggcgagctggccccggcggcggccgcgcccgaagcgcctgtctacgcgaacctgagcagctacgcgggcggcgccgggggcgcggggggcgccgcgacggtcgccttcgctgccgaacctgtgcccttcccgccgccgccacccccaggcgcgttggggccgccgcgcctggctgcgctcaaggacgagccacagacggtgcccgacgtgccgagcttcggcgagagcccgccgttgtcgcccatcgacatggacacgcaggagcgcatcaaggcggagcgcaagcggctgcgcaaccgcatcgccgcctccaagtgccgcaagcgcaagctggagcgcatctcgcgcctggaagagaaagtgaagaccctcaagagtcagaacacggagctggcgtccacggcgagcctgctgcgcgagcaggtggcgcagctcaagcagaaagtcctcagccacgtcaacagcggctgccagctgctgccccagcaccaggtgcccgcgtactga

SEQ ID NO: 42 - DNA, 4124 bp, Homo sapiens
atgagcacggaggcggacgagggcatcactttctctgtgccacccttcgccccctcgggcttctgcaccatccccgagggcggcatctgcaggaggggaggagcggcggcggtgggcgagggcgaggagcaccagctgccaccgccgccgccgggcagtttctggaacgtggagagcgccgctgcccctggcatcggttgtccggcggccacctcctcgagcagtgccacccgaggccggggcagctctgttggcgggggcagccgacggaccacggtggcatatgtgatcaacgaagcgagccaagggcaactggtggtggccgagagcgaggccctgcagagcttgcgggaggcgtgcgagacagtgggcgccaccctggaaccctgcattttgggaaactcgactttggagaaaccaccgtgctggaccgcttttacaatgcagatattgcggtggtggagatgagcgatgccttccggcagccgtccttgttttaccaccttggggtgagagaaagtttcagcatggccaacaacatcatcctctactgcgatactaactcggactctctgcagtcactgaaggaaatcatttgccagaagaatactatgtgcactgggaactacacctttgttccttacatgataactccacataacaaagtctactgctgtgacagcagcttcatgaaggggttgacagagctcatgcaaccgaacttcgagctgcttcttggacccatctgcttacctcttgtggatcgttttattcaacttttgaaggtggcacaagcaagttctagccagtacttccgggaatctatactcaatgacatcaggaaagctcgtaatttatacactggtaaagaattggcagctgagttggcaagaattcggcagcgagtagataatatcgaagtcttgacagcagatattgtcataaatctgttactttcctacagagatatccaggactatgattctattgtgaagctggtagagactttagaaaaactgccaacctttgatttggcctcccatcaccatgtgaagtttcattatgcatttgcactgaataggagaaatctccctggtgacagagcaaaagctcttgatattatgattcccatggtgcaaagcgaaggacaagttgcttcagatatgtattgcctagttggtcgaatctacaaagatatgtttttggactctaatttcacggacactgaaagcagagaccatggagcttcttggttcaaaaaggcatttgaatctgagccaacactacagtcaggaattaattatgcggtcctcctcctggcagctggacaccagtttgaatcttcctttgagctccggaaagttggggtgaagctaagtagtcttcttggtaaaaagggaaacttggaaaaactccagagctactgggaagttggattttttctgggggccagcgtcctagccaatgaccacatgagagtcattcaagcatctgaaaagctttttaaactgaagacaccagcatggtacctcaagtctattgtagagacaattttgatatataagcattttgtgaaactgaccacagaacagcctgtggccaagcaagaacttgtggacttttggatggatttcctggtcgaggccacaaagacagatgttactgtggttaggtttccagtattaatattagaaccaaccaaaatctatcaaccttcttatttgtctatcaacaatgaagttgaggaaaagacaatctctatttggcacgtgcttcctgatgacaagaaaggtatacatgagtggaattttagtgcctcttctgtcaggggagtgagtatttctaaatttgaagaaagatgctgctttctttatgtgcttcacaattctgatgatttccaaatctatttctgtacagaacttcattgtaaaaagttttttgagatggtgaacaccattaccgaagagaaggggagaagcacagaggaaggagactgtgaaagtgacttgctggagtatgactatgaatatgatgaaaatggtgacagagtcgttttaggaaaaggcacttatgggatagtctacgcaggtcgggacttgagcaaccaagtcagaattgctattaaggaaatcccagagagagacagcagatactctcagcccctgcatgaagaaatagcattgcataaacacctgaagcacaaaaatattgtccagtatctgggctctttcagtgagaatggtttcattaaaatcttcatggagcaggtccctggaggaagtctttctgctctccttcgttccaaatggggtccattaaaagacaatgagcaaacaattggcttttatacaaagcaaatactggaaggattaaaatatctccatgacaatcagatagttcaccgggacataaagggtgacaatgtgttgattaatacctacagtggtgttctcaagatctctgacttcggaacatcaaagaggcttgctggcataaacccctgtactgaaacttttactggtaccctccagtatatggcaccagaaataatagataaaggaccaagaggctacggaaaagcagcagacatctggtctctgggctgtacaatcattgaaatggccacaggaaaacccccattttatgaactgggagaaccacaagcagctatgttcaaggtgggaatgtttaaagtccaccctgagatcccagagtccatgtctgcagaggccaaggcattcatactgaaatgttttgaaccagatcctgacaagagagcctgtgctaacgacttgcttgttgatgagtttttaaaagtttcaagcaaaaagaaaaagacacaacctaagctttcagctctttcagctggatcaaatgaatatctcaggagtatatccttgccggtacctgtgctggtggaggacaccagcagcagcagtgagtacggctcagtttcacccgacacggagttgaaagtggaccccttctctttcaaaacaagagccaagtcctgcggagaaagagatgtcaagggaattcggacactctttttgggcattccagatgagaattttgaagatcacagtgctcctccttcccctgaagaaaaagattctggattcttcatgctgaggaaggacagtgagaggcgagctacccttcacaggatcctgacggaagaccaagacaaaattgtgagaaacctaatggaatctttagctcagggggctgaagaaccgaaactaaaatgggaacacatcacaaccctcattgcaagcctcagagaatttgtgagatccactgaccgaaaaatcatagccaccacactgtcaaagctgaaactggagctggacttcgacagccatggcattagccaagtccaggtggtactctttggttttcaagatgctgtcaataaagttcttcggaatcataacatcaagccgcactggatgtttgccttagacagtatcattcggaaggcggtacagacagccattaccatcctggttccagaactaaggccacatttcagccttgcatctgagagtgatactgctgatcaagaagacttggatgtagaagatgaccatgaggaacagccttcaaatcaaactgtccgaagacctcaggctgtcattgaagatgctgtggctacctcaggcgtgagcacgctcagttctactgtgtctcatgattcccagagtgctcaccggtcactgaatgtacagcttggaaggatgaaaatagaaaccaatagattactggaagaattggttcggaaagagaaagaattacaagcactccttcatcgagctattgaagaaaaagaccaagaaattaaacacctgaagcttaagtcccaacccatagaaattcctgaattgcctgtatttcatctaaattcttctggcacaaatactgaagattctgaacttaccgactggctgagagtgaatggagctgatgaagacactataagccggtttttggctgaagattatacactattggatgttctctactatgttacacgtgatgacttaaaatgcttgagactaaggggagggatgctgtgcacactgtggaaggctatcattgactttcgaaacaaacagacttga

SEQ ID NO: 43 - DNA, 1128 bp, Homo sapiens
atggagcgcgcgtcctgcttgttgctgctgctgctgccgctggtgcacgtctctgcgaccacgccagaaccttgtgagctggacgatgaagatttccgctgcgtctgcaacttctccgaacctcagcccgactggtccgaagccttccagtgtgtgtctgcagtagaggtggagatccatgccggcggtctcaacctagagccgtttctaaagcgcgtcgatgcggacgccgacccgcggcagtatgctgacacggtcaaggctctccgcgtgcggcggctcacagtgggagccgcacaggttcctgctcagctactggtaggcgccctgcgtgtgctagcgtactcccgcctcaaggaactgacgctcgaggacctaaagataaccggcaccatgcctccgctgcctctggaagccacaggacttgcactttccagcttgcgcctacgcaacgtgtcgtgggcgacagggcgttcttggctcgccgagctgcagcagtggctcaagccaggcctcaaggtactgagcattgcccaagcacactcgcctgccttttcctgcgaacaggttcgcgccttcccggcccttaccagcctagacctgtctgacaatcctggactgggcgaacgcggactgatggcggctctctgtccccacaagttcccggccatccagaatctagcgctgcgcaacacaggaatggagacgcccacaggcgtgtgcgccgcactggcggcggcaggtgtgcagccccacagcctagacctcagccacaactcgctgcgcgccaccgtaaaccctagcgctccgagatgcatgtggtccagcgccctgaactccctcaatctgtcgttcgctgggctggaacaggtgcctaaaggactgccagccaagctcagagtgctcgatctcagctgcaacagactgaacagggcgccgcagcctgacgagctgcccgaggtggataacctgacactggacgggaatcccttcctggtccctggaactgccctcccccacgagggctcaatgaactccggcgtggtcccagcctgtgcacgttcgaccctgtcggtgggggtgtcgggaaccctggtgctgctccaaggggcccggggctttgcctaa

SEQ ID NO: 44 - DNA, 1140 bp, Homo sapiens
atggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgagccagtggccgaggagcccttcaccttcgccatggagctggatgacctacctaaggagcggctgaaggagctcatcttccaggagacagcacgcttccagcccggagtgctggaggccccctag

SEQ ID NO: 45 - DNA, 1083 bp, Homo sapiens
atggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaa

SEQ ID NO: 46 - DNA, 2265 bp, Homo sapiens
atgttttcaggggggtgtcatagccccgggtttggccgccccagccccgccttccccgccccggggagcccgccccctgccccgcgtccctgccgacaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatga

SEQ ID NO: 47 - DNA, 954 bp, Homo sapiens
atgttccaggcggccgagcgcccccaggagtgggccatggagggcccccgcgacgggctgaagaaggagcggctactggacgaccgccacgacagcggcctggactccatgaaagacgaggagtacgagcagatggtcaaggagctgcaggagatccgcctcgagccgcaggaggtgccgcgcggctcggagccctggaagcagcagctcaccgaggacggggactcgttcctgcacttggccatcatccatgaagaaaaggcactgaccatggaagtgatccgccaggtgaagggagacctggccttcctcaacttccagaacaacctgcagcagactccactccacttggctgtgatcaccaaccagccagaaattgctgaggcacttctgggagctggctgtgatcctgagctccgagactttcgaggaaatacccccctacaccttgcctgtgagcagggctgcctggccagcgtgggagtcctgactcagtcctgcaccaccccgcacctccactccatcctgaaggctaccaactacaatggccacacgtgtctacacttagcctctatccatggctacctgggcatcgtggagcttttggtgtccttgggtgctgatgtcaatgctcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgttatga

SEQ ID NO: 48 - DNA, 2139 bp, Homo sapiens
atggccggggggccgggcccgggggagcccgcagcccccggcgcccagcacttcttgtacgaggtgccgccctgggtcatgtgccgcttctacaaagtgatggacgccctggagcccgccgactggtgccagttcgccgccctgatcgtgcgcgaccagaccgagctgcggctgtgcgagcgctccgggcagcgcacggccagcgtcctgtggccctggatcaaccgcaacgcccgtgtggccgacctcgtgcacatcctcacgcacctgcagctgctccgtgcgcgggacatcatcacagcctggcaccctcccgccccgcttccgtccccaggcaccactgccccgaggcccagcagcatccctgcacccgccgaggccgaggcctggagcccccggaagttgccatcctcagcctccaccttcctctccccagcttttccaggctcccagacccattcagggcctgagctcggcctggtcccaagccctgcttccctgtggcctccaccgccatctccagccccttcttctaccaagccaggcccagagagctcagtgtccctcctgcagggagcccgcccctttccgttttgctggcccctctgtgagatttcccggggcacccacaacttctcggaggagctcaagatcggggagggtggctttgggtgcgtgtaccgggcggtgatgaggaacacggtgtatgctgtgaagaggctgaaggagaacgctgacctggagtggactgcagtgaagcagagcttcctgaccgaggtggagcagctgtccaggtttcgtcacccaaacattgtggactttgctggctactgtgctcagaacggcttctactgcctggtgtacggcttcctgcccaacggctccctggaggaccgtctccactgccagacccaggcctgcccacctctctcctggcctcagcgactggacatccttctgggtacagcccgggcaattcagtttctacatcaggacagccccagcctcatccatggagacatcaagagttccaacgtccttctggatgagaggctgacacccaagctgggagactttggcctggcccggttcagccgctttgccgggtccagccccagccagagcagcatggtggcccggacacagacagtgcggggcaccctggcctacctgcccgaggagtacatcaagacgggaaggctggctgtggacacggacaccttcagctttggggtggtagtgctagagaccttggctggtcagagggctgtgaagacgcacggtgccaggaccaagtatctgaaagacctggtggaagaggaggctgaggaggctggagtggctttgagaagcacccagagcacactgcaagcaggtctggctgcagatgcctgggctgctcccatcgccatgcagatctacaagaagcacctggaccccaggcccgggccctgcccacctgagctgggcctgggcctgggccagctggcctgctgctgcctgcaccgccgggccaaaaggaggcctcctatgacccaggtgtacgagaggctagagaagctgcaggcagtggtggcgggggtgcccgggcattcggaggccgccagctgcatccccccttccccgcaggagaactcctacgtgtccagcactggcagagcccacagtggggctgctccatggcagcccctggcagcgccatcaggagccagtgcccaggcagcagagcagctgcagagaggccccaaccagcccgtggagagtgacgagagcctaggcggcctctctgctgccctgcgctcctggcacttgactccaagctgccctctggacccagcacccctcagggaggccggctgtcctcagggggacacggcaggagaatcgagctgggggagtggcccaggatcccggcccacagccgtggaaggactggcccttggcagctctgcatcatcgtcgtcagagccaccgcagattatcatcaaccctgcccgacagaagatggtccagaagctggccctgtacgaggatggggccctggacagcctgcagctgctgtcgtccagctccctcccaggcttgggcctggaacaggacaggcaggggcccgaagaaagtgatgaatttcagagctga

SEQ ID NO: 49 - DNA, 1284 bp, Homo sapiens
atgagcagaagcaagcgtgacaacaatttttatagtgtagagattggagattctacattcacagtcctgaaacgatatcagaatttaaaacctataggctcaggagctcaaggaatagtatgcgcagcttatgatgccattcttgaaagaaatgttgcaatcaagaagctaagccgaccatttcagaatcagactcatgccaagcgggcctacagagagctagttcttatgaaatgtgttaatcacaaaaatataattggccttttgaatgttttcacaccacagaaatccctagaagaatttcaagatgtttacatagtcatggagctcatggatgcaaatctttgccaagtgattcagatggagctagatcatgaaagaatgtcctaccttctctatcagatgctgtgtggaatcaagcaccttcattctgctggaattattcatcgggacttaaagcccagtaatatagtagtaaaatctgattgcactttgaagattcttgacttcggtctggccaggactgcaggaacgagttttatgatgacgccttatgtagtgactcgctactacagagcacccgaggtcatccttggcatgggctacaaggaaaacgttgacatttggtcagttgggtgcatcatgggagaaatgatcaaaggtggtgttttgttcccaggtacagatcatattgatcagtggaataaagttattgaacagcttggaacaccatgtcctgaattcatgaagaaactgcaaccaacagtaaggacttacgttgaaaacagacctaaatatgctggatatagctttgagaaactcttccctgatgtccttttcccagctgactcagaacacaacaaacttaaagccagtcaggcaagggatttgttatccaaaatgctggtaatagatgcatctaaaaggatctctgtagatgaagctctccaacacccgtacatcaatgtctggtatgatccttctgaagcagaagctccaccaccaaagatccctgacaagcagttagatgaaagggaacacacaatagaagagtggaaagaattgatatataaggaagttatggacttggaggagagaaccaagaatggagttatacgggggcagccctctcctttaggtgcagcagtgatcaatggctctcagcatccatcatcatcgtcgtctgtcaatgatgtgtcttcaatgtcaacagatccgactttggcctctgatacagacagcagtctagaagcagcagctgggcctctgggctgctgtagatga

SEQ ID NO: 50 - DNA, 1446 bp, Homo sapiens
atgggggccttggccagagccctgccgtccatactgctggcattgctgcttacgtccaccccagaggctctgggtgccaaccccggcttggtcgccaggatcaccgacaagggactgcagtatgcggcccaggaggggctattagctctgcagagtgagctgctcaggatcacgctgcctgacttcaccggggacttgaggatcccccacgtcggccgtgggcgctatgagttccacagcctgaacatccacagctgtgagctgcttcactctgcgctgaggcctgtccctggccagggcctgagtctcagcatctccgactcctccatccgggtccagggcaggtggaaggtgcgcaagtcattcttcaaactacagggctcctttgatgtcagtgtcaagggcatcagcatttcggtcaacctcctgttgggcagcgagtcctccgggaggcccacagttactgcctccagctgcagcagtgacatcgctgacgtggaggtggacatgtcgggagacttggggtggctgttgaacctcttccacaaccagattgagtccaagttccagaaagtactggagagcaggatttgcgaaatgatccagaaatcggtgtcctccgatctacagccttatctccaaactctgccagttacaacagagattgacagtttcgccgacattgattatagcttagtggaagcccctcgggcaacagcccagatgctggaggtgatgtttaagggtgaaatctttcatcgtaaccaccgttctccagttaccctccttgctgcagtcatgagccttcctgaggaacacaacaaaatggtctactttgccatctcggattatgtcttcaacacggccagcctggtttatcatgaggaaggatatctgaacttctccatcacagatgacatgataccgcctgactctaatatccgactgaccaccaagtccttccgacccttcgtcccacggttagccaggctctaccccaacatgaacctggaactccagggatcagtgccctctgctccgctcctgaacttcagccctgggaatctgtctgtggacccctatatggagatagatgcctttgtgctcctgcccagctccagcaaggagcctgtcttccggctcagtgtggccactaatgtgtccgccaccttgaccttcaataccagcaagatcactgggttcctgaagccaggaaaggtaaaagtggaactgaaagaatccaaagttggactattcaatgcagagctgttggaagcgctcctcaactattacatccttaacaccctctaccccaagttcaatgataagttggccgaaggcttcccccttcctctgctgaagcgtgttcagctctacgaccttgggctgcagatccataaggacttcctgttcttgggtgccaatgtccaatacatgagagtttga

SEQ ID NO: 51 - DNA, 1182 bp, Homo sapiens
atgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaa

SEQ ID NO: 52 - DNA, 1203 bp, Homo sapiens
atgctggcccggaggaagccggtgctgccggcgctcaccatcaaccctaccatcgccgagggcccatcccctaccagcgagggcgcctccgaggcaaacctggtggacctgcagaagaagctggaggagctggaacttgacgagcagcagaagaagcggctggaagcctttctcacccagaaagccaaggtcggcgaactcaaagacgatgacttcgaaaggatctcagagctgggcgcgggcaacggcggggtggtcaccaaagtccagcacagaccctcgggcctcatcatggccaggaagctgatccaccttgagatcaagccggccatccggaaccagatcatccgcgagctgcaggtcctgcacgaatgcaactcgccgtacatcgtgggcttctacggggccttctacagtgacggggagatcagcatttgcatggaacacatggacggcggctccctggaccaggtgctgaaagaggccaagaggattcccgaggagatcctggggaaagtcagcatcgcggttctccggggcttggcgtacctccgagagaagcaccagatcatgcaccgagatgtgaagccctccaacatcctcgtgaactctagaggggagatcaagctgtgtgacttcggggtgagcggccagctcatcgactccatggccaactccttcgtgggcacgcgctcctacatggctccggagcggttgcagggcacacattactcggtgcagtcggacatctggagcatgggcctgtccctggtggagctggccgtcggaaggtaccccatccccccgcccgacgccaaagagctggaggccatctttggccggcccgtggtcgacggggaagaaggagagcctcacagcatctcgcctcggccgaggccccccgggcgccccgtcagcggtcacgggatggatagccggcctgccatggccatctttgaactcctggactatattgtgaacgagccacctcctaagctgcccaacggtgtgttcacccccgacttccaggagtttgtcaataaatgcctcatcaagaacccagcggagcgggcggacctgaagatgctcacaaaccacaccttcatcaagcggtccgaggtggaagaagtggattttgccggctggttgtgtaaaaccctgcggctgaaccagcccggcacacccacgcgcaccgccgtgtga

SEQ ID NO: 53 - DNA, 957 bp, Homo sapiens
atgtccaagccacccgcacccaaccccacacccccccggaacctggactcccggaccttcatcaccattggagacagaaactttgaggtggaggctgatgacttggtgaccatctcagaactgggccgtggagcctatggggtggtagagaaggtgcggcacgcccagagcggcaccatcatggccgtgaagcggatccgggccaccgtgaactcacaggagcagaagcggctgctcatggacctggacatcaacatgcgcacggtcgactgtttctacactgtcaccttctacggggcactattcagagagggagacgtgtggatctgcatggagctcatggacacatccttggacaagttctaccggaaggtgctggataaaaacatgacaattccagaggacatccttggggagattgctgtgtctatcgtgcgggccctggagcatctgcacagcaagctgtcggtgatccacagagatgtgaagccctccaatgtccttatcaacaaggagggccatgtgaagatgtgtgactttggcatcagtggctacttggtggactctgtggccaagacgatggatgccggctgcaagccctacatggcccctgagaggatcaacccagagctgaaccagaagggctacaatgtcaagtccgacgtctggagcctgggcatcaccatgattgagatggccatcctgcggttcccttacgagtcctgggggaccccgttccagcagctgaagcaggtggtggaggagccgtccccccagctcccagccgaccgtttctcccccgagtttgtggacttcactgctcagtgcctgaggaagaaccccgcagagcgtatgagctacctggagctgatggagcaccccttcttcaccttgcacaaaaccaagaagacggacattgctgccttcgtgaaggagatcctgggagaagactcatag

SEQ ID NO: 54 - DNA, 1005 bp, Homo sapiens
atgtctcagtcgaaaggcaagaagcgaaaccctggccttaaaattccaaaagaagcatttgaacaacctcagaccagttccacaccacctcgagatttagactccaaggcttgcatttctattggaaatcagaactttgaggtgaaggcagatgacctggagcctataatggaactgggacgaggtgcgtacggggtggtggagaagatgcggcacgtgcccagcgggcagatcatggcagtgaagcggatccgagccacagtaaatagccaggaacagaaacggctactgatggatttggatatttccatgaggacggtggactgtccattcactgtcaccttttatggcgcactgtttcgggagggtgatgtgtggatctgcatggagctcatggatacatcactagataaattctacaaacaagttattgataaaggccagacaattccagaggacatcttagggaaaatagcagtttctattgtaaaagcattagaacatttacatagtaagctgtctgtcattcacagagacgtcaagccttctaatgtactcatcaatgctctcggtcaagtgaagatgtgcgattttggaatcagtggctacttggtggactctgttgctaaaacaattgatgcaggttgcaaaccatacatggcccctgaaagaataaacccagagctcaaccagaagggatacagtgtgaagtctgacatttggagtctgggcatcacgatgattgagttggccatccttcgatttccctatgattcatggggaactccatttcagcagctcaaacaggtggtagaggagccatcgccacaactcccagcagacaagttctctgcagagtttgttgactttacctcacagtgcttaaagaagaattccaaagaacggcctacatacccagagctaatgcaacatccatttttcaccctacatgaatccaaaggaacagatgtggcatcttttgtaaaactgattcttggagactaa

SEQ ID NO: 55 - DNA, 4539 bp, Homo sapiens
atggcggcggcggcggggaatcgcgcctcgtcgtcgggattcccgggcgccagggctacgagccctgaggcaggcggcggcggaggagccctcaaggcgagcagcgcgcccgcggctgccgcgggactgctgcgggaggcgggcagcgggggccgcgagcgggcggactggcggcggcggcagctgcgcaaagtgcggagtgtggagctggaccagctgcctgagcagccgctcttccttgccgcctcaccgccggcctcctcgacttccccgtcgccggagcccgcggacgcagcggggagtgggaccggcttccagcctgtggcggtgccgccgccccacggagccgcgagccgcggcggcgcccaccttaccgagtcggtggcggcgccggacagcggcgcctcgagtcccgcagcggccgagcccggggagaagcgggcgcccgccgccgagccgtctcctgcagcggcccccgccggtcgtgagatggagaataaagaaactctcaaagggttgcacaagatggatgatcgtccagaggaacgaatgatcagggagaaactgaaggcaacctgtatgccagcctggaagcacgaatggttggaaaggagaaataggcgagggcctgtggtggtaaaaccaatcccagttaaaggagatggatctgaaatgaatcacttagcagctgagtctccaggagaggtccaggcaagtgcggcttcaccagcttccaaaggccgacgcagtccttctcctggcaactccccatcaggtcgcacagtgaaatcagaatctccaggagtaaggagaaaaagagtttccccagtgccttttcagagtggcagaatcacaccaccccgaagagccccttcaccagatggcttctcaccatatagccctgaggaaacaaaccgccgtgttaacaaagtgatgcgggccagactgtacttactgcagcagatagggcctaactctttcctgattggaggagacagcccagacaataaataccgggtgtttattgggcctcagaactgcagctgtgcacgtggaacattctgtattcatctgctatttgtgatgctccgggtgtttcaactagaaccttcagacccaatgttatggagaaaaactttaaagaattttgaggttgagagtttgttccagaaatatcacagtaggcgtagctcaaggatcaaagctccatctcgtaacaccatccagaagtttgtttcacgcatgtcaaattctcatacattgtcatcatctagtacttctacgtctagttcagaaaacagcataaaggatgaagaggaacagatgtgtcctatttgcttgttgggcatgcttgatgaagaaagtcttacagtgtgtgaagacggctgcaggaacaagctgcaccaccactgcatgtcaatttgggcagaagagtgtagaagaaatagagaacctttaatatgtcccctttgtagatctaagtggagatctcatgatttctacagccacgagttgtcaagtcctgtggattccccttcttccctcagagctgcacagcagcaaaccgtacagcagcagcctttggctggatcacgaaggaatcaagagagcaattttaaccttactcattatggaactcagcaaatccctcctgcttacaaagatttagctgagccatggattcaggtgtttggaatggaactcgttggctgcttattttctagaaactggaatgtgagagagatggccctcaggcgtctttcccatgatgtcagtggggccctgctgttggcaaatggggagagcactggaaattctgggggcagcagtggaagcagcccgagtgggggagccaccagtgggtcttcccagaccagtatctcaggagatgtggtggaggcatgctgcagcgttctgtcaatggtctgtgctgaccctgtctacaaagtgtacgttgctgctttaaaaacattgagagccatgctggtatatactccttgccacagtttagcggaaagaatcaaacttcagagacttctccagccagttgtagacaccatcctagtcaaatgtgcagatgccaatagccgcacaagtcagctgtccatatcaacactgttggaactgtgcaaaggccaagcaggagagttggcagttggcagagaaatactaaaagctggatccattggtattggtggtgttgattatgtcttaaattgtattcttggaaaccaaactgaatcaaacaattggcaagaacttcttggccgcctttgtcttatagatagactgttgttggaatttcctgctgaattttatcctcatattgtcagtactgatgtttcacaagctgagcctgttgaaatcaggtataagaagctgctgtccctcttaacctttgctttgcagtccattgataattcccactcaatggttggcaaactttccagaaggatctacttgagttctgcaagaatggttactacagtaccccatgtgttttcaaaactgttagaaatgctgagtgtttccagttccactcacttcaccaggatgcgtcgccgtttgatggctattgcagatgaggtggaaattgccgaagccatccagttgggcgtagaagacactttggatggtcaacaggacagcttcttgcaggcatctgttcccaacaactatctggaaaccacagagaacagttcccctgagtgcacagtccatttagagaaaactggaaaaggattatgtgctacaaaattgagtgccagttcagaggacatttctgagagactggccagcatttcagtaggaccttctagttcaacaacaacaacaacaacaacaacagagcaaccaaagccaatggttcaaacaaaaggcagaccccacagtcagtgtttgaactcctctcctttatctcatcattcccaattaatgtttccagccttgtcaaccccttcttcttctaccccatctgtaccagctggcactgcaacagatgtctctaagcatagacttcagggattcattccctgcagaataccttctgcatctcctcaaacacagcgcaagttttctctacaattccacagaaactgtcctgaaaacaaagactcagataaactttccccagtctttactcagtcaagacccttgccctccagtaacatacacaggccaaagccatctagacctaccccaggtaatacaagtaaacagggagatccctcaaaaaatagcatgacacttgatctgaacagtagttccaaatgtgatgacagctttggctgtagcagcaatagtagtaatgctgttatacccagtgacgagacagtgttcaccccagtagaggagaaatgcagattagatgtcaatacagagctcaactccagtattgaggaccttcttgaagcatctatgccttcaagtgatacaacagtaacttttaagtcagaagttgctgtcctgtctcctgaaaaggctgaaaatgatgatacctacaaagatgatgtgaatcataatcaaaagtgcaaagagaagatggaagctgaagaagaagaagctttagcaattgccatggcaatgtcagcgtctcaggatgccctccccatagttcctcagctgcaggttgaaaatggagaagatatcatcattattcaacaggatacaccagagactctaccaggacataccaaagcaaaacaaccgtatagagaagacactgaatggctgaaaggtcaacagataggccttggagcattttcttcttgttatcaggctcaagatgtgggaactggaactttaatggctgttaaacaggtgacttatgtcagaaacacatcttctgagcaagaagaagtagtagaagcactaagagaagagataagaatgatgagccatctgaatcatccaaacatcattaggatgttgggagccacgtgtgagaagagcaattacaatctcttcattgaatggatggcagggggatcggtggctcatttgctgagtaaatatggagccttcaaagaatcagtagttattaactacactgaacagttactccgtggcctttcgtatctccatgaaaaccaaatcattcacagagatgtcaaaggtgccaatttgctaattgacagcactggtcagagactaagaattgcagattttggagctgcagccaggttggcatcaaaaggaactggtgcaggagagtttcagggacaattactggggacaattgcatttatggcacctgaggtactaagaggtcaacagtatggaaggagctgtgatgtatggagtgttggctgtgctattatagaaatggcttgtgcaaaaccaccatggaatgcagaaaaacactccaatcatcttgctttgatatttaagattgctagtgcaactactgctccatcgatcccttcacatttgtctcctggtttacgagatgtggctcttcgttgtttagaacttcaacctcaggacagacctccatcaagagagctactgaagcatccagtctttcgtactacatggtag

SEQ ID NO: 56 - DNA, 1974 bp, Homo sapiens
atggacgaacaggaggcattgaactcaatcatgaacgatctggtggccctccagatgaaccgacgtcaccggatgcctggatatgagaccatgaagaacaaagacacaggtcactcaaataggcagaaaaaacacaacagcagcagctcagcccttctgaacagccccacagtaacaacaagctcatgtgcaggggccagtgagaaaaagaaatttttgagtgacgtcagaatcaagttcgagcacaacggggagaggcgaattatagcgttcagccggcctgtgaaatatgaagatgtggagcacaaggtgacaacagtatttggacaacctcttgatctacattacatgaacaatgagctctccatcctgctgaaaaaccaagatgatcttgataaagcaattgacattttagatagaagctcaagcatgaaaagccttaggatattgctgttgtcccaggacagaaaccataacagttcctctccccactctggggtgtccagacaggtgcggatcaaggcttcccagtccgcaggggatataaatactatctaccagccccccgagcccagaagcaggcacctctctgtcagctcccagaaccctggccgaagctcacctccccctggctatgttcctgagcggcagcagcacattgcccggcaggggtcctacaccagcatcaacagtgagggggagttcatcccagagaccagcgagcagtgcatgctggatcccctgagcagtgcagaaaattccttgtctggaagctgccaatccttggacaggtcagcagacagcccatccttccggaaatcacgaatgtcccgtgcccagagcttccctgacaacagacaggaatactcagatcgggaaactcagctttatgacaaaggggtcaaaggtggaacctacccccggcgctaccacgtgtctgtgcaccacaaggactacagtgatggcagaagaacatttccccgaatacggcgtcatcaaggcaacttgttcaccctggtgccctccagccgctccctgagcacaaatggcgagaacatgggtctggctgtgcaatacctggacccccgtgggcgcctgcggagtgcggacagcgagaatgccctctctgtgcaggagaggaatgtgccaaccaagtctcccagtgcccccatcaactggcgccggggaaagctcctgggccagggtgccttcggcagggtctatttgtgctatgacgtggacacgggacgtgaacttgcttccaagcaggtccaatttgatccagacagtcctgagacaagcaaggaggtgagtgctctggagtgcgagatccagttgctaaagaacttgcagcatgagcgcatcgtgcagtactatggctgtctgcgggaccgcgctgagaagaccctgaccatcttcatggagtacatgccagggggctcggtgaaagaccagttgaaggcttacggtgctctgacagagagcgtgacccgaaagtacacgcggcagatcctggagggcatgtcctacctgcacagcaacatgattgttcaccgggacattaagggagccaacatcctccgagactctgctgggaatgtaaagctgggggactttggggccagcaaacgcctgcagacgatctgtatgtcggggacgggcatgcgctccgtcactggcacaccctactggatgagccctgaggtgatcagcggcgagggctatggaaggaaagcagacgtgtggagcctgggctgcactgtggtggagatgctgacagagaaaccaccgtgggcagagtatgaagctatggccgccatcttcaagattgccacccagcccaccaatcctcagctgccctcccacatctctgaacatggccgggacttcctgaggcgcatttttgtggaggctcgccagagaccttcagctgaggagctgctcacacaccactttgcacagctcatgtactga

SEQ ID NO: 57 - DNA, 4826 bp, Homo sapiens
atgagagaagccgctgccgcgctggtccctcctcccgcctttgccgtcacgcctgccgccgccatggaggagccgccgccaccgccgccgccgccaccaccgccaccggaacccgagaccgagtcagaacccgagtgctgcttggcggcgaggcaagagggcacattgggagattcagcttgcaagagtcctgaatctgatctagaagacttctccgatgaaacaaatacagagaatctttatggtacctctccccccagcacacctcgacagatgaaacgcatgtcaaccaaacatcagaggaataatgtggggaggccagccagtcggtctaatttgaaagaaaaaatgaatgcaccaaatcagcctccacataaagacactggaaaaacagtggagaatgtggaagaatacagctataagcaggagaaaaagatccgagcagctcttagaacaacagagcgtgatcataaaaaaaatgtacagtgctcattcatgttagactcagtgggtggatctttgccaaaaaaatcaattccagatgtggatctcaataagccttacctcagccttggctgtagcaatgctaagcttccagtatctgtgcccatgcctatagccagacctgcacgccagacttctaggactgactgtccagcagatcgtttaaagttttttgaaactttacgacttttgctaaagcttacctcagtctcaaagaaaaaagacagggagcaaagaggacaagaaaatacgtctggtttctggcttaaccgatctaacgaactgatctggttagagctacaagcctggcatgcaggacggacaattaacgaccaggacttctttttatatacagcccgtcaagccatcccagatattattaatgaaatccttactttcaaagtcgactatgggagcttcgcctttgttagagatagagctggttttaatggtacttcagtagaagggcagtgcaaagccactcctggaacaaagattgtaggttactcaacacatcatgagcatctccaacgccagagggtctcatttgagcaggtaaaacggataatggagctgctagagtacatagaagcactttatccatcattgcaggctcttcagaaggactatgaaaaatatgctgcaaaagacttccaggacagggtgcaggcactctgtttgtggttaaacatcacaaaagacttaaatcagaaattaaggattatgggcactgttttgggcatcaagaatttatcagacattggctggccagtgtttgaaatcccttcccctcgaccatccaaaggtaatgagccggagtatgagggtgatgacacagaaggagaattaaaggagttggaaagtagtacggatgagagtgaagaagaacaaatctctgatcctagggtaccggaaatcagacagcccatagataacagcttcgacatccagtcgcgggactgcatatccaagaagcttgagaggctcgaatctgaggatgattctcttggctggggagcaccagactggagcacagaagcaggctttagtagacattgtctgacttctatttatagaccatttgtagacaaagcactgaagcagatggggttaagaaagttaattttaagacttcacaagctaatggatggttccttgcaaagggcacgtatagcattggtaaagaacgatcgtccagtggagttttctgaatttccagatcccatgtggggttcagattatgtgcagttgtcaaggacaccaccttcatctgaggagaaatgcagtgctgtgtcgtgggaggagctgaaggccatggatttaccttcattcgaacctgccttcctagttctctgccgagtccttctgaatgtcatacatgagtgtctgaagttaagattggagcagagacctgctggagaaccatctctcttgagtattaagcagctggtgagagagtgtaaggaggtcctgaagggcggcctgctgatgaagcagtactaccagttcatgctgcaggaggttctggaggacttggagaagcccgactgcaacattgacgcttttgaagaggatctacataaaatgcttatggtgtattttgattacatgagaagctggatccaaatgctacagcaattacctcaagcatcgcatagtttaaaaaatctgttagaagaagaatggaatttcaccaaagaaataactcattacatacggggaggagaagcacaggccgggaagcttttctgtgacattgcaggaatgctgctgaaatctacaggaagttttttagaatttggcttacaggagagctgtgctgaattttggactagtgcggatgacagcagtgcttccgacgaaatcaggaggtctgttatagagatcagtcgagccctgaaggagctcttccatgaagccagagaaagggcttccaaagcacttggatttgctaaaatgttgagaaaggacctggaaatagcagcagaattcaggctttcagccccagttagagacctcctggatgttctgaaatcaaaacagtatgtcaaggtgcaaattcctgggttagaaaacttgcaaatgtttgttccagacactcttgctgaggagaagagtattattttgcagttactcaatgcagctgcaggaaaggactgttcaaaagattcagatgacgtactcatcgatgcctatctgcttctgaccaagcacggtgatcgagcccgtgattcagaggacagctggggcacctgggaggcacagcctgtcaaagtcgtgcctcaggtggagactgttgacaccctgagaagcatgcaggtggataatcttttactagttgtcatgcagtctgcgcatctcacaattcagagaaaagctttccagcagtccattgagggacttatgactctgtgccaggagcagacatccagtcagccggtcatcgccaaagctttgcagcagctgaagaatgatgcattggagctatgcaacaggataagcaatgccattgaccgcgtggaccacatgttcacatcagaatttgatgctgaggttgatgaatctgaatctgtcaccttgcaacagtactaccgagaagcaatgattcaggggtacaattttggatttgagtatcataaagaagttgttcgtttgatgtctggggagtttagacagaagataggagacaaatatataagctttgcccggaagtggatgaattatgtcctgactaaatgtgagagtggtagaggtacaagacccaggtgggcgactcaaggatttgattttctacaagcaattgaacctgcctttatttcagctttaccagaagatgacttcttgagtttacaagccttgatgaatgaatgcattggccatgtcataggaaaaccacacagtcctgttacaggtttgtaccttgccattcatcggaacagcccccgtcctatgaaggtacctcgatgccatagtgaccctcctaacccacacctcattatccccactccagagggattcagcactcggagcatgccttccgacgcgcggagccatggcagccctgctgctgctgctgctgctgctgctgctgctgttgctgccagtcggcccagcccctctggtggtgactctgtgctgcccaaatccatcagcagtgcccatgataccaggggttccagcgttcctgaaaatgatcgattggcttccatagctgctgaattgcagtttaggtccctgagtcgtcactcaagccccacggaggagcgagatgaaccagcatatccaagaggagattcaagtgggtccacaagaagaagttgggaacttcggacactaatcagccagagtaaagatactgcttctaaactaggacccatagaagctatccagaagtcagtccgattgtttgaagaaaagaggtaccgagaaatgaggagaaagaatatcattggtcaagtttgtgatacgcctaagtcctatgataatgttatgcacgttggcttgaggaaggtgaccttcaaatggcaaagaggaaacaaaattggagaaggccagtatgggaaggtgtacacctgcatcagcgtcgacaccggggagctgatggccatgaaagagattcgatttcaacctaatgaccataagactatcaaggaaactgcagacgaattgaaaatattcgaaggcatcaaacaccccaatctggttcggtattttggtgtggagctccatagagaagaaatgtacatcttcatggagtactgcgatgaggggactttagaagaggtgtcaaggctgggacttcaggaacatgtgattaggctgtattcaaagcagatcaccattgcgatcaacgtcctccatgagcatggcatagtccaccgtgacattaaaggtgccaatatcttccttacctcatctggattaatcaaactgggagattttggatgttcagtaaagctcaaaaacaatgcccagaccatgcctggtgaagtgaacagcaccctggggacagcagcatacatggcacctgaagtcatcactcgtgccaaaggagagggccatgggcgtgcggccgacatctggagtctggggtgtgttgtcatagagatggtgactggcaagaggccttggcatgagtatgagcacaactttcaaattatgtataaagtggggatgggacataagccaccaatccctgaaagattaagccctgaaggaaaggacttcctttctcactgccttgagagtgacccaaagatgagatggaccgccagccagctcctcgaccattcgtttgtcaaggtttgcacagatgaagaatg

SEQ ID NO: 58 - DNA, 3867 bp, Homo sapiens
atggcggggccgtgtccccggtccggggcggagcgcgccggcagctgctggcaggacccgctggccgtggcgctgagccggggccggcagctcgcggcgcccccgggccggggctgcgcgcggagccggccgctcagcgtggtctacgtgctgacccgggagccgcagcccgggctcgagcctcgggagggaaccgaggcggagccgctgcccctgcgctgcctgcgcgaggcttgcgcgcaggtcccccggccgcggccgcccccgcagctgcgcagcctgcccttcgggacgctggagctaggcgacaccgcggctctggatgccttctacaacgcggatgtggtggtgctggaggtgagcagctcgctggtacagccctccctgttctaccaccttggtgtgcgtgagagcttcagcatgaccaacaatgtgctcctctgctcccaggccgacctccctgacctgcaggccctgcgggaggatgttttccagaagaactcggattgcgttggcagctacacactgatcccctatgtggtgacggccactggtcgggtgctgtgtggtgatgcaggccttctgcggggcctggctgatgggctggtacaggctggagtggggaccgaggccctgctcactcccctggtgggccggcttgcccgcctgctggaggccacacccacagactcttgtggctatttccgggagaccattcggcgggacatccggcaggcgcgggagcggttcagtgggccacagctgcggcaggagctggctcgcctgcagcggagactggacagcgtggagctgctgagccccgacatcatcatgaacttgctgctctcctaccgcgatgtgcaggactactcggccatcattgagctggtggagacgctgcaggccttgcccacctgtgatgtggccgagcagcataatgtctgcttccactacacttttgccctcaaccggaggaacaggcctggggaccgggcgaaggccctgtctgtgctgctgccgctggtacagcttgagggctctgtggcgcccgatctgtactgcatgtgtggccgtatctacaaggacatgttcttcagctcgggtttccaggatgctgggcaccgggagcaggcctatcactggtatcgcaaggcttttgacgtagagcccagccttcactcaggcatcaatgcagctgtgctcctcattgctgccgggcagcactttgaggattccaaagagctccggctaataggcatgaagctgggctgcctgctggcccgcaaaggctgcgtggagaagatgcagtattactgggatgtgggtttctacctgggagcccagatcctcgccaatgaccccacccaggtggtgctggctgcagagcagctgtataagctcaatgcccccatatggtacctggtgtccgtgatggagaccttcctgctctaccagcacttcaggcccacgccagagccccctggagggccaccacgccgtgcccacttctggctccacttcttgctacagtcctgccaaccattcaagacagcctgtgcccagggcgaccagtgcttggtgctggtcctggagatgaacaaggtgctgctgcctgcaaagctcgaggttcggggtactgacccagtaagcacagtgaccctgagcctgctggagcctgagacccaggacattccctccagctggaccttcccagtcgcctccatatgcggagtcagcgcctcaaagcgcgacgagcgctgctgcttcctctatgcactccccccggctcaggacgtccagctgtgcttccccagcgtagggcactgccagtggttctgcggcctgatccaggcctgggtgacgaacccggattccacggcgcccgcggaggaggcggagggcgcgggggagatgttggagtttgattatgagtacacggagacgggcgagcggctggtgctgggcaagggcacgtatggggtggtgtacgcgggccgcgatcgccacacgagggtgcgcatcgccatcaaggagatcccggagcgggacagcaggttctctcagcccctgcatgaagagatcgctcttcacagacgcctgcgccacaagaacatagtgcgctatctgggctcagctagccagggcggctaccttaagatcttcatggaggaagtgcctggaggcagcctgtcctccttgctgcggtcggtgtggggacccctgaaggacaacgagagcaccatcagtttctacacccgccagatcctgcagggacttggctacttgcacgacaaccacatcgtgcacagggacataaaaggggacaatgtgctgatcaacaccttcagtgggctgctcaagatttctgacttcggcacctccaagcggctggcaggcatcacaccttgcactgagaccttcacaggaactctgcagtatatggccccagaaatcattgaccagggcccacgcgggtatgggaaagcagctgacatctggtcactgggctgcactgtcattgagatggccacaggtcgcccccccttccacgagctcgggagcccacaggctgccatgtttcaggtgggtatgtacaaggtccatccgccaatgcccagctctctgtcggccgaggcccaagcctttctcctccgaacttttgagccagacccccgcctccgagccagcgcccagacactgctgggggaccccttcctgcagcctgggaaaaggagccgcagccccagctccccacgacatgctccacggccctcagatgccccttctgccagtcccactccttcagccaactcaaccacccagtctcagacattcccgtgccctcaggcaccctctcagcacccacccagccccccgaagcgctgcctcagttatgggggcaccagccagctccgggtgcccgaggagcctgcggccgaggagcctgcgtctccggaggagagttcggggctgagcctgctgcaccaggagagcaagcgtcgggccatgctggccgcagtattggagcaggagctgccagcgctggcggagaatctgcaccaggagcagaagcaagagcagggggcccgtctgggcagaaaccatgtggaagagctgctgcgctgcctcggggcacacatccacactcccaaccgccggcagctcgcccaggagctgcgggcgctgcaaggacggctgagggcccagggccttgggcctgcgcttctgcacagaccgctgtttgccttcccggatgcggtgaagcagatcctccgcaagcgccagatccgtccacactggatgttcgttctggactcactgctcagccgtgctgtgcgggcagccctgggtgtgctaggaccggaggtggagaaggaggcggtctcaccgaggtcagaggagctgagtaatgaaggggactcccagcagagcccaggccagcagagcccgcttccggtggagcccgagcagggccccgctcctctgatggtgcagctgagcctcttgagggcagagactgatcggctgcgcgaaatcctggcggggaaggaacgggagtaccaggccctggtgcagcgggctctacagcggctgaatgaggaagcccggacctatgtcctggccccagagcctccaactgctctttcaacggaccagggcctggtgcagtggctacaggaactgaatgtggattcaggcaccatccaaatgctgttgaaccatagcttcaccctccacactctgctcacctatgccactcgagatgacctcatctacacccgcatcaggggagggatggtatgccgcatctggagggccatcttggcacagcgagcaggatccacaccagtcacctctggaccctga

SEQ ID NO: 59 - DNA, 1740 bp, Homo sapiens
atgtctacagcctctgccgcctcctcctcctcctcgtcttcggccggtgagatgatcgaagccccttcccaggtcctcaactttgaagagatcgactacaaggagatcgaggtggaagaggttgttggaagaggagcctttggagttgtttgcaaagctaagtggagagcaaaagatgttgctattaaacaaatagaaagtgaatctgagaggaaagcgtttattgtagagcttcggcagttatcccgtgtgaaccatcctaatattgtaaagctttatggagcctgcttgaatccagtgtgtcttgtgatggaatatgctgaagggggctctttatataatgtgctgcatggtgctgaaccattgccatattatactgctgcccacgcaatgagttggtgtttacagtgttcccaaggagtggcttatcttcacagcatgcaacccaaagcgctaattcacagggacctgaaaccaccaaacttactgctggttgcaggggggacagttctaaaaatttgtgattttggtacagcctgtgacattcagacacacatgaccaataacaaggggagtgctgcttggatggcacctgaagtttttgaaggtagtaattacagtgaaaaatgtgacgtcttcagctggggtattattctttgggaagtgataacgcgtcggaaaccctttgatgagattggtggcccagctttccgaatcatgtgggctgttcataatggtactcgaccaccactgataaaaaatttacctaagcccattgagagcctgatgactcgttgttggtctaaagatccttcccagcgcccttcaatggaggaaattgtgaaaataatgactcacttgatgcggtactttccaggagcagatgagccattacagtatccttgtcagtattcagatgaaggacagagcaactctgccaccagtacaggctcattcatggacattgcttctacaaatacgagtaacaaaagtgacactaatatggagcaagttcctgccacaaatgatactattaagcgcttagaatcaaaattgttgaaaaatcaggcaaagcaacagagtgaatctggacgtttaagcttgggagcctcccgtgggagcagtgtggagagcttgcccccaacctctgagggcaagaggatgagtgctgacatgtctgaaatagaagctaggatcgccgcaaccacaggcaacggacagccaagacgtagatccatccaagacttgactgtaactggaacagaacctggtcaggtgagcagtaggtcatccagtcccagtgtcagaatgattactacctcaggaccaacctcagaaaagccaactcgaagtcatccatggacccctgatgattccacagataccaatggatcagataactccatcccaatggcttatcttacactggatcaccaactacagcctctagcaccgtgcccaaactccaaagaatctatggcagtgtttgaacagcattgtaaaatggcacaagaatatatgaaagttcaaacagaaattgcattgttattacagagaaagcaagaactagttgcagaactggaccaggatgaaaaggaccagcaaaatacatctcgcctggtacaggaacataaaaagcttttagatgaaaacaaaagcctttctacttactaccagcaatgcaaaaaacaactagaggtcatcagaagtcagcagcagaaacgacaaggcacttcatga

SEQ ID NO: 60 - DNA, 1113 bp, Homo sapiens
atgctgtccaactcccagggccagagcccgccggtgccgttccccgccccggccccgccgccgcagccccccacccctgccctgccgcaccccccggcgcagccgccgccgccgcccccgcagcagttcccgcagttccacgtcaagtccggcctgcagatcaagaagaacgccatcatcgatgactacaaggtcaccagccaggtcctggggctgggcatcaacggcaaagttttgcagatcttcaacaagaggacccaggagaaattcgccctcaaaatgcttcaggactgccccaaggcccgcagggaggtggagctgcactggcgggcctcccagtgcccgcacatcgtacggatcgtggatgtgtacgagaatctgtacgcagggaggaagtgcctgctgattgtcatggaatgtttggacggtggagaactctttagccgaatccaggatcgaggagaccaggcattcacagaaagagaagcatccgaaatcatgaagagcatcggtgaggccatccagtatctgcattcaatcaacattgcccatcgggatgtcaagcctgagaatctcttatacacctccaaaaggcccaacgccatcctgaaactcactgactttggctttgccaaggaaaccaccagccacaactctttgaccactccttgttatacaccgtactatgtggctccagaagtgctgggtccagagaagtatgacaagtcctgtgacatgtggtccctgggtgtcatcatgtacatcctgctgtgtgggtatccccccttctactccaaccacggccttgccatctctccgggcatgaagactcgcatccgaatgggccagtatgaatttcccaacccagaatggtcagaagtatcagaggaagtgaagatgctcattcggaatctgctgaaaacagagcccacccagagaatgaccatcaccgagtttatgaaccacccttggatcatgcaatcaacaaaggtccctcaaaccccactgcacaccagccgggtcctgaaggaggacaaggagcggtgggaggatgtcaaggggtgtcttcatgacaagaacagcgaccaggccacttggctgaccaggttgtga

SEQ ID NO: 61 - DNA, 954 bp, Homo sapiens
atgcgacccgaccgcgctgaggctccaggaccgcccgccatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggttggctagaaggccacggggtgggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctga

SEQ ID NO: 62 - DNA, 2907 bp, Homo sapiens
atggcagaagatgatccatatttgggaaggcctgaacaaatgtttcatttggatccttctttgactcatacaatatttaatccagaagtatttcaaccacagatggcactgccaacagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgctggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacagcggcgtggagacatccttccgcaaactcagctttaccgagtctctgaccagtggtgcctcactgctaactctcaacaaaatgccccatgattatgggcaggaaggacctctagaaggcaaaatttag

SEQ ID NO: 63 - DNA, 2844 bp, Homo sapiens
atggcagtgatggaaatggcctgcccaggtgcccctggctcagcagtggggcagcagaaggaactccccaaagccaaggagaagacgccgccactggggaagaaacagagctccgtctacaagcttgaggccgtggagaagagccctgtgttctgcggaaagtgggagatcctgaatgacgtgattaccaagggcacagccaaggaaggctccgaggcagggccagctgccatctctatcatcgcccaggctgagtgtgagaatagccaagagttcagccccaccttttcagaacgcattttcatcgctgggtccaaacagtacagccagtccgagagtcttgatcagatccccaacaatgtggcccatgctacagagggcaaaatggcccgtgtgtgttggaagggaaagcgtcgcagcaaagcccggaagaaacggaagaagaagagctcaaagtccctggctcatgcaggagtggccttggccaaacccctccccaggacccctgagcaggagagctgcaccatcccagtgcaggaggatgagtctccactcggcgccccatatgttagaaacaccccgcagttcaccaagcctctgaaggaaccaggccttgggcaactctgttttaagcagcttggcgagggcctacggccggctctgcctcgatcagaactccacaaactgatcagccccttgcaatgtctgaaccacgtgtggaaactgcaccacccccaggacggaggccccctgcccctgcccacgcaccccttcccctatagcagactgcctcatcccttcccattccaccctctccagccctggaaacctcaccctctggagtccttcctgggcaaactggcctgtgtagacagccagaaacccttgcctgacccacacctgagcaaactggcctgtgtagacagtccaaagcccctgcctggcccacacctggagcccagctgcctgtctcgtggtgcccatgagaagttttctgtggaggaatacctagtgcatgctctgcaaggcagcgtgagctcaggccaggcccacagcctgaccagcctggccaagacctgggcagcaaggggctccagatcccgggagcccagccccaaaactgaggacaacgagggtgtcctgctcactgagaaactcaagccagtggattatgagtaccgagaagaagtccactgggccacgcaccagctccgcctgggcagaggctccttcggagaggtgcacaggatggaggacaagcagactggcttccagtgcgctgtcaaaaaggtgcggctggaagtatttcgggcagaggagctgatggcatgtgcaggattgacctcacccagaattgtccctttgtatggagctgtgagagaagggccttgggtcaacatcttcatggagctgctggaaggtggctccctgggccagctggtcaaggagcagggctgtctcccagaggaccgggccctgtactacctgggccaggccctggagggtctggaatacctccactcacgaaggattctgcatggggacgtcaaagctgacaacgtgctcctgtccagcgatgggagccacgcagccctctgtgactttggccatgctgtgtgtcttcaacctgatggcctgggaaagtccttgctcacaggggactacatccctggcacagagacccacatggctccggaggtggtgctgggcaggagctgcgacgccaaggtggatgtctggagcagctgctgtatgatgctgcacatgctcaacggctgccacccctggactcagttcttccgagggccgctctgcctcaagattgccagcgagcctccgcctgtgagggagatcccaccctcctgcgcccctctcacagcccaggccatccaagaggggctgaggaaagagcccatccaccgcgtgtctgcagcggagctgggagggaaggtgaaccgggcactacagcaagtgggaggtctgaagagcccttggaggggagaatataaagaaccaagacatccaccgccaaatcaagccaattaccaccagaccctccatgcccagccgagagagctttcgccaagggccccagggccccggccagctgaggagacaacaggcagagcccctaagctccagcctcctctcccaccagagcccccagagccaaacaagtctcctcccttgactttgagcaaggaggagtctgggatgtgggaacccttacctctgtcctccctggagccagcccctgccagaaaccccagctcaccagagcggaaagcaaccgtcccggagcaggaactgcagcagctggaaatagaattattcctcaacagcctgtcccagccattttctctggaggagcaggagcaaattctctcgtgcctcagcatcgacagcctctccctgtcggatgacagtgagaagaacccatcaaaggcctctcaaagctcgcgggacaccctgagctcaggcgtacactcctggagcagccaggccgaggctcgaagctccagctggaacatggtgctggcccgggggcggcccaccgacaccccaagctatttcaatggtgtgaaagtccaaatacagtctcttaatggtgaacacctgcacatccgggagttccaccgggtcaaagtgggagacatcgccactggcatcagcagccagatcccagctgcagccttcagcttggtcaccaaagacgggcagcctgttcgctacgacatggaggtgccagactcgggcatcgacctgcagtgcacactggcccctgatggcagcttcgcctggagctggagggtcaagcatggccagctggagaacaggccctaa

SEQ ID NO: 64 - DNA, 1083 bp, Homo sapiens
atgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattaaccagcttcagcagattatgcgtctgacaggaacaccccccgcttatctcattaacaggatgccaagccatgaggcaagaaactatattcagtctttgactcagatgccgaagatgaactttgcgaatgtatttattggtgccaatcccctggctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtcctga

SEQ ID NO: 65 - DNA, 1656 bp, Homo sapiens
atggctggtgatctttcagcaggtttcttcatggaggaacttaatacataccgtcagaagcagggagtagtacttaaatatcaagaactgcctaattcaggacctccacatgataggaggtttacatttcaagttataatagatggaagagaatttccagaaggtgaaggtagatcaaagaaggaagcaaaaaatgccgcagccaaattagctgttgagatacttaataaggaaaagaaggcagttagtcctttattattgacaacaacgaattcttcagaaggattatccatggggaattacataggccttatcaatagaattgcccagaagaaaagactaactgtaaattatgaacagtgtgcatcgggggtgcatgggccagaaggatttcattataaatgcaaaatgggacagaaagaatatagtattggtacaggttctactaaacaggaagcaaaacaattggccgctaaacttgcatatcttcagatattatcagaagaaacctcagtgaaatctgactacctgtcctctggttcttttgctactacgtgtgagtcccaaagcaactctttagtgaccagcacactcgcttctgaatcatcatctgaaggtgacttctcagcagatacatcagagataaattctaacagtgacagtttaaacagttcttcgttgcttatgaatggtctcagaaataatcaaaggaaggcaaaaagatctttggcacccagatttgaccttcctgacatgaaagaaacaaagtatactgtggacaagaggtttggcatggattttaaagaaatagaattaattggctcaggtggatttggccaagttttcaaagcaaaacacagaattgacggaaagacttacgttattaaacgtgttaaatataataacgagaaggcggagcgtgaagtaaaagcattggcaaaacttgatcatgtaaatattgttcactacaatggctgttgggatggatttgattatgatcctgagaccagtgatgattctcttgagagcagtgattatgatcctgagaacagcaaaaatagttcaaggtcaaagactaagtgccttttcatccaaatggaattctgtgataaagggaccttggaacaatggattgaaaaaagaagaggcgagaaactagacaaagttttggctttggaactctttgaacaaataacaaaaggggtggattatatacattcaaaaaaattaattcatagagatcttaagccaagtaatatattcttagtagatacaaaacaagtaaagattggagactttggacttgtaacatctctgaaaaatgatggaaagcgaacaaggagtaagggaactttgcgatacatgagcccagaacagatttcttcgcaagactatggaaaggaagtggacctctacgctttggggctaattcttgctgaacttcttcatgtatgtgacactgcttttgaaacatcaaagtttttcacagacctacgggatggcatcatctcagatatatttgataaaaaagaaaaaactcttctacagaaattactctcaaagaaacctgaggatcgacctaacacatctgaaatactaaggaccttgactgtgtggaagaaaagcccagagaaaaatgaacgacacacatgttag

SEQ ID NO: 66 - DNA, 1443 bp, Homo sapiens
atgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctga

SEQ ID NO: 67 - DNA, 2514 bp, Homo sapiens
atggctagcaaacgaaaatctacaactccatgcatggttcggacatcacaagtagtagaacaagatgtgcccgaggaagtagacagggccaaagagaaaggaatcggcacaccacagcctgacgtggccaaggacagttgggcagcagaacttgaaaactcttccaaagaaaacgaagtgatagaggtgaaatctatgggggaaagccagtccaaaaaactccaaggtggttatgagtgcaaatactgcccctactccacgcaaaacctgaacgagttcacggagcatgtcgacatgcagcatcccaacgtgattctcaaccccctctacgtgtgtgcagaatgtaacttcacaaccaaaaagtacgactccctatccgaccacaactccaagttccatcccggggaggccaacttcaagctgaagttaattaaacgcaataatcaaactgtcttggaacagtccatcgaaaccaccaaccatgtcgtgtccatcaccaccagtggccctggaactggtgacagtgattctgggatctcggtgagtaaaacccccatcatgaagcctggaaaaccaaaagcggatgccaagaaggtgcccaagaagcccgaggagatcacccccgagaaccacgtggaagggaccgcccgcctggtgacagacacagctgagatcctctcgagactcggcggggtggagctcctccaagacacattaggacacgtcatgccttctgtacagctgccaccaaatatcaaccttgtgcccaaggtccctgtcccactaaatactaccaaatacaactctgccctggatacaaatgccacgatgatcaactctttcaacaagtttccttacccgacccaggctgagttgtcctggctgacagctgcctccaaacacccagaggagcacatcagaatctggtttgccacccagcgcttaaagcatggcatcagctggtccccagaagaggtggaggaggcccggaagaagatgttcaacggcaccatccagtcagtacccccgaccatcactgtgctgcccgcccagttggcccccacaaaggtgacgcagcccatcctccagacggctctaccgtgccagatcctcggccagactagcctggtgctgactcaggtgaccagcgggtcaacaaccgtctcttgctcccccatcacacttgccgtggcaggagtcaccaaccatggccagaagagacccttggtgactccccaagctgcccccgaacccaagcgtccacacatcgctcaggtgccagagcccccacccaaggtggccaaccccccgctcacaccagccagtgaccgcaagaagacaaaggagcagatagcacatctcaaggccagctttctccagagccagttccctgacgatgccgaggtttaccggctcatcgaggtgactggccttgccaggagcgagatcaagaagtggttcagtgaccaccgatatcggtgtcaaaggggcatcgtccacatcaccagcgaatcccttgccaaagaccagttggccatcgcggcctcccgacacggtcgcacgtatcatgcgtacccagactttgccccccagaagttcaaagagaaaacacagggtcaggttaaaatcttggaagacagctttttgaaaagttcttttcctacccaagcagaactggatcggctaagggtggagaccaagctgagcaggagagagatcgactcctggttctcggagaggcggaagcttcgagacagcatggaacaagctgtcttggattccatggggtctggcaaaaaaggccaagatgtgggagcccccaatggtgctctgtctcgactcgaccagctctccggtgcccagttaacaagttctctgcccagcccttcgccagcaattgcaaaaagtcaagaacaggttcatctcctgaggagcacgtttgcaagaacccagtggcctactccccaggagtacgaccagttagcggccaagactggcctggtccgaactgagattgtgcgttggttcaaggagaacagatgcttgctgaaaacgggaaccgtgaagtggatggagcagtaccagcaccagcccatggcagatgatcacggctacgatgccgtagcaaggaaagcaacaaaacccatggccgagagcccaaagaacgggggtgatgtggttccacaatattacaaggaccccaaaaagctctgcgaagaggacttggagaagttggtgaccagggtaaaagtaggcagcgagccagcaaaagactgtttgccagcaaagccctcagaggccacctcagaccggtcagagggcagcagccgggacggccagggtagcgacgagaacgaggagtcgagcgttgtggattacgtggaggtgacggtcggggaggaggatgcgatctcagatagatcagatagctggagtcaggctgcggcagaaggtgtgtcggaactggctgaatcagactccgactgcgtccctgcagaggctggccaggcctag

SEQ ID NO: 68 - DNA, 567 bp, Homo sapiens
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 69 - DNA, 570 bp, Homo sapiens
atgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaa

SEQ ID NO: 70 - DNA, 2016 bp, Homo sapiens
atgcaaccagacatgtccttgaatgtcattaagatgaaatccagtgacttcctggagagtgcagaactggacagcggaggctttgggaaggtgtctctgtgtttccacagaacccagggactcatgatcatgaaaacagtgtacaaggggcccaactgcattgagcacaacgaggccctcttggaggaggcgaagatgatgaacagactgagacacagccgggtggtgaagctcctgggcgtcatcatagaggaagggaagtactccctggtgatggagtacatggagaagggcaacctgatgcacgtgctgaaagccgagatgagtactccgctttctgtaaaaggaaggataattttggaaatcattgaaggaatgtgctacttacatggaaaaggcgtgatacacaaggacctgaagcctgaaaatatccttgttgataatgacttccacattaagatcgcagacctcggccttgcctcctttaagatgtggagcaaactgaataatgaagagcacaatgagctgagggaagtggacggcaccgctaagaagaatggcggcaccctctactacatggcgcccgagcacctgaatgacgtcaacgcaaagcccacagagaagtcggatgtgtacagctttgctgtagtactctgggcgatatttgcaaataaggagccatatgaaaatgctatctgtgagcagcagttgataatgtgcataaaatctgggaacaggccagatgtggatgacatcactgagtactgcccaagagaaattatcagtctcatgaagctctgctgggaagcgaatccggaagctcggccgacatttcctggcattgaagaaaaatttaggcctttttatttaagtcaattagaagaaagtgtagaagaggacgtgaagagtttaaagaaagagtattcaaacgaaaatgcagttgtgaagagaatgcagtctcttcaacttgattgtgtggcagtaccttcaagccggtcaaattcagccacagaacagcctggttcactgcacagttcccagggacttgggatgggtcctgtggaggagtcctggtttgctccttccctggagcacccacaagaagagaatgagcccagcctgcagagtaaactccaagacgaagccaactaccatctttatggcagccgcatggacaggcagacgaaacagcagcccagacagaatgtggcttacaacagagaggaggaaaggagacgcagggtctcccatgacccttttgcacagcaaagaccttacgagaattttcagaatacagagggaaaaggcactgcttattccagtgcagccagtcatggtaatgcagtgcaccagccctcagggctcaccagccaacctcaagtactgtatcagaacaatggattatatagctcacatggctttggaacaagaccactggatccaggaacagcaggtcccagagtttggtacaggccaattccaagtcatatgcctagtctgcataatatcccagtgcctgagaccaactatctaggaaatacacccaccatgccattcagctccttgccaccaacagatgaatctataaaatataccatatacaatagtactggcattcagattggagcctacaattatatggagattggtgggacgagttcatcactactagacagcacaaatacgaacttcaaagaagagccagctgctaagtaccaagctatctttgataataccactagtctgacggataaacacctggacccaatcagggaaaatctgggaaagcactggaaaaactgtgcccgtaaactgggcttcacacagtctcagattgatgaaattgaccatgactatgagcgagatggactgaaagaaaaggtttaccagatgctccaaaagtgggtgatgagggaaggcataaagggagccacggtggggaagctggcccaggcgctccaccagtgttccaggatcgaccttctgagcagcttgatttacgtcagccagaactaa

SEQ ID NO: 71 - DNA, 1569 bp, Homo sapiens
atgagtctgctaaactgtgaaaacagctgtggatccagccagtctgaaagtgactgctgtgtggccatggccagctcctgtagcgctgtaacaaaagatgatagtgtgggtggaactgccagcacggggaacctctccagctcatttatggaggagatccagggatatgatgtagagtttgacccacccctggaaagcaagtatgaatgccccatctgcttgatggcattacgagaagcagtgcaaacgccatgcggccataggttctgcaaagcctgcatcataaaatcaataagggatgcaggtcacaaatgtccagttgacaatgaaatactgctggaaaatcaactatttccagacaattttgcaaaacgtgagattctttctctgatggtgaaatgtccaaatgaaggttgtttgcacaagatggaactgagacatcttgaggatcatcaagcacattgtgagtttgctcttatggattgtccccaatgccagcgtcccttccaaaaattccatattaatattcacattctgaaggattgtccaaggagacaggtttcttgtgacaactgtgctgcatcaatggcatttgaagataaagagatccatgaccagaactgtcctttggcaaatgtcatctgtgaatactgcaatactatactcatcagagaacagatgcctaatcattatgatctagactgccctacagccccaattccatgcacattcagtacttttggttgccatgaaaagatgcagaggaatcacttggcacgccacctacaagagaacacccagtcacacatgagaatgttggcccaggctgttcatagtttgagcgttatacccgactctgggtatatctcagaggtccggaatttccaggaaactattcaccagttagagggtcgccttgtaagacaagaccatcaaatccgggagctgactgctaaaatggaaactcagagtatgtatgtaagtgagctcaaacgaaccattcgaacccttgaggacaaagttgctgaaatcgaagcacagcagtgcaatggaatttatatttggaagattggcaactttggaatgcatttgaaatgtcaagaagaggagaaacctgttgtgattcatagccctggattctacactggcaaacccgggtacaaactgtgcatgcgcttgcaccttcagttaccgactgctcagcgctgtgcaaactatatatccctttttgtccacacaatgcaaggagaatatgacagccacctcccttggcccttccagggtacaatacgccttacaattcttgatcagtctgaagcacctgtaaggcaaaaccacgaagagataatggatgccaaaccagagctgcttgctttccagcgacccacaatcccacggaacccaaaaggttttggctatgtaacttttatgcatctggaagccctaagacaaagaactttcattaaggatgacacattattagtgcgctgtgaggtctccacccgctttgacatgggtagccttcggagggagggttttcagccacgaagtactgatgcaggggtatag

SEQ ID NO: 72 - DNA, 999 bp, Homo sapiens
atggccaaccgttacaccatggatctgactgccatctacgagagcctcctgtcgctgagccctgacgtgcccgtgccatccgaccatggagggactgagtccagcccaggctggggctcctcgggaccctggagcctgagcccctccgactccagcccgtctggggtcacctcccgcctgcctggccgctccaccagcctagtggagggccgcagctgtggctgggtgcccccaccccctggcttcgcaccgctggctccccgcctgggccctgagctgtcaccctcacccacttcgcccactgcaacctccaccaccccctcgcgctacaagactgagctatgtcggaccttctcagagagtgggcgctgccgctacggggccaagtgccagtttgcccatggcctgggcgagctgcgccaggccaatcgccaccccaaatacaagacggaactctgtcacaagttctacctccagggccgctgcccctacggctctcgctgccacttcatccacaaccctagcgaagacctggcggccccgggccaccctcctgtgcttcgccagagcatcagcttctccggcctgccctctggccgccggacctcaccaccaccaccaggcctggccggcccttccctgtcctccagctccttctcgccctccagctccccaccaccacctggggaccttccactgtcaccctctgccttctctgctgcccctggcacccccctggctcgaagagaccccaccccagtctgttgcccctcctgccgaagggccactcctatcagcgtctgggggcccttgggtggcctggttcggaccccctctgtacagtccctgggatccgaccctgatgaatatgccagcagcggcagcagcctggggggctctgactctcccgtcttcgaggcgggagtttttgcaccaccccagcccgtggcagccccccggcgactccccatcttcaatcgcatctctgtttctgagtga

SEQ ID NO: 73 - DNA, 3111 bp, Homo sapiens
atgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgggaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtag

SEQ ID NO: 74 - DNA, 2768 bp, Homo sapiens
atgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgggaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatgggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgtggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtag

SEQ ID NO: 75 - DNA, 3111 bp, Homo sapiens; identical to SEQ ID NO: 73

SEQ ID NO: 76 - DNA, 2940 bp, Homo sapiens
atgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgggaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtag

SEQ ID NO: 77 - DNA, 2940 bp, Homo sapiens
atgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgggaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatgggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtag

SEQ ID NO: 78 - DNA, 3105 bp, Homo sapiens
atggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgggaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtag

SEQ ID NO: 79 - DNA, 588 bp, Homo sapiens
atggggcgcgcgcgcgacgccatcctggatgcgctggagaacctgaccgccgaggagctcaagaagttcaagctgaagctgctgtcggtgccgctgcgcgagggctacgggcgcatcccgcggggcgcgctgctgtccatggacgccttggacctcaccgacaagctggtcagcttctacctggagacctacggcgccgagctcaccgctaacgtgctgcgcgacatgggcctgcaggagatggccgggcagctgcaggcggccacgcaccagggctctggagccgcgccagctgggatccaggcccctcctcagtcggcagccaagccaggcctgcactttatagaccagcaccgggctgcgcttatcgcgagggtcacaaacgttgagtggctgctggatgctctgtacgggaaggtcctgacggatgagcagtaccaggcagtgcgggccgagcccaccaacccaagcaagatgcggaagctcttcagtttcacaccagcctggaactggacctgcaaggacttgctcctccaggccctaagggagtcccagtcctacctggtggaggacctggagcggagctga

SEQ ID NO: 80 - DNA, 531 bp, Homo sapiens
atggggcgcgcgcgcgacgccatcctggatgcgctggagaacctgaccgccgaggagctcaagaagttcaagctgaagctgctgtcggtgccgctgcgcgagggctacgggcgcatcccgcggggcgcgctgctgtccatggacgccttggacctcaccgacaagctggtcagcttctacctggagacctacggcgccgagctcaccgctaacgtgctgcgcgacatgggcctgcaggagatggccgggcagctgcaggcggccacgcaccagggcctgcactttatagaccagcaccgggctgcgcttatcgcgagggtcacaaacgttgagtggctgctggatgctctgtacgggaaggtcctgacggatgagcagtaccaggcagtgcgggccgagcccaccaacccaagcaagatgcggaagctcttcagtttcacaccagcctggaactggacctgcaaggacttgctcctccaggccctaagggagtcccagtcctacctggtggaggacctggagcggagctga

SEQ ID NO: 81 - DNA, 1428 bp, Homo sapiens
atggctgacatgcaaaatctggtagaaagattggagagggcagtgggccgcctggaggcagtatctcatacctctgacatgcaccgtgggtatgcagacagtccttcaaaagcaggagcagctccatatgtgcaggcatttgactcgctgcttgctggtcctgtggcagagtacttgaagatcagtaaagagattgggggagacgtgcagaaacatgcggagatggtccacacaggtttgaagttggagcgagctctgttggttacagcttctcagtgtcaacagccagcagaaaataagctttccgatttgttggcacccatctcagagcagatcaaagaagtgataacctttcgggagaagaaccgaggcagcaagttgtttaatcacctgtcagctgtcagcgaaagtatccaggccctgggctgggtggctatggctcccaagcctggcccttatgtgaaagaaatgaatgatgccgccatgttttatacaaaccgagtcctcaaagagtacaaagatgtggataagaagcatgtagactgggtcaaagcttatttaagtatatggacagagctgcaggcttacattaaggagttccataccaccggactggcctggagcaaaacggggcctgtggcaaaagaactgagcggactgccatctggaccctctgccggatcaggtcctcctccccctccaccaggcccccctcctcccccagtctctaccagttcaggctcagatgagtctgcttcccgctcagcactgttcgcgcagattaatcagggggagagcattacacatgccctgaaacatgtatctgatgacatgaagactcacaagaaccctgccctgaaggctcagagtggtccagtacgcagtggccccaaaccattctctgcacctaaaccccaaaccagcccatcccccaaacgagccacaaagaaggagccagctgtacttgaactggagggcaagaagtggagagtggaaaatcaggaaaatgtttccaacctggtgattgaggacacagagctgaaacaggtggcttacatatacaagtgtgtcaacacgacattgcaaatcaagggcaaaattaactccattacagtagataactgtaagaaacttggcctggtattcgatgacgtggtgggcattgtggagataatcaacagtaaggatgtcaaagttcaggtaatgggtaaagtgccaaccatatccatcaacaaaacagatggctgccatgcttacctgagcaagaattccctggattgtgaaatagtcagtgccaaatcttccgagatgaatgtcctcattcctacagaaggcggtgactttaatgaattcccagttcctgagcagttcaagaccctatggaacgggcagaagttggtcaccacagtgacagaaattgctggataa

SEQ ID NO: 82 - DNA, 1428 bp, Homo sapiens; identical to SEQ ID NO: 81

SEQ ID NO: 83 - DNA, 582 bp, Homo sapiens
atggctgctgaaccagtagaagacaattgcatcaactttgtggcaatgaaatttattgacaatacgctttactttatagctgaagatgatgaaaacctggaatcagattactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactag

SEQ ID NO: 84 - DNA, 570 bp, Homo sapiens
atggctgctgaaccagtagaagacaattgcatcaactttgtggcaatgaaatttattgacaatacgctttactttatagaaaacctggaatcagattactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactag

SEQ ID NO: 85 - DNA, 810 bp, Homo sapiens
atggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaa

SEQ ID NO: 86 - DNA, 597 bp, Homo sapiens
atgcccctaggtctcctgtggctgggcctagccctgttgggggctctgcatgcccaggcccaggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggctga

SEQ ID NO: 87 - DNA, 354 bp, Homo sapiens
atgtctcttgtcagctgtctttcagaagacctgaaggttctgtttttcaggtggggcaagtccgtgggcatcatgttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtag

SEQ ID NO: 88 - DNA, 351 bp, Homo sapiens
atgtctcttgtcagctgtctttcagaagacctggttctgtttttcaggtggggcaagtccgtgggcatcatgttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtag

SEQ ID NO: 89 - DNA, 306 bp, Homo sapiens
atgtggggcaagtccgtgggcatcatgttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtag

SEQ ID NO: 90 - DNA, 282 bp, Homo sapiens
atgttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtag

SEQ ID NO: 91 - DNA, 282 bp, Homo sapiens; identical to SEQ ID NO: 90

SEQ ID NO: 92 - DNA, 345 bp, Homo sapiens
atgacttgcaaaatgtcgcagctggaacgcaacatagagaccatcatcaacaccttccaccaatactctgtgaagctggggcacccagacaccctgaaccagggggaattcaaagagctggtgcgaaaagatctgcaaaattttctcaagaaggagaataagaatgaaaaggtcatagaacacatcatggaggacctggacacaaatgcagacaagcagctgagcttcgaggagttcatcatgctgatggcgaggctaacctgggcctcccacgagaagatgcacgagggtgacgagggccctggccaccaccataagccaggcctcggggagggcaccccctaa